(19) United States
(12) Patent Application Publication
(10) Pub. No.: US 2025/0197372 A1
(43) Pub. Date: Jun. 19, 2025

(54) DEOXYNOJIRIMYCIN DERIVATIVES AS GLUCOSIDASE INHIBITORS

(71) Applicant: EMERGENT PRODUCT DEVELOPMENT GAITHERSBURG INC., Gaithersburg, MD (US)

(72) Inventors: Daniel Cope HILL, Gaithersburg, MD (US); Isaac A. KENNEDY, Gaithersburg, MD (US); Patrick Y.S. LAM, Gaithersburg, MD (US); Anthony M. TRESTON, Gaithersburg, MD (US); Kelly Lyn WARFIELD, Gaithersburg, MD (US)

(73) Assignee: EMERGENT PRODUCT DEVELOPMENT GAITHERSBURG INC., Gaithersburg, MD (US)

(21) Appl. No.: 18/004,262

(22) PCT Filed: Jul. 9, 2021
(86) PCT No.: PCT/US2021/041007

§ 371 Date: Jan. 4, 2023

Related U.S. Application Data
(60) Provisional application No. 62/705,698, filed on Jul. 10, 2020.

(51) Int. Cl. C07D 401/14

(52) U.S. Cl. CPC C07D 401/14

(57) ABSTRACT

The present application provides novel iminosugars and their use as glucosidase inhibitors. The present inventors have discovered that certain deoxynojirimycin derivatives may be effective in inhibiting glucosidases. In particular, such deoxynojirimycin derivatives may be useful for treating a disease or condition where inhibiting glucosidase may be important.

### REFERENCE TO A SEQUENCE LISTING

The instant application contains a Sequence Listing which has been submitted in ASCII format via EFS-Web and is hereby incorporated by reference in its entirety. The Sequence Listing is being concurrently submitted via EFS-Web as an ASCII text file named EPDG_14194SequenceListing.Txt, was created on Feb. 12, 2020, and contains 21 kilobytes.

### FIELD

Embodiments of the present disclosure generally relate to iminosugars and in particular, to N-substituted deoxynojirimycin compounds, and their use as glycosidase inhibitors as well as methods of treating conditions and diseases, for which glycosidase inhibition provides benefit.

### BACKGROUND

Iminosugars, also known as iminosaccharides, includes any analog of a sugar where an oxygen atom in the ring of the structure is replaced with a nitrogen atom. Some iminosugars have been shown to be alpha-glucosidase inhibitors and to have anti-diabetic and anti-viral activity. However, very few iminosugars have made their way to the clinic. Miglitol and miglustat, approved for treating diabetes and Gaucher's disease, respectively, were derived from the glucosidase-inhibiting natural product 1-deoxynojirimycin, are the only examples of FDA approved iminosugar drugs.

A challenge for the development of iminosugars as therapeutics is that the ubiquity of sugars and sugar processing enzymes throughout the body also leads to a variety of side effects. Thus, there is a need to develop iminosugars with selectivity to achieve low levels of or no side-effects in a subject.

### SUMMARY

One aspect of the present disclosure is directed to a compound comprising Formula (I):

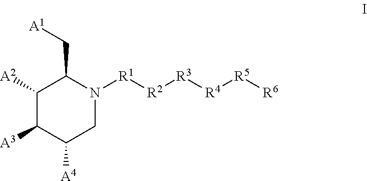

wherein
A¹, A², A³, and A⁴ are independently selected from the group consisting of hydrogen, hydroxyl, and hydroxyl-protecting group;
R¹ is absent or substituted or unsubstituted C₁-C₆ alkyl;
R² is selected from the group consisting of absent, NH, and O;
R³ is selected from the group consisting of substituted or unsubstituted amine, sulfonyl, substituted or unsubstituted C₂-C₆ alkyl, substituted or unsubstituted C₁-C₆ heteroalkyl, substituted or unsubstituted C₁-C₆ alkylene, substituted or unsubstituted C₁-C₆ alkyne, substituted or unsubstituted C₅-C₁₂ aryl, substituted or unsubstituted C₃-C₁₂ cycloalkyl, substituted or unsubstituted C₃-C₁₂ heterocycloalkyl, substituted or unsubstituted C₃-C₁₂ heterocyclyl, substituted or unsubstituted C₅-C₁₂ heteroaryl, substituted or unsubstituted C₃-C₁₂ heterocyclyl, and substituted or unsubstituted C₃-C₁₂ aralkyl;
R⁴ is selected from the group consisting of absent, substituted or unsubstituted amine, substituted or unsubstituted C₁-C₆ alkyl, and substituted or unsubstituted C₁-C₆ heteroalkyl;
R⁵ is selected from the group consisting of absent, NH, NCH₃, and O;
R⁶ is

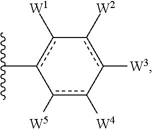

wherein optionally, at least one CW group in

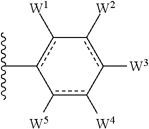

is replaced with N, O, or NH;
wherein
[symbol] are independently a single bond or are absent;
each of W¹, W², W³, W⁴, and W⁵ are independently selected from the group consisting of hydrogen, halogen, hydroxyl, alkoxy, nitro, nitrile, imide, imine, amide, sulfonamide, amino, cyanate, carboxylic acid, substituted or unsubstituted amine, azide, sulfonyl, methoxy, C₁-C₆ alkoxy, substituted or unsubstituted sulfone, substituted or unsubstituted C₁-C₆ alkyl, substituted or unsubstituted C₁-C₆ alkenes, substituted or unsubstituted C₁-C₆ alkynes, substituted or unsubstituted C₁-C₆ heteroalkyl, substituted or unsubstituted C₅-C₁₂ aryl, substituted or unsubstituted C₅-C₁₂ heteroaryl, substituted or unsubstituted C₃-C₁₂ cycloalkyl, substituted or unsubstituted C₃-C₁₂ heterocycloalkyl, substituted or unsubstituted C₃-C₁₂ heterocyclyl substituted or unsubstituted C₃-C₁₂ heterocyclyl, and substituted or unsubstituted C₃-C₁₂ aralkyl;
with the proviso that when R¹, R², R³, and R⁴ taken together are a substituted or unsubstituted C₁-C₆ alkyl linker and R⁵ is NH then W¹, W², W³, W⁴, and W⁵ are not independently selected from the group consisting of hydrogen, nitrile, nitro, amine, substituted or unsubstituted alkanoyl group, adamantyl, substituted or unsubstituted C₃-C₁₂ heterocycloalkyl, and substituted or unsubstituted C₃-C₁₂ heteroalkyl; and
with the proviso that when R¹, R², R³, and R⁴ taken together are a substituted or unsubstituted C₁-C₆ alkyl linker and R⁵ is O then W¹, W², W³, W⁴, and W⁵ are not independently selected from the group consisting of hydrogen and substituted or unsubstituted C₃-C₆ heteroaryl;
or a pharmaceutically acceptable salt.

An additional aspect of the present disclosure is directed to a compound comprising Formula (II):

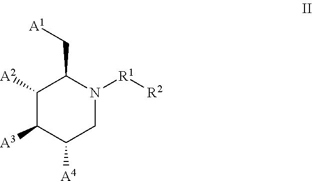

wherein
A¹, A², A³, and A⁴ are independently selected from the group consisting of hydrogen, hydroxyl, and hydroxyl-protecting group;
R¹ is absent or substituted or unsubstituted C₂-C₆ alkyl;
R² is

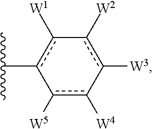

wherein optionally, at least one C or CW group in

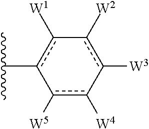

is replaced with N, O, or NH;
or

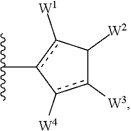

wherein optionally, at least one C or CW group in

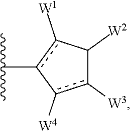

is replaced with N, O, or NH;
wherein
[symbol] are independently a single bond or are absent;
each of W¹, W², W³, W⁴, and W⁵ are independently selected from the group consisting of hydrogen, ketone, substituted or unsubstituted C₁-C₆ alkyl, and W¹ and W², W² and W³, W³ and W⁴, or W⁴ and W⁵ taken together form a substituted or unsubstituted C₅-C₆ aromatic ring;
or a pharmaceutically acceptable salt thereof.

Other aspects and iterations of the present disclosure are described in more detail below.

### BRIEF DESCRIPTION OF THE DRAWINGS

The accompanying drawings, which are incorporated herein and form a part of the specification, illustrate the present disclosure and, together with the description, further serve to explain the principles of the disclosure and to enable a person skilled in the pertinent art to make and use the disclosure.

FIG. 1 shows the inhibition (% neutralization of activity) by compound 1012 for Glu I and Glu II.

FIG. 2 shows the inhibition (% neutralization of activity) by compound 1317 for GAA and GBA.

FIG. 3 shows the inhibition (% neutralization of activity) by compound 1003 for maltase and sucrase.

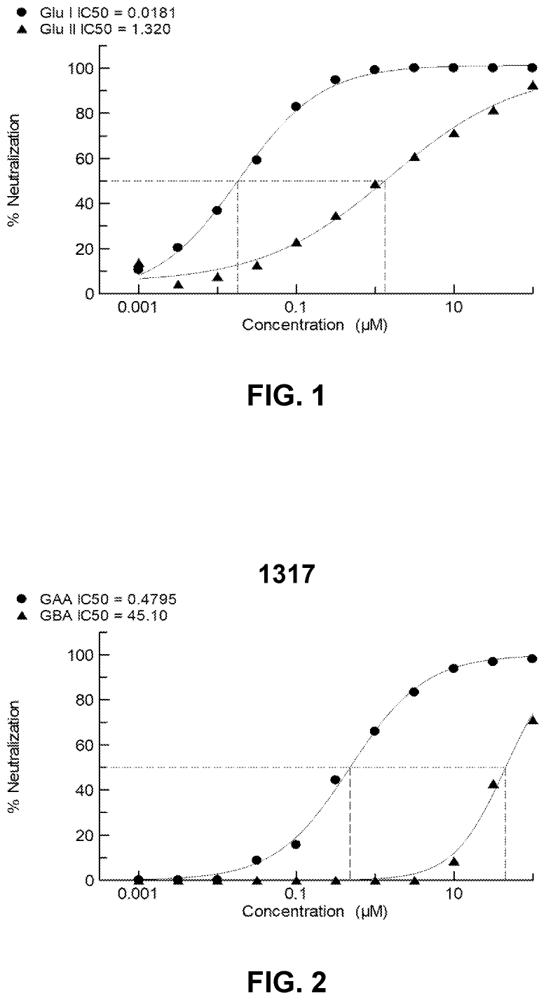

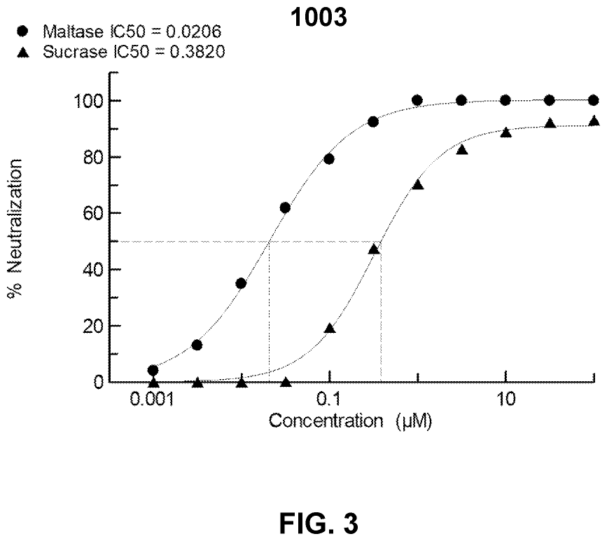

### DETAILED DESCRIPTION

### I. Definitions

In order that the present disclosure can be more readily understood, certain terms are defined. As used in this application, except as otherwise expressly provided herein, each of the following terms shall have the meaning set forth below. Additional definitions are set forth throughout the application.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, “a” or “an” means one or more unless otherwise specified.

As used herein, “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds. John Wiley & Sons, New York: 2001, the entire contents of which are hereby incorporated by reference.

In general, “substituted” refers to an alkyl, alkenyl, alkynyl, aryl, or ether group, as defined below (e.g., an alkyl group) in which one or more bonds to a hydrogen atom contained therein are replaced by a bond to non-hydrogen or non-carbon atoms. In some aspects, one or more carbon atoms in an alkyl, alkenyl, alkynyl, or aryl group is replaced with a non-carbon atom. Examples of non-carbon atoms include, but are not limited to, O, N, S, F, Cl, Br, and I. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some aspects, a substituted group is substituted with 1, 2, 3, 4, 5, or 6 substituents. Examples of substituent groups include, but are not limited to: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like. Substituted groups also include where a carbon atom in an alkyl, alkenyl, alkynyl, or aryl group is replaced with one or more nitrogen, sulfur, or oxygen atoms.

As used herein, “alkyl” groups include straight chain and branched alkyl groups having from 1 to about 20 carbon atoms, and typically from 1 to 12 carbons or, in some aspects, from 1 to 8 carbon atoms. As employed herein, alkyl groups include cycloalkyl groups as defined herein. Alkyl groups are substituted or unsubstituted. Examples of straight chain alkyl groups include, but are not limited to, methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups are substituted one or more times with, for example, amino, thio, hydroxy, cyano, alkoxy, and/or halo groups such as F, Cl, Br, and I. As used herein the term haloalkyl is an alkyl group having one or more halo groups. In some aspects, haloalkyl refers to a per-haloalkyl group. As used herein, cycloalkyl groups are cyclic alkyl groups such as, but not limited to, cyclopropyl, cyclobutyl, cyclopentyl, cyclohexyl, cycloheptyl, and cyclooctyl groups. In some aspects, the cycloalkyl group has 3 to 8 ring members, whereas in other aspects the number of ring carbon atoms range from 3 to 5, 6, or 7. Cycloalkyl groups are substituted or unsubstituted. Cycloalkyl groups further include polycyclic cycloalkyl groups such as, but not limited to, norbornyl, adamantyl, bornyl, camphenyl, isocamphenyl, and carenyl groups, and fused rings such as, but are not limited to, decalinyl, and the like. Cycloalkyl groups also include rings that are substituted with straight or branched chain alkyl groups as defined above. Representative substituted cycloalkyl groups are mono-substituted or substituted more than once, such as, but not limited to: 2,2-; 2,3-; 2,4-; 2,5-; or 2,6-disubstituted cyclohexyl groups or mono-, di-, or tri-substituted norbornyl or cycloheptyl groups, which is substituted with, for example, alkyl, alkoxy, amino, thio, hydroxy, cyano, and/or halo groups.

As used herein, “alkenyl” groups are straight chain, branched or cyclic alkyl groups having 2 to about 20 carbon atoms, and further including at least one double bond. In some aspects alkenyl groups have from 1 to 12 carbons, or, typically, from 1 to 8 carbon atoms. Alkenyl groups are substituted or unsubstituted. Alkenyl groups include, but are not limited to, vinyl, propenyl, 2-butenyl, 3-butenyl, isobutenyl, cyclohexenyl, cyclopentenyl, cyclohexadienyl, butadienyl, pentadienyl, and hexadienyl groups among others. Alkenyl groups are substituted similarly to alkyl groups. Divalent alkenyl groups, i.e., alkenyl groups with two points of attachment, include, but are not limited to, CH CH═CH₂, C═CH₂, or C═CHCH₃.

As used herein, “alkyne” groups are straight chain or branched alkyl groups having 2 to about 20 carbon atoms, and further including at least one triple bond. In some aspects, alkyne groups have from 1 to 12 carbons, or, typically, from 1 to 8 carbon atoms. Alkyne groups are substituted similarly to alkyl groups.

As used herein, “aryl” or “aromatic,” groups are cyclic aromatic hydrocarbons that do not contain heteroatoms. Aryl groups include, but are not limited to, monocyclic, bicyclic, and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some aspects, aryl groups contain 6 to 14 carbons, and in others from 6 to 12 or even 6 to 10 carbon atoms in the ring portions of the groups. The phrase “aryl groups” includes groups containing fused rings, such as fused aromatic-aliphatic ring systems (e.g., indanyl, tetrahydronaphthyl, and the like). Aryl groups is substituted or unsubstituted.

As used herein, “heteroalkyl” group include straight and branched chain alkyl groups as defined herein and further include 1, 2, 3, 4, 5, or 6 heteroatoms independently selected from oxygen, sulfur, and nitrogen. Thus, heteroalkyl groups include 1 to 12 carbon atoms, 1 to 10 carbons or, in some aspects, from 1 to 8, or 1, 2, 3, 4, 5, or 6 carbon atoms, or any range therein (e.g., 1-4). Example of heteroalkyl groups include, but are not limited to, (CH₂CH₂O)₁₋₅CH₃, (CH₂)₁₋₆O(CH₂)₁₋₆ CH₃, (CH₂)₁₋₆NRₐ(CH₂)₁₋₆ CH₃, (CH₂)₁₋₆S(CH₂)₁₋₆ CH₃, (CH₂)₁₋₆O(CH₂)₁₋₆O(CH₂)₁₋₆ CH₃, (CH₂)₁₋₆ NRₐ(CH₂)₁₋₆NRₐ(CH₂)₁₋₆CH₃, (CH₂)₁₋₆O(CH₂)₁₋₆O(CH₂)₁₋₆O(CH₂)₁₋₆CH₃, (CH₂)₁₋₆NRₐ(CH₂)₁₋₆NRₐ(CH₂)₁₋₆NRₐ(CH₂)₁₋₆CH₃, with the total number of carbon atoms in the heteroalkyl group being 1 to 12 and R^{a} is a hydrogen or a substituted or unsubstituted alkyl, alkenyl, aryl or aralkyl group. Other examples of heteroalkyl groups include, but are not limited to, groups having different heteroatoms in a single group. Such examples of heteroalkyl groups include, but are not limited to, (CH₂)₁₋₆S(CH₂)₁₋₆O(CH₂)₁₋₆, (CH₂)₁₋₆NRₐ(CH₂)₁₋₆)O(CH₂)₁₋₆, (CH₂)₁₋₆O(CH₂)₁₋₆NRₐ(CH₂)₁₋₆S(CH₂)₁₋₆, (CH₂)₁₋₆NRₐ(CH₂)₁₋₆O(CH₂)₁₋₆S(CH₂)₁₋₆, with the total number of carbon atoms in the heteroalkyl group being 1 to 12. In some aspects, heteroalkyl groups include, but are not limited to, polyoxyethylene groups, such as (OCH₂CH₂)₁₋₅CH₃, for example, O(CH₂)₂O(CH₂)₂OCH₃, O(CH₂)₂O(CH₂)₂O(CH₂)₂OCH₃, O(CH₂)₂O(CH₂)₂O(CH₂)₂O(CH₂)₂OCH₃.

As used herein, “hydroxyl-protecting” group signifies any group commonly used for the temporary protection of hydroxyl functions, such as for example, alkoxycarbonyl, acyl, alkylsilyl, alkylarylsilyl, and alkoxyalkyl groups, and a protected hydroxyl group is a hydroxyl function derivatized by such a protecting group. Alkoxycarbonyl protecting groups are groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term ‘acyl’ signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro, or alkyl substituted benzoyl group.

As used herein, “aralkyl” groups are substituted aryl groups in which an alkyl group as defined herein has a hydrogen or carbon bond of the alkyl group replaced with a bond to an aryl group as defined above. In some aspects, aralkyl groups contain 7 to 14 carbon atoms, 7 to 10 carbon atoms, e.g., 7, 8, 9, or 10 carbon atoms or any range therein (e.g., 7-8). Aralkyl groups are substituted or unsubstituted. Substituted aralkyl groups are substituted at the alkyl, the aryl or both the alkyl and aryl portions of the group. Representative substituted and unsubstituted alkaryl groups include but are not limited to alkylphenyl such as methylphenyl, (chloromethyl)phenyl, chloro(chloromethyl)phenyl, or fused alkaryl groups such as 5-ethylnaphthalenyl.

As used herein, “heterocyclyl” groups are non-aromatic ring compounds containing 3 or more ring members, of which one or more is a heteroatom such as, but not limited to, N, O, and S. In some aspects, the heterocyclyl group contains 1, 2, 3 or 4 heteroatoms. In some aspects, heterocyclyl groups include mono-, bi-, and tricyclic rings having 3 to 16 ring members, whereas other such groups have 3 to 6, 3 to 10, 3 to 12, or 3 to 14 ring members. Heterocyclyl groups encompass partially unsaturated and saturated ring systems, such as, for example, imidazolinyl and imidazolidinyl groups. The phrase also includes bridged polycyclic ring systems containing a heteroatom such as, but not limited to, quinuclidyl. The phrase also includes heterocyclyl groups that have other groups, such as alkyl, oxo or halo groups, bonded to one of the ring members, referred to as “substituted heterocyclyl groups.” Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, pyrrolinyl, piperidyl, piperazinyl, morpholinyl, thiomorpholinyl, tetrahydropyranyl, and tetrahydrothiopyranyl groups. Representative substituted heterocyclyl groups are mono-substituted or substituted more than once, such as, but not limited to, morpholinyl groups, which are 2-, 3-, 4-, 5-, or 6-substituted, or disubstituted with various substituents such as those listed above. The heteroatom(s) may also be in oxidized form, if chemically possible.

As used herein, “heteroaryl” groups are aromatic ring compounds containing 5 or more ring members, of which, one or more is a heteroatom such as, but are not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, imidazolyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. The phrase “heteroaryl groups” includes fused ring compounds and also includes heteroaryl groups that have other groups bonded to one of the ring members, such as alkyl groups, referred to as “substituted heteroaryl groups.” Representative substituted heteroaryl groups are substituted one or more times with various substituents such as those listed above. The heteroatom(s) may also be in oxidized form, if chemically possible.

As used herein, the terms “halogen” or “halo” as used herein refers to bromine, chlorine, fluorine, or iodine. The term “halide” as used herein refers to the anion of a halogen, such as bromide, chloride, fluoride, and iodide.

As used herein, the terms “alkoxy” refers to a substituted or unsubstituted alkyl group bonded to an oxygen atom. Examples include, but are not limited to, methoxy and ethoxy. Representative substituted alkoxy groups are substituted one or more times with substituents such as those listed above, such as methoxymethyl and fluoromethoxy.

The aforementioned description of the specific aspects fully reveal the general nature of the disclosure that others can, by applying knowledge within the skill of the art, readily modify and/or adapt for various applications, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the described exemplary aspects.

The contents of all references, patents and published patent applications cited throughout this application are expressly incorporated herein by reference.

### II. Compounds

The present inventors have discovered that certain iminosugars are potent inhibitors of the endoplasmic reticulum (ER) alpha-glucosidase enzymes.

a. Compounds Comprising Formula (I)

Provided herein are compounds comprising Formula (I):

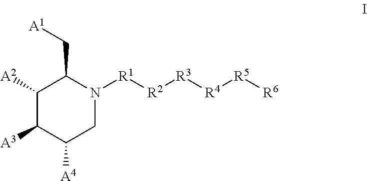

wherein
A¹, A², A³, and A⁴ are independently selected from the group consisting of hydrogen, hydroxyl, and hydroxyl-protecting group;
R¹ is absent or substituted or unsubstituted C₁-C₆ alkyl;
R² is selected from the group consisting of absent, NH, and oxygen;
R³ is selected from the group consisting of substituted or unsubstituted amine, sulfonyl, substituted or unsubstituted C₂-C₆ alkyl, substituted or unsubstituted C₁-C₆ heteroalkyl, substituted or unsubstituted C₁-C₆ alkylene, substituted or unsubstituted C₁-C₆ alkyne, substituted or unsubstituted C₅-C₁₂ aryl, substituted or unsubstituted C₃-C₁₂ cycloalkyl, substituted or unsubstituted C₃-C₁₂ heterocycloalkyl, substituted or unsubstituted C₃-C₁₂ heterocyclyl, substituted or unsubstituted C₅-C₁₂ heteroaryl, substituted or unsubstituted C₃-C₁₂ heterocyclyl, and substituted or unsubstituted C₃-C₁₂ aralkyl;
R⁴ is selected from the group consisting of absent, substituted or unsubstituted amine, substituted or unsubstituted C₁-C₆ alkyl, and substituted or unsubstituted C₁-C₆ heteroalkyl;
R⁵ is selected from the group consisting of absent, NH, NCH₃, and oxygen;
R⁶ is

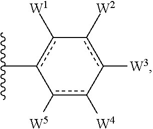

wherein optionally, at least one CW group in

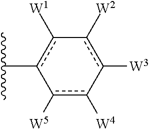

is replaced with N, O, or NH;
   wherein [symbol]
   are independently a single bond or are absent;
   each of W¹, W², W³, W⁴, and W⁵ are independently selected from the group consisting of hydrogen, halogen, hydroxyl, alkoxy, nitro, nitrile, imide, imine, amide, sulfonamide, amino, cyanate, carboxylic acid, substituted or unsubstituted amine, azide, sulfonyl, methoxy, C₁-C₆ alkoxy, substituted or unsubstituted sulfone, substituted or unsubstituted C₁-C₆ alkyl, substituted or unsubstituted C₁-C₆ alkenes, substituted or unsubstituted C₁-C₆ alkynes, substituted or unsubstituted C₁-C₆ heteroalkyl, substituted or unsubstituted C₅-C₁₂ aryl, substituted or unsubstituted C₅-C₁₂ heteroaryl, substituted or unsubstituted C₃-C₁₂ cycloalkyl, substituted or unsubstituted C₃-C₁₂ heterocycloalkyl, substituted or unsubstituted C₃-C₁₂ heterocyclyl substituted or unsubstituted C₃-C₁₂ heterocyclyl, and substituted or unsubstituted C₃-C₁₂ aralkyl;
   with the proviso that when R¹, R², R³, and R⁴ taken together are a substituted or unsubstituted C₁-C₆ alkyl linker and R⁵ is NH then W¹, W², W³, W⁴, and W⁵ are not independently selected from the group consisting of hydrogen, nitrile, nitro, amine, substituted or unsubstituted alkanoyl group, adamantyl, substituted or unsubstituted C₃-C₁₂ heterocycloalkyl, and substituted or unsubstituted C₃-C₁₂ heteroalkyl; and with the proviso that when R¹, R², R³, and R⁴ taken together are a substituted or unsubstituted C₁-C₆ alkyl linker and R⁵ is O then W¹, W², W³, W⁴, and W⁵ are not independently selected from the group consisting of hydrogen and substituted or unsubstituted C₃-C₆ heteroaryl;
   or a pharmaceutically acceptable salt.

In one aspect, a compound comprising Formula (I) comprises any of the preceding compounds of Formula (I), wherein A¹, A², A³, and A⁴ are independently selected from the group consisting of hydroxyl and hydroxyl-protecting group.

In a further aspect, a compound comprising Formula (I) comprises any of the preceding compounds of Formula (I), wherein R¹ is absent or an unsubstituted C₁-C₄ alkyl.

In an additional aspect, a compound comprising Formula (I) comprises any of the preceding compounds of Formula (I), wherein R¹ is selected from the group consisting of absent, methyl, ethyl, propyl, and butyl.

In another aspect, a compound comprising Formula (I) comprises any of the preceding compounds of Formula (I), wherein R² is absent or NH.

In another aspect, a compound comprising Formula (I) comprises any of the preceding compounds of Formula (I), wherein R³ is selected from the group consisting of substituted or unsubstituted C₆-C₁₂ aryl, substituted or unsubstituted C₁-C₆ alkyne, substituted or unsubstituted C₂-C₆ alkyl, substituted or unsubstituted C₁-C₆ heteroalkyl, substituted or unsubstituted C₃-C₁₂ cycloalkyl, and substituted or unsubstituted C₃-C₁₂ heterocycloalkyl.

In a further aspect, a compound comprising Formula (I) comprises any of the preceding compounds of Formula (I), wherein R³ is selected from the group consisting of substituted or unsubstituted amine, sulfonyl, substituted or unsubstituted C₆-C₁₂ aryl, unsubstituted C₂-C₄ alkyl, unsubstituted C₁-C₄ alkyne, unsubstituted C₁-C₆ heteroalkyl, substituted C₁-C₆ heteroalkyl, substituted or unsubstituted C₅-C₁₂ heteroaryl, substituted or unsubstituted C₅-C₁₂ heterocyclyl, and substituted or unsubstituted C₅-C₁₀ heteroaryl.

In an additional aspect, a compound comprising Formula (I) comprises any of the preceding compounds of Formula (I), wherein R³ is selected from the group consisting of oxygen, methyl, ethyl, propyl, butyl, phenyl, biphenyl, naphthalene, indole, chlorophenyl, methoxyphenyl, tert-butylphenyl, cyclohexylphenyl, diphenylmethane, isopropylbenzene, azetidine, thiophene, pyridine, pyrazine, pyrimidine, piperazine, imidazole, pyrazole, furan, thiazole, cyclohexylphenyl, 2,3-dihydro-1H-indene, 2,3-dihydrobenzo[b]1,4-dioxine, bicyclo[1.1.1]pentan-1-yl)methyl, benzothiazole, 1,4-dioxane, 1,2,3-triazole, 1,2,3,4-tetrahydronaphthalene, N(CH₂)₅OCH₃, N(CH₂)₂CONH₃, CHC(O)NH₂, C(O)C(O), NHC(O), SO₂, C(OH)C(OH), CH₂SO₂, NHSO₂NH, NHC(O)NH, OCH₂, and CH₂O.

In a further aspect, a compound comprising Formula (I) comprises any of the preceding compounds of Formula (I), wherein R⁴ is selected from the group consisting of absent, unsubstituted amine, unsubstituted C₁-C₄ alkyl, and unsubstituted C₁-C₄ heteroalkyl.

In an additional aspect, a compound comprising Formula (I) comprises any of the preceding compounds of Formula (I), wherein R⁴ is selected from the group consisting of absent, methyl, ethyl, propyl, butyl, NH(CH₂)₂, and O(CH₂)₂.

In another aspect, a compound comprising Formula (I) comprises any of the preceding compounds of Formula (I), wherein R⁵ is selected from the group consisting of absent, NH, and NCH₃.

In another aspect, a compound comprising Formula (I) comprises any of the preceding compounds of Formula (I), wherein R⁶ is

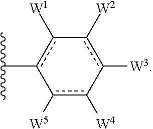

In another aspect, a compound comprising Formula (I) comprises any of the preceding compounds of Formula (I), wherein [symbol] are single bonds.

In another aspect, a compound comprising Formula (I) comprises any of the preceding compounds of Formula (I), wherein W¹, W², W³, W⁴, and W⁵ are independently selected from the group consisting of hydrogen, halogen, nitro, nitrile, carboxylic acid, cyanate, substituted amine, substituted or unsubstituted C₃-C₁₂ cycloalkyl, substituted or unsubstituted C₁-C₆ alkyl, substituted or unsubstituted C₁-C₆ heteroalkyl, substituted or unsubstituted sulfone, substituted or unsubstituted C₅-C₁₂ heteroaryl, substituted or unsubstituted C₅-C₁₂ heterocyclyl, and substituted or unsubstituted C₅-C₁₀ heteroaryl.

In a further aspect, a compound comprising Formula (I) comprises any of the preceding compounds of Formula (I), wherein W¹, W², W³, W⁴, and W⁵ are independently selected from the group consisting of hydrogen, methyl, ethyl, propyl, nitro, aldehyde, bromo, chloro, fluoro, cyano, nitrile, methoxy, difluoromethyl, trifluoromethyl, tert-butyl, tert-butoxy, cyclopropyl, cyclohexyl, methanesulfonyl, methyltetrahydrofuran, hydromethyl, acetamide, N,N-dimethylacetamide, tert-butoxy, cyclohexene, cyclohexane, phenyl, tetrahydrofuran, pyridazine, morpholine, pyrrole, pyrimidine, furan, azide, methoxyethyl, oxazole, oxadiazole, imidazole, isoxazole, cyclopropanesulfonamide, sulfonylacetamide, (dimethylamino)methyl, methylcyano, anisole, phenyl, benzyl, N-chlorosuccinimide, 1,1-dimethoxyethane, 2,6-dimethylmorpholine, 2-oxa-5-azabicyclo[2.2.1]heptan-5-yl, 2-oxa-6-azaspiro[3.4]octan-6-yl, (1R,5S)-3-oxa-8-azabicyclo[3.2.1]octan-8-yl, 2-oxa-7-azaspiro[3.5]nonan-7-yl, methyl(2-nitro-4-[1,2-oxazolidin-2-yl])methyl, 2,3-dihydro-1,4-dioxine, (1,2-oxazolidin-2-yl)methyl, 2,3-dihydrofuran, 1,2,3-oxadiazole, 5-propyl-1,2,4-oxadiazole, 5-propyl,1,2,4-(1,2-oxazolidin-2-yl)methyl, 3,6-dihydro-2H-pyran, 5-phenyl-1,2,4-oxadiazole, 5-cyclobutyl-1,2,4-oxadiazole, 5-butyl-1,2,4-oxadiazole, 1,2,3-triazole, 2-oxa-7-azaspiro[3.5]nonane, 2-oxa-6-azaspiro[3.4]octane, COOH, COOCH₃, CONHAc, C(NH)NH₂, NHCOCH₃, SO₂NS(CH₃)₂, C(O)NH₂, SO₂NHCOCH₃, SO₂NH₂, CONHCa, C(O)OCH₃, C(NH)C(O)H, CCCH₃, CH₂C(O)O(CH₂)₂CH₃, (CH₃)₂OH, NHCN, SCN, CH₂CCH, and NHSO₂CH₃.

In another aspect, a compound comprising Formula (I) comprises any of the preceding compounds of Formula (I), wherein A¹, A², A³, and A⁴ are hydroxyl; R¹ is CH₂; R² is absent or CH; R³ is phenyl, biphenyl, or (CH₂)₄; R⁴ is CH₂; R⁵ is NH; R⁶ is

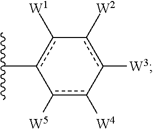

[symbol]

are single bonds; and each of W¹, W², W³, W⁴, and W⁵ are independently selected from the group consisting of hydrogen, bromo, chloro, nitro, cyano, methyl, azide, methoxy, cyclohexene, pyrrole, furan, phenyl, pyridine, pyrimidine, pyridazine, oxazole, 1,4-dioxene, thiophene, CH₂-isoxazole, 3-oxa-8-azabicyclo[3.2.1]octanyl, 4-(5,6-dihydro-2H-thiopyran), 1,2,4-oxadiazole, 4-(pyrimidin-2-yl)morpholine, 3-(5-propyl-1,2,4-oxadiazole), 3-(5-cyclobutane-1,2,4-oxadiazole), 4-(piperidin-1-yl)pyrimidine, 4-(piperidin-1-yl)morpholine, 3,6-dihydro-2H-pyran, 2,3-dihydro-1,4-dioxine, 3-cyclopropylpyridazine, 5-propyl-1,2,4-oxadiazole, 5-cyclobutyl-1,2,4-oxadiazole, 1,2,4-oxadiazole, 3,6-dihydro-2H-thiopyran, 4-(pyrimidin-2-yl)morpholine, and 5-benzyl-1,2,4-oxadizaole.

In an additional aspect, a compound comprising Formula (I), wherein A¹, A², A³, and A⁴ are independently selected from the group consisting of hydroxyl, and hydroxyl-protecting group; R¹ is absent or a substituted or unsubstituted C₁-C₆ alkyl; R² is absent or NH; R³ is selected from the group consisting of substituted or unsubstituted C₆-C₁₂ aryl, substituted or unsubstituted C₂-C₆ alkyl, substituted or unsubstituted C₁-C₆ heteroalkyl, substituted or unsubstituted C₃-C₁₂ cycloalkyl, and substituted or unsubstituted C₃-C₁₂ heterocycloalkyl; R⁴ is selected from the group consisting of absent, substituted or unsubstituted amine, substituted or unsubstituted C₁-C₆ alkyl, and substituted or unsubstituted C₁-C₆ heteroalkyl; R⁵ is selected from the group consisting of absent, NH, and NCH₃; R⁶ is

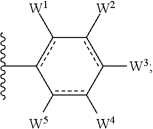

[symbol]

is single bond; and W¹, W², W³, W⁴, and W⁵ is independently selected from the group consisting of hydrogen, halogen, nitro, nitrile, carboxylic acid, cyanate, substituted amine, substituted or unsubstituted C₃-C₁₂ cycloalkyl, substituted or unsubstituted C₁-C₆ alkyl, substituted or unsubstituted C₁-C₆ heteroalkyl, substituted or unsubstituted sulfone, substituted or unsubstituted C₅-C₁₂ heteroaryl, substituted or unsubstituted C₅-C₁₂ heterocyclyl, and substituted or unsubstituted C₅-C₁₀ heteroaryl; with the proviso that when R¹, R², R³, and R⁴ taken together are a substituted or unsubstituted C₁-C₆ alkyl linker and R⁵ is NH then W¹, W², W³, W⁴, and W⁵ are not independently selected from the group consisting of hydrogen, nitrile, nitro, amine, substituted or unsubstituted alkanoyl group, adamantyl, substituted or unsubstituted C₃-C₁₂ heterocycloalkyl, and substituted or unsubstituted C₃-C₁₂ heteroalkyl; and with the proviso that when R¹, R², R³, and R⁴ taken together are a substituted or unsubstituted C₁-C₆ alkyl linker and R⁵ is O then W¹, W², W³, W⁴, and W⁵ are not independently selected from the group consisting of hydrogen and substituted or unsubstituted C₃-C₆ heteroaryl; or a pharmaceutically acceptable salt.

In an additional aspect, a compound comprising Formula (I), wherein A¹, A², A³, and A⁴ are independently selected from the group consisting of hydroxyl, and hydroxyl-protecting group; R¹ is absent or an unsubstituted C₁-C₄ alkyl; R² is absent or NH; R³ is selected from the group consisting of substituted or unsubstituted amine, sulfonyl, substituted or unsubstituted C₆-C₁₂ aryl, unsubstituted C₂-C₄ alkyl, unsubstituted C₁-C₆ heteroalkyl, substituted C₁-C₆ heteroalkyl, substituted or unsubstituted C₅-C₁₂ heteroaryl, substituted or unsubstituted C₅-C₁₂ heterocyclyl, and substituted or unsubstituted C₅-C₁₀ heteroaryl; R⁴ is selected from the group consisting of absent, unsubstituted amine, unsubstituted C₁-C₄ alkyl, and unsubstituted C₁-C₄ heteroalkyl; R⁵ is selected from the group consisting of absent, NH, and NCH₃; R⁶ is

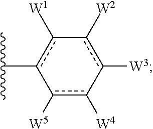

[symbol]

is single bond; and W¹, W², W³, W⁴, and W⁵ is independently selected from the group consisting of hydrogen, halogen, nitro, nitrile, carboxylic acid, cyanate, substituted amine, substituted or unsubstituted C₃-C₁₂ cycloalkyl, substituted or unsubstituted C₁-C₆ alkyl, substituted or unsubstituted C₁-C₆ heteroalkyl, substituted or unsubstituted sulfone, substituted or unsubstituted C₅-C₁₂ heteroaryl, substituted or unsubstituted C₅-C₁₂ heterocyclyl, and substituted or unsubstituted C₅-C₁₀ heteroaryl; with the proviso that when R¹, R², R³, and R⁴ taken together are a substituted or unsubstituted C₁-C₆ alkyl linker and R⁵ is NH then W¹, W², W³, W⁴, and W⁵ are not independently selected from the group consisting of hydrogen, nitrile, nitro, amine, substituted or unsubstituted alkanoyl group, adamantyl, substituted or unsubstituted C₃-C₁₂ heterocycloalkyl, and substituted or unsubstituted C₃-C₁₂ heteroalkyl; and with the proviso that when R¹, R², R³, and R⁴ taken together are a substituted or unsubstituted C₁-C₆ alkyl linker and R⁵ is O then W¹, W², W³, W⁴, and W⁵ are not independently selected from the group consisting of hydrogen and substituted or unsubstituted C₃-C₆ heteroaryl; or a pharmaceutically acceptable salt.

In an additional aspect, a compound comprising Formula (I), wherein A¹, A², A³, and A⁴ are independently selected from the group consisting of hydroxyl, and hydroxyl-protecting group; R¹ is selected from the group consisting of absent, methyl, ethyl, propyl, and butyl; R² is absent or NH; R³ is selected from the group consisting of oxygen, ethyl, propyl, butyl, phenyl, biphenyl, naphthalene, indole, chlorophenyl, methoxyphenyl, tert-butylphenyl, cyclohexylphenyl, diphenylmethane, isopropylbenzene, azetidine, thiophene, pyridine, pyrazine, pyrimidine, piperazine, imidazole, pyrazole, furan, thiazole, cyclohexylphenyl, 2,3-dihydro-1H-indene, 2,3-dihydrobenzo[b]1,4-dioxine, bicyclo[1.1.1]pentan-1-yl)methyl, benzothiazole, 1,4-dioxane, 1,2,3-triazole, 1,2,3,4-tetrahydronaphthalene, N(CH₂)₅OCH₃, N(CH₂)₂CONH₃, CHC(O)NH₂, C(O)C(O), NHC(O), SO₂, C(OH)C(OH), CH₂SO₂, NHSO₂NH, NHC(O)NH, OCH₂, and CH₂O; R⁴ is selected from the group consisting of absent, methyl, ethyl, propyl, butyl, NH(CH₂)₂, and O(CH₂)₂; R⁵ is selected from the group consisting of absent, NH, and NCH₃; R⁶ is

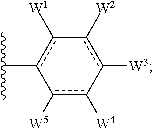

[symbol]

is single bond; and W¹, W², W³, W⁴, and W⁵ is independently selected from the group consisting of hydrogen, methyl, ethyl, propyl, nitro, aldehyde, bromo, chloro, fluoro, cyano, nitrile, methoxy, difluoromethyl, trifluoromethyl, tert-butyl, tert-butoxy, cyclopropyl, cyclohexyl, methanesulfonyl, methyltetrahydrofuran, hydromethyl, acetamide, N,N-dimethylacetamide, tert-butoxy, cyclohexene, cyclohexane, phenyl, tetrahydrofuran, pyridazine, morpholine, pyrrole, pyrimidine, furan, azide, methoxyethyl, oxazole, oxadiazole, imidazole, isoxazole, cyclopropanesulfonamide, sulfonylacetamide, (dimethylamino)methyl, methylcyano, anisole, phenyl, benzyl, N-chlorosuccinimide, 1,1-dimethoxyethane, 2,6-dimethylmorpholine, 2-oxa-5-azabicyclo[2.2.1]heptan-5-yl, 2-oxa-6-azaspiro[3.4]octan-6-yl, (1R,5S)-3-oxa-8-azabicyclo[3.2.1]octan-8-yl, 2-oxa-7-azaspiro[3.5]nonan-7-yl, methyl(2-nitro-4-[1,2-oxazolidin-2-yl])methyl, 2,3-dihydro-1,4-dioxine, (1,2-oxazolidin-2-yl)methyl, 2,3-dihydrofuran, 1,2,3-oxadiazole, 5-propyl-1,2,4-oxadiazole, 5-propyl,1,2,4-(1,2-oxazolidin-2-yl)methyl, 3,6-dihydro-2H-pyran, 5-phenyl-1,2,4-oxadiazole, 5-cyclobutyl-1,2,4-oxadiazole, 5-butyl-1,2,4-oxadiazole, 1,2,3-triazole, 2-oxa-7-azaspiro[3.5]nonane, 2-oxa-6-azaspiro[3.4]octane, COOH, COOCH₃, CONHAc, C(NH)NH₂, NHCOCH₃, SO₂NS(CH₃)₂, C(O)NH₂, SO₂NHCOCH₃, SO₂NH₂, CONHCa, C(O)OCH₃, C(NH)C(O)H, CCCH₃, CH₂C(O)O(CH₂)₂CH₃, (CH₃)₂OH, NHCN, SCN, CH₂CCH, and NHSO₂CH₃; with the proviso that when R¹, R², R³, and R⁴ taken together are a substituted or unsubstituted C₁-C₆ alkyl linker and R⁵ is NH then W¹, W², W³, W⁴, and W⁵ are not independently selected from the group consisting of hydrogen, nitrile, nitro, amine, substituted or unsubstituted alkanoyl group, adamantyl, substituted or unsubstituted C₃-C₁₂ heterocycloalkyl, and substituted or unsubstituted C₃-C₁₂ heteroalkyl; and with the proviso that when R¹, R², R³, and R⁴ taken together are a substituted or unsubstituted C₁-C₆ alkyl linker and R⁵ is O then W¹, W², W³, W⁴, and W⁵ are not independently selected from the group consisting of hydrogen and substituted or unsubstituted C₃-C₆ heteroaryl; or a pharmaceutically acceptable salt.

In an aspect, a compound comprising Formula (I) is selected from the compounds listed in Table 1.

| # | Structure |
|---|---|
| 1001 | 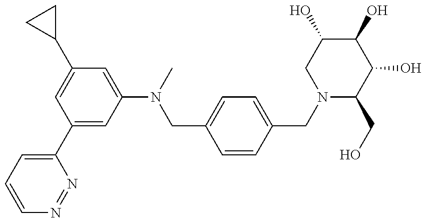 [SMILES: CN(Cc1ccc(CN2C[C@H](O)[C@@H](O)[C@H](O)[C@H]2CO)cc1)c1cc(-c2cccnn2)cc(C2CC2)c1] |
| 1002 | 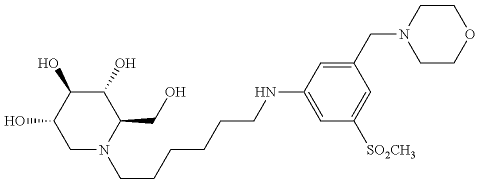 [SMILES: CS(=O)(=O)c1cc(CN2CCOCC2)cc(NCCCCCCN2C[C@H](O)[C@@H](O)[C@H](O)[C@H]2CO)c1] |
| 1003 | 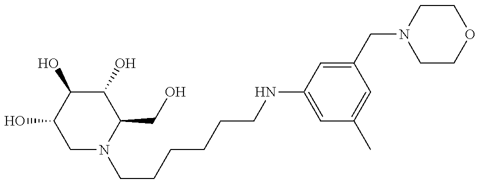 [SMILES: Cc1cc(CN2CCOCC2)cc(NCCCCCCN2C[C@H](O)[C@@H](O)[C@H](O)[C@H]2CO)c1] |
| 1004 | 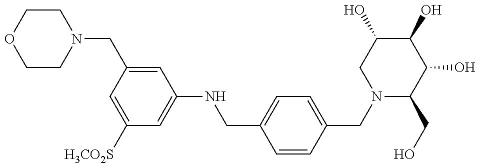 |
| 1005 | 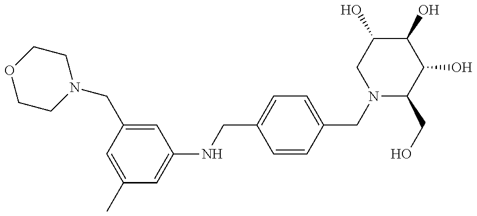 [SMILES: Cc1cc(CN2CCOCC2)cc(NCc2ccc(CN3C[C@H](O)[C@@H](O)[C@H](O)[C@H]3CO)cc2)c1] |
| 1006 | 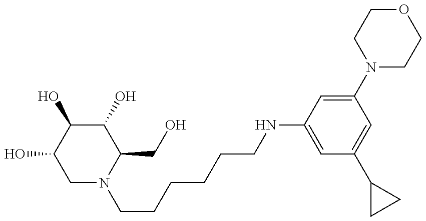 [SMILES: OC[C@@H]1[C@@H](O)[C@H](O)[C@@H](O)CN1CCCCCCNc1cc(C2CC2)cc(N2CCOCC2)c1] |
| 1007 | 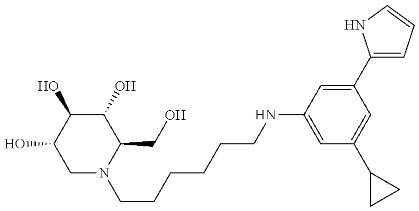 [SMILES: OC[C@@H]1[C@@H](O)[C@H](O)[C@@H](O)CN1CCCCCCNc1cc(-c2ccc[nH]2)cc(C2CC2)c1] |
| 1008 | 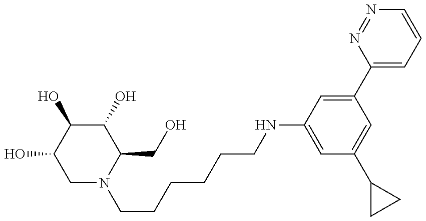 [SMILES: OC[C@@H]1[C@@H](O)[C@H](O)[C@@H](O)CN1CCCCCCNc1cc(-c2cccnn2)cc(C2CC2)c1] |
| 1009 | 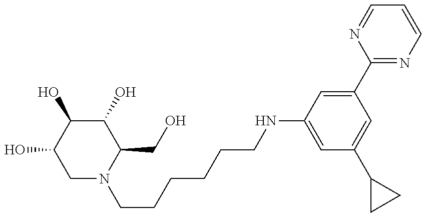 [SMILES: OC[C@@H]1[C@@H](O)[C@H](O)[C@@H](O)CN1CCCCCCNc1cc(-c2ncccn2)cc(C2CC2)c1] |
| 1010 | 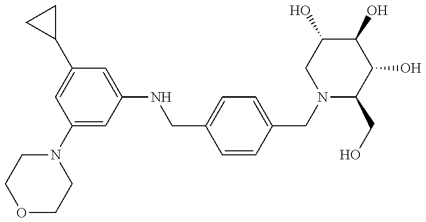 [SMILES: OC[C@@H]1[C@@H](O)[C@H](O)[C@@H](O)CN1Cc1ccc(CNc2cc(C3CC3)cc(N3CCOCC3)c2)cc1] |
| 1011 | 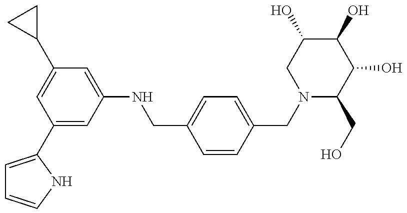 [SMILES: OC[C@@H]1[C@@H](O)[C@H](O)[C@@H](O)CN1Cc1ccc(CNc2cc(-c3ccc[nH]3)cc(C3CC3)c2)cc1] |
| 1012 | 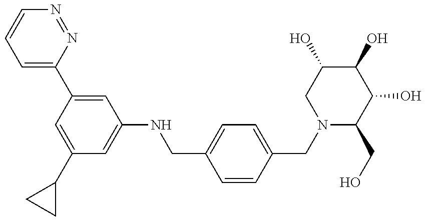 [SMILES: OC[C@@H]1[C@@H](O)[C@H](O)[C@@H](O)CN1Cc1ccc(CNc2cc(-c3cccnn3)cc(C3CC3)c2)cc1] |
| 1013 | 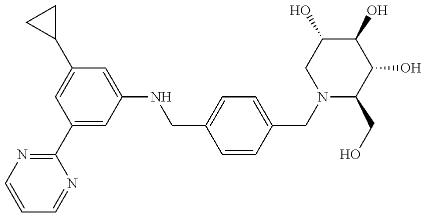 [SMILES: OC[C@@H]1[C@@H](O)[C@H](O)[C@@H](O)CN1Cc1ccc(CNc2cc(-c3ncccn3)cc(C3CC3)c2)cc1] |
| 1014 | 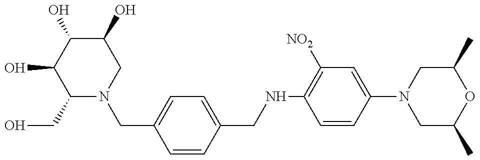 [SMILES: C[C@@H]1CN(c2ccc(NCc3ccc(CN4C[C@H](O)[C@@H](O)[C@H](O)[C@H]4CO)cc3)c([N+](=O)[O-])c2)C[C@H](C)O1] |
| 1015 | 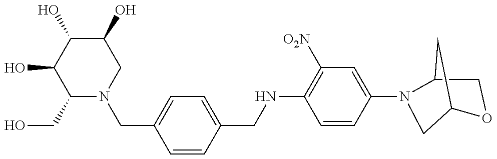 [SMILES: O=[N+]([O-])c1cc(N2CC3CC2CO3)ccc1NCc1ccc(CN2C[C@H](O)[C@@H](O)[C@H](O)[C@H]2CO)cc1] |
| 1016 | 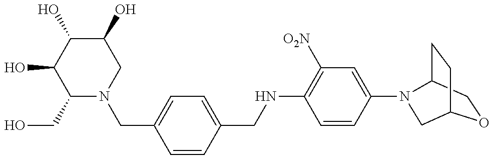 [SMILES: O=[N+]([O-])c1cc(N2CC3CCC2CO3)ccc1NCc1ccc(CN2C[C@H](O)[C@@H](O)[C@H](O)[C@H]2CO)cc1] |
| 1017 | 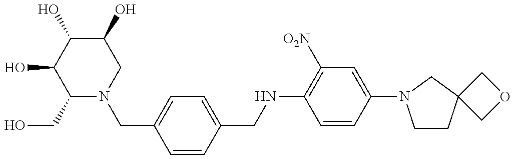 [SMILES: O=[N+]([O-])c1cc(N2CCC3(COC3)C2)ccc1NCc1ccc(CN2C[C@H](O)[C@@H](O)[C@H](O)[C@H]2CO)cc1] |
| 1018 | 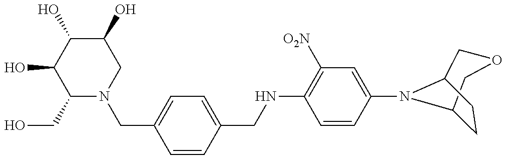 [SMILES: O=[N+]([O-])c1cc(N2C3CCC2COC3)ccc1NCc1ccc(CN2C[C@H](O)[C@@H](O)[C@H](O)[C@H]2CO)cc1] |
| 1019 | 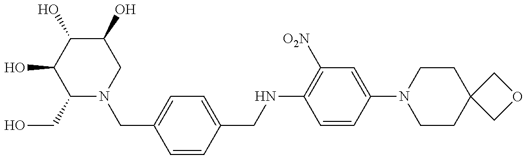 [SMILES: O=[N+]([O-])c1cc(N2CCC3(CC2)COC3)ccc1NCc1ccc(CN2C[C@H](O)[C@@H](O)[C@H](O)[C@H]2CO)cc1] |
| 1020 | 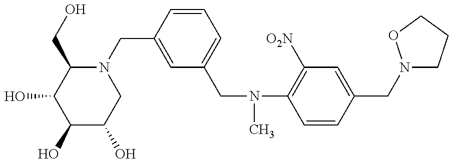 [SMILES: CN(Cc1cccc(CN2C[C@H](O)[C@@H](O)[C@H](O)[C@H]2CO)c1)c1ccc(CN2CCCO2)cc1[N+](=O)[O-]] |
| 1021 | 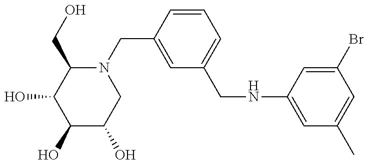 [SMILES: Cc1cc(Br)cc(NCc2cccc(CN3C[C@H](O)[C@@H](O)[C@H](O)[C@H]3CO)c2)c1] |
| 1022 | 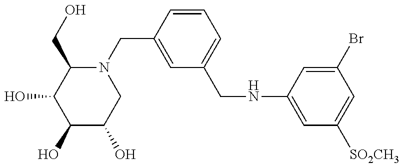 [SMILES: CS(=O)(=O)c1cc(Br)cc(NCc2cccc(CN3C[C@H](O)[C@@H](O)[C@H](O)[C@H]3CO)c2)c1] |
| 1023 | 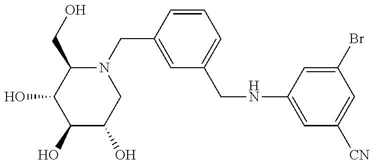 [SMILES: N#Cc1cc(Br)cc(NCc2cccc(CN3C[C@H](O)[C@@H](O)[C@H](O)[C@H]3CO)c2)c1] |
| 1024 | 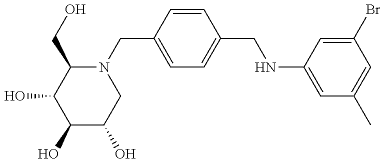 [SMILES: Cc1cc(Br)cc(NCc2ccc(CN3C[C@H](O)[C@@H](O)[C@H](O)[C@H]3CO)cc2)c1] |
| 1025 | 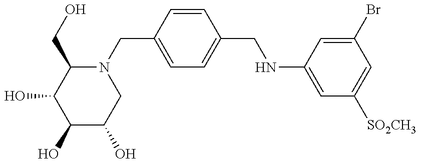 [SMILES: CS(=O)(=O)c1cc(Br)cc(NCc2ccc(CN3C[C@H](O)[C@@H](O)[C@H](O)[C@H]3CO)cc2)c1] |
| 1026 | 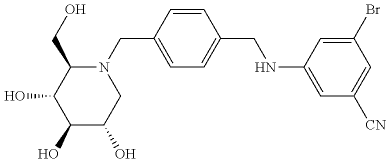 [SMILES: N#Cc1cc(Br)cc(NCc2ccc(CN3C[C@H](O)[C@@H](O)[C@H](O)[C@H]3CO)cc2)c1] |
| 1027 | 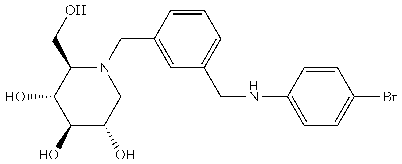 [SMILES: OC[C@@H]1[C@@H](O)[C@H](O)[C@@H](O)CN1Cc1cccc(CNc2ccc(Br)cc2)c1] |
| 1028 | 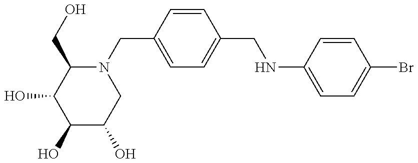 [SMILES: OC[C@@H]1[C@@H](O)[C@H](O)[C@@H](O)CN1Cc1ccc(CNc2ccc(Br)cc2)cc1] |
| 1029 | 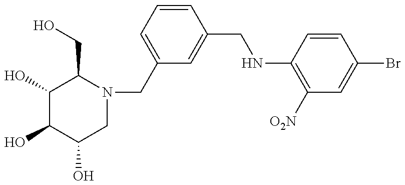 [SMILES: O=[N+]([O-])c1cc(Br)ccc1NCc1cccc(CN2C[C@H](O)[C@@H](O)[C@H](O)[C@H]2CO)c1] |
| 1030 | 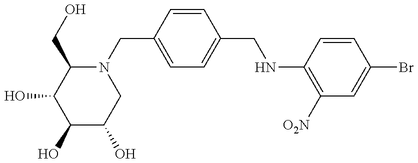 [SMILES: O=[N+]([O-])c1cc(Br)ccc1NCc1ccc(CN2C[C@H](O)[C@@H](O)[C@H](O)[C@H]2CO)cc1] |
| 1031 | 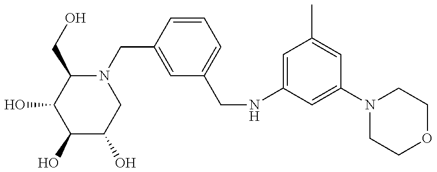 [SMILES: Cc1cc(NCc2cccc(CN3C[C@H](O)[C@@H](O)[C@H](O)[C@H]3CO)c2)cc(N2CCOCC2)c1] |
| 1032 | 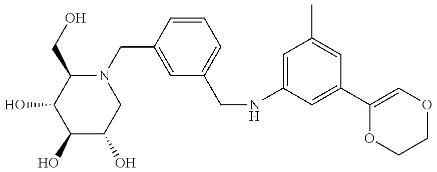 [SMILES: Cc1cc(NCc2cccc(CN3C[C@H](O)[C@@H](O)[C@H](O)[C@H]3CO)c2)cc(C2=COCCO2)c1] |
| 1033 | 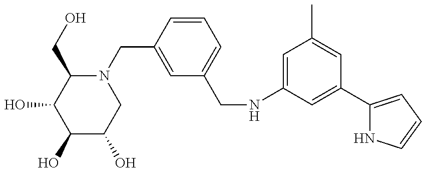 [SMILES: Cc1cc(NCc2cccc(CN3C[C@H](O)[C@@H](O)[C@H](O)[C@H]3CO)c2)cc(-c2ccc[nH]2)c1] |
| 1034 | 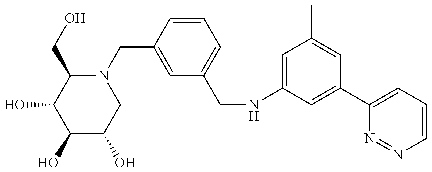 [SMILES: Cc1cc(NCc2cccc(CN3C[C@H](O)[C@@H](O)[C@H](O)[C@H]3CO)c2)cc(-c2cccnn2)c1] |
| 1035 | 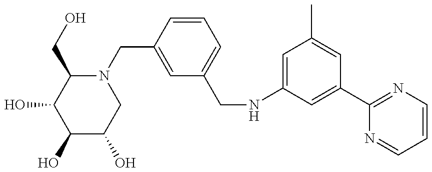 [SMILES: Cc1cc(NCc2cccc(CN3C[C@H](O)[C@@H](O)[C@H](O)[C@H]3CO)c2)cc(-c2ncccn2)c1] |
| 1036 | 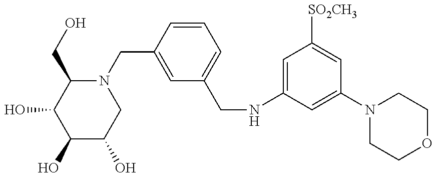 [SMILES: CS(=O)(=O)c1cc(NCc2cccc(CN3C[C@H](O)[C@@H](O)[C@H](O)[C@H]3CO)c2)cc(N2CCOCC2)c1] |
| 1037 | 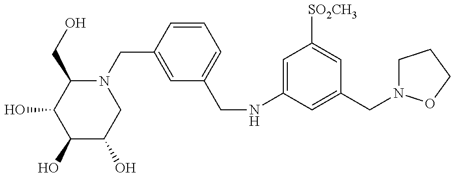 [SMILES: CS(=O)(=O)c1cc(CN2CCCO2)cc(NCc2cccc(CN3C[C@H](O)[C@@H](O)[C@H](O)[C@H]3CO)c2)c1] |
| 1038 | 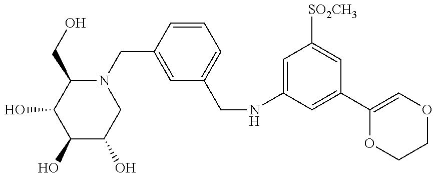 [SMILES: CS(=O)(=O)c1cc(NCc2cccc(CN3C[C@H](O)[C@@H](O)[C@H](O)[C@H]3CO)c2)cc(C2=COCCO2)c1] |
| 1039 | 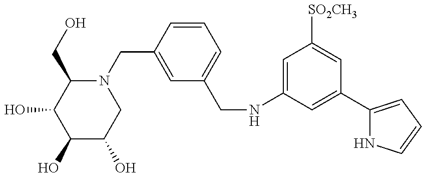 [SMILES: CS(=O)(=O)c1cc(NCc2cccc(CN3C[C@H](O)[C@@H](O)[C@H](O)[C@H]3CO)c2)cc(-c2ccc[nH]2)c1] |
| 1040 | 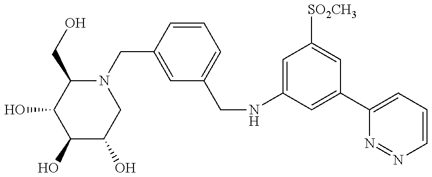 [SMILES: CS(=O)(=O)c1cc(NCc2cccc(CN3C[C@H](O)[C@@H](O)[C@H](O)[C@H]3CO)c2)cc(-c2cccnn2)c1] |
| 1041 | 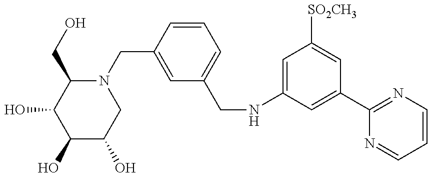 [SMILES: CS(=O)(=O)c1cc(NCc2cccc(CN3C[C@H](O)[C@@H](O)[C@H](O)[C@H]3CO)c2)cc(-c2ncccn2)c1] |
| 1042 | 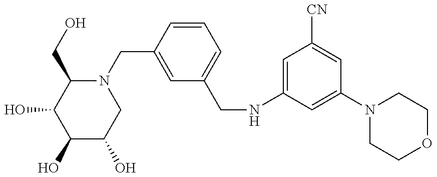 [SMILES: N#Cc1cc(NCc2cccc(CN3C[C@H](O)[C@@H](O)[C@H](O)[C@H]3CO)c2)cc(N2CCOCC2)c1] |
| 1043 | 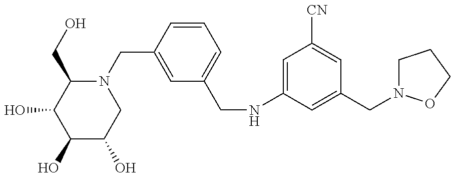 [SMILES: N#Cc1cc(CN2CCCO2)cc(NCc2cccc(CN3C[C@H](O)[C@@H](O)[C@H](O)[C@H]3CO)c2)c1] |
| 1044 | 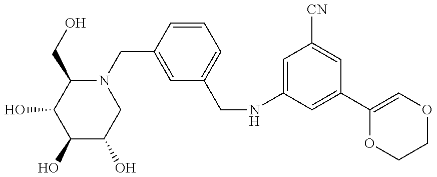 [SMILES: N#Cc1cc(NCc2cccc(CN3C[C@H](O)[C@@H](O)[C@H](O)[C@H]3CO)c2)cc(C2=COCCO2)c1] |
| 1045 | 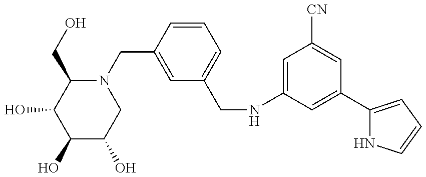 [SMILES: N#Cc1cc(NCc2cccc(CN3C[C@H](O)[C@@H](O)[C@H](O)[C@H]3CO)c2)cc(-c2ccc[nH]2)c1] |
| 1046 | 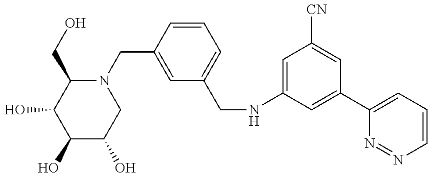 [SMILES: N#Cc1cc(NCc2cccc(CN3C[C@H](O)[C@@H](O)[C@H](O)[C@H]3CO)c2)cc(-c2cccnn2)c1] |
| 1047 | 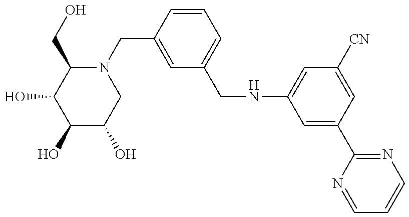 [SMILES: N#Cc1cc(NCc2cccc(CN3C[C@H](O)[C@@H](O)[C@H](O)[C@H]3CO)c2)cc(-c2ncccn2)c1] |
| 1048 | 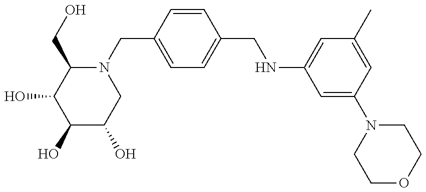 [SMILES: Cc1cc(NCc2ccc(CN3C[C@H](O)[C@@H](O)[C@H](O)[C@H]3CO)cc2)cc(N2CCOCC2)c1] |
| 1049 | 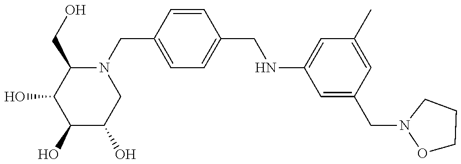 [SMILES: Cc1cc(CN2CCCO2)cc(NCc2ccc(CN3C[C@H](O)[C@@H](O)[C@H](O)[C@H]3CO)cc2)c1] |
| 1050 | 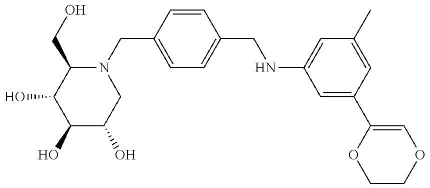 [SMILES: Cc1cc(NCc2ccc(CN3C[C@H](O)[C@@H](O)[C@H](O)[C@H]3CO)cc2)cc(C2=COCCO2)c1] |
| 1051 | 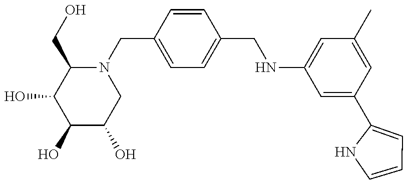 [SMILES: Cc1cc(NCc2ccc(CN3C[C@H](O)[C@@H](O)[C@H](O)[C@H]3CO)cc2)cc(-c2ccc[nH]2)c1] |
| 1052 | 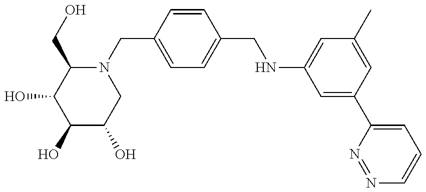 [SMILES: Cc1cc(NCc2ccc(CN3C[C@H](O)[C@@H](O)[C@H](O)[C@H]3CO)cc2)cc(-c2cccnn2)c1] |
| 1053 | 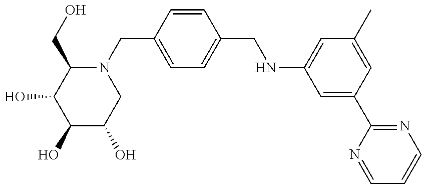 [SMILES: Cc1cc(NCc2ccc(CN3C[C@H](O)[C@@H](O)[C@H](O)[C@H]3CO)cc2)cc(-c2ncccn2)c1] |
| 1054 | 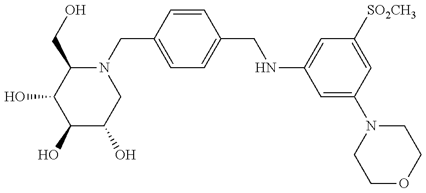 [SMILES: CS(=O)(=O)c1cc(NCc2ccc(CN3C[C@H](O)[C@@H](O)[C@H](O)[C@H]3CO)cc2)cc(N2CCOCC2)c1] |
| 1055 | 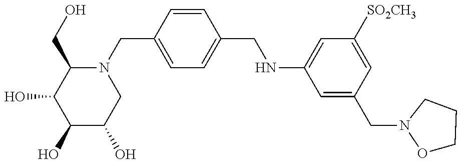 [SMILES: CS(=O)(=O)c1cc(CN2CCCO2)cc(NCc2ccc(CN3C[C@H](O)[C@@H](O)[C@H](O)[C@H]3CO)cc2)c1] |
| 1056 | 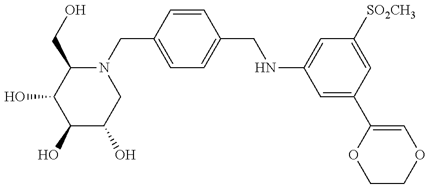 [SMILES: CS(=O)(=O)c1cc(NCc2ccc(CN3C[C@H](O)[C@@H](O)[C@H](O)[C@H]3CO)cc2)cc(C2=COCCO2)c1] |
| 1057 | 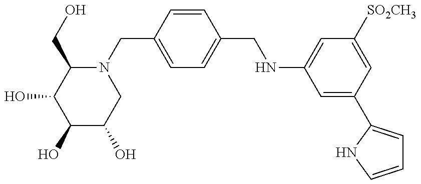 [SMILES: CS(=O)(=O)c1cc(NCc2ccc(CN3C[C@H](O)[C@@H](O)[C@H](O)[C@H]3CO)cc2)cc(-c2ccc[nH]2)c1] |
| 1058 | 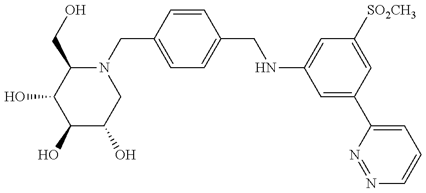 [SMILES: CS(=O)(=O)c1cc(NCc2ccc(CN3C[C@H](O)[C@@H](O)[C@H](O)[C@H]3CO)cc2)cc(-c2cccnn2)c1] |
| 1059 | 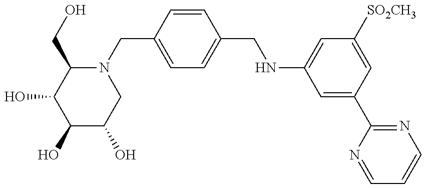 [SMILES: CS(=O)(=O)c1cc(NCc2ccc(CN3C[C@H](O)[C@@H](O)[C@H](O)[C@H]3CO)cc2)cc(-c2ncccn2)c1] |
| 1060 | 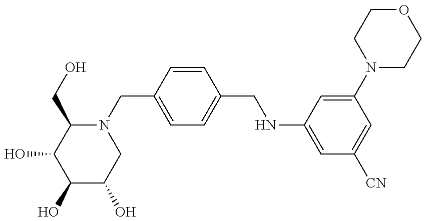 [SMILES: N#Cc1cc(NCc2ccc(CN3C[C@H](O)[C@@H](O)[C@H](O)[C@H]3CO)cc2)cc(N2CCOCC2)c1] |
| 1061 | 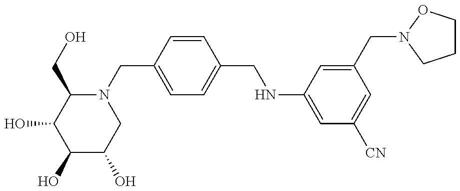 [SMILES: N#Cc1cc(CN2CCCO2)cc(NCc2ccc(CN3C[C@H](O)[C@@H](O)[C@H](O)[C@H]3CO)cc2)c1] |
| 1062 | 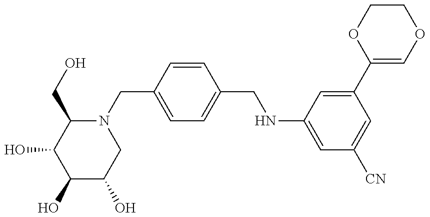 [SMILES: N#Cc1cc(NCc2ccc(CN3C[C@H](O)[C@@H](O)[C@H](O)[C@H]3CO)cc2)cc(C2=COCCO2)c1] |
| 1063 | 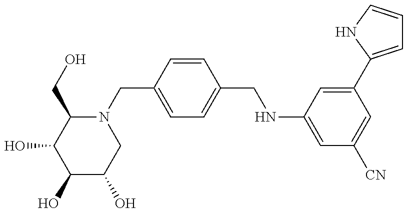 [SMILES: N#Cc1cc(NCc2ccc(CN3C[C@H](O)[C@@H](O)[C@H](O)[C@H]3CO)cc2)cc(-c2ccc[nH]2)c1] |
| 1064 | 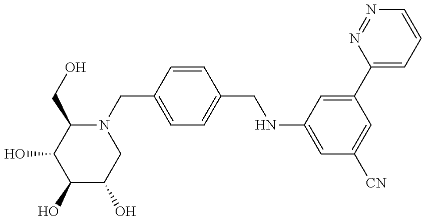 [SMILES: N#Cc1cc(NCc2ccc(CN3C[C@H](O)[C@@H](O)[C@H](O)[C@H]3CO)cc2)cc(-c2cccnn2)c1] |
| 1065 | 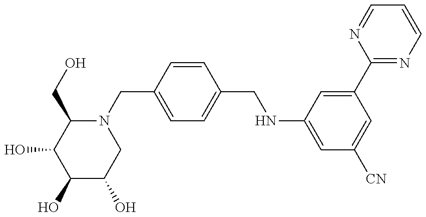 [SMILES: N#Cc1cc(NCc2ccc(CN3C[C@H](O)[C@@H](O)[C@H](O)[C@H]3CO)cc2)cc(-c2ncccn2)c1] |
| 1066 | 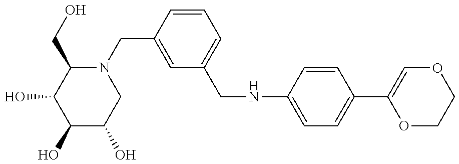 [SMILES: OC[C@@H]1[C@@H](O)[C@H](O)[C@@H](O)CN1Cc1cccc(CNc2ccc(C3=COCCO3)cc2)c1] |
| 1067 | 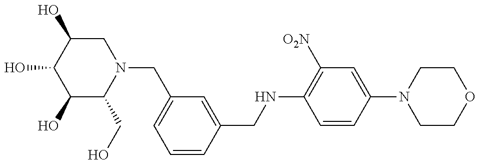 [SMILES: O=[N+]([O-])c1cc(N2CCOCC2)ccc1NCc1cccc(CN2C[C@H](O)[C@@H](O)[C@H](O)[C@H]2CO)c1] |
| 1068 | 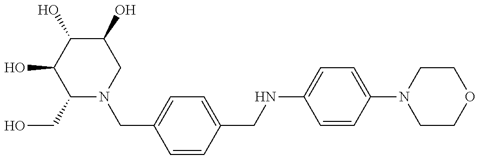 [SMILES: OC[C@@H]1[C@@H](O)[C@H](O)[C@@H](O)CN1Cc1ccc(CNc2ccc(N3CCOCC3)cc2)cc1] |
| 1069 | 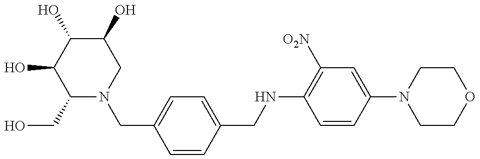 [SMILES: O=[N+]([O-])c1cc(N2CCOCC2)ccc1NCc1ccc(CN2C[C@H](O)[C@@H](O)[C@H](O)[C@H]2CO)cc1] |
| 1070 | 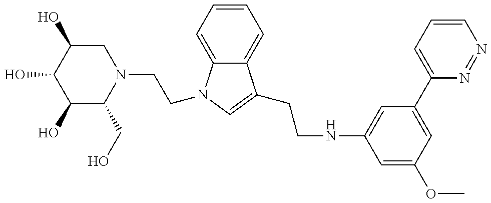 [SMILES: COc1cc(NCCc2cn(CCN3C[C@H](O)[C@@H](O)[C@H](O)[C@H]3CO)c3ccccc23)cc(-c2cccnn2)c1] |
| 1071 | 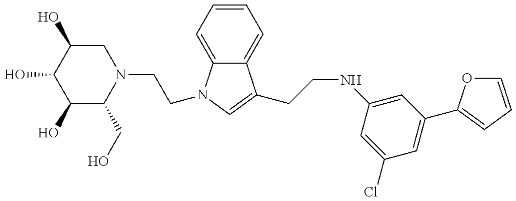 [SMILES: OC[C@@H]1[C@@H](O)[C@H](O)[C@@H](O)CN1CCn1cc(CCNc2cc(Cl)cc(-c3ccco3)c2)c2ccccc21] |
| 1072 | 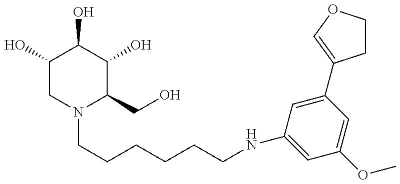 [SMILES: COc1cc(NCCCCCCN2C[C@H](O)[C@@H](O)[C@H](O)[C@H]2CO)cc(C2=COCC2)c1] |
| 1073 | 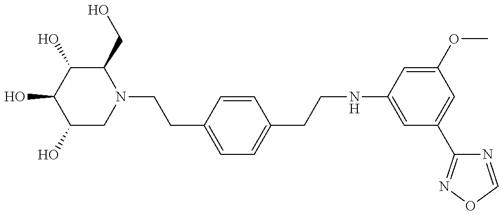 |
| 1074 | 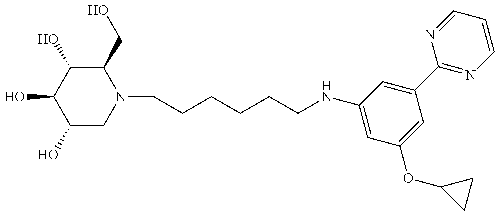 [SMILES: OC[C@@H]1[C@@H](O)[C@H](O)[C@@H](O)CN1CCCCCCNc1cc(OC2CC2)cc(-c2ncccn2)c1] |
| 1075 | 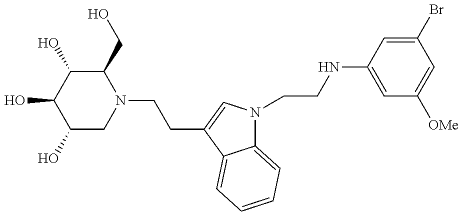 [SMILES: COc1cc(Br)cc(NCCn2cc(CCN3C[C@H](O)[C@@H](O)[C@H](O)[C@H]3CO)c3ccccc32)c1] |
| 1076 | 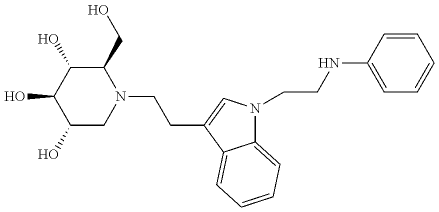 [SMILES: OC[C@@H]1[C@@H](O)[C@H](O)[C@@H](O)CN1CCc1cn(CCNc2ccccc2)c2ccccc12] |
| 1077 | 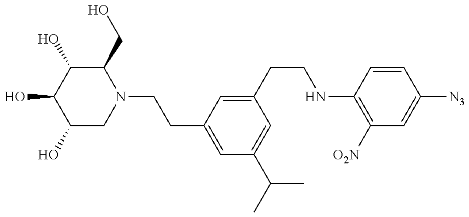 [SMILES: CC(C)c1cc(CCNc2ccc(N=[N+]=[N-])cc2[N+](=O)[O-])cc(CCN2C[C@H](O)[C@@H](O)[C@H](O)[C@H]2CO)c1] |
| 1078 | 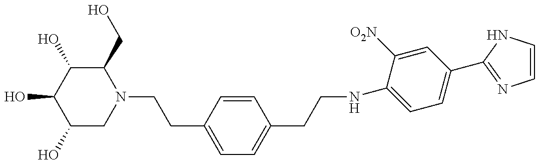 [SMILES: O=[N+]([O-])c1cc(-c2ncc[nH]2)ccc1NCCc1ccc(CCN2C[C@H](O)[C@@H](O)[C@H](O)[C@H]2CO)cc1] |
| 1079 | 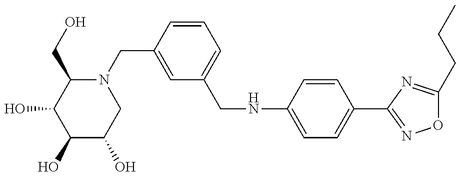 [SMILES: CCCc1nc(-c2ccc(NCc3cccc(CN4C[C@H](O)[C@@H](O)[C@H](O)[C@H]4CO)c3)cc2)no1] |
| 1080 | 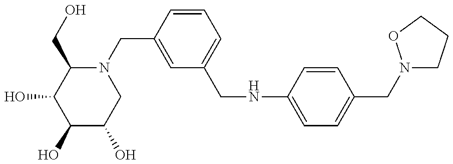 [SMILES: OC[C@@H]1[C@@H](O)[C@H](O)[C@@H](O)CN1Cc1cccc(CNc2ccc(CN3CCCO3)cc2)c1] |
| 1081 | 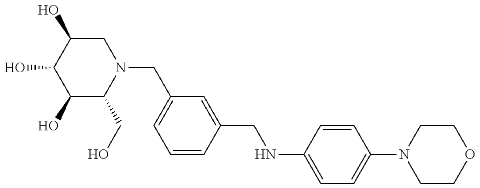 [SMILES: OC[C@@H]1[C@@H](O)[C@H](O)[C@@H](O)CN1Cc1cccc(CNc2ccc(N3CCOCC3)cc2)c1] |
| 1082 | 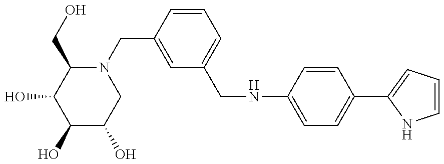 [SMILES: OC[C@@H]1[C@@H](O)[C@H](O)[C@@H](O)CN1Cc1cccc(CNc2ccc(-c3ccc[nH]3)cc2)c1] |
| 1083 | 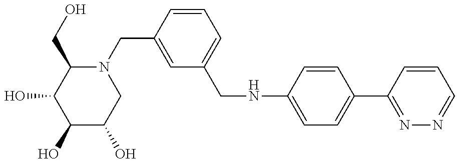 [SMILES: OC[C@@H]1[C@@H](O)[C@H](O)[C@@H](O)CN1Cc1cccc(CNc2ccc(-c3cccnn3)cc2)c1] |
| 1084 | 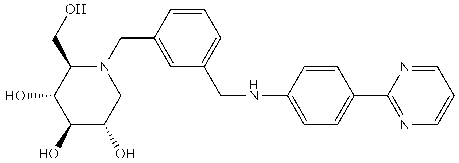 [SMILES: OC[C@@H]1[C@@H](O)[C@H](O)[C@@H](O)CN1Cc1cccc(CNc2ccc(-c3ncccn3)cc2)c1] |
| 1085 | 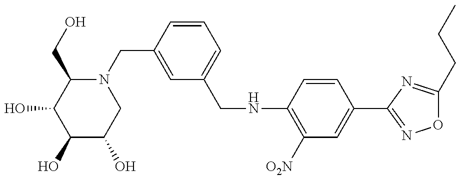 [SMILES: CCCc1nc(-c2ccc(NCc3cccc(CN4C[C@H](O)[C@@H](O)[C@H](O)[C@H]4CO)c3)c([N+](=O)[O-])c2)no1] |
| 1086 | 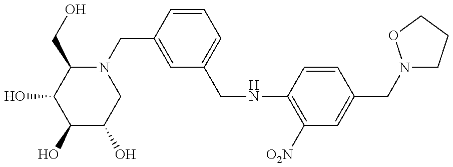 [SMILES: O=[N+]([O-])c1cc(CN2CCCO2)ccc1NCc1cccc(CN2C[C@H](O)[C@@H](O)[C@H](O)[C@H]2CO)c1] |
| 1087 | 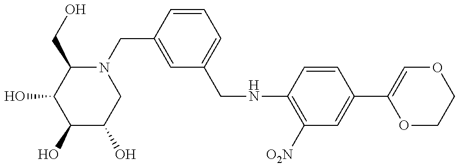 [SMILES: O=[N+]([O-])c1cc(C2=COCCO2)ccc1NCc1cccc(CN2C[C@H](O)[C@@H](O)[C@H](O)[C@H]2CO)c1] |
| 1088 | 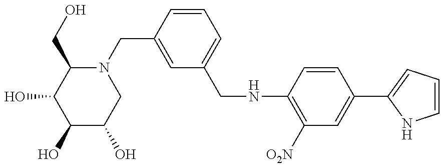 [SMILES: O=[N+]([O-])c1cc(-c2ccc[nH]2)ccc1NCc1cccc(CN2C[C@H](O)[C@@H](O)[C@H](O)[C@H]2CO)c1] |
| 1089 | 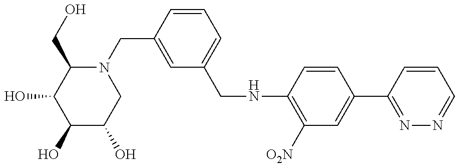 [SMILES: O=[N+]([O-])c1cc(-c2cccnn2)ccc1NCc1cccc(CN2C[C@H](O)[C@@H](O)[C@H](O)[C@H]2CO)c1] |
| 1090 | 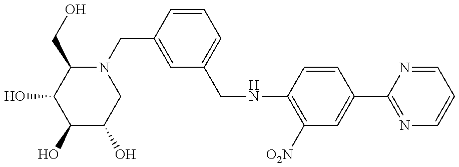 [SMILES: O=[N+]([O-])c1cc(-c2ncccn2)ccc1NCc1cccc(CN2C[C@H](O)[C@@H](O)[C@H](O)[C@H]2CO)c1] |
| 1091 | 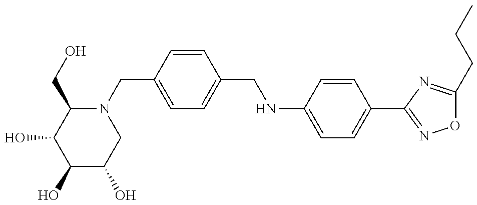 [SMILES: CCCc1nc(-c2ccc(NCc3ccc(CN4C[C@H](O)[C@@H](O)[C@H](O)[C@H]4CO)cc3)cc2)no1] |
| 1092 | 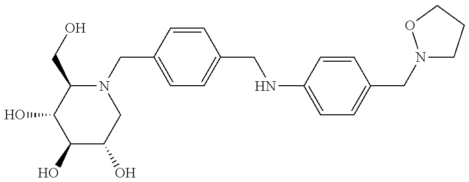 [SMILES: OC[C@@H]1[C@@H](O)[C@H](O)[C@@H](O)CN1Cc1ccc(CNc2ccc(CN3CCCO3)cc2)cc1] |
| 1093 | 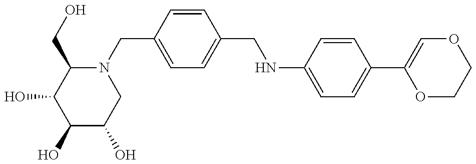 [SMILES: OC[C@@H]1[C@@H](O)[C@H](O)[C@@H](O)CN1Cc1ccc(CNc2ccc(C3=COCCO3)cc2)cc1] |
| 1094 | 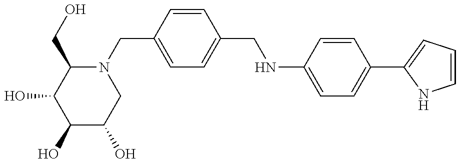 [SMILES: OC[C@@H]1[C@@H](O)[C@H](O)[C@@H](O)CN1Cc1ccc(CNc2ccc(-c3ccc[nH]3)cc2)cc1] |
| 1095 | 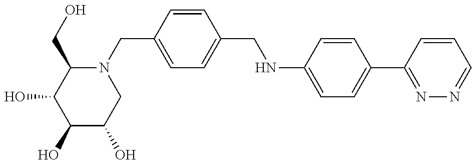 [SMILES: OC[C@@H]1[C@@H](O)[C@H](O)[C@@H](O)CN1Cc1ccc(CNc2ccc(-c3cccnn3)cc2)cc1] |
| 1096 | 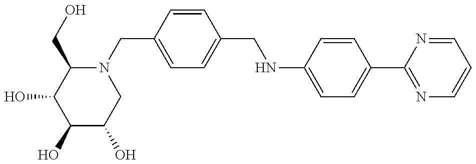 [SMILES: OC[C@@H]1[C@@H](O)[C@H](O)[C@@H](O)CN1Cc1ccc(CNc2ccc(-c3ncccn3)cc2)cc1] |
| 1097 | 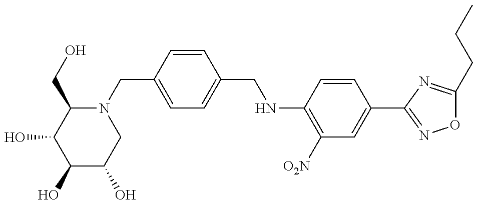 [SMILES: CCCc1nc(-c2ccc(NCc3ccc(CN4C[C@H](O)[C@@H](O)[C@H](O)[C@H]4CO)cc3)c([N+](=O)[O-])c2)no1] |
| 1098 | 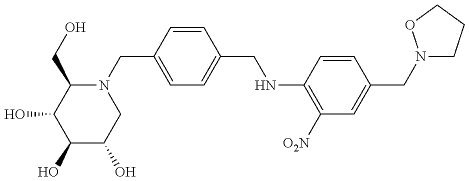 [SMILES: O=[N+]([O-])c1cc(CN2CCCO2)ccc1NCc1ccc(CN2C[C@H](O)[C@@H](O)[C@H](O)[C@H]2CO)cc1] |
| 1099 | 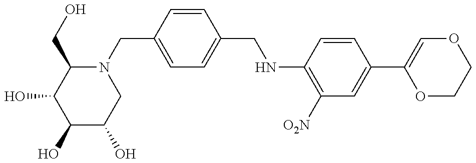 [SMILES: O=[N+]([O-])c1cc(C2=COCCO2)ccc1NCc1ccc(CN2C[C@H](O)[C@@H](O)[C@H](O)[C@H]2CO)cc1] |
| 1100 | 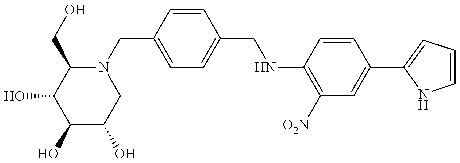 [SMILES: O=[N+]([O-])c1cc(-c2ccc[nH]2)ccc1NCc1ccc(CN2C[C@H](O)[C@@H](O)[C@H](O)[C@H]2CO)cc1] |
| 1101 | 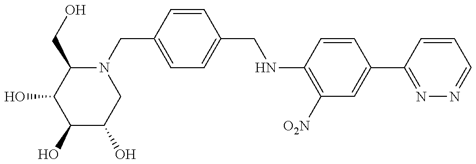 [SMILES: O=[N+]([O-])c1cc(-c2cccnn2)ccc1NCc1ccc(CN2C[C@H](O)[C@@H](O)[C@H](O)[C@H]2CO)cc1] |
| 1102 | 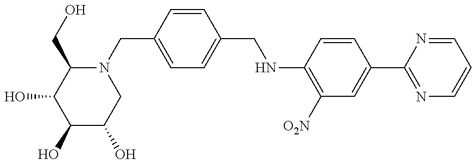 [SMILES: O=[N+]([O-])c1cc(-c2ncccn2)ccc1NCc1ccc(CN2C[C@H](O)[C@@H](O)[C@H](O)[C@H]2CO)cc1] |
| 1103 | 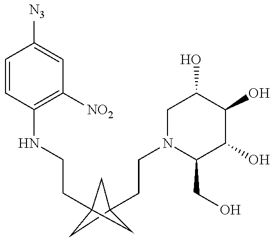 [SMILES: [N-]=[N+]=Nc1ccc(NCCC23CC(CCN4C[C@H](O)[C@@H](O)[C@H](O)[C@H]4CO)(C2)C3)c([N+](=O)[O-])c1] |
| 1104 | 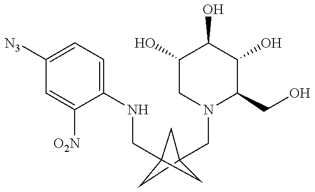 [SMILES: [N-]=[N+]=Nc1ccc(NCC23CC(CN4C[C@H](O)[C@@H](O)[C@H](O)[C@H]4CO)(C2)C3)c([N+](=O)[O-])c1] |
| 1105 | 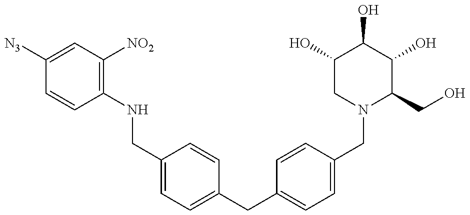 [SMILES: [N-]=[N+]=Nc1ccc(NCc2ccc(Cc3ccc(CN4C[C@H](O)[C@@H](O)[C@H](O)[C@H]4CO)cc3)cc2)c([N+](=O)[O-])c1] |
| 1106 | 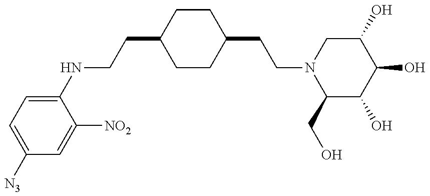 |
| 1107 | 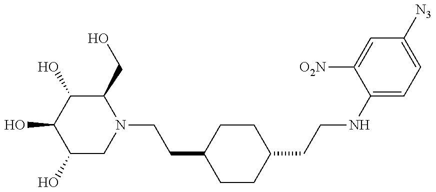 |
| 1108 | 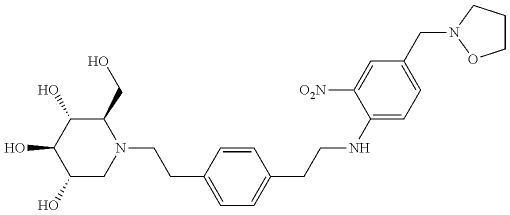 [SMILES: O=[N+]([O-])c1cc(CN2CCCO2)ccc1NCCc1ccc(CCN2C[C@H](O)[C@@H](O)[C@H](O)[C@H]2CO)cc1] |
| 1109 | 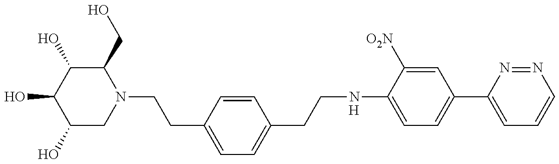 |
| 1110 | 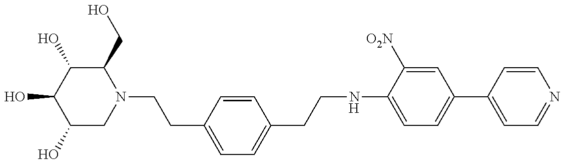 |
| 1111 | 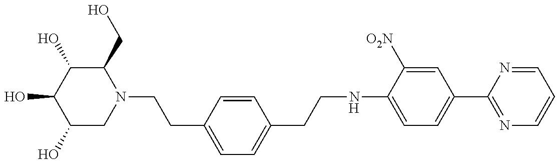 |
| 1112 | 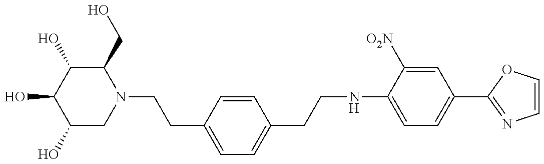 |
| 1113 | 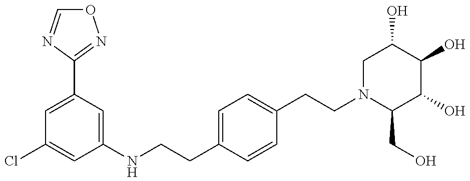 [SMILES: OC[C@@H]1[C@@H](O)[C@H](O)[C@@H](O)CN1CCc1ccc(CCNc2cc(Cl)cc(-c3ncon3)c2)cc1] |
| 1114 | 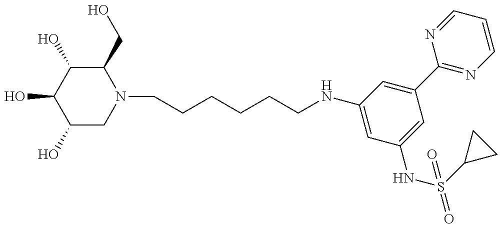 [SMILES: O=S(=O)(Nc1cc(NCCCCCCN2C[C@H](O)[C@@H](O)[C@H](O)[C@H]2CO)cc(-c2ncccn2)c1)C1CC1] |
| 1115 | 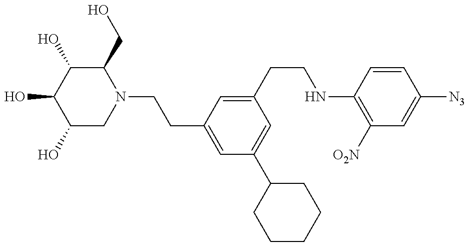 [SMILES: [N-]=[N+]=Nc1ccc(NCCc2cc(CCN3C[C@H](O)[C@@H](O)[C@H](O)[C@H]3CO)cc(C3CCCCC3)c2)c([N+](=O)[O-])c1] |
| 1116 | 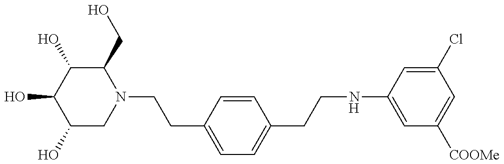 |
| 1117 | 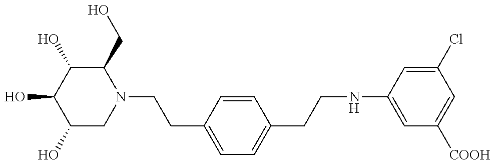 |
| 1118 | 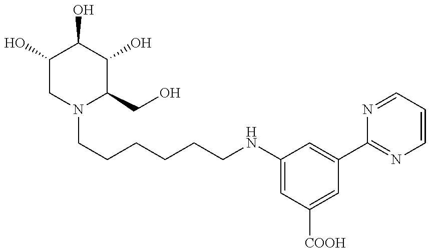 [SMILES: O=C(O)c1cc(NCCCCCCN2C[C@H](O)[C@@H](O)[C@H](O)[C@H]2CO)cc(-c2ncccn2)c1] |
| 1119 | 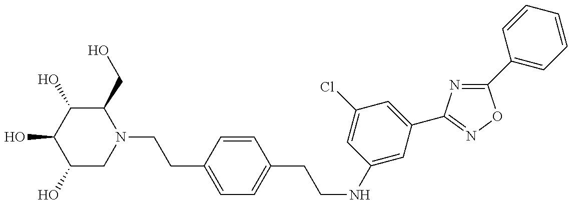 [SMILES: OC[C@@H]1[C@@H](O)[C@H](O)[C@@H](O)CN1CCc1ccc(CCNc2cc(Cl)cc(-c3noc(-c4ccccc4)n3)c2)cc1] |
| 1120 | 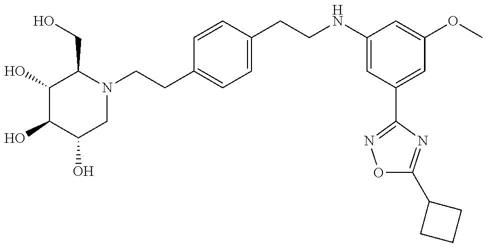 [SMILES: COc1cc(NCCc2ccc(CCN3C[C@H](O)[C@@H](O)[C@H](O)[C@H]3CO)cc2)cc(-c2noc(C3CCC3)n2)c1] |
| 1121 | |
| 1122 | 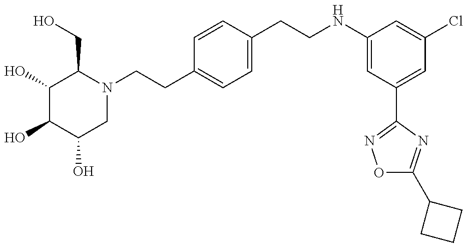 [SMILES: OC[C@@H]1[C@@H](O)[C@H](O)[C@@H](O)CN1CCc1ccc(CCNc2cc(Cl)cc(-c3noc(C4CCC4)n3)c2)cc1] |
| 1123 | 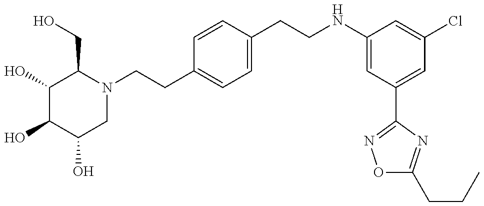 [SMILES: CCCc1nc(-c2cc(Cl)cc(NCCc3ccc(CCN4C[C@H](O)[C@@H](O)[C@H](O)[C@H]4CO)cc3)c2)no1] |
| 1124 | 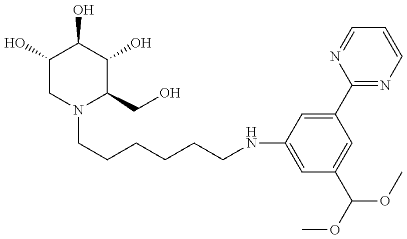 [SMILES: COC(OC)c1cc(NCCCCCCN2C[C@H](O)[C@@H](O)[C@H](O)[C@H]2CO)cc(-c2ncccn2)c1] |
| 1125 | 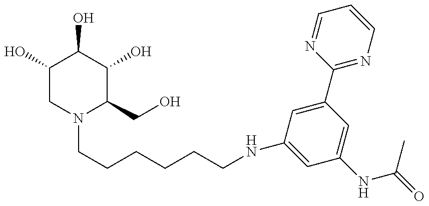 [SMILES: CC(=O)Nc1cc(NCCCCCCN2C[C@H](O)[C@@H](O)[C@H](O)[C@H]2CO)cc(-c2ncccn2)c1] |
| 1126 | 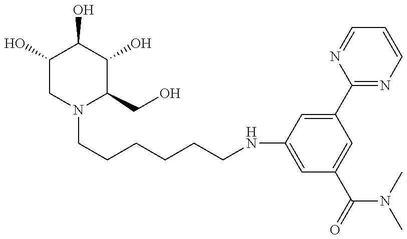 [SMILES: CN(C)C(=O)c1cc(NCCCCCCN2C[C@H](O)[C@@H](O)[C@H](O)[C@H]2CO)cc(-c2ncccn2)c1] |
| 1127 | 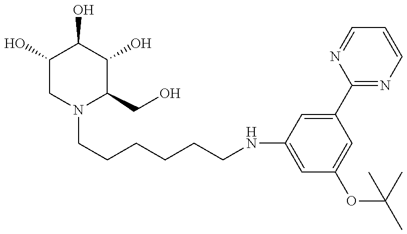 [SMILES: CC(C)(C)Oc1cc(NCCCCCCN2C[C@H](O)[C@@H](O)[C@H](O)[C@H]2CO)cc(-c2ncccn2)c1] |
| 1128 | 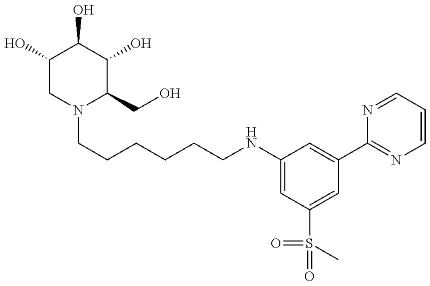 [SMILES: CS(=O)(=O)c1cc(NCCCCCCN2C[C@H](O)[C@@H](O)[C@H](O)[C@H]2CO)cc(-c2ncccn2)c1] |
| 1129 | 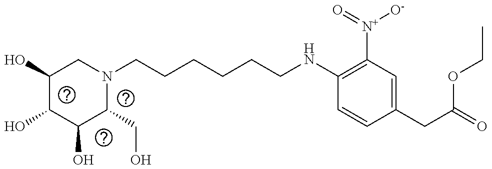 [SMILES: CCOC(=O)Cc1ccc(NCCCCCCN2C[C@H](O)[C@@H](O)[C@H](O)[C@H]2CO)c([N+](=O)[O-])c1] |
| 1130 | 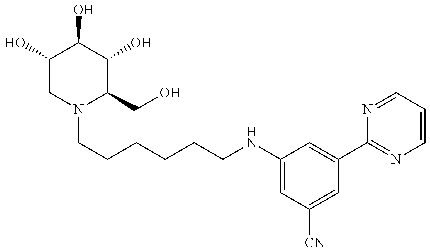 [SMILES: N#Cc1cc(NCCCCCCN2C[C@H](O)[C@@H](O)[C@H](O)[C@H]2CO)cc(-c2ncccn2)c1] |
| 1131 | 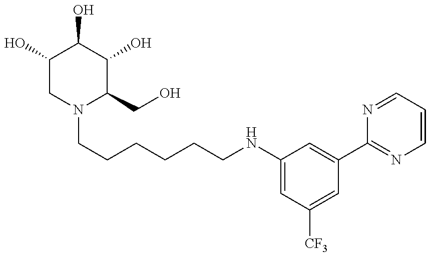 [SMILES: OC[C@@H]1[C@@H](O)[C@H](O)[C@@H](O)CN1CCCCCCNc1cc(-c2ncccn2)cc(C(F)(F)F)c1] |
| 1132 | 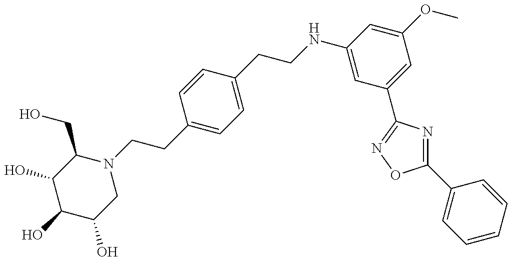 [SMILES: COc1cc(NCCc2ccc(CCN3C[C@H](O)[C@@H](O)[C@H](O)[C@H]3CO)cc2)cc(-c2noc(-c3ccccc3)n2)c1] |
| 1133 | 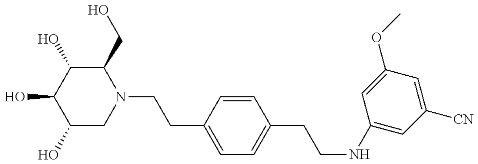 [SMILES: COc1cc(C#N)cc(NCCc2ccc(CCN3C[C@H](O)[C@@H](O)[C@H](O)[C@H]3CO)cc2)c1] |
| 1134 | 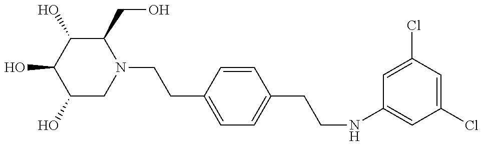 [SMILES: OC[C@@H]1[C@@H](O)[C@H](O)[C@@H](O)CN1CCc1ccc(CCNc2cc(Cl)cc(Cl)c2)cc1] |
| 1135 | 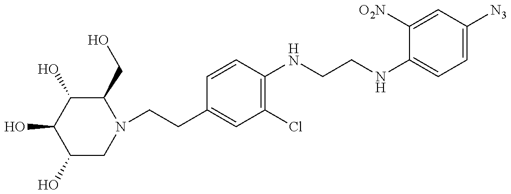 [SMILES: [N-]=[N+]=Nc1ccc(NCCNc2ccc(CCN3C[C@H](O)[C@@H](O)[C@H](O)[C@H]3CO)cc2Cl)c([N+](=O)[O-])c1] |
| 1136 | 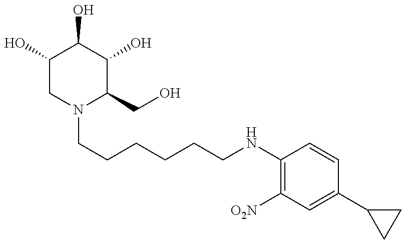 [SMILES: O=[N+]([O-])c1cc(C2CC2)ccc1NCCCCCCN1C[C@H](O)[C@@H](O)[C@H](O)[C@H]1CO] |
| 1137 | 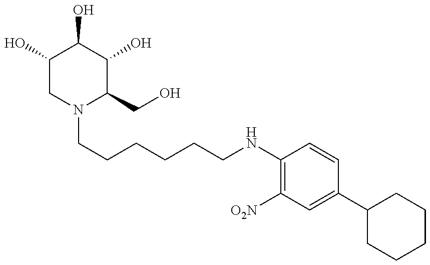 [SMILES: O=[N+]([O-])c1cc(C2CCCCC2)ccc1NCCCCCCN1C[C@H](O)[C@@H](O)[C@H](O)[C@H]1CO] |
| 1138 | 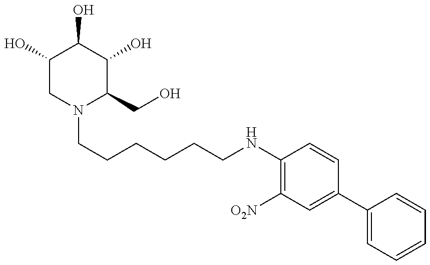 [SMILES: O=[N+]([O-])c1cc(-c2ccccc2)ccc1NCCCCCCN1C[C@H](O)[C@@H](O)[C@H](O)[C@H]1CO] |
| 1139 | 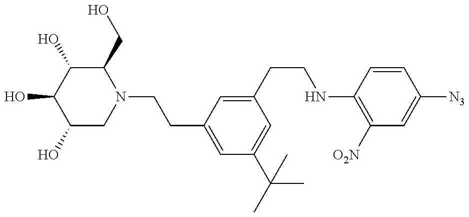 [SMILES: CC(C)(C)c1cc(CCNc2ccc(N=[N+]=[N-])cc2[N+](=O)[O-])cc(CCN2C[C@H](O)[C@@H](O)[C@H](O)[C@H]2CO)c1] |
| 1140 | 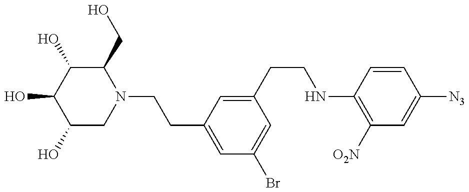 [SMILES: [N-]=[N+]=Nc1ccc(NCCc2cc(Br)cc(CCN3C[C@H](O)[C@@H](O)[C@H](O)[C@H]3CO)c2)c([N+](=O)[O-])c1] |
| 1141 | 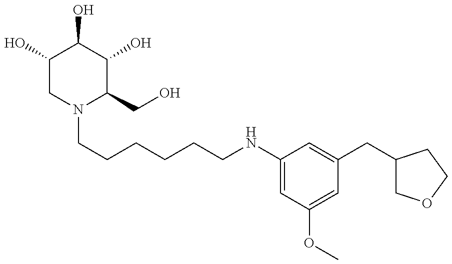 [SMILES: COc1cc(CC2CCOC2)cc(NCCCCCCN2C[C@H](O)[C@@H](O)[C@H](O)[C@H]2CO)c1] |
| 1142 | 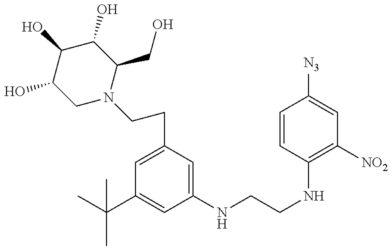 [SMILES: CC(C)(C)c1cc(CCN2C[C@H](O)[C@@H](O)[C@H](O)[C@H]2CO)cc(NCCNc2ccc(N=[N+]=[N-])cc2[N+](=O)[O-])c1] |
| 1143 | 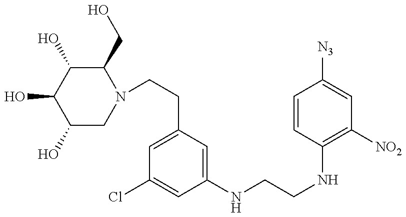 [SMILES: [N-]=[N+]=Nc1ccc(NCCNc2cc(Cl)cc(CCN3C[C@H](O)[C@@H](O)[C@H](O)[C@H]3CO)c2)c([N+](=O)[O-])c1] |
| 1144 | 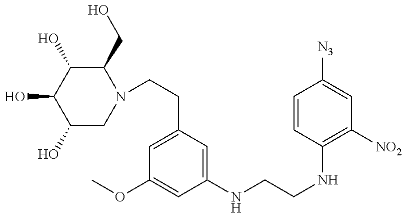 [SMILES: COc1cc(CCN2C[C@H](O)[C@@H](O)[C@H](O)[C@H]2CO)cc(NCCNc2ccc(N=[N+]=[N-])cc2[N+](=O)[O-])c1] |
| 1145 | 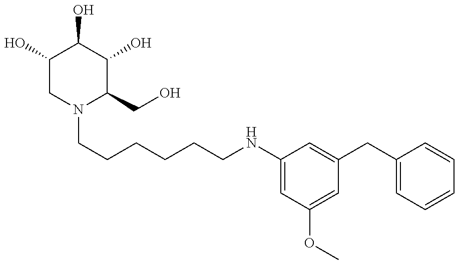 [SMILES: COc1cc(Cc2ccccc2)cc(NCCCCCCN2C[C@H](O)[C@@H](O)[C@H](O)[C@H]2CO)c1] |
| 1146 | 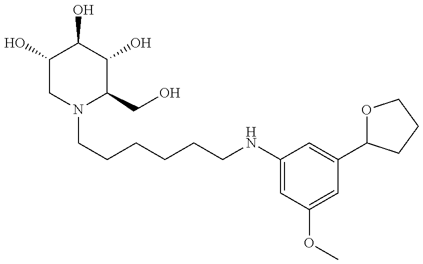 [SMILES: COc1cc(NCCCCCCN2C[C@H](O)[C@@H](O)[C@H](O)[C@H]2CO)cc(C2CCCO2)c1] |
| 1147 | 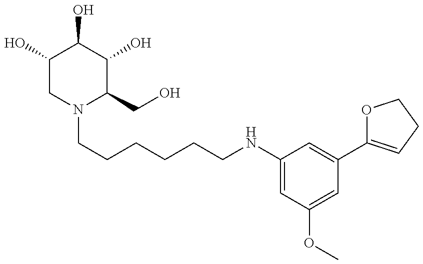 [SMILES: COc1cc(NCCCCCCN2C[C@H](O)[C@@H](O)[C@H](O)[C@H]2CO)cc(C2=CCCO2)c1] |
| 1148 | 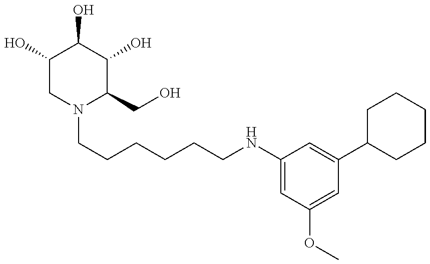 [SMILES: COc1cc(NCCCCCCN2C[C@H](O)[C@@H](O)[C@H](O)[C@H]2CO)cc(C2CCCCC2)c1] |
| 1149 | 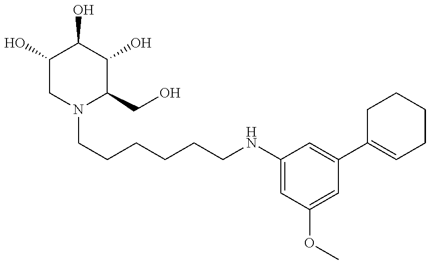 [SMILES: COc1cc(NCCCCCCN2C[C@H](O)[C@@H](O)[C@H](O)[C@H]2CO)cc(C2=CCCCC2)c1] |
| 1150 | 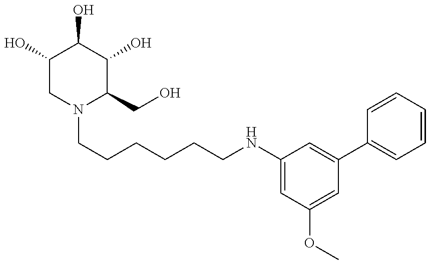 [SMILES: COc1cc(NCCCCCCN2C[C@H](O)[C@@H](O)[C@H](O)[C@H]2CO)cc(-c2ccccc2)c1] |
| 1151 | 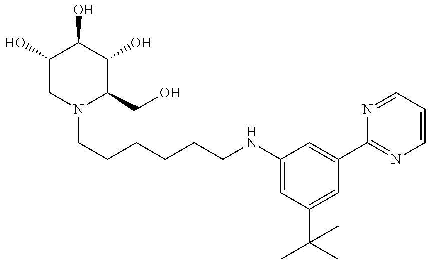 [SMILES: CC(C)(C)c1cc(NCCCCCCN2C[C@H](O)[C@@H](O)[C@H](O)[C@H]2CO)cc(-c2ncccn2)c1] |
| 1152 | 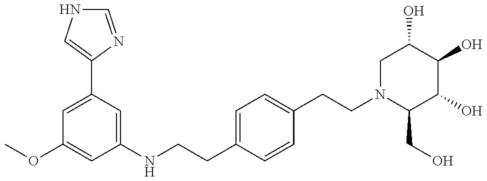 [SMILES: COc1cc(NCCc2ccc(CCN3C[C@H](O)[C@@H](O)[C@H](O)[C@H]3CO)cc2)cc(-c2c[nH]cn2)c1] |
| 1153 | 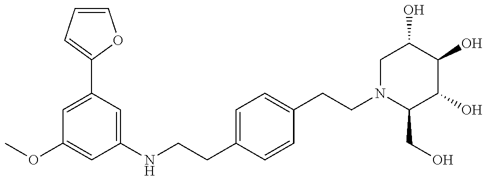 [SMILES: COc1cc(NCCc2ccc(CCN3C[C@H](O)[C@@H](O)[C@H](O)[C@H]3CO)cc2)cc(-c2ccco2)c1] |
| 1154 | 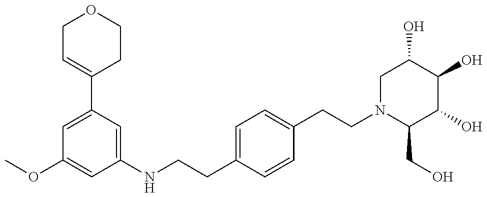 [SMILES: COc1cc(NCCc2ccc(CCN3C[C@H](O)[C@@H](O)[C@H](O)[C@H]3CO)cc2)cc(C2=CCOCC2)c1] |
| 1155 | 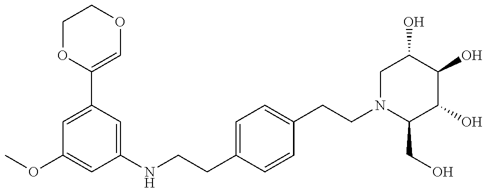 [SMILES: COc1cc(NCCc2ccc(CCN3C[C@H](O)[C@@H](O)[C@H](O)[C@H]3CO)cc2)cc(C2=COCCO2)c1] |
| 1156 | 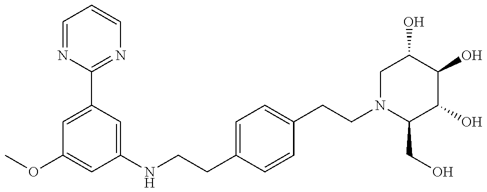 [SMILES: COc1cc(NCCc2ccc(CCN3C[C@H](O)[C@@H](O)[C@H](O)[C@H]3CO)cc2)cc(-c2ncccn2)c1] |
| 1157 | 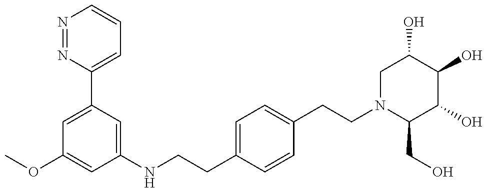 [SMILES: COc1cc(NCCc2ccc(CCN3C[C@H](O)[C@@H](O)[C@H](O)[C@H]3CO)cc2)cc(-c2cccnn2)c1] |
| 1158 | 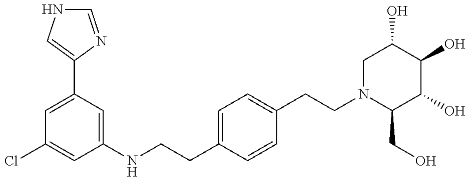 [SMILES: OC[C@@H]1[C@@H](O)[C@H](O)[C@@H](O)CN1CCc1ccc(CCNc2cc(Cl)cc(-c3c[nH]cn3)c2)cc1] |
| 1159 | 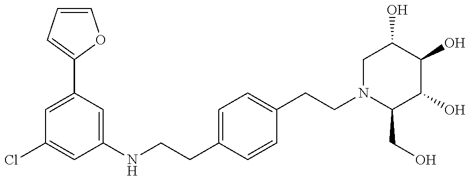 [SMILES: OC[C@@H]1[C@@H](O)[C@H](O)[C@@H](O)CN1CCc1ccc(CCNc2cc(Cl)cc(-c3ccco3)c2)cc1] |
| 1160 | 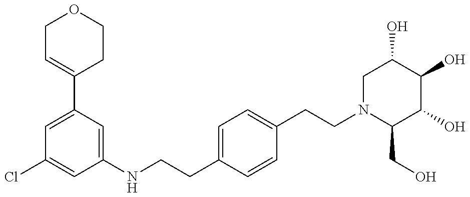 [SMILES: OC[C@@H]1[C@@H](O)[C@H](O)[C@@H](O)CN1CCc1ccc(CCNc2cc(Cl)cc(C3=CCOCC3)c2)cc1] |
| 1161 | 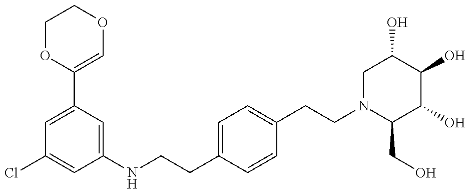 [SMILES: OC[C@@H]1[C@@H](O)[C@H](O)[C@@H](O)CN1CCc1ccc(CCNc2cc(Cl)cc(C3=COCCO3)c2)cc1] |
| 1162 | 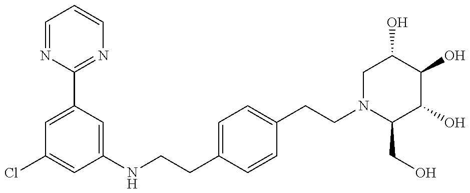 [SMILES: OC[C@@H]1[C@@H](O)[C@H](O)[C@@H](O)CN1CCc1ccc(CCNc2cc(Cl)cc(-c3ncccn3)c2)cc1] |
| 1163 | 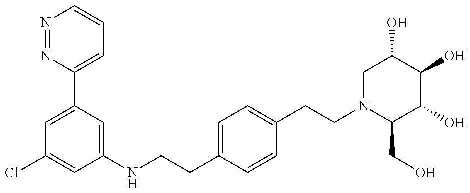 [SMILES: OC[C@@H]1[C@@H](O)[C@H](O)[C@@H](O)CN1CCc1ccc(CCNc2cc(Cl)cc(-c3cccnn3)c2)cc1] |
| 1164 | 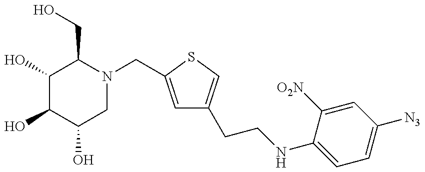 [SMILES: [N-]=[N+]=Nc1ccc(NCCc2csc(CN3C[C@H](O)[C@@H](O)[C@H](O)[C@H]3CO)c2)c([N+](=O)[O-])c1] |
| 1165 | 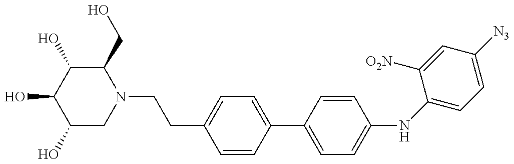 [SMILES: [N-]=[N+]=Nc1ccc(Nc2ccc(-c3ccc(CCN4C[C@H](O)[C@@H](O)[C@H](O)[C@H]4CO)cc3)cc2)c([N+](=O)[O-])c1] |
| 1166 | 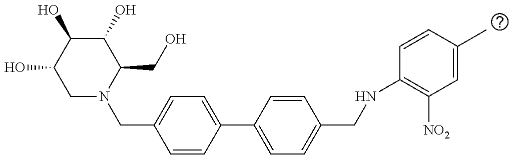 [SMILES: Cc1ccc(NCc2ccc(-c3ccc(CN4C[C@H](O)[C@@H](O)[C@H](O)[C@H]4CO)cc3)cc2)c([N+](=O)[O-])c1] |
| 1167 | 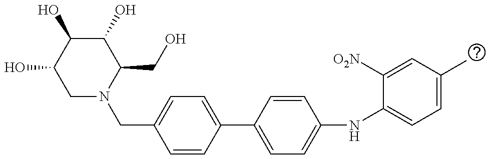 [SMILES: Cc1ccc(Nc2ccc(-c3ccc(CN4C[C@H](O)[C@@H](O)[C@H](O)[C@H]4CO)cc3)cc2)c([N+](=O)[O-])c1] |
| 1168 | 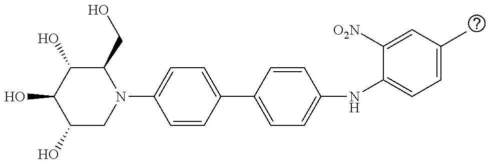 [SMILES: Cc1ccc(Nc2ccc(-c3ccc(N4C[C@H](O)[C@@H](O)[C@H](O)[C@H]4CO)cc3)cc2)c([N+](=O)[O-])c1] |
| 1169 | 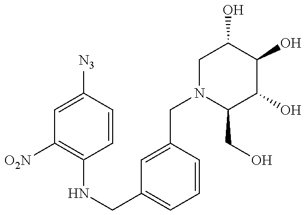 [SMILES: [N-]=[N+]=Nc1ccc(NCc2cccc(CN3C[C@H](O)[C@@H](O)[C@H](O)[C@H]3CO)c2)c([N+](=O)[O-])c1] |
| 1170 | 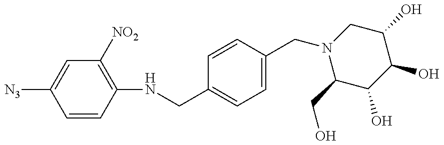 [SMILES: [N-]=[N+]=Nc1ccc(NCc2ccc(CN3C[C@H](O)[C@@H](O)[C@H](O)[C@H]3CO)cc2)c([N+](=O)[O-])c1] |
| 1171 | 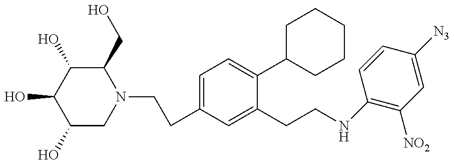 [SMILES: [N-]=[N+]=Nc1ccc(NCCc2cc(CCN3C[C@H](O)[C@@H](O)[C@H](O)[C@H]3CO)ccc2C2CCCCC2)c([N+](=O)[O-])c1] |
| 1172 | 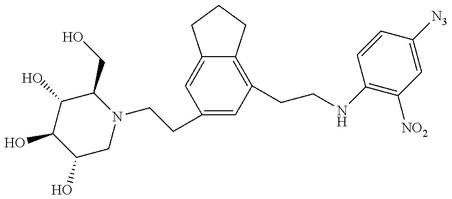 [SMILES: [N-]=[N+]=Nc1ccc(NCCc2cc(CCN3C[C@H](O)[C@@H](O)[C@H](O)[C@H]3CO)cc3c2CCC3)c([N+](=O)[O-])c1] |
| 1173 | 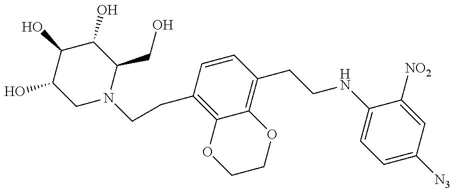 [SMILES: [N-]=[N+]=Nc1ccc(NCCc2ccc(CCN3C[C@H](O)[C@@H](O)[C@H](O)[C@H]3CO)c3c2OCCO3)c([N+](=O)[O-])c1] |
| 1174 | 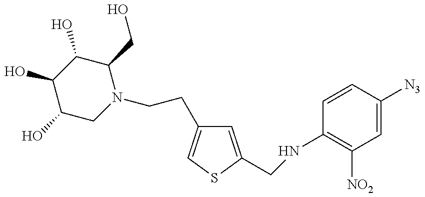 [SMILES: [N-]=[N+]=Nc1ccc(NCc2cc(CCN3C[C@H](O)[C@@H](O)[C@H](O)[C@H]3CO)cs2)c([N+](=O)[O-])c1] |
| 1175 | 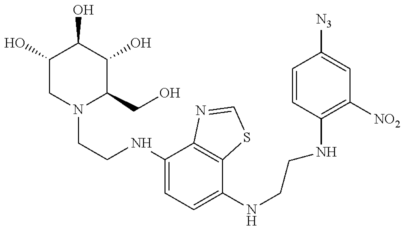 [SMILES: [N-]=[N+]=Nc1ccc(NCCNc2ccc(NCCN3C[C@H](O)[C@@H](O)[C@H](O)[C@H]3CO)c3ncsc23)c([N+](=O)[O-])c1] |
| 1176 | 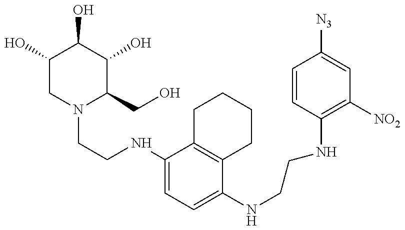 [SMILES: [N-]=[N+]=Nc1ccc(NCCNc2ccc(NCCN3C[C@H](O)[C@@H](O)[C@H](O)[C@H]3CO)c3c2CCCC3)c([N+](=O)[O-])c1] |
| 1177 | 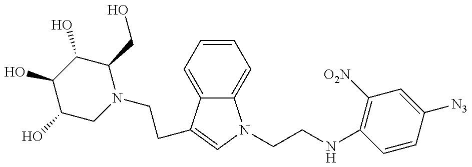 [SMILES: [N-]=[N+]=Nc1ccc(NCCn2cc(CCN3C[C@H](O)[C@@H](O)[C@H](O)[C@H]3CO)c3ccccc32)c([N+](=O)[O-])c1] |
| 1178 | 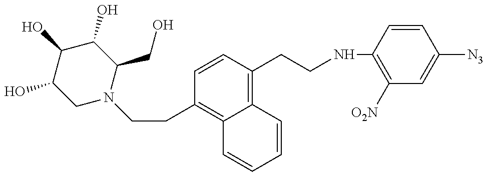 [SMILES: [N-]=[N+]=Nc1ccc(NCCc2ccc(CCN3C[C@H](O)[C@@H](O)[C@H](O)[C@H]3CO)c3ccccc23)c([N+](=O)[O-])c1] |
| 1179 | 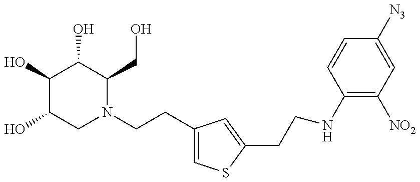 [SMILES: [N-]=[N+]=Nc1ccc(NCCc2cc(CCN3C[C@H](O)[C@@H](O)[C@H](O)[C@H]3CO)cs2)c([N+](=O)[O-])c1] |
| 1180 | 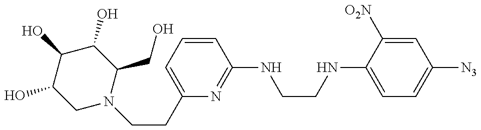 [SMILES: [N-]=[N+]=Nc1ccc(NCCNc2cccc(CCN3C[C@H](O)[C@@H](O)[C@H](O)[C@H]3CO)n2)c([N+](=O)[O-])c1] |
| 1181 | 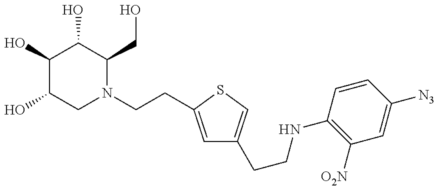 [SMILES: [N-]=[N+]=Nc1ccc(NCCc2csc(CCN3C[C@H](O)[C@@H](O)[C@H](O)[C@H]3CO)c2)c([N+](=O)[O-])c1] |
| 1182 | 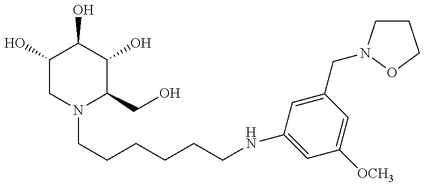 [SMILES: COc1cc(CN2CCCO2)cc(NCCCCCCN2C[C@H](O)[C@@H](O)[C@H](O)[C@H]2CO)c1] |
| 1183 | 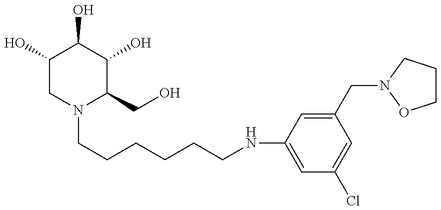 [SMILES: OC[C@@H]1[C@@H](O)[C@H](O)[C@@H](O)CN1CCCCCCNc1cc(Cl)cc(CN2CCCO2)c1] |
| 1184 | 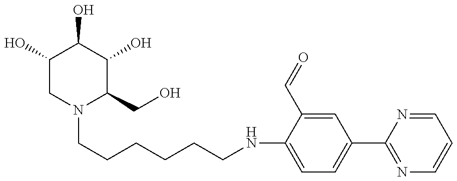 [SMILES: O=Cc1cc(-c2ncccn2)ccc1NCCCCCCN1C[C@H](O)[C@@H](O)[C@H](O)[C@H]1CO] |
| 1185 | 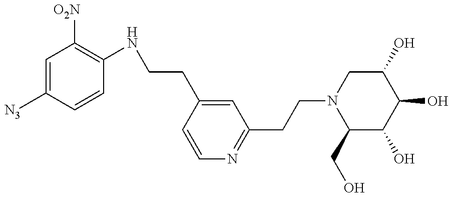 [SMILES: [N-]=[N+]=Nc1ccc(NCCc2ccnc(CCN3C[C@H](O)[C@@H](O)[C@H](O)[C@H]3CO)c2)c([N+](=O)[O-])c1] |
| 1186 | 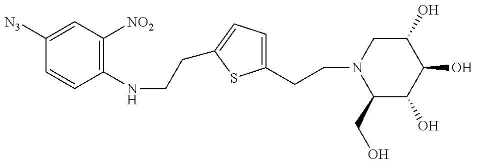 [SMILES: [N-]=[N+]=Nc1ccc(NCCc2ccc(CCN3C[C@H](O)[C@@H](O)[C@H](O)[C@H]3CO)s2)c([N+](=O)[O-])c1] |
| 1187 | 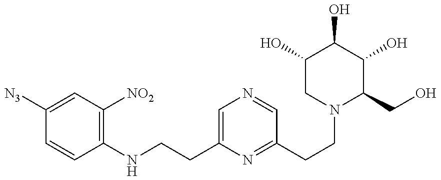 [SMILES: [N-]=[N+]=Nc1ccc(NCCc2cncc(CCN3C[C@H](O)[C@@H](O)[C@H](O)[C@H]3CO)n2)c([N+](=O)[O-])c1] |
| 1188 | 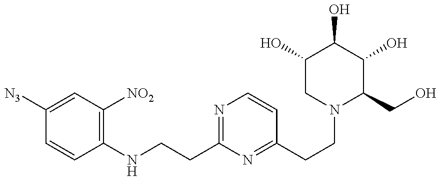 [SMILES: [N-]=[N+]=Nc1ccc(NCCc2nccc(CCN3C[C@H](O)[C@@H](O)[C@H](O)[C@H]3CO)n2)c([N+](=O)[O-])c1] |
| 1189 | 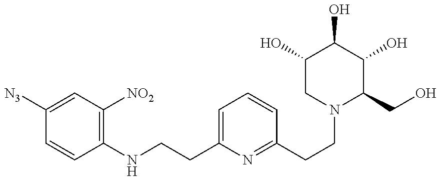 [SMILES: [N-]=[N+]=Nc1ccc(NCCc2cccc(CCN3C[C@H](O)[C@@H](O)[C@H](O)[C@H]3CO)n2)c([N+](=O)[O-])c1] |
| 1190 | 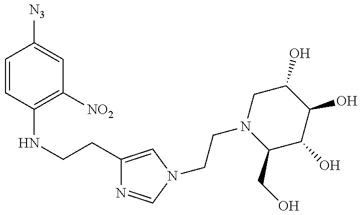 [SMILES: [N-]=[N+]=Nc1ccc(NCCc2cn(CCN3C[C@H](O)[C@@H](O)[C@H](O)[C@H]3CO)cn2)c([N+](=O)[O-])c1] |
| 1191 | 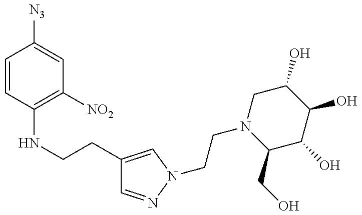 [SMILES: [N-]=[N+]=Nc1ccc(NCCc2cnn(CCN3C[C@H](O)[C@@H](O)[C@H](O)[C@H]3CO)c2)c([N+](=O)[O-])c1] |
| 1192 | 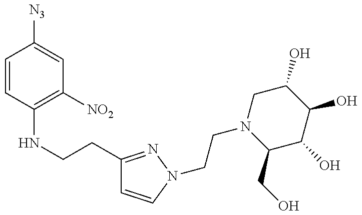 [SMILES: [N-]=[N+]=Nc1ccc(NCCc2ccn(CCN3C[C@H](O)[C@@H](O)[C@H](O)[C@H]3CO)n2)c([N+](=O)[O-])c1] |
| 1193 | 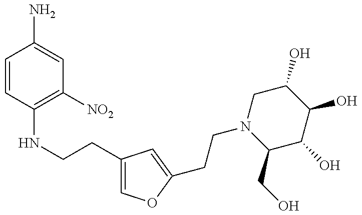 [SMILES: Nc1ccc(NCCc2coc(CCN3C[C@H](O)[C@@H](O)[C@H](O)[C@H]3CO)c2)c([N+](=O)[O-])c1] |
| 1194 | 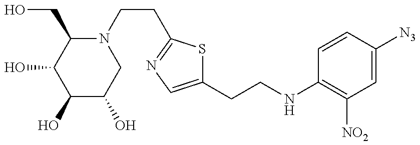 [SMILES: [N-]=[N+]=Nc1ccc(NCCc2cnc(CCN3C[C@H](O)[C@@H](O)[C@H](O)[C@H]3CO)s2)c([N+](=O)[O-])c1] |
| 1195 | 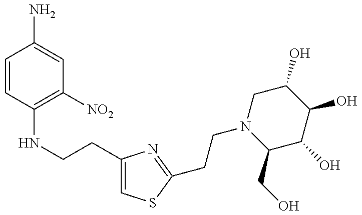 [SMILES: Nc1ccc(NCCc2csc(CCN3C[C@H](O)[C@@H](O)[C@H](O)[C@H]3CO)n2)c([N+](=O)[O-])c1] |
| 1196 | 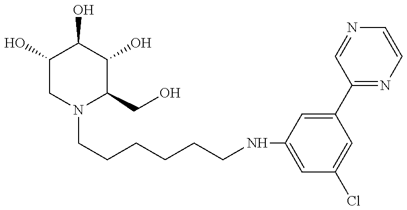 [SMILES: OC[C@@H]1[C@@H](O)[C@H](O)[C@@H](O)CN1CCCCCCNc1cc(Cl)cc(-c2cnccn2)c1] |
| 1197 | 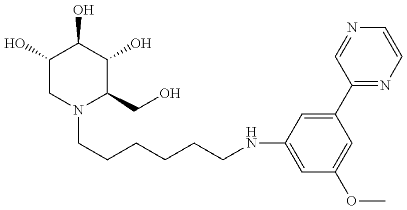 [SMILES: COc1cc(NCCCCCCN2C[C@H](O)[C@@H](O)[C@H](O)[C@H]2CO)cc(-c2cnccn2)c1] |
| 1198 | 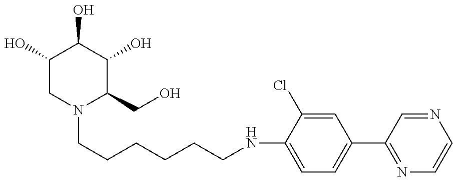 [SMILES: OC[C@@H]1[C@@H](O)[C@H](O)[C@@H](O)CN1CCCCCCNc1ccc(-c2cnccn2)cc1Cl] |
| 1199 | 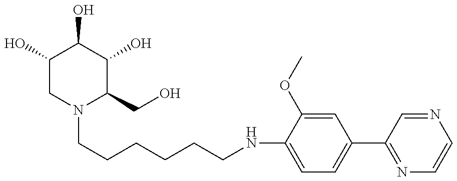 [SMILES: COc1cc(-c2cnccn2)ccc1NCCCCCCN1C[C@H](O)[C@@H](O)[C@H](O)[C@H]1CO] |
| 1200 | 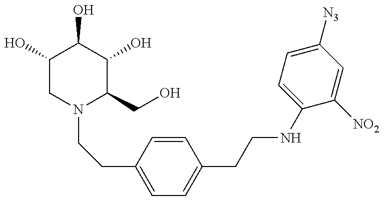 [SMILES: [N-]=[N+]=Nc1ccc(NCCc2ccc(CCN3C[C@H](O)[C@@H](O)[C@H](O)[C@H]3CO)cc2)c([N+](=O)[O-])c1] |
| 1201 | 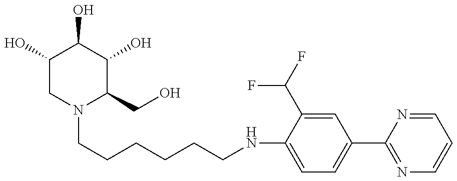 [SMILES: OC[C@@H]1[C@@H](O)[C@H](O)[C@@H](O)CN1CCCCCCNc1ccc(-c2ncccn2)cc1C(F)F] |
| 1202 | 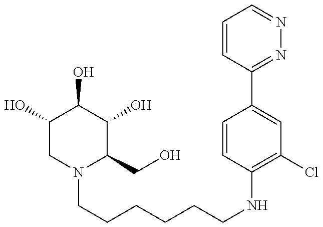 [SMILES: OC[C@@H]1[C@@H](O)[C@H](O)[C@@H](O)CN1CCCCCCNc1ccc(-c2cccnn2)cc1Cl] |
| 1203 | 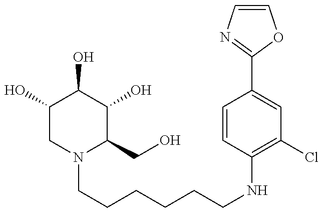 [SMILES: OC[C@@H]1[C@@H](O)[C@H](O)[C@@H](O)CN1CCCCCCNc1ccc(-c2ncco2)cc1Cl] |
| 1204 | 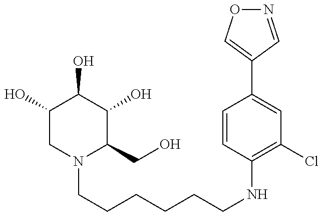 [SMILES: OC[C@@H]1[C@@H](O)[C@H](O)[C@@H](O)CN1CCCCCCNc1ccc(-c2cnoc2)cc1Cl] |
| 1205 | 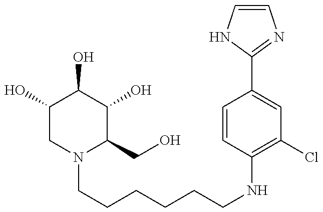 [SMILES: OC[C@@H]1[C@@H](O)[C@H](O)[C@@H](O)CN1CCCCCCNc1ccc(-c2ncc[nH]2)cc1Cl] |
| 1206 | 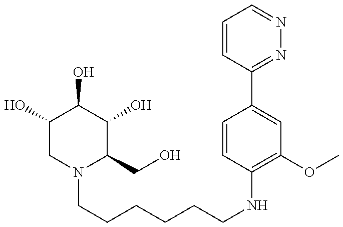 [SMILES: COc1cc(-c2cccnn2)ccc1NCCCCCCN1C[C@H](O)[C@@H](O)[C@H](O)[C@H]1CO] |
| 1207 | 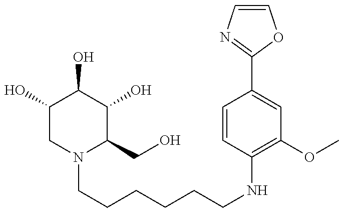 [SMILES: COc1cc(-c2ncco2)ccc1NCCCCCCN1C[C@H](O)[C@@H](O)[C@H](O)[C@H]1CO] |
| 1208 | 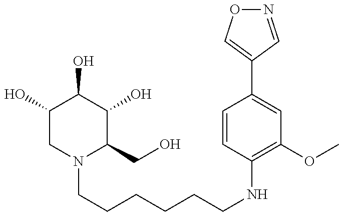 [SMILES: COc1cc(-c2cnoc2)ccc1NCCCCCCN1C[C@H](O)[C@@H](O)[C@H](O)[C@H]1CO] |
| 1209 | 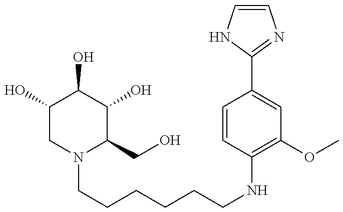 [SMILES: COc1cc(-c2ncc[nH]2)ccc1NCCCCCCN1C[C@H](O)[C@@H](O)[C@H](O)[C@H]1CO] |
| 1210 | 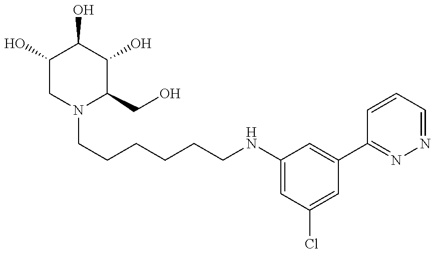 [SMILES: OC[C@@H]1[C@@H](O)[C@H](O)[C@@H](O)CN1CCCCCCNc1cc(Cl)cc(-c2cccnn2)c1] |
| 1211 | 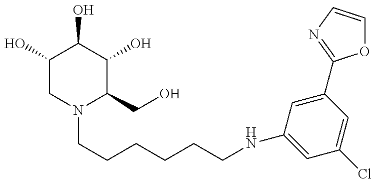 [SMILES: OC[C@@H]1[C@@H](O)[C@H](O)[C@@H](O)CN1CCCCCCNc1cc(Cl)cc(-c2ncco2)c1] |
| 1212 | 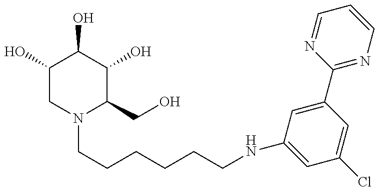 [SMILES: OC[C@@H]1[C@@H](O)[C@H](O)[C@@H](O)CN1CCCCCCNc1cc(Cl)cc(-c2ncccn2)c1] |
| 1213 | 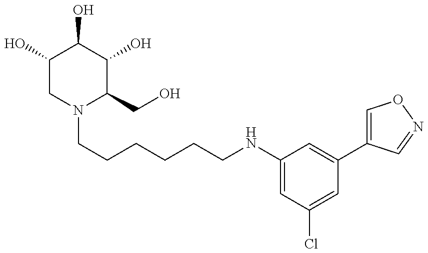 [SMILES: OC[C@@H]1[C@@H](O)[C@H](O)[C@@H](O)CN1CCCCCCNc1cc(Cl)cc(-c2cnoc2)c1] |
| 1214 | 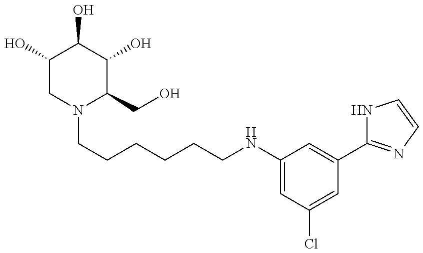 [SMILES: OC[C@@H]1[C@@H](O)[C@H](O)[C@@H](O)CN1CCCCCCNc1cc(Cl)cc(-c2ncc[nH]2)c1] |
| 1215 | 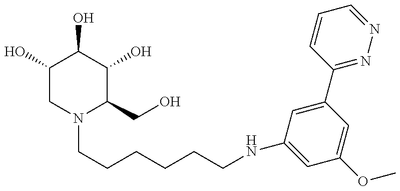 [SMILES: COc1cc(NCCCCCCN2C[C@H](O)[C@@H](O)[C@H](O)[C@H]2CO)cc(-c2cccnn2)c1] |
| 1216 | 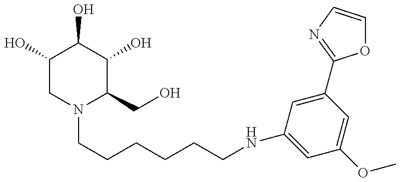 [SMILES: COc1cc(NCCCCCCN2C[C@H](O)[C@@H](O)[C@H](O)[C@H]2CO)cc(-c2ncco2)c1] |
| 1217 | 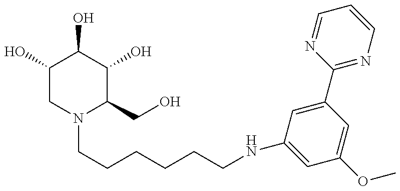 [SMILES: COc1cc(NCCCCCCN2C[C@H](O)[C@@H](O)[C@H](O)[C@H]2CO)cc(-c2ncccn2)c1] |
| 1218 | 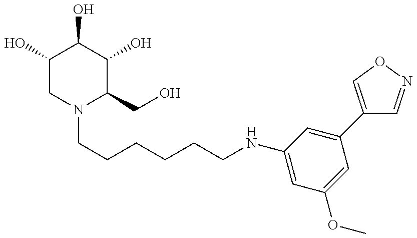 [SMILES: COc1cc(NCCCCCCN2C[C@H](O)[C@@H](O)[C@H](O)[C@H]2CO)cc(-c2cnoc2)c1] |
| 1219 | 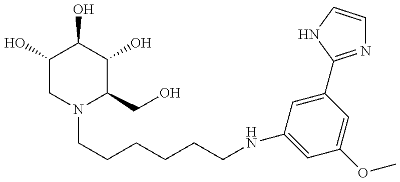 [SMILES: COc1cc(NCCCCCCN2C[C@H](O)[C@@H](O)[C@H](O)[C@H]2CO)cc(-c2ncc[nH]2)c1] |
| 1220 | 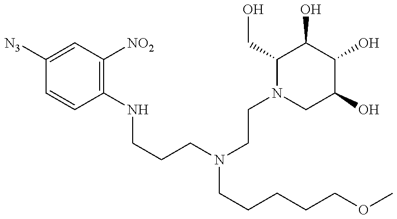 [SMILES: COCCCCCN(CCCNc1ccc(N=[N+]=[N-])cc1[N+](=O)[O-])CCN1C[C@H](O)[C@@H](O)[C@H](O)[C@H]1CO] |
| 1221 | 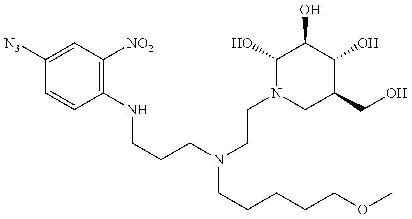 [SMILES: COCCCCCN(CCCNc1ccc(N=[N+]=[N-])cc1[N+](=O)[O-])CCN1C[C@H](CO)[C@@H](O)[C@H](O)[C@H]1O] |
| 1222 | 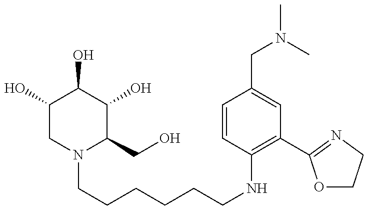 [SMILES: CN(C)Cc1ccc(NCCCCCCN2C[C@H](O)[C@@H](O)[C@H](O)[C@H]2CO)c(C2=NCCO2)c1] |
| 1223 | 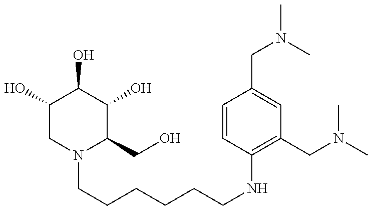 [SMILES: CN(C)Cc1ccc(NCCCCCCN2C[C@H](O)[C@@H](O)[C@H](O)[C@H]2CO)c(CN(C)C)c1] |
| 1224 | 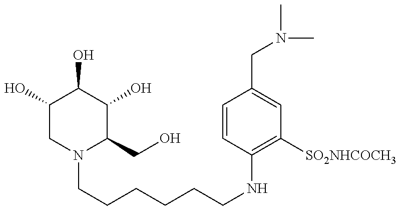 |
| 1225 | 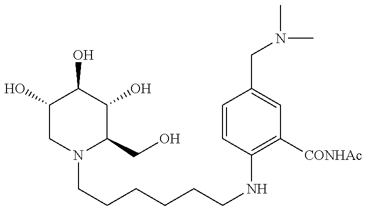 |
| 1226 | 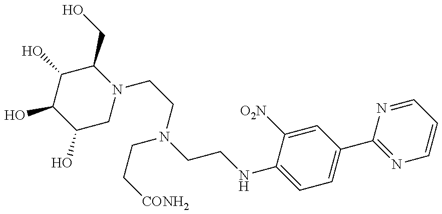 [SMILES: NC(=O)CCN(CCNc1ccc(-c2ncccn2)cc1[N+](=O)[O-])CCN1C[C@H](O)[C@@H](O)[C@H](O)[C@H]1CO] |
| 1227 | 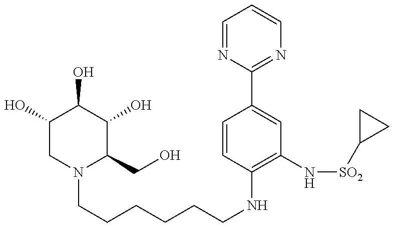 [SMILES: O=S(=O)(Nc1cc(-c2ncccn2)ccc1NCCCCCCN1C[C@H](O)[C@@H](O)[C@H](O)[C@H]1CO)C1CC1] |
| 1228 | 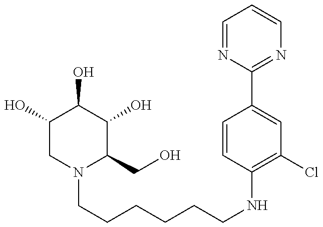 [SMILES: OC[C@@H]1[C@@H](O)[C@H](O)[C@@H](O)CN1CCCCCCNc1ccc(-c2ncccn2)cc1Cl] |
| 1229 | 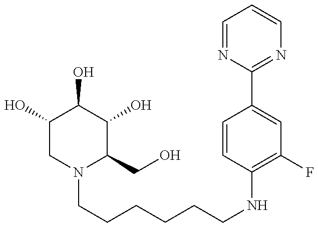 [SMILES: OC[C@@H]1[C@@H](O)[C@H](O)[C@@H](O)CN1CCCCCCNc1ccc(-c2ncccn2)cc1F] |
| 1230 | 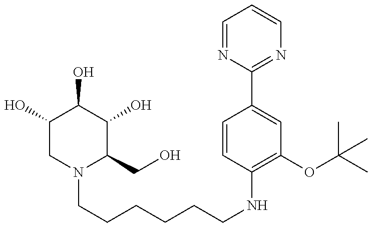 [SMILES: CC(C)(C)Oc1cc(-c2ncccn2)ccc1NCCCCCCN1C[C@H](O)[C@@H](O)[C@H](O)[C@H]1CO] |
| 1231 | 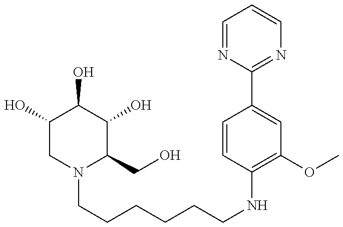 [SMILES: COc1cc(-c2ncccn2)ccc1NCCCCCCN1C[C@H](O)[C@@H](O)[C@H](O)[C@H]1CO] |
| 1232 | 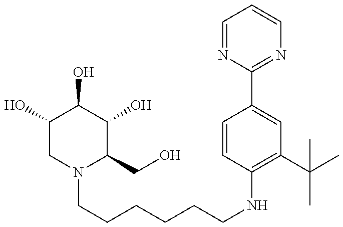 [SMILES: CC(C)(C)c1cc(-c2ncccn2)ccc1NCCCCCCN1C[C@H](O)[C@@H](O)[C@H](O)[C@H]1CO] |
| 1233 | 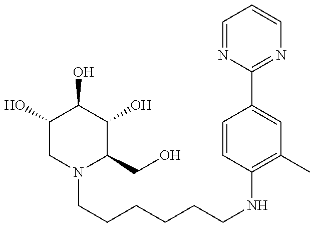 [SMILES: Cc1cc(-c2ncccn2)ccc1NCCCCCCN1C[C@H](O)[C@@H](O)[C@H](O)[C@H]1CO] |
| 1234 | 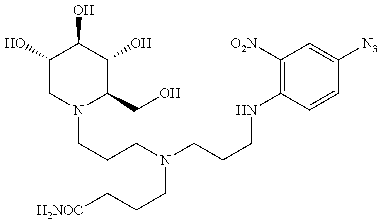 [SMILES: [N-]=[N+]=Nc1ccc(NCCCN(CCCC(N)=O)CCCN2C[C@H](O)[C@@H](O)[C@H](O)[C@H]2CO)c([N+](=O)[O-])c1] |
| 1235 | 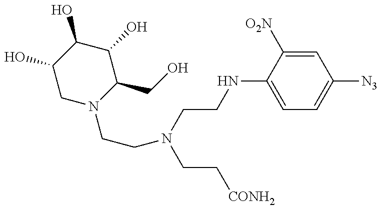 [SMILES: [N-]=[N+]=Nc1ccc(NCCN(CCC(N)=O)CCN2C[C@H](O)[C@@H](O)[C@H](O)[C@H]2CO)c([N+](=O)[O-])c1] |
| 1236 | 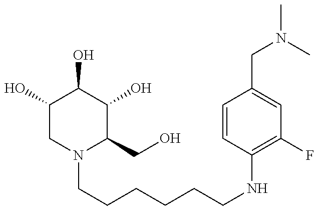 [SMILES: CN(C)Cc1ccc(NCCCCCCN2C[C@H](O)[C@@H](O)[C@H](O)[C@H]2CO)c(F)c1] |
| 1237 | 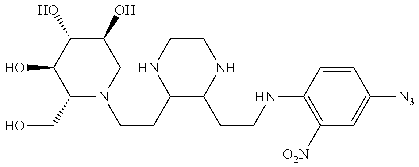 [SMILES: [N-]=[N+]=Nc1ccc(NCCC2NCCNC2CCN2C[C@H](O)[C@@H](O)[C@H](O)[C@H]2CO)c([N+](=O)[O-])c1] |
| 1238 | 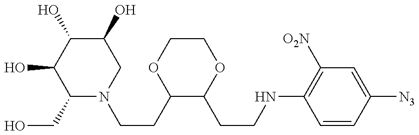 [SMILES: [N-]=[N+]=Nc1ccc(NCCC2OCCOC2CCN2C[C@H](O)[C@@H](O)[C@H](O)[C@H]2CO)c([N+](=O)[O-])c1] |
| 1239 | 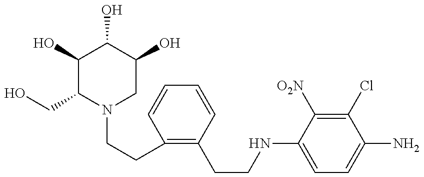 [SMILES: Nc1ccc(NCCc2ccccc2CCN2C[C@H](O)[C@@H](O)[C@H](O)[C@H]2CO)c([N+](=O)[O-])c1Cl] |
| 1240 | 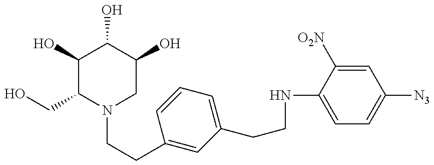 [SMILES: [N-]=[N+]=Nc1ccc(NCCc2cccc(CCN3C[C@H](O)[C@@H](O)[C@H](O)[C@H]3CO)c2)c([N+](=O)[O-])c1] |
| 1241 | 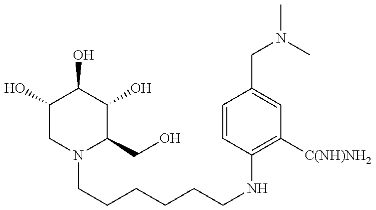 |
| 1242 | 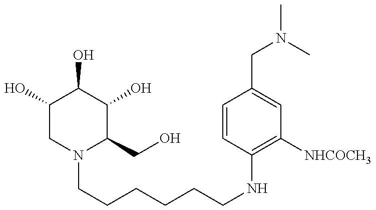 |
| 1243 | 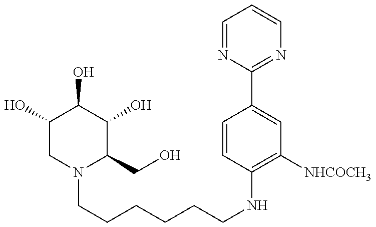 |
| 1244 | 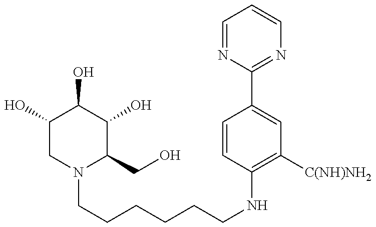 |
| 1245 | 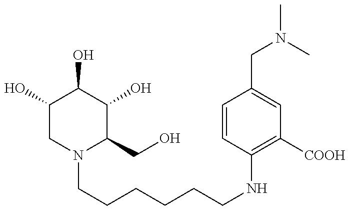 [SMILES: CN(C)Cc1ccc(NCCCCCCN2C[C@H](O)[C@@H](O)[C@H](O)[C@H]2CO)c(C(=O)O)c1] |
| 1246 | 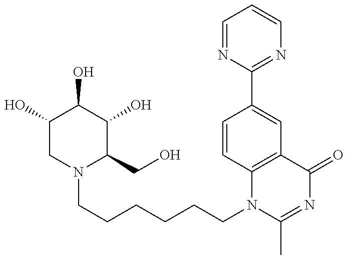 [SMILES: Cc1nc(=O)c2cc(-c3ncccn3)ccc2n1CCCCCCN1C[C@H](O)[C@@H](O)[C@H](O)[C@H]1CO] |
| 1247 | 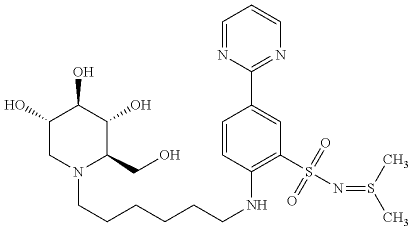 [SMILES: CS(C)=NS(=O)(=O)c1cc(-c2ncccn2)ccc1NCCCCCCN1C[C@H](O)[C@@H](O)[C@H](O)[C@H]1CO] |
| 1248 | 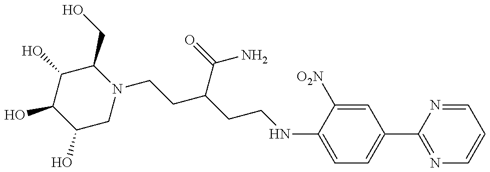 [SMILES: NC(=O)C(CCNc1ccc(-c2ncccn2)cc1[N+](=O)[O-])CCN1C[C@H](O)[C@@H](O)[C@H](O)[C@H]1CO] |
| 1249 | 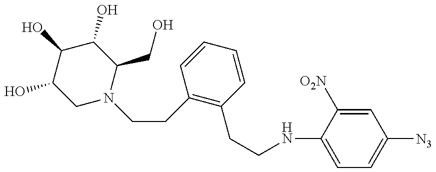 [SMILES: [N-]=[N+]=Nc1ccc(NCCc2ccccc2CCN2C[C@H](O)[C@@H](O)[C@H](O)[C@H]2CO)c([N+](=O)[O-])c1] |
| 1250 | 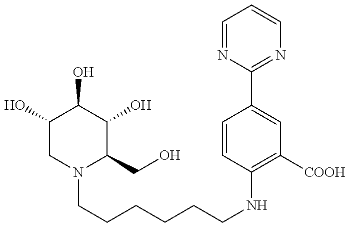 [SMILES: O=C(O)c1cc(-c2ncccn2)ccc1NCCCCCCN1C[C@H](O)[C@@H](O)[C@H](O)[C@H]1CO] |
| 1251 | 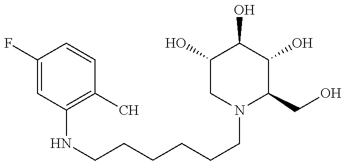 [SMILES: Cc1ccc(F)cc1NCCCCCCN1C[C@H](O)[C@@H](O)[C@H](O)[C@H]1CO] |
| 1252 | 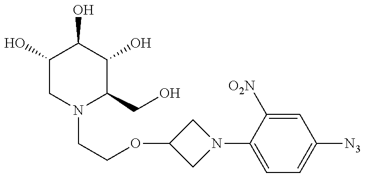 [SMILES: [N-]=[N+]=Nc1ccc(N2CC(OCCN3C[C@H](O)[C@@H](O)[C@H](O)[C@H]3CO)C2)c([N+](=O)[O-])c1] |
| 1253 | 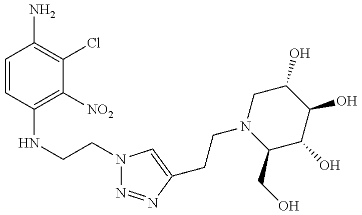 [SMILES: Nc1ccc(NCCn2cc(CCN3C[C@H](O)[C@@H](O)[C@H](O)[C@H]3CO)nn2)c([N+](=O)[O-])c1Cl] |
| 1254 | 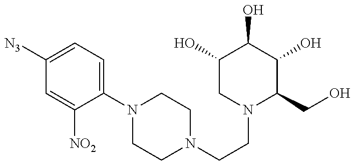 [SMILES: [N-]=[N+]=Nc1ccc(N2CCN(CCN3C[C@H](O)[C@@H](O)[C@H](O)[C@H]3CO)CC2)c([N+](=O)[O-])c1] |
| 1255 | 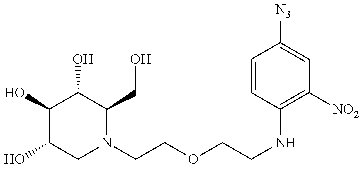 [SMILES: [N-]=[N+]=Nc1ccc(NCCOCCN2C[C@H](O)[C@@H](O)[C@H](O)[C@H]2CO)c([N+](=O)[O-])c1] |
| 1256 | 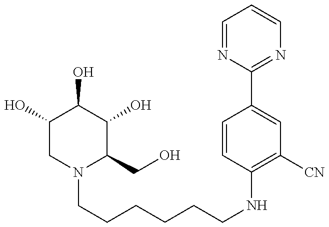 [SMILES: N#Cc1cc(-c2ncccn2)ccc1NCCCCCCN1C[C@H](O)[C@@H](O)[C@H](O)[C@H]1CO] |
| 1257 | 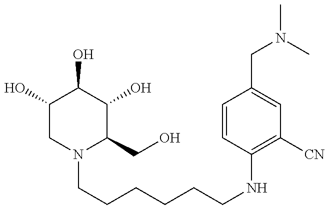 [SMILES: CN(C)Cc1ccc(NCCCCCCN2C[C@H](O)[C@@H](O)[C@H](O)[C@H]2CO)c(C#N)c1] |
| 1258 | 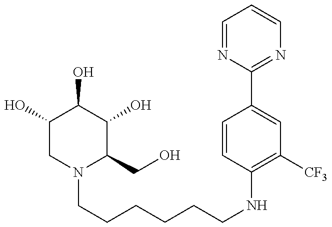 [SMILES: OC[C@@H]1[C@@H](O)[C@H](O)[C@@H](O)CN1CCCCCCNc1ccc(-c2ncccn2)cc1C(F)(F)F] |
| 1259 | 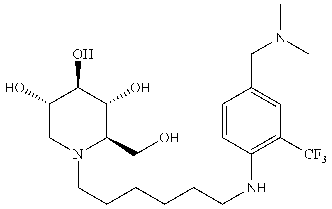 [SMILES: CN(C)Cc1ccc(NCCCCCCN2C[C@H](O)[C@@H](O)[C@H](O)[C@H]2CO)c(C(F)(F)F)c1] |
| 1260 | 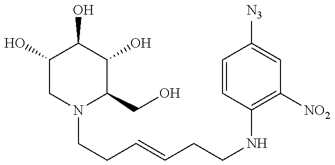 [SMILES: [N-]=[N+]=Nc1ccc(NCC/C=C/CCN2C[C@H](O)[C@@H](O)[C@H](O)[C@H]2CO)c([N+](=O)[O-])c1] |
| 1261 | 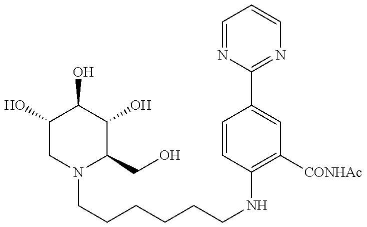 |
| 1262 | 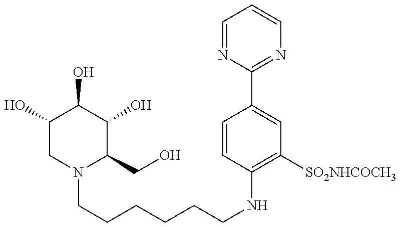 |
| 1263 | 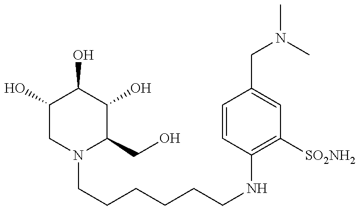 [SMILES: CN(C)Cc1ccc(NCCCCCCN2C[C@H](O)[C@@H](O)[C@H](O)[C@H]2CO)c(S(N)(=O)=O)c1] |
| 1264 | 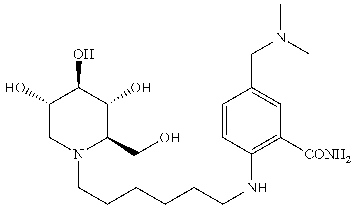 [SMILES: CN(C)Cc1ccc(NCCCCCCN2C[C@H](O)[C@@H](O)[C@H](O)[C@H]2CO)c(C(N)=O)c1] |
| 1265 | 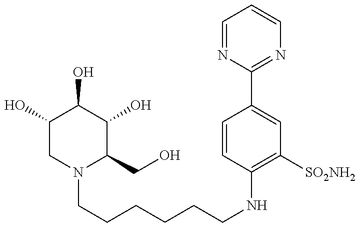 [SMILES: NS(=O)(=O)c1cc(-c2ncccn2)ccc1NCCCCCCN1C[C@H](O)[C@@H](O)[C@H](O)[C@H]1CO] |
| 1266 | 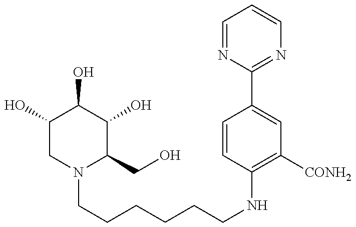 [SMILES: NC(=O)c1cc(-c2ncccn2)ccc1NCCCCCCN1C[C@H](O)[C@@H](O)[C@H](O)[C@H]1CO] |
| 1267 | 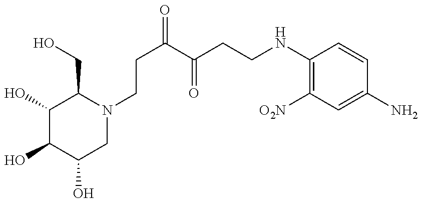 [SMILES: Nc1ccc(NCCC(=O)C(=O)CCN2C[C@H](O)[C@@H](O)[C@H](O)[C@H]2CO)c([N+](=O)[O-])c1] |
| 1268 | 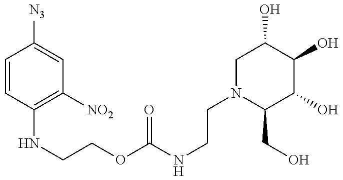 [SMILES: [N-]=[N+]=Nc1ccc(NCCOC(=O)NCCN2C[C@H](O)[C@@H](O)[C@H](O)[C@H]2CO)c([N+](=O)[O-])c1] |
| 1269 | 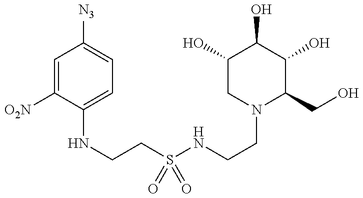 [SMILES: [N-]=[N+]=Nc1ccc(NCCS(=O)(=O)NCCN2C[C@H](O)[C@@H](O)[C@H](O)[C@H]2CO)c([N+](=O)[O-])c1] |
| 1270 | 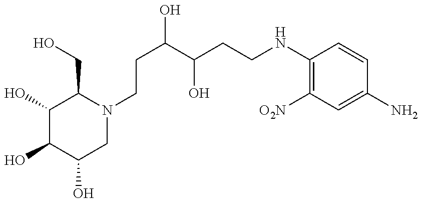 [SMILES: Nc1ccc(NCCC(O)C(O)CCN2C[C@H](O)[C@@H](O)[C@H](O)[C@H]2CO)c([N+](=O)[O-])c1] |
| 1271 | 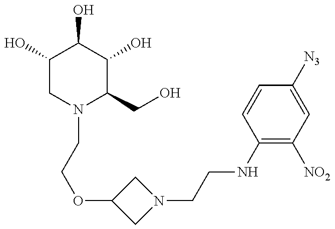 [SMILES: [N-]=[N+]=Nc1ccc(NCCN2CC(OCCN3C[C@H](O)[C@@H](O)[C@H](O)[C@H]3CO)C2)c([N+](=O)[O-])c1] |
| 1272 | 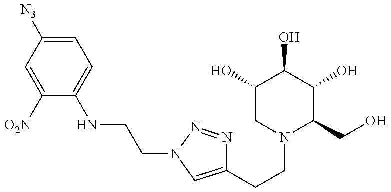 [SMILES: [N-]=[N+]=Nc1ccc(NCCn2cc(CCN3C[C@H](O)[C@@H](O)[C@H](O)[C@H]3CO)nn2)c([N+](=O)[O-])c1] |
| 1273 | 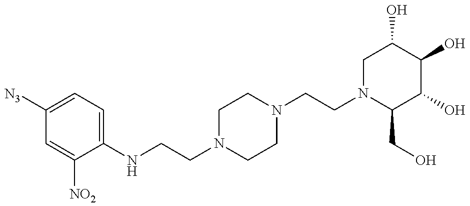 [SMILES: [N-]=[N+]=Nc1ccc(NCCN2CCN(CCN3C[C@H](O)[C@@H](O)[C@H](O)[C@H]3CO)CC2)c([N+](=O)[O-])c1] |
| 1274 | 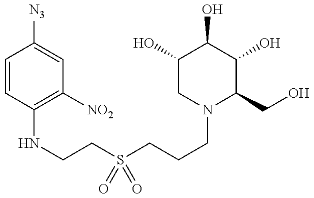 [SMILES: [N-]=[N+]=Nc1ccc(NCCS(=O)(=O)CCCN2C[C@H](O)[C@@H](O)[C@H](O)[C@H]2CO)c([N+](=O)[O-])c1] |
| 1275 | 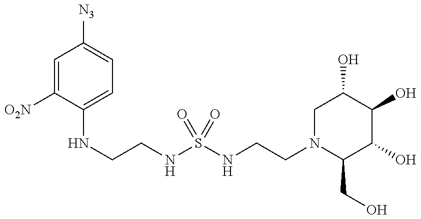 [SMILES: [N-]=[N+]=Nc1ccc(NCCNS(=O)(=O)NCCN2C[C@H](O)[C@@H](O)[C@H](O)[C@H]2CO)c([N+](=O)[O-])c1] |
| 1276 | 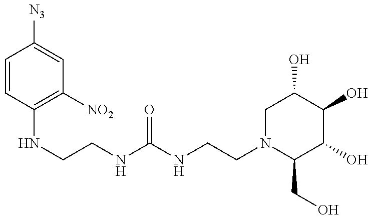 [SMILES: [N-]=[N+]=Nc1ccc(NCCNC(=O)NCCN2C[C@H](O)[C@@H](O)[C@H](O)[C@H]2CO)c([N+](=O)[O-])c1] |
| 1277 | 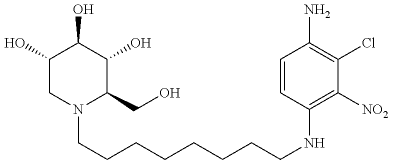 [SMILES: Nc1ccc(NCCCCCCCCN2C[C@H](O)[C@@H](O)[C@H](O)[C@H]2CO)c([N+](=O)[O-])c1Cl] |
| 1278 | 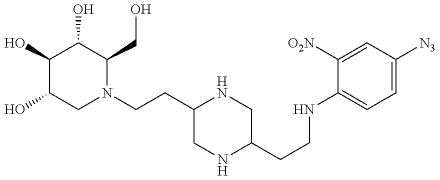 [SMILES: [N-]=[N+]=Nc1ccc(NCCC2CNC(CCN3C[C@H](O)[C@@H](O)[C@H](O)[C@H]3CO)CN2)c([N+](=O)[O-])c1] |
| 1279 | 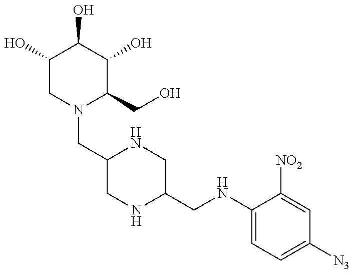 [SMILES: [N-]=[N+]=Nc1ccc(NCC2CNC(CN3C[C@H](O)[C@@H](O)[C@H](O)[C@H]3CO)CN2)c([N+](=O)[O-])c1] |
| 1280 | 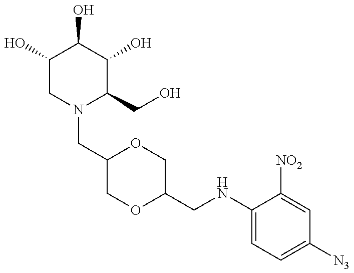 [SMILES: [N-]=[N+]=Nc1ccc(NCC2COC(CN3C[C@H](O)[C@@H](O)[C@H](O)[C@H]3CO)CO2)c([N+](=O)[O-])c1] |
| 1281 | 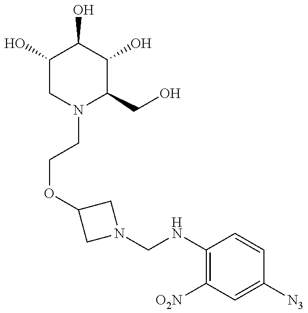 [SMILES: [N-]=[N+]=Nc1ccc(NCN2CC(OCCN3C[C@H](O)[C@@H](O)[C@H](O)[C@H]3CO)C2)c([N+](=O)[O-])c1] |
| 1282 | 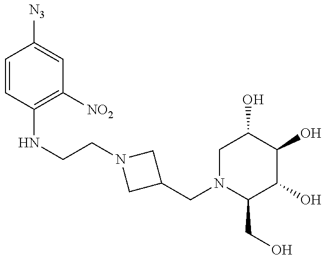 [SMILES: [N-]=[N+]=Nc1ccc(NCCN2CC(CN3C[C@H](O)[C@@H](O)[C@H](O)[C@H]3CO)C2)c([N+](=O)[O-])c1] |
| 1283 | 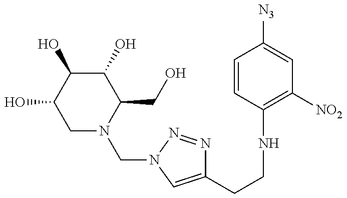 [SMILES: [N-]=[N+]=Nc1ccc(NCCc2cn(CN3C[C@H](O)[C@@H](O)[C@H](O)[C@H]3CO)nn2)c([N+](=O)[O-])c1] |
| 1284 | 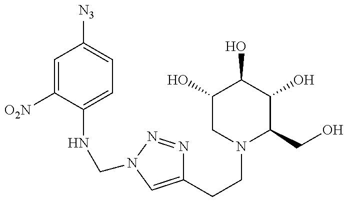 [SMILES: [N-]=[N+]=Nc1ccc(NCn2cc(CCN3C[C@H](O)[C@@H](O)[C@H](O)[C@H]3CO)nn2)c([N+](=O)[O-])c1] |
| 1285 | 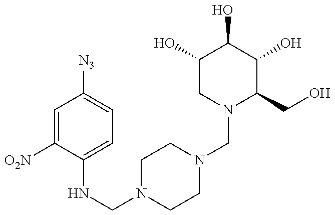 [SMILES: [N-]=[N+]=Nc1ccc(NCN2CCN(CN3C[C@H](O)[C@@H](O)[C@H](O)[C@H]3CO)CC2)c([N+](=O)[O-])c1] |
| 1286 | 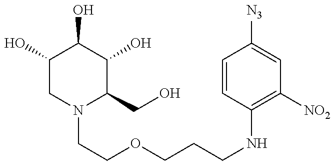 [SMILES: [N-]=[N+]=Nc1ccc(NCCCOCCN2C[C@H](O)[C@@H](O)[C@H](O)[C@H]2CO)c([N+](=O)[O-])c1] |
| 1287 | 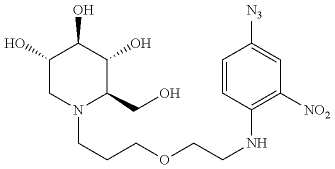 [SMILES: [N-]=[N+]=Nc1ccc(NCCOCCCN2C[C@H](O)[C@@H](O)[C@H](O)[C@H]2CO)c([N+](=O)[O-])c1] |
| 1288 | 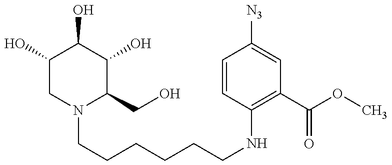 [SMILES: COC(=O)c1cc(N=[N+]=[N-])ccc1NCCCCCCN1C[C@H](O)[C@@H](O)[C@H](O)[C@H]1CO] |
| 1289 | 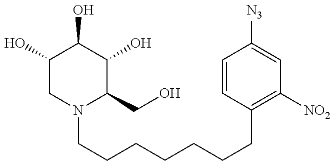 [SMILES: [N-]=[N+]=Nc1ccc(CCCCCCCN2C[C@H](O)[C@@H](O)[C@H](O)[C@H]2CO)c([N+](=O)[O-])c1] |
| 1290 | 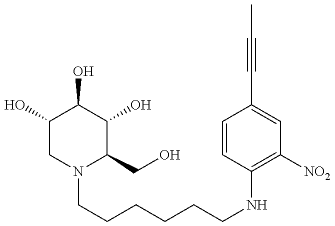 [SMILES: CC#Cc1ccc(NCCCCCCN2C[C@H](O)[C@@H](O)[C@H](O)[C@H]2CO)c([N+](=O)[O-])c1] |
| 1291 | 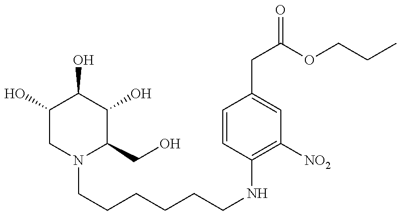 [SMILES: CCCOC(=O)Cc1ccc(NCCCCCCN2C[C@H](O)[C@@H](O)[C@H](O)[C@H]2CO)c([N+](=O)[O-])c1] |
| 1292 | 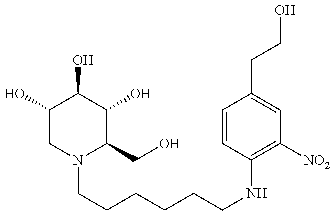 [SMILES: O=[N+]([O-])c1cc(CCO)ccc1NCCCCCCN1C[C@H](O)[C@@H](O)[C@H](O)[C@H]1CO] |
| 1293 | 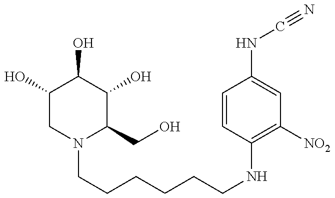 [SMILES: N#CNc1ccc(NCCCCCCN2C[C@H](O)[C@@H](O)[C@H](O)[C@H]2CO)c([N+](=O)[O-])c1] |
| 1294 | 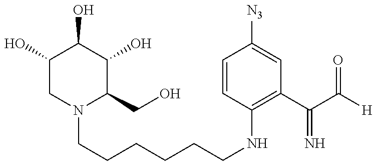 [SMILES: [N-]=[N+]=Nc1ccc(NCCCCCCN2C[C@H](O)[C@@H](O)[C@H](O)[C@H]2CO)c(C(=N)C=O)c1] |
| 1295 | 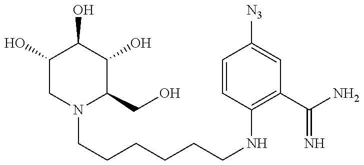 [SMILES: [N-]=[N+]=Nc1ccc(NCCCCCCN2C[C@H](O)[C@@H](O)[C@H](O)[C@H]2CO)c(C(=N)N)c1] |
| 1296 | 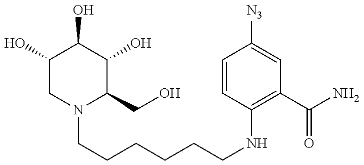 [SMILES: [N-]=[N+]=Nc1ccc(NCCCCCCN2C[C@H](O)[C@@H](O)[C@H](O)[C@H]2CO)c(C(N)=O)c1] |
| 1297 | 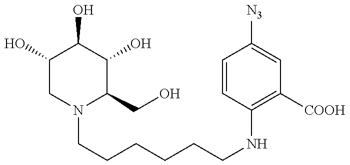 [SMILES: [N-]=[N+]=Nc1ccc(NCCCCCCN2C[C@H](O)[C@@H](O)[C@H](O)[C@H]2CO)c(C(=O)O)c1] |
| 1298 | 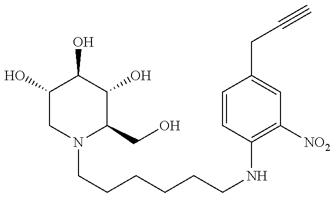 [SMILES: C#CCc1ccc(NCCCCCCN2C[C@H](O)[C@@H](O)[C@H](O)[C@H]2CO)c([N+](=O)[O-])c1] |
| 1299 | 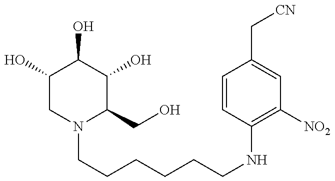 [SMILES: N#CCc1ccc(NCCCCCCN2C[C@H](O)[C@@H](O)[C@H](O)[C@H]2CO)c([N+](=O)[O-])c1] |
| 1300 | 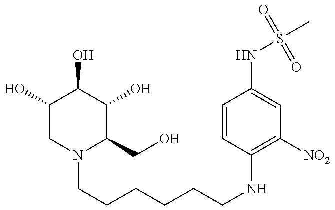 [SMILES: CS(=O)(=O)Nc1ccc(NCCCCCCN2C[C@H](O)[C@@H](O)[C@H](O)[C@H]2CO)c([N+](=O)[O-])c1] |
| 1301 | 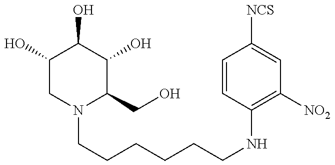 |
| 1302 | 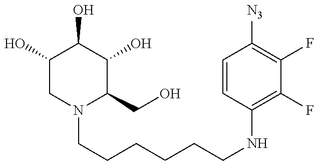 [SMILES: [N-]=[N+]=Nc1ccc(NCCCCCCN2C[C@H](O)[C@@H](O)[C@H](O)[C@H]2CO)c(F)c1F] |
| 1303 | 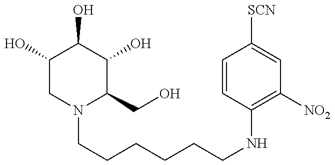 |
| 1304 | 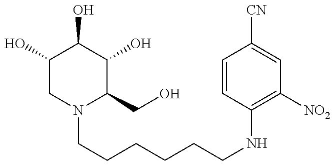 [SMILES: N#Cc1ccc(NCCCCCCN2C[C@H](O)[C@@H](O)[C@H](O)[C@H]2CO)c([N+](=O)[O-])c1] |
| 1305 | 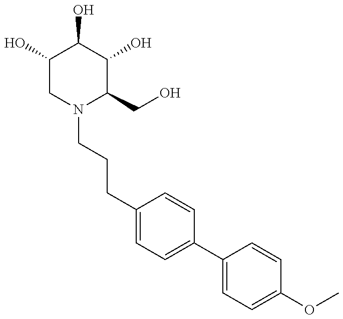 [SMILES: COc1ccc(-c2ccc(CCCN3C[C@H](O)[C@@H](O)[C@H](O)[C@H]3CO)cc2)cc1] |
| 1306 | 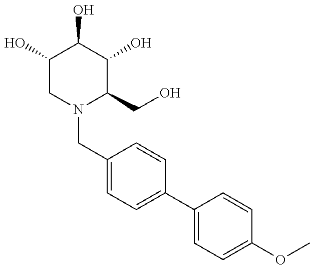 [SMILES: COc1ccc(-c2ccc(CN3C[C@H](O)[C@@H](O)[C@H](O)[C@H]3CO)cc2)cc1] |
| 1307 | 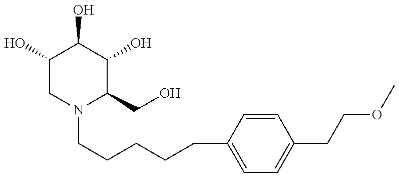 [SMILES: COCCc1ccc(CCCCCN2C[C@H](O)[C@@H](O)[C@H](O)[C@H]2CO)cc1] |
| 1308 | 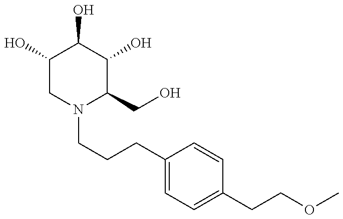 [SMILES: COCCc1ccc(CCCN2C[C@H](O)[C@@H](O)[C@H](O)[C@H]2CO)cc1] |
| 1309 | 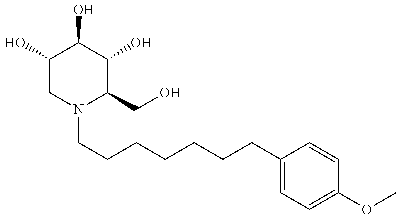 [SMILES: COc1ccc(CCCCCCCN2C[C@H](O)[C@@H](O)[C@H](O)[C@H]2CO)cc1] |
| 1310 | 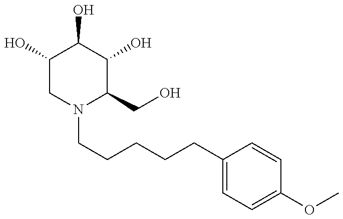 [SMILES: COc1ccc(CCCCCN2C[C@H](O)[C@@H](O)[C@H](O)[C@H]2CO)cc1] |
| 1311 | 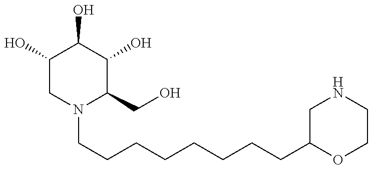 [SMILES: OC[C@@H]1[C@@H](O)[C@H](O)[C@@H](O)CN1CCCCCCCCC1CNCCO1] |
| 1312 | 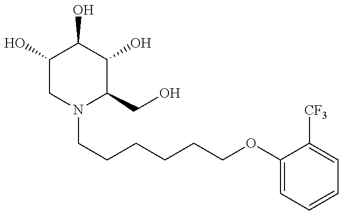 [SMILES: OC[C@@H]1[C@@H](O)[C@H](O)[C@@H](O)CN1CCCCCCOc1ccccc1C(F)(F)F] |
| 1313 | 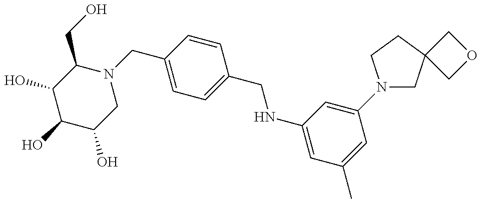 [SMILES: Cc1cc(NCc2ccc(CN3C[C@H](O)[C@@H](O)[C@H](O)[C@H]3CO)cc2)cc(N2CCC3(COC3)C2)c1] |
| 1314 | 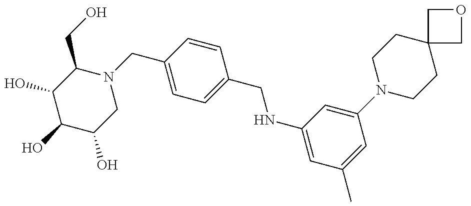 [SMILES: Cc1cc(NCc2ccc(CN3C[C@H](O)[C@@H](O)[C@H](O)[C@H]3CO)cc2)cc(N2CCC3(CC2)COC3)c1] |
| 1315 | 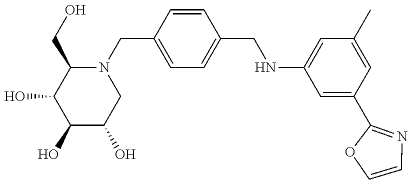 [SMILES: Cc1cc(NCc2ccc(CN3C[C@H](O)[C@@H](O)[C@H](O)[C@H]3CO)cc2)cc(-c2ncco2)c1] |
| 1316 | 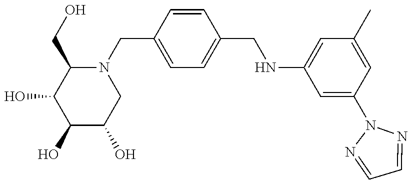 [SMILES: Cc1cc(NCc2ccc(CN3C[C@H](O)[C@@H](O)[C@H](O)[C@H]3CO)cc2)cc(-n2nccn2)c1] |
| 1317 | 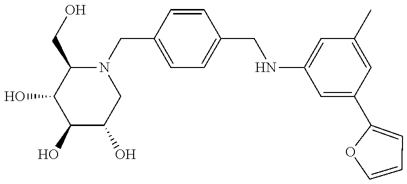 [SMILES: Cc1cc(NCc2ccc(CN3C[C@H](O)[C@@H](O)[C@H](O)[C@H]3CO)cc2)cc(-c2ccco2)c1] |
| 1318 | 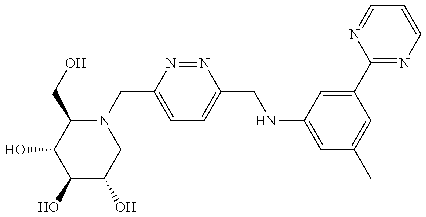 [SMILES: Cc1cc(NCc2ccc(CN3C[C@H](O)[C@@H](O)[C@H](O)[C@H]3CO)nn2)cc(-c2ncccn2)c1] |
| 1319 | 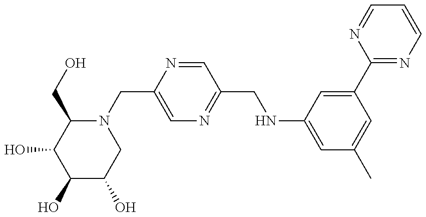 [SMILES: Cc1cc(NCc2cnc(CN3C[C@H](O)[C@@H](O)[C@H](O)[C@H]3CO)cn2)cc(-c2ncccn2)c1] |
| 1320 | 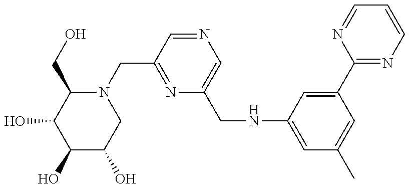 [SMILES: Cc1cc(NCc2cncc(CN3C[C@H](O)[C@@H](O)[C@H](O)[C@H]3CO)n2)cc(-c2ncccn2)c1] |
| 1321 | 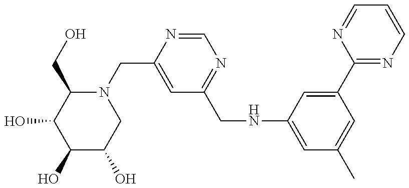 [SMILES: Cc1cc(NCc2cc(CN3C[C@H](O)[C@@H](O)[C@H](O)[C@H]3CO)ncn2)cc(-c2ncccn2)c1] |
| 1322 | 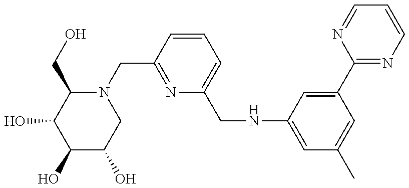 [SMILES: Cc1cc(NCc2cccc(CN3C[C@H](O)[C@@H](O)[C@H](O)[C@H]3CO)n2)cc(-c2ncccn2)c1] |
| 1323 | 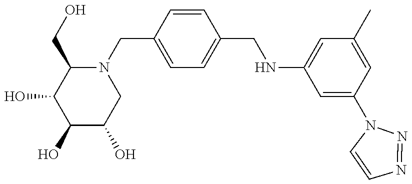 [SMILES: Cc1cc(NCc2ccc(CN3C[C@H](O)[C@@H](O)[C@H](O)[C@H]3CO)cc2)cc(-n2ccnn2)c1] |
| 1324 | 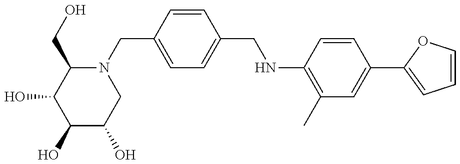 [SMILES: Cc1cc(-c2ccco2)ccc1NCc1ccc(CN2C[C@H](O)[C@@H](O)[C@H](O)[C@H]2CO)cc1] |
| 1325 | 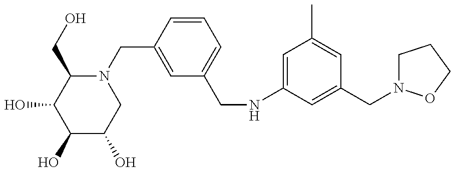 [SMILES: Cc1cc(CN2CCCO2)cc(NCc2cccc(CN3C[C@H](O)[C@@H](O)[C@H](O)[C@H]3CO)c2)c1] |
| 1326 | 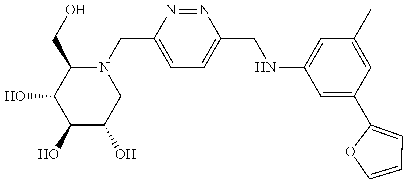 [SMILES: Cc1cc(NCc2ccc(CN3C[C@H](O)[C@@H](O)[C@H](O)[C@H]3CO)nn2)cc(-c2ccco2)c1] |
| 1327 | 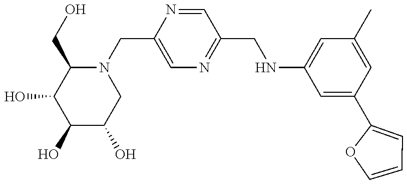 [SMILES: Cc1cc(NCc2cnc(CN3C[C@H](O)[C@@H](O)[C@H](O)[C@H]3CO)cn2)cc(-c2ccco2)c1] |
| 1328 | 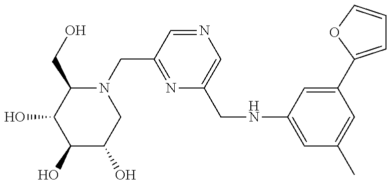 [SMILES: Cc1cc(NCc2cncc(CN3C[C@H](O)[C@@H](O)[C@H](O)[C@H]3CO)n2)cc(-c2ccco2)c1] |
| 1329 | 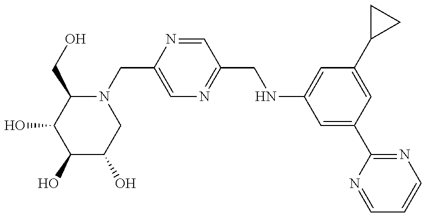 [SMILES: OC[C@@H]1[C@@H](O)[C@H](O)[C@@H](O)CN1Cc1cnc(CNc2cc(-c3ncccn3)cc(C3CC3)c2)cn1] |
| 1330 | 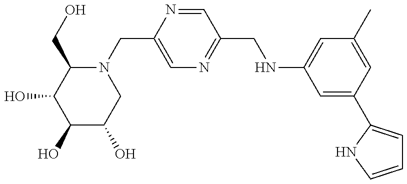 [SMILES: Cc1cc(NCc2cnc(CN3C[C@H](O)[C@@H](O)[C@H](O)[C@H]3CO)cn2)cc(-c2ccc[nH]2)c1] |
| 1331 | 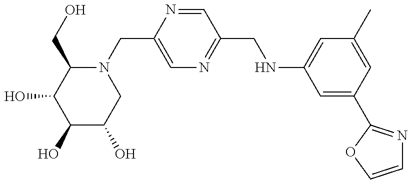 [SMILES: Cc1cc(NCc2cnc(CN3C[C@H](O)[C@@H](O)[C@H](O)[C@H]3CO)cn2)cc(-c2ncco2)c1] |
| 1332 | 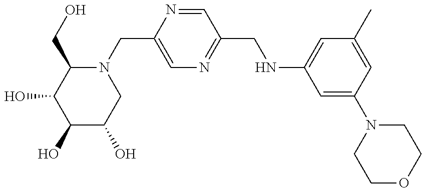 [SMILES: Cc1cc(NCc2cnc(CN3C[C@H](O)[C@@H](O)[C@H](O)[C@H]3CO)cn2)cc(N2CCOCC2)c1] |
| 1333 | 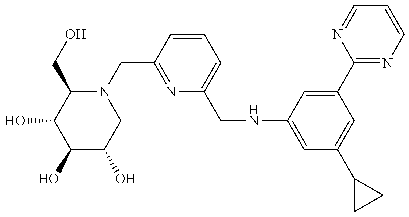 [SMILES: OC[C@@H]1[C@@H](O)[C@H](O)[C@@H](O)CN1Cc1cccc(CNc2cc(-c3ncccn3)cc(C3CC3)c2)n1] |
| 1334 | 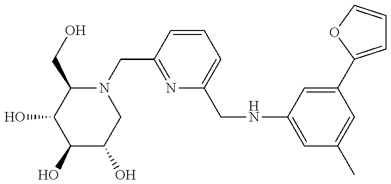 [SMILES: Cc1cc(NCc2cccc(CN3C[C@H](O)[C@@H](O)[C@H](O)[C@H]3CO)n2)cc(-c2ccco2)c1] |
| 1335 | 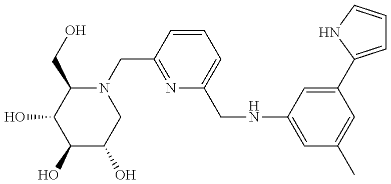 [SMILES: Cc1cc(NCc2cccc(CN3C[C@H](O)[C@@H](O)[C@H](O)[C@H]3CO)n2)cc(-c2ccc[nH]2)c1] |
| 1336 | 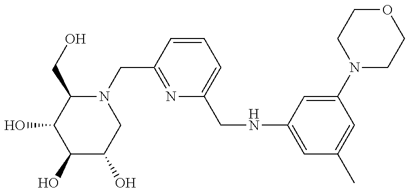 [SMILES: Cc1cc(NCc2cccc(CN3C[C@H](O)[C@@H](O)[C@H](O)[C@H]3CO)n2)cc(N2CCOCC2)c1] |
| 1337 | 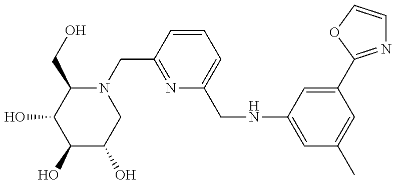 [SMILES: Cc1cc(NCc2cccc(CN3C[C@H](O)[C@@H](O)[C@H](O)[C@H]3CO)n2)cc(-c2ncco2)c1] |
| 1338 | 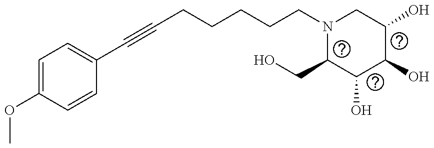 [SMILES: COc1ccc(C#CCCCCCN2C[C@H](O)[C@@H](O)[C@H](O)[C@H]2CO)cc1] |
| 1339 | 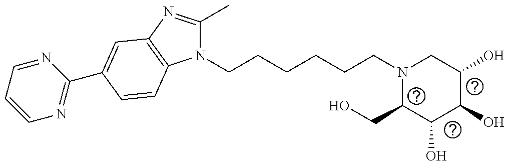 [SMILES: Cc1nc2cc(-c3ncccn3)ccc2n1CCCCCCN1C[C@H](O)[C@@H](O)[C@H](O)[C@H]1CO] |

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NCH₃, R⁶

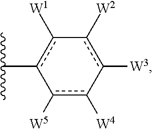

[symbol]

are single bonds, W¹, W³, and W⁵ are hydrogen, W² is cyclopropyl, and W⁴ is pyridazine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

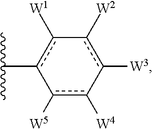

[symbol]

are single bonds, W¹, W³, and W⁵ are hydrogen, W² is methanesulfonyl, and W⁴ is morpholine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

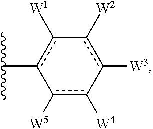

[symbol]

are single bonds, W¹, W³, and W⁵ are hydrogen, W² is methyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

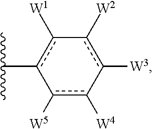

[symbol]

are single bonds, W¹, W³, and W⁵ are hydrogen, W² is methanesulfonyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

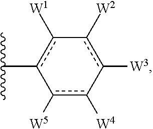

[symbol]

are single bonds, W¹, W³, and W⁵ are hydrogen, W² is methyl, and W⁴ is morpholine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

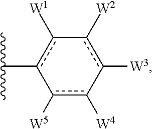

[symbol]

are single bonds, W¹, W³, and W⁵ are hydrogen, W² is cyclopropyl, and W⁴ is morpholine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

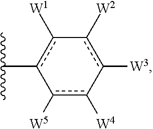

[symbol]

are single bonds, W¹, W³, and W⁵ are hydrogen, W² is cyclopropyl, and W⁴ is pyrrole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

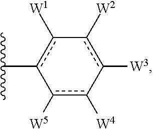

[symbol]

are single bonds, W¹, W³, and W⁵ are hydrogen, W² is cyclopropyl, and W⁴ is pyridazine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

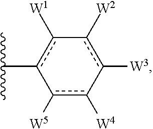

[symbol]

are single bonds, W¹, W³, and W⁵ are hydrogen, W² is cyclopropyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

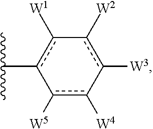

[symbol]

are single bonds, W¹, W³, and W⁵ are hydrogen, W² is cyclopropyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

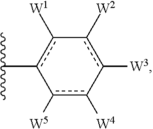

[symbol]

are single bonds, W¹, W³, and W⁵ are hydrogen, W² is cyclopropyl, and W⁴ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

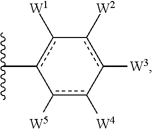

[symbol]

are single bonds, W¹, W³, and W⁵ are hydrogen, W² is cyclopropyl, and W⁴ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

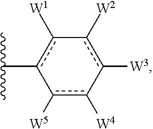

[symbol]

are single bonds, W¹, W³, and W⁵ are hydrogen, W² is cyclopropyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

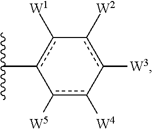

[symbol]

are single bonds, W¹ is nitro, W², W⁴, and W⁵ are hydrogen, and W³ is 2,6-dimethylmorpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

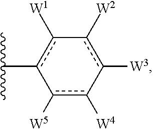

[symbol]

are single bonds, W¹ is nitro, W², W⁴, and W⁵ are hydrogen, and W³ is 2-oxa-5-azabicyclo[2.2.1]heptan-5-yl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

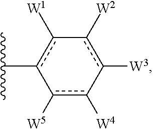

[symbol]

are single bonds, W¹ is nitro, W², W⁴, and W⁵ are hydrogen, and W³ is 2-oxa-6-azaspiro[3.4]octan-6-yl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

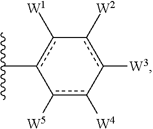

[symbol]

are single bonds, W¹ is nitro, W², W⁴, and W⁵ are hydrogen, and W³ is (1R,5S)-3-oxa-8-azabicyclo[3.2.1]octan-8-yl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

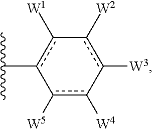

[symbol]

are single bonds, W¹ is nitro, W², W⁴, and W⁵ are hydrogen, and W³ is 2-oxa-7-azaspiro[3.5]nonan-7-yl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NCH₃ is

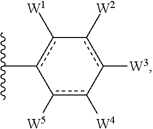

[symbol]

are single bonds, W¹ is nitro, W², W⁴, and W⁵ are hydrogen, and W³ is methyl(2-nitro-4-[(1,2-oxazolidin-2-yl)]methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

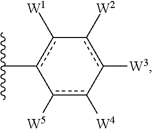

[symbol]

are single bonds, W² is bromo, W¹, W³, and W⁵ are hydrogen, and W⁴ is methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

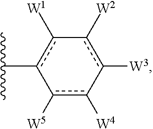

[symbol]

are single bonds, W² is bromo, W¹, W³, and W⁵ are hydrogen, and W⁴ is methanesulfonyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

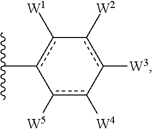

[symbol]

are single bonds, W² is bromo, W¹, W³, and W⁵ are hydrogen, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

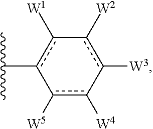

[symbol]

are single bonds, W² is bromo, W¹, W³, and W⁵ are hydrogen, and W⁴ is methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

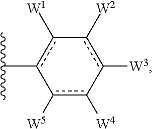

[symbol]

are single bonds, W² is bromo, W¹, W³, and W⁵ are hydrogen, and W⁴ is methanesulfonyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

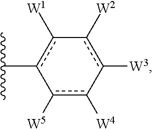

[symbol]

are single bonds, W² is bromo, W¹, W³, and W⁵ are hydrogen, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

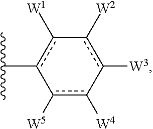

[symbol]

are single bonds, W¹, W², W⁴, and W⁵ are hydrogen, and W³ is bromo.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

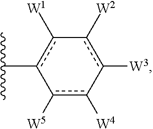

[symbol]

are single bonds, W¹, W², and W⁴ are hydrogen, W⁵ is nitro, and W³ is bromo.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

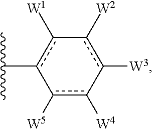

[symbol]

are single bonds, W¹, W³, and W⁵ are hydrogen, W² is methyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

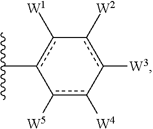

[symbol]

are single bonds, W¹, W³, and W⁵ are hydrogen, W² is methyl, and W⁴ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

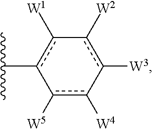

[symbol]

are single bonds, W¹, W³, and W⁵ are hydrogen, W² is methyl, and W⁴ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

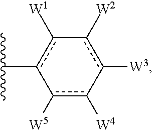

[symbol]

are single bonds, W¹, W³, and W⁵ are hydrogen, W² is methyl, and W⁴ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

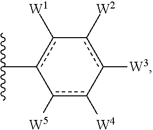

[symbol]

are single bonds, W¹, W³, and W⁵ are hydrogen, W² is methyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

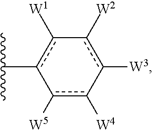

[symbol]

are single bonds, W¹, W³, and W⁵ are hydrogen, W² is methanesulfonyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

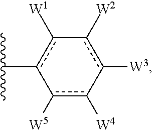

[symbol]

are single bonds, W¹, W³, and W⁵ are hydrogen, W² is methanesulfonyl, and W⁴ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

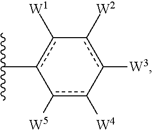

[symbol]

are single bonds, W¹, W³, and W⁵ are hydrogen, W² is methanesulfonyl, and W⁴ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

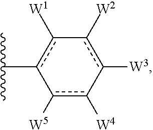

[symbol]

are single bonds, W¹, W³, and W⁵ are hydrogen, W² is methanesulfonyl, and W⁴ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

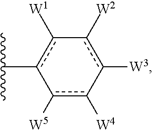

[symbol]

are single bonds, W¹, W³, and W⁵ are hydrogen, W² is methanesulfonyl, and W⁴ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

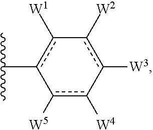

[symbol]

are single bonds, W¹, W³, and W⁵ are hydrogen, W² is methanesulfonyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

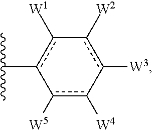

[symbol]

are single bonds, W¹, W³, and W⁵ are hydrogen, W² is cyano, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

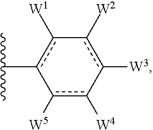

[symbol]

are single bonds, W¹, W³, and W⁵ are hydrogen, W² is cyano, and W⁴ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

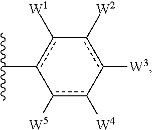

[symbol]

are single bonds, W¹, W³, and W⁵ are hydrogen, W² is cyano, and W⁴ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

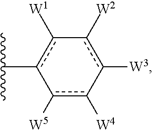

[symbol]

are single bonds, W¹, W³, and W⁵ are hydrogen, W² is cyano, and W⁴ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

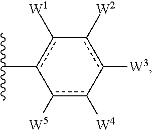

[symbol]

are single bonds, W¹, W³, and W⁵ are hydrogen, W² is cyano, and W⁴ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

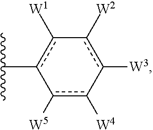

[symbol]

are single bonds, W¹, W³, and W⁵ are hydrogen, W² is methanesulfonyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

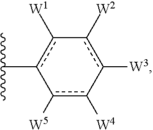

[symbol]

are single bonds, W¹, W³, and W⁵ are hydrogen, W² is methyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

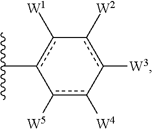

[symbol]

are single bonds, W¹, W³, and W⁵ are hydrogen, W² is methyl, and W⁴ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

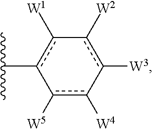

[symbol]

are single bonds, W¹, W³, and W⁵ are hydrogen, W² is methyl, and W⁴ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

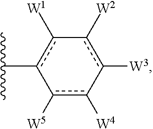

[symbol]

are single bonds, W¹, W³, and W⁵ are hydrogen, W is methyl, and W is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

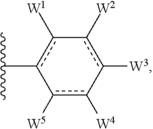

[symbol]

are single bonds, W¹, W³, and W⁵ are hydrogen, W² is methyl, and W⁴ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

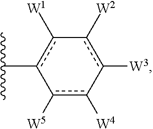

[symbol]

are single bonds, W¹, W³, and W⁵ are hydrogen, W² is methyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

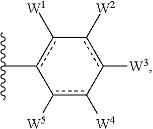

[symbol]

are single bonds, W¹, W³, and W⁵ are hydrogen, W² is methanesulfonyl, and W⁴ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

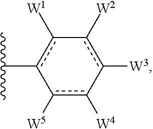

[symbol]

are single bonds, W¹, W³, and W⁵ are hydrogen, W² is methanesulfonyl, and W⁴ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

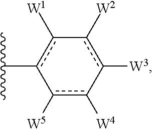

[symbol]

are single bonds, W¹, W³, and W⁵ are hydrogen, W² is methanesulfonyl, and W⁴ is 2,3-dihydro-1,4-dioxine.

In another aspect, a compound of the disclosure comprises Formula (I), wherein A¹, A², A³ and A⁴ are hydroxyl, R¹ is CH₂, R² is absent, R³ is phenyl, R⁴ is CH₂, R⁵ is NH, R⁶ is

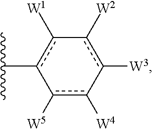

[symbol]

are single bonds, W¹, W³, and W⁵ are hydrogen, W² is methanesulfonyl, and W⁴ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

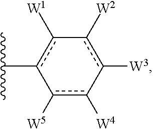

[symbol]

are single bonds, W¹, W³, and W⁵ are hydrogen, W² is methanesulfonyl, and W⁴ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

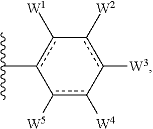

[symbol]

are single bonds, W¹, W³, and W⁵ are hydrogen, W² is methanesulfonyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

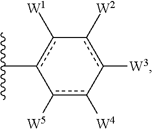

[symbol]

are single bonds, W¹, W³, and W⁵ are hydrogen, W² is morpholine, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

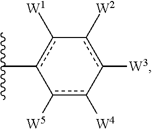

[symbol]

are single bonds, W¹, W³, and W⁵ are hydrogen, W² is (1,2-oxazolidin-2-yl)methyl, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

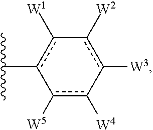

[symbol]

are single bonds, W¹, W³, and W⁵ are hydrogen, W² is 2,3-dihydro-1,4-dioxine, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

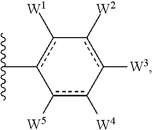

[symbol]

are single bonds, W¹, W³, and W⁵ are hydrogen, W² is pyrrole, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

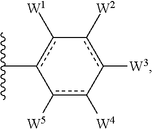

[symbol]

are single bonds, W¹, W³, and W⁵ are hydrogen, W² is pyridazine, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

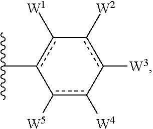

[symbol]

are single bonds, W¹, W³, and W⁵ are hydrogen, W² is pyrimidine, and W⁴ is cyano.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

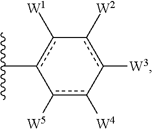

[symbol]

are single bonds, W¹, W², W⁴, and W⁵ are hydrogen, and W³ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

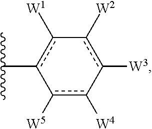

[symbol]

are single bonds, W², W⁴, and W⁵ are hydrogen, W¹ is nitro, and W³ is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

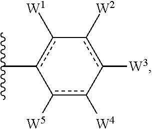

[symbol]

are single bonds, W¹, W², W⁴, and W⁵ are hydrogen, and W³ is morpholine.

In another aspect, a compound of the disclosure comprises Formula (I), wherein A¹, A², A³, and A⁴ are hydroxyl, R¹ is (CH₂)₂, R² is absent, R³ is indole, R⁴ is (CH₂)₂, R⁵ is NH, R⁶ is

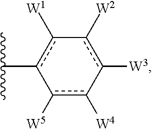

[symbol]

are single bonds, W¹, W³, and W⁵ are hydrogen, W² is pyridazine, and W⁴ is methoxy.

In another aspect, a compound of the disclosure comprises Formula (I), wherein A¹, A², A³, and A⁴ are hydroxyl, R¹ is (CH₂)₂, R² is absent, R³ is indole, R⁴ is (CH₂)₂, R⁵ is NH, R⁶ is

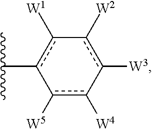

[symbol]

are single bonds, W¹, W³, and W⁵ are hydrogen, W² is furan, and W⁴ is chloro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

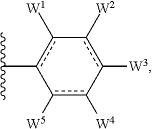

[symbol]

are single bonds, W¹, W³, and W⁵ are hydrogen, W² is 2,3-dihydrofuran, and W⁴ is methoxy.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

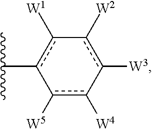

[symbol]

are single bonds, W¹, W³, and W⁵ are hydrogen, W² is methoxy, and W⁴ is 1,2,4-oxadiazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

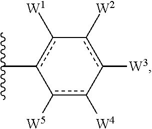

[symbol]

are single bonds, W¹, W³, and W⁵ are hydrogen, W² is pyrimidine, and W⁴ is cyclopropoxy.

In another aspect, a compound of the disclosure comprises Formula (I), wherein A¹, A², A³, and A⁴ are hydroxyl, R¹ is (CH₂)₂, R² is absent, R³ is indole, R⁴ is (CH₂)₂, R⁵ is NH, R⁶ is

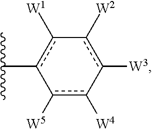

[symbol]

are single bonds, W¹, W³, and W⁵ are hydrogen, W² is bromo, and W⁴ is methoxy.

In another aspect, a compound of the disclosure comprises Formula (I), wherein A¹, A², A³, and A⁴ are hydroxyl, R¹ is (CH₂)₂, R² is absent, R³ is indole, R⁴ is (CH₂)₂, R⁵ is NH, R⁶ is

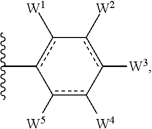

[symbol]

are single bonds, and W¹, W², W³, W⁴, and W⁵ are hydrogen.

In another aspect, a compound of the disclosure comprises Formula (I), wherein A¹, A², A³, and A⁴ are hydroxyl, R¹ is (CH₂)₂, R² is absent, R³ is isopropylbenzene, R⁴ is (CH₂)₂, R⁵ is NH, R⁶ is

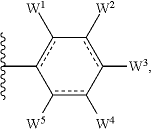

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

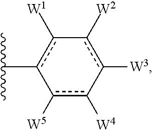

[symbol]

are single bonds, W², W⁴, and W⁵ are hydrogen, W¹ is nitro, and W³ is imidazole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is

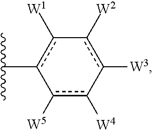

[symbol]

are single bonds, W¹, W², W⁴, and W⁵ are hydrogen, and W³ is 5-propyl-1,2,4-oxadiazole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

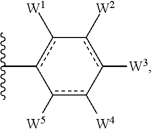

[symbol]

are single bonds, W¹, W², W⁴, and W⁵ are hydrogen, and W³ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

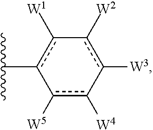

[symbol]

are single bonds, W¹, W², W⁴, and W⁵ are hydrogen, and W is morpholine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

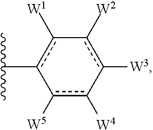

[symbol]

are single bonds, W¹, W², W⁴, and W⁵ are hydrogen, and W³ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

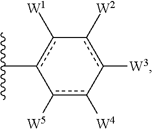

[symbol]

are single bonds, W¹, W², W⁴, and W⁵ are hydrogen, and W³ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

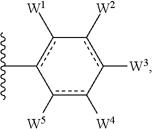

[symbol]

are single bonds, W¹, W², W⁴, and W⁵ are hydrogen, and W³ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

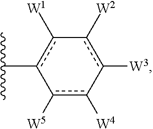

[symbol]

are single bonds, W¹, W², and W⁴ are hydrogen, W⁵ is nitro, and W³ is 5-propyl-1,2,4-oxadiozaole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

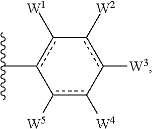

[symbol]

are single bonds, W¹, W², and W⁴ are hydrogen, W⁵ is nitro, and W³ is 5-propyl-1,2,4-(1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

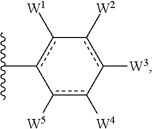

[symbol]

are single bonds, W¹, W², and W⁴ are hydrogen, W⁵ is nitro, and W³ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

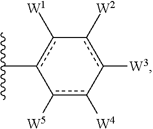

[symbol]

are single bonds, W¹, W², and W⁴ are hydrogen, W⁵ is nitro, and W³ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

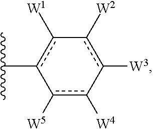

[symbol]

are single bonds, W¹, W², and W⁴ are hydrogen, W⁵ is nitro, and W³ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

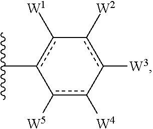

[symbol]

are single bonds, W¹, W², and W⁴ are hydrogen, W⁵ is nitro, and W³ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

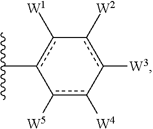

[symbol]

are single bonds, W¹, W², and W⁴ are hydrogen, W⁵ is nitro, and W³ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

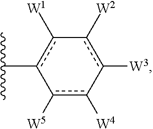

[symbol]

are single bonds, W¹, W², W⁴, and W⁵ are hydrogen, and W³ is 5-propyl-1,2,4-(1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

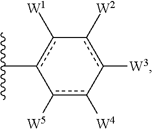

[symbol]

are single bonds, W¹, W², W⁴, and W⁵ are hydrogen, and W³ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

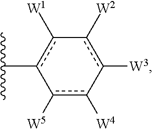

[symbol]

are single bonds, W¹, W², W⁴, and W⁵ are hydrogen, and W³ is 2,3-dihydro-1,4,-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

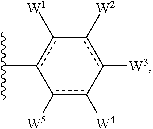

[symbol]

are single bonds, W¹, W², W⁴, and W⁵ are hydrogen, and W³ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

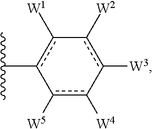

[symbol]

are single bonds, W¹, W², W⁴, and W⁵ are hydrogen, and W³ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

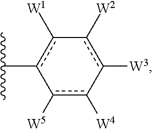

[symbol]

are single bonds, W¹, W², W⁴, and W⁵ are hydrogen, and W³ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

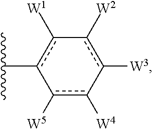

[symbol]

are single bonds, W¹, W², and W⁴ are hydrogen, W⁵ is nitro, and W³ is 5-propyl-1,2,4-oxadiazole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

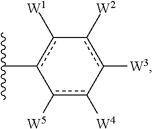

[symbol]

are single bonds, W¹, W², and W⁴ are hydrogen, W⁵ is nitro, and W³ is (1,2-oxazolidin-2-yl)methyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

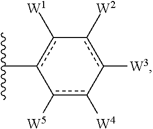

[symbol]

are single bonds, W¹, W², and W⁴ are hydrogen, W⁵ is nitro, and W³ is 2,3-dihydro-1,4-dioxine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

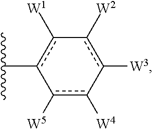

[symbol]

are single bonds, W¹, W², and W⁴ are hydrogen, W⁵ is nitro, and W³ is pyrrole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

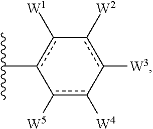

[symbol]

are single bonds, W¹, W², and W⁴ are hydrogen, W⁵ is nitro, and W³ is pyridazine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

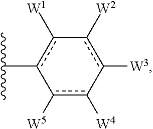

[symbol]

are single bonds, W¹, W², and W⁴ are hydrogen, W⁵ is nitro, and W³ is pyrimidine.

In another aspect, a compound of the disclosure comprises Formula (I), wherein A¹, A², A³, and A⁴ are hydroxyl, R¹ is (CH₂)₂, R² is absent, R³ is bicyclo[1.1.1]pentan-1-yl)methyl, R⁴ is (CH₂)₂, R⁵ is NH, R⁶ is

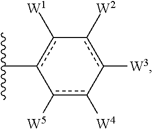

[symbol]

are single bonds, W¹, W², and W⁴ are hydrogen, W⁵ is nitro, and W³ is azide.

In another aspect, a compound of the disclosure comprises Formula (I), wherein A¹, A², A³, and A⁴ are hydroxyl, R¹ is CH₂, R² is absent, R³ is bicyclo[1.1.1]pentan-1-yl)methyl, R⁴ is CH₂, R⁵ is N, R⁶ is

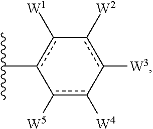

[symbol]

are single bonds, W¹, W², and W⁴ are hydrogen, W⁵ is nitro, and W³ is azide.

In another aspect, a compound of the disclosure comprises Formula (I), wherein A¹, A², A³, and A⁴ are hydroxyl, R¹ is CH₂, R² is absent, R³ is diphenylmethane, R⁴ is CH₂, R⁵ is NH, R⁶ is

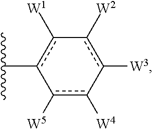

[symbol]

are single bonds, W¹, W², and W⁴ are hydrogen, W⁵ is nitro, and W³ is azide.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

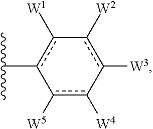

[symbol]

are single bonds, W¹, W², and W⁴ are hydrogen, W⁵ is nitro, and W³ is azide.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

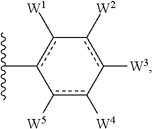

[symbol]

are single bonds, W¹, W², and W⁴ are hydrogen, W⁵ is nitro, and W³ is (1,2-oxazolidin-2-yl)methyl.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

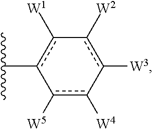

[symbol]

are single bonds, W¹, W², and W⁴ are hydrogen, W⁵ is nitro, and W³ is pyridazine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

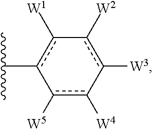

[symbol]

are single bonds, W¹, W², and W⁴ are hydrogen, W⁵ is nitro, and W³ is pyridine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

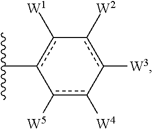

[symbol]

are single bonds, W¹, W², and W⁴ are hydrogen, W⁵ is nitro, and W³ is pyrimidine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

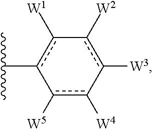

[symbol]

are single bonds, W¹, W², and W⁴ are hydrogen, W⁵ is nitro, and W³ is oxazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

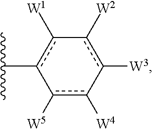

[symbol]

are single bonds, W¹, W³, and W⁵ are hydrogen, W² is chloro, and W⁴ is oxadiazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

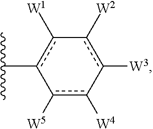

[symbol]

are single bonds, W¹, W³, and W⁵ are hydrogen, W² is pyrimidine, and W⁴ is cyclopropanesulfonamide.

In another aspect, a compound of the disclosure comprises Formula (I), wherein A¹, A², A³, and A⁴ are hydroxyl, R¹ is (CH₂)₂, R² is absent, R³ is cyclohexylphenyl, R⁴ is (CH₂)₂, R⁵ is

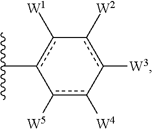

[symbol]

are single bonds, W¹, W², and W⁴ are hydrogen, W⁵ is nitro, and W³ is azide.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

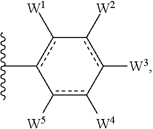

[symbol]

are single bonds, W¹, W³, and W⁵ are hydrogen, W² is chloro, and W⁴ is COOCH₃.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

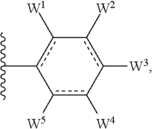

[symbol]

are single bonds, W¹, W³, and W⁵ are hydrogen, W² is chloro, and W⁴ is COOH.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

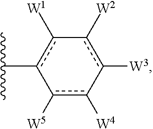

[symbol]

are single bonds, W¹, W³, and W⁵ are hydrogen, W² is pyrimidine, and W⁴ is COOH.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

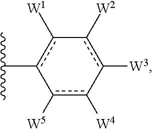

[symbol]

are single bonds, W¹, W³, and W⁵ are hydrogen, W² is chloro, and W⁴ is 5-phenyl-1,2,4-oxadiazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

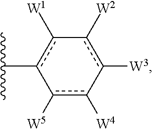

[symbol]

are single bonds, W¹, W³, and W⁵ are hydrogen, W² is methoxy, and W⁴ is 5-cyclobutyl-1,2,4-oxadiazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

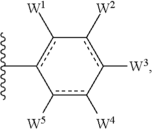

[symbol]

are single bonds, W¹, W³, and W⁵ are hydrogen, W² is methoxy, and W⁴ is 5-butyl-1,2,4-oxadiazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

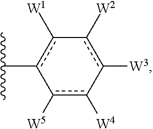

[symbol]

are single bonds, W¹, W³, and W⁵ are hydrogen, W² is chloro, and W⁴ is 5-cyclobutyl-1,2,4-oxadiazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

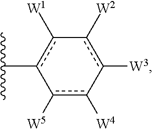

[symbol]

are single bonds, W¹, W³, and W⁵ are hydrogen, W² is chloro, and W⁴ is 5-propyl-1,2,4-oxadiazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

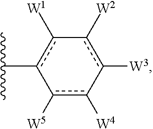

[symbol]

are single bonds, W¹, W³, and W⁵ are hydrogen, W² is pyrimidine, and W⁴ is 1,1-dimethoxyethane.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

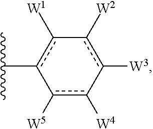

[symbol]

are single bonds, W¹, W³, and W⁵ are hydrogen, W² is pyrimidine, and W⁴ is acetamide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

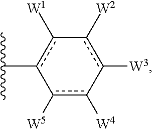

[symbol]

are single bonds, W¹, W³, and W⁵ are hydrogen, W² is pyrimidine, and W⁴ is N,N-dimethylacetamide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

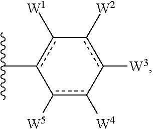

[symbol]

are single bonds, W¹, W³, and W⁵ are hydrogen, W² is pyrimidine, and W⁴ is tert-butoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

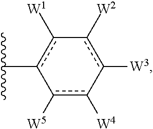

[symbol]

are single bonds, W¹, W³, and W⁵ are hydrogen, W² is pyrimidine, and W⁴ is methanesulfonyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

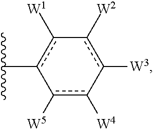

[symbol]

are single bonds, W¹, W³, and W⁵ are hydrogen, W² is pyrimidine, and W⁴ is hydroxymethyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

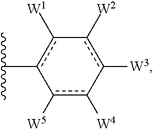

[symbol]

are single bonds, W¹, W³, and W⁵ are hydrogen, W² is pyrimidine, and W⁴ is cyano.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

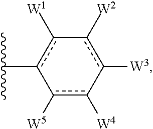

[symbol]

are single bonds, W¹, W³, and W⁵ are hydrogen, W² is pyrimidine, and W⁴ is trifluoromethyl.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

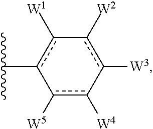

[symbol]

are single bonds, W¹, W³, and W⁵ are hydrogen, W² is methoxy, and W⁴ is 5-phenyl-1,2,4-oxadiazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

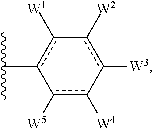

[symbol]

are single bonds, W¹, W³, and W⁵ are hydrogen, W² is methoxy, and W⁴ is cyano.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

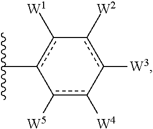

[symbol]

are single bonds, W¹, W³, and W⁵ are hydrogen, W² is chloro, and W⁴ is cyano.

In another aspect, a compound of the disclosure comprises Formula (I), wherein A¹, A², A³, and A⁴ are hydroxyl, R¹ is (CH₂)₂, R² is absent, R³ is chlorophenyl, R⁴ is NH(CH₂)₂, R⁵ is NH, R⁶ is

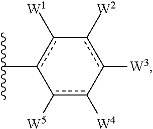

[symbol]

are single bonds, W², W⁴ and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

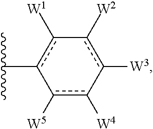

[symbol]

are single bonds, W¹, W², and W⁴ are hydrogen, W³ is cyclopropyl, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵

### is NH, R⁶ is

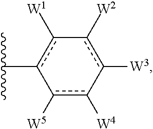

[symbol]

are single bonds, W¹, W², and W⁴ are hydrogen, W³ is cyclohexyl, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

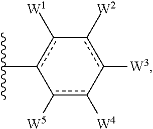

[symbol]

are single bonds, W¹, W², and W⁴ are hydrogen, W³ is phenyl, and W⁵ is nitro.

In another aspect, a compound of the disclosure comprises Formula (I), wherein A¹, A², A³, and A⁴ are hydroxyl, R¹ is (CH₂)₂, R² is absent, R³ is tert-butylphenyl, R⁴ is (CH₂)₂, R⁵ is NH, R⁶ is

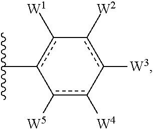

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is bromophenyl, R⁴ is (CH₂)₂, R⁵ is NH, R⁶ is

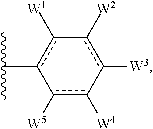

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

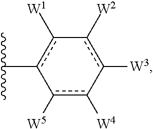

[symbol]

are single bonds, W¹, W³, and W⁵ are hydrogen, W² is methyltetrahydrofuran, and W⁴ is methoxy.

In another aspect, a compound of the disclosure comprises Formula (I), wherein A¹, A², A³, and A⁴ are hydroxyl, R¹ is (CH₂)₂, R² is absent, R³ is tert-butylphenyl, R⁴ is NH(CH₂)₂, R⁵ is NH, R⁶ is

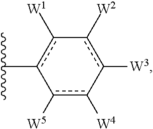

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is chlorophenyl, R⁴ is NH(CH₂)₂, R⁵ is NH, R⁶ is

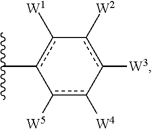

[symbol]

are single bonds, W¹, W² and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is methoxyphenyl, R⁴ is NH(CH₂)₂, R⁵ is NH, R⁶ is

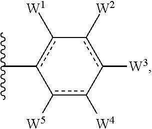

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

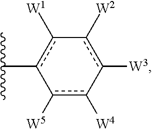

[symbol]

are single bonds, W¹, W³, and W⁵ are hydrogen, W² is benzyl, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

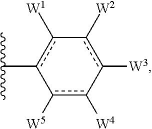

[symbol]

are single bonds, W¹, W³, and W⁵ are hydrogen, W² is tetrahydrofuran, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

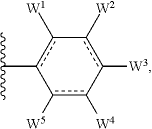

[symbol]

are single bonds, W¹, W³, and W⁵ are hydrogen, W² is 2,3-dihydrofuran, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

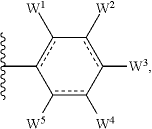

[symbol]

are single bonds, W¹, W³, and W⁵ are hydrogen, W² is cyclohexane, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

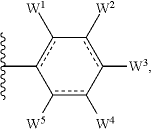

[symbol]

are single bonds, W¹, W³, and W⁵ are hydrogen, W² is cyclohexene, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

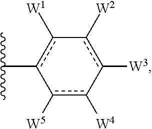

[symbol]

are single bonds, W¹, W³, and W⁵ are hydrogen, W² is phenyl, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

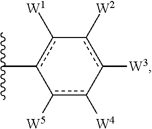

[symbol]

are single bonds, W¹, W³, and W⁵ are hydrogen, W² is pyrimidine, and W⁴ is tertbutyl.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

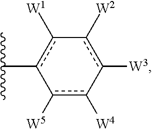

[symbol]

are single bonds, W¹, W³, and W⁵ are hydrogen, W² is methoxy, and W⁴ is imidazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

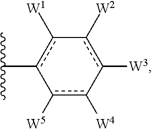

[symbol]

are single bonds, W¹, W³, and W⁵ are hydrogen, W² is methoxy, and W⁴ is furan.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

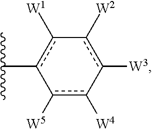

[symbol]

are single bonds, W¹, W³, and W⁵ are hydrogen, W² is methoxy, and W⁴ is 3,6-dihydro-2H-pyran.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

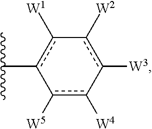

[symbol]

are single bonds, W¹, W³, and W⁵ are hydrogen, W² is methoxy, and W⁴ is 2,3-dihydro-1,4-dioxine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

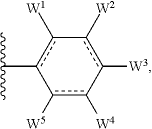

[symbol]

are single bonds, W¹, W³, and W⁵ are hydrogen, W² is methoxy, and W⁴ is pyrimidine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

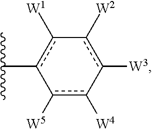

[symbol]

are single bonds, W¹, W³, and W⁵ are hydrogen, W² is methoxy, and W⁴ is pyridazine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

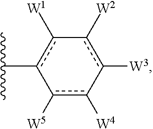

[symbol]

are single bonds, W¹, W³, and W⁵ are hydrogen, W² is chloro, and W⁴ is imidazole.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

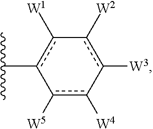

[symbol]

are single bonds, W¹, W³, and W⁵ are hydrogen, W² is chloro, and W⁴ is furan.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

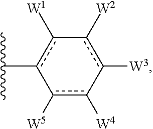

[symbol]

are single bonds, W¹, W³, and W⁵ are hydrogen, W² is chloro, and W⁴ is 3,6-dihydro-2H-pyran.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

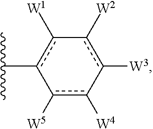

[symbol]

are single bonds, W¹, W³, and W⁵ are hydrogen, W² is chloro, and W⁴ is 2,3-dihydro-1,4-dioxine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

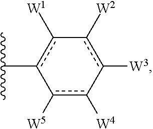

[symbol]

are single bonds, W¹, W³, and W⁵ are hydrogen, W² is chloro, and W⁴ is pyrimidine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

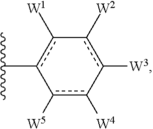

[symbol]

are single bonds, W¹, W³, and W⁵ are hydrogen, W² is chloro, and W⁴ is pyridazine.

In another aspect, a compound of the disclosure comprises Formula (I), wherein A¹, A², A³, and A⁴ are hydroxyl, R¹ is CH₂, R² is absent, R³ is thiophene, R⁴ is (CH₂)₂, R⁵ is NH, R⁶ is

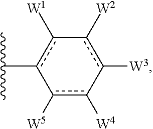

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is biphenyl, R⁴ is absent, R⁵ is NH, R⁶ is

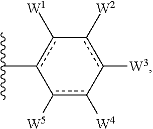

[symbol]

are single bonds, W¹, W², and W⁴ are hydrogen, W³ is azide, and W⁵ is nitro.

In another aspect, a compound of the disclosure comprises Formula (I), wherein A¹, A², A³ and A⁴ are hydroxyl, R² is CH₂, R² is absent, R³ is biphenyl, R⁴ is CH₂, R⁵ is NH, R⁶ is

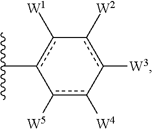

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is CH₂, R² is absent, R³ is biphenyl, R⁴ is absent, R⁵ is NH, R⁶ is

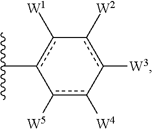

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is absent, R² is absent, R³ is biphenyl, R⁴ is absent, R⁵ is NH, R⁶ is

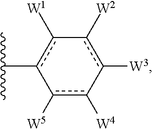

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

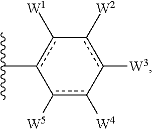

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is cyclohexylphenyl, R⁴ is (CH₂)₂, R⁵ is NH, R⁶ is

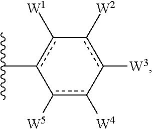

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is 2,3-dihydro-1H-indene, R⁴ is (CH₂)₂, R⁵ is NH, R⁶ is

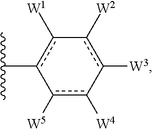

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is 2,3-dihydrobenzo[b]1,4]dioxine, R⁴ is (CH₂)₂, R⁵ is NH, R⁶ is

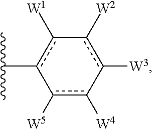

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is thiophene, R⁴ is CH₂, R⁵ is NH, R⁶ is

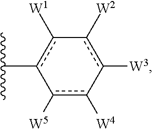

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is NH, R³ is benzthiazole, R⁴ is (CH₂)₂, R⁵ is NH, R⁶ is

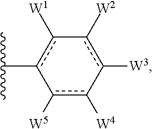

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is NH, R³ is 1,2,3,4-tetrahydronaphthalene, R⁴ is (CH₂)₂, R⁵ is NH, R⁶ is

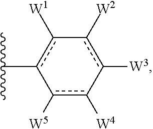

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is indole, R⁴ is (CH₂)₂, R⁵ is NH, R⁶ is

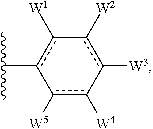

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is naphthalene, R⁴ is (CH₂)₂, R⁵ is NH, R⁶ is

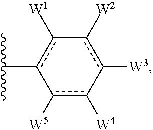

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is NH, R³ is thiophene, R⁴ is (CH₂)₂, R⁵ is NH, R⁶ is

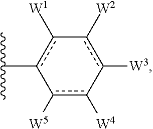

[symbol]

are single bonds, W¹, W², W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is pyridine, R⁴ is NH(CH₂)₂, R⁵ is NH, R⁶ is

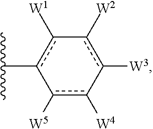

[symbol]

are single bonds, W¹, W² and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, and R⁶ is

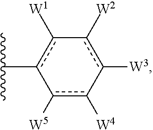

[symbol]

are single bonds, W¹, W³, and W⁵ are hydrogen, W² is methoxy, and W⁴ is 1,2-oxazolidin-2-yl)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

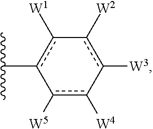

[symbol]

are single bonds, W¹, W³, and W⁵ are hydrogen, W² is chloro, and W⁴ is 1,2-oxazolidin-2-yl)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is N, R⁶ is

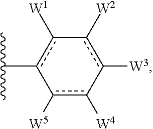

[symbol]

are single bonds, W², W⁴, and W⁵ are hydrogen, W¹ is aldehyde, and W³ is pyrimidine.

In another aspect, a compound of the disclosure comprises Formula (I), wherein A¹, A², A³, and A⁴ are hydroxyl, R¹ is (CH₂)₂, R² is absent, R³ is pyridine, R⁴ is (CH₂)₂, R⁵ is NH, R⁶ is

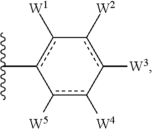

[symbol]

are single bonds, and W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is thiophene, R⁴ is (CH₂)₂, R⁵ is NH, R⁶ is

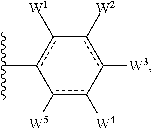

[symbol]

are single bonds, and W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is pyrazine, R⁴ is (CH₂)₂, R⁵ is NH, R⁶ is

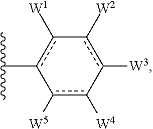

[symbol]

are single bonds, and W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is pyrimidine, R⁴ is (CH₂)₂, R⁵ is NH, R⁶ is

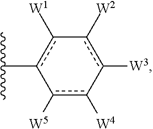

[symbol]

are single bonds, and W¹, W², and W⁴ are hydrogen, and W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is imidazole, R⁴ is (CH₂)₂, R⁵ is NH, R⁶ is

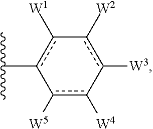

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is pyrazole, R⁴ is (CH₂)₂, R⁵ is NH, R⁶ is

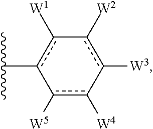

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is furan, R⁴ is (CH₂)₂, R⁵ is NH, R⁶ is

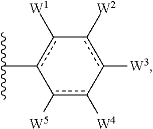

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is thiazole, R⁴ is (CH₂)₂, R⁵ is NH, and R⁶ is

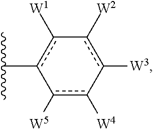

[symbol]

are single bonds, and W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

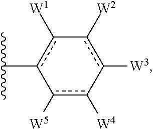

[symbol]

are single bonds, and W¹, W³, and W⁵ are hydrogen, W² is chloro, and W⁴ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

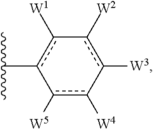

[symbol]

are single bonds, W¹, W³, and W⁵ are hydrogen, W² is methoxy, and W⁴ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

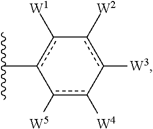

[symbol]

are single bonds, W², W⁴, and W⁵ are hydrogen, W¹ is chloro, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

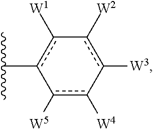

[symbol]

are single bonds, W², W⁴, and W⁵ are hydrogen, W¹ is methoxy, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

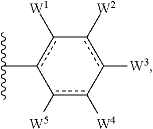

[symbol]

are single bonds, W², W⁴, and W⁵ are hydrogen, W¹ is difluoromethyl, and W³ is pyrimidine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

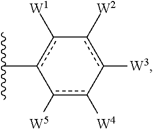

[symbol]

are single bonds, W¹, W², and W⁴ are hydrogen, W³ is azide, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

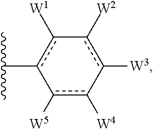

[symbol]

are single bonds, W², W⁴, and W⁵ are hydrogen, W¹ is difluoromethyl, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

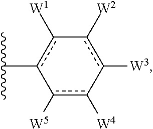

[symbol]

are single bonds, W², W⁴, and W⁵ are hydrogen, W¹ is chloro, and W³ is pyridazine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

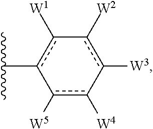

[symbol]

are single bonds, W², W⁴, and W⁵ are hydrogen, W¹ is chloro, and W³ is oxazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

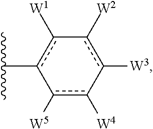

[symbol]

are single bonds, W², W⁴, and W⁵ are hydrogen, W¹ is chloro, and W³ is imidazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

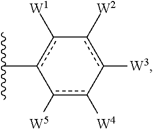

[symbol]

are single bonds, W², W⁴, and W⁵ are hydrogen, W¹ is methoxy, and W³ is pyridazine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

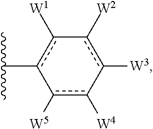

[symbol]

are single bonds, W², W⁴, and W⁵ are hydrogen, W¹ is methoxy, and W³ is oxazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

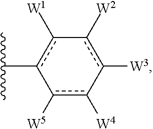

[symbol]

are single bonds, W², W⁴, and W⁵ are hydrogen, W¹ is methoxy, and W³ is imidazole.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

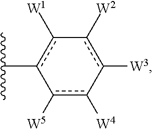

[symbol]

are single bonds, W¹, W³, and W⁵ are hydrogen, W² is pyridazine, and W⁴ is chloro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

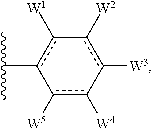

[symbol]

are single bonds, W¹, W³, and W⁵ are hydrogen, W² is oxazole, and W⁴ is chloro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

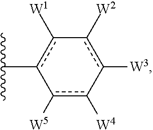

[symbol]

are single bonds, W¹, W³, and W⁵ are hydrogen, W² is pyrimidine, and W⁴ is chloro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

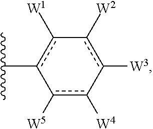

[symbol]

are single bonds, W¹, W³, and W⁵ are hydrogen, W² is imidazole, and W⁴ is chloro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

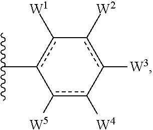

[symbol]

are single bonds, W¹, W³, and W⁵ are hydrogen, W² is pyridazine, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, and R⁶ is

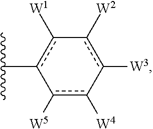

[symbol]

are single bonds, and W¹, W³, and W⁵ are hydrogen, W² is oxazole, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

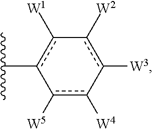

[symbol]

are single bonds, W¹, W³, and W⁵ are hydrogen, W² is pyrimidine, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

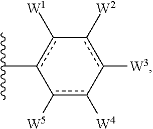

[symbol]

are single bonds, W¹, W³, and W⁵ are hydrogen, W² is isoxazole, and W⁴ is methoxy.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

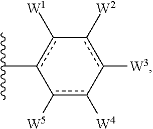

[symbol]

are single bonds, W¹, W³, and W⁵ are hydrogen, W² is imidazole, and W⁴ is methoxy.

In another aspect, a compound of the disclosure comprises Formula (I), wherein A¹, A², A³, and A⁴ are hydroxyl, R¹ is CH₂)₂, R² is absent, R³ is N(CH₂)₅OCH₃, R⁴ is (CH₂)₂, R⁵ is NH, R⁶ is

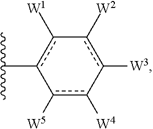

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₃, R² is absent, R³ is N(CH₂)₅OCH₃, R⁴ is (CH₂)₂, R⁵ is NH, R⁶ is

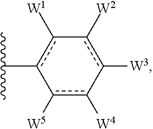

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

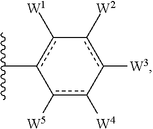

[symbol]

are single bonds, W², W⁴, and W⁵ are hydrogen, W¹ is oxazole, and W³ is (dimethylamino)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

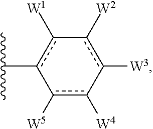

[symbol]

are single bonds, W², W⁴, and W⁵ are hydrogen, and W¹ and W³ are (dimethylamino)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

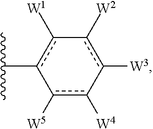

[symbol]

are single bonds, W², W⁴, and W⁵ are hydrogen, W¹ sulfonylacetamide, and W³ is (dimethylamino)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

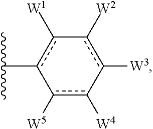

[symbol]

are single bonds, W², W⁴, and W⁵ are hydrogen, W¹ CONHAc, and W³ is (dimethylamino)methyl.

In another aspect, a compound of the disclosure comprises Formula (I), wherein A¹, A², A³, and A⁴ are hydroxyl, R¹ is (CH₂)₂, R² is absent, R³ is N(CH₂)₂CONH₃, R⁴ is (CH₂)₂, R⁵ is NH, R⁶ is

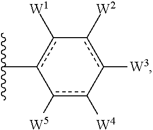

[symbol]

are single bonds, W², W⁴, and W⁵ are hydrogen, W¹ nitro, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

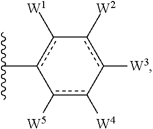

[symbol]

are single bonds, W², W⁴, and W⁵ are hydrogen, W¹ cyclopropanesulfonamide, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

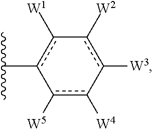

[symbol]

are single bonds, W², W⁴, and W⁵ are hydrogen, W¹ chloro, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

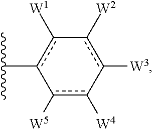

[symbol]

are single bonds, W², W⁴, and W⁵ are hydrogen, W¹ fluoro, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

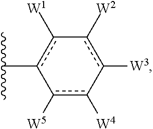

[symbol]

are single bonds, W², W⁴, and W⁵ are hydrogen, W¹ tert-butoxy, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

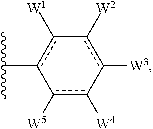

[symbol]

are single bonds, W², W⁴, and W⁵ are hydrogen, W¹ methoxy, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

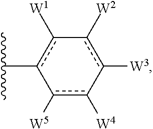

[symbol]

are single bonds, W², W⁴, and W⁵ are hydrogen, W¹ tert-butyl, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

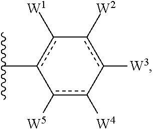

[symbol]

are single bonds, W², W⁴, and W⁵ are hydrogen, W¹ methyl, and W³ is pyrimidine.

In another aspect, a compound of the disclosure comprises Formula (I), wherein A¹, A², A³, and A⁴ are hydroxyl, R¹ is (CH₂)₂, R² is absent, R³ is N(CH₂)₃CONH₃, R⁴ is (CH₂)₃, R⁵ is NH, R⁶ is

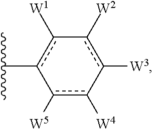

[symbol]

are single bonds, W¹, W², and W⁴ are hydrogen, W³ nitrile, and W⁵ is nitro.

In another aspect, a compound of the disclosure comprises Formula (I), wherein A¹, A², A³, and A⁴ are hydroxyl, R¹ is (CH₂)₂, R² is absent, R³ is N(CH₂)₃CONH₃, R⁴ is (CH₂)₂, R⁵ is NH, R⁶ is

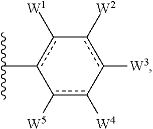

[symbol]

are single bonds, W¹, W², and W⁴ are hydrogen, W³ nitrile, and W⁵ is nitro.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

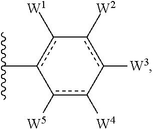

[symbol]

are single bonds, W², W⁴, and W⁵ are hydrogen, W¹ fluoro, and W³ is (dimethylamino)methyl.

In another aspect, a compound of the disclosure comprises Formula (I), wherein A¹, A², A³, and A⁴ are hydroxyl, R¹ is (CH₂)₂, R² is absent, R³ is piperazine, R⁴ is (CH₂)₂, R⁵ is NH, R⁶ is

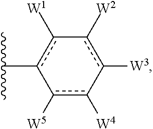

[symbol]

are single bonds, W², W⁴, and W⁵ are hydrogen, W¹ nitro, and W³ is azide.

In another aspect, a compound of the disclosure comprises Formula (I), wherein A¹, A², A³, and A⁴ are hydroxyl, R¹ is (CH₂)₂, R² is absent, R³ is 1,4-dioxane, R⁴ is (CH₂)₂, R⁵ is NH, R⁶ is

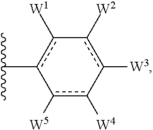

[symbol]

are single bonds, W², W⁴, and W⁵ are hydrogen, W¹ nitro, and W³ is azide.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

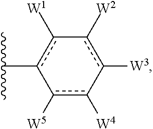

[symbol]

are single bonds, W⁴ and W⁵ are hydrogen, W¹ nitro, W² is chloro, and W³ is amine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

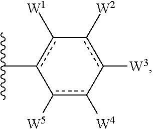

[symbol]

are single bonds, W², W⁴, and W⁵ are hydrogen, W¹ nitro, and W³ is amine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

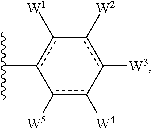

[symbol]

are single bonds, W², W⁴, and W⁵ are hydrogen, W¹ is C(NH)NH₂, and W³ is (dimethylamino)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH R⁶ is

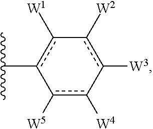

[symbol]

are single bonds, W², W⁴, and W⁵ are hydrogen, W¹ is NHCOCH₃, and W³ is (dimethylamino)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

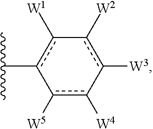

[symbol]

are single bonds, W², W⁴, and W⁵ are hydrogen, W¹ is NHCOCH₃, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

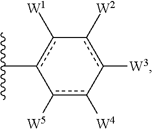

[symbol]

are single bonds, W², W⁴, and W⁵ are hydrogen, W¹ is C(NH)NH₂, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

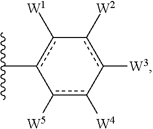

[symbol]

are single bonds, W², W⁴, and W⁵ are hydrogen, W¹ is COOH, and W³ is (dimethylamine)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

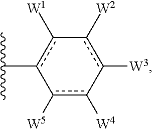

[symbol]

are single bonds, W², W⁴, and W⁵ are hydrogen, W¹ is SO₂NS(CH₃)₂, and W³ is pyrimidine.

In another aspect, a compound of the disclosure comprises Formula (I), wherein A¹, A², A³, and A⁴ are hydroxyl, R¹ is (CH₂)₂, R² is absent, R³ is CHC(O)NH₂, R⁴ is (CH₂)₂, R⁵ is NH, R⁶ is

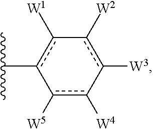

[symbol]

are single bonds, W², W⁴, and W⁵ are hydrogen, W¹ is nitro, and W³ is pyrimidine.

In another aspect, a compound of the disclosure comprises Formula (I), wherein A¹, A², A³, and A⁴ are hydroxyl, R¹ is (CH₂)₂, R² is absent, R³ is phenyl, R⁴ is (CH₂)₂, R⁵ is NH, R⁶ is

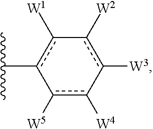

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

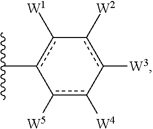

[symbol]

are single bonds, W², W⁴, and W⁵ are hydrogen, W¹ is COOH, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

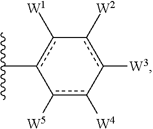

[symbol]

are single bonds, W², W³, and W⁵ are hydrogen, W¹ is cyano, and W⁴ is fluoro.

In another aspect, a compound of the disclosure comprises Formula (I), wherein A¹, A², A³, and A⁴ are hydroxyl, R¹ is absent R² is absent, R³ is azetidine, R⁴ is O(CH₂)₂, R⁵ is absent, R⁶ is

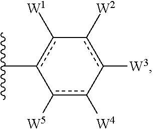

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is 1,2,3-triazole, R⁴ is (CH₂)₂, R⁵ is NH, R⁶ is

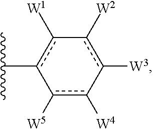

[symbol]

are single bonds, W⁴, and W⁵ are hydrogen, W¹ is nitro, W² is chloro, and W³ is nitro.

In another aspect, a compound of the disclosure comprises Formula (I), wherein A¹, A², A³, and A⁴ are hydroxyl, R¹ is (CH₂)₂, R² is absent, R³ is piperazine, R⁴ is absent, R⁵ is absent, R⁶ is

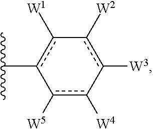

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is O, R⁴ is (CH₂)₂, R⁵ is NH, R⁶ is

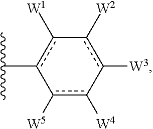

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

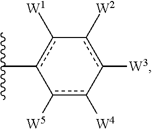

[symbol]

are single bonds, W², W⁴, and W⁵ are hydrogen, W¹ is cyano, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

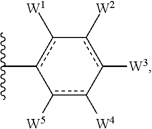

[symbol]

are single bonds, W², W⁴, and W⁵ are hydrogen, W¹ is cyano, and W³ is (dimethylamine)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

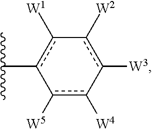

[symbol]

are single bonds, W², W⁴, and W⁵ are hydrogen, W¹ is trifluoromethyl, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

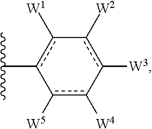

[symbol]

are single bonds, W², W⁴, and W⁵ are hydrogen, W¹ is trifluoromethyl, and W³ is (dimethylamine)methyl.

In another aspect, a compound of the disclosure comprises Formula (I), wherein A¹, A², A³, and A⁴ are hydroxyl, R¹ is (CH₂)₂, R² is absent, R³ is (CH)₂, R⁴ is (CH₂)₂, R⁵ is NH, R⁶ is

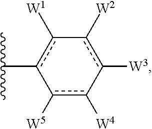

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

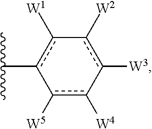

[symbol]

are single bonds, W², W⁴, and W⁵ are hydrogen, W¹ is CONHCa, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

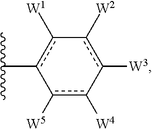

[symbol]

are single bonds, W², W⁴, and W⁵ are hydrogen, W¹ is SO₂NHCOCH₃, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

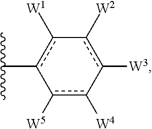

[symbol]

are single bonds, W², W⁴, and W⁵ are hydrogen, W¹ is SO₂NH₂, and W³ is (dimethylamine)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

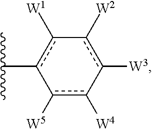

[symbol]

are single bonds, W², W⁴, and W⁵ are hydrogen, W¹ is CONH₂, and W³ is (dimethylamine)methyl.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

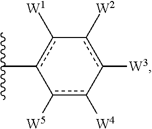

[symbol]

are single bonds, W², W⁴, and W⁵ are hydrogen, W¹ is SO₂NH₂, and W³ is pyrimidine.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

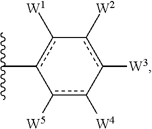

[symbol]

are single bonds, W², W⁴, and W⁵ are hydrogen, W¹ is CONH₂, and W³ is pyrimidine.

In another aspect, a compound of the disclosure comprises Formula (I), wherein A¹, A², A³, and A⁴ are hydroxyl, R¹ is (CH₂)₂, R² is absent, R³ is C(O)C(O), R⁴ is (CH₂)₂, R⁵ is NH, R⁶ is

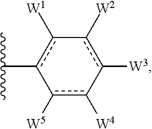

[symbol]

are single bonds, W², W⁴, and W⁵ are hydrogen, W¹ is nitro, and W is azide.

In another aspect, a compound of the disclosure comprises Formula (I), wherein A¹, A², A³, and A⁴ are hydroxyl, R¹ is (CH₂)₂, R² is absent, R³ is NHC(O)O, R⁴ is (CH₂)₂, R⁵ is NH, R⁶ is

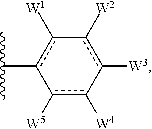

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is NH, R³ is SO₂, R⁴ is (CH₂)₂, R⁵ is NH, R⁶ is

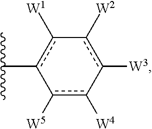

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is C(OH)C(OH), R⁴ is (CH₂)₂, R⁵ is NH, R⁶ is

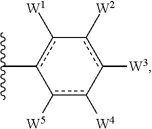

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is O, R³ is azetidine, R⁴ is (CH₂)₂, R⁵ is NH, R⁶ is

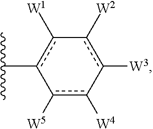

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is 1,2,3-triazole, R⁴ is (CH₂)₂, R⁵ is NH, R⁶ is

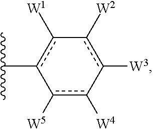

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is piperazine, R⁴ is (CH₂)₂, R⁵ is NH, R⁶ is

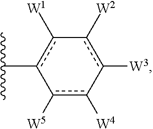

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is CH₂SO₂, R⁴ is (CH₂)₂, R⁵ is NH, R⁶ is

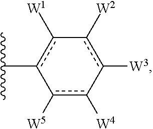

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is NHSO₂NH, R⁴ is (CH₂)₂, R⁵ is NH, R⁶ is

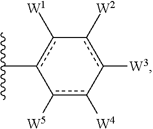

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is NHC(O)NH, R⁴ is (CH₂)₂, R⁵ is NH, R⁶ is

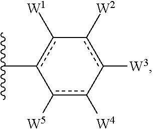

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₃, R⁴ is (CH₂)₂, R⁵ is NH, R⁶ is

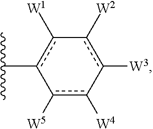

[symbol]

are single bonds, W⁴ and W⁵ are hydrogen, W¹ is nitro, W² is chloro, and W³ is azide.

In another aspect, a compound of the disclosure comprises Formula (I), wherein A¹, A², A³, and A⁴ are hydroxyl, R¹ is (CH₂)₂, R² is absent, R³ is piperazine, R⁴ is (CH₂)₂, R⁵ is NH, R⁶ is

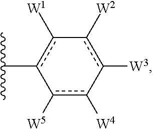

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is piperazine, R⁴ is CH₂, R⁵ is NH, R⁶ is

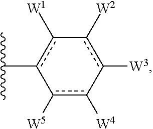

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is 1,4-dioxane, R⁴ is CH₂, R⁵ is NH, R⁶ is

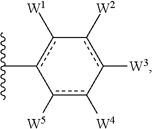

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is azetidine, R⁴ is O(CH₂)₂, R⁵ is NH, R⁶ is

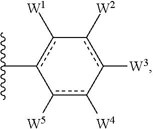

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is azetidine, R⁴ is (CH₂)₂, R⁵ is NH, R⁶ is

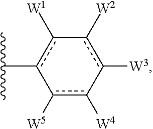

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is 1,2,3-triazole, R⁴ is (CH₂)₂, R⁵ is NH, R⁶ is

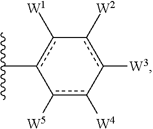

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is 1,2,3-triazole, R⁴ is CH₂, R⁵ is NH, R⁶ is

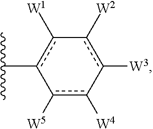

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is piperazine, R⁴ is CH₂, R⁵ is NH, R⁶ is

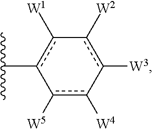

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is OCH₂, R⁴ is CH₂, R⁵ is NH, R⁶ is

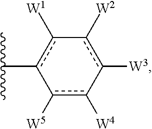

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is CH₂, R² is absent, R³ is CH₂O, R⁴ is CH₂, R⁵ is NH, R⁶ is

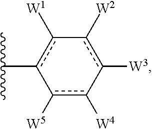

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

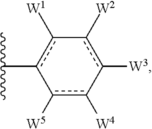

[symbol]

are single bonds, W², W⁴, and W⁵ are hydrogen, W¹ is C(O)OCH₃, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₃, R⁵ is absent, R⁶ is

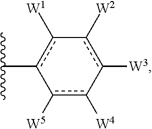

[symbol]

are single bonds, W², W⁴, and W⁵ are hydrogen, W¹ is nitro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

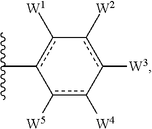

[symbol]

are single bonds, W², W⁴, and W⁵ are hydrogen, W¹ is nitro, and W³ is CCCH₃.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

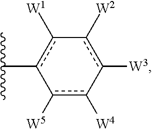

[symbol]

are single bonds, W², W⁴, and W⁵ are hydrogen, W¹ is nitro, and W³ is CH₂C(O)O(CH₂)₂CH₃.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

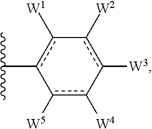

[symbol]

are single bonds, W², W⁴, and W⁵ are hydrogen, W¹ is nitro, and W³ is (CH₃)₂OH.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

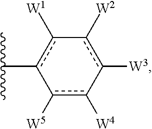

[symbol]

are single bonds, W², W⁴, and W⁵ are hydrogen, W¹ is nitro, and W³ is NHCN.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

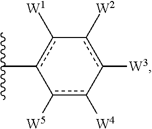

[symbol]

are single bonds, W², W⁴, and W⁵ are hydrogen, W¹ is C(NH)C(O)H, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

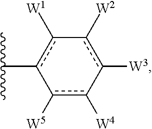

[symbol]

are single bonds, W², W⁴, and W⁵ are hydrogen, W¹ is C(NH)NH₂, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

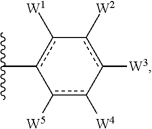

[symbol]

are single bonds, W², W⁴, and W⁵ are hydrogen, W¹ is C(NH)NH₂, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

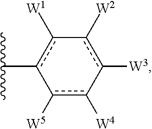

[symbol]

are single bonds, W², W⁴, and W⁵ are hydrogen, W¹ is C(O)NH₂, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

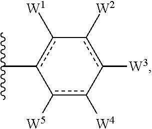

[symbol]

are single bonds, W², W⁴, and W⁵ are hydrogen, W¹ is COOH, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶

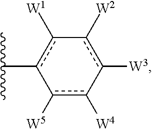

[symbol]

is are single bonds, W², W⁴, and W⁵ are hydrogen, W¹ is nitro, and W³ is CH₂CCH.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

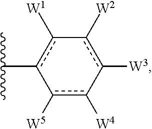

[symbol]

are single bonds, W², W⁴, and W⁵ are hydrogen, W¹ is nitro, and W³ is methylcyano.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

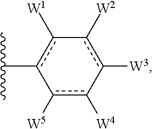

[symbol]

are single bonds, W², W⁴, and W⁵ are hydrogen, W¹ is nitro, and W³ is NHSO₂CH₃.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

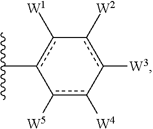

[symbol]

are single bonds, W², W⁴, and W⁵ are hydrogen, W¹ is nitro, and W³ is NCS.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

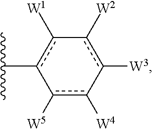

[symbol]

are single bonds, W⁴ and W⁵ are hydrogen, W¹ and W² are fluoro, and W³ is azide.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

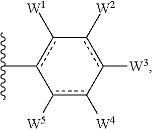

[symbol]

are single bonds, W², W⁴, and W⁵ are hydrogen, W¹ is nitro, and W³ is SCN.

In another aspect, a compound of the disclosure comprises Formula (I), wherein A¹, A², A³, and A⁴ are hydroxyl, R¹ is absent, R² is absent, R³ is (CH₂)₃, R⁴ is absent, R⁵ is absent, R⁶ is

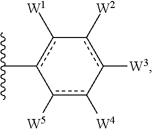

[symbol]

are single bonds, W¹, W², W⁴, and W⁵ are hydrogen, and W³ is anisole.

In another aspect, a compound of the disclosure comprises Formula (I), wherein A¹, A², A³, and A⁴ are hydroxyl, R¹ is absent, R² is absent, R³ is CH₂, R⁴ is absent, R⁵ is absent, R⁶ is

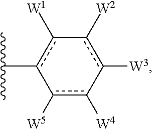

[symbol]

are single bonds, W¹, W², W⁴, and W⁵ are hydrogen, and W³ is anisole.

In another aspect, a compound of the disclosure comprises Formula (I), wherein A¹, A², A³, and A⁴ are hydroxyl, R¹ is (CH₂)₂, R² is absent, R³ is (CH₂)₃, R⁴ is absent, R⁵ is absent, R⁶ is

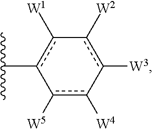

[symbol]

are single bonds, W¹, W², W⁴, and W⁵ are hydrogen, and W³ is methoxyethyl.

In another aspect, a compound of the disclosure comprises Formula (I), wherein A¹, A², A³, and A⁴ are hydroxyl, R¹ is absent, R² is absent, R³ is (CH₂)₃, R⁴ is absent, R⁵ is absent, R⁶ is

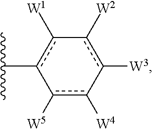

[symbol]

are single bonds, W¹, W², W⁴, and W⁵ are hydrogen, and W³ is methoxyethyl.

In another aspect, a compound of the disclosure comprises Formula (I), wherein A¹, A², A³, and A⁴ are hydroxyl, R¹ is (CH₂)₂, R² is absent, R³ is (CH₂)₃, R⁴ is (CH₂)₂, R⁵ is absent, R⁶ is

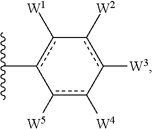

[symbol]

are single bonds, W¹, W², W⁴, and W⁵ are hydrogen, and W³ is methoxy.

In another aspect, a compound of the disclosure comprises Formula (I), wherein A¹, A², A³, and A⁴ are hydroxyl, R¹ is (CH₂)₂, R² is absent, R³ is (CH₂)₃, R⁴ is absent, R⁵ is absent, R⁶ is

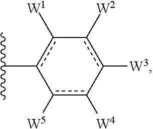

[symbol]

are single bonds, W¹, W², W⁴, and W⁵ are hydrogen, and W³ is methoxy.

In another aspect, a compound of the disclosure comprises Formula (I), wherein A¹, A², A³, and A⁴ are hydroxyl, R¹ is (CH₂)₂, R² is absent, R³ is (CH₂)₄, R⁴ is (CH₂)₂, R⁵ is absent, R⁶ is

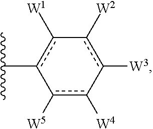

[symbol]

are absent, one CW is replaced with O, one CW is replaced with NH, and W¹, W³, and W⁴ are hydrogen.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is O, R⁶ is

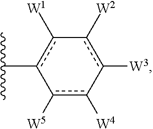

[symbol]

are single bonds, W², W³, W⁴, and W⁵ are hydrogen, and W¹ is trifluoromethyl.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

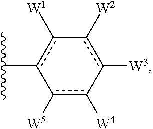

[symbol]

are single bonds, W¹, W³, and W⁵ are hydrogen, W² is methyl, and W⁴ is 2-oxa-6-azaspiro[3.4]octane.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

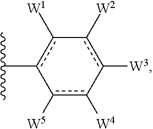

[symbol]

are single bonds, W¹, W³, and W⁵ are hydrogen, W₂ is methyl, and W⁴ is 2-oxa-7-azaspiro[3.5]nonane.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

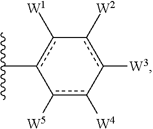

[symbol]

are single bonds, W¹, W³, and W⁵ are hydrogen, W₂ is methyl, and W⁴ is oxazole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

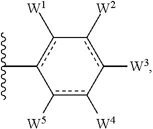

[symbol]

are single bonds, W¹, W³, and W⁵ are hydrogen, W₂ is methyl, and W⁴ is 1,2,3-triazole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

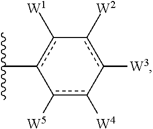

[symbol]

are single bonds, W¹, W³, and W⁵ are hydrogen, W₂ is methyl, and W⁴ is furan.

In another aspect, a compound of the disclosure comprises Formula (I), wherein A¹, A², A³, and A⁴ are hydroxyl, R¹ is CH₂, R² is absent, R³ is pyridazine, R⁴ is CH₂, R⁵ is NH, R⁶ is

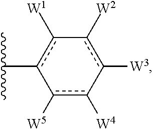

[symbol]

are single bonds, W¹, W³, and W⁵ are hydrogen, W₂ is methyl, and W⁴ is pyrimidine.

In another aspect, a compound of the disclosure comprises Formula (I), wherein A¹, A², A³, and A⁴ are hydroxyl, R¹ is CH₂, R² is absent, R³ is pyrazine, R⁴ is CH₂, R⁵ is NH, R⁶ is

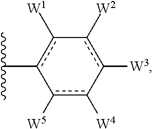

[symbol]

are single bonds, W¹, W³, and W⁵ are hydrogen, W₂ is methyl, and W⁴ is pyrimidine.

In another aspect, a compound of the disclosure comprises Formula (I), wherein A¹, A², A³, and A⁴ are hydroxyl, R¹ is CH₂, R² is absent, R³ is pyrimidine, R⁴ is CH₂, R⁵ is NH, R⁶ is

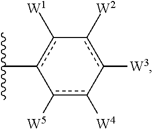

[symbol]

are single bonds, W¹, W³, and W⁵ are hydrogen, W₂ is methyl, and W is pyrimidine.

In another aspect, a compound of the disclosure comprises Formula (I), wherein A¹, A², A³, and A⁴ are hydroxyl, R¹ is CH₂, R² is absent, R³ is pyridine, R⁴ is CH₂, R⁵ is NH, R⁶ is

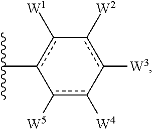

[symbol]

are single bonds, W¹, W³, and W⁵ are hydrogen, W₂ is methyl, and W⁴ is pyrimidine.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

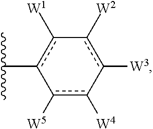

[symbol]

are single bonds, W¹, W³, and W⁵ are hydrogen, W² is methyl, and W⁴ is 1,2,3-triazole.

In another aspect, a compound of the disclosure comprises Formula (I), wherein A¹, A², A³, and A⁴ are hydroxyl, R¹ is CH₂, R² is absent, R³ is phenyl, R⁴ is CH₂, R⁵ is NH, R⁶ is

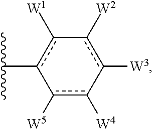

[symbol]

are single bonds, W², W⁴, and W⁵ are hydrogen, W¹ is methyl, and W³ is furan.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

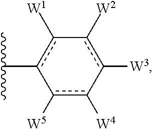

[symbol]

are single bonds, W¹ is nitro, W³ is prop-1-yn, and W², W⁴, and W⁵ are hydrogen.

In another aspect, a compound of the disclosure comprises Formula (I), wherein A¹, A², A³, and A⁴ are hydroxyl, R¹ is (CH₂)₂, R² is absent, R³ is (CH₂)₂, R⁴ is (CH₂)₂, R⁵ is NH, R⁶ is

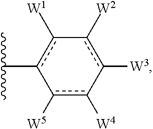

[symbol]

are single bonds, W¹ is nitro, W³ is ethan-2′-ol, and W², W⁴, and W⁵ are hydrogen.

In another aspect, a compound of the disclosure comprises Formula (I), wherein A¹, A², A³, and A⁴ are hydroxyl, R¹ is (CH₂)₅, R² is absent, R³ is CC, R⁴ is absent, R⁵ is absent, R⁶ is

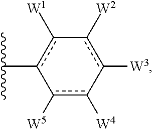

[symbol]

are single bonds, W¹ and W², W⁴, and W⁵ are hydrogen, and W³ is methoxy.

In another aspect, a compound of the disclosure comprises Formula (I), wherein A¹, A², A³, and A⁴ are hydroxyl, R¹ is (CH₂)₅, R² is absent, R³ is pyridazine, R⁴ is CH₂, R⁵ is NH, R⁶ is

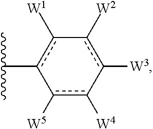

[symbol]

are single bonds, W¹, W³, and W⁵, are hydrogen, W² is methyl, and W⁴ is furan.

In another aspect, a compound of the disclosure comprises Formula (I), wherein A¹, A², A³, and A⁴ are hydroxyl, R¹ is (CH₂)₅, R² is absent, R³ is pyrazine, R⁴ is CH₂, R⁵ is NH, R⁶ is

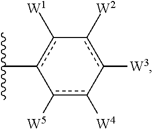

[symbol]

are single bonds, W¹, W³, and W⁵, are hydrogen, W² is methyl, and W⁴ is furan.

In another aspect, a compound of the disclosure comprises Formula (I), wherein A¹, A², A³, and A⁴ are hydroxyl, R¹ is (CH₂)₅, R² is absent, R³ is pyridazine, R⁴ is CH₂, R⁵ is NH, R⁶ is

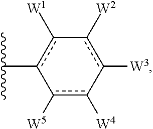

[symbol]

are single bonds, W¹, W³, and W⁵, are hydrogen, W² is cyclopropyl, and W⁴ is pyrimidine.

In another aspect, a compound of the disclosure comprises Formula (I), wherein A¹, A², A³, and A⁴ are hydroxyl, R¹ is (CH₂)₅, R² is absent, R³ is pyrazine, R⁴ is CH₂, R⁵ is NH, R⁶

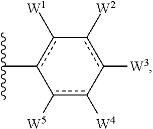

[symbol]

are single bonds, W¹, W³, and W⁵, are hydrogen, W² is methyl, and W⁴ is pyrrole.

In another aspect, a compound of the disclosure comprises Formula (I), wherein A¹, A², A³, and A⁴ are hydroxyl, R¹ is (CH₂)₅, R² is absent, R³ is pyrazine, R⁴ is CH₂, R⁵ is NH, R⁶ is

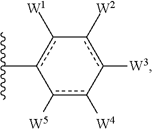

[symbol]

are single bonds, W¹, W³, and W⁵, are hydrogen, W² is methyl, and W⁴ is oxazole.

In another aspect, a compound of the disclosure comprises Formula (I), wherein A¹, A², A³, and A⁴ are hydroxyl, R¹ is (CH₂)₅, R² is absent, R³ is pyrazine, R⁴ is CH₂, R⁵ is NH, R⁶ is

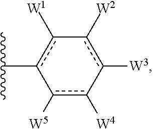

[symbol]

are single bonds, W¹, W³, and W⁵, are hydrogen, W² is methyl, and W⁴ is morpholine.

In another aspect, a compound of the disclosure comprises Formula (I), wherein A¹, A², A³, and A⁴ are hydroxyl, R¹ is (CH₂)₅, R² is absent, R³ is pyridine, R⁴ is CH₂, R⁵ is NH R⁶ is

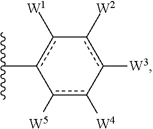

[symbol]

are single bonds, W¹, W³, and W⁵, are hydrogen, W² is cyclopropyl, and W⁴ is pyrimidine.

In another aspect, a compound of the disclosure comprises Formula (I), wherein A¹, A², A³, and A⁴ are hydroxyl, R¹ is (CH₂)₅, R² is absent, R³ is pyridine, R⁴ is CH₂, R⁵ is NH, R⁶ is

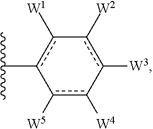

[symbol]

are single bonds, W¹, W³, and W⁵, are hydrogen, W is furan, and W is methyl.

In another aspect, a compound of the disclosure comprises Formula (I), wherein A¹, A², A³, and A⁴ are hydroxyl, R¹ is (CH₂)₅, R² is absent, R³ is pyridine, R⁴ is CH₂, R⁵ is NH, R⁶ is

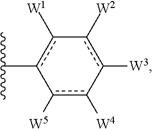

[symbol]

are single bonds, W¹, W³, and W⁵, are hydrogen, W² is pyrrole, and W⁴ is methyl.

In another aspect, a compound of the disclosure comprises Formula (I), wherein A¹, A², A³, and A⁴ are hydroxyl, R¹ is (CH₂)₅, R² is absent, R³ is pyridine, R⁴ is CH₂, R⁵ is NH, R⁶ is

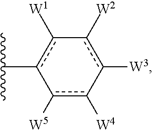

[symbol]

are single bonds, W¹, W³, and W⁵, are hydrogen, W² is morpholine, and W⁴ is methyl.

In another aspect, a compound of the disclosure comprises Formula (I), wherein A¹, A², A³, and A⁴ are hydroxyl, R¹ is (CH₂)₅, R² is absent, R³ is pyridine, R⁴ is CH₂, R⁵ is NH, R⁶ is

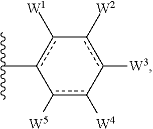

[symbol]

are single bonds, W¹, W³, and W⁵, are hydrogen, W² is oxazole, and W⁴ is methyl.
b. Compounds Comprising Formula (II)

Provided herein are compounds comprising Formula (II):

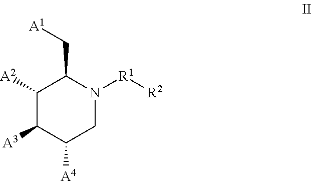

wherein
   A¹, A², A³, and A⁴ are independently selected from the group consisting of hydrogen, hydroxyl, and hydroxyl-protecting group;
   R¹ is absent or substituted or unsubstituted C₂-C₆ alkyl;
   R² is

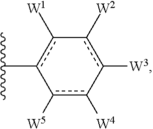

wherein optionally, at least one C or CW group in

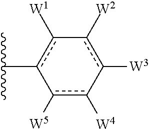

is replaced with N, O, or NH;

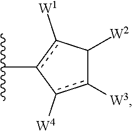

wherein optionally, at least one C or CW group in

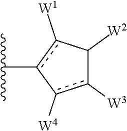

is replaced with N, O, or NH;
wherein
[symbol] are independently a single bond or are absent;
each of W¹, W², W³, W⁴, and W⁵ are independently selected from the group consisting of hydrogen, ketone, substituted or unsubstituted C₁-C₆ alkyl, and W¹ and W², W² and W³, or W³ and W⁴, W⁴ and W⁵ taken together form a substituted or unsubstituted C₅-C₆ aromatic ring;
or a pharmaceutically acceptable salt thereof.

In one aspect, a compound comprising Formula (II) comprises any of the preceding compounds of Formula (II), wherein A¹, A², A³, and A⁴ are hydroxyl.

In one aspect, a compound comprising Formula (II) comprises any of the preceding compounds of Formula (II), wherein R¹ is substituted or unsubstituted C₂-C₆ alkyl.

In a further aspect, a compound comprising Formula (II) comprises any of the preceding compounds of Formula (II), wherein R¹ is hexyl.

In one aspect, a compound comprising Formula (II) comprises any of the preceding compounds of Formula (II), wherein R² is

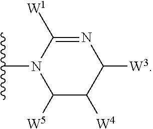

In one aspect, a compound comprising Formula (II) comprises any of the preceding compounds of Formula (II), wherein R² is

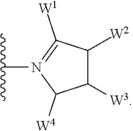

In one aspect, a compound comprising Formula (II) comprises any of the preceding compounds of Formula (II), wherein each of W¹, W², W³, W⁴, and W⁵ are independently selected from the group consisting of hydrogen, ketone, unsubstituted C₁-C₆ alkyl, and W¹ and W², W² and W³, W³ and W⁴, or W⁴ and W⁵ taken together form a substituted or unsubstituted C₅-C₆ aromatic ring.

In a further aspect, a compound comprising Formula (II) comprises any of the preceding compounds of Formula (II), wherein each of W¹, W², W³, W⁴, and W⁵ are independently selected from the group consisting of hydrogen, ketone, methyl, and W¹ and W², W² and W³, W³ and W⁴, or W⁴ and W⁵ taken together form a substituted aryl.

In another aspect, a compound comprising Formula (II) comprises any of the preceding claims of Formula (II), wherein the compound is selected from

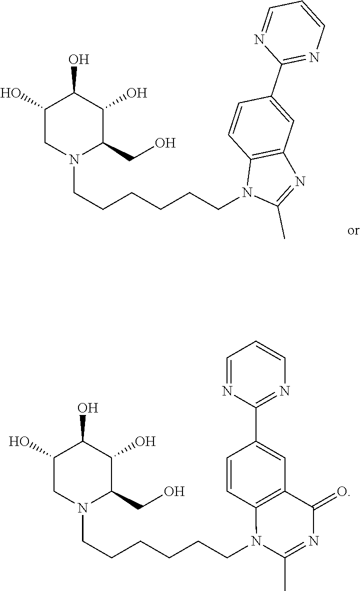 [SMILES: Cc1nc2cc(-c3ncccn3)ccc2n1CCCCCCN1C[C@H](O)[C@@H](O)[C@H](O)[C@H]1CO ; Cc1nc(=O)c2cc(-c3ncccn3)ccc2n1CCCCCCN1C[C@H](O)[C@@H](O)[C@H](O)[C@H]1CO]

In an additional aspect, a compound comprising Formula (I), wherein A¹, A², A³, and A⁴ are hydroxyl; R¹ is hexyl; wherein R² is

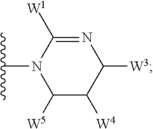

and each of W¹, W², W³, W⁴, and W⁵ are independently selected from the group consisting of hydrogen, ketone, unsubstituted C₁-C₆ alkyl, and W¹ and W², W² and W³, W³ and W⁴, or W⁴ and W⁵ taken together form a substituted or unsubstituted C₅-C₆ aromatic ring.

In an additional aspect, a compound comprising Formula (I), wherein A¹, A², A³, and A⁴ are hydroxyl; R¹ is hexyl; wherein R² is

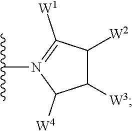

and each of W¹, W², W³, W⁴, and W⁵ are independently selected from the group consisting of hydrogen, ketone, unsubstituted C₁-C₆ alkyl, and W¹ and W², W² and W³, or W³ and W⁴, W⁴ and W⁵ taken together form a substituted or unsubstituted C₅-C₆ aromatic ring.

In another aspect, the present disclosure provides a pharmaceutically acceptable salt, hydrate, solvate, or prodrug thereof of any thereof of the compounds disclosed herein. Pharmaceutically acceptable salts include for example salts of inorganic or organic acids. In some aspects, a compound comprising Formula (I) or Formula (II), or a pharmaceutically acceptable salt, hydrate, solvate, or prodrug of any thereof, is optionally combined with one or more pharmaceutically acceptable carriers or excipients to provide a pharmaceutical composition.

c. Prodrugs

Compounds comprising Formula (I) or Formula (II) may be converted to prodrugs. One skilled in the art would recognize that certain moieties for converting one compound to a prodrug may not work for all compounds. Such prodrugs may include, but are not limited to, esters, carbonates, carbamates, phosphates, phosphonates, and the like. Additional prodrugs may be found in S. S. Dhareshwar and V. J. Stella, Prodrugs: Challenges and Rewards Part 1, Chapter: Prodrugs of Alcohols and Phenols (pp. 731-799), January 2007, DOI: 10.1007/978-0-387-49785-3_21; Kristiina M. Huttunen, Hannu Raunio and Jarkko Rautio, Prodrugs—from Serendipity to Rational Design, Pharmacological Reviews September 2011, 63 (3) 750-771, DOI: https://doi.org/10.1124/pr.110.003459; Raoul Walther, Jarkko Rautio, Alexander N. Zelikin, Prodrugs in medicinal chemistry and enzyme prodrug therapies, Advanced Drug Delivery Reviews Volume 118, 1 Sep. 2017, Pages 65-77, https://doi.org/10.1016/j.addr.2017.06.013; and Jarkko Rautio, Nicholas A. Meanwell, Li Di & Michael J. Hageman, The expanding role of prodrugs in contemporary drug design and development, Nature Reviews Drug Discovery volume 17, pages 559-587 (2018), which are incorporated by reference in their entirety as they pertain to present disclosure.

d. Pharmaceutically Acceptable Salts

The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods is prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples include, but are not limited to, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, stearic, algenic, algenic, hydroxybutyric, salicylic, galactaric, and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine), and procaine. All of these salts may be prepared by conventional means from the corresponding compound by reacting, for example, the appropriate acid or base with any of the compounds of the present disclosure.

In another aspect, the present disclosure provides processes of manufacture of the novel compounds as disclosed herein.

### III. Pharmaceutical Compositions

In another aspect, the present disclosure provides pharmaceutical compositions comprising compounds of Formula (I) or Formula (II) and at least one pharmaceutically acceptable excipient.

In some aspects, a pharmaceutically acceptable excipients include, but are not limited to, a diluent, a binder, a filler, a buffering agent, a pH modifying agent, a disintegrant, a dispersant, a preservative, a lubricant, taste-masking agent, a flavoring agent, or a coloring agent.

e. Diluent

In one aspect, the excipient is a diluent. The diluent is compressible (i.e., plastically deformable) or abrasively brittle. Suitable compressible diluents include, but are not limited to, microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, and trehalose. Suitable abrasively brittle diluents include, but are not limited to, dibasic calcium phosphate (anhydrous or dihydrate), calcium phosphate tribasic, calcium carbonate, and magnesium carbonate.

f. Binder

In another aspect, the excipient is a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

g. Filler

In another aspect, the excipient is a filler. Suitable fillers include, but are not limited to, carbohydrates, inorganic compounds, polyvinylpyrrolidone, calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and/or sorbitol.

h. Buffering Agent

In still another aspect, the excipient is a buffering agent. Suitable buffering agents include, but are not limited to, phosphates, carbonates, citrates, tris buffers, and buffered saline salts (e.g., Tris buffered saline or phosphate buffered saline).

### i. pH Modifier

In various aspects, the excipient is a pH modifier. pH modifiers include, but are not limited to, sodium carbonate, sodium bicarbonate, sodium citrate, citric acid, or phosphoric acid.

j. Disintegrant

In another aspect, the excipient is a disintegrant. The disintegrant is non-effervescent or effervescent. Non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. Suitable effervescent disintegrants may include, but are not limited to, sodium bicarbonate in combination with citric acid and sodium bicarbonate in combination with tartaric acid.

k. Dispersant

In yet another aspect, the excipient is a dispersant or dispersing enhancing agent. Suitable dispersants include, but are not limited to, starch, alginic acid, polyvinylpyrrolidones, guar gum, kaolin, bentonite, purified wood cellulose, sodium starch glycolate, isoamorphous silicate, and microcrystalline cellulose.

l. Excipient

In another alternate aspect, the excipient is a preservative. Suitable preservatives include, but are not limited to, antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

m. Lubricant

In a further aspect, the excipient is a lubricant. Suitable lubricants include, but are not limited to, minerals such as talc or silica; and fats such as vegetable stearin, magnesium stearate, or stearic acid.

n. Taste-Masking Agent

In yet another aspect, the excipient is a taste-masking agent. Taste-masking materials include, but are not limited to, cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

o. Flavoring Agent

In an alternate aspect, the excipient is a flavoring agent. Flavoring agents include, but are not limited to, synthetic flavor oils and flavoring aromatics and/or natural oils, extracts from plants, leaves, flowers, fruits, and combinations thereof.

p. Coloring Agent

In still a further aspect, the excipient is a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition is about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

q. Administration
i. Dosage Forms

The composition is formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired.

Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, P.A. (18^{th} ed, 1995), and Liberman, hours. A. and Lachman, L, Eds., Pharmaceutical Dosage Forms, Marcel Dekker Inc., New York, N.Y. (1980).

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed herein. Oral preparations may also be administered, e.g., as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds may be delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular, and intraperitoneal), the preparation is an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of a sterile liquid, for example water for injections, prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some aspects, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, e.g., an aqueous solvent. Pharmaceutical compositions adapted for topical administration in the mouth include lozenges, pastilles, and mouth washes. Transmucosal administration is accomplished through the use of, for example, nasal sprays, aerosol sprays, tablets, or suppositories, and transdermal administration is via ointments, salves, gels, patches, or creams.

r. Subject

Suitable subjects may include, but are not limited to, humans, mammals as well as companion animals such as cats, dogs, rodents, and horses; research animals such as rabbits, sheep, pigs, dogs, primates, mice, rats and other rodents; agricultural animals such as cows, cattle, pigs, goats, sheep, horses, deer, chickens and other fowl; zoo animals; and primates such as chimpanzees, monkeys, and gorillas. The subject can be of any age without limitation. In some aspects, the subject is a human.

s. Therapeutically Effective Amount

Generally, the compound comprising Formula (I) or Formula (II) will be administered in a therapeutically effective amount which includes prophylactic amounts or lower dosages for example, when combined with another agent. As used herein, “an effective amount” refers to doses of compound sufficient to provide circulating concentrations high enough to impart a beneficial effect on the recipient thereof.

The selected dose level may depend on the activity of the compound comprising Formula (I) or Formula (II), the route of administration, the severity of the condition being treated, and the condition and prior medical history of the patient being treated. In some aspects, the effective daily dose is divided into multiple doses for purposes of administration, for example, two to four doses per day. The adult human daily dosage may range from between about one microgram to about one gram, or from between about 10 mg and 100 mg, of the compound comprising Formula (I) or Formula (II) per 10-kilogram body weight. In some aspects, a total daily dose is from 0.1 mg/kg body weight to 100 mg/kg body-weight or from 1 mg/kg body weight to 60 mg/kg body weight or from 2 mg/kg body weight to 50 mg/kg body weight or from 3 mg/kg body weight to 30 mg/kg body weight. In some aspects, a daily dose is administered over one or more administering events over day. For example, in some aspects, the daily dose is distributed over two (BID) administering events per day, three administering events per day (TID) or four administering events (QID). In certain aspects, a single administering event dose ranging from 1 mg/kg body weight to 10 mg/kg body weight is administered BID or TID to a human making a total daily dose from 2 mg/kg body weight to 20 mg/kg body weight or from 3 mg/kg body weight to 30 mg/kg body weight, respectively.

### IV. Methods

The compounds discussed herein may be used for treating a number of diseases or conditions, e.g., for which inhibiting glucosidases is beneficial. Glucosidases are a class of enzymes involved in breaking down complex carbohydrates such as starch and glycogen into their respective monomers. Glucosidases include alpha-amylase, beta-amylase, gamma-amylase, cellulase, sucrase-isomaltase, mannosyl-oligosaccharide glucosidase, alpha-glucosidase, beta-glucosidase, lactase, debranching enzyme, and pullulanase. Alpha-glucosidases include maltase, glucoinvertase, glucosidosucrase, maltase-glucoamylase, alpha-glucopyranosidase, glucosidoinvertase, alpha-D-glucosidase, alpha-glucoside hydrolase, alpha-1,4-glucosidase, and alpha-D-glucoside glycohydrolase. Examples of such diseases or conditions include diabetes, diabetes mellitus type 2, viral infection (e.g., hepatitis C virus (HCV), hepatitis B virus (HBV), dengue virus (DENV), Marburg virus (MARV), Ebola virus (EBOV), Bovine Viral Diarrhea Virus (BVHV), human immunodeficiency virus (HIV), influenza A, influenza B, Japanese encephalitis virus (JEV), zika, yellow fever virus (YFV)), Pompe disease, maltase-glucoamylase deficiency, Gaucher's disease, mumps, acute pancreatitis, macroamylasemia, sucrase-isolmaltase deficiency, MOGS-CDG, celiac disease, Crohn's disease, and Cori's disease.

The compounds discussed herein may be used for treating a number of diseases or conditions, for which inhibiting ceramide glucosyltransferase and/or lowering a glycosphingolipid concentration is beneficial. Examples of such diseases or conditions include Gaucher disease (including Type I, Type II and Type III Gaucher disease), Fabry disease, Sandhoff disease, Tay-Sachs disease, Parkinson's disease, type II diabetes, hypertrophy or hyperplasia associated with diabetic nephropathy, an elevated blood glucose level, an elevated glycated hemoglobin level, a glomerular disease and lupus, including systemic lupus erythematosus. Examples of the glomerular disease include mesangial proliferative glomerulonephritis, collapsing glomerulopathy, proliferative lupus nephritis, crescentic glomerulonephritis, and membranous nephropathy.

It was recently been shown that miglustat works as a chaperon for mutated acid beta-glucosidase in cells with Gaucher disease mutations. Thus, the compounds discussed herein may be used as chaperones.

In some aspects, a disease or condition, for which inhibiting ceramide glucosyltransferase and/or lowering a glycosphingolipid concentration is beneficial, is a lysosomal glycosphinglipid storage disease (LSD), such as Gaucher (types I, II, and III) disease, Fabry disease, Sandhoff disease, Tay-Sachs disease, GM1 Gangliosidosis, and Niemann-Pick Type C disease.

In some respects, a disease or condition, for which inhibiting ceramide glucosyltransferase and/or lowering a glycosphingolipid concentration is beneficial, is multiple myeloma. In addition, inhibition of osteoclastogenesis and/or reducing osteoclast activation associated with multiple myeloma may utilize an iminosugar, which may be a ceramide glucosyltransferase inhibitor or a glucosidase inhibitor. In some respects, a disease or condition, for which inhibiting ceramide glucosyltransferase and/or lowering a glycosphingolipid concentration is beneficial, is osteoporosis or osteoarthritis. Inhibition of osteoclastogenesis and/or reducing osteoclast activation associated with these disorders will prevent bone resorption.

In some respects, a disease or condition, for which inhibiting ceramide glucosyltransferase and/or lowering a glycosphingolipid concentration is beneficial, is polycystic kidney disease, including an autosomal dominant or recessive form of the polycyctic kidney disease. In some respects, a disease or condition, for which inhibiting ceramide glucosyltransferase and/or lowering a glycosphingolipid concentration is beneficial, may atherosclerosis or renal hypertrophy in a diabetic patient.

In some respects, a disease or condition, for which inhibiting ceramide glucosyltransferase and/or lowering a glycosphingolipid concentration is beneficial, is Type II diabetes and/or its related disease or condition. In some respects, such disease or condition is a non-alcoholic fatty liver disease, which is a consequence of the metabolic syndrome and type II diabetes. In some respects, the related disease or condition is a metabolic syndrome and/or associated dyslipidemia, which is a precursor of type II diabetes and/or atherosclerosis. In some respects, the compounds discussed herein may be used prophylactically for the prevention of Type II diabetes and/or its related disease or condition. Although the present disclosure is not limited by any theory, the inventors hypothesize that the rationale for the treatment and/or prevention of Type II diabetes and/or its related disease or condition is that a compounds discussed herein may reduce the concentration of glucosylceramide also reduces the expression of gangliosides, especially GM3, which may result in the engagement of insulin receptor into lipid rafts, causing receptor inactivation and internalization resulting in insulin resistance. The compounds discussed herein may therefore deplete cells of surface GM3 and sensitize the cells to insulin, thereby being useful in the treatment of insulin resistance, which is central to the development of, for example, metabolic syndrome, type II diabetes, non-alcoholic liver disease and atherosclerosis.

In some respects, the compounds discussed herein may be used for the treatment of a bacterial diseases caused by a toxin, which binds through or to glycosphingolipid or ganglioside. For example, cholera is caused by a toxin (cholera toxin) that binds via its B-subunit to ganglioside GM1. By of a cholera patient, e.g., orally or by colonic irrigation, with an iminosugar, the expression of the GM1 target by susceptible cells in the gut epithelium is abolished or substantially reduced, having a corresponding therapeutic effect by reducing the effect of the toxin. Another disease involving bacterial toxins is postdiarrhea hemolytic uremic syndrome, which is commonly associated with particular strains of E. coli bacteria that produce Shiga toxin type-2 which binds to the ganglioside globotriaosylceramide (Gb3). By analogy to the scenario above described for cholera therapy, the compounds comprising Formula (I) or Formula (II) disclosed above may be used to treat E. coli—associated disorders by reducing cellular expression of the ganglioside target of the toxin (in this case Gb3). Shiga toxin-2 is commonly expressed by E. coli O157:H7 which is a strain of E. coli known to cause enterohemorrhagic disease. The compounds comprising Formula (I) or Formula (II) disclosed above may be used therefore to treat enterohemorrhagic disease associated with 0157, but also enterohemorrhagic disease caused by other bacteria that express Shiga toxin-2.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred examples of modes for its practice. However, those of skill in the art should, in light of the present disclosure, appreciate that many changes can be made in the specific examples which are disclosed and still obtain a like or similar result without departing from the spirit and scope of the disclosure.

¹H NMR spectra were reported in ppm from tetramethylsilane (TMS) on the S scale. Data are reported as follows: chemical shift, multiplicity (s=singlet, d=doublet, t=triplet, m=multiplet, complex multiplets used where overlapping multiplets are not resolved, br=broadened, when spin systems are distorted due to non-first order effects), coupling constants (Hz), and assignments or relative integration where appropriate.

For all examples describing synthetic schemes below, intermediate and product identity was confirmed by proton nuclear magnetic resonance spectroscopy (¹H-NMR) and/or liquid chromatography-mass spectrometry (LCMS). Product purity was confirmed by LCMS and high-performance liquid chromatography (HPLC) with a target purity of ≥approximately 90%. Purity of intermediates was suitable for the intended use. In selected cases (for examples where the intermediate was considered likely to be labile or where subsequent purification was expected to provide suitable material) intermediate after workup was moved forward to the next reaction without further purification. References to purification on silica, by column, or by chromatography, unless otherwise specified refer to purification by column chromatography using silica gel (100-200 or 60-120 mesh) with the indicated eluent. References to evaporation, or removal or concentration of reaction or volatiles or solvent, unless otherwise specified refer to solvent removal under reduced pressure using a diaphragm vacuum pump and ROTAVAPOR system. References to purification by preparative HPLC unless otherwise specified indicate a KINETEX Evo reverse phase C18 column (5 μm, 250 mm×21.2 mm), with an acetonitrile-water slow gradient and 5 mM ammonium bicarbonate buffer. For some intermediates and products more than one batch was prepared and combined where necessary to provide the desired amounts. Reaction endpoints were determined by thin layer chromatography on silica. The Example below provide representative conditions and scales for single batches. Weights of intermediates and products are approximate. Room temperature (rt) is approximately 20° C. to 35° C.

### Example 1: Synthesis of TBS-DNJ and Int-1

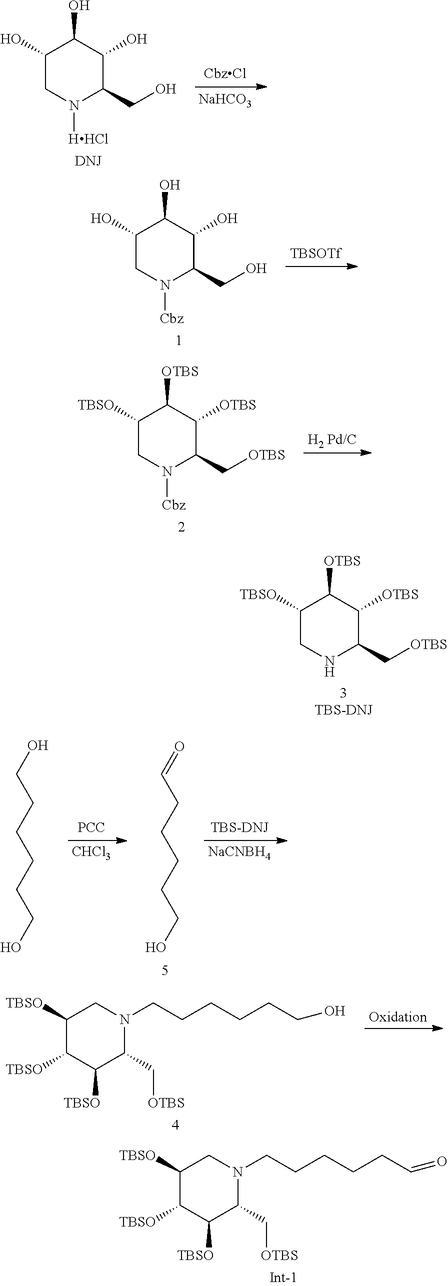

Preparation of TBS-DNJ and Int-1 have been previously reported. In brief, to a stirred solution of (2R,3R,4R,5S)-2-(hydroxymethyl)piperidine-3,4,5-triol hydrochloride (DNJ, 25 g) in saturated NaHCO₃ solution (300 mL) was added 1 equivalent (eq) benzyl chloroformate (50% in toluene, 42.8 mL, 125.62 mmol) drop wise at 0° C. The reaction mixture was warmed to room temperature and stirred for 6 hours. The reaction mixture was diluted with water (500 mL), washed with dichloromethane (DCM, (3 times with 300 mL) and separated. The aqueous layer was extracted with ethyl acetate, (EtOAc, 5×300 mL). The organic extracts (combined) were dried over sodium sulphate (Na₂SO₄), filtered, and concentrated in vacuo to afford 1 (30 g, Cbz-DNJ) as thick syrup.

To a stirred solution of 1 (30 g, 101.10 mmol) in chloroform (CHCl₃, 600 mL) were added tert-butyldimethylsilyl (TBS) trifluoromethanesulfonate (139.6 mL, 606.06 mmol) and 2,6-lutidine (117.5 mL, 1011.00 mmol) at 0° C. under Ar atmosphere. The reaction mixture was warmed to room temperature and stirred 16 hours. The reaction was diluted with water (500 mL) and extracted with CH₂Cl₂ (3×300 mL). The DCM extracts were dried over sodium sulphate, filtered, concentrated, and the material purified on silica with 3% EtOAc-hexane to afford 2 (50 g) as colorless thick syrup which was used as-in in subsequent steps.

Preparation of 3 (TBS-DNJ): To a stirred solution of 2 (50 g) in EtOAc (500 mL) added 10% Pd/C (10 g, 50% wet) at room temperature. The reaction mixture was kept under H₂ atmosphere (balloon pressure), stirred for 24 hours then filtered through a pad of CELITE. The filtrate was concentrated and purified on silica with 3% EtOAc-hexane to afford TBS-DNJ (41.6 g) as thick syrup.

Preparation of Int-1: Stirred hexane-1,6-diol (50 g) in CHCl₃ (1 L), added pyridinium chlorochromate (PCC, 55 g) and CELITE (50 g) at room temperature under Ar, stirred 3 hours. The reaction mixture was filtered, concentrated, and purified on silica [40% EtOAc-hexane] to afford 5 (10 g) as thick syrup.

To a stirred solution of TBS-DNJ (40 g) in methanol (MeOH, 800 mL) added 5 (9.05 g) and catalytic amount (cat.) of acetic acid at 0° C. under Ar atmosphere and stirred 30 minutes. The reaction mixture was warmed to room temperature for 15 minutes, added sodium cyanoborohydride (NaCNBH₃, 6.09 g) and stirred for 16 hours. The volatiles were removed and the residue was diluted with water (200 mL) and extracted with EtOAc (3×200 mL). Organic extracts were dried over sodium sulphate, filtered, concentrated, and the material was purified on silica with 10% EtOAc-hexane to afford 4 (35 g) as colourless thick syrup.

To a stirred solution of oxalyl chloride ((COCl)₂, 3.57 mL) in tetrahydrofuran (THF, 200 mL) was added dimethyl sulfoxide (DMSO, 3.57 mL) at −78° C. under Ar atmosphere and stirred for 15 minutes. To this was added 4 (14 g) in THE (30 mL) drop wise at −78° C. and stirred for 30 minutes. Then triethylamine (10.65 mL) was added at −78° C. and gradually warmed to room temperature for 1 hour. The reaction was quenched with ice cold water (100 mL) and extracted with EtOAc (3×100 mL). Organic extracts were dried over Na₂SO₄, filtered, concentrated, and the material purified on silica [10% EtOAc-hexane] to afford Int-1 (10 g) as colorless syrup used immediately in subsequent steps.

Preparation of tetra-O-acetyl-DNJ: room temperature (Cbz-DNJ, 15.0 g), CHCl₃ (300 mL), pyridine (12.0 eq), cooled to 0° C., added Ac₂O (10.0 eq) then reacted at room temperature 72 hours. Reaction was diluted with water ice-cold water (200 mL) and extracted with DCM (2×200 mL). The organic layer was dried over anhydrous Na₂SO₄ and concentrated then purified on silica [30% EtOAc:hexane] to afford 20 g of the tetraacetyl analog of 2. To this material in EtOAc (400 mL) added 10% Pd/C (50% wet, 10 g), reacted under H₂ (balloon pressure) at room temperature for 36 hours. Reaction mass was filtered through a CELITE bed and washed with EtOAc. The filtrate was concentrated under reduced pressure to afford 15 g of tetra-O-acetyl DNJ (Ac-DNJ, not shown in figure) as colorless thick syrup.

Preparation of 4-fluoro-3-nitrophenyl azide (not shown in figure): 4-Fluoro-3-nitroaniline (50 g), NaN₃ (3 eq), t-BuOH:water (500 mL:100 mL) mixed at 0° C. then t-butyl nitrite (17 eq) was added and stirred at 55° C. for 3 hours. The reaction mass was diluted with water and extracted with DCM. The organic layer was washed with brine solution, dried over anhydrous Na₂SO₄, concentrated, and purified on silica [4% EtOAc in hexane] to afford 30 g of 4-fluoro-3-nitrophenyl azide (1-fluoro-2-nitro-4-azidobenzene, FNAB).

### Example 2: Synthesis of Int-2

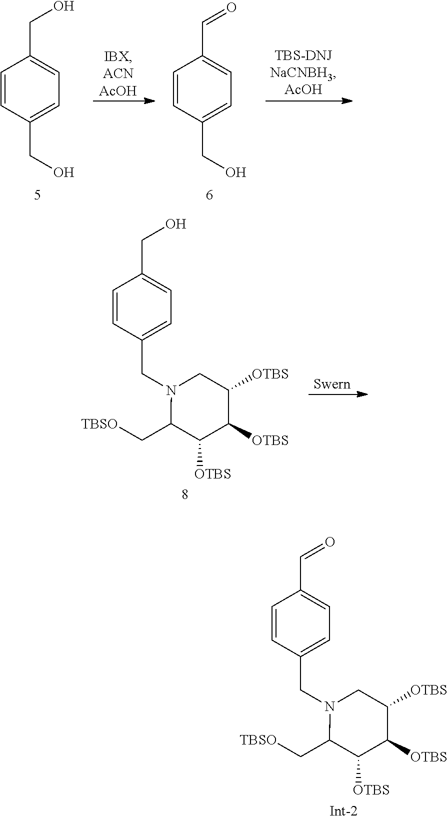

1,4-Phenylenedimethanol (250 g) in acetonitrile (ACN, 7.5 L), mixed at 0° C., added 2-iodoxybenzoic acid (IBX) (0.7 eq), AcOH (1.0 eq), raised to room temperature. After 16 hours the reaction was filtered through CELITE bed and washed with EtOAc (6 L). The filtrate was concentrated, the residue was dissolved in EtOAc (4 L) and washed with saturated aqueous NaHCO₃ solution. The organic layer was dried over Na₂SO₄, filtered, concentrated, and purified on silica [20% EtOAc:hexane] to afford 100 g of 6.

6 (100 g) was mixed with MeOH:DCM (4 L: 0.5 L) at 0° C. Added TBS-DNJ (0.6 eq), AcOH (1 mL), NaCNBH₃ (1.5 eq) and stirred at room temperature. After 16 hours the reaction was distilled under reduced pressure. The obtained material was dissolved in EtOAc (3 L) and washed with water and dried over Na₂SO₄, filtered, concentrated, and purified on silica eluting with 15% EtOAc/hexane to afford 220 g of 8.

(COCl)₂ (3.0 eq) and DCM (100 mL) were mixed at −78° C. followed by addition of DMSO (4.0 eq) over 30 minutes then addition of 8 (10 g) in DCM (100 mL). Reaction was maintained at −78° C. for 1 hour then quenched with triethylamine (Et₃N or TEA, 5.0 eq). Raised to room temperature with stirring for 2 hours. The reaction was diluted with water (150 mL) and extracted with DCM (2×200 mL). The combined organic layer was washed with water and dried over anhydrous Na₂SO₄, filtered, concentrated, and purified on silica eluting with 3% EtOAc/hexane to obtain 7.5 g Int-2 as colorless syrupy liquid.

### Example 3: Synthesis of Int-3 and Compound 1033 ((2R,3R,4R,5S)-2-(hydroxymethyl)-1-{[3-({[3-methyl-5-(1H-pyrrol-2-yl)phenyl]amino}methyl)phenyl]methyl}piperidine-3,4,5-triol)

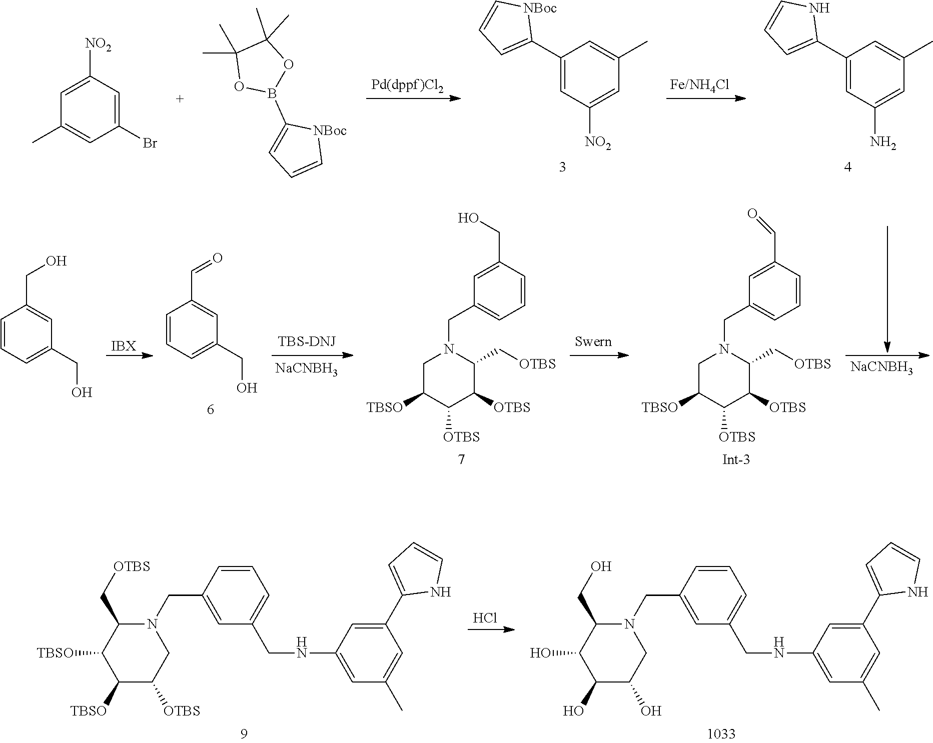

1-Bromo-3-methyl-5-nitrobenzene (30 g), 1-Boc-pyrrole-2-boronate (for convenience, 4,4,5,5-tetramethyl-1,3,2-dioxaborolanes are referred to as boronic acid pinacol esters or boronates) (1.5 eq), toluene:EtOH:water (1:1:1, 900 mL), Na₂CO₃ (3.0 eq), degassed with N₂ for 20 minutes, added Pd(dppf)Cl₂ (0.1 eq) and increased to 80° C. After 16 hours volatiles were removed, the residue was diluted with water and EtOAc, filtered through a bed of CELITE. The organic layer was separated and dried over anhydrous Na₂SO₄, filtered, concentrated, and purified by COMBIFLASH with 5% EtOAc/hexane to afford 36 g of 3 as thick syrup.

3 (24 g), EtOH:H₂O (1:1, 480 mL), Fe (10 eq), NH₄Cl (10 eq), mixed at room temperature then increased to 100° C. After 16 hours reaction was filtered through a pad of CELITE, concentrated, and the residue diluted with water and EtOAc. The organic layer was separated and dried over anhydrous Na₂SO₄, filtered, concentrated, and purified by COMBIFLASH with 30% EtOAc/hexane to provide 10 g of 4 as ash-color solid.

1,3-Benzenedimethanol (200 g), IBX (0.5 eq), acetic acid (AcOH, 1.0 eq), ACN (6 L) mixed at 0° C. then warmed to room temperature. After 16 hours the reaction was filtered through a pad of CELITE. Filtrate volume was reduced, the material was diluted with EtOAc and washed with aqueous saturated sodium carbonate (NaHCO₃) solution. The organic layer was separated and dried over anhydrous Na₂SO₄, filtered, concentrated, and purified by chromatography eluting with 30% EtOAc/hexane to obtain 90 g of 6 as pale yellow syrup.

6 (80 g, 1.5 eq), MeOH (200 mL), TBS-DNJ (1 eq), AcOH (cat.) was stirred 10 minutes at room temperature, added NaCNBH₃ (1.5 eq) at 0° C. then raised to room temperature. After 16 hours volatiles were removed, mass was diluted with water (2 L), extracted with EtOAc (2×3 L). The organic layer was washed with water, dried over anhydrous Na₂SO₄, filtered, concentrated, and purified on silica [5% EtOAc/hexane] to obtain 250 g of 7 as yellow syrup.

(COCl)₂ (3.0 eq) and DCM (1.5 L) were mixed at −78° C., added DMSO (4.0 eq) over 30 minutes followed by 7 (75 g) and DCM (1.5 mL). Stirred at −78° C. for 1 hour then quenched with TEA (5.0 eq). Raised to room temperature, stirred 2 hours diluted with water (2 L), extracted with DCM (2×500 mL). The organic layer was washed with water, dried over anhydrous Na₂SO₄, filtered, concentrated, and purified on silica column chromatography eluting with 5% EtOAc/hexane to obtain 52 g of Int-3 as pale yellow syrup.

Int-3 (104 g), MeOH/DCM (1:1, 2 L), 4 (24 g, 1.0 eq), AcOH (cat.) mixed 10 minutes at room temperature, added NaCNBH₃ (2 eq) at 0° C. then increased to room temperature. After 24 hours reaction mixture was concentrated, residue diluted with water and extracted with EtOAc which was dried over Na₂SO₄, filtered, and concentrated. The product mixture was retreated with MeOH/DCM (1:1, 2 L) and added 1.0 eq NaCNBH₃ under N₂ atmosphere and stirred for another 16 hours. The reaction was concentrated, residue was diluted with water (2 L) and extracted with EtOAc (2×2 L). The organic layer was washed with water and dried over anhydrous Na₂SO₄, filtered, concentrated, and the material purified by COMBIFLASH [5% EtOAc/hexane] to obtain 40 g of 9 as colorless to pale pink syrup.

9 (15 g), MeOH:DCM (1:1, 300 mL), 4.0 M HCl in 1,4-dioxane (150 mL) mixed at 0° C. then increased to room temperature. After 2 hours the mixture was concentrated, residue was dissolved in water and basified with NaHCO₃ and extracted with 10% MeOH/EtOAc (500 mL). The organic layer was dried over anhydrous Na₂SO₄, filtered, concentrated, and purified by COMBIFLASH with 5% MeOH/EtOAc to provide 6 g of 1033 as off-white solid. Combined batches were pooled and lyophilized followed by high vacuum drying and 20.5 g 1033 was obtained.

### Example 4: Synthesis of Int-4

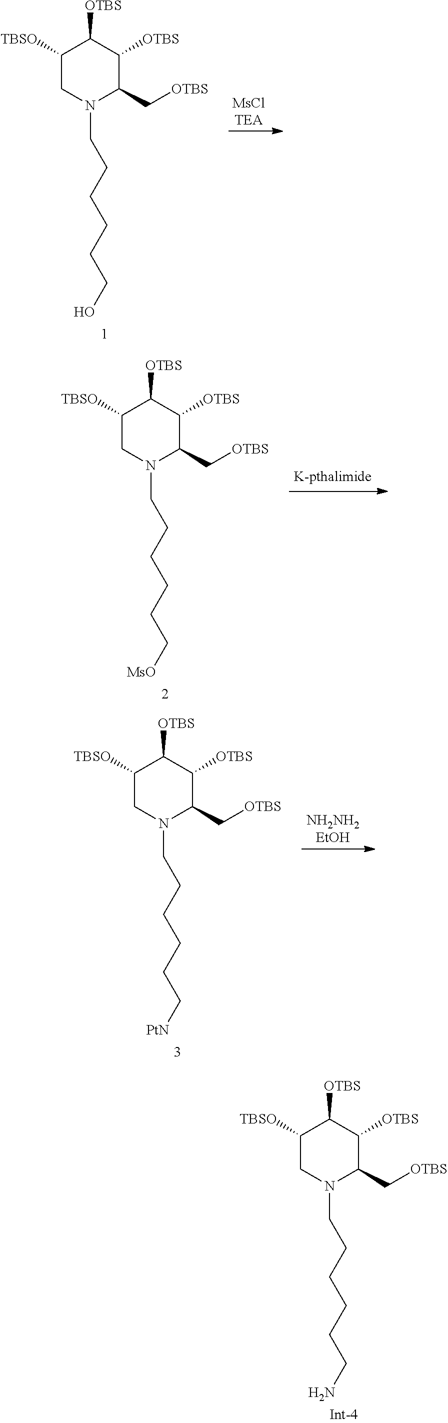

1 (prepared as in Example 1, 50 g), DCM (500 mL), Et₃N (3.0 eq), MsCl (1.3 eq) mixed at room temperature for 30 minutes. Reaction was quenched with ice-cold water (1 L) and extracted with DCM (2×1 L). The organic layer was washed with water (2×500 mL) and dried over Na₂SO₄, filtered, concentrated to afford 2 (50 g).

2 (50 g), DMF (500 mL), potassium phthalamide (1.3 eq) mixed at room temperature then maintained at 80° C. After 12 hours reaction was quenched with ice-cold water (1 L) and extracted with EtOAc (2×500 mL). The organic layer was dried over Na₂SO₄, filtered, concentrated, and purified on silica [5% EtOAc/hexane] to provide 16 g of 3.

3 (16 g), EtOH (200 mL), N₂H₄·H₂O (75% 1.0 N water) (5 eq) mixed at room temperature for 16 hours. The reaction was concentrated. The residue was quenched with ice-cold water (2 L), extracted with EtOAc (2×500 mL) and the organic layer was dried over Na₂SO₄, filtered, concentrated to afford 12 g of Int-4.

### Example 5: Synthesis of 1012 ((2R,3R,4R,5S)-1-{[4-({[3-cyclopropyl-5-(pyridazin-3-yl)phenyl] amino}methyl)phenyl]methyl}-2-(hydroxymethyl)piperidine-3,4,5-triol) and 1001 ((2R,3R,4R,5S)-1-{[4-({[3-cyclopropyl-5-(pyridazin-3-yl)phenyl] (methyl)amino}methyl)phenyl]methyl}-2-(hydroxymethyl)piperidine-3,4,5-triol)

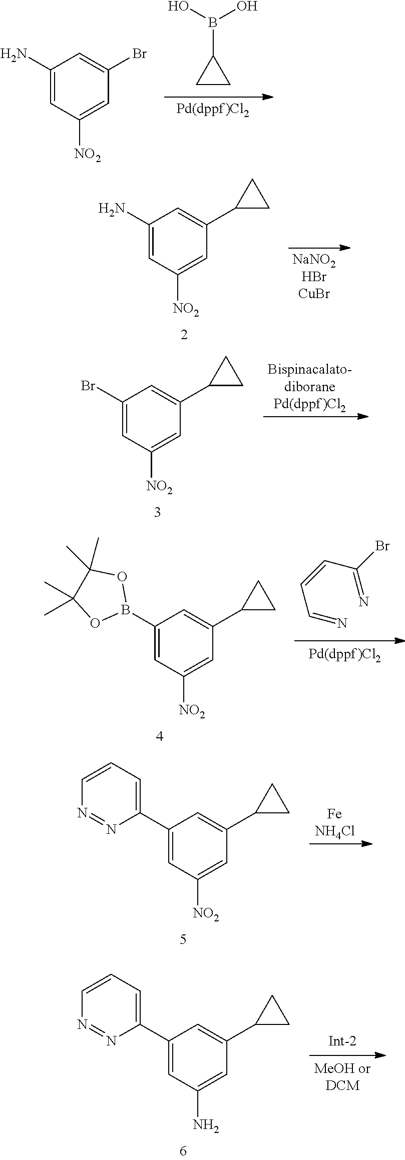

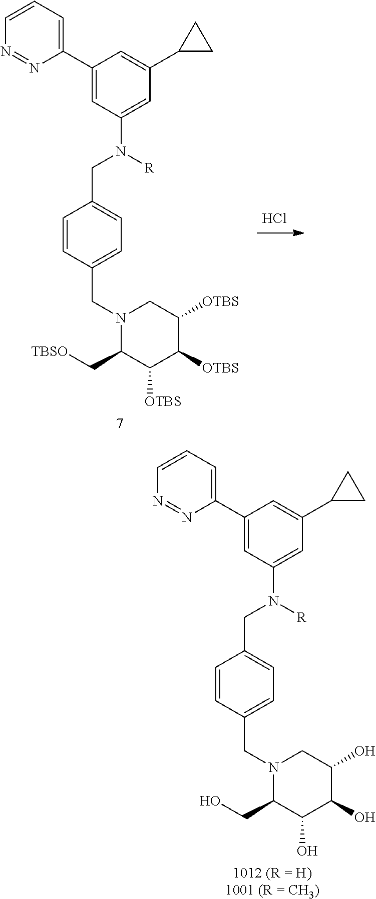

3-Bromo-5-nitroaniline (8 g), 1,4-dioxane (160 mL), cyclopropyl boronic acid (1.5 eq), Cs₂CO₃ (2.0 eq), degassed 15 minutes with N₂, Pd(dppf)Cl₂ in DCM (0.1 eq) addition at room temperature then increased to 100° C. for 16 hours. The reaction was concentrated, the residue was diluted with EtOAc and water, filtered through a pad of CELITE and washed with EtOAc. Combined organic layers were washed with water, dried over Na₂SO₄, concentrated, and purified on silica [20-30% EtOAc/hexane] to afford 2 (6 g) as yellow solid.

2 (4 g), 48% aq. HBr (200 mL), NaNO₂ (1.5 eq) at −10° C. mixed 30 minutes then CuBr (5 eq) and H₂O (240 mL) added at 0° C. After 16 hours reaction was diluted with water and extracted with EtOAc, organic layer was washed with water, dried over anhydrous Na₂SO₄, filtered, concentrated, and purified on silica with hexane to afford 3 (3 g).

An alternate preparation of 3 based on literature used 3,5-dibromo-nitrobenzene and cyclopropyl boronic acid as starting materials. This approach yielded a mixture of mono- and di-cyclopropyl-nitrobenzenes which was used as-is for initial preparation of 1012 and 1001 however the approach for which details are provided above yielded predominantly the mono-cyclopropyl intermediate.

A mixture of 3 (3.5 g), 1,4-dioxane (70 mL), bis(pinacolato) diboron (1.5 eq), KOAc (3.0 eq) was degassed with N₂ for 15 minutes, added Pd(dppf)Cl₂ (0.2 eq), reacted 4 hours at 100° C. Reaction was diluted with water, extracted with EtOAc, the organic layer was washed with water, dried over anhydrous Na₂SO₄, filtered, and concentrated to afford 3.5 g of 4.

A mixture of 4 (3.5 g), toluene:H₂O:EtOH (70 mL, 1:1:1), 3-bromo-pyridazine (1.1 eq), Na₂CO₃ (3.0 eq) was degassed for 15 minutes with N₂, then Pd(dppf)Cl₂ (0.1 eq) added at room temperature and mixture raised to 90° C. After 16 hours the reaction was diluted with water and extracted with EtOAc. The organic layer was washed with water, dried over anhydrous Na₂SO₄, filtered, concentrated, and purified on silica [50% EtOAc/hexane] to afford 1.5 g of 5.

5 (1.4 g), EtOH:H₂O (40 mL, 2:1), Fe (2 eq), NH₄Cl (4.0 eq) were mixed by addition at room temperature then increased to 90° C. After 16 hours the reaction was diluted with water and extracted with EtOAc. The organic layer was washed with water, dried over anhydrous Na₂SO₄, filtered, and concentrated to afford 600 mg of 6 as colorless thick syrup.

Preparation of 1001 ((2R,3R,4R,5S)-1-{[4-({[3-cyclopropyl-5-(pyridazin-3-yl)phenyl](methyl)amino}methyl)phenyl]methyl}-2-(hydroxymethyl)piperidine-3,4,5-triol): Int-2 (Example 2, 1.2 g), MeOH (80 mL), 6 (1.0 eq), AcOH (0.2 mL), NaCNBH₃ (1.5 eq) were stirred at room temperature. After 16 hours solvent was removed and the reaction mass was diluted with water (40 mL) and extracted with DCM (2×40 mL). The organic layer was washed with water and dried over anhydrous Na₂SO₄ then concentrated and purified on silica with 10-40% EtOAc-hexane to afford 500 mg of 7 with R═CH₃.

7 with R═CH₃ (500 mg), DCM (10 mL), 4.0 M HCl in 1,4-dioxane (5 mL) were mixed by addition at 0° C. then temperature was raised to room temperature. After 16 hours volatiles were removed and the residue triturated with Et₂O (30 mL) and EtOAc (30 mL) to afford 150 mg of 1001 as the HCl salt as yellow solid.

Preparation of 1012 ((2R,3R,4R,5S)-1-{[4-({[3-cyclopropyl-5-(pyridazin-3-yl)phenyl]amino}methyl)phenyl]methyl}-2-(hydroxymethyl)piperidine-3,4,5-triol): Stirred Int-2 (400 mg), DCM (10 mL), 6 (1.0 eq), AcOH (0.2 mL), NaCNBH₃ (1.5 eq) at room temperature. After 16 hours solvent was removed and the residue was diluted with water (20 mL) and extracted with DCM (2×10 mL). The organic layer was washed with water and dried over anhydrous Na₂SO₄, concentrated, and purified on silica with 10-40% EtOAc/hexane to afford 150 mg of 7 with R═H as colorless thick syrup.

7 (150 mg), DCM (4 mL), 4.0 M HCl in 1,4-dioxane (1.5 mL) were mixed at 0° C. then raised to room temperature. After 16 hours reaction volatiles were concentrated and residue triturated with Et₂O (20 mL) and EtOAc (10 mL) to obtain 100 mg of 1012 as yellow solid.

### Example 6: Synthesis of Compound 1003 ((2R,3R,4R,5S)-2-(hydroxymethyl)-1-[6-({3-methyl-5-[(morpholin-4-yl)methyl]phenyl}amino)hexyl]piperidine-3,4,5-triol)

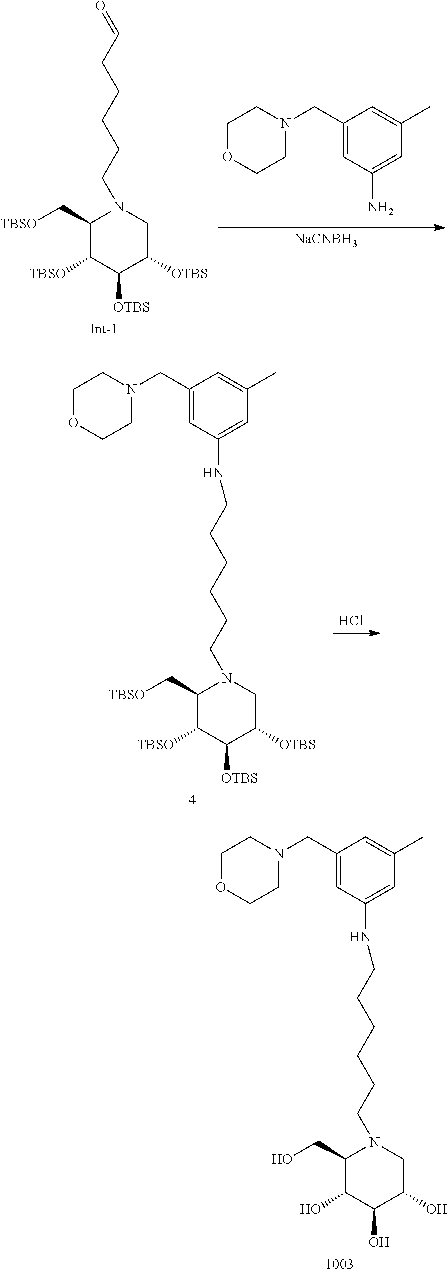 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCCCCC=O ; Cc1cc(N)cc(CN2CCOCC2)c1 ; "NaCNBH3" ; Cc1cc(CN2CCOCC2)cc(NCCCCCCN2C[C@H](O[Si](C)(C)C(C)(C)C)[C@@H](O[Si](C)(C)C(C)(C)C)[C@H](O[Si](C)(C)C(C)(C)C)[C@H]2CO[Si](C)(C)C(C)(C)C)c1 ; Cl ; Cc1cc(CN2CCOCC2)cc(NCCCCCCN2C[C@H](O)[C@@H](O)[C@H](O)[C@H]2CO)c1]

Int-1 (2.5 g, Example 1) and DCM were mixed with 3-methyl-5-(4-morpholinylmethyl)-benzenamine (Example 7, 1 eq) at 0° C. followed by addition of AcOH (0.2 mL) and NaCNBH₃ (1.5 eq). Temperature was raised to room temperature. After 16 hours the reaction mixture was quenched with water and extracted with DCM (2×20 mL). The organic layer was dried over Na₂SO₄, filtered, concentrated, and purified by COMBIFLASH using 5% EtOAc-hexane as eluent to afford 750 mg of 4.

4 (750 mg), DCM (20 mL), 4 M HCl in dioxane (8 mL) were mixed at room temperature. After 16 hours the reaction volatiles are removed and product was purified by preparative HPLC and lyophilization to yield 230 mg of 1003.

### Example 7: Synthesis of Compound 1005 ((2R,3R,4R,5S)-2-(hydroxymethyl)-1-({4-[({3-methyl-5-[(morpholin-4-yl)methyl]phenyl}amino)methyl]phenyl}methyl)piperidine-3,4,5-triol)

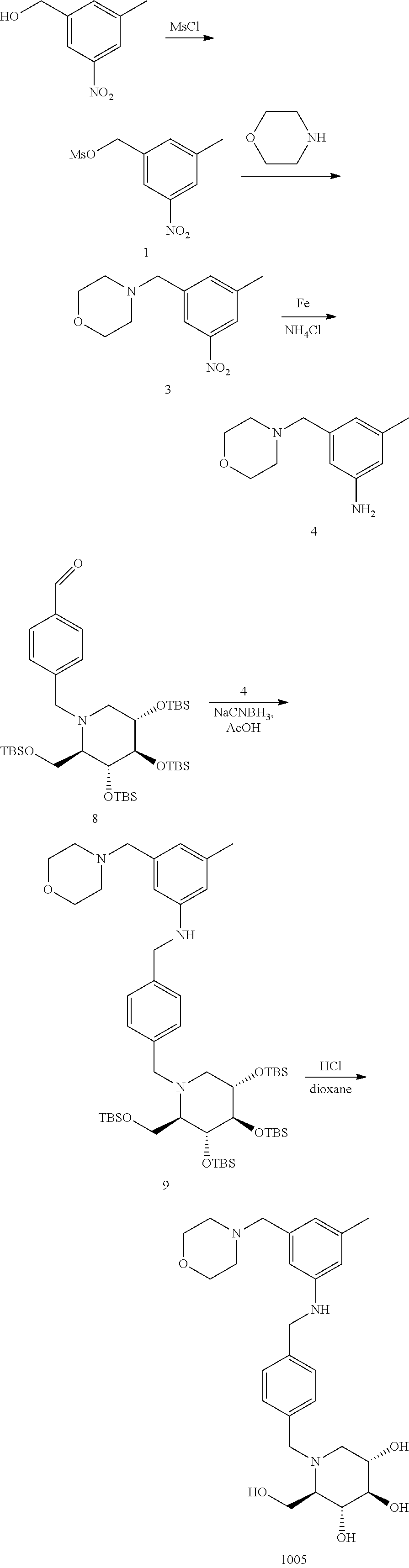

3-Methyl-5-hydroxymethyl-nitrobenzene (2.0 g), DCM (30 mL), Et₃N (3 eq), Ms-Cl (1.5 eq) were mixed. After 10 minutes the reaction was quenched with ice cold water and extracted with DCM (2×50 mL), the solvent was dried over anhydrous Na₂SO₄, filtered, and concentrated to give 2.4 g of 1.

1 (2.4 g), ACN (30 mL), morpholine (10 eq), K₂CO₃ (3 eq) were mixed at 80° C. After 8 hours the reaction was concentrated, residue was dissolved in ice cold water and extracted with EtOAc (2×50 mL). The solvent was dried over anhydrous Na₂SO₄, filtered, and concentrated to afford 2.5 g of 3 used as-is in the subsequent step.

3 (2.5 g), EtOH/water (2:1, 30 mL), NH₄Cl (12 eq), Fe (6 eq) were mixed at 80° C. After 16 hours the reaction was filtered through a bed of CELITE, washed with 10% MeOH/DCM. The filtrate was washed with water and dried over anhydrous Na₂SO₄, filtered, and concentrated to afford 1.4 g of 3-methyl-5-(4-morpholinyl-methyl)-benzenamine (4).

Int-2 (500 mg, Example 2), 4 (1 eq), DCM (15 mL), AcOH (0.2 mL), NaCNBH₃ (1.5 eq) were mixed at room temperature. After 16 hours the reaction was quenched with water and extracted with EtOAc (2×10 mL). The organic layer was dried over Na₂SO₄, filtered, concentrated, and purified by COMBIFLASH (35% EtOAc-hexane) to afford 300 mg of 9.

9 (300 mg), DCM (20 mL), 4.0 M HCl in dioxane (3 mL) were mixed at room temperature. After 16 hours the reaction was concentrated, triturated with DCM, and dried under vacuum to afford 160 mg of 1005 as HCl salt.

### Example 8: Synthesis of Compound 1006 ((2R,3R,4R,5S)-1-(6-{[3-cyclopropyl-5-(morpholin-4-yl)phenyl]amino}hexyl)-2-(hydroxymethyl)piperidine-3,4,5-triol)

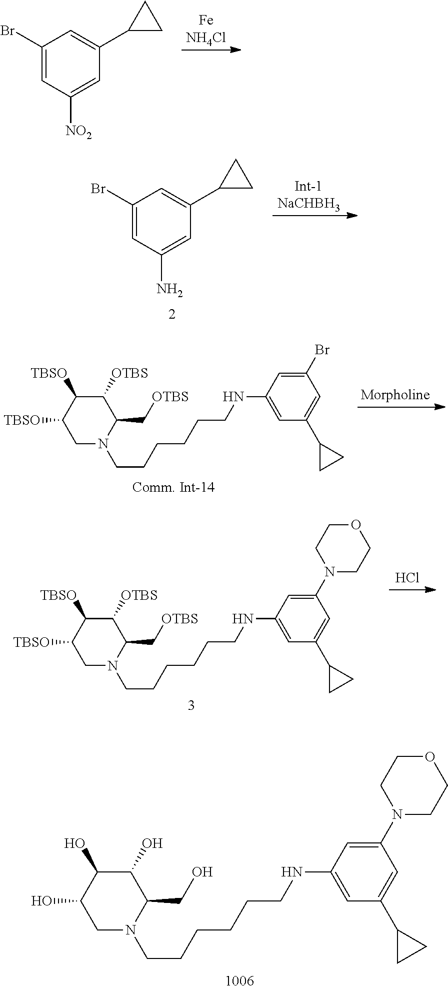 [SMILES: O=[N+]([O-])c1cc(Br)cc(C2CC2)c1 ; Nc1cc(Br)cc(C2CC2)c1 ; CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCCCCCNc1cc(Br)cc(C2CC2)c1 ; CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCCCCCNc1cc(C2CC2)cc(N2CCOCC2)c1 ; Cl ; OC[C@@H]1[C@@H](O)[C@H](O)[C@@H](O)CN1CCCCCCNc1cc(C2CC2)cc(N2CCOCC2)c1]

3-Bromo-5-cyclopropyl-nitrobenzene (3 g, Example 5), EtOH:H₂O (60 mL, 2:1), Fe (2 eq), NH₄Cl (4.0 eq) were added together at room temperature then temperature was increased to 90° C. After 4 hours the reaction was diluted with water and extracted with EtOAc. The organic layer was washed with water and dried over anhydrous Na₂SO₄, filtered, and concentrated to afford 2.5 g of 2 as colorless liquid.

2 (1.2 g), MeOH (80 mL), Int-1 prepared as in Example 1 (1.0 eq), AcOH (0.2 mL), and NaCNBH₃ (1.5 eq) were mixed at room temperature. After 16 hours the solvent was removed and the reaction mass diluted with water (100 mL) and extracted with DCM (2×100 mL). The organic layer was washed with water and dried over anhydrous Na₂SO₄, concentrated, and purified on silica [2% EtOAc-hexane] to afford 2.0 g of common Int-14.

Common Int-14 (100 mg), toluene (5 mL), Pd(OAc)₂ (0.2 eq), [(tBu)₃PH]BF₄ (0.2 eq), tBuONa (3.0 eq) was degassed with N₂ for 15 minutes, added morpholine (1.2 eq) and adjusted to 80° C. After 16 hours the reaction was diluted with EtOAc, washed with water, the organic layer was separated and dried over anhydrous Na₂SO₄, filtered, concentrated. Multiple combined batches were purified on silica [10% EtOAc in hexane] to afford 380 mg of 3 as thick colorless syrup.

3 (380 mg), DCM (5 mL), 4.0 M HCl in 1,4-dioxane (10 mL) mixed at 0° C. then warmed to room temperature and stirred 16 hours. Several similar batches were combined and purified to provide the HCl salt which was basified using aqueous (aq) NH₄HCO₃ and extracted with 5% MeOH/DCM then triturated with n-hexane to afford 100 mg of 1006.

### Example 9: Synthesis of Compound 1008 (2R,3R,4R,5S)-1-(6-{[3-cyclopropyl-5-(pyridazin-3-yl)phenyl]amino}hexyl)-2-(hydroxymethyl)piperidine-3,4,5-triol

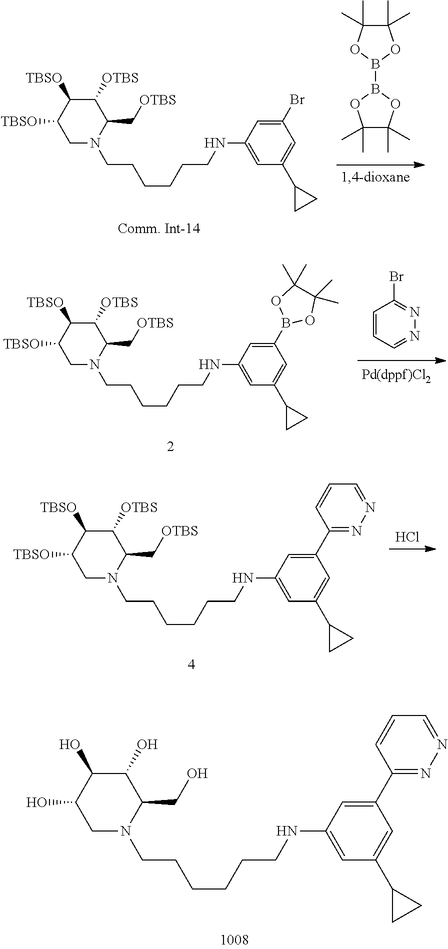

Common Int-14 (1 g), 1,4-dioxane (20 mL), bis-pinacolato-diboron (1.5 eq), KOAc (3.0 eq) were mixed and degassed for 15 minutes with N₂, added Pd(dppf)Cl₂ (0.1 eq) at room temperature then adjusted to 80° C. After 24 hours reaction was diluted with EtOAc and water. Separated the organic layer and dried over anhydrous Na₂SO₄, filtered, concentrated, and purified by COMBIFLASH eluted in 10% EtOAc/hexane to afford 1 g of 2.

2 (1 g), toluene:ethanol:water (1:1:1, 30 mL), 3-bromo-pyridazine (1.5 eq), Na₂CO₃ (3.0 eq) was degassed 15 minutes with N₂, Pd(dppf)Cl₂ (0.1 eq) added at room temperature then adjusted to 80° C. After 48 hours reaction mass was diluted with EtOAc and water. The organic layer was dried over anhydrous Na₂SO₄, filtered, concentrated, and purified by COMBIFLASH eluted with 10% EtOAc/hexane to afford 300 mg of 4.

4 (300 mg), DCM (9 mL), 4.0 M HCl in 1,4-dioxane (3.0 mL) were mixed at 0° C. then raised to room temperature. After 16 hours volatiles were removed and the product triturated with Et₂O (30 mL) and EtOAc (30 mL), filtered and dried to afford 20 mg 1008 as the HCl salt.

### Example 10: Synthesis of Compound 1009 ((2R,3R,4R,5S)-1-(6-{[3-cyclopropyl-5-(pyrimidin-2-yl)phenyl]amino}hexyl)-2-(hydroxymethyl)piperidine-3,4,5-triol)

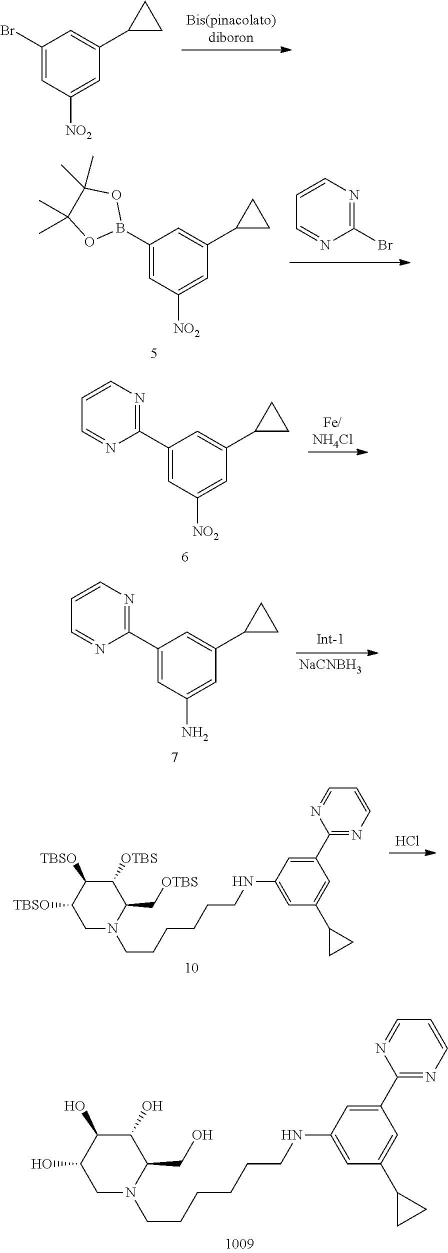

3-Bromo-5-cyclopropyl-nitrobenzene prepared as in Example 5 (10 g), 1,4-dioxane (200 mL), bis(pinacalato) diborane (1.5 eq), KOAc (3.0 eq), were degassed with N₂ for 20 minutes, added Pd(dppf)Cl₂ (0.1 eq) and increased to 100° C. After 6 hours the solvent was removed and the mass diluted with water and extracted with EtOAc (2×150 mL). The organic layer was dried over anhydrous Na₂SO₄ and concentrated to afford 11 g of 5.

5 (11 g), 2-bromo pyrimidine (1.5 eq), toluene:EtOH:water (1:1:1, 180 mL), Na₂CO₃ (3.0 eq) was degassed with N₂ for 20 minutes, added Pd(dppf)Cl₂ (0.1 eq) and heated to 100° C. After 16 hours the reaction was cooled to room temperature, volatiles removed, residue diluted with water and EtOAc and filtered through CELITE bed and filtrate was extracted with EtOAc (3×200 mL). The organic layer was dried over anhydrous Na₂SO₄, filtered, concentrated, then purified by COMBIFLASH [10% EtOAc:hexane] to afford 6.5 g of 6 as off white solid.

6 (39 g) EtOH:H₂O (4:1, 600 mL), Fe (5.0 eq), NH₄Cl (5.0 eq) were mixed at room temperature then heated to 100° C. After 1 hour volatiles were removed, residue was diluted with EtOAc and water and filtered through CELITE bed. The organic layer was separated, dried over anhydrous Na₂SO₄, filtered, and concentrated to afford 30 g of 7 as pale yellow syrup.

Common Int-1 (15.0 g), MeOH (300 mL), 7 (1.0 eq), AcOH (cat.) were mixed for 10 minutes at room temperature, NaCNBH₃ (1.5 eq) was added at 0° C. then raised to room temperature. After 16 hours volatiles were removed, residue was diluted with water (200 mL) and extracted with EtOAc (2×300 mL), the organic layer was washed with water, dried over anhydrous Na₂SO₄, filtered, concentrated, and purified by COMBIFLASH [5% EtOAc/hexane] to afford 7.8 g of 10.

10 (23 g), MeOH:DCM (1:1, 200 mL), 4.0 M HCl in 1,4-dioxane (230 mL) were added at 0° C. then warmed to room temperature. After 16 hours solvent was removed, the mass was diluted with water (100 mL) and washed with EtOAc (4×200 mL). The aqueous layer was neutralized with NaHCO₃ and extracted with 5% MeOH in EtOAc to afford 6.0 g of 1009.

### Example 11: Synthesis of Compound 1010 ((2R,3R,4R,5S)-1-{[4-({[3-cyclopropyl-5-(morpholin-4-yl)phenyl] amino}methyl)phenyl]methyl}-2-(hydroxymethyl)piperidine-3,4,5-triol)

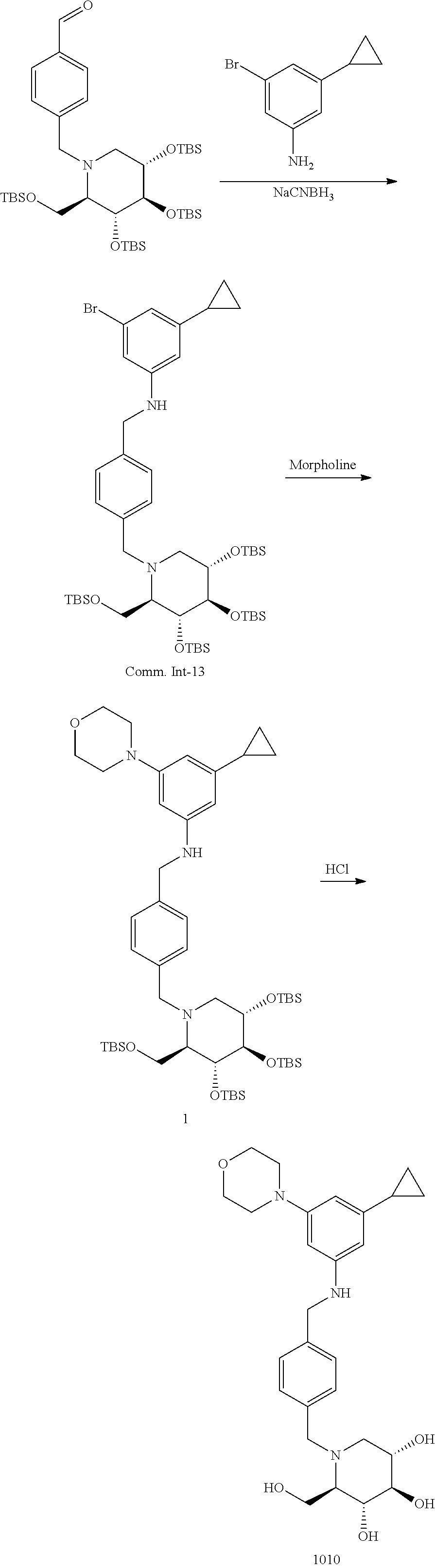

3-Bromo-5-cyclopropyl-aniline prepared as in Example 8 (1.2 g), MeOH (60 mL), Int-2 prepared as in Example 2 (0.7 eq), AcOH (0.2 mL), and NaCNBH₃ (1.5 eq) were mixed at room temperature. After 16 hours the solvent was removed and the reaction mass was diluted with water (100 mL) and extracted with DCM (2×100 mL). The organic layer was washed with water and dried over anhydrous Na₂SO₄ then concentrated and purified on silica with 2% EtOAc in hexane to afford 1.6 g of common Int-13.

Common Int-13 (1 g), toluene (20 mL), Pd₂(dba)₃ (0.05 eq), BINAP (0.1 eq), t-BuONa (2.5 eq) mixture was degassed with N₂ for 15 minutes, then morpholine (1.2 eq) added and temperature adjusted to 80° C. After 16 hours the reaction was diluted with EtOAc and washed with water. The organic layer was separated, dried over anhydrous Na₂SO₄, concentrated, and purified on silica [10% EtOAc-hexane] to obtain 400 mg of 1 as thick colorless syrup.

1 (400 mg), DCM (5 mL), 4.0 M HCl in 1,4-dioxane (3 mL) were mixed at 0° C. then temperature increased to room temperature. After 16 hours volatiles were concentrated. Product was washed with EtOAc (2 mL) and after lyophilization 190 mg of 1010 was obtained.

### Example 12: Synthesis of Compound 1013 ((2R,3R,4R,5S)-1-{[4-({[3-cyclopropyl-5-(pyrimidin-2-yl)phenyl]amino}methyl)phenyl]methyl}-2-(hydroxymethyl)piperidine-3,4,5-triol)

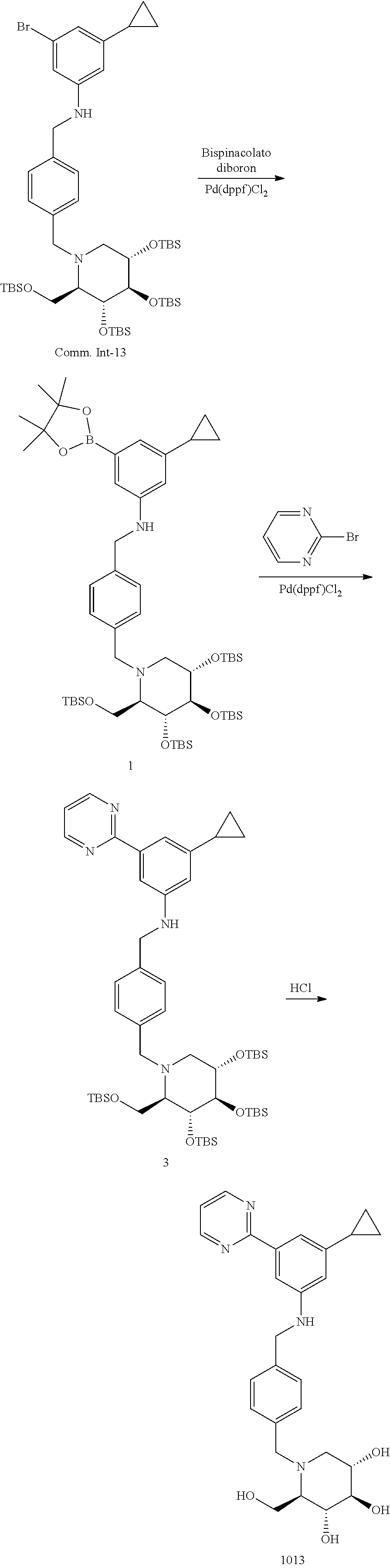

Common Int-13 (Example 11, 1.3 g), 1,4-dioxane (40 mL), bis(pinacalato) diboron (1.5 eq), KOAc (3.0 eq) was degassed with N₂ for 15 minutes, added Pd(dppf)Cl₂ (0.2 eq) and temperature increased to 100° C. After 16 hours the reaction was diluted with water (50 mL) and extracted with EtOAc (2×50 mL). The organic layer was washed with water, dried over anhydrous Na₂SO₄, concentrated to afford 1 (1.3 g).

1 (1.3 g), toluene:EtOH:water (45 mL), 2-bromopyrimidine (1.5 eq), Na₂CO₃ (3.0 eq) degassed with N₂ for 15 minutes, added Pd(dppf)Cl₂ (0.2 eq) and heated to 90° C. After 16 hours the reaction was diluted with water (20 mL), extracted with EtOAc (2×20 mL), the organic layer was washed with water, dried over anhydrous Na₂SO₄, filtered, concentrated, and purified by COMBIFLASH [5% EtOAc-hexane] to afford 750 mg of 3.

3 (750 mg), DCM (25 mL) were mixed at 0° C. then added 4.0 M HCl in 1,4-dioxane (5 mL) and temperature increased to room temperature. After 16 hours the volatiles were removed and product washed with EtOAc then Et₂O. After lyophilisation 295 mg of 1013 was obtained.

### Example 13: Synthesis of Compounds derived from Common Int.-1

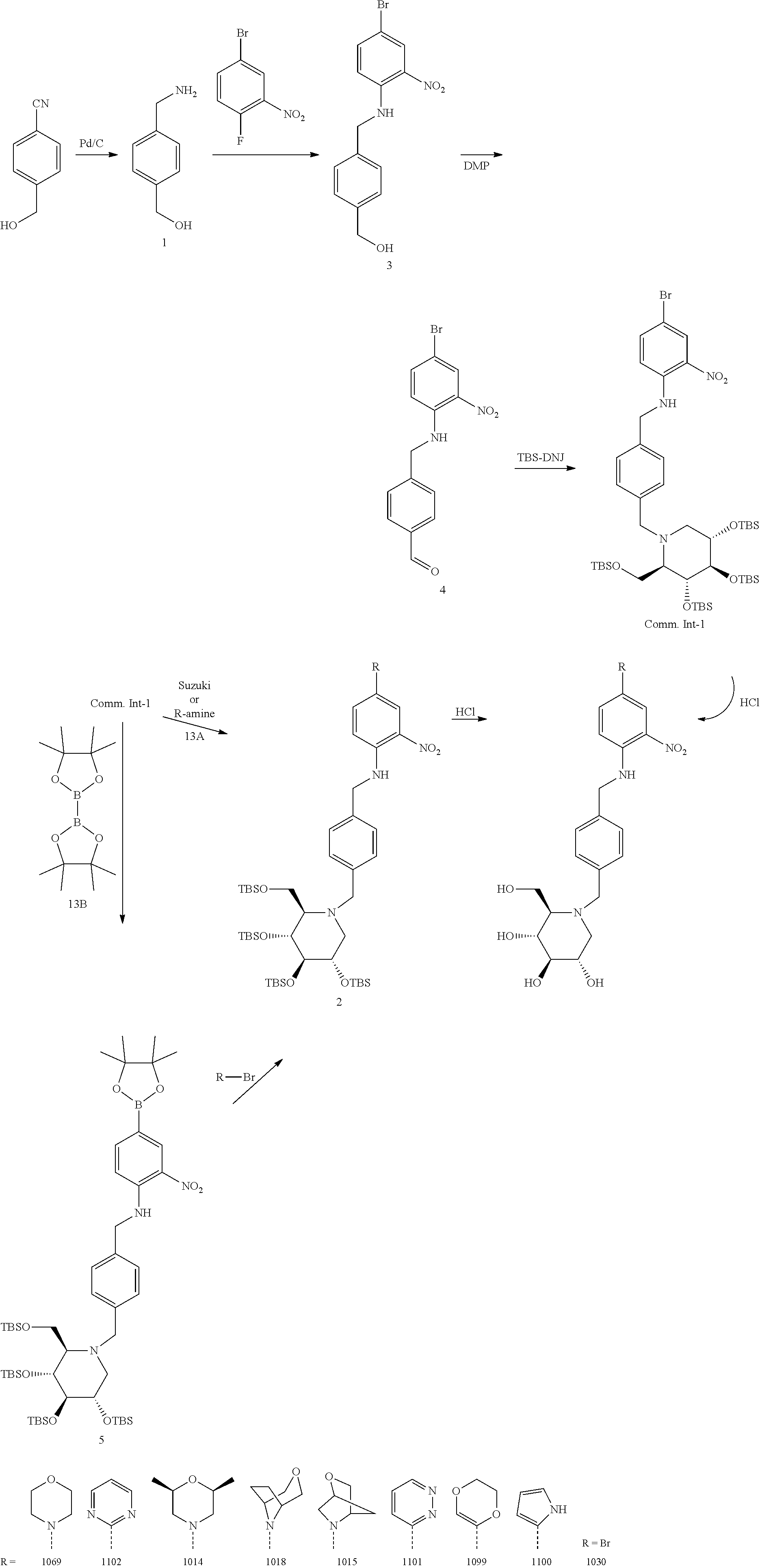

4-Cyano-benzylalcohol (50 g) in MeOH (1250 mL) with 10% Pd/C (50 g, 50% wet) was autoclaved under H₂ at room temperature and 200 psi for 16 hours. The reaction was filtered through a pad of CELITE, washed with MeOH, the filtrate was concentrated and triturated with Et₂O (500 mL), filtered and dried to afford 40 g of 1 as white solid.

1 (10 g), 1,4-dioxane (200 mL), 1-fluoro-4-bromo-2-nitrobenzene (1.2 eq), Et₃N (5.0 eq) mixed at room temperature then increased to 100° C. After 6 hours volatiles were removed, residue was dissolved in water and extracted with EtOAc, the organic layer was dried over anhydrous Na₂SO₄, concentrated and purified on silica [30% EtOAc-hexane] to afford 6 g of 3.

3 (6 g), DCM (120 mL), Dess-Martin Periodinane (DMP), (1.5 eq), mixed at 0° C. then increased to room temperature. After 1 hour reaction was diluted with water, extracted with DCM, the organic layer was dried over anhydrous Na₂SO₄ then concentrated to afford 5 g of 4.

4 (5 g), MeOH (50 mL), DCM (50 mL), TBS-DNJ (Example 1, 0.8 eq), AcOH (cat.), NaCNBH₃ (1.5 eq) were mixed at room temperature. After 16 hours the solvent was removed. The residue was dissolved with water and extracted with EtOAc (2×100 mL). The organic layers were washed with brine, dried over anhydrous Na₂SO₄, filtered, concentrated, and purified on silica [5% EtOAc in hexane] to afford 6.0 g of Common Int-1.

For Schemes 13B, in one example Common Int-1 (5.0 g), 1,4-dioxane (100 mL), bis(pinacolato) diboron (1.5 eq), KOAc (3.0 eq) were degassed 15 minutes with N₂, added Pd(dppf)Cl₂ (0.1 eq) then increased to 100° C. After 16 hours reaction mass was diluted with water, extracted with EtOAc (2×150 mL), organic layers were washed with brine solution, dried over anhydrous Na₂SO₄, filtered, and concentrated to yield boronate 5.

Preparation of 1014 (2S,3S,4S,5R)-1-({4-[({4-[(2R,6S)-2,6-dimethylmorpholin-4-yl]-2-nitrophenyl}amino)methyl]phenyl}methyl)-2-(hydroxymethyl)piperidine-3,4,5-triol (13A): Common Int. 1 (300 mg) in toluene (5 mL) was degassed 30 minutes with N₂ then 2S,6R-dimethylmorpholine (6 eq) then tBuONa (3.0 eq), Pd(OAc)₂ (0.4 eq), [(t-Bu)₃PH]BF₄ (0.4 eq) were added at room temperature then heated to 100° C. After 4 hours the reaction was cooled to room temperature, then added water and extracted with EtOAc (20 mL). The organic layer was separated, dried over Na₂SO₄, filtered, concentrated, and purified on silica using eluting with 5% EtOAc:hexane to obtain 280 mg of 2.

2 (300 mg) was mixed in MeOH/DCM (1:1) (15 mL) at 0° C. then 4 M HCl in dioxane (3 mL) was added at 0° C. then warmed to room temperature. After 16 hours volatiles were removed and product triturated with DCM and filtered to afford 144 mg of 1014 as HCl salt.

Preparation of 1015 (2S,3S,4S,5R)-2-(hydroxymethyl)-1-[(4-{[(2-nitro-4-{2-oxa-5-azabicyclo[2.2.1]heptan-5-yl}phenyl)amino]methyl}phenyl)methyl]piperidine-3,4,5-triol (13A): Common Int. 1 (400 mg) in toluene (12 mL) was degassed for 30 minutes with N₂, then 2-oxa-5-azabicyclo[2.2.1]heptane hydrochloride (1.5 eq) was added then tBuONa (5.0 eq), Pd(OAc)₂ (0.4 eq), [(t-Bu)₃PH]BF₄ (0.2 eq) at room temperature then heated to 100° C. After 2 hours the reaction was cooled to room temperature and added water then extracted with EtOAc (30 mL). The organic layer was separated, dried over Na₂SO₄, filtered, concentrated, and purified on silica [5-20% EtOAc/hexane] to afford 2 (200 mg).

2 (200 mg) was mixed in DCM (8 mL) at 0° C., then 4.0 M HCl in dioxane (2 mL) was added at room temperature. After 16 hours reaction volatiles were removed, and the residue washed with EtOAc (3×30 mL), DCM (2×20 mL) and Et₂O (3×30 mL), filtered, and dried to afford 55 mg 1015 as the HCl salt.

Preparation of 1018 (2S,3S,4S,5R)-2-(hydroxymethyl)-1-({4-[({2-nitro-4-[(1R,5S)-3-oxa-8-azabicyclo[3.2.1]octan-8-yl]phenyl}amino)methyl]phenyl}methyl) piperidine-3,4,5-triol (13A): Common Int. 1 (100 mg) in toluene (10 mL) was degassed for 30 minutes with N₂, 3-oxa-8-azabicyclo[3.2.1]octane hydrochloride (1.5 eq) was added then tBuONa (6 eq), Pd (OAc)₂ (0.4 eq), [(t-Bu)₃PH]BF₄ (0.4 eq) at room temperature then heated to 100° C. After 4 hours the reaction mixture was cooled to room temperature, added water and extracted with EtOAc (15 mL). The organic layer was separated, dried over Na₂SO₄, filtered, and concentrated. Several combined batches were purified on silica eluting with 5% EtOAc-hexane to obtain 190 mg of 2.

2 (190 mg) and MeOH/DCM (1:1, 8 mL) were mixed at 0° C. then added 4.0 M HCl in dioxane (1.9 mL) and raised to room temperature. After 16 hours reaction volatiles were removed. The material was triturated with DCM, filtered, dissolved in water and washed with Et₂O. The aqueous layer was basified using saturated aqueous NH₄HCO₃ solution. The precipitated material was filtered and dried to afford 44 mg of 1018.

Preparation of 1030 (2R,3R,4R,5S)-1-[(4-{[(4-bromo-2-nitrophenyl)amino]methyl}phenyl)methyl]-2-(hydroxymethyl)piperidine-3,4,5-triol and 1099 (2R,3R,4R,5S)-1-{[4-({[4-(5,6-dihydro-1,4-dioxin-2-yl)-2-nitrophenyl]amino}methyl)phenyl]methyl}-2-(hydroxymethyl)piperidine-3,4,5-triol (13A): For this target it proved more convenient to carry out the deprotection prior to the coupling. Deprotection was accomplished by mixing Common Int-1 (4.0 g), MeOH (60 mL), DCM (5 mL), 4.0 MHCl in 1,4-dioxane (40 mL) at 0° C. then increasing to room temperature. After 16 hours solvent was removed and reaction mass was basified with NaHCO₃. The reaction was diluted with water (50 mL) and extracted with EtOAc (3×100 mL). The combined organic layers were dried over anhydrous Na₂SO₄, filtered, concentrated to afford 2.0 g of 1030.

1030 (2 g), 1,4-dioxane:H₂O (3:1, 120 mL), Cs₂CO₃ (3.0 eq), 2-(5,6-dihydro-1,4-dioxin-2-yl)-boronate (1.5 eq), degassed for 15 minutes with N₂, Pd(dppf)Cl₂ (0.1 eq) added at room temperature then heated to 80° C. After 16 hours reaction was diluted with water (50 mL), extracted with EtOAc (3×75 mL). Combined organic layers were dried over anhydrous Na₂SO₄, filtered, concentrated, and preparative HPLC purification gave 200 mg of 1099.

Preparation of 1069 (2S,3S,4S,5R)-2-(hydroxymethyl)-1-{[4-({[4-(morpholin-4-yl)-2-nitrophenyl]amino}methyl)phenyl]methyl}piperidine-3,4,5-triol (13A): Common Int-1 (5 g), morpholine (3 eq), dioxane (200 mL), Cs₂CO₃ (2 eq), Pd(dba)₃ (0.2 eq), xantphos (0.2 eq) mixed, degassed 15 minutes with N₂, and heated 8 hours at 90° C. Reaction was cooled to room temperature, quenched with water, extracted with EtOAc (200 mL), organic layer dried over Na₂SO₄, filtered, concentrated, and purified on silica [5% EtOAc-hexane] to give 1.5 g of 2.

Mixed 2 (1.2 g), MeOH:DCM (1:1, 30 mL) at 0° C., 4.0 MHCl in 1,4-dioxane (15 mL) added then increased to room temperature. After 16 hours the reaction was concentrated, triturated with DCM (100 mL), and the solids were filtered and dried. Blending with a separate small batch afforded 487 mg 1069 as red solid.

Preparation of 1100 (2R,3R,4R,5S)-2-(hydroxymethyl)-1-{[4-({[2-nitro-4-(1H-pyrrol-2-yl)phenyl]amino}methyl)phenyl]methyl}piperidine-3,4,5-triol (13A): Common Int-1 (5.0 g), toluene:ethanol:water (1:1:1, 120 mL), 1-Boc-pyrrole-2-boronate (1.0 eq), Na₂CO₃ (3.0 eq), degassed for 15 minutes with N₂, Pd(dppf)Cl₂ (0.1 eq) addition at room temperature then increased to 80° C. After 16 hours reaction volatiles were removed and residue diluted with EtOAc and water. Separated the organic layer and dried over anhydrous Na₂SO₄, concentrated, and purified on silica in 5-10% EtOAc/hexane to afford 3.8 g of 2.

2 (3.8 g), MeOH:DCM (4:1, 76 mL) mixed at 0° C. then 4.0M HCl in dioxane (38 mL) added at 0° C. then reacted 16 hours at room temperature. Volatiles were removed, and residue washed with Et₂O. The solid was dissolved in water (20 mL), neutralized with saturated NaHCO₃ at 0° C. and the precipitate filtered to afford 1.5 g of dark solid, 100 mg of which was purified on silica in 5-10% MeOH/DCM/aq. NH₃ (0.1%) to afford 35 mg of 1100. The remaining dark solid was purified by preparative HPLC to afford 120 mg of 1100.

Preparation of 1101 (2R,3R,4R,5S)-2-(hydroxymethyl)-1-{[4-({[2-nitro-4-(pyridazin-3-yl)phenyl]amino}methyl)phenyl]methyl}piperidine-3,4,5-triol (13B): Boronate 5 (6.0 g), toluene:ethanol:water (1:1:1, 120 mL), 3-bromopyridazine (1.2 eq), Na₂CO₃ (3.0 eq), degassed 15 minutes with N₂, then Pd(dppf)Cl₂ (0.1 eq) addition at room temperature then increased to 80° C. After 6 hours solvent was removed, reaction mass was diluted with water and extracted with EtOAc (2×100 mL). The combined organic layers were washed with brine solution, dried over anhydrous Na₂SO₄, filtered, concentrated, and purified on silica [eluent 15% EtOAc/hexane] to afford 3.0 g of 4.

4 (3.0 g), MeOH (50 mL), DCM (10 mL) mixed at 0° C., 4.0 MHCl in 1,4-dioxane (20 mL) added at 0° C. then increased to room temperature. After 16 hours solvent was removed. The reaction mass was washed with 2×50 mL EtOAc, diluted with water and basified with saturated aq. NaHCO₃. Precipitate was filtered and washed with n-hexane to afford 1.3 g of 1101.

Preparation of 1102 (2R,3R,4R,5S)-2-(hydroxymethyl)-1-{[4-({[2-nitro-4-(pyrimidin-2-yl)phenyl]amino}methyl)phenyl]methyl}piperidine-3,4,5-triol (13B): Boronate 5 (6.0 g), toluene:ethanol:water (1:1:1, 120 mL), 2-bromopyrimidine (1.2 eq), Na₂CO₃ (3.0 eq) were degassed 15 minutes with N₂, Pd(dppf)Cl₂ (0.1 eq) added at room temperature then 80° C. for 6 hours. The solvent was removed. The reaction mass was diluted with water and extracted with EtOAc (2×100 mL). The combined organic layers were washed with brine solution, dried over anhydrous Na₂SO₄, filtered, concentrated, and purified on silica [15% EtOAc in hexane] to afford 3.0 g of 2.

2 (3.0 g), MeOH (50 mL), DCM (10 mL) mixed at 0° C., 4.0 MHCl in 1,4-dioxane (20 mL) added at 0° C. then room temperature for 16 hours. The solvent was removed, reaction mass was washed with 2×50 mL EtOAc, diluted in water, and basified with saturated aq. NaHCO₃. The precipitate was filtered and washed with n-hexane to afford 1.2 g of 1102.

### Example 14: Synthesis of Compounds Derived from Common Int.-4

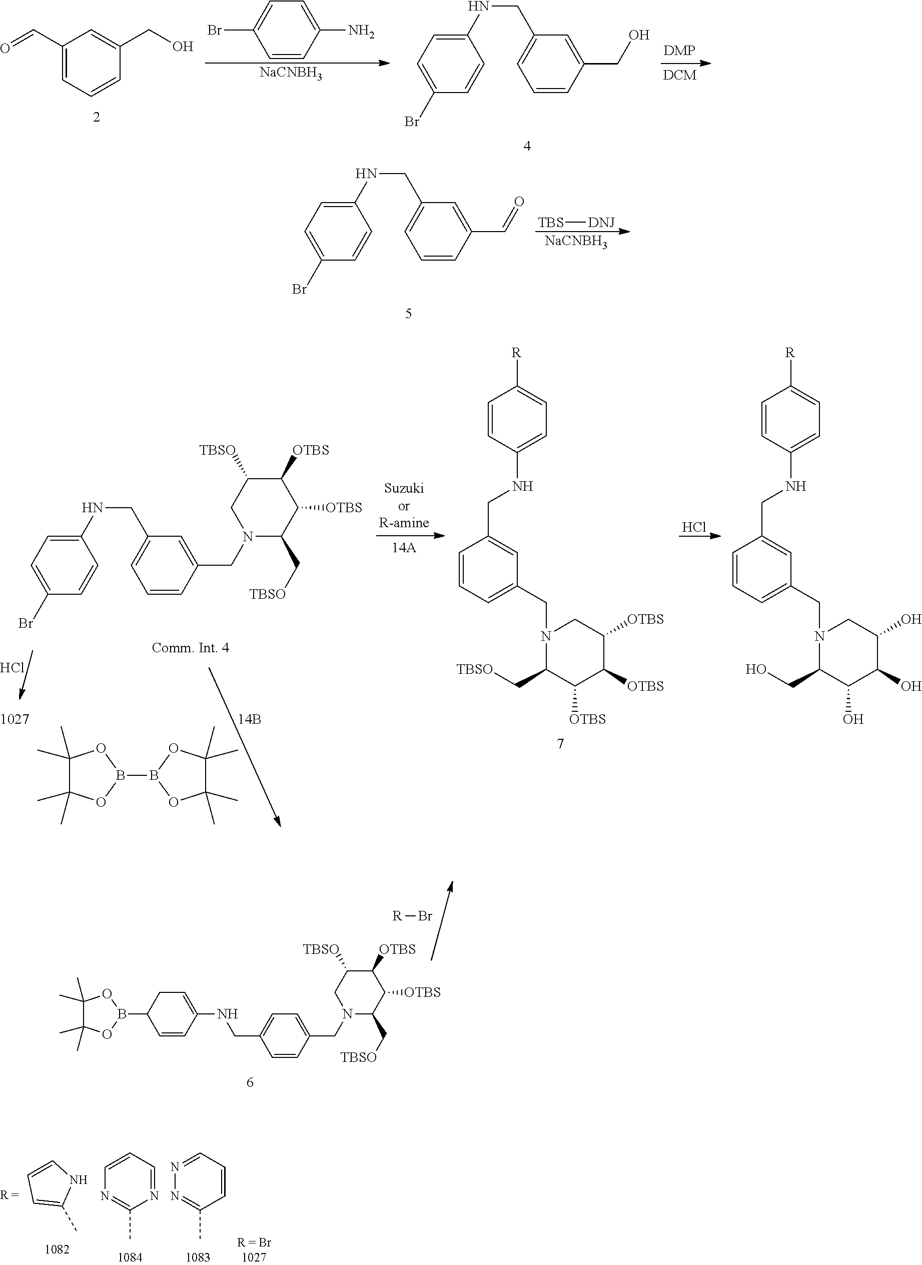

2 prepared as in Example 3 (50 g), MeOH (1 L), 4-bromoaniline (1.0 eq) mixed at 0° C. then AcOH (cat.), NaCNBH₃ (1.5 eq) addition at 0° C., then room temperature for 16 hours. The reaction was concentrated, the residue dissolved in water and extracted with EtOAc (2×400 mL). The organic layer was washed with water then dried over anhydrous Na₂SO₄, concentrated, and purified on silica in 10-20% EtOAc/hexane to afford 35 g of 4.

4 (19 g), DCM (1.9 L) mixed at 0° C., DMP (1 eq) added at 0° C., then stirred for 15 minutes. The reaction was diluted with water and extracted with DCM. The organic layer was dried over anhydrous Na₂SO₄ concentrated to afford 19 g of 5.

5 was alternatively prepared using Swern oxidation conditions. (COCl)₂ (3.0 eq), DCM (40 mL) were cooled to −78° C., DMSO (4 eq) mixed for 30 minutes, 4 (5 g) mixed for 2 hours TEA (8 eq) added and temperature increased to room temperature over 30 minutes. After 3 hours the reaction was diluted with water and extracted with DCM (2×200 mL). Solvent was dried over Na₂SO₄, filtered, and 5 in the solvent was directly used in next step.

5 (19 g), MeOH (600 mL), TBS-DNJ (1.0 eq), mixed at 0° C., AcOH (6 mL) and NaCNBH₃ (1.5 eq) addition at 0° C., then increased to room temperature. After 16 hours reaction was concentrated. The residue was dissolved in water and extracted with EtOAc (2×300 mL). The organic layer was washed with water, dried over anhydrous Na₂SO₄, concentrated, and purified on silica in 2% EtOAc/hexane to afford 20 g of common Int.-4.

For schemes 14B, Common Int. 4 (6.0 g), bis(pinacolato) diboron (2.2 eq), KOAc (3.0 eq), degassed 15 minutes under N₂, added Pd(dppf)Cl₂ (0.1 eq), 1,4-dioxane (120 mL) mixed, and raised to 90° C. After 16 hours solvents were removed and the reaction mass was diluted with EtOAc (150 mL), washed with water (2×100 mL), brine and dried over anhydrous Na₂SO₄. The organic layer was concentrated to afford 6.0 g of boronate 6.

Preparation of 1027 (2R,3R,4R,5S)-1-[(3-{[(4-bromophenyl)amino]methyl}phenyl)methyl]-2-(hydroxymethyl)piperidine-3,4,5-triol: Common Int.-4 (4 g), MeOH (30 mL), DCM (30 mL), 4 M HCl in dioxane (40 mL), added at 0° C. then warmed to room temperature. After 8 hours solvent was removed, residue dissolved in water, basified with saturated NaHCO₃ and extracted with EtOAc. The organic layer was dried over anhydrous Na₂SO₄ then concentrated to afford 1.7 g of 1027 as off-white solid.

Preparation of 1082 (2R,3R,4R,5S)-2-(hydroxymethyl)-1-{[3-({[4-(1H-pyrrol-2-yl)phenyl]amino}methyl)phenyl]methyl}piperidine-3,4,5-triol (14A): Common Int-4 (5 g), toluene:EtOH:water (100 mL, 1:1:1), 1-Boc-pyrrole-2-boronate (1 eq), Na₂CO₃ (3.0 eq), degassed 15 minutes under N₂, added Pd(dppf)Cl₂ (0.1 eq) mixed, and heated to 100° C. After 16 hours the reaction was cooled to room temperature, diluted with water and extracted with EtOAc (2×500 mL). The organic layer was dried over anhydrous Na₂SO₄, filtered, concentrated, and purified on silica with 1% EtOAc-hexane to afford 3.0 g of 7.

7 (2.0 g), DCM (100 mL), 4.0 M HCl in 1,4-dioxane (40 mL) added at room temperature then stirred for 8 hours. The reaction volatiles were removed, residue dissolved in water and basified with solid NH₄HCO₃. Volatiles were removed and after preparative HPLC purification and lyophilization 190 mg of 1082 was obtained.

Preparation of 1083 (2R,3R,4R,5S)-2-(hydroxymethyl)-1-{[3-({[4-(pyridazin-3-yl)phenyl]amino}methyl)phenyl]methyl}piperidine-3,4,5-triol (14B): Mixed boronate 6 (3.0 g), 3-bromo-pyridazine (1.1 eq), Na₂CO₃ (3.0 eq), in toluene:EtOH:water (1:1:1, 60 mL), degassed 15 minutes under N₂, added Pd(dppf)Cl₂ (0.1 eq) raised to 90° C. After 16 hours reaction volatiles are removed, residue diluted with water (60 mL) and extracted with EtOAc (2×50 mL). The combined organic layers were dried over anhydrous Na₂SO₄ filtered, concentrated, purification on silica [25% EtOAc in hexane] afforded 1.2 g of 7.

7 (2.3 g), DCM (10 mL), MeOH (10 mL) mixed at 0° C. the 4.0 MHCl in 1,4-dioxane (10 mL) added and raised to room temperature. After 16 hours volatile solvents were removed, and triturated with Et₂O, EtOAc followed by ACN to afford 1.06 g of 1083 as HCl salt.

Preparation of 1084 (2R,3R,4R,5S)-2-(hydroxymethyl)-1-{[3-({[4-(pyrimidin-2-yl) phenyl]amino}methyl)phenyl]methyl}piperidine-3,4,5-triol (14B): Common Int-4 (4.4 g), 2-bromopyrimidine (2.0 eq), K₂CO₃ (3.0 eq), 1,4-dioxane:water (3:1, 120 mL) mixed, degassed 15 minutes under N₂, added Pd(PPh₃)₄ (0.1 eq), heated to 100° C. After 16 hours removed reaction volatiles and residue was diluted with water (50 mL) and extracted with EtOAc (2×50 mL). Combined organic layers were dried over anhydrous Na₂SO₄, filtered, concentrated, and purified on silica [4% EtOAc:hexane] to afford 1.5 g of 7.

7 (1.5 g), DCM (15 mL), MeOH (15 mL) mixed at 0° C. then 4.0 MHCl in 1,4-dioxane (7 mL) added and increased to room temperature. After 16 hours volatile solvents were removed, and residue triturated with Et₂O, EtOAc, and ACN. Combined batches lyophilized to afford 740 mg 1084 as HCl salt.

### Example 15: Synthesis of Compounds Derived from Common Int.-3

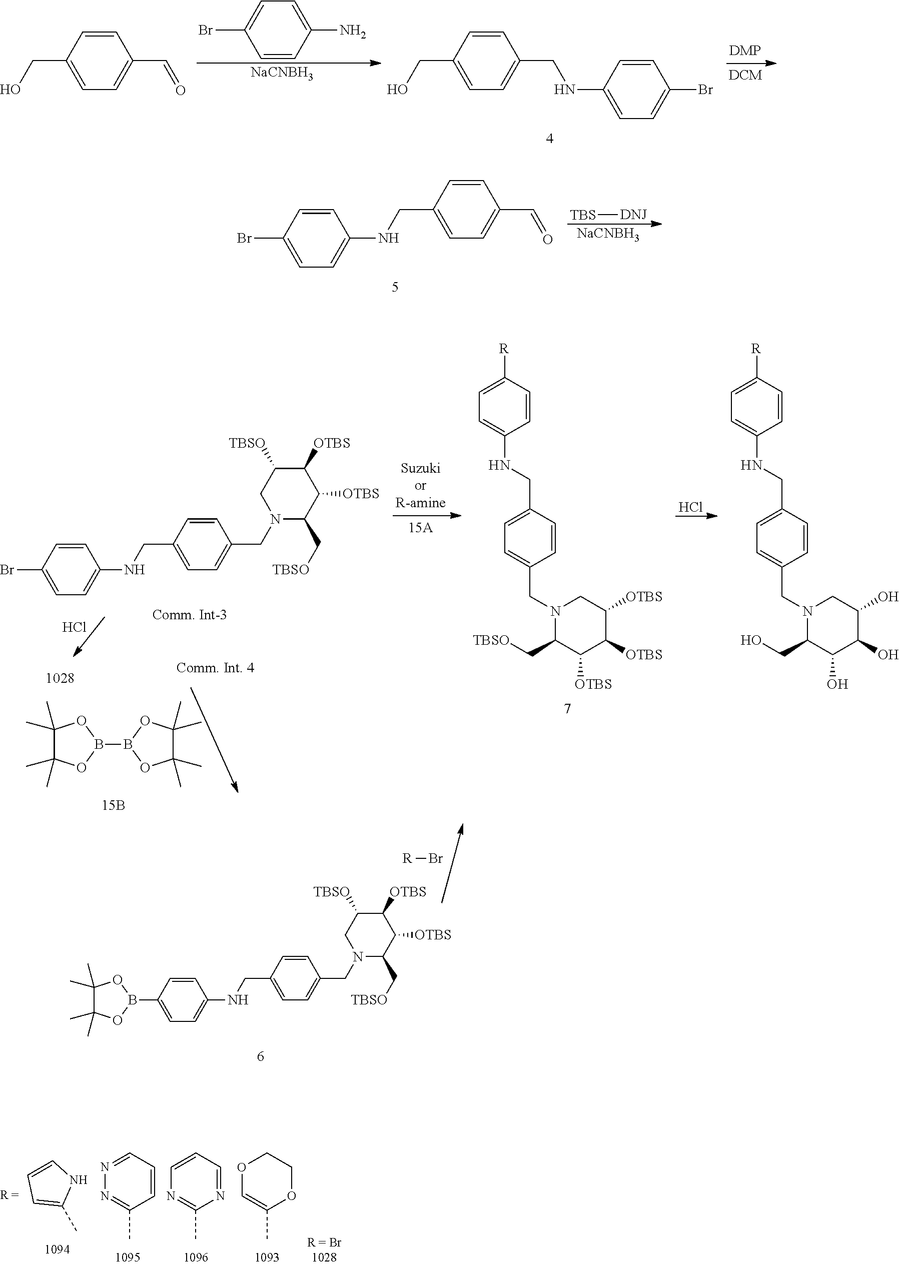

4-(Hydroxymethyl)-benzaldehyde (29 g), MeOH (600 mL), 4-bromoaniline (1.0 eq) were mixed at 0° C. then AcOH (cat.), NaCNBH₃ (1.5 eq) addition then increased to room temperature. After 16 hours reaction was diluted with water and extracted with DCM (2×500 mL). Organic layer was washed with water and dried over anhydrous Na₂SO₄, concentrated, and purified on silica [10-20% EtOAc/hexane] to afford 30 g of 4.

4 (30 g), DCM (3.0 L) mixed at 0° C., DMP (1.5 eq) added at 0° C. After 15 minutes the reaction mass was filtered through pad of CELITE and filtrate DCM was washed with water, dried over anhydrous Na₂SO₄, and concentrated to afford 30 g of 5.

5 (30 g), MeOH (600 mL), TBS-DNJ (1.0 eq), AcOH (cat.), NaCNBH₃ (1.5 eq) mixed at room temperature for 16 hours. Reaction volatiles were removed, residue diluted with EtOAc and water. Separated the organic layer and dried over anhydrous Na₂SO₄, concentrated, and purified on silica [1-2% EtOAc in hexane] to afford 28 g of common Int.-3.

For Scheme 15B, Common Int-3 (5.0 g), 1,4-dioxane (120 mL), bis(pinacolato) diboron (1.5 eq), KOAc (3.0 eq) were degassed for 15 minutes with N₂, Pd(dppf)Cl₂ (0.1 eq) added then heated to 100° C. After 16 hours reaction mass was diluted with water and extracted with EtOAc (2×15 mL). The combined organic layers were washed with brine solution, dried over anhydrous Na₂SO₄, filtered, concentrated to afford 6.0 g of boronate 6.

Preparation of 1094 (2R,3R,4R,5S)-2-(hydroxymethyl)-1-{[4-({[4-(1H-pyrrol-2-yl) phenyl]amino}methyl)phenyl]methyl}piperidine-3,4,5-triol (15A): CommonInt-3 (5.0 g), toluene:ethanol:water (1:1:1, 120 mL), 1-Boc-pyrrole-2-boronate (1.0 eq), Na₂CO₃ (3.0 eq), degassed with N₂ 15 minutes, added Pd(dppf)Cl₂ (0.1 eq) at room temperature then heated to 80° C. After 16 hours reaction volatiles were removed. The residue was diluted with EtOAc and water. Separated the organic layer and dried over anhydrous Na₂SO₄, concentrated, and purified on silica [2% EtOAc in hexane] to afford 2.5 g of 7.

7 (2.3 g), MeOH:DCM (1:1, 60 mL), mixed at 0° C., added 4.0 M HCl in 1,4-dioxane (20 mL) at 0° C. then room temperature for 24 hours. Volatiles were removed, residue was triturated with EtOAc then Et₂O. Preparative HPLC purification gave 140 mg of 1094 as off white solid.

Preparation of 1096 (2R,3R,4R,5S)-2-(hydroxymethyl)-1-{[4-({[4-(pyrimidin-2-yl) phenyl]amino}methyl)phenyl]methyl}piperidine-3,4,5-triol (15B): 2-bromo-pyrimidine (1.0 eq), 6 (4.5 g), toluene:ethanol:water (1:1:1, 120 mL), Na₂CO₃ (3.0 eq), degassed for 15 minutes with N₂, Pd(dppf)Cl₂ (0.1 eq) addition at room temperature then 80° C. for 16 hours. Reaction volatiles were removed and residue diluted with EtOAc and water. Separated the organic layer and dried over anhydrous Na₂SO₄, concentrated, and purified on silica [10% EtOAc in hexane] to afford 1.2 g of 7.

7 (1.2 g), DCM (12 mL), 4.0 MHCl in 1,4-dioxane (6.0 mL) addition at 0° C. then room temperature for 16 hours. Volatiles were removed, and triturated with Et₂O, EtOAc, DCM (2×10 mL each) to afford 400 mg of yellow solid. The solid was dissolved in minimum amount of water and neutralized with aq. NH₄HCO₃ dropwise which precipitated solid which was filtered and again triturated to afford 200 mg of 1096 as off white solid.

Preparation of 1095 (2R,3R,4R,5S)-2-(hydroxymethyl)-1-{[4-({[4-(pyridazin-3-yl) phenyl]amino}methyl)phenyl]methyl}piperidine-3,4,5-triol (15B): 6 (3.0 g), toluene:ethanol:water (1:1:1, 90 mL), 2-bromopyridizine (1.0 eq), Na₂CO₃ (3.0 eq), degassed for 15 minutes with N₂, Pd(dppf)Cl₂ (0.1 eq) added at room temperature then heated to 80° C. After 16 hours reaction volatiles were removed and residue diluted with EtOAc and water. Separated the organic layer and dried over anhydrous Na₂SO₄ and concentrated. This material was purified on silica [10% EtOAc in hexane] to afford 1.2 g of 7.

7 (1.2 g), MeOH:DCM (2:1, 18 mL), 4.0 MHCl in 1,4-dioxane (6.0 mL) addition at 0° C. then reacted at room temperature for 16 hours. Volatiles were removed, and residue washed with Et₂O to afford solid which was dissolved in water (5 mL), neutralized with saturated NaHCO₃ at 0° C., resulting solid was filtered and washed with n-hexane to afford 200 mg of 1095 as off white solid. An additional 500 mg of less pure material was not further purified.

Preparation of 1028 (2R,3R,4R,5S)-1-[(4-{[(4-bromophenyl)amino]methyl}phenyl)methyl]-2-(hydroxymethyl)piperidine-3,4,5-triol and 1093 (2R,3R,4R,5S)-1-{[4-({[4-(5,6-dihydro-1,4-dioxin-2-yl)phenyl]amino}methyl)phenyl]methyl}-2-(hydroxymethyl)piperidine-3,4,5-triol (15A): For this target it proved more convenient to carry out deprotection prior to coupling. Mixed common Int. 3 (3.0 g) in MeOH (45 mL) and DCM (1 mL), added 4.0 M HCl in 1,4-dioxane (30 mL) at 0° C. then reacted at room temperature for 16 hours. Solvent was removed and reaction mass was basified with NaHCO₃. The mix was diluted with water (30 mL) and extracted with EtOAc (3×50 mL). The combined organic layers were dried over anhydrous Na₂SO₄, filtered, concentrated, to afford 1.2 g of 1028.

1028 (500 mg), 1,4-dioxane:H₂O (3:1, 20 mL), 2-(5,6-dihydro-1,4-dioxin-2-yl)-boronate (2.0 eq), Cs₂CO₃ (4.0 eq), degassed for 15 minutes with N₂, Pd(dppf)Cl₂ (0.15 eq) added at room temperature then heated at 100° C. for 8 hours. Reaction was diluted with water (20 mL) and extracted with EtOAc (3×50 mL). The combined organic layers were dried over anhydrous Na₂SO₄ filtered, concentrated, and triturated with Et₂O, filtered and dried then washed with water and filtered to afford 150 mg of 1093 as off white solid.

### Example 16: Synthesis of Compounds 1086 ((2R,3R,4R,5S)-2-(hydroxymethyl)-1-([{3-[([{2-nitro-4-[(1,2-oxazolidi-2-yl)methyl]phenyl}amino)methyl]phenyl}methyl)piperidine-3,4,5-triol) and 1020 ((2R,3R,4R,5S)-2-(hydroxymethyl)-1-[(3-{[methyl([{2-nitro-4-[(1,2-oxazolidin-2-yl)methyl]phenyl})amino]methyl}phenyl)methyl]piperidine-3,4,5-triol)

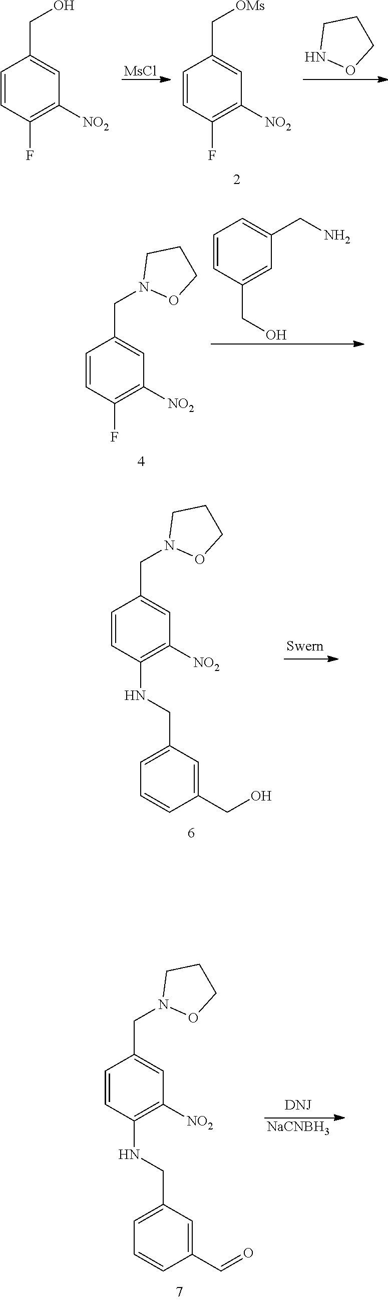

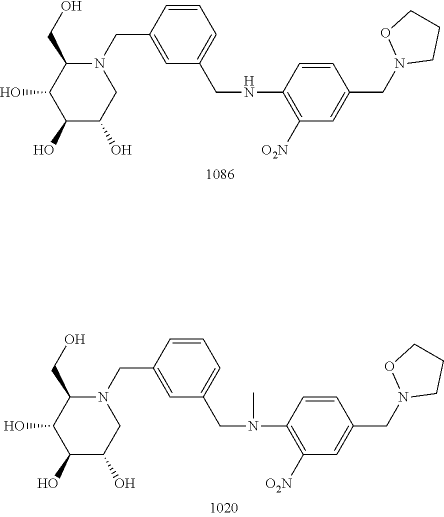 [SMILES: O=[N+]([O-])c1cc(CN2CCCO2)ccc1NCc1cccc(CN2C[C@H](O)[C@@H](O)[C@H](O)[C@H]2CO)c1 ; CN(Cc1cccc(CN2C[C@H](O)[C@@H](O)[C@H](O)[C@H]2CO)c1)c1ccc(CN2CCCO2)cc1[N+](=O)[O-]]

4-Fluoro-3-nitrophenyl)methanol (6 g), DCM (120 mL) were mixed at 0° C. then added TEA (3 eq) and MsCl (1.2 eq) at 0° C. After 1 hour the reaction was diluted with water and extracted with DCM (2×50 mL). The combined organic layers were washed with brine solution, dried over anhydrous Na₂SO₄, and concentrated to afford 6 g of 2.

2 (6 g), ACN (120 mL), K₂CO₃ (4.0 eq), isoxazolidine (1.5 eq) were mixed at room temperature then heated to reflux. After 20 hours the reaction was diluted with water then extracted with EtOAc (2×100 mL). The combined organic layers were washed with brine solution, dried over anhydrous Na₂SO₄, and concentrated, and purified on silica with 30% EtOAc in hexane to 100% EtOAc to afford 4 g of 4.

4 (4 g), ACN (80 mL), (3-(aminomethyl)phenyl)methanol (2 eq), K₂CO₃ (4 eq) were mixed and heated to 80° C. After 24 hours solvent was removed and the residue was diluted with water and extracted with EtOAc (2×40 mL). The combined organic layers were washed with brine solution, dried over anhydrous Na₂SO₄, concentrated, and purified on silica with 30% EtOAc in hexane to 100% EtOAc to afford 2.8 g of 6.

DCM (20 mL) and (COCl)₂ (3.0 eq) were mixed at −78° C., added DMSO (4 eq) for 30 minutes, mixed 6 (2 g) for 2 hours added Et₃N (5 eq), warmed to room temperature over 30 minutes, held 3 hours. The reaction was diluted with water and extracted with DCM (2×40 mL). The organic layer was dried over Na₂SO₄, filtered and concentrated to afford 1.8 g of 7.

7 (1.8 g), MeOH:DCM (180 mL, 1:1), DNJ (1.0 eq) were mixed at room temperature then added AcOH (cat.) and NaCNBH₃ (1.5 eq). After 72 hours the reaction was concentrated. The residue was diluted with water and extracted with 10% MeOH:DCM (4×40 mL). The combined organic layers were dried over anhydrous Na₂SO₄, concentrated, the material was triturated with Et₂O (2×50 mL) and EtOAc (2×30 mL) to afford 1 g of yellow solid. Two cycles of preparative HPLC purification and lyophilization yielded 418 mg of 1086 and 155 mg of 1020 as yellow solids.

### Example 17: Synthesis of Compound 1021 (2R,3R,4R,5S)-1-[(3-{[(3-bromo-5-methylphenyl)amino]methyl}phenyl)methyl]-2-(hydroxymethyl)piperidine-3,4,5-triol

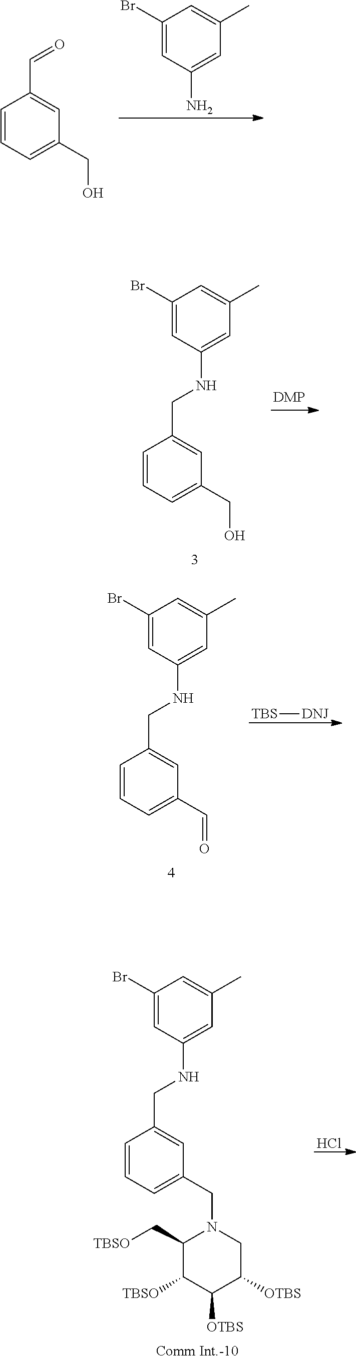

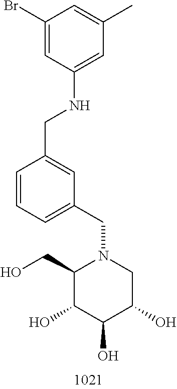

3-(Hydroxymethyl)-benzaldehyde (6 g), MeOH (500 mL), 3-bromo-5-methyl-aniline (1.0 eq) were mixed at 0° C., added AcOH (cat.), NaCNBH₃ (1.5 eq), temperature increased to room temperature. After 16 hours reaction volatiles were removed, residue was dissolved in water (250 mL) and extracted with EtOAc (2×250 mL). The organic layer was washed with water and dried over anhydrous Na₂SO₄ then concentrated and purified on silica with 15-20% EtOAc/hexane to afford 3 (5.5 g).

Mixed 3 (5.5 g) and DCM (600 mL) at 0° C., added DMP (1.0 eq) at 0° C. then stirred. After 15 minutes, the reaction was diluted with water and extracted with DCM. The organic layer was dried over anhydrous Na₂SO₄ then concentrated to afford 5 g of 4.

Mixed 4 (5 g), MeOH (8 mL), TBS-DNJ (0.7 eq) at 0° C., added AcOH (cat.), mixed 2 hours at 0° C., added NaCNBH₃ (1.5 eq) at 0° C., stirred 16 hours at room temperature. Reaction was concentrated, residue was dissolved in water (250 mL) and extracted with EtOAc (2×250 mL). The organic layer was washed with water, dried over anhydrous Na₂SO₄, concentrated, and purified on silica with 5-7% EtOAc/hexane to afford common Int-10 (8 g).

Common Int. 10 (4 g), DCM (40 mL) were mixed at 0° C. then 4.0 M HCl in 1,4-dioxane (2.5 mL) added at the same temperature which was then raised to room temperature. After 12 hours the reaction was concentrated, the material was washed with EtOAc and the solid obtained was dissolved in water (10 mL) and basified using saturated NH₄HCO₃. The water layer was decanted and residue was dissolved in methanol and evaporated to dryness to afford solid which was triturated with n-pentane to afford 1.8 g of 1021 as off white solid.

### Example 18: Synthesis of Compounds 1024 ((2R,3R,4R,5S)-1-[(4-{[(3-bromo-5-methylphenyl)amino]methyl}phenyl)methyl]-2-(hydroxymethyl)piperidine-3,4,5-triol) and 1050 ((2R,3R,4R,5S)-1-{[4-({[3-(5,6-dihydro-1,4-dioxin-2-yl)-5-methylphenyl]amino}methyl)phenyl]methyl}-2-(hydroxymethyl)piperidine-3,4,5-triol)

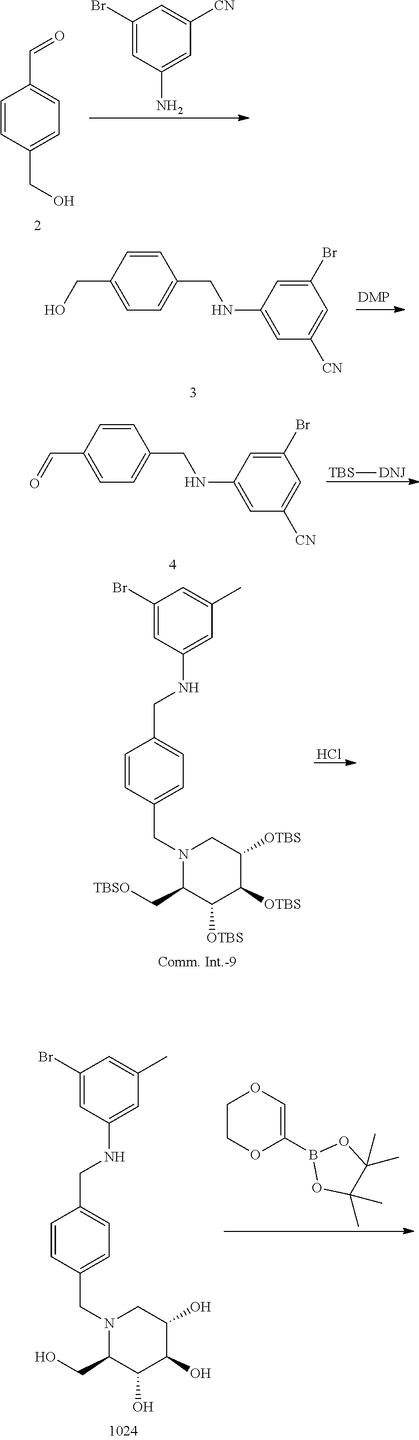

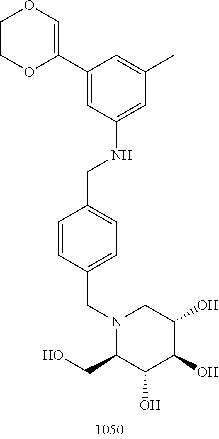 [SMILES: Cc1cc(NCc2ccc(CN3C[C@H](O)[C@@H](O)[C@H](O)[C@H]3CO)cc2)cc(C2=COCCO2)c1]

Mixed 2 (Example 2, 20 g), MeOH (500 mL) and 3-bromo-5-methylaniline (1.0 eq) at 0° C., added AcOH (cat.), NaCNBH₃ (1.5 eq) at 0° C., raised to room temperature. After 16 hours volatiles were removed, residue was dissolved in water (500 mL) and extracted with EtOAc (2×500 mL), organic layer was washed with water, dried over anhydrous Na₂SO₄, concentrated, and purified on silica using 30% EtOAc/hexane to afford 25 g of 3.

3 (25 g), DCM (2.5 L) were mixed at 0° C. then added DMP (1.2 eq) at same temperature and stirred for 15 minutes. The reaction was diluted with DCM and filtered through CELITE pad. The filtrate was washed with water and brine solution. The organic layer was dried over anhydrous Na₂SO₄ and concentrated to provide 4.

4 (25 g), MeOH (500 mL), TBS-DNJ (0.7 eq), AcOH (1 mL) were mixed at 0° C. for 2 hours then NaCNBH₃ (1.5 eq) added at 0° C. then stirred at room temperature. After 16 hours the reaction was concentrated, residue dissolved in water (1 L) and extracted with EtOAc (2×1 L). The organic layer was washed with water and dried over anhydrous Na₂SO₄ then concentrated and purified on silica [5-7% EtOAc/hexane] to afford common Int.-9 (30 g).

Common Int.-9 (5.0 g), MeOH (75 mL), DCM (25 mL) were mixed at 0° C. and 4.0 M HCl in 1,4-dioxane (100 mL) added at 0° C. then stirred at room temperature. After 16 hours volatiles were removed, and washed with EtOAc and filtered out the solid. The solid was suspended in water and basified with NH₄HCO₃ and concentrated, dissolved in 10% MeOH in DCM (100 mL) and filtered through CELITE bed and the filtrate was dried over anhydrous Na₂SO₄ and concentrated to afford 2.0 g of 1024.

Mixed 1024 (1 g), 1,4-dioxane (40 mL), H₂O (10 mL), 2-(5,6-dihydro-1,4-dioxin-2-yl)-boronate (2.0 eq) then Cs₂CO₃ (5.0 eq), degassed 15 minutes with N₂. Added Pd(dppf)Cl₂ (0.15 eq) at room temperature then heated to 100° C. After 16 hours volatiles were removed, material was dissolved in 10% MeOH in DCM (200 mL) and filtered through CELITE bed. The filtrate was dried over anhydrous Na₂SO₄, concentrated, and purified on silica using basic alumina (Al₂O₃) [20-30% MeOH in DCM with 2 mL of aq·NH₃]. The material was triturated with MeOH, EtOAc, and DCM in hexane. From this material 500 mg was purified by preparative HPLC to afford 200 mg of 1050 as off white solid.

### Example 19: Synthesis of Compound 1023 (3-bromo-5-{[(3-{[(2R,3R,4R,5S)-3,4,5-trihydroxy-2-(hydroxymethyl)piperidin-1-yl]methyl}phenyl)methyl]amino}benzonitrile)

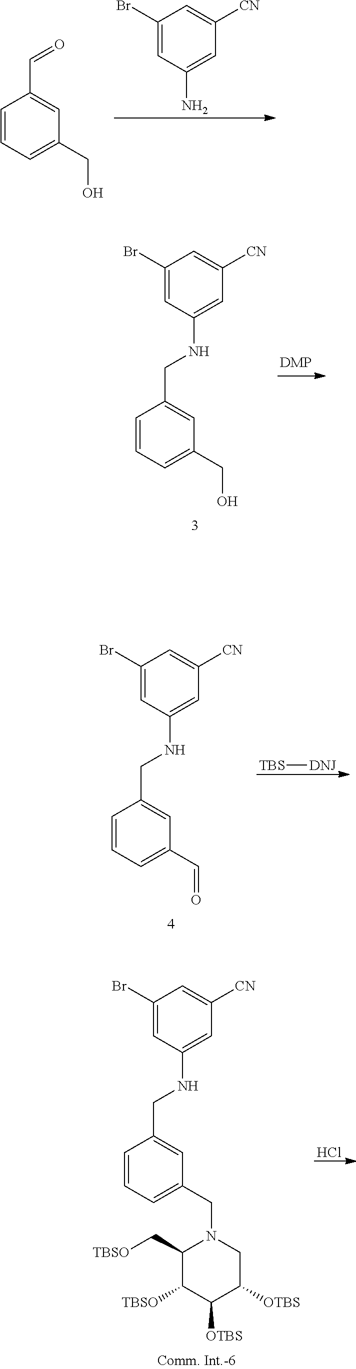

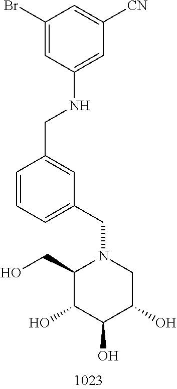

Mixed 3-(Hydroxymethyl)-benzaldehyde (4.0 g), MeOH (40 mL), 5-amino-3-bromo-benzonitrile (1.0 eq) at 0° C. then AcOH (cat.) and NaCNBH₃ (1.5 eq) added at the same temperature then warmed to room temperature. After 16 hours reaction volatiles were removed. The residue was dissolved in water (200 mL) and extracted with EtOAc (2×200 mL). The organic layer was washed with water, dried over anhydrous Na₂SO₄, concentrated, and purified on silica [15-20% EtOAc/hexane] to afford 3 (5.0 g).

To 3 (5.0 g) and DCM (50 mL) at 0° C. were added DMP (1.0 eq). After 15 minutes the reaction was diluted with water and extracted with DCM. The organic layer was dried over anhydrous Na₂SO₄ then concentrated to afford 5.5 g of 4.

Mixed 4 (5.5 g), MeOH (60 mL), TBS-DNJ (0.7 eq) at 0° C., added AcOH (cat.), 2 hours later added NaCNBH₃ (1.5 eq) and raised to room temperature. After 16 hours the reaction was concentrated, residue was dissolved in water (250 mL) and extracted with EtOAc (2×250 mL). The organic layer was washed with water, dried over anhydrous Na₂SO₄, concentrated, and purified on silica with 15-20% EtOAc/hexane to afford common Int.-6 (3.5 g).

Common Int-6 (300 mg), DCM (2 mL), and 4.0 M HCl in 1,4-dioxane (1 mL), were mixed at 0° C. for 4 hours. Volatiles were removed and the material was triturated with EtOAc and DCM to afford 30 mg of 1023 as off-white solid.

### Example 20: Synthesis of Compound 1022 ((2R,3R,4R,5S)-1-[(3-{[(3-bromo-5-methanesulfonylphenyl)amino]methyl}phenyl)methyl]-2-(hydroxymethyl)piperidine-3,4,5-triol)

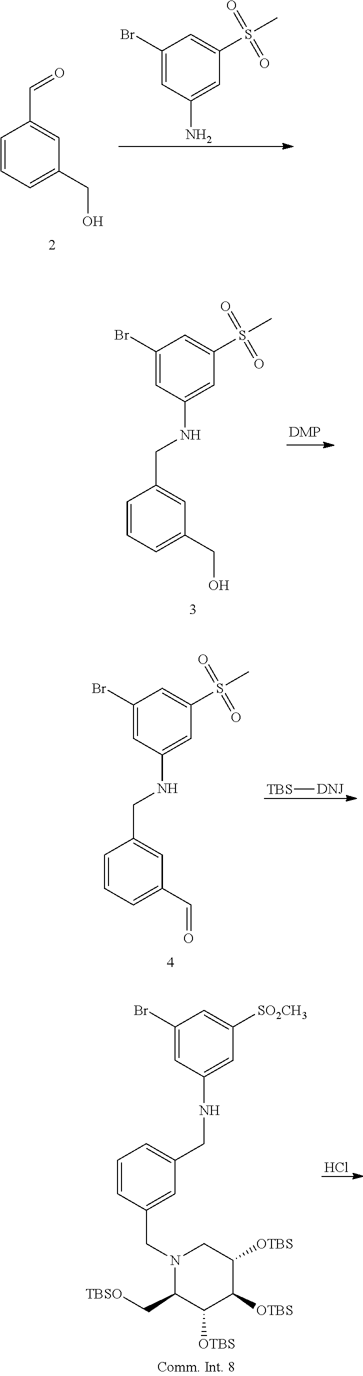

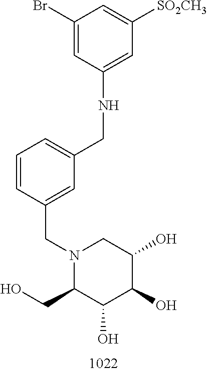 [SMILES: CS(=O)(=O)c1cc(Br)cc(NCc2cccc(CN3C[C@H](O)[C@@H](O)[C@H](O)[C@H]3CO)c2)c1]

Mixed 2 (10 g), 3-bromo-5-methylsulfonyl-aniline (1.2 eq), MeOH (1 L), AcOH (1 mL) at 0° C. then 30 minutes at room temperature, added NaCNBH₃ (1.5 eq) at 0° C. then raised to room temperature. After 16 hours volatiles were concentrated and residue solubilized in DCM and washed with water. The organic layer was dried over anhydrous Na₂SO₄, filtered, concentrated, and purified on silica with 50% EtOAc in hexane to afford 12.0 g of 3.

Mixed 3 (5 g) and DCM (500 mL) at 0° C., added DMP (1.0 eq) and increased to room temperature. After 30 minutes the reaction was filtered through CELITE, and the filtrate washed with water. The organic layer was dried over anhydrous Na₂SO₄ and concentrated to afford 5 g of 4.

Mixed 4 (5 g), MeOH (120 mL), TBS-DNJ (0.9 eq) at 0° C., added AcOH(cat.) and NaCNBH₃ (1.5 eq) and increased to room temperature. After 16 hours volatiles were concentrated, residue was dissolved in EtOAc, washed with water, dried over anhydrous Na₂SO₄, filtered, concentrated, and purified on silica [20% EtOAc-hexane] to give 3.5 g of common Int-8.

Mixed common Int-8 (300 mg) in DCM (4 mL) at 0° C., added 4.0 M HCl in 1,4-dioxane (2 mL) at 0° C. then increased to room temperature. After 16 hours volatiles were removed and residue triturated with DCM (20 mL). The solid was dissolved in MeOH (20 mL) which was then removed under reduced pressure to obtain 150 mg of 1022 as the HCl salt.

### Example 21: Synthesis of Compound 1025 ((2R,3R,4R,5S)-1-[(4-{[(3-bromo-5-methanesulfonyl phenyl)amino]methyl}phenyl)methyl]-2-(hydroxymethyl)piperidine-3,4,5-triol)

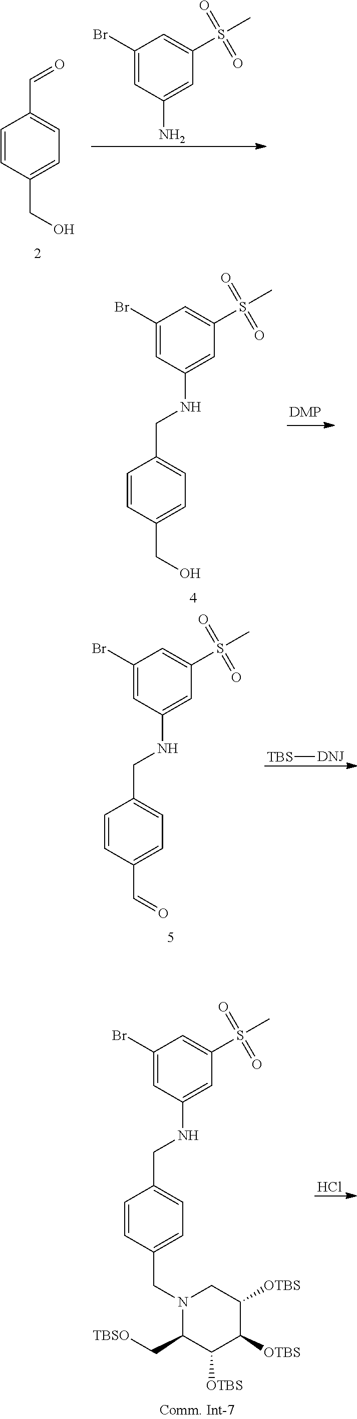

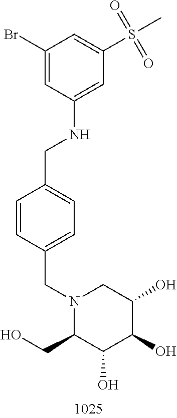 [SMILES: CS(=O)(=O)c1cc(Br)cc(NCc2ccc(CN3C[C@H](O)[C@@H](O)[C@H](O)[C@H]3CO)cc2)c1]

Mixed 3-bromo-5-(methylsulfonyl)aniline (10 g), 2 (1.5 eq), MeOH (200 mL), AcOH (2 mL) at 0° C. then at room temperature for 30 minutes. Added NaCNBH₃ (1.5 eq) d at 0° C. then temperature raised to room temperature. After 16 hours the volatiles were concentrated. Material was solubilized in DCM and washed with water, dried over anhydrous Na₂SO₄, filtered, concentrated, and purified on silica using 40% EtOAc in hexane to afford 10 g of 4.

Mixed 4 (9 g) and DCM (200 mL) at 0° C., added DMP (1.2 eq), increased to room temperature. After 2 hours the reaction was filtered through CELITE and the filtrate washed with water. The organic layer was dried over anhydrous Na₂SO₄ then concentrated to afford 9 g of 5.

5 (9 g), MeOH (200 mL), TBS-DNJ (1.0 eq), 0° C., AcOH (cat.), NaCNBH₃ (1.5 eq) mixed at room temperature. After 16 hours reaction volatiles were concentrated. The residue was dissolved in EtOAc and washed with water, dried over anhydrous Na₂SO₄, filtered, concentrated, and purified on silica [5% EtOAc in hexane] to afford 6.0 g of common Int-7.

Common Int-7 (300 mg), DCM (10 mL) mixed at 0° C., 4.0M HCl in 1,4-dioxane (2.5 mL) added then increased to room temperature. After 16 hours reaction volatiles were removed. Neutralization and trituration with EtOAc (10 mL) then Et₂O (20 mL) provided 25 mg of 1025.

### Example 22: Synthesis of Compound 1026 (3-bromo-5-{[(4-{[(2R,3R,4R,5S)-3,4,5-trihydroxy-2-(hydroxymethyl)piperidin-1-yl]methyl}phenyl)methyl]amino}benzonitrile)

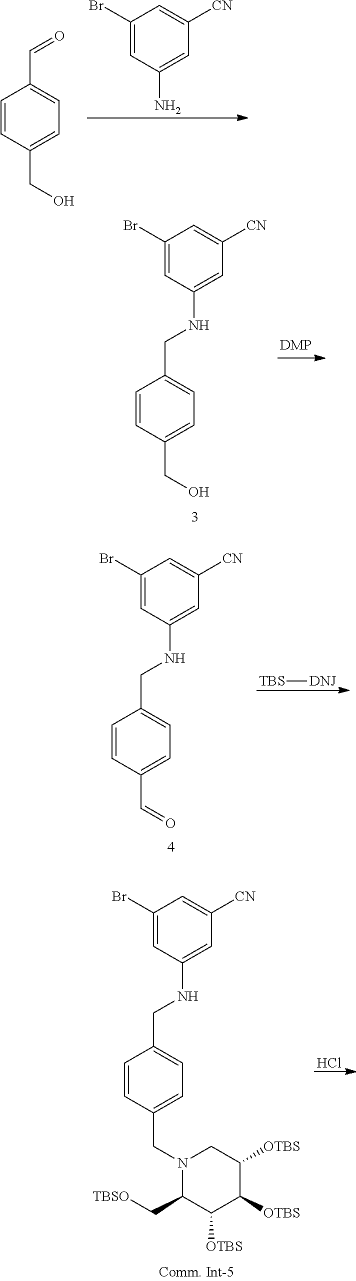

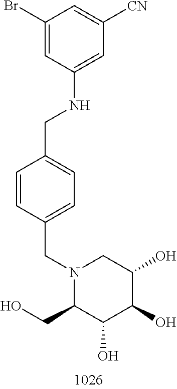 [SMILES: N#Cc1cc(Br)cc(NCc2ccc(CN3C[C@H](O)[C@@H](O)[C@H](O)[C@H]3CO)cc2)c1]

4-(Hydroxymethyl)-benzaldehyde (10 g), MeOH (100 mL), 3-amino-5-bromo-benzo-nitrile (1.0 eq) were mixed at 0° C. then added AcOH (cat.) and NaCNBH₃ (1.5 eq) at 0° C., then raised to room temperature. After 16 hours reaction volatiles were removed. The residue was dissolved in water (100 mL) and extracted with EtOAc (2×200 mL). The organic layer was washed with water and dried over anhydrous Na₂SO₄, concentrated, and purified on silica with 15-20% EtOAc/hexane to afford 3 (10 g).

3 (10 g), DCM (1.0 L) stirred at 0° C. then DMP (1.0 eq) added. After 15 minutes at 0° C. the reaction was diluted with water and extracted with DCM. The organic layer was dried over anhydrous Na₂SO₄ and concentrated to afford 10 g of 4.

Mixed 4 (10 g), MeOH (250 mL), TBS-DNJ (0.7 eq) at 0° C., added AcOH (cat.) at 0° C. for 2 hours added NaCNBH₃ (1.5 eq) at 0° C. then stirred at room temperature. After 16 hours reaction was concentrated, residue was dissolved in water (200 mL) and extracted with EtOAc (2×300 mL), organic layer was washed with water, dried over anhydrous Na₂SO₄, concentrated, and purified on silica with 15-20% EtOAc/hexane to afford 10.0 g of common Int-5.

Mixed Common Int-5 (300 mg) in DCM (3.0 mL) at 0° C., added 4.0 M HCl in 1,4-dioxane (1.5 mL) at 0° C., increased to room temperature. After 3 hours the reaction was concentrated, residue triturated with DCM for 10 minutes at room temperature, filtered and dried under high vacuum to obtain 1026 as off-white solid HCl salt. After lyophilization the salt was dissolved in a minimal amount of water and basified with aqueous NH₄HCO₃. The water was decanted and precipitate dissolved in MeOH, concentrated, triturated with EtOAc, and lyophilized to obtain 55.0 mg of 1026.

### Example 23: Synthesis of Compounds Derived from Common Int.-5

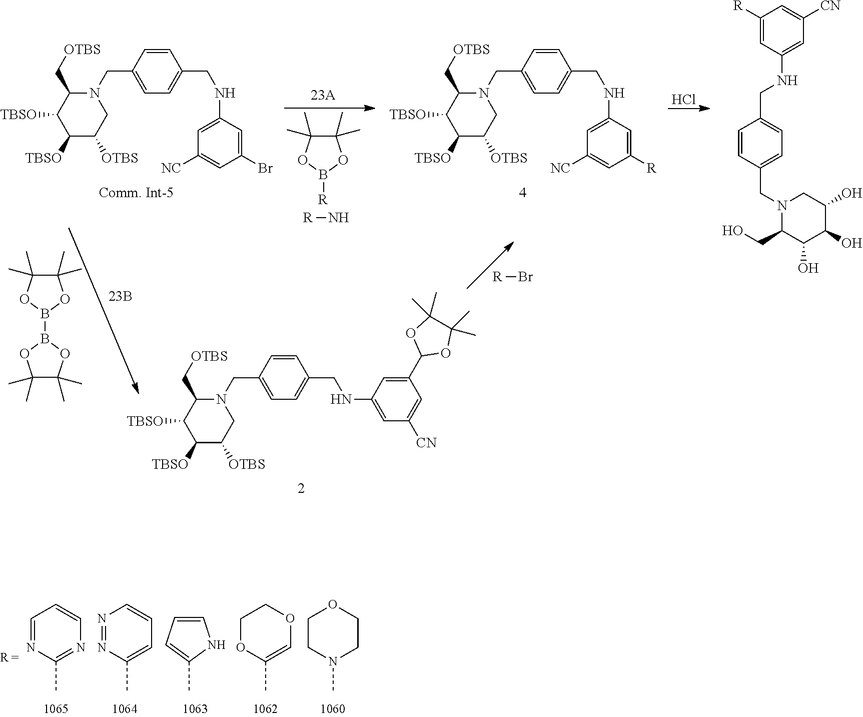

Common Int.-5 (Example 22) was converted to targets either by direct reaction (23A) with commercially available amine or boronate, or, (23B) converted to the boronate then coupled to commercially available bromide, followed in either case by de-protection.

For schemes 23B, common Int-5 (2.0 g), 1,4-dioxane (50 mL), bis(pinacolato) diboron (1.5 eq), KOAc (3.0 eq) were mixed and degassed for 15 minutes with N₂, then Pd(dppf)Cl₂ (0.1 eq) was added and the mixture heated to 100° C. After 16 hours reaction was diluted with water and extracted with EtOAc (2×150 mL). Organic layers were washed with brine solution, dried over anhydrous Na₂SO₄, filtered, concentrated to afford 2.0 g of 2.

Preparation of 1065 3-(pyrimidin-2-yl)-5-{[(4-{[(2R,3R,4R,5S)-3,4,5-trihydroxy-2-(hydroxymethyl)piperidin-1-yl]methyl}phenyl)methyl]amino}-benzonitrile (23B): Boronate 2 (1.5 g) in toluene:ethanol:water (1:1:1, 45 mL) 2-bromo-pyrimidine (1.0 eq), Na₂CO₃ (3.0 eq) were mixed and degassed for 15 minutes with N₂, then Pd(dppf)Cl₂ (0.1 eq) was added at room temperature then the mixture warmed to 80° C. After 16 hours reaction volatiles were removed and the material diluted with EtOAc and water. Separated the organic layer and dried over anhydrous Na₂SO₄ then concentrated and purified on silica with 15-20% EtOAc in hexane to afford 900 mg of 4.

Mixed 4 (900 mg) in DCM (10 mL) at 0° C., added 4.0 MHCl in 1,4-dioxane (5 mL), increased to room temperature. After 2 hours volatiles were removed, and residue triturated with EtOAc/DCM. The salt was dissolved in water and basified with aqueous NH₄HCO₃ to precipitate an off-white solid which was filtered and washed with n-pentane then triturated with EtOAc to afford 80 mg off white solid 1065.

Preparation of 1064 3-(pyridazin-3-yl)-5-{[(4-{[(2R,3R,4R,5S)-3,4,5-trihydroxy-2-(hydroxymethyl)piperidin-1-yl]methyl}phenyl)methyl]amino}-benzonitrile (23B): Boronate 2 (2.0 g), toluene:ethanol:water (1:1:1, 75 mL), 3-bromo-pyridazine (1.0 eq), Na₂CO₃ (3.0 eq) mixed and degassed for 15 minutes with N₂, added Pd(dppf)Cl₂ (0.1 eq) at room temperature then increased to 80° C. After 16 hours volatiles were removed and residue diluted with EtOAc and water. Separated the organic layer and dried over anhydrous Na₂SO₄ then concentrated and purified on silica with 30-40% EtOAc in hexane to afford 1.1 g of 4.

Mixed 4 (1.0 g) with DCM (10 mL) at 0° C., added 4.0 M HCl in 1,4-dioxane (5 mL) at 0° C. then increased to room temperature. After 2 hours volatiles were removed, the solid washed with DCM, the salt was dissolved in water and basified with aqueous NH₄HCO₃ to precipitate an off-white solid which was filtered and washed with n-pentane to afford 180 mg 1064.

Preparation of 1063 3-(1H-pyrrol-2-yl)-5-{[(4-{[(2R,3R,4R,5S)-3,4,5-trihydroxy-2-(hydroxymethyl)piperidin-1-yl]methyl}phenyl)methyl]amino}-benzonitrile (23A): Mixed common Int-5 (2 g), toluene:ethanol:water (1:1:1, 60 mL), 1-Boc-pyrrole-2-boronate (1.0 eq), Na₂CO₃ (3.0 eq), degassed 15 minutes with N₂, then Pd(dppf)Cl₂ (0.1 eq) added at room temperature then increased to 80° C. After 16 hours volatiles were removed, residue was dissolved in EtOAc (2×150 mL). The organic layer was washed with water and dried over anhydrous Na₂SO₄, concentrated, and purified on silica with 5-10% EtOAc/hexane to afford 1.5 g of 4.

4 (1.0 g), DCM (10.0 mL) were mixed at 0° C., 4.0 M HCl in 1,4-dioxane (5.0 mL) added at 0° C. then increased to room temperature. After 8 hours reaction volatiles were removed and preparative HPLC purification provided 110 mg of 1063.

Preparation of 1062 3-(5,6-dihydro-1,4-dioxin-2-yl)-5-{[(4-{[(2R,3R,4R,5S)-3,4,5-trihydroxy-2-(hydroxymethyl)piperidin-1-yl]methyl}phenyl)-methyl]amino}benzonitrile (23A): 1026 (Example 22, 1.7 g) and 2-(5,6-dihydro-1,4-dioxin-2-yl)-boronate (1 eq) with 1,4-dioxane:water (3:1) (100 mL) was purged with N₂ gas, mixed with Cs₂CO₃ (3.0 eq), Pd(dppf)Cl₂ (0.2 eq), and heated to 90° C. After 16 hours the reaction was cooled to room temperature and diluted with water and extracted with EtOAc (300 mL). The aqueous layer was saturated with NaCl and extracted with 15% MeOH/DCM (2×500 mL). The organic layer was dried over Na₂SO₄, filtered, concentrated to afford 1.5 g of product. Approximately 500 mg of this material was purified using preparative HPLC to afford 200 mg of 1062.

Preparation of 1060 3-(morpholin-4-yl)-5-{[(4-{[(2R,3R,4R,5S)-3,4,5-trihydroxy-2-(hydroxymethyl)piperidin-1-yl]methyl}phenyl)methyl]amino}-benzonitrile (23A): Mixed common Int-5 (2.5 g), 1,4-dioxane (60 mL), Pd₂(dba)₃ (0.2 eq), Xantphos (0.2 eq), degassed 15 minutes with N₂, morpholine (1.5 eq) then Cs₂CO₃ (3.0 eq) added at room temperature, increased to 100° C. After 16 hours reaction was cooled to room temperature and solvent removed, residue was dissolved in water and extracted with EtOAc (2×100 mL). The organic layer was dried over Na₂SO₄, filtered, distilled, and purified on silica [6% EtOAc in hexane] to afford 1.8 g of 4.

To 4 (1.9 g) in DCM (19 mL) at 0° C. added 4.0 MHCl in dioxane (9.5 mL) and warmed to room temperature. After 4 hours the reaction was concentrated and the material was triturated with EtOAc. The product was purified by preparative HPLC to obtain 70 mg of 1060.

### Example 24: Synthesis of Compounds Derived from Common Int.-6

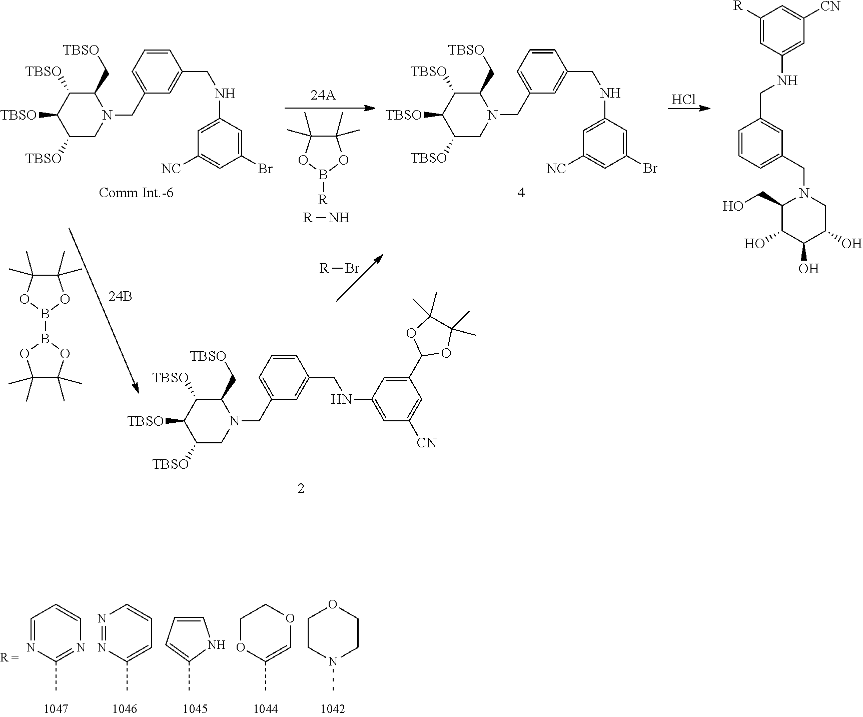

Common Int.-6 (Examples 19) was converted to targets either by direct reaction (24A) with commercially available amine or boronate, or, converted to the boronate ester (24B) and then coupled to commercially available bromide, followed in either case by de-protection.

For schemes 24B, in one example common Int-6 (1.0 g), 1,4-dioxane (15 mL), KOAc (3.0 eq), Pd(dppf)Cl₂ (0.2 eq) degassed for 15 minutes with N₂, then bis(pinacolato) diboron (1.5 eq) added at room temperature then increased to 100° C. After 16 hours the reaction was concentrated, residue was dissolved in water and extracted with EtOAc (2×100 mL). The organic layer was washed with water, dried over anhydrous Na₂SO₄, concentrated to give 1.4 g of 2.

Preparation of 1042 3-(morpholin-4-yl)-5-{[(3-{[(2R,3R,4R,5S)-3,4,5-trihydroxy-2-(hydroxymethyl)piperidin-1-yl]methyl}phenyl)methyl]amino}benzonitrile (24A): Common Int-6 (1.0 g), (3.0 eq), 1,4-dioxane (15 mL), Cs₂CO₃ (2.0 eq), Pd₂(dba)₃ (0.2 eq), Xantphos (0.2 eq) mixed and degassed for 15 minutes with N₂, then morpholine (1.5 eq) added at room temperature then increased to 100° C. After 16 hours the reaction was concentrated, the residue dissolved in water and extracted with EtOAc (2×150 mL). The organic layer was washed with water, dried over anhydrous Na₂SO₄, concentrated, and purified on silica with 15-20% EtOAc/hexane to afford 450 mg of 4.

4 (450 mg), DCM (4 mL), 4.0 MHCl in 1,4-dioxane (2 mL) mixed at 0° C. After 4 hours reaction volatiles were removed to afford 320 mg. The material was purified by preparative HPLC to afford 90 mg of 1042 as off white solid.

Preparation of 1044 3-(5,6-dihydro-1,4-dioxin-2-yl)-5-{[(3-{[(2R,3R,4R,5S)-3,4,5-trihydroxy-2-(hydroxymethyl)piperidin-1-yl]methyl}phenyl)methyl]amino}benzonitrile (24A): Deprotected common Int. 6 (1023, Example 19, 1.2 g) was mixed with 1,4-dioxane:water (3:1, 107 mL), Cs₂CO₃ (3 eq), Pd(dppf)Cl₂ (0.2 eq), 2-(5,6-dihydro-1,4-dioxin-2-yl)-boronate (2 eq), degassed 15 minutes with N₂, and heated to 90° C. After 16 hours the reaction was diluted with water and saturated with solid NaCl and extracted with 10% MeOH/DCM (2×200 mL). The organic layer was dried over Na₂SO₄, filtered, concentrated, and purification by preparative HPLC provided 298 mg of 1044.

Preparation of 1045 3-(1H-pyrrol-2-yl)-5-{[(3-{[(2R,3R,4R,5S)-3,4,5-trihydroxy-2-(hydroxymethyl)piperidin-1-yl]methyl}phenyl)methyl]amino}benzonitrile (24B): Common Int.-6 (1.5 g), toluene:ethanol:water (1:1:1, 15 mL), 1-Boc-pyrrole-2-boronate (1.0 eq), Na₂CO₃ (3.0 eq) degassed for 15 minutes with N₂, then Pd(dppf)Cl₂ (0.1 eq) added at room temperature then increased to 80° C. After 16 hours the reaction was concentrated. The residue was diluted with EtOAc and water, filtered through a pad of CELITE and washed with EtOAc. Combined organic layers were washed with water, dried over anhydrous Na₂SO₄, and concentrated, and purified on silica with 4% EtOAc/hexane to afford 4 (1.3 g).

4 (1.0 g), DCM (10 mL) mixed at 0° C., 4.0M HCl in dioxane (5 mL) added at 0° C. then increased to room temperature. After 8 hours reaction volatiles were removed and preparative HPLC purification and lyophilization gave 100 mg of 1045.

Preparation of 1046 3-(pyridazin-3-yl)-5-{[(3-{[(2R,3R,4R,5S)-3,4,5-trihydroxy-2-(hydroxymethyl)piperidin-1-yl]methyl}phenyl)methyl]amino}benzonitrile (24B): 2 (3.2 g), toluene:EtOH:water (1:1:1, 75 mL), Na₂CO₃ (3.0 eq), Pd(dppf)Cl₂ (0.2 eq), degassed for 15 minutes with N₂, then 3-bromopyridazine (1.5 eq) added at room temperature then heated to 80° C. After 16 hours the reaction was concentrated and the residue diluted with EtOAc and water, filtered through a pad of CELITE, washed with EtOAc. Combined organic layers were washed with water then dried over anhydrous Na₂SO₄, concentrated, and purified on silica [30-40% EtOAc/hexane] to afford 4 (2.0 g).

4 (2.0 g), DCM (20 mL) mixed at 0° C., add 4.0 M HCl in 1,4-dioxane (10 mL) at 0° C. then increased to room temperature. After 2 hours volatiles were removed, residue suspended in water and basified using aq. NH₄HCO₃, precipitate was dissolved in methanol, distilled, triturated with EtOAc (2×4 mL) to afford 350 mg 1046 as off white solid.

Preparation of 1047 3-(pyrimidin-2-yl)-5-{[(3-{[(2R,3R,4R,5S)-3,4,5-trihydroxy-2-(hydroxymethyl)piperidin-1-yl]methyl}phenyl)methyl]amino}benzonitrile (24B): Degassed 2 (1.4 g), toluene:EtOH:water (1:1:1, 24 mL), Pd(dppf)Cl₂ (0.2 eq), and Na₂CO₃ (3.0 eq), 15 minutes with N₂, then 2-bromopyrimidine (1.5 eq) added at room temperature and heated to 100° C. After 16 hours volatiles were removed and residue was dissolved in water and extracted with EtOAc (2×200 mL). The organic layer was washed with water, dried over anhydrous Na₂SO₄, concentrated, and purified on silica with 15-20% EtOAc/hexane to afford 4 (600 mg).

Mixed 4 (600 mg), DCM (6 mL), 4.0 M HCl in 1,4-dioxane (3 mL) at 0° C., increased to room temperature. After 4 hours volatiles were removed, material was triturated with EtOAc with 3 drops of MeOH to afford the HCl salt as yellow solid. The solid was basified with saturated NH₄CO₃ solution and the precipitated solid was washed with EtOAc, filtered and dried to afford 82 mg of 1047 off white solid after lyophilization.

### Example 25: Synthesis of Compounds Derived from Common Int.-7

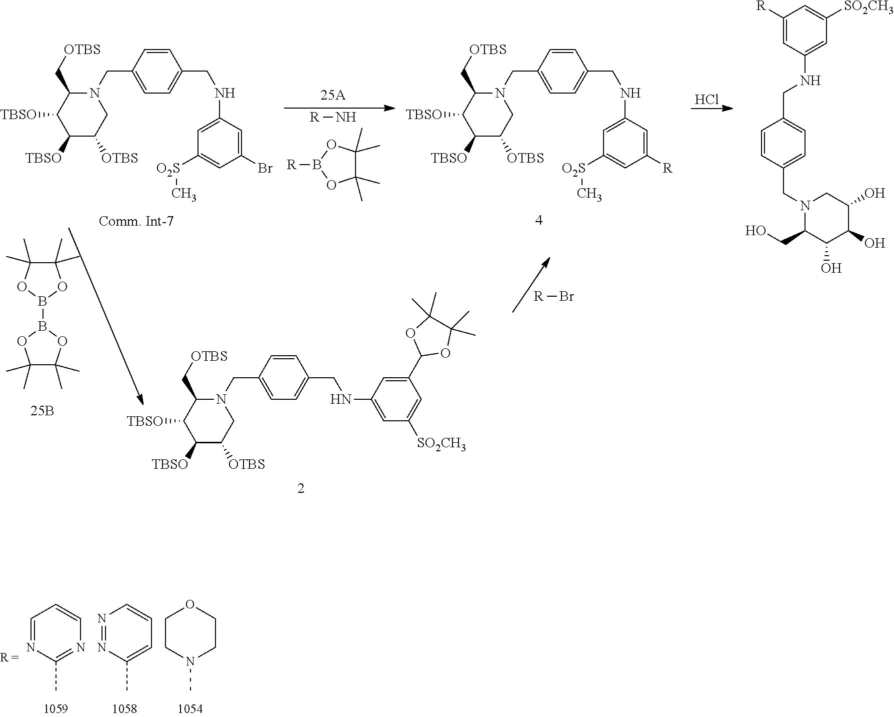

Common Int.-7 (Example 21) was converted to targets either by direct reaction (25A) with commercially available amine or boronate, or, converted to the boronate ester (25B) and then coupled to commercially available bromide, followed in either case by de-protection.

For schemes 25B, in one example common Int-7 (2.0 g), 1,4-dioxane (40 mL), bis-pinacolato-diboron (1.5 eq), KOAc (3.0 eq), degassed for 15 minutes with N₂, Pd(dppf)Cl₂ (0.1 eq) added and heated to 100° C. After 16 hours reaction was diluted with water, extracted with EtOAc (2×100 mL), organic layers were washed with brine solution, dried over anhydrous Na₂SO₄, filtered, concentrated to afford 2.0 g of boronate 2.

Preparation of 1054 (2R,3R,4R,5S)-2-(hydroxymethyl)-1-{[4-({[3-methanesulfonyl-5-(morpholin-4-yl)phenyl]amino}methyl)phenyl]methyl}piperidine-3,4,5-triol (25A): Common Int-7 (1.0 g), 1,4-dioxane (30 mL), Cs₂CO₃ (3.0 eq), Xantphos (0.2 eq), Pd₂(dba)₃ (0.2 eq), degassed with N₂ for 10 minutes, added morpholine (6.0 eq) and increased to 100° C. After 16 hours reaction volatiles were removed, the residue was diluted with water, extracted with EtOAc, the organic dried over anhydrous Na₂SO₄, filtered, concentrated, and purified on silica [20% EtOAc-hexane] to afford 500 mg of 4.

4 (500 mg), DCM (10 mL) mixed at 0° C., 4.0 MHCl in 1,4 dioxane (2 mL) added at 0° C. then increased to room temperature. After 16 hours volatiles were removed, the residue was washed with Et₂O, the salt was neutralized with saturated NaHCO₃, extracted with EtOAc, dried over anhydrous Na₂SO₄, and concentrated to afford 106 mg of 1054 as off-white solid.

Preparation of 1059 (2R,3R,4R,5S)-2-(hydroxymethyl)-1-{[4-({[3-methanesulfonyl-5-(pyrimidin-2-yl)phenyl]amino}methyl)phenyl]methyl}piperidine-3,4,5-triol (25B): Common Int.-7 (1.0 g), toluene:ethanol:water (1:1:1, 30 mL), 2-bromo-pyrimidine (2.0 eq), Na₂CO₃ (3.0 eq), degassed 15 minutes with N₂, added Pd(dppf)Cl₂ (0.1 eq) at room temperature then increased to 80° C. After 16 hours volatiles were removed. The residue was diluted with water and extracted with EtOAc. The organic layer was dried over anhydrous Na₂SO₄, filtered, concentrated, and purified on silica 20% EtOAc in hexane to afford 700 mg of 4.

4 (700 mg), DCM (15 mL), mixed at 0° C., 4.0M HCl in 1,4-dioxane (6 mL) added and increased to room temperature. After 16 hours volatiles were removed, residue washed with Et₂O and dried to afford 350 mg of 1059 as the HCl salt as light yellow solid.

Preparation of 1058 (2R,3R,4R,5S)-2-(hydroxymethyl)-1-{[4-({[3-methanesulfonyl-5-(pyridazin-3-yl)phenyl]amino}methyl)phenyl]methyl}piperidine-3,4,5-triol (25B): Common Int.-7 (1.0 g), toluene:ethanol:water (1:1:1, 30 mL), 3-bromo-pyridazine (2.0 eq), Na₂CO₃ (3.0 eq), degassed for 15 minutes with N₂, Pd(dppf)Cl₂ (0.1 eq) added at room temperature then increased to 80° C. After 16 hours reaction volatiles were removed, the residue was diluted with water and extracted with EtOAc, the organic was dried over anhydrous Na₂SO₄, filtered, concentrated, and purified on silica [20% EtOAc-hexane] to afford 600 mg of 4.

4 (600 mg), DCM (12 mL), mixed at 0° C., 4.0 M HCl in 1,4-dioxane (6 mL) added at room temperature. After 16 hours reaction volatiles were removed, and residue triturated with Et₂O and dried to afford 311 mg of 1058 as HCl salt as light yellow solid.

### Example 26: Synthesis of Compounds Derived from Common Int.-8

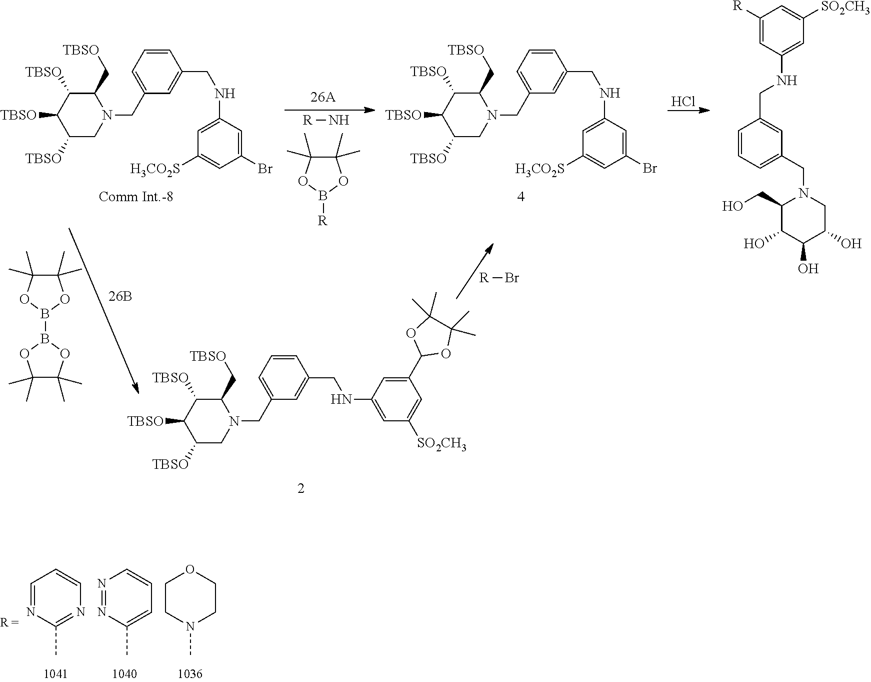

Common Int.-8 (Example 20) was converted to targets either by direct reaction (26A) with commercially available amine or boronate, or, converted to the boronate ester (26B) and then coupled to commercially available bromide, followed in either case by de-protection.

For schemes 26B, in one example common Int.-8 (2 g), 1,4-dioxane (40 mL), bis-pinacolato-diboron (1.5 eq), KOAc (3.0 eq) mixed and degassed for 15 minutes with N₂, then Pd(dppf)Cl₂ (0.1 eq) added and temperature increased to 100° C. After 16 hours reaction was diluted with water, extracted with EtOAc (2×50 mL), organic layers were washed with brine, dried over anhydrous Na₂SO₄, filtered, concentrated to afford 2 g of 2.

Preparation of 1036 (2R,3R,4R,5S)-2-(hydroxymethyl)-1-{[3-({[3-methanesulfonyl-5-(morpholin-4-yl)phenyl]amino}methyl)phenyl]methyl}piperidine-3,4,5-triol (26A): Common Int.-8 (2.0 g), 1,4-dioxane (100 mL), Cs₂CO₃ (2.0 eq), Xantphos (0.2 eq), Pd₂(dba)₃ (0.2 eq), mixed and degassed with N₂ for 10 minutes, then morpholine (4.0 eq) added and temperature increased to 100° C. After 16 hours reaction volatiles were removed. The residue was diluted with water and extracted with EtOAc. The organic layer was dried over anhydrous Na₂SO₄, filtered, concentrated, and purified on silica with 25% EtOAc in hexane to afford 1.2 g of 4.

4 (1.2 g), DCM (12 mL) mixed at 0° C., 4.0 M HCl in 1,4 dioxane (6 mL) added at 0° C. then increased to room temperature. After 16 hours volatiles were removed, residue washed with DCM (30 mL) and filtered. The material was dissolved in MeOH (30 mL) and concentrated. This salt was dissolved in water (15 mL) and basified by using saturated NH₄HCO₃. The water layer was decanted and residue was dissolved in 10% methanol in DCM and evaporated to dryness then washed with EtOAc (15 mL) and filtered to yield pale brown solid which after lyophilization provided 170 mg of 1036.

Preparation of 1040 (2R,3R,4R,5S)-2-(hydroxymethyl)-1-{[3-({[3-methanesulfonyl-5-(pyridazin-3-yl)phenyl]amino}methyl)phenyl]methyl}piperidine-3,4,5-triol (26B): 2 (1 g), toluene:ethanol:water (1:1:1, 30 mL), 3-bromo-pyridazine (2.0 eq), Na₂CO₃ (3.0 eq), degassed for 15 minutes with N₂, added Pd(dppf)Cl₂ (0.1 eq) at room temperature then increased to 80° C. After 4 hours reaction volatiles were removed, the residue was diluted with water and extracted with EtOAc. The organic layer was dried over anhydrous Na₂SO₄, filtered, and concentrated, and purified on silica with 20% EtOAc in hexane to afford 580 mg of 4.

4 (580 mg), DCM (15 mL) mixed at 0° C., added 4.0 M HCl in 1,4-dioxane (5.8 mL) and increased to room temperature. After 16 hours the reaction volatiles were removed, and residue triturated with EtOAc (2×20 mL), Et₂O (2×30 mL) and dried to afford 350 mg of 1040 as yellow HCl salt.

Preparation of 1041 (2R,3R,4R,5S)-2-(hydroxymethyl)-1-{[3-({[3-methanesulfonyl-5-(pyrimidin-2-yl)phenyl]amino}methyl)phenyl]methyl}piperidine-3,4,5-triol (26B): 2 (1 g), toluene:ethanol:water (1:1:1, 30 mL), 2-bromo-pyrimidine (2.0 eq), Na₂CO₃ (3.0 eq), degassed for 15 minutes with N₂, Pd(dppf)Cl₂ (0.1 eq) added at room temperature then increased to 80° C. After 4 hours volatiles were removed, residue was diluted with water and extracted with EtOAc. The organic layer was dried over anhydrous Na₂SO₄, filtered, concentrated, and purified on with 20% EtOAc in hexane to afford 600 mg of 4.

4 (600 mg), DCM (15 mL) mixed at 0° C., added 4.0 M HCl in 1,4-dioxane (6 mL), increased to room temperature. After 16 hours reaction volatiles were removed. The material was triturated with EtOAc (2×20 mL), Et₂O (2×30 mL), and dried to afford 275 mg of 1041 as yellow HCl salt.

### Example 27: Synthesis of Compounds Derived from Common Int.-9

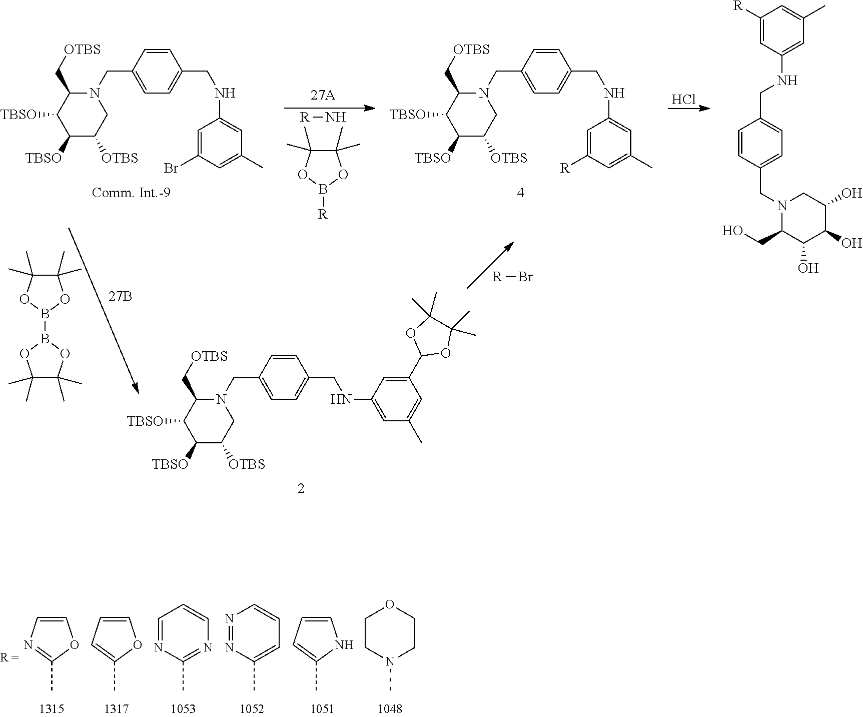

Common Int.-9 (Example 18) was converted to targets either by direct reaction (27A) with commercially available amine or boronate, or, converted to the boronate ester (27B) and then coupled to commercially available bromide, followed in either case by de-protection.

Preparation of 1053 (2R,3R,4R,5S)-2-(hydroxymethyl)-1-{[4-({[3-methyl-5-(pyrimidin-2-yl)phenyl]amino}methyl)phenyl]methyl}piperidine-3,4,5-triol (27B): Common Int.-9 (4.5 g), 1,4-dioxane (90 mL), bis-pinacolato diboron (1.5 eq), KOAc (3.0 eq), were mixed and degassed for 15 minutes with N₂, then Pd(dppf)Cl₂ (0.1 eq) added and temperature increased to 100° C. After 16 hours reaction was diluted with water and extracted with EtOAc (2×250 mL). The combined organic layers were washed with brine solution, dried over anhydrous Na₂SO₄, filtered, concentrated to afford 5.0 g of 2.

Boronate 2 (2.5 g), toluene:ethanol:water (1:1:1, 75 mL), 2-bromo-pyrimidine (2.0 eq), Na₂CO₃ (3.0 eq) were mixed and degassed for 15 minutes with N₂, then added Pd(dppf)Cl₂ (0.1 eq) at room temperature then increased to 80° C. After 16 hours reaction volatiles were removed and diluted with water and extracted with EtOAc (2×100 mL). The combined organic layers were washed with brine solution, dried over anhydrous Na₂SO₄, filtered, concentrated, and purified on silica with 5-10% EtOAc in hexane to afford 1.4 g of 4.

4 (1.5 g), DCM (30 mL), 4.0 M HCl in 1,4-dioxane (10 mL), added at 0° C. then stirred at room temperature. After 16 hours the reaction volatiles were removed, residue washed with Et₂O and dissolved in water and basified with aq. NH₄HCO₃. The aqueous layer was concentrated and material was taken up in 10% MeOH in DCM (20 mL), solid was filtered out and the filtrate was dried over anhydrous Na₂SO₄, filtered, concentrated, and triturated with EtOAc, followed by Et₂O to afford 500 mg of 1053 as off white solid.

Preparation 1051 (2R,3R,4R,5S)-2-(hydroxymethyl)-1-{[4-({[3-methyl-5-(1H-pyrrol-2-yl)phenyl]amino}methyl)phenyl]methyl}piperidine-3,4,5-triol (27A): Common Int.-9 (5.0 g), toluene:ethanol:water (1:1:1, 100 mL), 1-Boc-pyrrole-2-boronate (1.0 eq), Na₂CO₃ (3.0 eq), degassed 15 minutes with N₂, Pd(dppf)Cl₂ (0.1 eq) added at room temperature then increased to 80° C. After 16 hours reaction volatiles were removed and product diluted with water and extracted with EtOAc (2×100 mL). The combined organic layers were washed with brine solution, dried over anhydrous Na₂SO₄, filtered, concentrated, and purified on silica with 2% EtOAc/hexane to afford 3.5 g of 4.

4 (3.5 g), MeOH (30 mL) mixed at 0° C., added 4.0 M HCl in 1,4-dioxane (17 mL), increased to room temperature. After 16 hours volatile solvents were removed and after triturations with EtOAc and Et₂O and purification by preparative HPLC afforded 120 mg of 1051.

Preparation of 1052 (2R,3R,4R,5S)-2-(hydroxymethyl)-1-{[4-({[3-methyl-5-(pyridazin-3-yl)phenyl]amino}methyl)phenyl]methyl}piperidine-3,4,5-trio (27B)₁: Degassed 2 (2.5 g), toluene:ethanol:water (1:1:1, 75 mL), 3-bromo-pyridazine (2.0 eq), Na₂CO₃ (3.0 eq) 15 minutes with N₂, Pd(dppf)Cl₂ (0.1 eq) added at room temperature then increased to 80° C. After 16 hours reaction volatiles were removed and the material diluted with water and extracted with EtOAc (2×100 mL). Combined organic layers were washed with brine solution, dried over anhydrous Na₂SO₄, filtered, concentrated. Material was purified on silica with 5-10% EtOAc in hexane to afford 1.5 g of 4.

4 (1.5 g), DCM (30 mL), 4.0 M HCl in 1,4-dioxane (10 mL), added at room temperature then stirred. After 16 hours reaction volatiles were removed, and residue washed with Et₂O and dissolved in water and basified with aq. NH₄HCO₃. The aqueous layer was concentrated and the material was taken up in 10% MeOH in DCM (20 mL). Solid was filtered out and the filtrate was dried over anhydrous Na₂SO₄, filtered, concentrated, and residue washed with EtOAc followed by Et₂O and dried to afford 500 mg 1052 as off white solid.

Preparation of 1048 (2R,3R,4R,5S)-2-(hydroxymethyl)-1-{[4-({[3-methyl-5-(morpholin-4-yl)phenyl]amino}methyl)phenyl]methyl}piperidine-3,4,5-triol (27A): Mixed common Int.-9 (5.0 g), toluene (100 mL), with morpholine (6.0 eq) then NaOtBu (3.0 eq), degassed for 15 minutes with N₂, then Pd₂(dba)₃ (0.2 eq) and BINAP (0.2 eq) addition at room temperature then increased to 100° C. After 16 hours reaction volatiles were removed, residue was diluted with water and extracted with EtOAc (3×150 mL). Combined organic layers were washed with brine solution, dried over anhydrous Na₂SO₄, filtered, concentrated, and purified on silica with 5-10% EtOAc in hexane to afford 1.0 g of 4.

4 (1.0 g), DCM (50 mL) mixed at 0° C., 4.0M HCl in 1,4-dioxane (10 mL) addition at 0° C. then warmed to room temperature. After 16 hours reaction volatiles were removed, and residue washed with EtOAc. Filtered the solid which was taken in water and basified with NH₄HCO₃ and concentrated, and dissolved in 10% MeOH in DCM (100 mL) and filtered through CELITE bed and the filtrate dried over anhydrous Na₂SO₄ and concentrated. Preparative HPLC and lyophilization gave 140 mg of 1048.

Preparation of 1315 (2R,3R,4R,5S)-2-(hydroxymethyl)-1-{[4-({[3-methyl-5-(1,3-oxazol-2-yl)phenyl]amino}methyl)phenyl]methyl}piperidine-3,4,5-triol (27B): Degassed 2 (500 mg), toluene:EtOH:water (1:1:1, 30 mL), 2-bromooxazole (2.5 eq), and Na₂CO₃ (3 eq) 15 minutes with N₂, added Pd(dppf)Cl₂ (0.25 eq), reacted 16 hours at 80° C. Mixture was dissolved in water and extracted with EtOAc (2×50 mL), organic layer was dried over anhydrous Na₂SO₄, filtered, concentrated, and purified on silica [2% EtOAc in hexane] to afford 280 mg of 4.

Mixed 4 (280 mg), MeOH:DCM (1:1, 6 mL), 4 M HCl in 1,4-dioxane (2.8 mL) at 0° C. then at room temperature for 16 hours. Volatiles were removed and the obtained material was purified by preparative HPLC to afford 29 mg of 1315.

Preparation of 1317 (2R,3R,4R,5S)-1-{[4-({[3-(furan-2-yl)-5-methylphenyl]amino}methyl)phenyl]methyl}-2-(hydroxymethyl)piperidine-3,4,5-triol (27A): An initial 20 mg of 4 was obtained from 50 mg common Int.-9 using the reaction conditions shown for 1051. In a separate reaction, degassed toluene:MeOH (4:1, 40 mL) and common Int-9 (400 mg) 30 minutes under N₂, added 2-furanyl-boronate (1.5 eq), Cs₂CO₃ (3.0 eq), Pd(PPh₃)₄ (0.1 eq) at room temperature then mixed 16 hours at 80° C. Reaction was dissolved in water and extracted with EtOAc (2×100 mL), organic layer was dried over anhydrous Na₂SO₄, filtered, concentrated, and purified by COMBIFLASH [hexane] to afford 280 mg of 4.

Mixed 4 (280 mg), DCM, (10 mL), 4.0 M HCl in 1,4-dioxane (5 mL) at 0° C. then at room temperature for 6 hours. Reaction volatiles were removed and residue triturated with DCM then EtOAc, then preparative HPLC purification and lyophilization gave 40 mg of 1317.

### Example 28: Synthesis of Compounds Derived from Common Int.-10

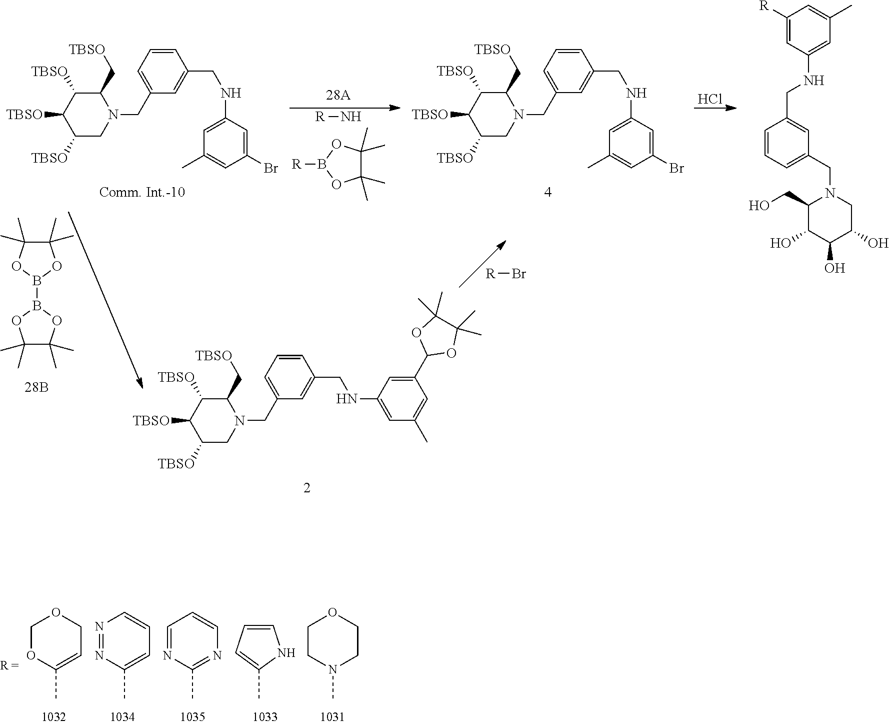

Common Int.-10 (Example 17) was converted to targets either by direct reaction (28A) with commercially available amine or boronate, or, converted to the boronate ester (28B) and then coupled to commercially available bromide, followed in either case by de-protection.

For schemes 28B, in one example common Int.-10 (3.5 g), 1,4-dioxane (80 mL), bis-pinacolato diboron (1.5 eq.), KOAc (3.0 eq.) degassed for 15 minutes with N₂, added Pd(dppf)Cl₂ (0.1 eq), heated to 100° C. After 6 hours reaction was diluted with water and extracted with EtOAc (2×500 mL). Combined organic layers were washed with brine, dried over anhydrous Na₂SO₄, filtered, concentrated to afford 3.5 g of boronate 2.

Preparation of 1033 (2R,3R,4R,5S)-2-(hydroxymethyl)-1-{[3-({[3-methyl-5-(1H-pyrrol-2-yl)phenyl]amino}methyl)phenyl]methyl}piperidine-3,4,5-triol (28A): Mixed Common Int.-10 (3.0 g), toluene:ethanol:H₂O (1:1:1, 90 mL), 1-Boc-pyrrole-2-boronate (1.0 eq), Na₂CO₃ (3.0 eq) and degassed 15 minutes with N₂, added Pd(dppf)Cl₂ (0.1 eq) at room temperature then heated at 80° C. 16 hours. Concentrated the reaction, diluted the residue with EtOAc and water, filtered through a pad of CELITE, washed with EtOAc. Organic layers were washed with water, dried over anhydrous Na₂SO₄, filtered, concentrated, and purified on silica using 4% EtOAc/hexane to afford 4 (2.2 g).

Mixed b (1.1 g) in DCM:MeOH (1:1, 20 mL) at 0° C., added 4.0 M HCl in dioxane (15 mL), increased to room temperature. After 6 hours reaction volatiles were removed, residue was washed with Et₂O and EtOAc, and reaction mass was suspended in water and basified with aqueous saturated NaHCO₃. The aqueous layer was extracted with EtOAc, dried over anhydrous Na₂SO₄, filtered, concentrated, and washed with MTBE. Filtrate was concentrated to afford additional material. Materials were purified by preparative HPLC, combined and lyophilized to afford 140 mg of 1033. An alternative preparation at larger scale in Example 3.

Preparation of 1032 (2R,3R,4R,5S)-1-{[3-({[3-(5,6-dihydro-1,4-dioxin-2-yl)-5-methylphenyl]amino}methyl)phenyl]methyl}-2-(hydroxymethyl)piperidine-3,4,5-triol (28A): Deprotection (Example 17) was carried out prior to the coupling reaction, then 1021 (1.75 g), 1,4-dioxane:H₂O (3:1, 48 mL), 2-(5,6-dihydro-1,4-dioxin-2-yl)-boronate (2.0 eq) were mixed, added Cs₂CO₃ (5.0 eq), degassed 15 minutes with N₂, added Pd(dppf)Cl₂ (0.15 eq) at room temperature then mixed 16 hours at 100° C. Cooled to room temperature. Volatiles were removed to afford residue which was suspended in 10% MeOH/DCM. The solids were filtered out through a pad of CELITE and the filtrate was washed with saturated NaCl solution. The organic layer was dried over Na₂SO₄, filtered, distilled, purified by preparative HPLC and lyophilized to obtain 250 mg of 1032.

Preparation of 1031 (2R,3R,4R,5S)-2-(hydroxymethyl)-1-{[3-({[3-methyl-5-(morpholin-4-yl)phenyl]amino}methyl)phenyl]methyl}piperidine-3,4,5-triol (28A): Degassed common Int-10 (1 g), toluene (10 mL), morpholine (4.0 eq), t-BuONa (1.5 eq) 15 minutes with N₂, followed by BINAP (0.2 eq) and Pd₂(dba)₃ (0.1 eq) addition at room temperature then increased to 80° C. After 6 hours the reaction was concentrated. The residue was diluted with EtOAc, washed with water, dried over anhydrous Na₂SO₄, concentrated. After column purification 700 mg of 4 was obtained.

b (700 mg), DCM (4 mL) were mixed at 0° C. then 4.0 M HCl in dioxane (2 mL) added and increased to room temperature. After 16 hours volatiles were removed, and residue washed with DCM (30 mL) and filtered. The solid was dissolved in MeOH (30 mL) and the solvent was removed. The solid was dissolved in water (15 mL) and basified with saturated NH₄HCO₃. The water layer was decanted and residue dissolved in 10% methanol in DCM and evaporated to dryness. This solid was further washed with EtOAc (15 mL) and filtered. After lyophilization 100 mg of 1031 was obtained with an additional 130 mg showing EtOAc solvent by ¹H-NMR which was not combined.

Preparation of 1034 (2R,3R,4R,5S)-2-(hydroxymethyl)-1-{[3-({[3-methyl-5-(pyridazin-3-yl)phenyl]amino}methyl)phenyl]methyl}piperidine-3,4,5-triol (28B): Mixed 2 (3.8 g), toluene:ethanol:water (1:1:1, 100 mL), 3-bromo-pyridazine (2.0 eq), Na₂CO₃ (3.0 eq), degassed 15 minutes with N₂, added Pd(dppf)Cl₂ (0.1 eq) at room temperature then increased to 80° C. After 16 hours reaction volatiles were removed. The residue was diluted with water and extracted with EtOAc (2×200 mL). Combined organic layers were washed with brine solution, dried over anhydrous Na₂SO₄, filtered, concentrated, and purified on silica with 35% EtOAc/hexane to afford 1.5 g of 4.

4 (1.5 g), DCM (20 mL), 4.0 M HCl in 1,4-Dioxane (10 mL), mixed at room temperature. After 16 hours volatiles were removed, and residue triturated with DCM (70 mL) and filtered the solid which was dissolved in MeOH (30 mL). The solvent was removed and this salt was neutralized using saturated NH₄HCO₃. The precipitate was filtered and washed with EtOAc to obtain 600 mg of 1034 as brown solid.

Preparation of 1035 (2R,3R,4R,5S)-2-(hydroxymethyl)-1-{[3-({[3-methyl-5-(pyrimidin-2-yl)phenyl]amino}methyl)phenyl]methyl}piperidine-3,4,5-triol (28B): Mixed 2 (2.8 g), toluene:ethanol:water (1:1:1, 50 mL), 2-bromo-pyrimidine (2.0 eq), Na₂CO₃ (3.0 eq), degassed for 15 minutes with N₂, added Pd(dppf)Cl₂ (0.1 eq) at room temperature then reacted at 80° C. After 16 hours volatiles were removed, the residue was diluted with water and extracted with EtOAc (2×100 mL). Combined organic layers were washed with brine solution, dried over anhydrous Na₂SO₄, filtered, concentrated, and purified on silica with 10% EtOAc in hexane to afford 1 g of 4.

Mixed 4 (1 g), DCM (10 mL), 4.0 M HCl in 1,4-dioxane (5 mL) at room temperature. After 16 hours volatiles were removed, the residue was triturated with DCM (50 mL) and filtered. The filtrate was extracted with methanol and solvent removed to give 420 mg 1035 as HCl salt.

### Example 29: Synthesis of Compounds Derived from Common Int.-2

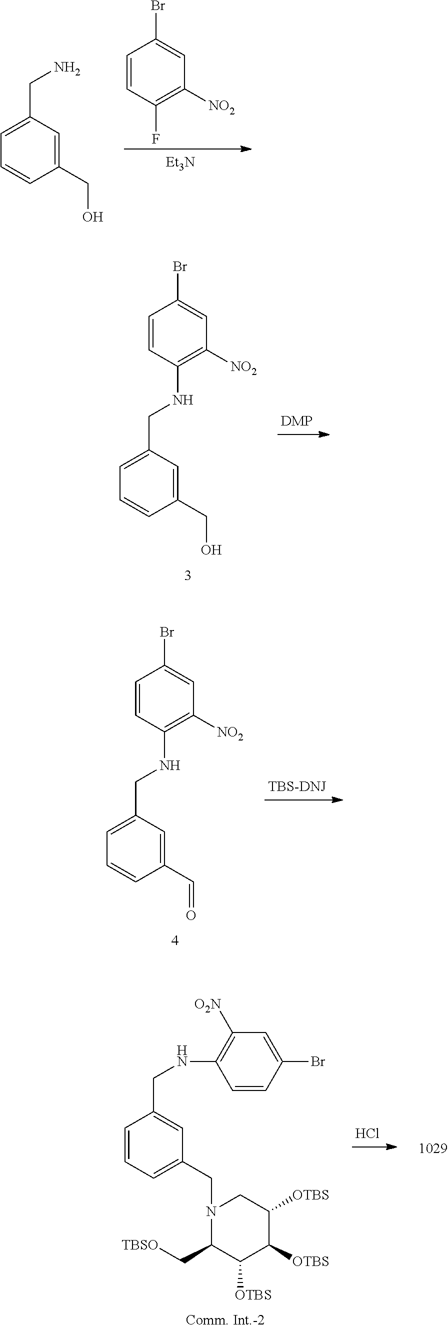

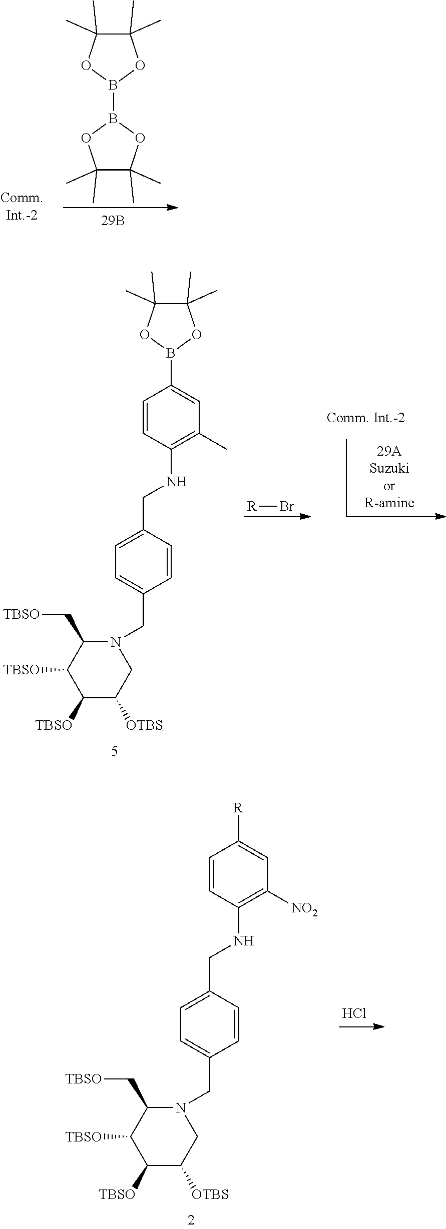

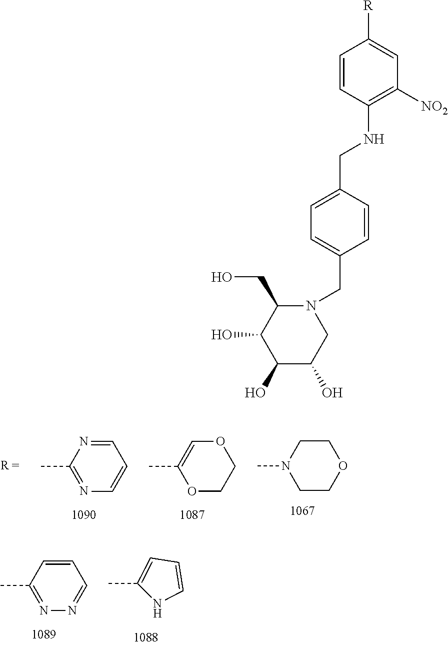

(3-(aminomethyl)phenyl)methanol (prepared as shown for para isomer in Example 13, 8.5 g), 1,4-dioxane (170 mL), Et₃N (3.0 eq), 1-fluoro-4-bromo-2-nitrobenzene (1.0 eq) mixed at room temperature then at 100° C. for 16 hours. Reaction volatiles were removed and the mass diluted with EtOAc and water. Separated the organic layer and dried over anhydrous Na₂SO₄ then concentrated and purified on silica [30% EtOAc-hexane] to afford 18 g of 3.

Mixed 3 (18.0 g) in DCM (360 mL) at 0° C., added DMP (1.5 eq), increased to room temperature for 1 hour. Reaction was diluted with water and DCM. Separated the organic layer and dried over anhydrous Na₂SO₄ then concentrated to afford 18 g of 4.

Mixed 4 (18 g), MeOH (360 mL), DCM (50 mL), TBS-DNJ (1.0 eq), AcOH (0.5 mL) and NaCNBH₃ (1.5 eq) at room temperature for 16 hours. Reaction volatiles were removed and diluted with EtOAc and water. The organic layer was dried over anhydrous Na₂SO₄, concentrated, and purified on silica [30% EtOAc in hexane] to afford 30 g of common Int-2.

For scheme 29B, in one example common Int. 2 (10.0 g), bis(pinacolato) diboron (1.5 eq), and KOAc (3.0 eq) in 1,4-dioxane (200 mL) were degassed with N₂ 10 minutes, Pd(dppf)Cl₂ (0.1 eq) added then increased to 100° C. After 16 hours reaction volatiles were removed and diluted with EtOAc and water. Separated the organic layer and dried over anhydrous Na₂SO₄ then concentrated to afford 11.0 g of boronate 5.

Preparation of 1029 (2R,3R,4R,5S)-1-[(3-{[(4-bromo-2-nitrophenyl)-amino]methyl}phenyl)methyl]-2-(hydroxymethyl)piperidine-3,4,5-triol: Deprotection was carried out prior to the coupling. Mixed common Int-2 (5.0 g), MeOH (75 mL), DCM (5 mL), 4.0 M HCl in 1,4-dioxane (50 mL) at 0° C. then increased to room temperature. After 16 hours solvent was removed and reaction mass basified with NaHCO₃. The mix was diluted with water (100 mL), extracted with EtOAc (3×150 mL), combined organic layer was dried over anhydrous Na₂SO₄, filtered, concentrated to afford 2.5 g 1029.

Preparation of 1087 (2R,3R,4R,5S)-1-{[3-({[4-(5,6-dihydro-1,4-dioxin-2-yl)-2-nitro-phenyl]amino}methyl)phenyl]methyl}-2-(hydroxymethyl)piperidine-3,4,5-triol (29A): Degassed 1029 (2.3 g), 1,4-dioxane:H₂O (3:1, 120 mL), 2-(5,6-dihydro-1,4-dioxin-2-yl)-boronate (1.5 eq), Cs₂CO₃ (3.0 eq) 15 minutes with N₂, mixed Pd(dppf)Cl₂ (0.1 eq) at room temperature then increased to 80° C. After 16 hours the reaction mass was diluted with water (40 mL) and extracted with EtOAc (3×50 mL). The combined organic layer was dried over anhydrous Na₂SO₄ filtered, concentrated, and preparative HPLC purification using normal phase column provided 800 mg which was washed with water and dried under vacuum to afford 570 mg of 1087.

Preparation of 1089 (2R,3R,4R,5S)-2-(hydroxymethyl)-1-{[3-({[2-nitro-4-(pyridazin-3-yl)phenyl]amino}methyl)phenyl]methyl}piperidine-3,4,5-triol (29B): Boronate 5 (5.0 g), toluene:ethanol:water (1:1:1, 120 mL), 3-bromopyridazine (1.0 eq), Na₂CO₃ (3.0 eq), degassed 15 minutes with N₂, then Pd(dppf)Cl₂ (0.1 eq) addition at room temperature then increased to 80° C. After 16 hours reaction volatiles were removed and diluted with EtOAc and water. Separated the organic layer and dried over anhydrous Na₂SO₄ then concentrated, and purified on silica [eluent 10% EtOAc-hexane] to afford 3.2 g of 4.

4 (3.2 g), MeOH (20 mL), DCM (20 mL), mixed at 0° C., added 4.0 M HCl in 1,4-dioxane (15 mL) at 0° C. then increased to room temperature. After 16 hours reaction volatiles were removed, and triturated with Et₂O to afford solid material which was dissolved in water (20 mL), neutralized with saturated NaHCO₃ at 0° C. and the precipitate was filtered to afford 1.3 g of 1089 as yellow solid.

Preparation of 1090 (2R,3R,4R,5S)-2-(hydroxymethyl)-1-{[3-({[2-nitro-4-(pyrimidin-2-yl)phenyl]amino}methyl)phenyl]methyl}piperidine-3,4,5-triol (29B): Boronate 5 (5.0 g), toluene:ethanol:water (1:1:1, 120 mL), 2-bromopyrimidine (1.0 eq), Na₂CO₃ (3.0 eq), degassed 15 minutes with N₂, Pd(dppf)Cl₂ (0.1 eq) added at room temperature then increased to 80° C. After 16 hours reaction volatiles were removed and diluted with EtOAc and water. Separated the organic layer and dried over anhydrous Na₂SO₄ then concentrated and purified on silica [10% EtOAc/hexane] to afford 3.2 g of 2.

2 (3.2 g), MeOH (20 mL), DCM (20 mL) mixed at 0° C., then 4.0 MHCl in 1,4-dioxane (15 mL) added at 0° C. then increased to room temperature. After 16 hours reaction volatiles were removed. The material obtained was washed with Et₂O to afford solid material which was dissolved in water (20 mL), neutralized with saturated NaHCO₃ at 0° C. and the precipitate was filtered to afford 1.2 g of 1090 as yellow solid.

Preparation of 1088 (2R,3R,4R,5S)-2-(hydroxymethyl)-1-{[3-({[2-nitro-4-(1H-pyrrol-2-yl)phenyl]amino}methyl)phenyl]methyl}piperidine-3,4,5-triol (29A): Common Int-2 (5.0 g), DME:H₂O (3:1, 100 mL), 1-Boc-pyrrole-2-boronate (1.5 eq), Cs₂CO₃ (1.5 eq), degassed for 15 minutes with N₂, then added Pd(dppf)Cl₂ (0.1 eq) at room temperature then increased to 80° C. After 16 hours reaction mass was diluted with water (50 mL) and extracted with EtOAc (2×100 mL). The combined organic layer was dried over anhydrous Na₂SO₄, filtered, concentrated, and purified on silica [5% EtOAc/hexane] to afford 3.5 g of 2 as orange red thick syrup.

2 (2.0 g), MeOH:DCM (30 mL, 3:1), 4.0 M HCl in 1,4-dioxane (20 mL) mixed at 0° C. then room temperature for 8 hours. The solvent was removed, reaction mass was diluted with water, basified with saturated aq. NaHCO₃ and extracted with EtOAc (3×150 mL). The combined organic layers were dried over anhydrous Na₂SO₄ filtered, concentrated, and purified by preparative HPLC using normal phase column to provide 200 mg of 1088.

Preparation of 1067 (2R,3R,4R,5S)-2-(hydroxymethyl)-1-{[3-({[4-(morpholin-4-yl)-2-nitrophenyl]amino}methyl)phenyl]methyl}piperidine-3,4,5-triol (29A): Common Int-2 (5 g), 1,4-dioxane (50 mL), Cs₂CO₃ (2.0 eq), Pd₂(dba)₃ (0.2 eq), xantphos (0.2 eq) was degassed 15 minutes with N₂, added morpholine (3.0 eq) at room temperature then increased to 80° C. After 16 hours reaction volatiles were removed. The residue was diluted with EtOAc and water. The organic layer was dried over anhydrous Na₂SO₄, concentrated, and purified on silica [5-10% EtOAc/hexane] to afford 2.2 g of 2.

2 (1.7 g), DCM (20 mL), 4.0 M HCl in 1, 4-dioxane (4 mL) at 0° C. then increased to room temperature. After 16 hours organic volatiles were removed, the material was triturated with DCM (2×50 mL) for 10 minutes at room temperature and filtered to afford 400 mg of red 1067 HCl salt.

### Example 30: Synthesis of Compound 1092 ((2R,3R,4R,5S)-2-(hydroxymethyl)-1-({4-[({4-[(1,2-oxazolidin-2-yl)methyl]phenyl}amino)methyl]phenyl}methyl)piperidine-3,4,5-triol)

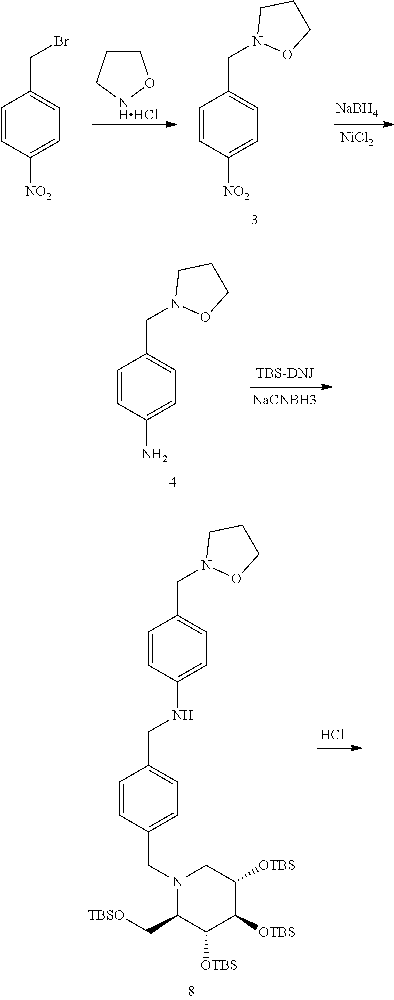

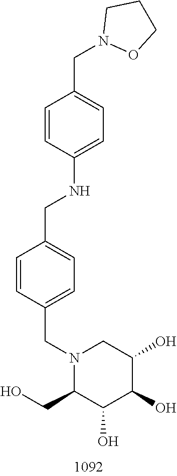 [SMILES: OC[C@@H]1[C@@H](O)[C@H](O)[C@@H](O)CN1Cc1ccc(CNc2ccc(CN3CCCO3)cc2)cc1]

1-(bromomethyl)-4-nitrobenzene (10 g), ACN (100 mL), isoxazolidine (1.5 eq) mixed at room temperature, K₂CO₃ (3 eq) added at room temperature, increased to 80° C. After 16 hours reaction was concentrated, residue was dissolved in water and extracted with EtOAc (2×50 mL). The organic layer was dried over anhydrous Na₂SO₄, filtered, concentrated, and purified on silica [5% EtOAc/hexane] to afford 6.0 g of 3.

3 (1 g), DCM (30 mL), MeOH (10 mL), NiCl₂·6H₂O (1 eq), NaBH₄ (5 eq) mixed at −20° C. After 10 minutes the reaction was quenched with ice-cold water and extracted with DCM (2×30 mL). The organic layer was dried over anhydrous Na₂SO₄, filtered, concentrated, and purified on silica [eluting with 70% EtOAc/hexane] to afford 500 mg of 4.

TBS-DNJ (1.5 g), 4 (1.0 eq), MeOH (40 mL), AcOH (cat.), and NaCNBH₃ (1.5 eq) were mixed at room temperature. After 30 hours the reaction was concentrated, the residue dissolved in water and extracted with EtOAc (50 mL), the organic layer dried over anhydrous Na₂SO₄, filtered, concentrated, and purified on silica [20% EtOAc in hexane] to afford 1.0 g of 8.

8 (500 mg), DCM (15 mL), 4.0 MHCl in 1,4-dioxane (5 mL) mixed at 0° C. then increased to room temperature. After 2 hours the reaction volatiles were removed. Two batches were combined and purified by preparative HPLC to afford 130 mg of 1092 as colorless thick syrup.

### Example 31: Synthesis of Compound 1068 ((2S,3S,4S,5R)-2-(hydroxymethyl)-1-{[4-({[4-(morpholin-4-yl)phenyl]amino}methyl)phenyl]methyl}piperidine-3,4,5-triol)

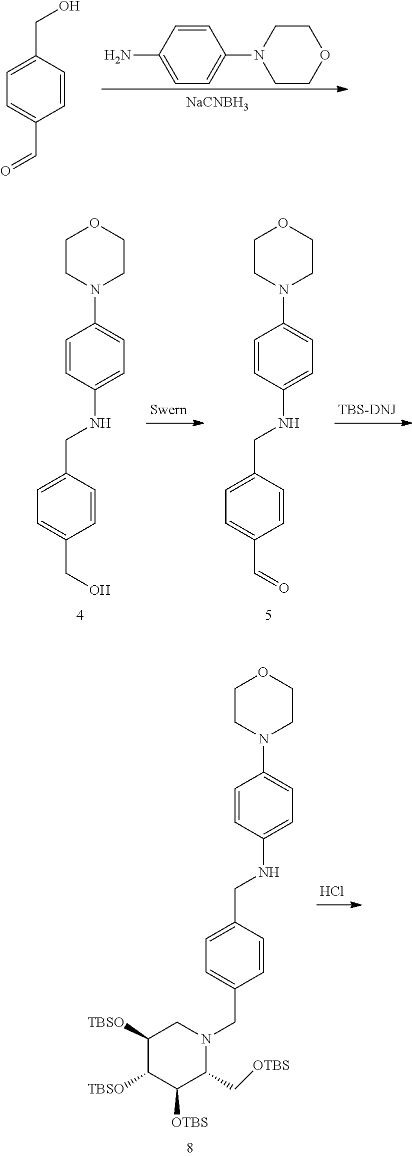

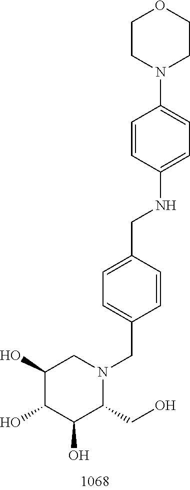 [SMILES: OC[C@@H]1[C@@H](O)[C@H](O)[C@@H](O)CN1Cc1ccc(CNc2ccc(N3CCOCC3)cc2)cc1]

4-(Hydroxymethyl)-benzaldehyde (5 g), MeOH/DCM (1:1, 150 mL), 4-morpholino-aniline (Example 33, 1.0 eq), NaCNBH₃ (1.5 eq), AcOH (cat.,) addition at 0° C. then increased to room temperature. After 16 hours reaction was concentrated, residue was dissolved in water and extracted with EtOAc (2×200 mL), the organic layer was dried over Na₂SO₄, filtered, concentrated, and purified on silica [1.5% MeOH in DCM] to afford 5 g of 4.

(COCl)₂ (3 eq), −78° C., DCM (250 mL), DMSO (4 eq), 4 (5 g) mixed for 1.5 hours then TEA (5 eq) added and increased to room temperature for 30 minutes. Water was added and extracted with DCM (100 mL). The organic layer was washed with water and dried over Na₂SO₄, filtered, concentrated to afford 4 g of 5.

5 (4 g), TBS-DNJ (0.8 eq), MeOH/DCM (1:1, 1 L), AcOH (2 mL), NaCNBH₃ (1.5 eq), addition at 0° C. then increased to room temperature. After 16 hours the reaction was concentrated. The residue was dissolved in water and extracted with EtOAc (2×400 mL). The organic layer was dried over Na₂SO₄, filtered, concentrated, and purified on silica using with 1.5% EtOAc in hexane to afford 3 g of 8.

8 (3 g), DCM (50 mL), 4.0 MHCl in dioxane (30 mL) mixed at 0° C. then room temperature for 16 hours. Reaction volatiles were removed, and residue triturated with DCM followed by drying under vacuum. Solid was taken in MeOH (15 vol, 15 mL), heated to reflux for 15 minutes, cooled to room temperature and filtered to obtain 750 mg of 1068 as HCl salt.

### Example 32: Synthesis of Compound 1066 ((2R,3R,4R,5S)-1-{[3-({[4-(5,6-dihydro-1,4-dioxin-2-yl)phenyl]amino}methyl)phenyl]methyl}-2-(hydroxymethyl)piperidine-3,4,5-triol)

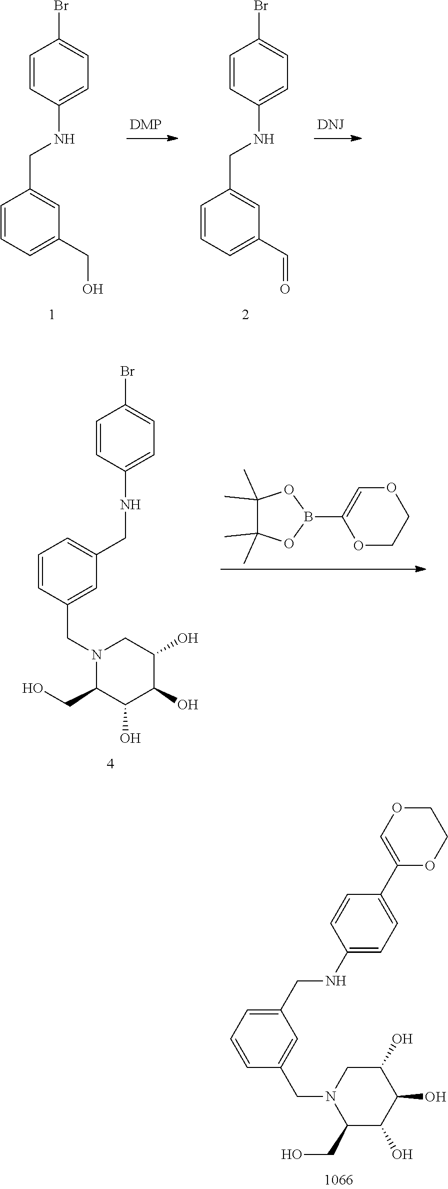 [SMILES: OCc1cccc(CNc2ccc(Br)cc2)c1 ; O=Cc1cccc(CNc2ccc(Br)cc2)c1 ; OC[C@@H]1[C@@H](O)[C@H](O)[C@@H](O)CN1Cc1cccc(CNc2ccc(Br)cc2)c1 ; CC1(C)OB(C2=COCCO2)OC1(C)C ; OC[C@@H]1[C@@H](O)[C@H](O)[C@@H](O)CN1Cc1cccc(CNc2ccc(C3=COCCO3)cc2)c1]

1 (Example 14, 1 g), DCM (100 mL), DMP (1 eq) added at 0° C. and stirred 15 minutes. Reaction was diluted with water and extracted with DCM. Organic layer was dried over anhydrous Na₂SO₄ and concentrated to afford 1 g of 2.

To a stirred solution of 2 (1.0 g) in MeOH was added DNJ (562 mg, 1.0 eq) and AcOH (cat.) under nitrogen atmosphere at room temperature. The reaction flask was cooled to 0° C. then added NaCNBH₃ and stirred at room temperature for 16 hours. After 16 hours the reaction was concentrated, the residue was dissolved in water and extracted with Et₂O (300 mL), the aqueous layer was super-saturated with NaCl and extracted with EtOAc (3×100 mL). The organic layer was washed with water, dried over anhydrous Na₂SO₄, concentrated to afford 4.

4 (700 mg), 2-(5,6-dihydro-1,4-dioxin-2-yl)-boronate (2.5 eq), Cs₂CO₃ (4.0 eq), 1,4-dioxane:water (3:1, 30 mL), degassed 15 minutes with N₂, Pd(dppf)Cl₂ (0.2 eq) mixed and bought to 100° C. After 16 hours the reaction was concentrated. The residue was dissolved in water and extracted with EtOAc (3×250 mL). The organic layer was washed with water and dried over anhydrous Na₂SO₄, concentrated, and triturated with Et₂O then EtOAc. After preparative HPLC purification 150 mg of 1066 was obtained.

### Example 33: Synthesis of Compound 1081 ((2R,3R,4R,5S)-2-(hydroxymethyl)-1-{[3-({[4-(morpholin-4-yl)phenyl]amino}methyl)phenyl]methyl}piperidine-3,4,5-triol)

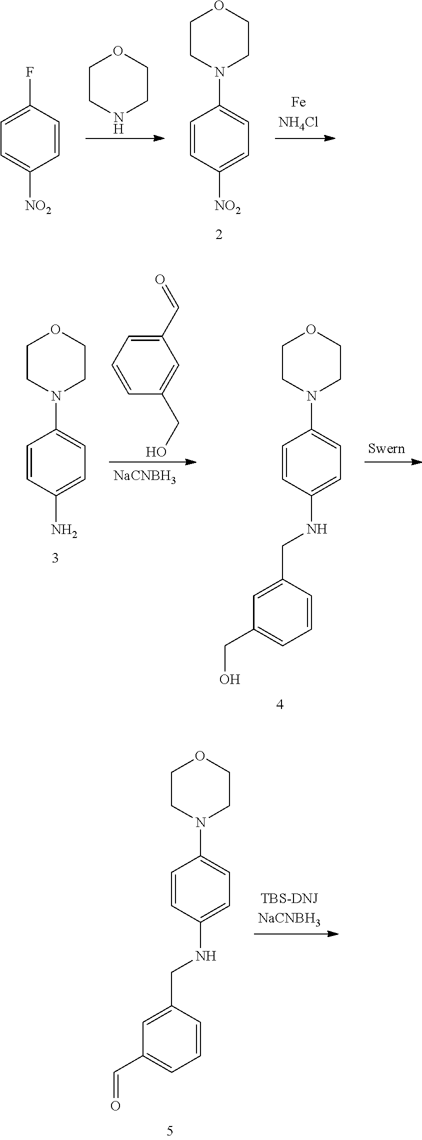

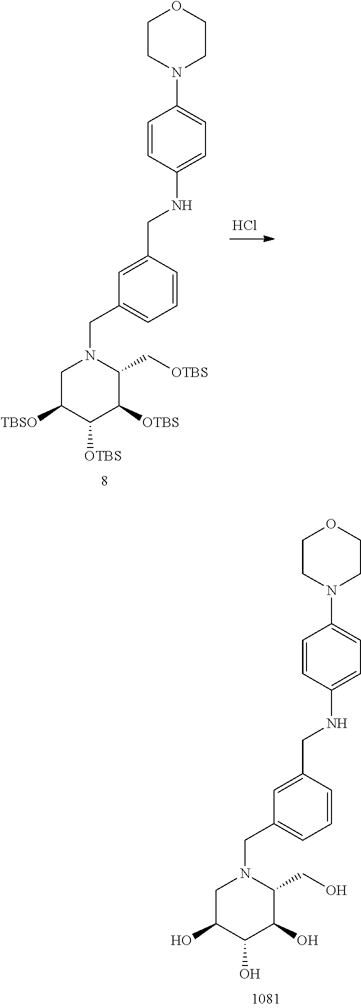 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1Cc1cccc(CNc2ccc(N3CCOCC3)cc2)c1 ; Cl ; OC[C@@H]1[C@@H](O)[C@H](O)[C@@H](O)CN1Cc1cccc(CNc2ccc(N3CCOCC3)cc2)c1]

Mixed 4-fluoro-nitrobenzene (15 g), morpholine (1.5 eq), 1,4-dioxane (150 mL) at room temperature, increased to 100° C. After 16 hours concentrated the reaction dissolved, residue in water (1 L), extracted with EtOAc (2×1 L). Washed the organic layer with water, dried over anhydrous Na₂SO₄, concentrated, and triturated with hexane to afford 2 (15 g).

Mixed 2 (15 g), Fe (5 eq), NH₄Cl (5 eq), water (100 mL) at room temperature, increased to 70° C. for 6 hours. Reaction was filtered over CELITE, filtrate was extracted with EtOAc (2×1 L). The organic layer was dried over anhydrous Na₂SO₄ and concentrated to afford 12 g of 3.

3-(Hydroxymethyl)-benzaldehyde (3 g), MeOH (40 mL), 3 (1.0 eq) mixed at 0° C., AcOH (cat.) and NaCNBH₃ (1.5 eq) added at 0° C. then reacted at room temperature for 16 hours. The reaction was concentrated and the residue dissolved in water (500 mL) and extracted with EtOAc (2×500 mL). The organic layer was washed with water and dried over anhydrous Na₂SO₄, concentrated, and purified on silica [1.2% MeOH in DCM] to obtain 1.5 g of 4.

(COCl)₂ (3.0 eq), DCM (3 mL) cooled to −78° C., DMSO (4 eq) added for 30 minutes, 4 (1.3 g) added for 2 hours Et₃N (5 eq) added and warmed to room temperature over 30 minutes. After 3 hours the reaction was diluted with water and extracted with DCM (2×50 mL). The solvent was dried over Na₂SO₄, filtered, concentrated to afford 1.3 g of 5.

5 (1.3 g), MeOH:DCM (1:1, 400 mL), TBS-DNJ (1.0 eq) mixed at 0° C., AcOH (cat.), NaCNBH₃ (1.5 eq) added at 0° C., then warmed to room temperature. After 16 hours the reaction was concentrated. The residue was dissolved in water (250 mL) and extracted with EtOAc (2×200 mL). The organic layer was washed with water and dried over anhydrous Na₂SO₄, concentrated to afford 8.

8 (3.0 g), DCM (30.0 mL), 4.0 MHCl in 1,4-dioxane (30 mL) added at 0° C. then increased to room temperature. After 16 hours the reaction was concentrated. Material was triturated with DCM and dried to afford 1.13 g of 1081.

### Example 34: Synthesis of Compound 1098 ((2R,3R,4R,5S)-2-(hydroxymethyl)-1-([{4-[([{2-nitro-4-[(1,2-oxazolidin-2-yl)methyl]phenyl}amino)methyl]phenyl}methyl)piperidine-3,4,5-triol)

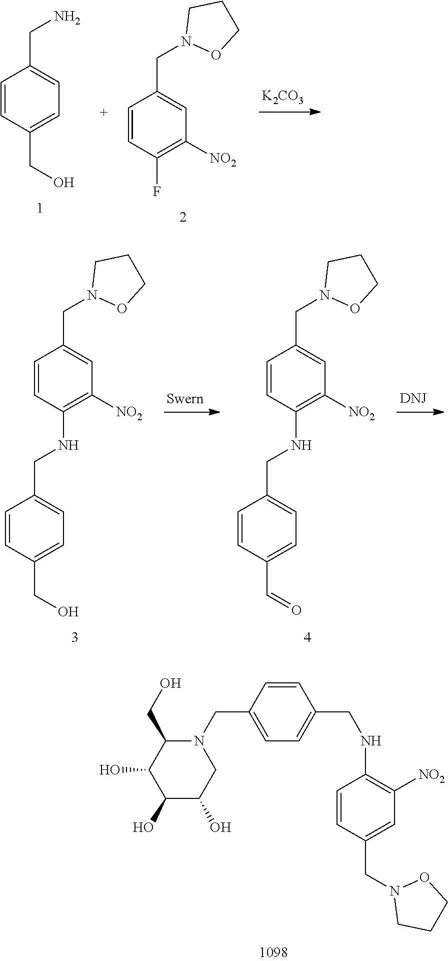

2 (Example 16, 1.7 g), ACN (40 mL), K₂CO₃ (4.0 eq), 1 (Example 1, 2.0 eq) mixed at room temperature then refluxed 16 hours. Solvent was removed. Residue was diluted with water, extracted with EtOAc (2×40 mL). Combined organic layers were washed with brine solution, dried over anhydrous Na₂SO₄, concentrated, and purified on silica [30% EtOAc-hexane to 100% EtOAc] to afford 1.8 g of 3.

(COCl)₂ (3.0 eq), DCM (20 mL) cooled to −78° C., DMSO (4 eq) added for 30 minutes, 3 (1 g) added for 2 hours TEA (5 eq) added and increased to room temperature for 30 minutes. After 3 hours the reaction was diluted with water and extracted with DCM (2×40 mL). The organic layer was dried over Na₂SO₄, filtered, concentrated to afford 1 g of 4.

4 (900 mg), MeOH:DCM (100 mL, 1:1), DNJ (1.0 eq) mixed at room temperature, AcOH (cat.), NaCNBH₃ (1.5 eq) added at room temperature for 72 hours. The reaction was concentrated and the residue diluted with water and extracted with 10% MeOH:DCM (4×40 mL). Combined organic layers were dried over anhydrous Na₂SO₄, concentrated, and triturated with Et₂O (3×30 mL) and EtOAc (3×20 mL) to afford 550 mg of yellow solid. Preparative HPLC purification yielded 300 mg 1098 as yellow solid.

### Example 35: Synthesis of Compound 1103 ((2R,3R,4R,5S)-1-[2-(3-{2-[(4-azido-2-nitrophenyl)amino]ethyl}bicyclo[1.1.1]pentan-1-yl)ethyl]-2-(hydroxymethyl)piperidine-3,4,5-triol)

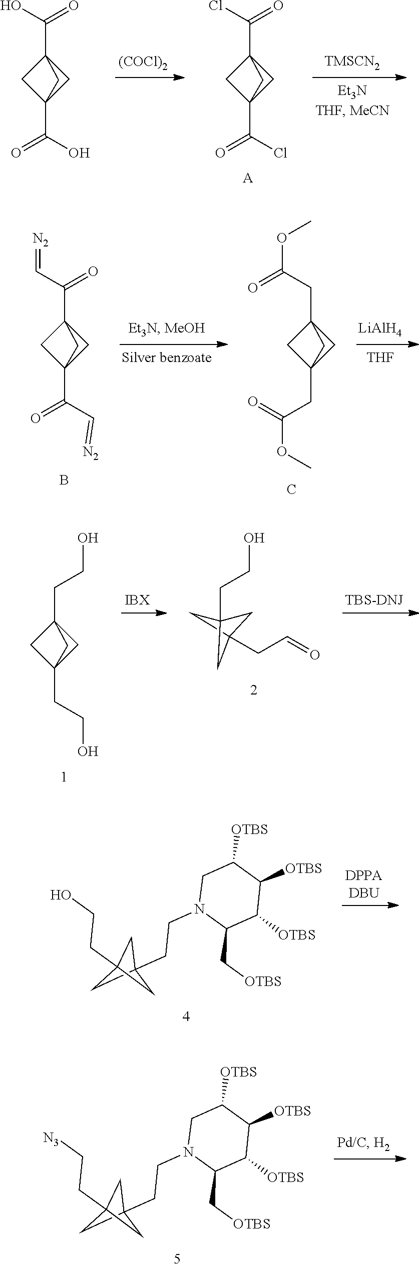

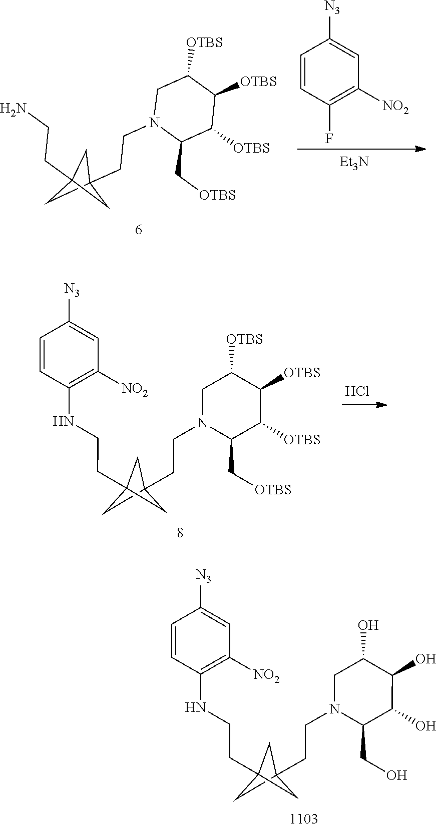 [SMILES: "Et3N" ; CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCC12CC(CCN)(C1)C2 ; [N-]=[N+]=Nc1ccc(F)c([N+](=O)[O-])c1 ; CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCC12CC(CCNc3ccc(N=[N+]=[N-])cc3[N+](=O)[O-])(C1)C2 ; Cl ; [N-]=[N+]=Nc1ccc(NCCC23CC(CCN4C[C@H](O)[C@@H](O)[C@H](O)[C@H]4CO)(C2)C3)c([N+](=O)[O-])c1]

To an ice-water cooled solution of bicyclo[1.1.1]pentane-1,3-dicarboxylic acid (39 g) in DCM (300 mL) was added DMF (˜5 drops) followed by (COCl)₂ (66 g). The reaction was stirred at 0° C. for 30 minutes and then at room temperature overnight. The solvent was evaporated to give bicyclo[1.1.1]pentane-1,3-dicarbonyl dichloride (A).

Ttrimethylsilyldiazomethane (2 M, 313 mL) was added dropwise to an ice-water cooled solution of A, Et₃N (63.125 g) in a 1:1 solution of ACN:THF (200 mL). The mixture was stirred at 0° C. for 1 hour then overnight at room temperature. Solvent was removed, the residue was redissolved in EtOAc (500 mL), washed with brine (200 mL), dried over Na₂SO₄, concentrated in vacuo, and the residue purified on silica to give B (35.7 g).

To a solution of B (35.7 g) in MeOH (500 mL) was added TEA (88.375 g), followed by silver benzoate (14.8 g) at 0° C. The mixture was stirred at room temperature overnight. MeOH was removed by evaporation and the residue dissolved in EtOAc (500 mL) and washed with NaHCO₃ (200 mL), citric acid (2 M, 200 mL), brine (200 mL), dried over MgSO4, concentrated in vacuo and purified on silica to afford C (21.23 g).

To a solution of LiAlH₄ (15.2 g) in THF (1000 mL) added C (21.23 g) in THF (100 mL) maintaining at 0° C. The resulting mixture was stirred at room temperature overnight, quenched with water (15.2 mL) and 15% NaOH aq. (15.2 mL) and purified on silica to afford 1 (13.5 g).

1 (1.5 g), IBX (0.5 eq), AcOH (1.5 eq), ACN (55 mL) were mixed at room temperature for 24 hours. The reaction was filtered through CELITE bed and washed with EtOAc (20 mL). The filtrate was concentrated. The residue was dissolved in EtOAc (20 mL) and washed with water and dried over anhydrous Na₂SO₄, filtered, concentrated to afford 2.

Mixed 2 (1.5 g), TBS-DNJ (1 eq), MeOH (20 mL), AcOH (0.05 mL), NaCNBH₃ (1.5 eq) 16 hours at room temperature. The reaction was concentrated and the residue dissolved in water and extracted with EtOAc (20 mL). The organic layer was dried over anhydrous Na₂SO₄, filtered, concentrated, and purified on silica [2% EtOAc in hexane] to afford 800 mg of 4.

4 (800 mg), toluene (20 mL), DBU (3 eq), DPPA (3 eq) were added at room temperature then increased to 100° C. After 16 hours reaction volatiles were removed, residue was dissolved in water, extracted with EtOAc (50 mL), the organic layer was dried over anhydrous Na₂SO₄, filtered, concentrated, and purified on silica [2% EtOAc-hexane] to afford 700 mg of 5.

5 (700 mg), 10% Pd/C (50% wet, 400 mg), EtOH (5 mL) were reacted under modest H₂ pressure (balloon) at room temperature for 1 hour. The reaction mixture was filtered through CELITE bed and washed with EtOAc (100 mL). The filtrate was concentrated to afford 600 mg of 6.

6 (600 mg), FNAB (1 eq), dioxane (20 mL), Et₃N (5 eq) reacted at 90° C. for 16 hours. Volatiles were removed and residue purified on silica [2% EtOAc-hexane] to afford 500 mg of 8.

8 (350 mg), MeOH (5 mL), DCM (5 mL) were mixed at 0° C., then 4.0 M HCl in dioxane (1.6 mL) added at 0° C. and increased to room temperature for 1 hour. Reaction volatiles were distilled, and dissolved in MeOH and basified with aq. NH₃ solution, then volatiles were evaporated. After lyophilization 16 mg of 1103 was obtained as red solid.

### Example 36: Synthesis of Compound 1107 ((2R,3R,4R,5S)-2-(hydroxymethyl)-1-{2-[(1r,4r)-4-{2-[(4-azido-2-nitrophenyl)amino]ethyl}cyclohexyl]ethyl}piperidine-3,4,5-triol)

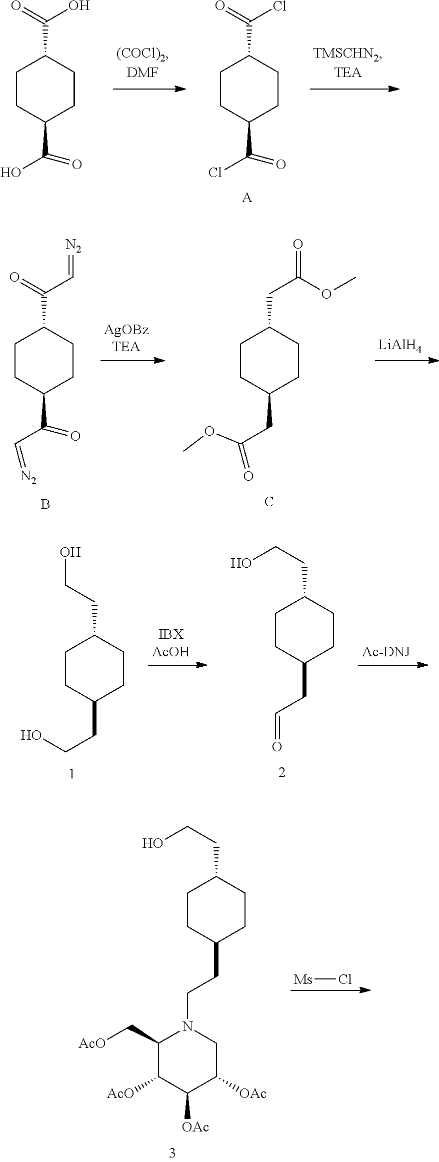

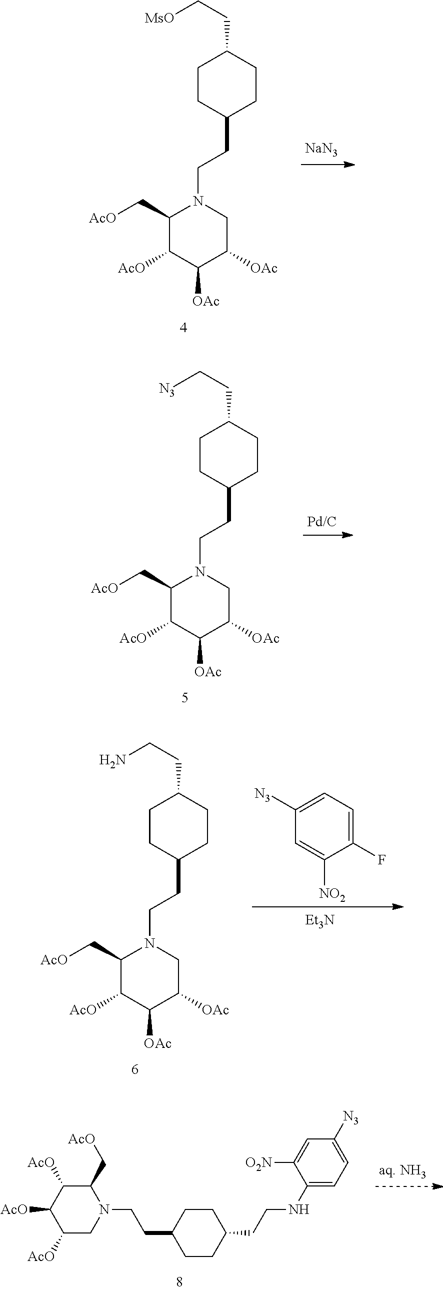

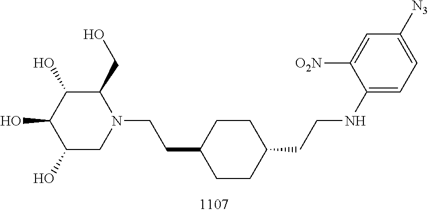

(1r,4r)-Cyclohexane-1,4-dicarbonic acid (44 g), DMF (372 mg) were dissolved in DCM (1000 mL) then added oxalyl chloride (70 g) at 0° C. The mixture was stirred at room temperature for 4 hours then concentrated to afford the dichloride A.

To a solution of (trimethylsilyl)diazomethane (318 mL), TEA (64 g), THE (1000 mL) and ACN (1000 mL) added A dropwise at 0° C., stirred at room temperature overnight. The mixture was concentrated, diluted with DCM (1000 mL), washed with brine (200 mL), dried over Na₂SO₄, concentrated in vacuo. The material was purified on silica and a gradient of 0% to 50% EtOAc in petroleum ether to afford B (24.43 g) as colorless oil.

To a solution of B (24.43 g) in MeOH (500 mL) added TEA (89 g), followed by silver benzoate (9.4 g) at 0° C. The mixture was stirred at room temperature overnight then concentrated in vacuo and purified on silica and a gradient of 0% to 80% EtOAc in petroleum ether to afford C as colorless oil (19.32 g).

To a solution of LiAlH₄ (9.6 g) in THE (1000 mL) added C (19.32 g) in THE (100 mL) maintaining at 0° C., stirred at room temperature overnight, quenched with 15% NaOH aq. (20 mL), washed with brine (100 mL), dried over Na₂SO₄, concentrated, and purified on silica using 70% EtOAc in petroleum ether to afford 1 (14.4 g) as white solid.

1 (500 mg), ACN (15 mL) mixed at 0° C., added IBX (1.5 eq), AcOH (1.2 eq) at 0° C., then increased to room temperature. After 16 hours reaction was diluted with EtOAc, filtered through pad of CELITE. The filtrate was washed with water. The organic layer was dried over anhydrous Na₂SO₄ then concentrated, and purified on silica with 30% EtOAc:hexane to afford 240 mg of 2 (also recovered 240 mg of 1).

2 (240 mg), MeOH (10 mL), Ac-DNJ (1.0 eq) mixed at 0° C., then AcOH (cat.), NaCNBH₃ (1.5 eq), addition at 0° C., increased to then room temperature. After 16 hours reaction was diluted with water and extracted with EtOAc (2×50 mL). The organic layer was washed with sat. citric acid, dried over anhydrous Na₂SO₄, concentrated, and purified on silica [30% EtOAc/hexane as eluent] to afford 260 mg of 3.

3 (260 mg), DCM (10 mL) at 0° C., added Et₃N (5.0 eq), MsCl (2.0 eq) at 0° C. for 15 minutes. Reaction was diluted with DCM and water; separated the organic layer, dried over anhydrous Na₂SO₄ concentrated to afford 280 mg of 4.

4 (280 mg), DMF (15 mL), NaN₃ (5.0 eq) added at room temperature then increased to 70° C. After 1 hour reaction mass was diluted with EtOAc and water; separated the organic layer, dried over anhydrous Na₂SO₄, filtered, concentrated, and purified on silica [25% EtOAc/hexane] to afford 150 mg of 5.

5 (150 mg), MeOH (10 mL), 10% Pd/C (50% wet, 100 mg), under mild H₂ pressure at room temperature. After 2 hours reaction mass filtered through pad of CELITE. The filtrate was concentrated to afford 140 mg of 6.

Mixed 6 (140 mg), 1,4-dioxane (10 mL), FNAB (1.0 eq), Et₃N (3.0 eq) at room temperature then increased to 100° C. After 16 hours reaction was diluted with EtOAc (100 mL) and washed with water (2×100 mL). The organic layer was dried over anhydrous Na₂SO₄, concentrated, and purified on silica (30% EtOAc in hexane) to afford 70 mg of 8.

8 (70 mg), MeOH (4 mL), 0° C., aq·NH₃ (4 mL), addition at 0° C. then raised to room temperature. After 24 hours, the reaction volatiles were removed. Preparative HPLC purification gave 19 mg of 1107 and further purification by chiral HPLC afforded 4.0 mg of 1107.

### Example 37. Synthesis of Compound 1105 ((2R,3R,4R,5S)-1-({4-[(4-{[(4-azido-2-nitrophenyl)amino]methyl}phenyl)methyl]phenyl}methyl)-2-(hydroxymethyl)piperidine-3,4,5-triol)

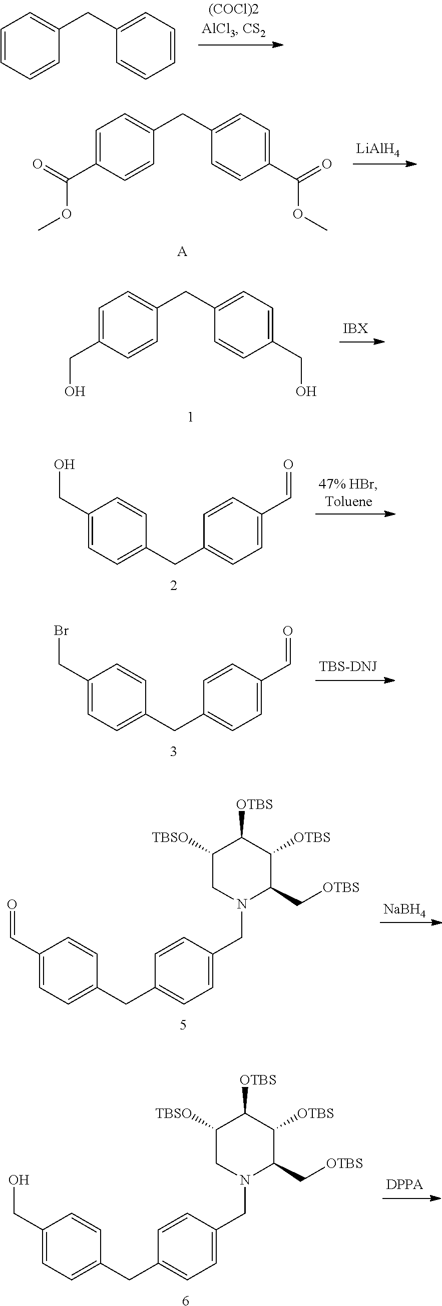

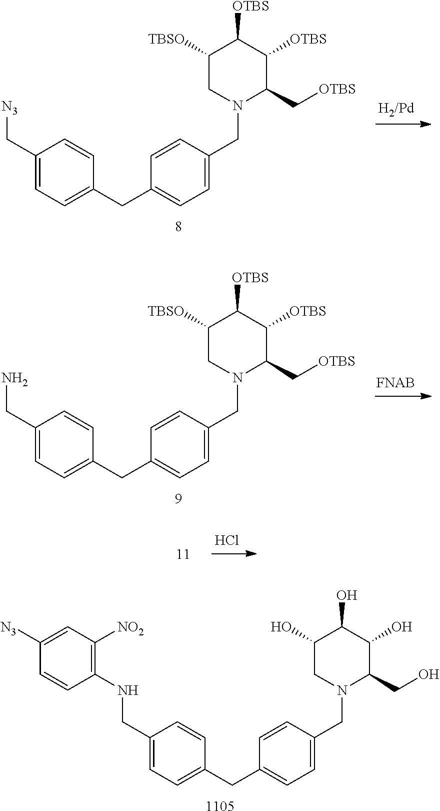

To a stirred solution of diphenylmethane (22 g) and oxalyl chloride (37.5 g) in dry carbon disulphide (200 mL) cooled to −5° C., anhydrous aluminum chloride (36 g) was added in small portions during 1 hour. The reaction mixture was stirred at 0° C. for 4 hours and at room temperature for 2 hours. Reaction mixture was cooled in ice, and dry methanol (200 mL) was added dropwise. The mixture was stirred at room temperature overnight. Ice cold hydrochloric acid was added and the mixture was extracted with Et₂O. The organic layer was successively washed with water, aqueous Na₂CO₃ and removal of solvent gave A (30 g).

To a solution of LiAlH₄ (5.7 g) in THF (1 L) at 0° C. added a solution of A in THE (50 mmol in 200 mL), stirred at room temperature overnight, quenched with water (5.7 mL) and 15% NaOH aqueous (5.7 mL), then purified on silica to afford 1 (10.26 g).

1 (1.3 g), ACN (50 mL) mixed at 0° C., IBX (1.2 eq), AcOH (1.2 eq) added then increased to room temperature. After 16 hours the reaction was diluted with EtOAc (30 mL) and filtered to remove solids. The filtrate was washed with water and dried over anhydrous Na₂SO₄, filtered, concentrated to afford 1.3 of 2. The obtained material was purified on silica [20% EtOAc in hexane] to afford 650 mg of 2.

2 (650 mg), toluene (30 mL), 47% aq. HBr (6.4 mL) heated to reflux and maintained 2 hours. The reaction was quenched with ice-cold water, extracted with EtOAc (50 mL), the organic layer dried over anhydrous Na₂SO₄, filtered, concentrated to afford 700 mg of 3.

3 (700 mg), TBS-DNJ (1 eq), K₂CO₃ (2 eq), ACN (5 mL) mixed and heated to 80° C. After 16 hours the reaction was cooled to room temperature and concentrated. The residue was dissolved in EtOAc (70 mL) washed with water and dried over anhydrous Na₂SO₄, filtered, concentrated, and purified on silica [2% EtOAc in hexane] to afford 800 mg of 5.

5 (800 mg), MeOH/THF (1:1, 30 mL), NaBH₄ (2 eq) mixed at room temperature for 2 hours. After 2 hours the residue was dissolved in EtOAc (30 mL) and washed with water and dried over Na₂SO₄, filtered, concentrated to afford 800 mg of product. The material was purified on silica [20% EtOAc in hexane] to afford 550 mg of 6.

6 (550 mg), toluene (15 mL), DPPA (3 eq), DBU (3 eq) heated to 100° C. After 16 hours the reaction was cooled to room temperature and concentrated, and purified on silica [20% EtOAc in hexane] to afford 300 mg of 8.

8 (300 mg), 10% Pd/C (50% wet, 50 mg), MeOH:EtOH (1:1, 20 mL) maintained under mild H₂ pressure (balloon) at room temperature 2 hours. The reaction mixture was filtered through CELITE bed and washed with MeOH. The filtrate was concentrated to afford 270 mg of 9.

9 (270 mg), FNAB (1 eq), 1,4-dioxane (15 mL), Et₃N (5 eq), heated to 100° C. After 16 hours reaction was cooled to room temperature and distilled, and purified on silica [2% EtOAc in hexane] to afford 150 mg of 11 (not shown in figure).

11 (150 mg), MeOH (5 mL), DCM (1 mL), mixed at 0° C., added 4.0 M HCl in dioxane (1.5 mL) and increased to room temperature for 1 hour. Reaction volatiles were removed. The reaction mass was dissolved in methanol and basified with aqueous ammonia solution and distilled. Preparative HPLC purification afforded 25 mg of 1105.

### Example 38: Synthesis of Compound 1106 ((2R,3R,4R,5S)-2-(hydroxymethyl)-1-{2-[(1s,4s)-4-{2-[(4-azido-2-nitrophenyl)amino]ethyl}cyclohexyl]ethyl}piperidine-3,4,5-triol)

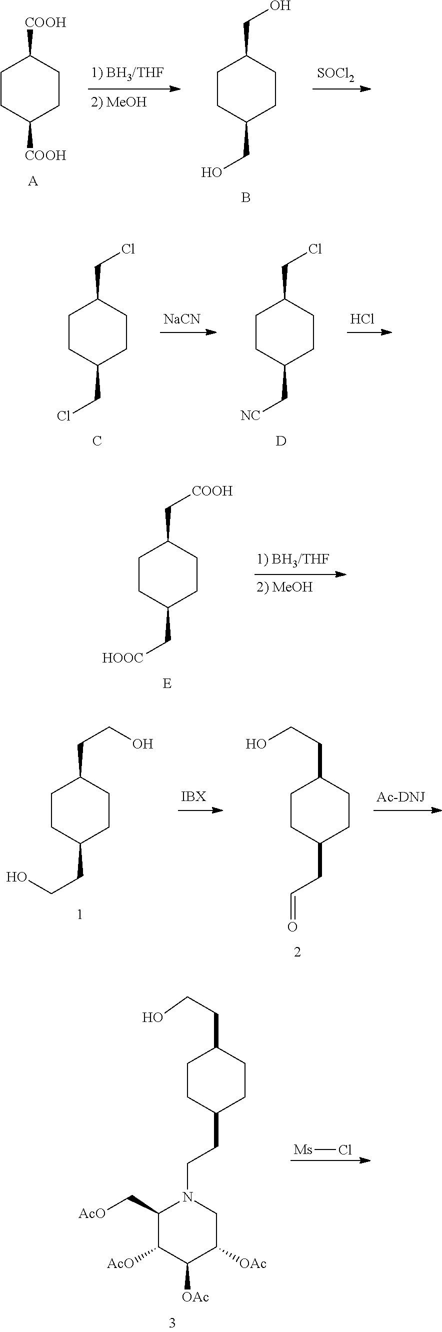

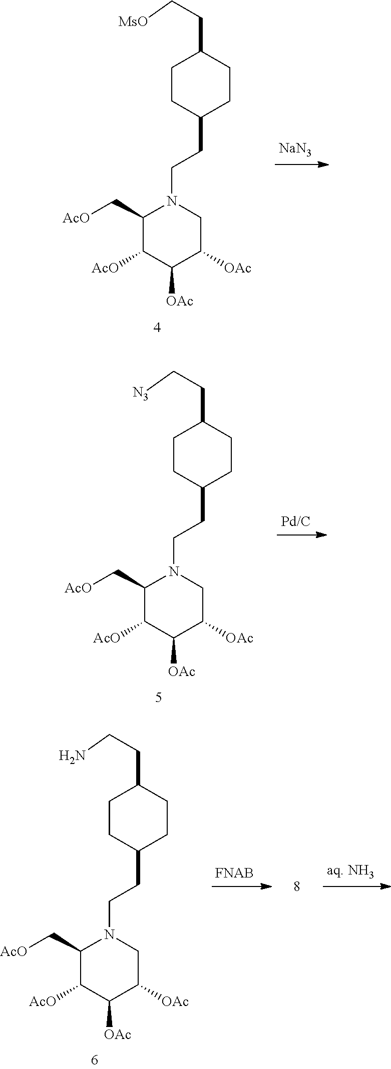

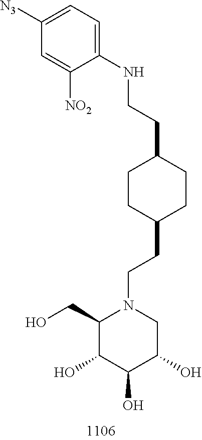

(1s,4s)-Cyclohexane-1,4-dicarboxcylic acid (120 g) in THE (150 mL), added BH₃ (2 M in THF, 1 L) at 0° C., stirred at room temperature for 16 hours added 100 mL MeOH at 0° C. Volatiles were evaporated and product purified on silica with 5% MeOH in DCM to afford B (72 g) as a colorless oil.

Thionyl chloride (108 mL) was treated with pyridine (100 mL) at 0° C., then B (72 g) was added at 0° C., warmed to 80° C., stirred 2 hours cooled to room temperature, 200 mL DCM added. The mixture was washed with water (200 mL), saturated NaHCO₃ solution (200 mL) and water (100 mL). The organic phase was dried over Na₂SO₄ and concentrated. The product was purified on silica with 100% petroleum ether to afford C (47.8 g) as a yellow oil.

To a solution of C (47.8 g) in DMF (100 mL) was added sodium cyanide (32.3 g). The mix was stirred at 100° C. for 12 hours then 500 mL DCM added. The mixture was washed with water (400 mL), saturated NaCl solution (200 mL) and water (200 mL). The organic phase was dried over Na₂SO₄ and concentrated. The product was purified on silica with 30% petroleum ether in EtOAc to afford D (28 g) as a yellow oil.

To a solution of D (28 g) in dioxane (20 mL) was added concentrated HCl (100 mL). The mixture stirred at 100° C. for 12 hours then concentrated to afford E (17.1 g) as a white solid.

To a solution of E (17.1 g) in THE (50 mL) added BH₃ (2M in THF, 128 mL) at 0° C. The mixture stirred at room temperature for 16 hours then 100 mL methanol was added at 0° C. The mixture was concentrated and product purified on silica with 5% of MeOH in DCM to afford 1 (11.5 g) as a yellowish oil.

1 (500 mg), ACN (15 mL), 0° C., IBX (1.5 eq), AcOH (1.2 eq) were mixed at 0° C., then at room temperature for 16 hours. Reaction mass was diluted with EtOAc, filtered through pad of CELITE. The filtrate was washed with water and organic layer dried over anhydrous Na₂SO₄ then concentrated, and purified on silica with 30% EtOAc in hexane to afford 230 mg of 2.

2 (230 mg), MeOH (10 mL), Ac-DNJ (1.0 eq), 0° C., AcOH (cat.), NaCNBH₃ (1.5 eq) were mixed at 0° C. then room temperature for 16 hours. Reaction was diluted with water and extracted with EtOAc (2×50 mL). The organic layer was washed with saturated citric acid solution then dried over anhydrous Na₂SO₄, concentrated, and purified on silica [40% EtOAc/hexane] to afford 500 mg of 3.

3 (400 mg), DCM (15 mL) mixed at 0° C., Et₃N (5.0 eq) and MsCl (2.0 eq) added at 0° C. After 15 minutes, reaction mass was diluted with DCM and water, separated the organic layer, dried over anhydrous Na₂SO₄ then concentrated to afford 400 mg of 4.

4 (400 mg), DMF (4 mL), NaN₃ (5.0 eq) mixed at 70° C. for 1 hour. Reaction mass was diluted with EtOAc and water, separated the organic layer, dried over anhydrous Na₂SO₄, filtered, concentrated, and purified on silica [25% EtOAc/hexane] to afford 100 mg of 5.

5 (300 mg), MeOH (3 mL), 10% Pd/C (50% wet, 50 mg), under H₂ at balloon pressure mixed at room temperature for 2 hours. Reaction mass filtered through pad of CELITE. The filtrate concentrated to afford 280 mg of 6.

6 (280 mg), 1,4-dioxane (15 mL), FNAB (1.0 eq), Et₃N (3.0 eq) were added at room temperature then increased to 100° C. After 16 hours the volatiles removed and reaction diluted with EtOAc and water, separated the organic layer, dried over anhydrous Na₂SO₄ and concentrated, and purified on silica [20% EtOAc/hexane] to afford 150 mg of 8 (not shown in figure).

8 (150 mg), MeOH (2 mL) mixed at 0° C., aq·NH₃ (0.2 mL) addition at 0° C. then at room temperature for 16 hours. Reaction volatiles were removed. Preparative HPLC purification isolated the product and after lyophilization 30 mg of 1106 was obtained.

### Example 39: Synthesis of Compounds 1108 ((2R,3R,4R,5S)-2-(hydroxymethyl)-1-(2-{4-[2-({2-nitro-4-[(1,2-oxazolidin-2-yl)methyl]phenyl}amino)ethyl]phenyl}ethyl)piperidine-3,4,5-triol) and 1078 ((2R,3R,4R,5S)-2-(hydroxymethyl)-1-{2-[4-(2-{[4-(1H-imidazol-2-yl)-2-nitrophenyl]amino}ethyl)phenyl]ethyl}piperidine-3,4,5-triol)

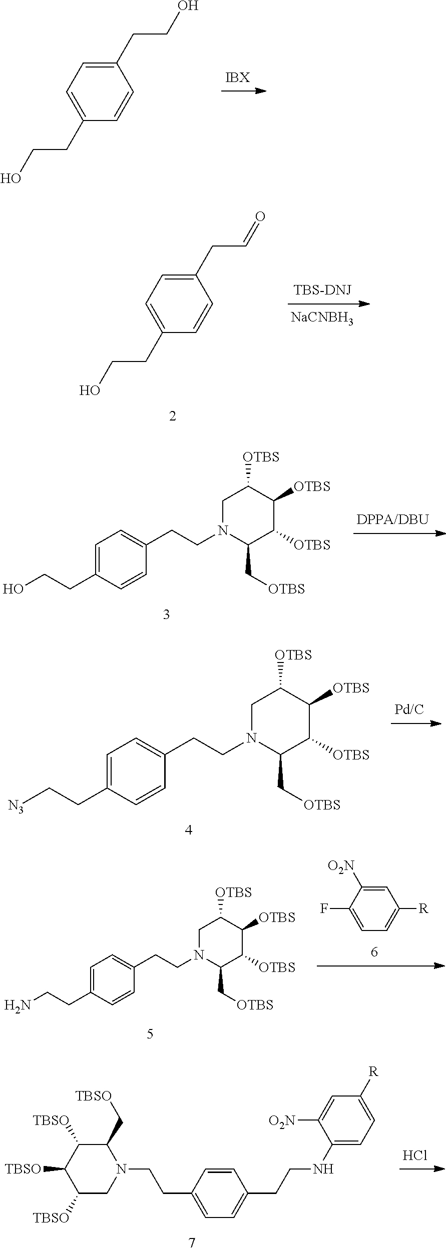

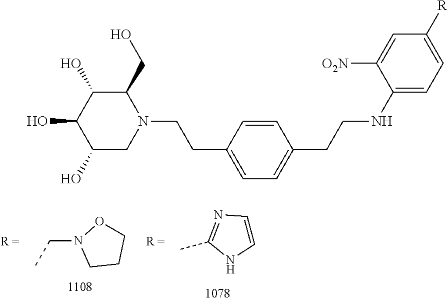

1,4-Bis(2-hydroxyethyl)benzene (4.0 g), ACN (40 mL) mixed at 0° C., IBX (1.2 eq), AcOH (1.2 eq) added, increased to room temperature. After 16 hours mix was filtered through CELITE pad, washed with EtOAc. The organic layer was washed with water, brine solution, dried over anhydrous Na₂SO₄, filtered, concentrated to afford 4.5 g of 2.

Mixed 2 (4.5 g) and MeOH at 0° C., TBS-DNJ (0.8 eq), AcOH (cat.), NaCNBH₃ (1.5 eq) added and reacted at room temperature for 16 hours. Solvent was removed. Residue was diluted with H₂O and extracted with EtOAc (2×70 mL). The combined organic layers were washed with brine solution, dried over anhydrous Na₂SO₄, filtered, concentrated. Product was purified on silica [7% EtOAc:hexane] to afford 3 (5.5 g) as pale yellow thick syrup.

3 (5.5 g) in toluene (55 mL) at 0° C., added diphenylphosphoryl azide (DPPA, 2.0 eq), 1,8-diazabicyclo[5.4.0]undec-7-ene (DBU, 2.0 eq) at 0° C. then reacted at 120° C. for 10 hours. Solvent was removed, the residue was diluted with water (60 mL) and extracted with EtOAc (2×60 mL). The combined organic layers were washed with brine, dried over anhydrous Na₂SO₄, filtered, concentrated. Product was purified on silica [3% EtOAc:hexane] to afford 4 (3.5 g) as pale yellow thick syrup.

4 (3.5 g), EtOAc (60 mL), 10% Pd/C (50% wet, 1.75 g), under mild H₂ pressure (balloon) reacted for 2 hours. Reaction mass was filtered through CELITE pad, washed with EtOAc. The combined organic layers were concentrated to afford 2.8 g of 5.

Preparation of 1108 (2R,3R,4R,5S)-2-(hydroxymethyl)-1-(2-{4-[2-({2-nitro-4-[(1,2-oxazolidin-2-yl)methyl]phenyl}amino)ethyl]phenyl}ethyl)piperidine-3,4,5-triol: Mixed 5 (800 mg), ACN (20 mL), K₂CO₃ (3.0 eq), 6 with isoxazolidine ring (Example 16, 0.7 eq) at 0° C. then increased to 90° C. After 16 hours solvent was removed, residue was diluted with water, extracted with EtOAc (2×80 mL), organic layers were washed with brine solution, dried over anhydrous Na₂SO₄ and concentrated, and residue purified by chromatography [7% EtOAc-hexane] to afford 450 mg of 7.

7 (450 mg), MeOH (9 mL) mixed at 0° C., added 4.0 MHCl in dioxane (0.45 mL) at 0° C. then increased to room temperature for 4 hours. The reaction volatiles were removed. Purification by preparative HPLC afforded 75 mg of 1108.

Preparation of 1078 (2R,3R,4R,5S)-2-(hydroxymethyl)-1-{2-[4-(2-{[4-(1H-imidazol-2-yl)-2-nitrophenyl]amino}ethyl)phenyl]ethyl}piperidine-3,4,5-triol: To prepare the substituted ring 6 for 1078, firstly 2-bromo-imidazole (2.0 g), DCM (15 mL), Et₃N (3.0 eq), (Boc)₂O (1.2 eq) were heated at room temperature for 16 hours. Reaction mass was diluted with DCM and water. The separated organic layer was dried over anhydrous Na₂SO₄ and concentrated, and purified on silica [10% EtOAc in hexane] to afford 3.0 g of N-Boc protected imidazole. Then the Boc-imidazole (500 mg), DME:H₂O (2:1, 10 mL), 4-fluoro-3-nitrophenylboronate ester (1.0 eq), Cs₂CO₃ (3.0 eq) were mixed and degassed with N₂ for 10 minutes, then Pd(dppf)Cl₂ (0.1 eq) added and heated to 80° C. for 5 hours in a sealed tube. Reaction mass was diluted with EtOAc and water. Separated the organic layer which was dried over anhydrous Na₂SO₄, concentrated, and purified by chromatography [10% then 40% EtOAc in hexane] to afford 100 mg of 6 with R=Boc-imidazole (220 mg de-Boc 6 was also recovered).

6 (150 mg), 5 (1.3 eq), Et₃N (3.0 eq), 1,4-dioxane (15 mL), were mixed at room temperature then increased to 100° C. After 16 hours reaction volatiles were removed and the mass diluted with EtOAc and water. The organic layer was dried over anhydrous Na₂SO₄ then concentrated and purified by chromatography [20% EtOAc in hexane] to afford 250 mg of 7.

7 (250 mg), MeOH (6 mL) mixed at 0° C., added 4.0 MHCl in 1,4-dioxane (2.5 mL) increased to room temperature for 2 hours. Volatiles were removed and after purification by preparative HPLC 27 mg of 1078 was obtained.

### Example 40: Synthesis of Compounds 1109 ((2R,3R,4R,5S)-2-(hydroxymethyl)-1-{2-[4-(2-{[2-nitro-4-(pyridazin-3-yl)phenyl]amino}ethyl)phenyl]ethyl}piperidine-3,4,5-triol), 1110 ((2R,3R,4R,5S)-2-(hydroxymethyl)-1-{2-[4-(2-{[2-nitro-4-(pyridin-4-yl)phenyl]amino}ethyl)phenyl]ethyl}piperidine-3,4,5-triol), 1111 ((2R,3R,4R,5S)-2-(hydroxymethyl)-1-{2-[4-(2-{[2-nitro-4-(pyrimidin-2-yl)phenyl]amino}ethyl)phenyl]ethyl}piperidine-3,4,5-triol), and 1112 ((2R,3R,4R,5S)-2-(hydroxymethyl)-1-{2-[4-(2-{[2-nitro-4-(1,3-oxazol-2-yl)phenyl]amino}ethyl)phenyl]ethyl}piperidine-3,4,5-triol)

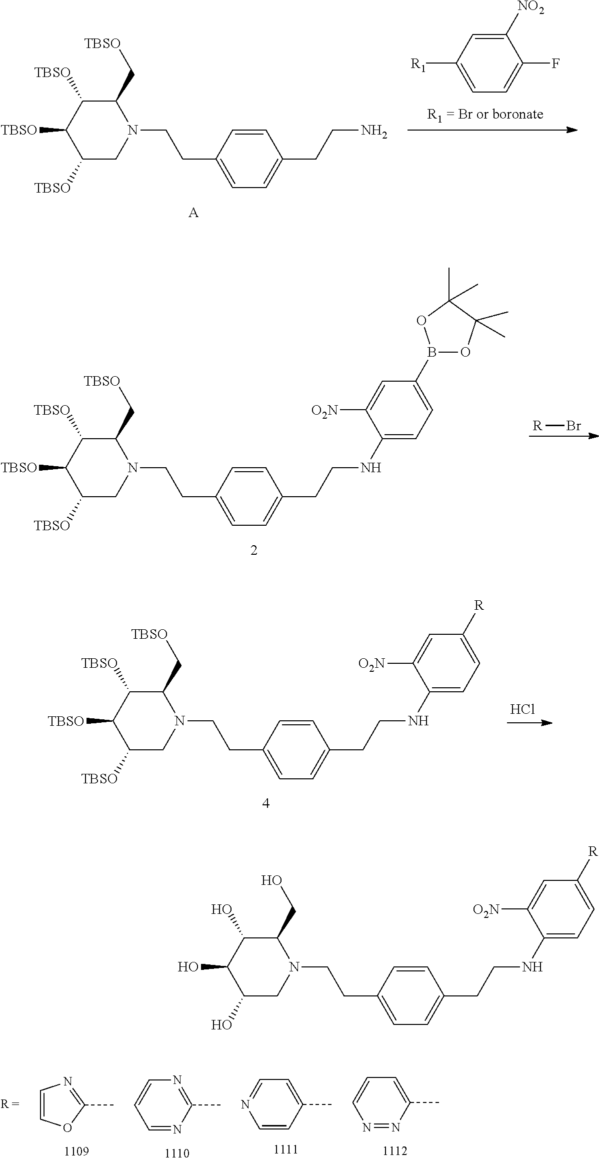

Int. 2 was prepared by two routes. In one example, 500 mg of A (Example 39), 1,4-dioxane (10 mL), Et₃N (3.0 eq), 2-(4-fluoro-3-nitrophenyl)-boronate (1.0 eq) was reacted at 100° C. for 3 hours. Solvent was removed, the residue was diluted with water and extracted with EtOAc (2×50 mL). Organic layers were washed with brine, dried over anhydrous Na₂SO₄ then concentrated to yield 600 mg of 2. In another example, A (1.0 g), 1,4-dioxane (20 mL), Et₃N (3.0 eq), 2-fluoro-5-bromo-nitrobenzene (0.8 eq) were heated to 100° C. for 3 hours. Solvent was removed and the residue was diluted with water and extracted with EtOAc (2×40 mL). Organic layers were washed with brine solution, dried over anhydrous Na₂SO₄, concentrated. and purified on silica [3% EtOAc:hexane] to afford the aryl bromide (700 mg, not shown in figure) as orange thick syrup. Then the aryl bromide (200 mg), 1,4-dioxane (10 mL), KOAc (3.0 eq) and bis-(pinacolato) diboron (1.5 eq) were degassed 20 minutes with N₂, added Pd(dppf)Cl₂ (0.1 eq), and increased to 100° C. After 16 hours solvent was removed, residue was diluted with water (20 mL) and extracted with EtOAc (2×30 mL). Organic layers were washed with brine, dried over anhydrous Na₂SO₄, filtered and concentrated to afford 2.

Preparation of 1109 (2R,3R,4R,5S)-2-(hydroxymethyl)-1-{2-[4-(2-{[2-nitro-4-(pyridazin-3-yl)phenyl]amino}ethyl)phenyl]ethyl}piperidine-3,4,5-triol: Degassed mix of 2 (300 mg), toluene:EtOH:water (1:1:1, 15 mL), 3-bromo-pyridazine (1.5 eq), Na₂CO₃ (3.0 eq) with N₂ for 20 minutes, added Pd(dppf)Cl₂ (0.1 eq) at room temperature then increased to 90° C. After 16 hours removed solvent, diluted residue with water (20 mL) and extracted with EtOAc (2×30 mL). The combined organic layers were washed with brine, dried over anhydrous Na₂SO₄, filtered, concentrated, and product was purified on silica [25% EtOAc:hexane] to afford 4 (110 mg) as yellow thick syrup.

4 (110 mg), MeOH (2.2 mL), 4.0M HCl in 1,4-dioxane (1.1 mL) added at 0° C. then increased to room temperature. After 6 hours volatiles were removed, and purified by preparative HPLC to afford 25 mg of 1109.

Preparation of 1110 (2R,3R,4R,5S)-2-(hydroxymethyl)-1-{2-[4-(2-{[2-nitro-4-(pyridin-4-yl)phenyl]amino}ethyl)phenyl]ethyl}piperidine-3,4,5-triol: 1 (200 mg), pyridine-4-boronate (1.5 eq), toluene:EtOH:water (1:1:1, 15 mL), Na₂CO₃ (3.0 eq) degassed with N₂ 20 minutes, added Pd(dppf)Cl₂ (0.1 eq) and increased temperature to 90° C. After 16 hours solvent was removed, the residue diluted with water (20 mL) and extracted with EtOAc (2×20 mL). Combined organic layers were washed with brine, dried over anhydrous Na₂SO₄, filtered, concentrated, and purified by column [15% EtOAc:hexane] to afford 4 (110 mg) as thick yellow syrup.

4 was deprotected by mixing 110 mg with MeOH (2.2 mL) at 0° C., added 4.0 M HCl in 1,4-dioxane (1.1 mL), increased to room temperature for 6 hours. Solvent was removed. After preparative HPLC purification 10 mg of 1110 was obtained.

Preparation of 1111 (2R,3R,4R,5S)-2-(hydroxymethyl)-1-{2-[4-(2-{[2-nitro-4-(pyrimidin-2-yl)phenyl]amino}ethyl)phenyl]ethyl}piperidine-3,4,5-triol: 2 (200 mg), 2-bromopyrimidine (1.5 eq), toluene:EtOH:water (1:1:1, 15 mL), Na₂CO₃ (3.0 eq), degassed with N₂ for 20 minutes, Pd(dppf)Cl₂ (0.1 eq) addition at room temperature, then 90° C. for 16 hours. Solvent was removed, the residue was diluted with water (20 mL) and extracted with EtOAc (2×20 mL). The combined organic layers were washed with brine, dried over anhydrous Na₂SO₄, filtered, concentrated, and purified on silica [using 15% EtOAc:hexane eluent] to afford 4 (110 mg) as yellow thick syrup.

4 (110 mg), MeOH (2.2 mL), 4.0M HCl in 1,4-dioxane (1.1 mL) mixed at 0° C. then room temperature for 6 hours. Solvent was removed and preparative HPLC purification gave 10 mg 1111.

Preparation of 1112 (2R,3R,4R,5S)-2-(hydroxymethyl)-1-{2-[4-(2-{[2-nitro-4-(1,3-oxazol-2-yl)phenyl]amino}ethyl)phenyl]ethyl}piperidine-3,4,5-triol: Degassed 2 (600 mg), toluene:EtOH:water (1:1:1, 15 mL), 2-bromooxazole (1.0 eq), Na₂CO₃ (3.0 eq) with N₂ for 20 minutes. Added Pd(dppf)Cl₂ (0.1 eq) at room temperature and increased to 90° C. After 16 hours solvent was removed, the residue was diluted with water (20 mL) and extracted with EtOAc (2×50 mL). The combined organic layers were washed with brine, dried over anhydrous Na₂SO₄, filtered, concentrated, and purified on silica [7% EtOAc:hexane] to afford 4 (120 mg) as orange thick syrup.

4 (120 mg), MeOH (2.2 mL), 4.0 M HCl in 1,4-dioxane (1.2 mL) mixed at 0° C. then increased to room temperature for 2 hours. Combined batches were purified by preparative HPLC to afford 45 mg of 1112.

### Example 41: Synthesis of Compound 1125 (N-[3-(pyrimidin-2-yl)-5-({6-[(2R,3R,4R,5S)-3,4,5-trihydroxy-2-(hydroxymethyl)piperidin-1-yl]hexyl}amino)phenyl]acetamide)

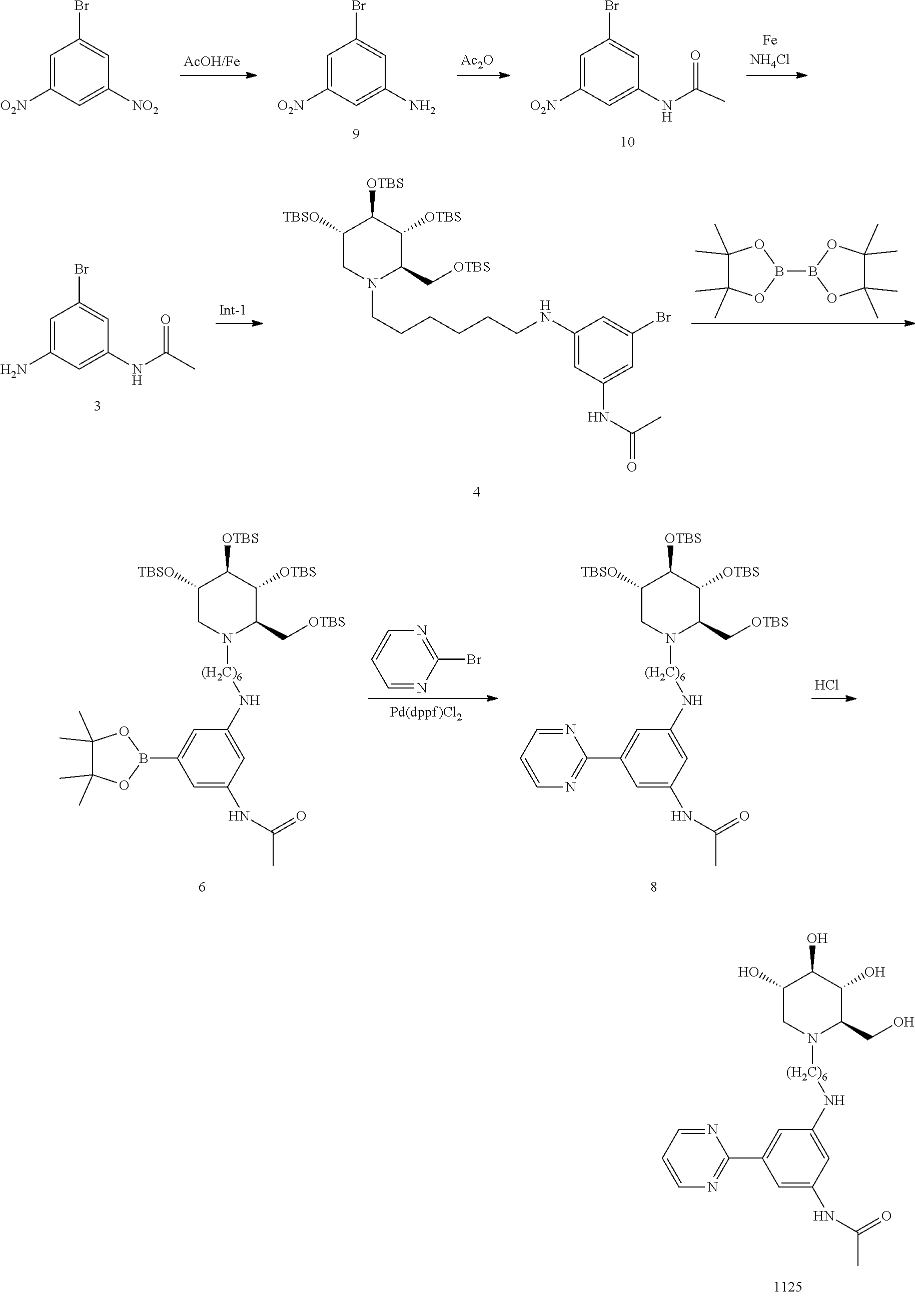

3-Bromo-dinitrobenzene (6 g), AcOH (36 mL) heated to 90° C. Iron powder (Fe, 2.5 eq) was added, temperature maintained for 30 minutes, then the reaction was cooled to room temperature and quenched with crushed ice. The precipitate was filtered and washed with water and dried under air. The solids were dissolved in DCM and filtered through CELITE bed. The filtrate was concentrated to afford 4 g of 9 as orange solid.

9 (4 g) at 0° C. mixed with Ac₂O (130 mL) and increased to room temperature for 30 minutes. The reaction mixture was quenched with crushed ice. The precipitate was filtered and washed with water and dried to afford 4 g of 10 as off-white solid.

10 (4 g), Fe (2.5 eq), saturated aq. NH₄Cl (4 mL), EtOH (150 mL) maintained at 80° C. for 4 hours. The reaction was cooled to room temperature and filtered through CELITE bed and washed with EtOAc. The filtrate was concentrated and purified on silica [50% EtOAc/hexane] to afford 3 g of 3 as a solid.

Mixed 3 (1.2 eq), Int-1 (Example 1, 1 g), MeOH (5 mL), acetic acid (cat.) at 0° C. then NaCNBH₃ (1.5 eq) added at 0° C. and increased to room temperature. After 3 hours the reaction was concentrated. The residue was dissolved in water and extracted with EtOAc (2×10 mL). The organic layer was dried over Na₂SO₄, filtered, concentrated, and purified on silica [50% EtOAc-hexane] to afford 400 mg of 4 as colorless syrup.

4 (350 mg), bis(pinacolato) diboron (2.2 eq), KOAc (3.0 eq), Pd(dppf)Cl₂ (0.1 eq), 1,4-dioxane (10 mL), degassed 15 minutes N₂, reacted at 90° C. for 3 hours. Reaction was concentrated then residue was diluted with water and extracted with EtOAc (2×20 mL). The organic layer was dried over anhydrous Na₂SO₄ and concentrated to afford 350 mg of 6.

6 (350 mg), toluene:EtOH:water (25 mL), 2-bromopyrimidine (1 eq), Pd(dppf)Cl₂ (0.1 eq), Na₂CO₃ (3 eq) degassed 15 minutes N₂, reacted at 70° C. After 16 hours the reaction mass was cooled to room temperature and diluted with EtOAc and water. Separated the organic layer and dried over anhydrous Na₂SO₄ and concentrated. Combined batches were purified on silica [50:50 EtOAc:hexane] to afford 200 mg of 8.

8 (140 mg), MeOH/DCM (4 mL), 4.0 M HCl in dioxane (1.4 mL) mixed at room temperature for 4 hours. Preparative HPLC purification yielded 15 mg of 1125.

### Example 42: Synthesis of Compound 1114 (N-[3-(pyrimidin-2-yl)-5-({6-[(2R,3R,4R,5S)-3,4,5-trihydroxy-2-(hydroxymethyl)piperidin-1-yl]hexyl}amino)phenyl]cyclopropanesulfonamide)

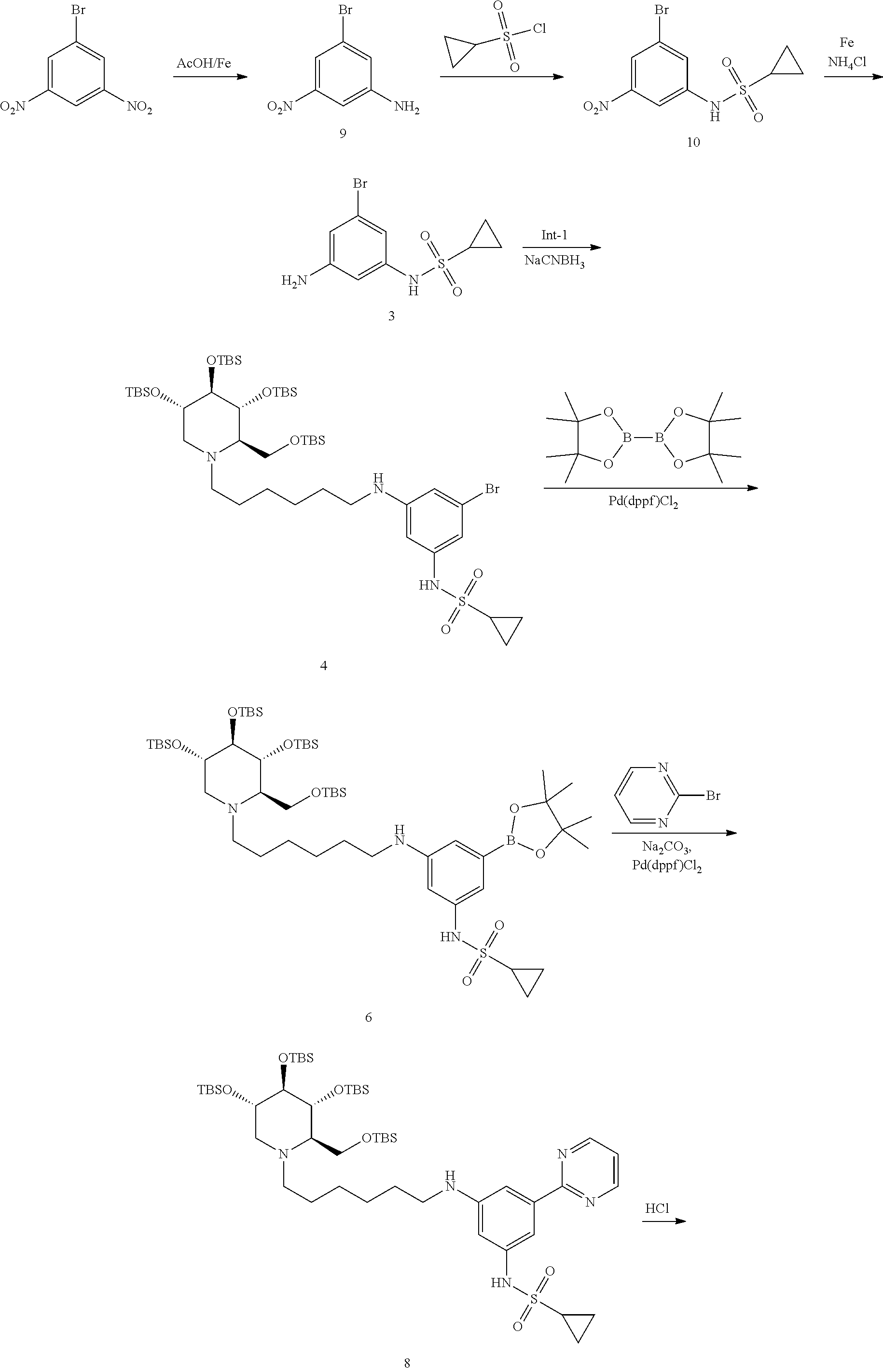

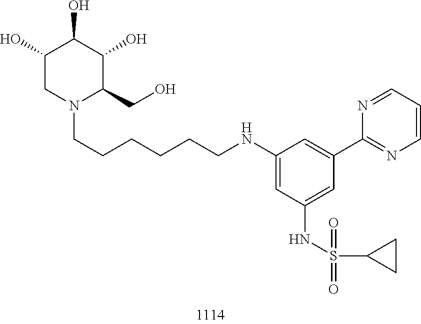 [SMILES: O=S(=O)(Nc1cc(NCCCCCCN2C[C@H](O)[C@@H](O)[C@H](O)[C@H]2CO)cc(-c2ncccn2)c1)C1CC1]

1-Bromo-3,5-dinitrobenzene (3 g) was suspended in AcOH (18 mL). Fe powder (2.5 eq) was added and reaction mix held at 90° C. for 30 minutes. The reaction was cooled to room temperature and quenched with crushed ice. The precipitate was filtered and washed with water and dried. The solids were dissolved in DCM and filtered through CELITE bed. The filtrate was dried over Na₂SO₄, filtered, concentrated to afford 2.0 g of 9.

9 (1.7 g), DCM (70 mL), pyridine (3 eq), cyclopropane sulphonyl chloride (1.8 eq), 4-dimethylaminopyridine (cat) mixed at room temperature for 36 hours. The reaction was quenched with water and extracted with DCM (2×50 mL). The organic was dried over anhydrous Na₂SO₄, filtered, concentrated, and purified on silica [20% EtOAc-hexane] to afford 1.9 g of 10.

10 (1.9 g), EtOH (80 mL), Fe (6 eq), saturated aq. NH₄Cl (6.3 mL) heated at 90° C. for 4 hours. The reaction was cooled to room temperature and diluted with EtOAc. The reaction mass was filtered through CELITE bed and washed with excess EtOAc (100 mL). The filtrate was concentrated, the residue dissolved in EtOAc and washed with water, dried over Na₂SO₄, filtered, concentrated, and purified on silica [20% EtOAc in hexane] to afford 1.5 g of 3.

Mixed 3 (1 eq), Int-1 (3.0 g), MeOH (70 mL), AcOH (0.1 mL), NaCNBH₃ (1.5 eq) at room temperature for 16 hours. Solvent was removed. The residue was dissolved in EtOAc and washed with water, organic layer was dried over Na₂SO₄, filtered, concentrated, and purified on silica [10% EtOAc/hexane] to afford 1.9 g of 4 as colorless syrup.

To 4 (500 mg), bis(pinacolato) diboron (2.2 eq), 1,4-dioxane (20 mL) and KOAc (3 eq), added Pd(dppf)Cl₂ (0.1 eq) degassed 15 minutes N₂ at room temperature, reacted at 100° C. 3 hours. The reaction was cooled to room temperature, quenched with ice-cold water and extracted with EtOAc (2×20 mL). The organic layer was dried over Na₂SO₄, filtered, concentrated to afford 500 mg of 6.

To 6 (500 mg), 2-bromopyrimidine (1 eq), toluene:EtOH:water (1:1:1. 20 mL), Na₂CO₃ (3 eq) degassed 15 minutes N₂, added Pd(dppf)Cl₂ (0.1 eq), maintained at 80° C. for 16 hours. The reaction was cooled to room temperature, diluted with water and extracted with EtOAc (20 mL). The organic layer was dried over anhydrous Na₂SO₄, filtered, concentrated, and purified on silica [15% EtOAc in hexane] to afford 200 mg of 8.

8 (200 mg), MeOH (2 mL), 4.0 MHCl in dioxane (1 mL) mixed at room temperature. After 3 hours the volatiles were removed. Purification by preparative HPLC gave 50 mg of 1114.

### Example 43: Synthesis of Compound 1075 ((2R,3R,4R,5S)-1-[2-(1-{2-[(3-bromo-5-methoxyphenyl)amino]ethyl}-1H-indol-3-yl)ethyl]-2-(hydroxymethyl)piperidine-3,4,5-triol)

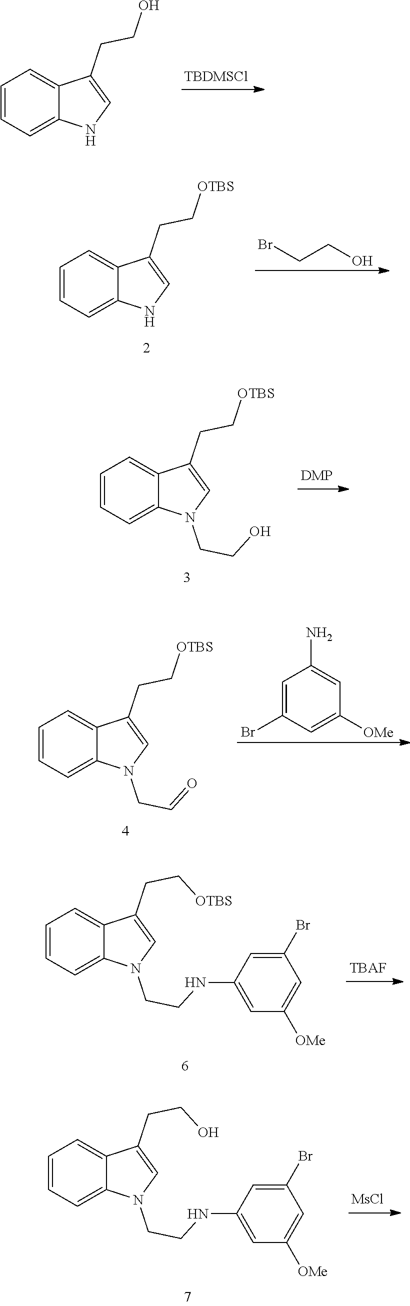

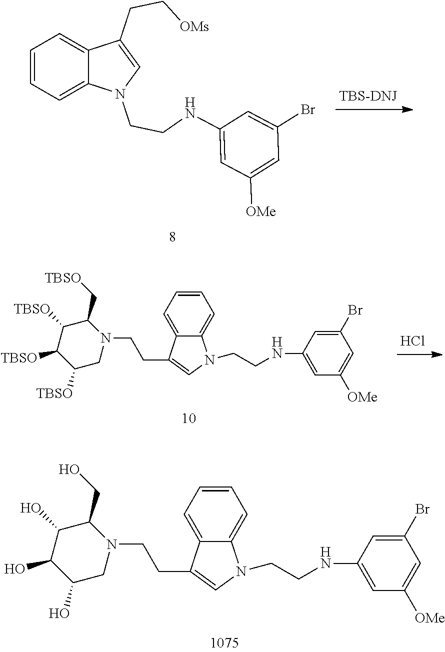

3-(2-Hydroxyethyl)indole (16 g), DMF (120 mL) mixed at 0° C., imidazole (2.0 eq), TBDMSCl (1.1 eq) added and temperature increased to room temperature. The reaction was diluted with water (2×500 mL) and extracted with EtOAc (2×500 mL). The organic layer was dried over anhydrous Na₂SO₄ and concentrated. Material was purified by chromatography [3% EtOAc:hexane] to afford 2 (26 g) as colorless thick syrup.

2 (25.0 g), DMF (125 mL) mixed at 0° C., NaH (2.0 eq) mixed for 30 minutes at same temperature, 2-bromoethanol (2.0 eq) added at 0° C. then reacted at room temperature for 16 hours. The reaction was poured into ice cold water and extracted with EtOAc. The organic layer was dried over anhydrous Na₂SO₄, filtered, concentrated. Material was purified on silica [10-15% of EtOAc in hexane] to obtain 12 g of 3 as colourless syrup.

3 (2.0 g), DCM (50 mL) mixed at 0° C., DMP (1.5 eq) added and increased to room temperature for 1.5 hours. Reaction mass was filtered through CELITE bed, washed with DCM. The organic layer was washed with saturated NaHCO₃ solution, water and dried over anhydrous Na₂SO₄, and concentrated to afford 2.2 g of 4.

4 (2.2 g), 3-bromo-5-methoxyaniline (0.8 eq), MeOH (50 mL) mixed at 0° C., AcOH (cat) then after 30 minutes NaCNBH₃ (1.5 eq) addition at 0° C. then increased to room temperature. After 16 hours volatiles were removed. Product was taken into DCM and washed with water. Organic layer was dried over anhydrous Na₂SO₄, filtered, concentrated, and purified by column [7-10% of EtOAc in hexane] to obtain 500 mg of 6 as colourless syrup.

6 (500 mg), THE (20 mL), mixed at 0° C., TBAF (1.0 M in THF, 1.0 eq) at same temperature for 30 minutes then room temperature for 2 hours. The volatiles were removed. Mass was taken into EtOAc and washed with water. The organic layer was washed with brine, dried over anhydrous Na₂SO₄, filtered, concentrated. Material was purified by column [20-30% EtOAc in hexane] to obtain 250 mg of 7 as colourless syrup.

To 7 (250 mg) in DCM (10 mL) added DIPEA (3.0 eq) and MsCl (1.0 eq) at 0° C., mixed at room temperature for 1 hour. Reaction was diluted with DCM, washed with saturated NaHCO₃ solution, dried over anhydrous Na₂SO₄, filtered, and concentrated to obtain 300 mg of 8.

8 (300 mg), ACN (5 mL), TBS-DNJ (1.0 eq), K₂CO₃ (3.0 eq), added at room temperature, then increased to 70° C. for 2 hours. The reaction was diluted with EtOAc and washed with water. The organic layer was dried over anhydrous Na₂SO₄, concentrated, and purified by column [20% of EtOAc in hexane] to yield 100 mg of thick syrup 10.

10 (100 mg), DCM (5.0 mL), 4.0M HCl in 1,4-dioxane (1.0 mL) added at 0° C. then to room temperature. After 16 hours volatiles were removed. After purification by preparative HPLC 10.0 mg of 1075 was obtained as off-white solid.

### Example 44: Synthesis of Compound 1076 ((2R,3R,4R,5S)-2-(hydroxymethyl)-1-(2-{1-[2-(phenylamino)ethyl]-1H-indol-3-yl}ethyl)piperidine-3,4,5-triol)

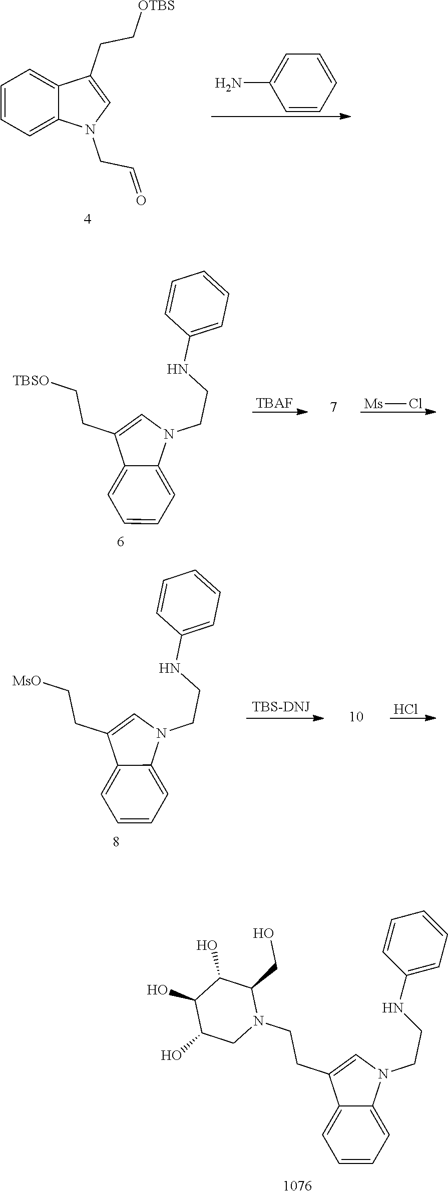

4 (from Example 43, 1.0 g), aniline (1.0 eq), MeOH (20 mL) were mixed at 0° C., then AcOH (cat) for 20 minutes then NaCNBH₃ (1.5 eq) addition at 0° C. then room temperature. After 16 hours volatiles were removed. Residue was solubilized in DCM and washed with water. Organic layer was dried over anhydrous Na₂SO₄, filtered, concentrated, and purified on silica [⁸-10% of EtOAc-hexane] to obtain 600 mg of 6 as colourless syrup.

6 (600 mg), THE (10 mL) mixed at 0° C., TBAF (1.0 M in THF, 1.0 eq) added at 0° C. for 30 minutes then increased to room temperature for 2 hours. Solvent was removed. Residue was diluted with water and extracted with EtOAc (2×10 mL). The organic layer was washed with brine solution, dried over anhydrous Na₂SO₄, concentrated. Material was purified by column chromatography [45-50% of EtOAc in hexane] to obtain 300 mg of primary alcohol 7 (not shown on figure) as colorless syrup.

Mixed 7 (300 mg) in DCM (10 mL) and added DIPEA (3.0 eq) and MsCl (1.1 eq) at 0° C., increased to room temperature for 1 hour. Diluted reaction with cold water, extracted with DCM. The organic layer was dried over anhydrous Na₂SO₄ and concentrated to afford 350 mg of 8.

8 (350 mg), ACN (5 mL), TBS-DNJ (1.1 eq), K₂CO₃ (3.0 eq) added at room temperature then refluxed for 16 hours. The reaction was cooled to room temperature, diluted with EtOAc. The organic layer was dried over anhydrous Na₂SO₄ and concentrated. Product was purified on silica [60-120 mesh, eluted with 5-10% of EtOAc in hexane] to obtain 150 mg of TBS-protected product (10, not shown on figure) as colorless syrup.

10 (150 mg) was deprotected by suspending in MeOH (3.0 mL) with 4.0 M HCl in 1,4-dioxane (1.5 mL) added at 0° C. then reacted at room temperature for 4 hours. Volatiles were removed and after purification by preparative HPLC 10 mg of 1076 was obtained.

### Example 45: Synthesis of Compound 1077 ((2R,3R,4R,5S)-1-[2-(3-{2-[(4-azido-2-nitrophenyl)amino]ethyl}-5-(propan-2-yl)phenyl)ethyl]-2-(hydroxymethyl)piperidine-3,4,5-triol)

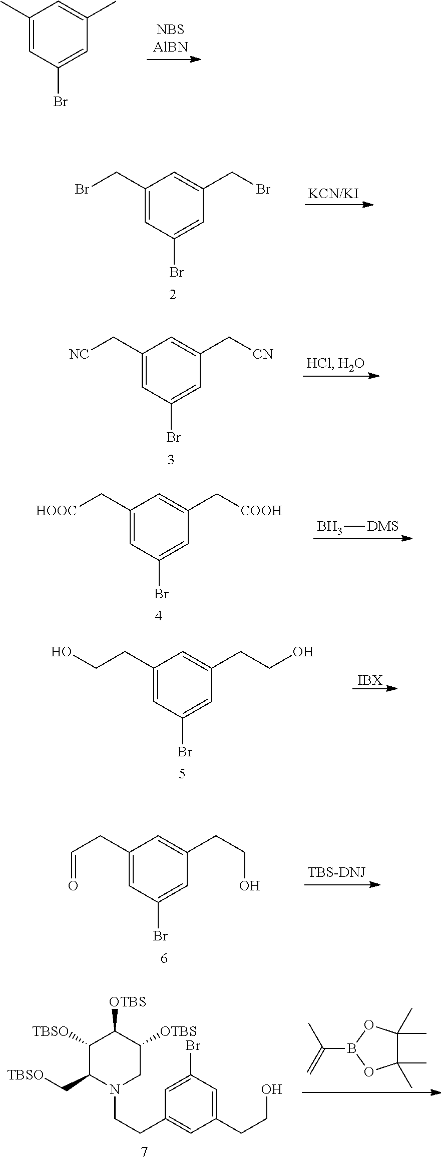

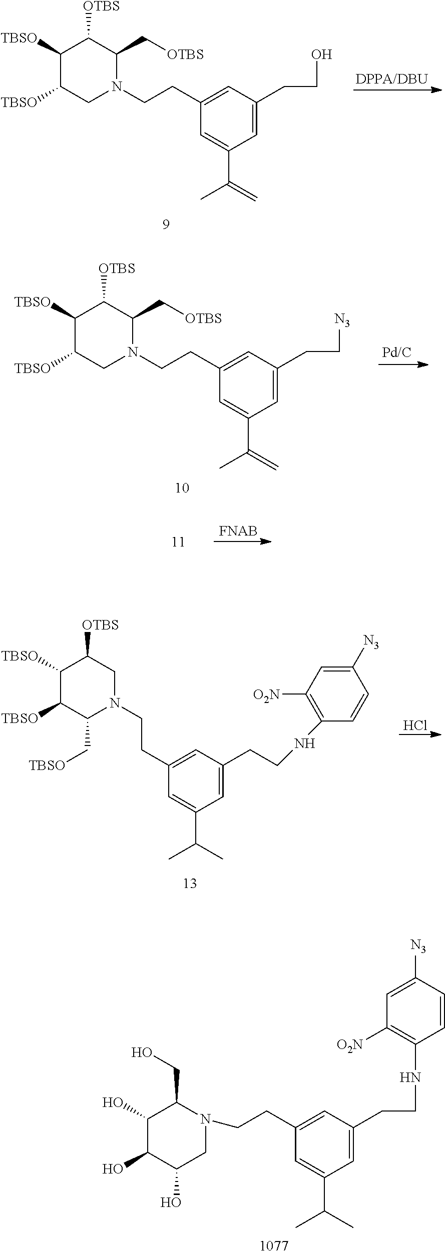

1-Bromo-3,5-dimethylbenzene (75 g), ACN (800 mL), N-bromosuccinimide (NBS, 2.1 eq), azobisisobutyronitrile (AIBN, 500 mg) mixed at 80° C. for 8 hours. Solvent was removed and the residue was diluted with water and extracted with EtOAc. The organic layer was dried over anhydrous Na₂SO₄ and concentrated. Product was crystalized with methanol to afford 60 g of 2 as white solid.

2 (30 g), ACN (520 mL) mixed at 0° C., KCN (2.2 eq), KI (0.1 eq), tetrabutylammonium iodide (TBAI, 0.2 eq), H₂O (75 mL) added and reacted at room temperature for 20 hours. Solvent was removed. Residue was diluted with water and extracted with EtOAc. The organic layer was washed with brine solution, dried over anhydrous Na₂SO₄ and concentrated. Material was purified on silica [30% EtOAc-hexane] to afford the 16 g of 3 as white solid.

Refluxed 3 (16.0 g) and concentrated HCl (160 mL) for 12 hours then cooled to room temperature and diluted with water. The solid was filtered and dried to afford 15.0 g of 4 as off white solid.

Mixed 4 (15 g) and THE at 0° C., H₃B-DMS (4.0 eq) added and warmed to room temperature. After 3 hours the reaction was quenched with 1.0 N HCl solution and diluted with water then extracted with EtOAc. The organic layer was washed with brine solution, dried over anhydrous Na₂SO₄, concentrated. Material was purified on silica [3% MeOH in DCM] to afford 9.0 g of 5 as colourless thick syrup.

Mixed 5 (9.0 g), ACN (90 mL) at 0° C., added IBX (1.2 eq), AcOH (1.2 eq) then mixed at room temperature for 12 hours. Reaction mix was filtered through CELITE pad, washed with EtOAc, and concentrated to afford 8.0 g of 6.

6 (8.0 g), MeOH mixed at 0° C., TBS-DNJ (0.8 eq), AcOH (cat.), NaCNBH₃ (1.5 eq) added then increased to room temperature for 16 hours. Solvent was removed and the residue was diluted with water and extracted with EtOAc. The organic layer was washed with brine solution, dried over anhydrous Na₂SO₄ and concentrated. Material was purified on silica [5% EtOAc-hexane] to afford 4.5 g of 7 as colorless liquid.

Degassed 7 (1.2 g), prop-1-en-2-yl boronate (1.2 eq), 1,4-dioxane:water (3:1, 12 mL), Na₂CO₃ (3.0 eq) with N₂ 10 minutes, added Pd(PPh₃)₄ (0.1 eq) then heated 16 hours at 80° C. Volatiles were removed. The residue was diluted with water and extracted with EtOAc (2×100 mL). The organic layer was dried over anhydrous Na₂SO₄ then concentrated, and purified on silica [3% EtOAc:hexane] to afford 620 mg of 9 as colorless thick syrup.

9 (600 mg), DPPA (1.5 eq), DBU (1.5 eq), toluene (15 mL) added at 0° C., then increased to 80° C. for 12 hours. Reaction volatiles were removed, and purified on silica [2% EtOAc:hexane] to afford 350 mg of 10 as colorless thick syrup.

10 (350 mg), EtOAc (5 mL), 10% Pd/C (150 mg, 50% wet) held under moderate H₂ pressure (balloon) at room temperature for 4 hours. Reaction was filtered through CELITE pad, washed with EtOAc and filtrate was concentrated to afford 250 mg of amine 11 (not shown on figure).

Mixed amine 11 (250 mg), FNAB (0.8 eq), Et₃N (3.0 eq), 1,4-dioxane (5 mL), maintained at 90° C. After 16 hours volatiles were removed, and purified on silica [3% EtOAc:hexane] to afford 150 mg of 13 as yellow syrup.

13 (150 mg), 4.0M HCl in 1,4-dioxane (1.5 mL), MeOH (5 mL) were added at 0° C. then increased to room temperature for 12 hours. Reaction volatiles were removed to obtain 110 mg of material which yielded 12 mg after preparative HPLC purification. Material was combined with another batch (input 80 mg of 13) and purified on silica [7% MeOH:DCM] to afford 18.0 mg of 1077 as yellow semi-solid.

### Example 46: Synthesis of Compound 1115 ((2R,3R,4R,5S)-1-[2-(3-{2-[(4-azido-2-nitrophenyl)amino]ethyl}-5-cyclohexylphenyl)ethyl]-2-(hydroxymethyl)piperidine-3,4,5-triol)

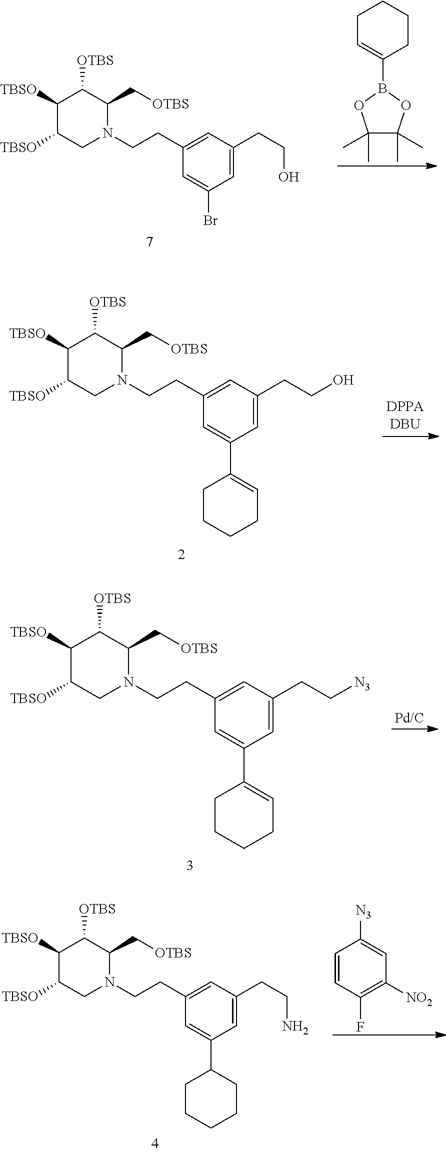

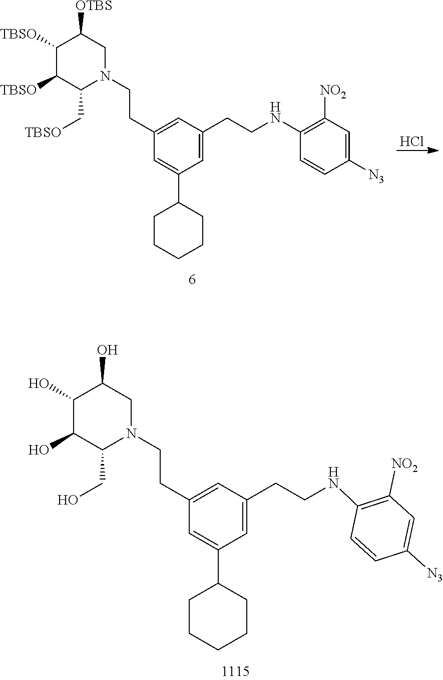 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCc1cc(CCNc2ccc(N=[N+]=[N-])cc2[N+](=O)[O-])cc(C2CCCCC2)c1 ; Cl ; [N-]=[N+]=Nc1ccc(NCCc2cc(CCN3C[C@H](O)[C@@H](O)[C@H](O)[C@H]3CO)cc(C3CCCCC3)c2)c([N+](=O)[O-])c1]

7 prepared as Example 45 (500 mg), 1-cyclohexen-1-yl-boronate (1.2 eq), 1,4-dioxane:water (3:1, 12 mL), Na₂CO₃ (3.0 eq) degassed with N₂ 10 minutes, then Pd(PPh₃)₄ (0.1 eq) added and increased to 80° C. After 16 hours reaction volatiles were removed. The residue was diluted with water and extracted with EtOAc (2×50 mL). The organic layer was dried over anhydrous Na₂SO₄ then concentrated, and purified on silica [3% EtOAc:hexane] to afford 300 mg of 2 as colorless thick syrup.

2 (300 mg), DPPA (1.5 eq), DBU (1.5 eq), toluene (6 mL) added at 0° C., then increased to 80° C. for 12 hours. Reaction volatiles were removed, and purified on silica [3% EtOAc:hexane] to afford 200 mg of 3 as colorless thick syrup.

3 (200 mg), EtOAc (5 mL), 10% Pd/C (100 mg, 50% wet) held under H₂ balloon pressure at room temperature for 4 hours. Reaction was filtered through CELITE pad, washed with EtOAc, and filtrate was concentrated to provide 4.

4 (200 mg), FNAB (0.8 eq), Et₃N (3.0 eq), 1,4-dioxane (5 mL) held at 90° C. for 16 hours. Volatiles were removed and residue was purified on silica [3% EtOAc:hexane] to afford 100 mg of 6 as yellow syrup.

6 (100 mg), 4.0M HCl in 1,4-dioxane (1.0 mL), MeOH (4 mL) added at 0° C. then room temperature for 16 hours. Volatiles were removed and residue purified by preparative HPLC to obtain 12.0 mg of 1115 as yellow solid.

### Example 47: Synthesis of Compounds 1113 ((2R,3R,4R,5S)-1-{2-[4-(2-{[3-chloro-5-(1,2,4-oxadiazol-3-yl)phenyl]amino}ethyl)phenyl]ethyl}-2-(hydroxymethyl)piperidine-3,4,5-triol), 1120 ((2R,3R,4R,5S)-1-{2-[4-(2-{[3-(5-cyclobutyl-1,2,4-oxadiazol-3-yl)-5-methoxyphenyl]amino}ethyl)phenyl]ethyl}-2-(hydroxymethyl)piperidine-3,4,5-triol), ((2R,3R,4R,5S)-1-{2-[4-(2-{[3-(5-butyl-1,2,4-oxadiazol-3-yl)-5-methoxyphenyl]amino}ethyl)phenyl]ethyl}-2-(hydroxymethyl)piperidine-3,4,5-triol), 1122 ((2R,3R,4R,5S)-1-{2-[4-(2-{[3-chloro-5-(5-cyclobutyl-1,2,4-oxadiazol-3-yl)phenyl]amino}ethyl)phenyl]ethyl}-2-(hydroxymethyl)piperidine-3,4,5-triol), and 1123 ((2R,3R,4R,5S)-1-{2-[4-(2-{[3-chloro-5-(5-propyl-1,2,4-oxadiazol-3-yl)phenyl]amino}ethyl)phenyl]ethyl}-2-(hydroxymethyl)piperidine-3,4,5-triol)

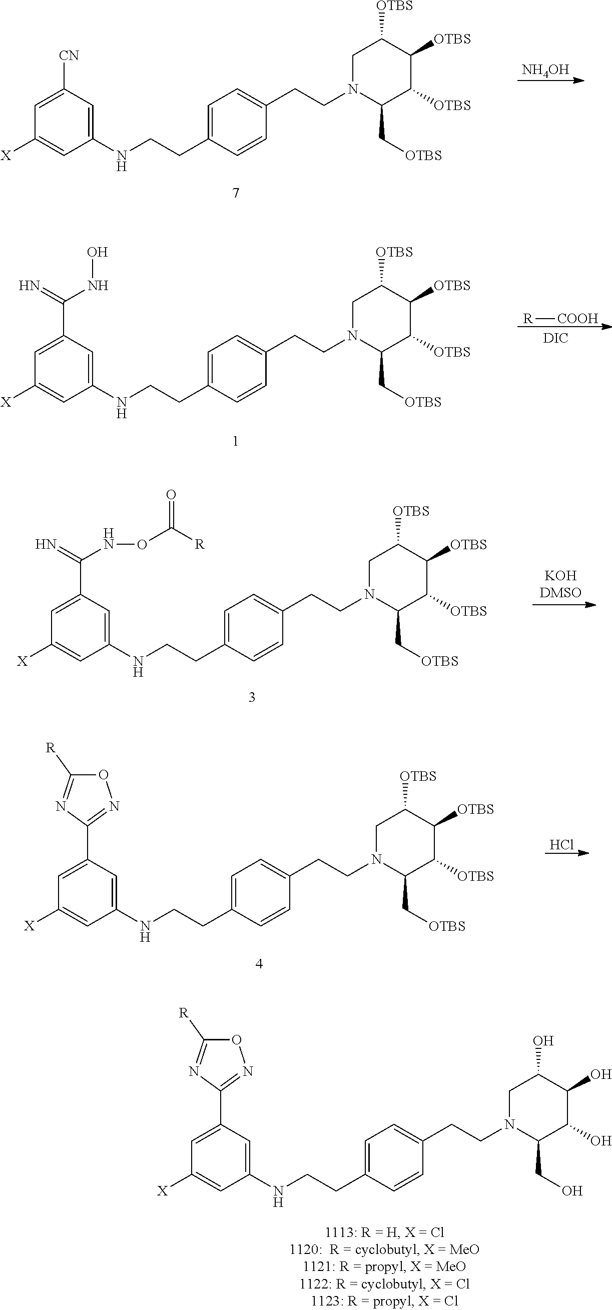

Preparation of 1120 (2R,3R,4R,5S)-1-{2-[4-(2-{[3-(5-cyclobutyl-1,2,4-oxadiazol-3-yl)-5-methoxyphenyl]amino}ethyl)phenyl]ethyl}-2-(hydroxymethyl)piperidine-3,4,5-triol: 7 (prepared as for Example 50, 2.4 g), EtOH (35 mL), aq. NH₄OH (7.2 mL) heated at 90° C. for 5 hours. Solvent was removed. The residue was diluted with water and EtOAc. The organic layer was dried over anhydrous Na₂SO₄ then concentrated, and purified on silica [50% EtOAc in hexane] to afford 820 mg of 1 as colorless thick syrup.

Cyclobutanecarboxylic acid (0.8 eq), DMF (2 mL), HOBt (1.0 eq), N,N′-diisopropyl-carbodiimide (DIC, 1.0 eq) were mixed at 0° C. for 30 minutes then 1 (250 mg) added at the same temperature then held at room temperature for 5 hours. Solvent was removed. The residue was diluted with water and EtOAc. The organic layer was dried over anhydrous Na₂SO₄ and concentrated, and purified on silica [20% EtOAc in hexane] to afford 200 mg of 3 as colorless thick syrup.

3 (200 mg), KOH (1.0 eq), DMSO (5 mL) mixed at room temperature for 1 hour. The reaction was diluted with water and extracted with EtOAc (2×5 mL). The organic layer was dried over anhydrous Na₂SO₄, concentrated, and purified on silica [10% EtOAc-hexane] to afford 160 mg of 4 as colorless thick syrup.

4 (160 mg), 4.0 M HCl in 1,4-dioxane (2 mL), 1,4-dioxane (3 mL) mixed at 0° C. then maintained at room temperature for 6 hours. The volatiles were concentrated and after purification by preparative HPLC 20 mg of 1120 was obtained.

Preparation of 1121 (2R,3R,4R,5S)-1-{2-[4-(2-{[3-(5-butyl-1,2,4-oxadiazol-3-yl)-5-methoxyphenyl]amino}ethyl)phenyl]ethyl}-2-(hydroxymethyl)piperidine-3,4,5-triol: Butanoic acid (1.0 eq), DMF (5 mL), 0° C., DIC (1.0 eq), HOBt (1.0 eq), 1 prepared as for 1120 (350 mg) mixed at 0° C. then held at room temperature for 5 hours. Solvent was removed. The residue was diluted with water and EtOAc. The organic layer was dried over anhydrous Na₂SO₄ and concentrated, and purified on silica [20% EtOAc/hexane] to afford 270 mg of 3 as colorless thick syrup.

3 (270 mg), KOH (1.0 eq), DMSO (5 mL) mixed at room temperature for 1 hour. The reaction was diluted with water and extracted with EtOAc (2×5 mL). The organic layer was dried over anhydrous Na₂SO₄ concentrated to afford 4.

4 (200 mg), 4.0 M HCl in 1,4-dioxane (2 mL), 1,4-dioxane (3 mL) added together at 0° C. then held at room temperature for 6 hours. The volatiles were concentrated. After purification by preparative HPLC 30 mg of 1121 was obtained.

Preparation of 1122 (2R,3R,4R,5S)-1-{2-[4-(2-{[3-chloro-5-(5-cyclobutyl-1,2,4-oxadiazol-3-yl)phenyl]amino}ethyl)phenyl]ethyl}-2-(hydroxymethyl)piperidine-3,4,5-triol: 7 (prepared as in Example 51, 2.5 g), EtOH (60 mL), aq. NH₂OH (6 mL) held at 90° C. for 8 hours. The reaction was cooled to room temperature then concentrated. The residue was purified on silica [30% EtOAc in hexane] to afford 2.0 g of 1.

Cyclobutanecarboxylic acid (0.8 eq), DMF (6 mL), HOBt (1.0 eq), DIC (1.0 eq) mixed at 0° C. for 30 minutes then 1 (400 mg) added at 0° C. and held at room temperature for 16 hours. The reaction was diluted with water and extracted with EtOAc (2×30 mL). The organic layer was dried over anhydrous Na₂SO₄ then concentrated, and purified on silica [20% EtOAc:hexane] to afford 230 mg of 3.

3 (230 mg), KOH (1.0 eq), DMSO (3 mL) mixed at room temperature for 1 hour. The reaction was diluted with water and extracted with EtOAc (2×25 mL). The organic layer was dried over anhydrous Na₂SO₄ concentrated to afford 180 mg of 4.

4 (180 mg), 4.0 M HCl in 1,4-dioxane (2 mL), 1,4-dioxane (5 mL) mixed at 0° C. then at room temperature for 6 hours. The reaction was concentrated. After purification by preparative HPLC 30 mg of 1122 was obtained.

Preparation of 1123 (2R,3R,4R,5S)-1-{2-[4-(2-{[3-chloro-5-(5-propyl-1,2,4-oxadiazol-3-yl)phenyl]amino}ethyl)phenyl]ethyl}-2-(hydroxymethyl)piperidine-3,4,5-triol: Butanoic acid (1.0 eq), DMF (6 mL) mixed at 0° C., HOBt (1 eq) and DIC (1 eq) added at 0° C., added 1 prepared as for 1122 (400 mg) then increased to room temperature. After 16 hours the reaction mixture was quenched with ice-cold water and extracted with EtOAc (2×20 mL). The organic layer was washed with water and dried over Na₂SO₄, filtered, concentrated, and purified on silica [15% EtOAc:hexane] to afford 250 mg of 3.

3 (250 mg), DMSO (7 mL), KOH (2 eq) held at room temperature for 2 hours. The reaction mixture was quenched with ice-cold water and extracted with EtOAc (2×15 mL). The organic layer was washed with water and dried over Na₂SO₄, filtered, concentrated to afford 4.

4 (170 mg), 1,4-dioxane (10 mL), 4 MHCl in 1,4-dioxane (1.7 mL) mixed at 0° C. then at room temperature for 5 hours. Combined batches purified by preparative HPLC gave 35 mg of 1123.

Preparation of 1113 (2R,3R,4R,5S)-1-{2-[4-(2-{[3-chloro-5-(1,2,4-oxadiazol-3-yl)phenyl]amino}ethyl)phenyl]ethyl}-2-(hydroxymethyl)piperidine-3,4,5-triol: Formic acid (5 eq), DMF (15.0 mL), HOBt (5 eq) mixed at 0° C., DIC (5 eq) added for 30 minutes, then added 1 prepared as for 1122 (400 mg) and increased temperature to room temperature for 2 hours. The reaction was quenched with ice-cold water and extracted with EtOAc (2×30 mL). The organic layer was washed with water and dried over anhydrous Na₂SO₄, filtered, concentrated to afford 400 mg of 3.

3 (400 mg), DMSO (10 mL), KOH (1.0 eq) mixed at room temperature for 16 hours. The reaction was quenched with ice-cold water and extracted with EtOAc (2×15 mL). The organic layer was washed with water and dried over Na₂SO₄, filtered, concentrated, and purified on silica [3% EtOAc in hexane] to afford 230 mg of 4.

4 (200 mg), MeOH (6 mL) mixed at 0° C., 4.0 M HCl in 1,4-dioxane (2 mL) added and increased to room temperature for 1 hour. Volatiles were removed to afford 200 mg of material which after preparative HPLC purification yielded 50.0 mg of 1113.

### Example 48: Synthesis of Compound 1124 ((2R,3R,4R,5S)-1-(6-{[3-(dimethoxymethyl)-5-(pyrimidin-2-yl) phenyl]amino}hexyl)-2-(hydroxymethyl)piperidine-3,4,5-triol and 1129 3-(pyrimidin-2-yl)-5-({6-[(2R,3R,4R,5S)-3,4,5-trihydroxy-2-(hydroxymethyl)piperidin-1-yl]hexyl}amino)benzaldehyde)

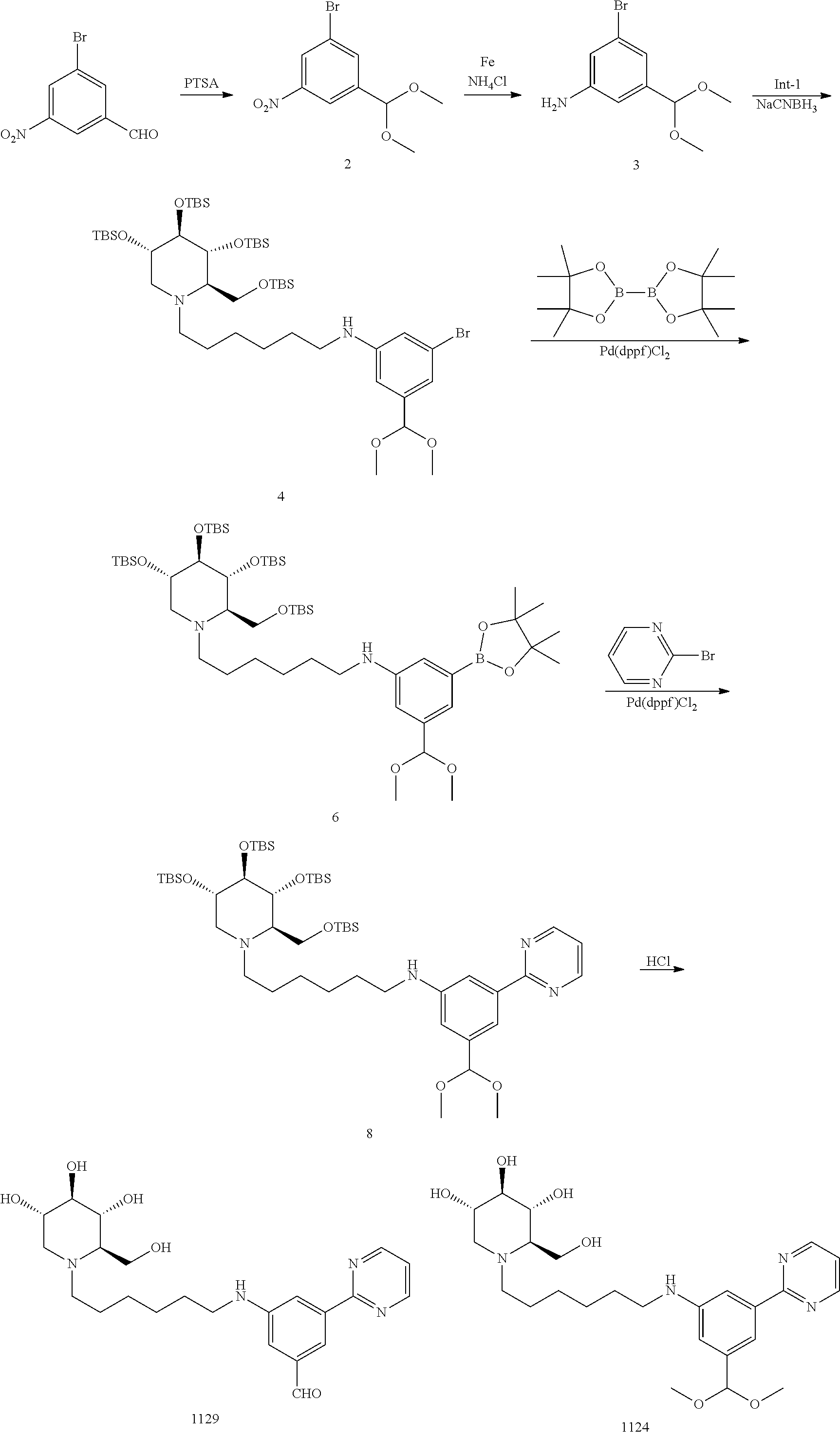

3-Bromo-5-nitro-benzaldehyde (5 g) toluene (50 mL), trimethyl orthoformate (2.0 eq), para-toluenesulfonic acid (cat.) heated to 130° C. for 20 hours. Solvent was removed. Residue was diluted with water and extracted with EtOAc. Combined organic layers was washed with brine solution, dried over anhydrous Na₂SO₄, and concentrated, and purified by chromatography [5% EtOAc/hexane] to afford 5.0 g of 2 as colorless syrup.

2 (5 g), EtOH:water (5:2, 70 mL), Iron powder (5 eq), NH₄Cl (3.5 eq) heated at reflux for 1 hour. Solvent was removed. Residue was diluted with EtOAc, filtered through CELITE, washed the CELITE bed with water. The organic layer was separated, washed with brine solution, dried over anhydrous Na₂SO₄, concentrated. Material was purified on silica [100-200 mesh, 15% EtOAc/hexane] to afford 3.5 g of 3 as yellow thick syrup.

Mixed 3 (1.0 eq), Int-1 (3.0 g), MeOH (30 mL), AcOH (cat., 0.5 mL) for 5 minutes, NaCNBH₃ (1.5 eq) added then maintained at room temperature for 16 hours. Solvent was removed. Residue was diluted with water and extracted with EtOAc. The combined organic layers were washed with brine solution, dried over Na₂SO₄, filtered, concentrated. Material was purified on silica [100-200 mesh, 15% EtOAc/hexane] to afford 1.5 g of 4 as colorless syrup.

4 (1.0 g), bis(pinacolato) diboron (1.2 eq), KOAc (3.0 eq), Pd(dppf)Cl₂ (0.1 eq), 1,4-dioxane (20 mL) degassed 15 minutes N₂, maintained at 100° C. for 16 hours. Reaction mass was diluted with EtOAc, filtered through CELITE, washed the CELITE bed with EtOAc and water. Separated the organic layer and washed with brine solution. The organic layer was dried over anhydrous Na₂SO₄ and concentrated to obtain 1.0 g of 6.

6 (700 mg), 2-bromopyrimidine (1.1 eq), Na₂CO₃ (3.0 eq), Pd(dppf)Cl₂ (0.1 eq), toluene:EtOH:water (1:1:1, 6 mL) degassed 15 minutes N₂, maintained at 90° C. for 16 hours. Reaction was diluted with water and extracted with EtOAc. The combined organic layers were washed with brine solution, dried over anhydrous Na₂SO₄, concentrated, and purified on silica [15% EtOAc/hexane] to afford 300 mg of 8 as pale brown thick syrup.

8 (280 mg), methanol (7.0 mL), 4.0 M HCl in 1,4-dioxane (2.2 mL) at 0° C., stirred at room temperature 36 hours. Purification by preparative HPLC gave 12 mg of 1124.

Alternatively, 8 (150 mg), methanol (1.5 mL), 4.0 M HCl in dioxane (3.0 mL) addition at 0° C., then maintained at room temperature for 36 hours. This material (120 mg) was taken into 2 mL DCM and added 0.1 mL TFA at 0° C. for 2 hours at to complete deprotection. Preparative HPLC purification afforded 30 mg of 1129.

### Example 49: Synthesis of Compounds 1126 (N,N-dimethyl-3-(pyrimidin-2-yl)-5-({6-[(2R,3R,4R,5S)-3,4,5-trihydroxy-2-(hydroxymethyl)piperidin-1-yl]hexyl}amino)benzamide), 1128 ((2R,3R,4R,5S)-2-(hydroxymethyl)-1-(6-{[3-methanesulfonyl-5-(pyrimidin-2-yl)phenyl]amino}hexyl)piperidine-3,4,5-triol), 1130 (3-(pyrimidin-2-yl)-5-({6-[(2R,3R,4R,5S)-3,4,5-trihydroxy-2-(hydroxymethyl)piperidin-1-yl]hexyl}amino)benzonitrile), 1131 ((2R,3R,4R,5S)-2-(hydroxymethyl)-1-(6-{[3-(pyrimidin-2-yl)-5-(trifluoromethyl)phenyl]amino}hexyl)piperidine-3,4,5-triol), and 1118 (3-(pyrimidin-2-yl)-5-({6-[(2R,3R,4R,5S)-3,4,5-trihydroxy-2-(hydroxymethyl)piperidin-1-yl]hexyl}amino) benzoic acid)

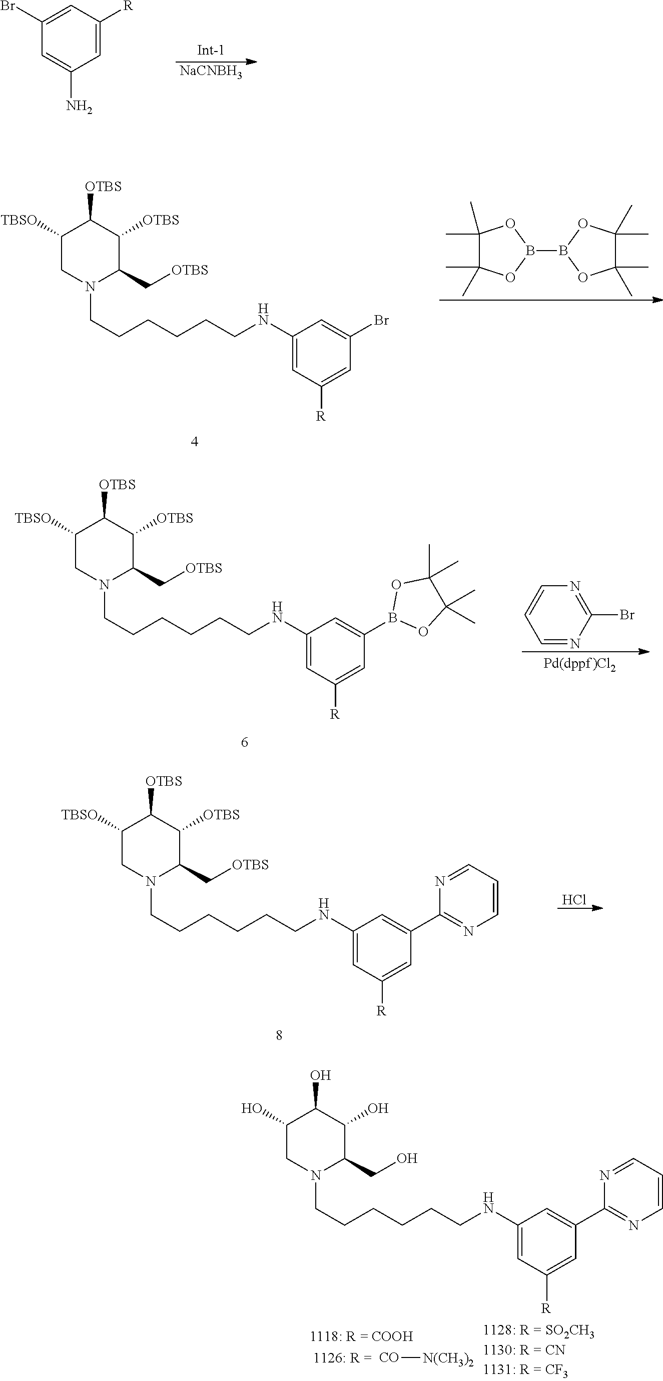

Preparation of 1126 N,N-dimethyl-3-(pyrimidin-2-yl)-5-({6-[(2R,3R,4R,5S)-3,4,5-trihydroxy-2-(hydroxymethyl)piperidin-1-yl]hexyl}amino)benzamide: 3-Bromo-5-(dimethylacetamido)aniline (300 mg), Int-1 (Example 1, 1.2 eq), MeOH (5 mL), AcOH (cat.), NaCNBH₃ (1.5 eq) mixed at room temperature for 16 hours. The solvent was concentrated and residue purified on silica [15% EtOAc/hexane] to give 400 mg of 4 as colorless syrup.

4 (350 mg), bis(pinacolato) diboron (2.2 eq), KOAc (3.0 eq), Pd(dppf)Cl₂ (0.1 eq), 1,4-dioxane (2 mL) degassed 15 minutes N₂, mixed at 90° C. for 16 hours. The reaction mass was poured into water (40 mL) and extracted with EtOAc (2×40 mL). Combined organic layers were dried over Na₂SO₄ and concentrated to obtain 440 mg of 6.

2-bromopyrimidine (60 mg), 6 (440 mg), Na₂CO₃ (3.0 eq), toluene:EtOH:water (1:1:1, 6 mL), Pd(dppf)Cl₂ (0.1 eq), degassed 15 minutes N₂, mixed at 90° C. for 16 hours. The reaction mass was poured into water (40 mL) and extracted with EtOAc (2×40 mL). Combined organic layers were dried over Na₂SO₄ and concentrated. Residue was purified on silica [25% EtOAc/hexane] to afford 110 mg of 8 as colorless syrup.

8 (110 mg), 4.0 HCl in 1,4-dioxane (1 mL), MeOH (3 mL) maintained at room temperature for 16 hours. Solvents were removed, and material purified by preparative HPLC to afford 10 mg of 1126 as off white solid.

Preparation of 1128 (2R,3R,4R,5S)-2-(hydroxymethyl)-1-(6-{[3-methanesulfonyl-5-(pyrimidin-2-yl)phenyl]amino}hexyl)piperidine-3,4,5-triol: Mixed 3-bromo-5-(methylsulfonyl)aniline (0.8 eq), Int-1 (1.0 g), MeOH (20 mL), AcOH (cat), NaCNBH₃ (1.5 eq) at room temperature. After 16 hours solvent was removed and diluted with saturated NaHCO₃ and extracted with EtOAc (2×75 mL). The organic layer was dried over anhydrous Na₂SO₄ and concentrated, and purified on silica [30% EtOAc:hexane] to afford 4 (400 mg) as colorless thick syrup.

4 (400 mg), 1,4-dioxane (25 mL), bis(pinacolato)diborane (1.5 eq), KOAc (3.0 eq) was degassed with N₂ for 10 minutes, Pd(dppf)Cl₂ (0.2 eq) added and maintained at 100° C. After 16 hours solvent was removed and diluted with water and extracted with EtOAc (2×50 mL). The organic layer was dried over anhydrous Na₂SO₄ concentrated to afford 400 mg of 6.

6 (400 mg), 2-bromopyrimidine (1.0 eq), toluene:EtOH:water (21 mL), Na₂CO₃ (3.0 eq), degassed with N₂ for 10 minutes, Pd(dppf)Cl₂ (0.1 eq) added then increased to 80° C. After 16 hours solvent was removed and diluted with water and extracted with EtOAc (2×50 mL). The organic layer was dried over anhydrous Na₂SO₄ concentrated, and purified on silica [30% EtOAc:hexane] to afford 150 mg of 8 as thick syrup.

8 (150 mg), MeOH (4 mL), 4.0 M HCl in 1,4-dioxane (1.5 mL) mixed at room temperature for 7 hours. Solvent was removed. Purification by preparative HPLC provided 26.0 mg of 1128.

Preparation of 1130 3-(pyrimidin-2-yl)-5-({6-[(2R,3R,4R,5S)-3,4,5-trihydroxy-2-(hydroxymethyl)piperidin-1-yl]hexyl}amino)benzonitrile and 1118 3-(pyrimidin-2-yl)-5-({6-[(2R,3R,4R,5S)-3,4,5-trihydroxy-2-(hydroxymethyl)piperidin-1-yl]hexyl}amino) benzoic acid: Mixed 3-bromo-5-cyanoaniline (1.2 eq), Int-1 (2 g), MeOH (10 mL), acetic acid (cat.) at 0° C., NaCNBH₃ (1.5 eq) added then increased to room temperature. After 16 hours the reaction was concentrated, the residue was diluted with EtOAc (50 mL), organic layer was washed with water (2×50 mL) followed by brine, dried over anhydrous Na₂SO₄ and concentrated. Product was purified on silica [15:85 EtOAc:hexane] to obtain 970 mg of 4.

4 (890 mg), bis(pinacolato) diboron (2.2 eq), KOAc (3.0 eq), Pd(dppf)Cl₂ (0.1 eq), 1,4-dioxane (10 mL) degassed 15 minutes N₂, reacted at 90° C. After 16 hours removed solvent, diluted with EtOAc (20 mL), washed with water (2×10 mL) followed by brine, dried over anhydrous Na₂SO₄, then concentrated to obtain 900 mg of 6.

6 (900 mg), 2-bromopyrimidine (1.1 eq), Na₂CO₃ (3.0 eq), Pd(dppf)Cl₂ (0.1 eq), toluene:EtOH:water (1:1:1, 15 mL) degassed 15 minutes N₂, reacted at 90° C. for 18 hours. Reaction was diluted with 200 mL EtOAc then the organic layer was washed with water (2×25 mL), brine, and dried over anhydrous Na₂SO₄ and concentrated. Material was purified on silica [60-120 silica mesh, 15:85 EtOAc:hexane] to obtain 500 mg of 8.

8 (100 mg), 4.0 M HCl in 1,4-dioxane (1 mL), 1,4-dioxane (4 mL) mixed at room temperature for 2 hours. Volatile solvents were removed, residue was basified with saturated NH₄HCO₃ and residual solvent was removed. Combined batches were purified by preparative HPLC to afford 18.0 mg of 1130.

In a separate reaction, 8 (220 mg), 0.4 M HCl in 1,4-dioxane (2 mL), DCM:MeOH (1.5 mL:0.5 mL) was stirred at room temperature for 2 hours to hydrolyze both the protecting groups and the nitrile. After preparative HPLC purification 20 mg of 1118 was obtained.

Preparation of 1131 (2R,3R,4R,5S)-2-(hydroxymethyl)-1-(6-{[3-(pyrimidin-2-yl)-5-(trifluoromethyl)phenyl]amino}hexyl)piperidine-3,4,5-triol: Mixed Int-1 (1.2 eq), MeOH (5 mL), 3-bromo-5-trifluoromethylaniline (500 mg), AcOH (cat), NaCNBH₃ (1.5 eq) at room temperature for 16 hours. The solvent was concentrated. The residue was purified on silica [3% EtOAc/hexane] to afford 500 mg of 4 as colorless syrup.

4 (500 mg), bis(pinacolato) pyridine (2.2 eq), KOAc (3.0 eq), Pd(dppf)Cl₂ (0.1 eq), 1,4-dioxane (5 mL) degassed 15 minutes N₂, heated at 90° C. for 16 hours. The reaction mass was poured into water (30 mL) and extracted with EtOAc (2×30 mL). Combined organic layers were dried over Na₂SO₄ and concentrated to obtain 750 mg of 6.

2-Bromopyrimidine (120 mg), 6 (750 mg), Na₂CO₃ (3.0 eq), Pd(dppf)Cl₂ (0.1 eq), toluene:EtOH:water (1:1:1, 6 mL) degassed 15 minutes N₂, maintained at 90° C. for 16 hours. The reaction mass was poured into water (30 mL) and extracted with EtOAc (2×30 mL). Combined organic layers were dried over Na₂SO₄ and concentrated. Material was purified on silica [8% EtOAc/hexane] to afford 170 mg of 8 as colorless syrup.

8 (170 mg), 4.0 M HCl in 1,4-dioxane (2 mL), MeOH (4 mL) mixed at room temperature for 16 hours. Solvents were removed, and purified by preparative HPLC to afford 15 mg of 1131 as off-white solid.

### Example 50: Synthesis of Compound 1133 (3-methoxy-5-{[2-(4-{2-[(2R,3R,4R,5S)-3,4,5-trihydroxy-2-(hydroxymethyl)piperidin-1-yl]ethyl}phenyl)ethyl]amino}benzonitrile)

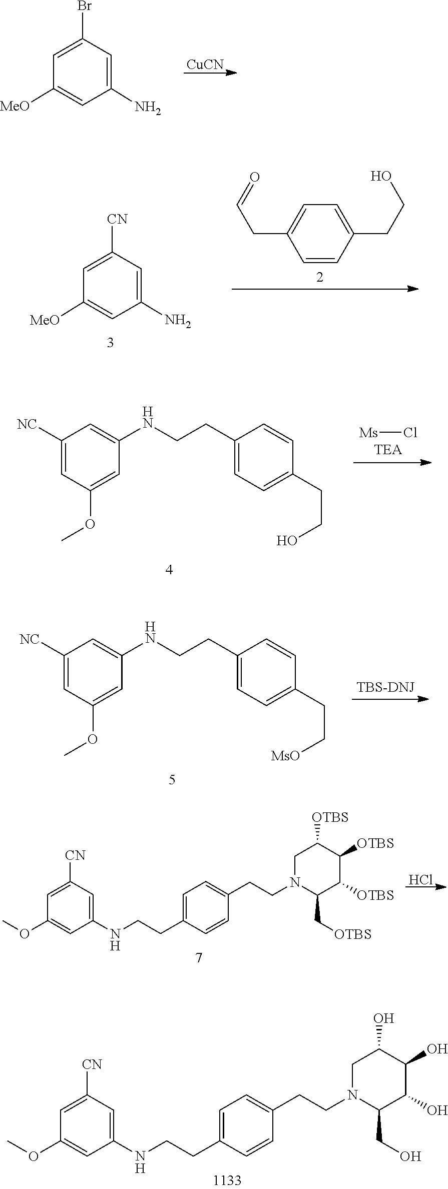

3-Bromo-5-methoxyaniline (4.0 g), NMP (40 mL), CuCN (1.3 eq) were added at room temperature, then heated at 200° C. for 6 hours. The reaction was diluted with EtOAc and saturated NaHCO₃, filtered, filtrate washed with brine then extracted with EtOAc (2×5 mL). The organic layer was dried over Na₂SO₄, filtered, concentrated, and purified on silica [1% MeOH/DCM] to afford 2.0 g of 3 as orange solid.

To prepare 2, DCM (300 mL), 1,4-bis(2-hydroxyethyl)benzene (3.0 g) and DMP (1.0 eq) were mixed at 0° C. for 15 minutes. The reaction was quenched with ice-cold water, extracted with DCM (2×100 mL), the organic layer was dried over anhydrous Na₂SO₄ and concentrated to afford 3.0 g of 2.

2 (3.0 g), MeOH:DCM (2:1, 30 mL), 3 (1.0 eq), AcOH (cat.), 0° C., NaCNBH₃ (1.5 eq), added at 0° C. then increased to room temperature for 16 hours. The reaction was concentrated then residue was quenched with ice-cold water and extracted with EtOAc (2×100 mL). The organic layer was dried over Na₂SO₄, filtered, concentrated, and purified on silica [1-2% DCM in MeOH] to afford 2.5 g of 4.

4 (2.5 g), DCM (50 mL), TEA (3.0 eq), Ms-Cl (1.5 eq) mixed at 0° C. for 10 minutes. The reaction was quenched with ice-cold water and extracted with DCM (2×100 mL). The organic layer was dried over Na₂SO₄, filtered, concentrated, and purified on silica [40-50% EtOAc in hexane] to afford 2.5 g of 5.

5 (2.5 g), TBS-DNJ (1.0 eq), ACN (30 mL), K₂CO₃ (3.0 eq) added at room temperature then increased to 90° C. After 36 hours the reaction mixture was cooled to room temperature and concentrated. The residue was quenched with ice-cold water and extracted with EtOAc (2×50 mL). The organic layer was dried over Na₂SO₄, filtered, concentrated, and purified on silica [⁶-8% EtOAc in hexane] to afford 2.0 g of 7.

7 (200 mg), 4.0 M HCl in 1,4-dioxane (2.0 mL), 1,4-dioxane (10 mL) mixed at room temperature for 8 hours. The volatiles solvents were removed at room temperature and reaction mixture was neutralized with aqueous NH₄HCO₃ and co-distilled with MeOH. After purification by preparative HPLC 20 mg of 1133 was obtained.

### Example 51: Synthesis of Compounds 1134 (3-chloro-5-{[2-(4-{2-[(2R,3R,4R,5S)-3,4,5-trihydroxy-2-(hydroxymethyl)piperidin-1-yl]ethyl}phenyl)ethyl]amino}benzonitrile), 1117 (3-chloro-5-{[2-(4-{2-[(2R,3R,4R,5S)-3,4,5-trihydroxy-2-(hydroxymethyl)piperidin-1-yl]ethyl}phenyl)ethyl]amino}benzoic acid and 1117 (3-chloro-5-{[2-(4-{2-[(2R,3R,4R,5S)-3,4,5-trihydroxy-2-(hydroxymethyl)piperidin-1-yl]ethyl}phenyl)ethyl]amino}benzoic acid) and 1116 (methyl 3-chloro-5-{[2-(4-{2-[(2R,3R,4R,5S)-3,4,5-trihydroxy-2-(hydroxymethyl)piperidin-1-yl]ethyl}phenyl)ethyl]amino}benzoate)

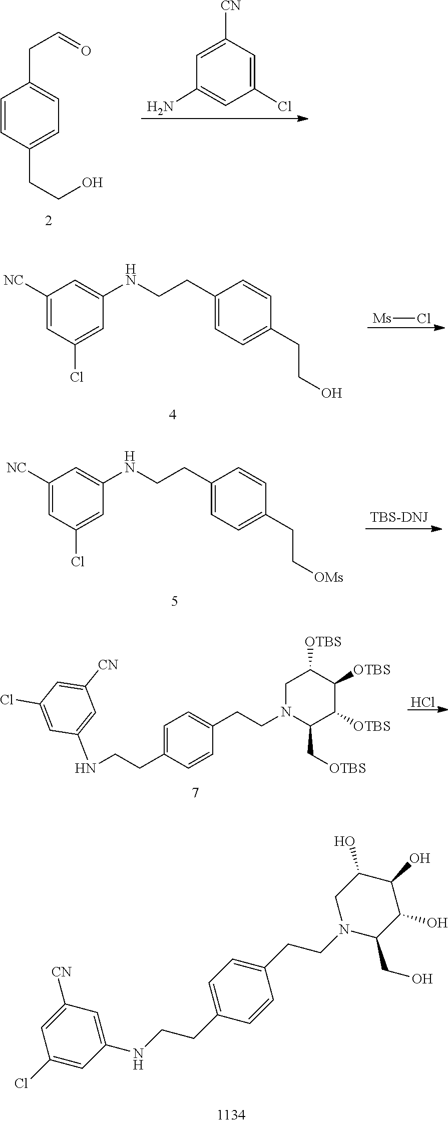

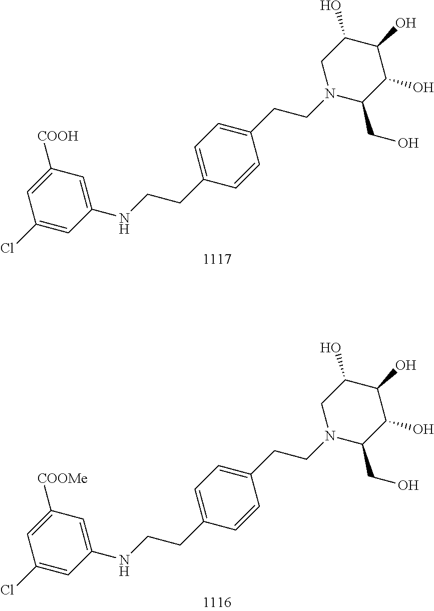 [SMILES: O=C(O)c1cc(Cl)cc(NCCc2ccc(CCN3C[C@H](O)[C@@H](O)[C@H](O)[C@H]3CO)cc2)c1 ; COC(=O)c1cc(Cl)cc(NCCc2ccc(CCN3C[C@H](O)[C@@H](O)[C@H](O)[C@H]3CO)cc2)c1]

2 (Example 50, 5 g), DCM/MeOH (1:1, 80 mL), 3-chloro-5-cyanoaniline (1 eq), AcOH (0.1 mL) mixed at 0° C., NaCNBH₃ (1.5 eq) added and increased to room temperature. After 16 hours the reaction was concentrated, residue was quenched with ice-cold water and extracted with EtOAc (2×10 mL). The organic layer was dried over Na₂SO₄, filtered, concentrated, and purified on silica [40-50% EtOAc in hexane] to afford 2.0 g of 4.

4 (2 g), DCM (50 mL), TEA (3.0 eq), MsCl (1.5 eq) mixed at 0° C. for 10 minutes. The reaction was quenched with ice-cold water and extracted with DCM (2×50 mL). The organic layer was dried over Na₂SO₄, filtered, concentrated, and purified on silica [30-40% EtOAc in hexane] to afford 2.1 g of 5.

5 (2.1 g), TBS-DNJ (1.0 eq), ACN (50 mL), K₂CO₃ (3.0 eq) reacted at 90° C. for 48 hours. The reaction mixture was cooled to room temperature and concentrated. The residue was quenched with ice-cold water, extracted with EtOAc (2×100 mL), organic layer was dried over Na₂SO₄, filtered, concentrated, and purified on silica [3% EtOAc in hexane] to afford 2.5 g of 7.

Preparation of 1134 3-chloro-5-{[2-(4-{2-[(2R,3R,4R,5S)-3,4,5-trihydroxy-2-(hydroxymethyl)piperidin-1-yl]ethyl}phenyl)ethyl]amino}benzonitrile: 7 (100 mg), MeOH/DCM (2 mL/2 mL), 4.0 M HCl in dioxane (1 mL) mixed at 0° C. then increased to room temperature for 1 hour. Reaction mass was basified with aq. saturated NH₄HCO₃, concentrated. Purification by preparative HPLC yielded 11 mg of 1134.

Preparation of 1117 3-chloro-5-{[2-(4-{2-[(2R,3R,4R,5S)-3,4,5-trihydroxy-2-(hydroxymethyl)piperidin-1-yl]ethyl}phenyl)ethyl]amino}benzoic acid and 1116 methyl 3-chloro-5-{[2-(4-{2-[(2R,3R,4R,5S)-3,4,5-trihydroxy-2-(hydroxymethyl)piperidin-1-yl]ethyl}phenyl)ethyl]amino}benzoate: In a separate reaction, 7 (250 mg), MeOH/DCM (1 mL/1 mL), 4.0M HCl in dioxane (2 mL) mixed at 0° C. then increased to room temperature for 4 hours to hydrolyze the nitrile. Reaction mass was concentrated. After preparative HPLC purification recovered 10 mg of 1117 (benzoic acid) and 20 mg of 1116 (methanoate ester).

### Example 52: Synthesis of Compound 1136 ((2R,3R,4R,5S)-1-{6-[(4-cyclopropyl-2-nitrophenyl)amino]hexyl}-2-(hydroxymethyl)piperidine-3,4,5-triol)

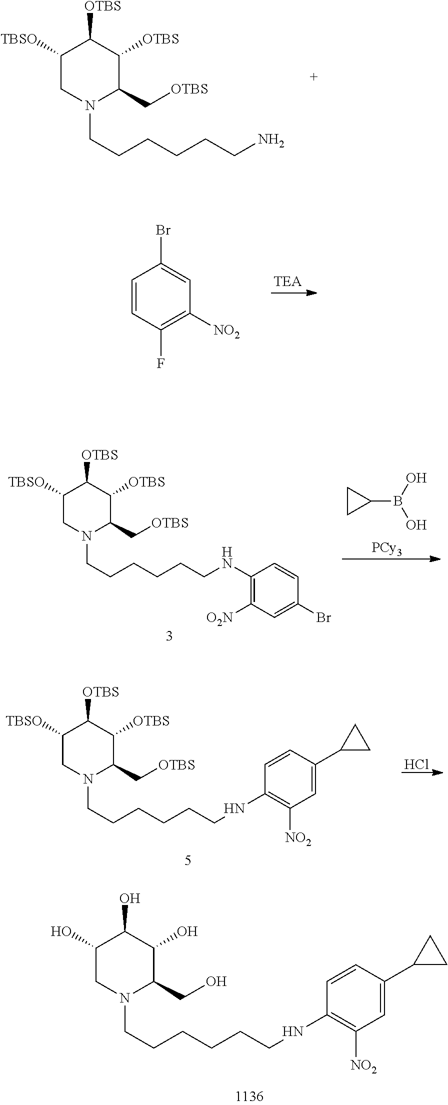

Int-4 (1 g), 1,4-dioxane (10 mL), 2-fluoro-5-bromonitrobenzene (1 eq), TEA (4 eq) mixed at room temperature, increased to 100° C. After 5 hours, solvent was removed and residue was diluted with water and extracted with EtOAc. The organic layer was dried over anhydrous Na₂SO₄ and concentrated, and purified on silica [4% EtOAc:hexane] to afford 550 mg of 3.

3 (250 mg), toluene:water (12 ml, 20:1), cyclopropyl boronic acid (1.3 eq), K₃PO₄ (3.0 eq), tricyclohexylphosphine (PCy₃, 0.1 eq) was degassed for 20 minutes, Pd(OAc)₂ (0.05 eq) added then heated at 100° C. for 5 hours. The reaction mass was diluted with water and extracted with EtOAc. The organic layer was washed with brine solution, dried over anhydrous Na₂SO₄, concentrated, and purified on silica [3% EtOAc in hexane] to afford 200 mg of 5 as orange thick syrup.

5 (200 mg), MeOH (2 mL), cooled to 0° C., HCl in 1,4-dioxane (5 mL) added then maintained at room temperature for 16 hours. The reaction was concentrated. Combined batches purified by preparative HPLC afforded 30 mg of 1136.

### Example 53: Synthesis of Compound 1138 ((2R,3R,4R,5S)-2-(hydroxymethyl)-1-[6-({3-nitro-[1,1′-biphenyl]-4-yl}amino)hexyl]piperidine-3,4,5-triol)

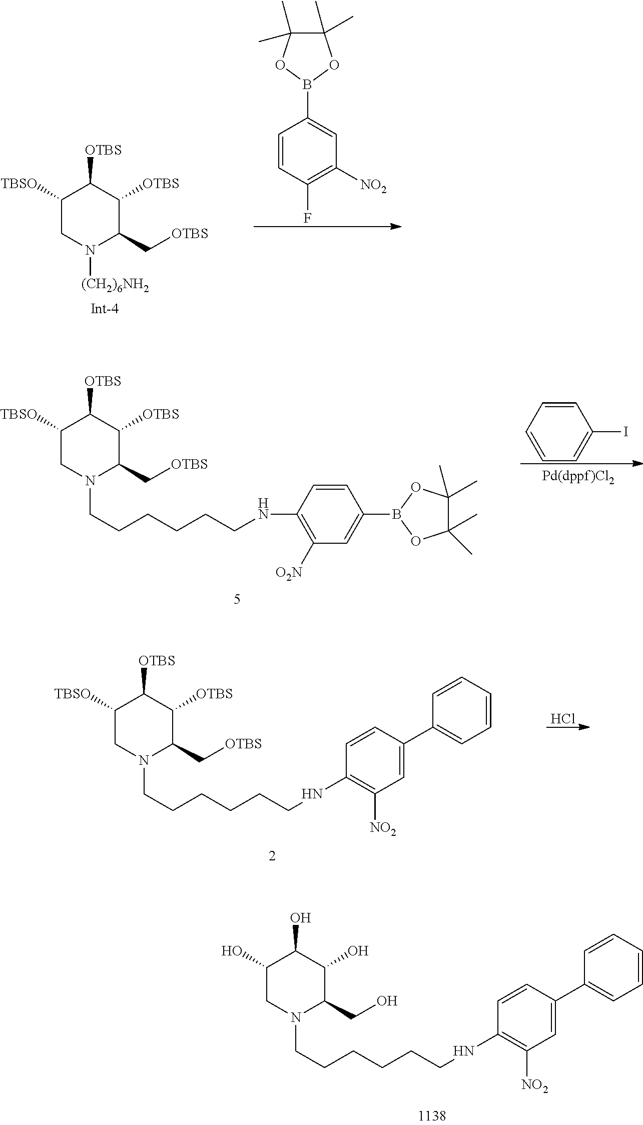

Int-4 (6.0 g, Example 4), 1, 4-dioxane (200 mL), 4-fluoro-3-nitro-phenylboronate (0.8 eq), TEA (3.0 eq) reacted at 100° C. for 4 hours. The reaction was concentrated to obtain residue. The obtained residue was diluted with water and extracted with EtOAc. The organic layer was dried over Na₂SO₄, filtered, concentrated to afford 6.5 g of 5.

5 (500 mg), toluene:EtOH:water (30 ml, 1:1:1), iodobenzene (1.1 eq), Na₂CO₃ (3.0 eq), degassed for 5 minutes, Pd(dppf)Cl₂ (0.1 eq) added and maintained at 80° C. for 16 hours. Solvent was removed and residue diluted with water. The aqueous layer was extracted with EtOAc and dried over anhydrous Na₂SO₄ and concentrated, and purified on silica [5% EtOAc in hexane] to afford 300 mg of 2 as orange colored thick syrup.

2 (300 mg), MeOH (15 mL) mixed at 0° C., added HCl in 1,4-dioxane (1 mL), maintained 2 hours at room temperature, solvent was removed, purification by preparative HPLC gave 79 mg of 1138.

### Example 54: Synthesis of Compound 1140 ((2R,3R,4R,5S)-1-[2-(3-{2-[(4-azido-2-nitrophenyl)amino]ethyl}-5-bromophenyl)ethyl]-2-(hydroxymethyl)piperidine-3,4,5-triol)

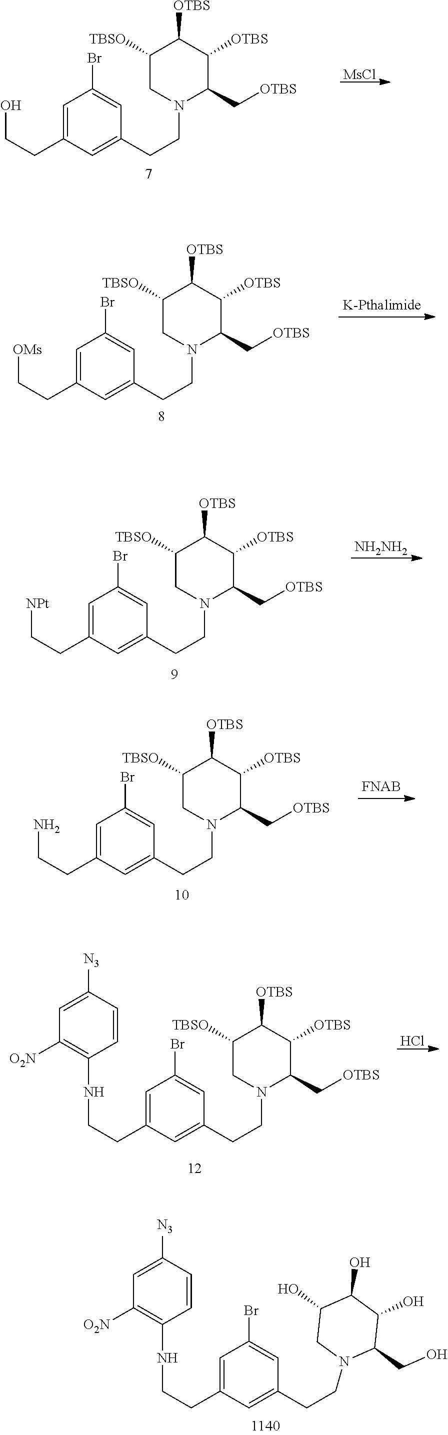

7 (2.2 g, Example 45) in DCM (40 mL) was mixed at 0° C., added mesyl chloride (1.1 eq), TEA (3 eq), maintained 1 hour. Cold water was added then extracted with DCM. The organic layer was washed with brine, dried over anhydrous Na₂SO₄ and concentrated. Material was purified on silica [10% EtOAc/hexane] to afford 1.5 g of 8 as colorless liquid.

To 8 (1.5 g) in DMF (15 mL) at room temperature added potassium phthalimide (1.2 eq), warmed to 80° C. for 36 hours. Cooled, diluted with water, extracted with EtOAc. Organic layers were washed with brine, dried over anhydrous Na₂SO₄ and concentrated. Material was purified on silica [10% EtOAc/hexane] to afford 600 mg of 9 as colorless liquid.

9 (600 mg), EtOH (10.0 mL), hydrazine hydrate (75% solution, 5.0 eq) maintained at room temperature for 16 hours. Solvent was removed and residue diluted with water and extracted with EtOAc. Organic layers were washed with brine, dried over anhydrous Na₂SO₄, filtered, concentrated to provide 400 mg of 10.

10 (300 mg), 1,4-dioxane (15 mL), Et₃N (3 eq), FNAB (1.0 eq) mixed and heated at 100° C. for 20 hours. Volatiles were removed. Residue was purified on silica [3% EtOAc/hexane] to afford 120 mg of 12 as colorless liquid.

12 (120 mg), MeOH (3 mL), 4.0 M HCl in 1,4-dioxane (1.5 mL) mixed at 0° C. then stirred at room temperature for 16 hours. Volatiles were removed. Combined batches after preparative HPLC purification yielded 12 mg of 1140.

### Example 55: Synthesis of Compounds 1141 ((2R,3R,4R,5S)-2-(hydroxymethyl)-1-[6-({3-methoxy-5-[(oxolan-3-yl)methyl]phenyl}amino)hexyl]piperidine-3,4,5-triol), 1145 ((2R,3R,4R,5S)-1-{6-[(3-benzyl-5-methoxyphenyl)amino]hexyl}-2-(hydroxymethyl)piperidine-3,4,5-triol), 1146 ((2R,3R,4R,5S)-2-(hydroxymethyl)-1-(6-{[3-methoxy-5-(oxolan-2-yl)phenyl]amino}hexyl)piperidine-3,4,5-triol), 1147 ((2R,3R,4R,5S)-1-(6-{[3-(4,5-dihydrofuran-2-yl)-5-methoxyphenyl]amino}hexyl)-2-(hydroxymethyl)piperidine-3,4,5-triol), 1148 ((2R,3R,4R,5S)-1-{6-[(3-cyclohexyl-5-methoxyphenyl)amino]hexyl}-2-(hydroxymethyl)piperidine-3,4,5-triol), 1149 ((2R,3R,4R,5S)-1-(6-{[3-(cyclohex-1-en-1-yl)-5-methoxy-phenyl]amino}hexyl)-2-(hydroxymethyl)piperidine-3,4,5-triol), 1150 ((2R,3R,4R,5S)-2-(hydroxymethyl)-1-[6-({5-methoxy-[1,1′-biphenyl]-3-yl}amino)hexyl]piperidine-3,4,5-triol), 1197 (2R,3R,4R,5S)-2-(hydroxymethyl)-1-(6-{[3-methoxy-5-(pyrazin-2-yl)phenyl]amino}hexyl)piperidine-3,4,5-triol), 1216 ((2R,3R,4R,5S)-2-(hydroxymethyl)-1-(6-{[3-methoxy-5-(1,3-oxazol-2-yl)phenyl]amino}hexyl)piperidine-3,4,5-triol), 1217 ((2R,3R,4R,5S)-2-(hydroxymethyl)-1-(6-{[3-methoxy-5-(pyrimidin-2-yl)phenyl]amino}hexyl)piperidine-3,4,5-triol), and 1219 ((2R,3R,4R,5S)-2-(hydroxymethyl)-1-(6-{[3-(1H-imidazol-2-yl)-5-methoxyphenyl]amino}hexyl)piperidine-3,4,5-triol)

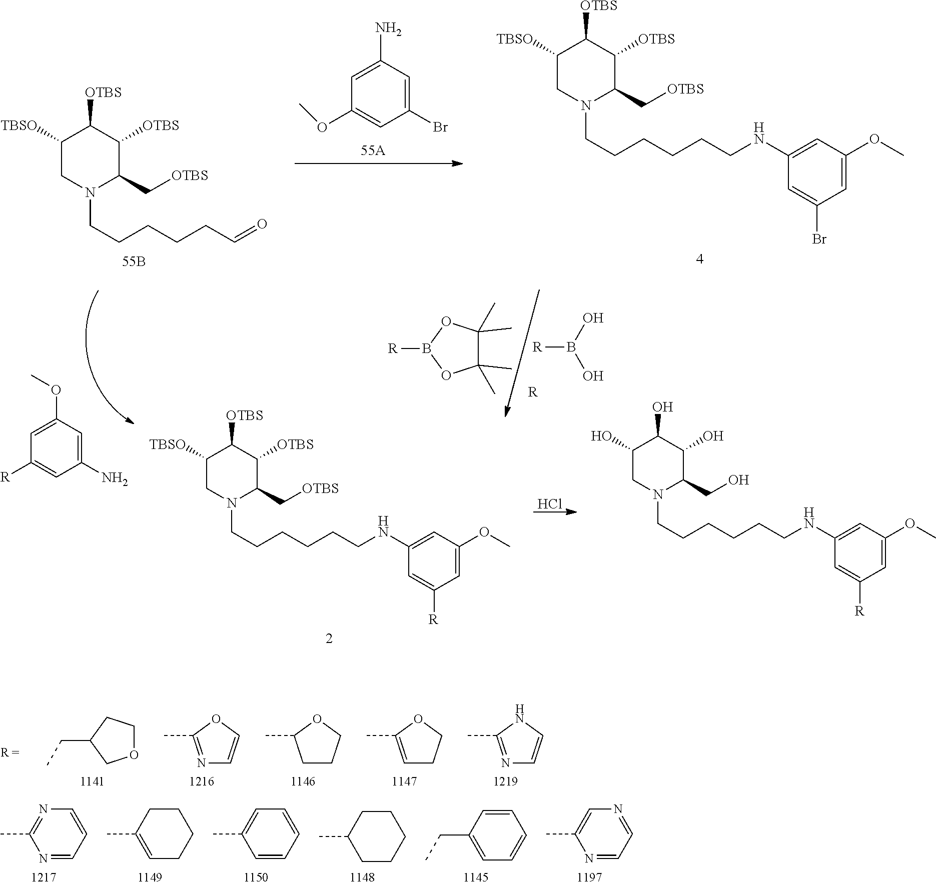

Mixed Int-1 (Example 1, 10.0 g), 3-bromo-5-methoxyaniline (1.0 eq), MeOH (100 mL), AcOH (cat.), added NaCNBH₃ (1.5 eq), maintained at room temperature 16 hours. The reaction was concentrated. The residue was quenched with ice-cold water and extracted with EtOAc (2×100 mL). The organic layer was dried over Na₂SO₄, filtered, concentrated. The residue was purified on silica [8% EtOAc/hexane] to afford 3.8 g of 4.

Preparation of 1141 (55A) (2R,3R,4R,5S)-2-(hydroxymethyl)-1-[6-({3-methoxy-5-[(oxolan-3-yl)methyl]phenyl}amino)hexyl]piperidine-3,4,5-triol: Reacted 4 (150 mg), ((tetrahydrofuran-3-yl)methyl)-boronate (1.0 eq), 1,4-dioxane:water (3:1, 8 mL), K₂CO₃ (3.0 eq), Pd(dppf)₂Cl₂ (0.1 eq) at 100° C. 16 hours after degassing 15 minutes under N₂. The reaction was diluted with water (20 mL), extracted with EtOAc (2×20 mL), the organic layer dried over anhydrous Na₂SO₄ and concentrated. 2 was purified on silica [8-10% EtOAc in hexane].

To 2 (60 mg) in MeOH (3 mL) at 0° C. added 4.0 M HCl in 1,4-dioxane (0.3 mL) and increased to room temperature for 2 hours. Solvent was removed. After purification by preparative HPLC, 4 mg of 1141 was obtained.

Preparation of 1145 (55A) (2R,3R,4R,5S)-1-{6-[(3-benzyl-5-methoxyphenyl)amino]hexyl}-2-(hydroxymethyl)piperidine-3,4,5-triol: 4 (500 mg), 1,4-dioxane:water (3:1, 5 mL), benzylboronate (1.5 eq), Na₂CO₃ (3.0 eq), Pd(pph₃)₄ (0.1 eq) reacted at 100° C. in microwave reactor for 1 hour after degassing 15 minutes under N₂. The reaction was diluted with water and extracted with EtOAc (2×5 mL). The organic layer was dried over Na₂SO₄, filtered, concentrated, and purified on silica [5% EtOAc:hexane] to afford 100 mg of 2.

2 (100 mg), 4M HCl in 1,4-dioxane (1.0 mL), MeOH (2.0 mL) mixed at 0° C. and increased to room temperature. After 16 hours reaction mass was concentrated. The residue was purified by preparative HPLC to afford 10 mg of 1145.

Preparation of 1146 (55A) (2R,3R,4R,5S)-2-(hydroxymethyl)-1-(6-{[3-methoxy-5-(oxolan-2-yl)phenyl]amino}hexyl)piperidine-3,4,5-triol: 4 (500 mg), DMF (4.5 mL) was mixed with 2,3-dihydrofuran (10.0 eq), K₂CO₃ (2.0 eq), degassed for 5 minutes under Ar, then TPP (0.2 eq) and Pd(OAc)₂ (0.1 eq) added at room temperature then increased to 110° C. After 10 hours quenched with ice-cold water and extracted with EtOAc (2×50 mL). The organic layer was washed with water (10 mL), brine (10 mL), dried over Na₂SO₄, filtered, concentrated, and purified on silica [8-10% EtOAc in hexane].

2 (220 mg), EtOAc:MeOH (1:1, 5 mL), 10% Pd/C (100 mg, 50% wet) maintained under mild H₂ pressure (balloon) for 6 hours. The reaction mass was filtered through a pad of CELITE and the filtrate concentrated. 180 mg of the material was used in the de-protection step with 4.0 M HCl in 1,4-dioxane (1.5 mL), 1,4-dioxane (4.5 mL) at room temperature. After 6 hours volatiles were removed at room temperature. The residue was neutralized with aq. NH₄HCO₃ and co-distilled with methanol. Purification by preparative HPLC yielded 10 mg of 1146.

Preparation of 1147 (55A) (2R,3R,4R,5S)-1-(6-{[3-(4,5-dihydrofuran-2-yl)-5-methoxyphenyl]amino}hexyl)-2-(hydroxymethyl)piperidine-3,4,5-triol: Mixed 4 (500 mg), DMF (4.5 mL), 2,3-dihydrofuran (10.0 eq), K₂CO₃ (2.0 eq), degas for 5 minutes under Ar, added TPP (0.2 eq), Pd(OAc)₂ (0.1 eq) at room temperature, and increased to 110° C. After 10 hours quenched with ice-cold water and extracted with EtOAc (2×50 mL). The organic layer was washed with water (10 mL), brine (10 mL), dried over Na₂SO₄, filtered, concentrated, and purified on silica [8-10% EtOAc:hexane] to afford two fractions, with Fr-II (major) 200 mg proceeding directly to deprotection.

Fr-II (150 mg), 4.0 M HCl in 1,4-dioxane (1.5 mL), 1,4-dioxane (4.5 mL) mixed at room temperature for 6 hours. Volatiles were removed at room temperature. Residue was neutralized with aq. NH₄HCO₃ and co-distilled with methanol. Preparative HPLC purification yielded 3.2 mg of 1147.

Preparation of 1148 (55A) (2R,3R,4R,5S)-1-{6-[(3-cyclohexyl-5-methoxyphenyl) amino]hexyl}-2-(hydroxymethyl)piperidine-3,4,5-triol: Reacted 4 (500 mg), 1,4-dioxane:water (3:1, 5 mL), 1-cyclohexen-1-yl-boronate (1.5 eq), Na₂CO₃ (3.0 eq), Pd(pph₃)₄ (0.1 eq) at 100° C. in microwave for 1 hour after degassing 15 minutes under N₂. The reaction was diluted with water, extracted with EtOAc (2×5 mL), the organic layer dried over Na₂SO₄, filtered, concentrated, and purified on silica [5% EtOAc:hexane] to afford 250 mg of 2.

2 (200 mg), 10% Pd/C (50% wt/wt, 20 mg), EtOAc (10 mL) maintained under H₂ at room temperature for 6 hours. Product was combined with another batch executed on 50 mg scale and was worked up by filtration and removal of volatile solvents to obtain 200 mg of product which was used for the deprotection with 4M HCl in 1,4-dioxane (1.0 mL), MeOH (2.0 mL), mixed at 0° C. and maintained at room temperature for 16 hours. Reaction mass was concentrated, and residue purified by preparative HPLC to afford 40 mg of 1148.

Preparation of 1149 (55A) (2R,3R,4R,5S)-1-(6-{[3-(cyclohex-1-en-1-yl)-5-methoxy-phenyl]amino}hexyl)-2-(hydroxymethyl)piperidine-3,4,5-triol: Reacted 4 (500 mg), 1,4-dioxane:water (3:1, 5 mL), 1-cyclohexen-1-yl-boronate (1.5 eq), Na₂CO₃ (3.0 eq), Pd(pph₃)₄ (0.1 eq) at 100° C. in microwave reactor for 1 hour after degassing 15 minutes under N₂. Diluted with water and extracted with EtOAc (2×5 mL), dried organic layer over Na₂SO₄, filtered, concentrated, and purified on silica [5% EtOAc:hexane] to afford 250 mg of 2.

2 (150 mg), 4M HCl in 1,4-dioxane (1 mL), MeOH (2 mL) mixed at 0° C. then increased to room temperature. After 16 hours reaction mass was concentrated, and purified by preparative HPLC to afford 15 mg of 1149.

Preparation of 1150 (55A) (2R,3R,4R,5S)-2-(hydroxymethyl)-1-[6-({5-methoxy-[1,1′-biphenyl]-3-yl}amino)hexyl]piperidine-3,4,5-triol: 4 (300 mg), phenylboronic acid (1.2 eq), toluene:EtOH:water (1:1:1, 6 mL), Na₂CO₃ (3.0 eq), Pd(dppf)Cl₂ (0.1 eq) degassed 15 minutes N₂, reacted at 80° C. for 8 hours. Mix was cooled to room temperature and solvent was removed. Added water to residue and extracted with EtOAc (2×50 mL). The organic layer was washed with brine and dried over Na₂SO₄, filtered, concentrated, and material was purified on silica [7-8% EtOAc/hexane] to afford 220 mg of 2.

2 (220 mg), MeOH (6.0 mL), 4.0 M HCl in 1,4-dioxane (2.2 mL) added at 0° C. then warmed to room temperature for 4 hours. The reaction was concentrated and preparative HPLC purification of combined batches provided 6.2 mg of 1150.

Preparation of 1219 (55B) (2R,3R,4R,5S)-2-(hydroxymethyl)-1-(6-{[3-(1H-imidazol-2-yl)-5-methoxyphenyl]amino}hexyl)piperidine-3,4,5-triol: 3-Bromo-5-methoxyaniline (3.0 g) was reacted with bispinacolato diboron (1.5 eq) in 1,4 dioxane (60 mL) with Pd(dppf)Cl₂ and KOAc (3.0 eq) at 120° C. for 4 hours after degassing 15 minutes with N₂. Water was added and extracted with EtOAc. The organic layer was separated, dried over Na₂SO₄, concentrated under vacuum, and purified on silica [20-50% EtOAc/hexane] to obtain 2.1 g of the boronate.

2-Bromoimidazole (2 g) was protected by mixing in DCM (60 mL), adding TEA (1.9 mL) and (Boc)₂O (2.98 mmol) at room temperature and stirring at room temperature for 4 hours. Reaction was quenched with ice-cold water and extracted with DCM (50 mL). The organic layer was separated, dried over Na₂SO₄, filtered, concentrated, and passed through a plug of silica gel to obtain 2.8 g of the tert-butyl 2-bromo-1H-imidazole-1-carboxylate. 1.2 equivalents of this material was degassed for 20 minutes under N₂ in DME:H₂O (8.0 mL), added 3-methoxyaniline-5-boronate (1 g), Cs₂CO₃ (3.0 eq), Pd(dppf)Cl₂ (0.1 eq) degassed 15 minutes N₂, then reacted at 85° C. for 1 hour in microwave. The reaction was cooled to room temperature and volatiles evaporated. Residue was dissolved in EtOAc (50 mL), washed with water (20 mL), organic layer was dried over Na₂SO₄, filtered, concentrated. Two similar batches were combined and purified by COMBIFLASH [20-40% EtOAc/hexanes] to afford 160 mg of tert-butyl 2-(3-amino-5-methoxyphenyl)-1H-imidazole-1-carboxylate which was mixed with 1.5 eq. Int-1 (Example 1) in MeOH (7 mL) at 0° C., added NaCNBH₄ (1.5 eq), AcOH (cat.) and maintained at room temperature 16 hours. The reaction was diluted with water and extracted with EtOAc. The organic layer was dried over anhydrous Na₂SO₄, concentrated, and the residue purified on silica [40% EtOAc in hexane] to afford 500 mg of 2.

Mixed 2 (400 mg) in MeOH (10 mL) at 0° C., added 4.0M HCl in 1,4-dioxane (4 mL), maintained at room temperature for 5 hours. Solvent was removed and preparative HPLC purification provided 40 mg of 1219.

Preparation of 1216 (55B) (2R,3R,4R,5S)-2-(hydroxymethyl)-1-(6-{[3-methoxy-5-(1,3-oxazol-2-yl)phenyl]amino}hexyl)piperidine-3,4,5-triol: Reacted 3-methoxy-aniline-5-boronate (900 mg), 2-bromo-oxazole (992 mg, 1.5 eq.), toluene:EtOH:water (40 mL), Na₂CO₃ (3 eq), Pd(dppf)Cl₂ (0.1 eq) at 80° C. for 4 hours after degassing 15 minutes N₂. Cooled to room temperature, added water and extracted with EtOAc (2×50 mL). The organic layer was dried over Na₂SO₄, filtered, concentrated, and residue purified on silica [20%-40% EtOAc in hexane] to afford 401 mg of the desired aniline.

Int-1 (1.5 g, Example 1), 1 eq. 3-methoxy-5-(oxazol-2-yl)aniline, MeOH (30 mL), AcOH (cat.) mixed at 0° C., added NaCNBH₃ (1.5 eq), reacted at room temperature 16 hours. The reaction was concentrated, residue was dissolved in water (100 mL) and extracted with EtOAc (2×100 mL). The organic layer was dried over Na₂SO₄, filtered, concentrated and the material purified on silica [10-20% EtOAc in hexane] to afford 840 mg of 2.

2 (840 mg), 1,4-dioxane (20 mL), 4.0 M HCl in dioxane (5 mL) reacted at room temperature 16 hours. The reaction was concentrated and preparative HPLC purification gave 50 mg of 1216.

Preparation of 1197 (55B) (2R,3R,4R,5S)-2-(hydroxymethyl)-1-(6-{[3-methoxy-5-(pyrazin-2-yl)phenyl]amino}hexyl)piperidine-3,4,5-triol: 3-Methoxyaniline-5-boronate (1 g), 2-bromopyrazine (1 eq.), Pd(dppf)Cl₂ (0.1 eq), Na₂CO₃ (3 eq), toluene (1 mL), EtOH (1 mL) and H₂O (3 mL) degassed 15 minutes N₂, reacted at 80° C. for 2 hours. Reaction was quenched with water, diluted with EtOAc (100 mL). The organic layer was separated, washed with brine, dried over Na₂SO₄, concentrated, and the residue purified on silica [40% EtOAc:hexane] to afford 400 mg of desired aniline as colorless thick syrup.

3-methoxy-5-(pyrazin-2-yl)aniline (400 mg), MeOH (20 mL), Int-1 (1 eq), AcOH (cat.) mixed at 0° C., added NaCNBH₃ (1.5 eq), reacted at room temperature 16 hours. Reaction was diluted with water and extracted with EtOAc. The organic layer was dried over anhydrous Na₂SO₄, concentrated, and purified on silica [10% EtOAc in hexane] to afford 800 mg of 2.

Mixed 2 (400 mg), MeOH (15 mL) at 0° C. added 4.0 M HCl in 1,4-dioxane (6 mL), reacted at room temperature for 2 hours. The reaction mass was concentrated and after preparative HPLC purification yielded 35 mg of 1197.

Preparation of 1217 (55A) (2R,3R,4R,5S)-2-(hydroxymethyl)-1-(6-{[3-methoxy-5-(pyrimidin-2-yl)phenyl]amino}hexyl)piperidine-3,4,5-triol: 4 was converted to the boronate ester by mixing 1.25 g of 4, bis(pinacolato) diboron (1.5 eq), Pd(dppf)Cl₂ (0.1 eq), KOAc (3 eq), DMSO (251 mL), degassing 15 minutes N₂, reacting at 90° C. for 16 hours. Water was added to the reaction mix then extracted with EtOAc. The organic layer was washed with brine, dried over Na₂SO₄, concentrated to afford the boronate. 170 mg of the boronate was then reacted with 2-bromopyrimidine (0.5 eq), Pd(dppf)Cl₂ (0.1 eq), LiOH (3 eq), toluene (10 mL), EtOH (5 mL), H₂O (5 mL) degassed 15 minutes N₂, in a sealed tube at 110° C. for 16 hours. Water was added to the reaction and extracted with EtOAc. The organic layer was washed with brine, dried over Na₂SO₄ and concentrated to afford 2.

2 (400 mg), 1,4-dioxane (4 mL), 4.0 N HCl in 1,4-dioxane (4 mL) reacted at room temperature 16 hours. Volatiles were evaporated and purification by preparative HPLC gave 31 mg 1217.

### Example 56: Synthesis of Compound 1143 ((2R,3R,4R,5S)-1-{2-[3-({2-[(4-azido-2-nitrophenyl)amino]ethyl}amino)-5-chlorophenyl]ethyl}-2-(hydroxymethyl)piperidine-3,4,5-triol)

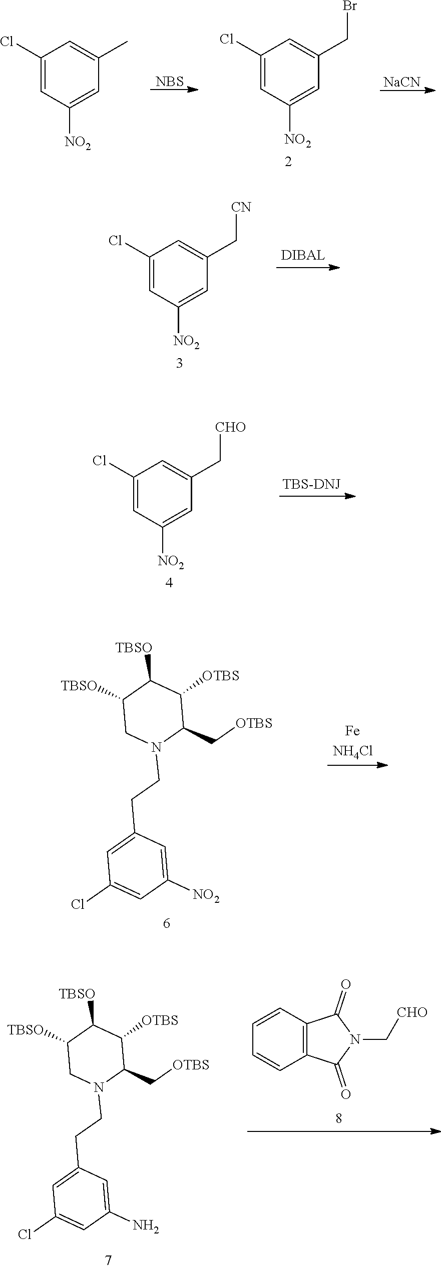

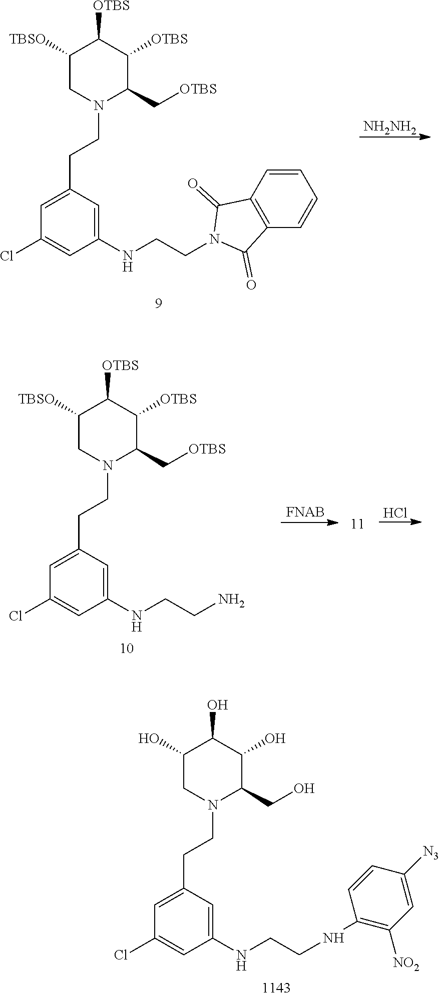 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCc1cc(Cl)cc(NCCN2C(=O)c3ccccc3C2=O)c1 ; "NH2NH2" ; CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCc1cc(Cl)cc(NCCN)c1 ; Cl ; [N-]=[N+]=Nc1ccc(NCCNc2cc(Cl)cc(CCN3C[C@H](O)[C@@H](O)[C@H](O)[C@H]3CO)c2)c([N+](=O)[O-])c1]

Mixed 3-chloro-5-nitro-toluene (10 g), ACN (200 mL), NBS (re-crystallized, 2.5 eq), AIBN (500 mg) at room temperature then at 80° C. for 24 hours. Diluted with water and extracted with EtOAc (2×50 mL), dried the organic layer over Na₂SO₄, filtered, concentrated. Two batches of this material were purified by COMBIFLASH [1% EtOAc-hexane] to afford 5 g of 2.

Mixed 2 (2.5 g), ethanol:water (3:1, 60 mL) at 0° C., added KCN (1.0 eq), refluxed 4 hours. Added water and extracted with EtOAc. The organic layer was washed with brine solution, dried over anhydrous Na₂SO₄, filtered, concentrated. Residue was purified on silica [5% EtOAc:hexane] to afford 700 mg of 3.

To 3 (1.4 g) in DCM (50 mL) at −78° C. added DIBAL-H (1.0 M in toluene, 1.1 eq) and stirred for 2 hours at −78° C. MeOH (20 eq) was added to the reaction dropwise at −78° C. then quenched with saturated Rochelle salt solution (50 mL) and stirred 30 minutes at room temperature. Aqueous layer was extracted with DCM. The DCM layer was washed with brine solution, dried over anhydrous Na₂SO₄, filtered, and concentrated to obtain 4.

4 (1.4 g), TBS-DNJ (1.2 eq), MeOH (60 mL) mixed at room temperature, added acetic acid (cat., 0.7 mL) and NaCNBH₃ (1.5 eq), stirred 16 hours at room temperature. Added water and extracted with EtOAc. Washed the organic layer with brine, dried over anhydrous Na₂SO₄, filtered, concentrated. The residue was purified on silica [5% EtOAc/hexane] to afford 600 mg of 6.

6 (600 mg), EtOH:water (3:1, 20 mL), iron powder (5.0 eq), ammonium chloride (5.0 eq) mixed at room temperature and increased to 80° C. After 2 hours the reaction was diluted with EtOAc and filtered through CELITE bed and concentrated. Water was added to the residue and extracted with EtOAc. The organic layer was washed with brine, dried over anhydrous Na₂SO₄, filtered, concentrated, and purified on silica [15% EtOAc/hexane] to afford 400 mg of 7.

2-(2-hydroxyethyl)isoindoline-1,3-dione (500 mg), EtOAc (20 mL), IBX (3.0 eq) mixed at room temperature then increased to 80° C. for 4 hours. Reaction was diluted with water and extracted with EtOAc. The organic layer was washed with brine solution, dried over anhydrous Na₂SO₄, filtered, concentrated, and washed with hexane to afford 400 mg of 8.

To 7 (400 mg) and 8 (1.2 eq) in MeOH (10 mL) at room temperature added acetic acid (cat.), NaCNBH₃ (1.5 eq) and stirred for 16 hours at room temperature. Water was added and extracted with EtOAc. The organic layer was washed with brine solution, dried over anhydrous Na₂SO₄, filtered, concentrated, and purified on silica [3% EtOAc/hexane] to afford 400 mg of 9.

To 9 (400 mg) in EtOH (10 mL) at room temperature added hydrazine hydrate (35%, 5.0 eq) and stirred at room temperature for 16 hours. EtOH was removed and water was added then extracted with EtOAc. The organic layer was dried over anhydrous Na₂SO₄, filtered, concentrated to obtain 10.

10 (300 mg), FNAB (1.0 eq), 1,4-dioxane (10 mL), Et₃N (3.0 eq) added at room temperature then increased to 80° C. After 16 hours, water was added and extracted with EtOAc. The organic layer was washed with brine, dried over anhydrous Na₂SO₄, filtered, concentrated. Residue was purified on silica [5% EtOAc/hexane] to afford 130 mg of 11.

11 (50 mg), 4.0 M HCl in 1,4-dioxane (0.5 mL), MeOH (5 mL) reacted at room temperature for 4 hours. The reaction was quenched with aqueous saturated NH₄CO₃ and solvents were removed to obtain 50 mg of material. Two combined batches were purified by preparative HPLC to afford 20.0 mg of 1143 as red solid.

### Example 57: Synthesis of Compound 1151 ((2R,3R,4R,5S)-1-(6-{[3-tert-butyl-5-(pyrimidin-2-yl)phenyl]amino}hexyl)-2-(hydroxymethyl)piperidine-3,4,5-triol)

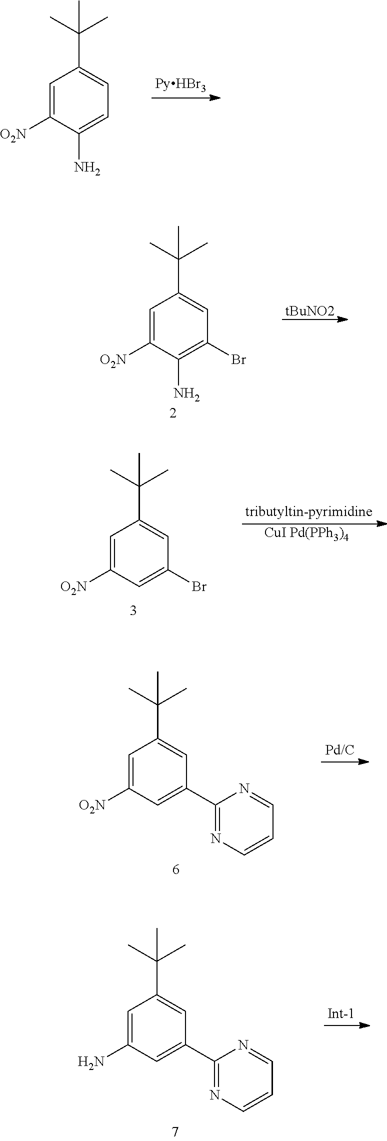

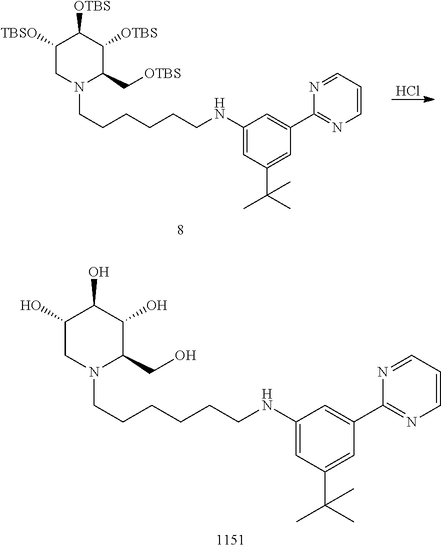 [SMILES: CC(C)(C)c1cc(NCCCCCCN2C[C@H](O[Si](C)(C)C(C)(C)C)[C@@H](O[Si](C)(C)C(C)(C)C)[C@H](O[Si](C)(C)C(C)(C)C)[C@H]2CO[Si](C)(C)C(C)(C)C)cc(-c2ncccn2)c1 ; Cl ; CC(C)(C)c1cc(NCCCCCCN2C[C@H](O)[C@@H](O)[C@H](O)[C@H]2CO)cc(-c2ncccn2)c1]

4-tert-Butyl-2-nitroaniline (2 g), AcOH (30 mL) were cooled to −10° C., added pyridinium hydrobromide perbromide (2 eq+0.5 eq) and stirred at room temperature for 8 hours. Quenched with 5 mL saturated hypo (sodium thiosulfate) solution and stirred for 30 minutes. Filtered the solid, washed with water then solid was dissolved in EtOAc. The organic layer was washed with water, brine, dried over anhydrous Na₂SO₄, filtered, concentrated to obtain 2 g of b as yellow solid.

To t-butyl nitrite (1.5 eq) in DMF (10 mL) at 50° C. added 2 (2 g) in DMF (15 mL), stirred for 1 hour at 50° C., added further 1.5 eq t-butyl nitrite and stirred for 1 hour at 50° C. Added ice cold water and extracted with EtOAc. Organic layer was washed with water, brine, dried over anhydrous Na₂SO₄, filtered, concentrated, and purified on silica [2% EtOAc:hexane] to afford 800 mg of 3.

3 (150 mg), DMF (2 mL), tributyltin pyrimidine (1.1 eq), KF (10.0 eq), CuI (cat.) was purged with argon, added Pd(PPh₃)₄ (0.1 eq) placed in sealed tube at 120° C. for 24 hours. Water was added and the reaction extracted with EtOAc. The organic layer was washed with water, brine, dried over anhydrous Na₂SO₄, filtered, concentrated, and purified on silica [10% EtOAc:hexane] and obtained 180 mg of 6.

6 (180 mg), MeOH (15 mL), 10% Pd/C (50% wet, 100 mg, 50% wet) was placed under H₂ atmosphere and stirred for 4 hours. Reaction was filtered through CELITE bed and washed with MeOH. The combined organic layer was concentrated to obtain 120 mg of 7.

7 (105 mg), MeOH (10 mL), Int-1 (400 mg, 1.2 eq), AcOH (cat.) mixed at 0° C., stirred 5 minutes, NaCNBH₃ (1.5 eq) added, warmed to room temperature. After 16 hours the solvent was removed, water was added and the residue extracted with EtOAc. The organic layer was washed with brine, dried over anhydrous Na₂SO₄, filtered, concentrated, and purified on silica [15% EtOAc:hexane] to yield 170 mg of 8.

8 (170 mg), MeOH (15 mL), 4.0 M HCl in 1,4-dioxane (5 mL) mixed at 0° C., warmed to room temperature, stirred for 16 hours. The solvent was removed. Purification of combined batches by preparative HPLC afforded 30 mg of 1151.

### Example 58: Synthesis of Compounds 1157 ((2R,3R,4R,5S)-2-(hydroxymethyl)-1-{2-[4-(2-{[3-methoxy-5-(pyridazin-3-yl)phenyl]amino}ethyl)phenyl]ethyl}piperidine-3,4,5-triol), 1156 ((2R,3R,4R,5S)-2-(hydroxymethyl)-1-{2-[4-(2-{[3-methoxy-5-(pyrimidin-2-yl)phenyl]amino}ethyl)phenyl]ethyl}piperidine-3,4,5-triol), 1155 ((2R,3R,4R,5S)-1-{2-[4-(2-{[3-(5,6-dihydro-1,4-dioxin-2-yl)-5-methoxyphenyl]amino}ethyl)phenyl]ethyl}-2-(hydroxymethyl)piperidine-3,4,5-triol), 1154 ((2R,3R,4R,5S)-1-{2-[4-(2-{[3-(3,6-dihydro-2H-pyran-4-yl)-5-methoxyphenyl]amino}ethyl)phenyl]ethyl}-2-(hydroxymethyl)piperidine-3,4,5-triol), and 1153 ((2R,3R,4R,5S)-1-{2-[4-(2-{[3-(furan-2-yl)-5-methoxy-phenyl]amino}ethyl)phenyl]ethyl}-2-(hydroxymethyl)piperidine-3,4,5-triol)

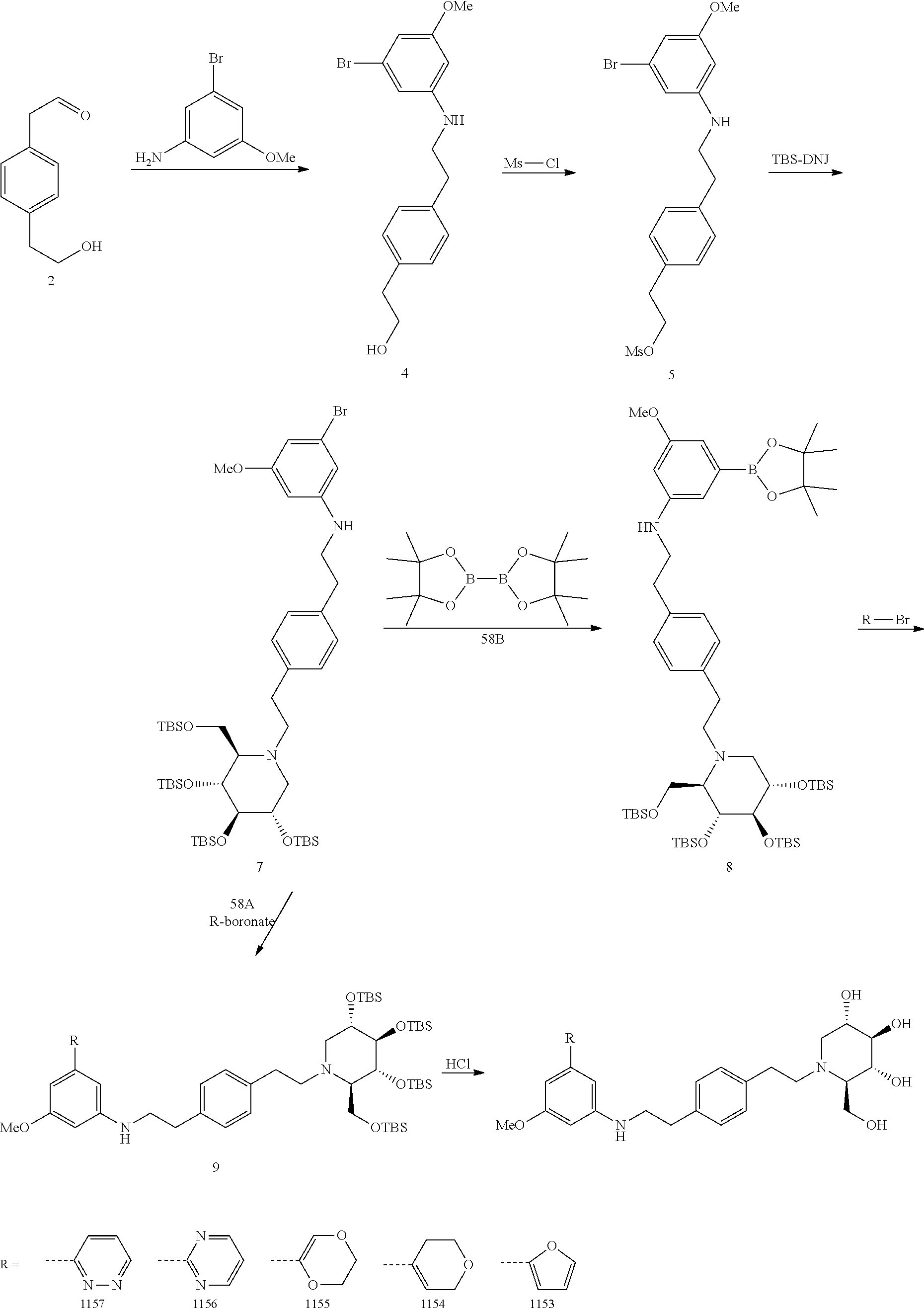

2 (7.0 g, Example 50), DCM/MeOH (1:1, 100 mL), 3-bromo-5-methoxy-aniline (1 eq), AcOH (0.1 mL) mixed at 0° C., NaCNBH₃ (1.5 eq) added, raised to room temperature. After 16 hours solvent was removed, the residue quenched with ice-cold water, and extracted with EtOAc (2×15 mL). The organic layer was dried was over anhydrous Na₂SO₄ and concentrated. Residue was purified on silica [20% EtOAc-hexane] to afford 9 g of 4.

To 4 (9 g) in DCM (150 mL) at 0° C. added TEA (3.0 eq), Ms-Cl (1.1 eq), stirred at 0° C. for 30 minutes. Solvent was removed and residue quenched with ice-cold water and extracted with EtOAc (2×15 mL). The organic layer was dried was over anhydrous Na₂SO₄ and concentrated. Residue was purified on silica [20% EtOAc in hexane to afford 7 g of 5.

5 (7.0 g), ACN (70 mL), K₂CO₃ (3.0 eq), TBS-DNJ (1.0 eq) reacted at 80° C. for 24 hours. The solvent was removed, residue quenched with ice-cold water and extracted with EtOAc (2×15 mL). The organic layer was dried was over anhydrous Na₂SO₄ then concentrated. Residue was purified on silica [60-120 mesh, 3% EtOAc:hexane] to afford 6 g of 7.

For scheme 58B, in one example, 7 (2 g), 1,4-dioxane (50 mL), bis(pinacolato) diboron (1.5 eq), KOAc (3 eq), were purged with N₂ for 30 minutes, added Pd(dppf)Cl₂ (0.1 eq), heated 16 hours at 90° C. Reaction was diluted with water, extracted with EtOAc (2×30 mL). The organic layer was dried over Na₂SO₄, filtered, concentrated to give 1.5 g of 8.

Preparation of 1153 (58A) (2R,3R,4R,5S)-1-{2-[4-(2-{[3-(furan-2-yl)-5-methoxy-phenyl]amino}ethyl)phenyl]ethyl}-2-(hydroxymethyl)piperidine-3,4,5-triol: 7 (500 mg), toluene:EtOH:H₂O (1:1:1, 30 mL), 2-furyl boronate (1.5 eq), degassed 15 minutes N₂, added Pd(dppf)Cl₂ (0.05 eq), Na₂CO₃ (3 eq), reacted at 80° C. for 16 hours. The reaction was quenched with ice-cold water and extracted with EtOAc (2×10 mL). The organic layer was dried was over anhydrous Na₂SO₄ and concentrated. Residue was purified on silica [3% EtOAc:hexane] to yield 9 (300 mg).

To 9 (300 mg) in MeOH (8 mL) at 0° C. added 4.0 M HCl in 1,4-dioxane (2.0 mL), mixed at room temperature 4 hours. The reaction was concentrated and preparative HPLC purification gave 40 mg of 1153.

Preparation of 1154 (58A) (2R,3R,4R,5S)-1-{2-[4-(2-{[3-(3,6-dihydro-2H-pyran-4-yl)-5-methoxyphenyl]amino}ethyl)phenyl]ethyl}-2-(hydroxymethyl)piperidine-3,4,5-triol: Mixed 7 (500 mg), toluene:EtOH:H₂O (1:1:1, 30 mL), 2-(3,6-dihydro-2H-pyran-4-yl)-boronate (1.5 eq), degassed 15 minutes with N₂, added Pd₂(dppf)Cl₂ (0.05 eq), Na₂CO₃ (3 eq), heated at 80° C. 16 hours. Quenched with ice-cold water, extracted with EtOAc (2×10 mL), dried the organic layer over anhydrous Na₂SO₄ and concentrated. Residue was purified on silica [15% EtOAc in hexane to afford 180 mg of 9.

To 9 (180 mg) in MeOH (5 mL) at 0° C. added 4.0 M HCl in 1,4-dioxane (1.5 mL), reacted at room temperature for 4 hours. The reaction mass was concentrated. Residue was purified by preparative HPLC to afford 17 mg of 1154 as colorless thick syrup.

Preparation of 1155 (58A) (2R,3R,4R,5S)-1-{2-[4-(2-{[3-(5,6-dihydro-1,4-dioxin-2-yl)-5-methoxyphenyl]amino}ethyl)phenyl]ethyl}-2-(hydroxymethyl)piperidine-3,4,5-triol: 7 (250 mg), 1,4-dixoxane:H₂O (3:1, 9 mL), 2-(5,6-dihydro-1,4-dioxin-2-yl)-boronate (1.5 eq), degassed 15 minutes N₂, added Pd(dppf)Cl₂ (0.1 eq), Na₂CO₃ (3 eq), heated at 100° C. for 8 hours. The reaction was quenched with ice-cold water and extracted with EtOAc (2×20 mL). The organic layer was dried was over anhydrous Na₂SO₄ then concentrated. Residue was purified on silica [10% EtOAc:hexane] to afford 210 mg of 9.

To 9 (200 mg) in MeOH (4 mL) at 0° C. added 4.0 MHCl in dioxane (2.0 mL) at 0° C. then to room temperature for 8 hours. The reaction was concentrated. Purification by preparative HPLC yielded 13.0 mg of 1155.

Preparation of 1156 (58B) (2R,3R,4R,5S)-2-(hydroxymethyl)-1-{2-[4-(2-{[3-methoxy-5-(pyrimidin-2-yl)phenyl]amino}ethyl)phenyl]ethyl}piperidine-3,4,5-triol: 8 (500 mg), toluene:ethanol:water (1:1:1) (30 mL), 2-bromopyrimidine (1.5 eq), Na₂CO₃ (3 eq), 30 minutes N₂ purged, added Pd(dppf)Cl₂ (0.1 eq), mixed at 90° C. for 16 hours, diluted reaction with water, extracted with EtOAc (2×10 mL), organic layer dried over Na₂SO₄, filtered, concentrated, purified on silica [5% EtOAc:hexane] to give 150 mg of 9.

9 (170 mg) and 4M HCl in 1,4-dioxane (2.0 mL) in MeOH (3.0 mL) at 0° C. were heated to room temperature for 16 hours. Reaction was concentrated then purified by preparative HPLC to afford 35 mg of 1156.

Preparation of 1157 (58B) (2R,3R,4R,5S)-2-(hydroxymethyl)-1-{2-[4-(2-{[3-methoxy-5-(pyridazin-3-yl)phenyl]amino}ethyl)phenyl]ethyl}piperidine-3,4,5-triol: Mixed 8 (700 mg), toluene:EtOH:H₂O (1:1:1, 40 mL), 3-bromopyridazine (1.5 eq), degassed 15 minutes N₂, added Pd(dppf)Cl₂ (0.1 eq) Na₂CO₃ (3 eq), heated at 90° C. for 16 hours. Reaction was diluted with water and extracted with EtOAc (2×20 mL). Organic layer was dried over Na₂SO₄, filtered, concentrated. Material was purified on silica [5% EtOAC:hexane] to afford 270 mg of 9.

9 (300 mg), 4.0 M HCl in 1,4-dioxane (3.0 mL), MeOH (5.0 mL), mixed at 0° C. then to room temperature for 16 hours. Reaction was concentrated. Material was purified by preparative HPLC to afford 50 mg of 1157.

### Example 59: Synthesis of Compounds 1163 ((2R,3R,4R,5S)-1-{2-[4-(2-{[3-chloro-5-(pyridazin-3-yl)phenyl]amino}ethyl)phenyl]ethyl}-2-(hydroxymethyl)piperidine-3,4,5-triol), 1162 ((2R,3R,4R,5S)-1-{2-[4-(2-{[3-chloro-5-(pyrimidin-2-yl)phenyl]amino}ethyl)phenyl]ethyl}-2-(hydroxymethyl)piperidine-3,4,5-triol), 1161 ((2R,3R,4R,5S)-1-{2-[4-(2-{[3-chloro-5-(5,6-dihydro-1, 4-dioxin-2-yl)phenyl]amino}ethyl)phenyl]ethyl}-2-(hydroxymethyl)piperidine-3,4,5-triol), 1160 ((2R,3R,4R,5S)-1-{2-[4-(2-{[3-chloro-5-(3,6-dihydro-2H-pyran-4-yl)phenyl]amino}ethyl)phenyl]ethyl}-2-(hydroxymethyl)piperidine-3,4,5-triol), and 1159 ((2R,3R,4R,5S)-1-{2-[4-(2-{[3-chloro-5-(furan-2-yl)phenyl]amino}ethyl)phenyl]ethyl}-2-(hydroxymethyl)piperidine-3,4,5-triol)

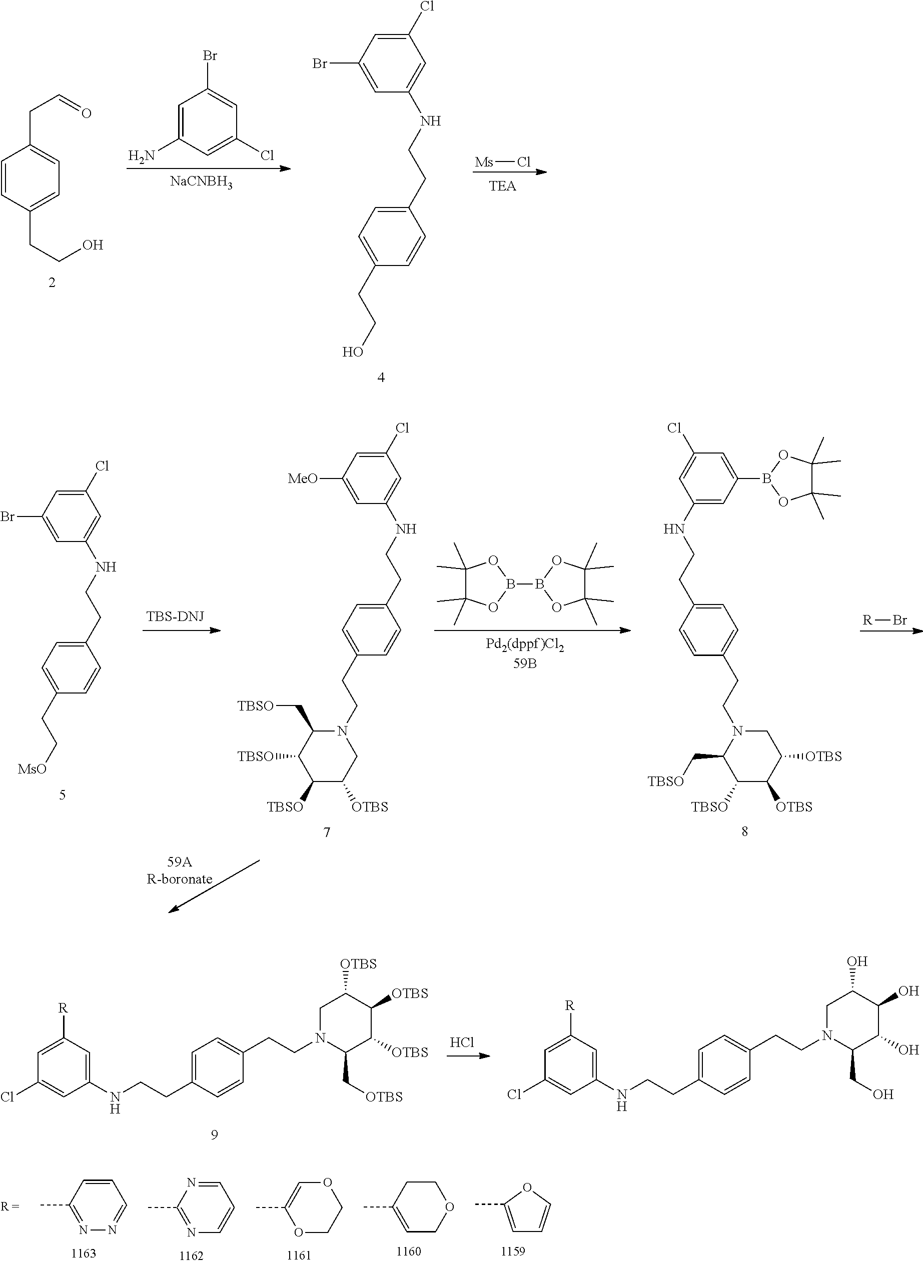

2 (3 g, Example 50), DCM/MeOH (1:1, 100 mL), 3-chloro-5-bromoaniline (1 eq), AcOH (0.01 mL) at 0° C., added NaCNBH₃ (1.5 eq), increase to room temperature, react 16 hours. Solvent was removed and residue was quenched with ice-cold water and extracted with EtOAc (2×100 mL). The organic layer dried was over anhydrous Na₂SO₄ and concentrated. Material was purified on silica [5% EtOAc:hexane] to afford 1.8 g of 4.

4 (1.8 g), DCM, TEA (3 eq), Ms-Cl (1.5 eq) reacted at 0° C. for 30 minutes. The reaction was quenched with ice-cold water and extracted with DCM (2×100 mL). The organic layer dried was over anhydrous Na₂SO₄, concentrated, and purified on silica [15% EtOAc:hexane] to afford 1.8 g of 5.

5 (1.8 g), ACN (5 mL), K₂CO₃ (3 eq), TBS-DNJ (1 eq) heated at 80° C. for 48 hours. The reaction was cooled to room temperature, quenched with ice-cold water, and extracted with EtOAc (2×50 mL). The organic layer was dried was over anhydrous Na₂SO₄ and concentrated. Material was purified on silica [4% EtOAc:hexane] to afford 1.2 g of 7.

For scheme B, in one example, 7 (250 mg), 1,4-dioxane (10 mL) purged with N₂ 30 minutes, added bis(pinacolato) diboron (1.5 eq), Pd(dppf)Cl₂ (0.1 eq), KOAc (3 eq), heated to 90° C. for 16 hours. The reaction was cooled to room temperature, distilled. Residue was dissolved in water and extracted with EtOAc (2×10 mL). The organic layer was dried over Na₂SO₄, filtered, concentrated to afford 250 mg of 8.

Preparation of 1159 (59A) (2R,3R,4R,5S)-1-{2-[4-(2-{[3-chloro-5-(furan-2-yl)phenyl]amino}ethyl)phenyl]ethyl}-2-(hydroxymethyl)piperidine-3,4,5-triol: 7 (300 mg), toluene:EtOH:H₂O (1:1:1, 21 mL), 2-furyl boronate (1.5 eq), degassed 15 minutes N₂, added Pd(dppf)Cl₂ (0.05 eq), Na₂CO₃ (3 eq) reacted at 80° C. for 16 hours. The reaction was cooled to room temperature, diluted with water, and extracted with EtOAc (2×15 mL). The organic layers were dried over Na₂SO₄, filtered, concentrated. Combined batches were purified on silica [5% EtOAc in hexane] to afford 300 mg of 9.

9 (300 mg), MeOH/DCM (1:1, 6 mL), 4M HCl in dioxane (3 mL) reacted 8 hours at room temperature. The reaction was concentrated. Preparative HPLC purification gave 80 mg of 1159.

Preparation of 1160 (59A) (2R,3R,4R,5S)-1-{2-[4-(2-{[3-chloro-5-(3,6-dihydro-2H-pyran-4-yl)phenyl]amino}ethyl)phenyl]ethyl}-2-(hydroxymethyl)piperidine-3,4,5-triol: 7 (300 mg), toluene:EtOH:H₂O (1:1:1, 21 mL), 2-(3,6-dihydro-2H-pyran-4-yl)-boronate (1.5 eq), degassed 15 minutes N₂, added Pd(dppf)Cl₂ (0.05 eq), Na₂CO₃ (3 eq) reacted at 80° C. for 16 hours. The reaction was cooled to room temperature, diluted with water and extracted with EtOAc (2×15 mL). The organic layers were dried over Na₂SO₄, filtered, concentrated. Combined batches were purified on silica [5% EtOAc in hexane] to afford 300 mg of 9.

9 (300 mg), MeOH/DCM (1:1, 6 mL), 4.0 M HCl in dioxane (3 mL) reacted 8 hours at room temperature. Reaction was concentrated. Preparative HPLC purification afforded 80 mg of 1160.

Preparation of 1161 (59A) (2R,3R,4R,5S)-1-{2-[4-(2-{[3-chloro-5-(5,6-dihydro-1,4-dioxin-2-yl)phenyl]amino}ethyl)phenyl]ethyl}-2-(hydroxymethyl)piperidine-3,4,5-triol: 7 (250 mg), dioxane:water (3:1, 13 mL), 2-(5,6-dihydro-1,4-dioxin-2-yl)-boronate (1.5 eq), degassed 15 minutes N₂, added Pd(dppf)Cl₂ (0.06 eq), Cs₂CO₃ (3 eq), reacted in sealed tube at 100° C. for 9 hours. The reaction was cooled to room temperature, diluted with water and extracted with EtOAc (2×20 mL). The organic layer was dried over Na₂SO₄, filtered, concentrated. Residue was purified on silica [10% EtOAc in hexane] to afford 150 mg of 9.

9 (150 mg), MeOH (2 mL), DCM (2 mL), mixed at 0° C., added 4M HCl in dioxane (1.5 mL), increased to room temperature for 8 hours. The reaction was distilled. Preparative HPLC purification yielded 17 mg of 1161.

Preparation of 1162 (59B) (2R,3R,4R,5S)-1-{2-[4-(2-{[3-chloro-5-(pyrimidin-2-yl)phenyl]amino}ethyl)phenyl]ethyl}-2-(hydroxymethyl)piperidine-3,4,5-triol: Mixed 8 (250 mg), 2-bromopyrimidine (1.3 eq), toluene:EtOH:water (1:1:1, 16 mL), Na₂CO₃ (3 eq), degassed 15 minutes N₂, added Pd(dppf)Cl₂ (0.06 eq) reacted 16 hours at 80° C. The reaction was cooled to room temperature, diluted with water and extracted with EtOAc (2×15 mL). The organic layer was dried over Na₂SO₄, filtered, concentrated. Combined batches were purified on silica [10% EtOAc in hexane] to afford 280 mg of 9.

9 (280 mg), MeOH (2 mL), DCM (2 mL) mixed at 0° C., added 4 MHCl in dioxane (2.5 mL), reacted at room temperature for 8 hours. The reaction was concentrated which after preparative HPLC purification afforded 20 mg of 1162.

Preparation of 1163 (59B) (2R,3R,4R,5S)-1-{2-[4-(2-{[3-chloro-5-(pyridazin-3-yl)phenyl]amino}ethyl)phenyl]ethyl}-2-(hydroxymethyl)piperidine-3,4,5-triol: Mixed 8 (200 mg), 3-bromopyridazine (1.3 eq), toluene:EtOH:water (1:1:1, 24 mL), Na₂CO₃ (3 eq), degassed 15 minutes N₂, added Pd(dppf)Cl₂ (0.06 eq), reacted at 80° C., 16 hours. The reaction was cooled to room temperature, diluted with water and extracted with EtOAc (2×20 mL). The organic layer was dried over Na₂SO₄, filtered, concentrated. Combined batches were purified on silica [10% EtOAc in hexane] to afford 125 mg of 9.

9 (125 mg), MeOH (1 mL), DCM (1 mL), mixed at 0° C., added 4.0 MHCl in dioxane (1.5 mL), reacted at room temperature 8 hours. The reaction was concentrated. After preparative HPLC purification 24 mg of 1163 was obtained.

### Example 60: Synthesis of Compound 1166 ((2R,3R,4R,5S)-1-[(4′-{[(4-azido-2-nitrophenyl)amino]methyl}-[1,1′-biphenyl]-4-yl)methyl]-2-(hydroxymethyl)piperidine-3,4,5-triol)

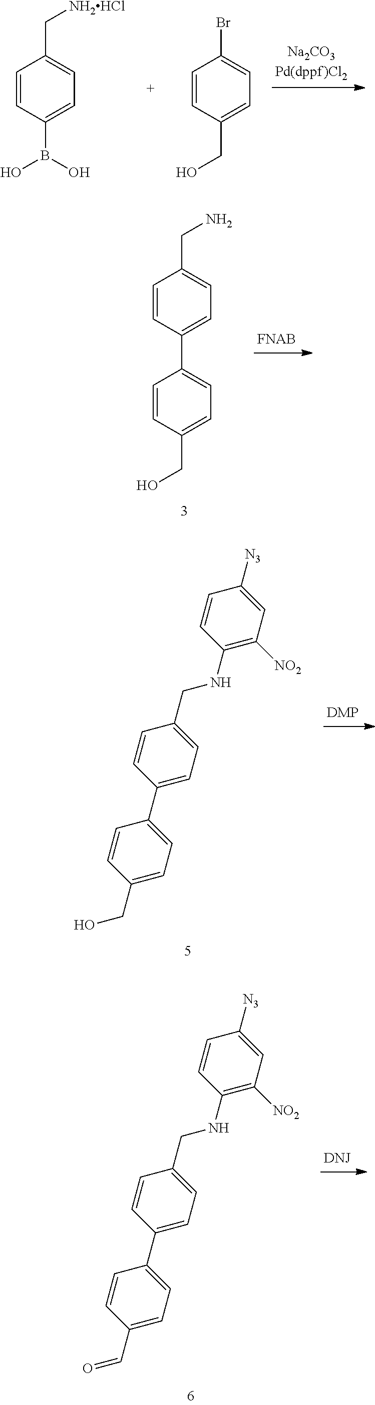

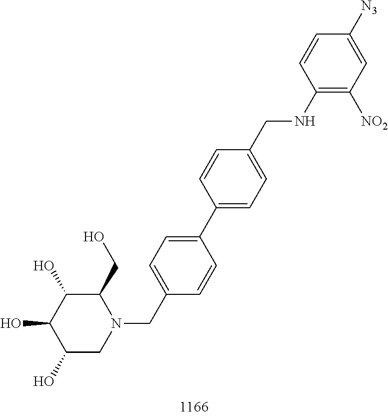 [SMILES: [N-]=[N+]=Nc1ccc(NCc2ccc(-c3ccc(CN4C[C@H](O)[C@@H](O)[C@H](O)[C@H]4CO)cc3)cc2)c([N+](=O)[O-])c1]

Purged 2-(4-bromophenyl)ethanol (500 mg), (4-(2-aminoethyl)phenyl)boronic acid (1.2 eq) EtOH:toluene:H₂O (15 mL, 1:1:1), Na₂CO₃ (5.0 eq) with N₂, added Pd(dppf)Cl₂ (0.1 eq), reacted 3 hours at 70° C. The reaction was concentrated, residue was diluted with water and extracted with EtOAc (2×10 mL). Combined organic layer was dried over Na₂SO₄, filtered, concentrated, and purified by acid base treatment to give 350 mg of 3.

3 (350 mg), 1,4-dioxane (10 mL), Et₃N (3.0 eq) FNAB (1.1 eq) reacted 60° C., 4 hours. The reaction was concentrated. Material was purified on silica [20-50% EtOAc in hexane] to afford 280 mg of 5.

5 (280 mg) in DCM (10 mL) at 0° C., added DMP (1.2 eq), increase to room temperature for 2 hours. Reaction was quenched with aq. NaHCO₃ and extracted with EtOAc which was washed with brine, dried over Na₂SO₄ and evaporated to provide 6.

6 (260 mg), MeOH (8 mL), DNJ (1.1 eq), AcOH (0.1 mL) mixed at 0° C., NaCNBH₃ (1.2 eq), increased to room temperature for 12 hours. Reaction was concentrated to afford residue which was washed with Et₂O. 1166 (38 mg) was isolated after preparative HPLC purification.

### Example 61: Synthesis of Compound 1169 ((2R,3R,4R,5S)-1-[(3-{[(4-azido-2-nitrophenyl)amino]methyl}phenyl)methyl]-2-(hydroxymethyl)piperidine-3,4,5-triol)

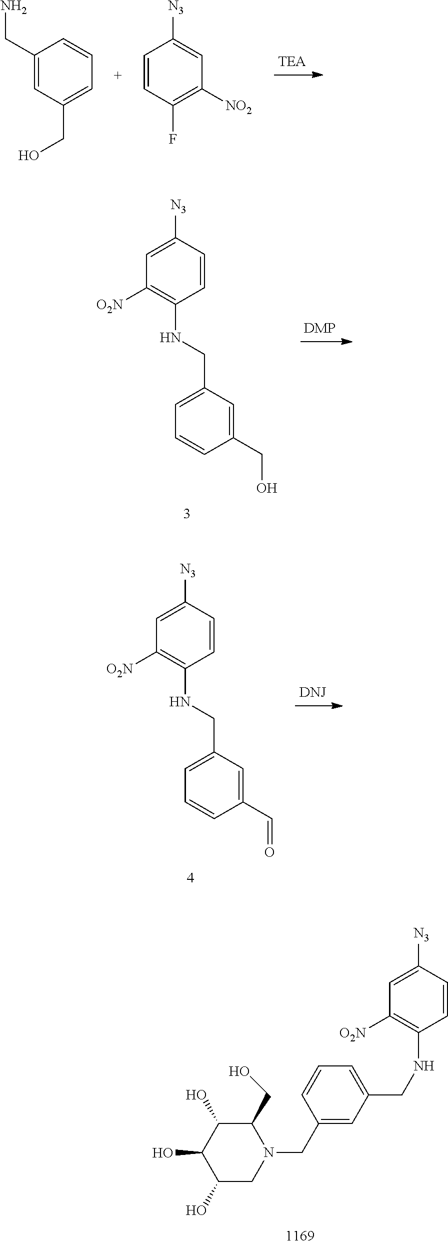 [SMILES: NCc1cccc(CO)c1 ; [N-]=[N+]=Nc1ccc(F)c([N+](=O)[O-])c1 ; [N-]=[N+]=Nc1ccc(NCc2cccc(CO)c2)c([N+](=O)[O-])c1 ; [N-]=[N+]=Nc1ccc(NCc2cccc(C=O)c2)c([N+](=O)[O-])c1 ; [N-]=[N+]=Nc1ccc(NCc2cccc(CN3C[C@H](O)[C@@H](O)[C@H](O)[C@H]3CO)c2)c([N+](=O)[O-])c1]

3-(Aminomethyl)phenyl)methanol (500 mg), 1,4-dioxane (10 mL), FNAB (1 eq), TEA (3.0 eq) heated at 100° C. for 4 hours. Solvent was removed and residue was diluted with water and extracted with EtOAc (2×50 mL). The organic layer was dried over anhydrous Na₂SO₄, concentrated, and purified on silica [10% EtOAc:hexane] to afford 600 mg of 3.

3 (500 mg), DCM (15 mL), DMP (1.5 eq) was reacted at 0° C. for 2 hours. The reaction was quenched with aq. NaHCO₃ and extracted with DCM (2×50 mL). The organic layer was filtered on CELITE, washed with water then brine solution, dried over anhydrous Na₂SO₄ and concentrated to afford 4 (500 mg).

4 (500 mg), MeOH (2 mL), DNJ (1.2 eq), AcOH (0.05 mL) mixed at 0° C., NaCNBH₃ (1.5 eq) added then increased to room temperature. After 16 hours the reaction mass was concentrated. Preparative HPLC purification gave 150 mg of 1169.

### Example 62: Synthesis of Compound 1170 ((2R,3R,4R,5S)-1-[(4-{[(4-azido-2-nitrophenyl)amino]methyl}phenyl)methyl]-2-(hydroxymethyl)piperidine-3,4,5-triol)

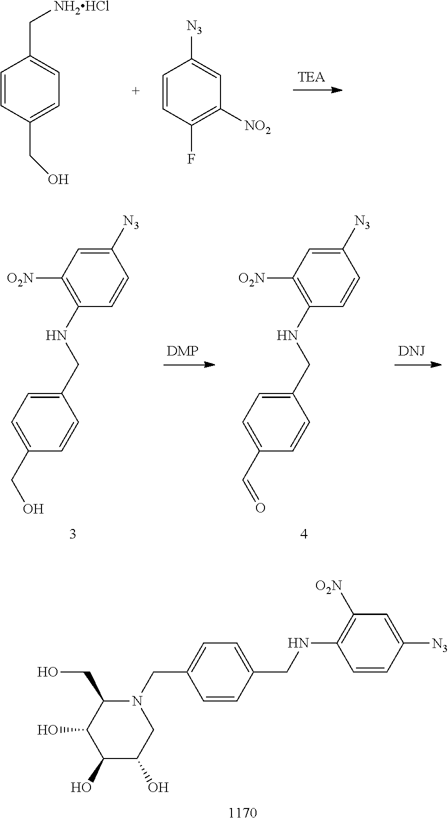

4-(Aminomethyl)phenyl)methanol (500 mg), 1,4-dioxane (10 mL), FNAB (1 eq), TEA (5.0 eq) reacted 100° C. for 4 hours. Solvent was removed and residue diluted with water and extracted with EtOAc. The organic layer was dried over anhydrous Na₂SO₄ and concentrated, and purified on silica [10% EtOAc:hexane] to afford 350 mg of 3.

3 (250 mg), DCM (15 mL), DMP (1.5 eq) reacted at 0° C. for 2 hours. The reaction was quenched with aq. NaHCO₃ and extracted with DCM. The organic layer was washed with water, brine, dried over anhydrous Na₂SO₄ and concentrated to afford 4 (200 mg).

4 (200 mg), MeOH (10 mL), DNJ (1.2 eq), AcOH (0.05 mL) mixed at 0° C., NaCNBH₃ (1.5 eq) added and temperature increased to room temperature. After 24 hours the reaction was concentrated and combined batches purified by preparative HPLC to afford 30 mg of 1170.

### Example 63: Synthesis of Compound 1171 ((2R,3R,4R,5S)-1-[2-(3-{2-[(4-azido-2-nitrophenyl)amino]ethyl}-4-cyclohexylphenyl)ethyl]-2-(hydroxymethyl)piperidine-3,4,5-triol)

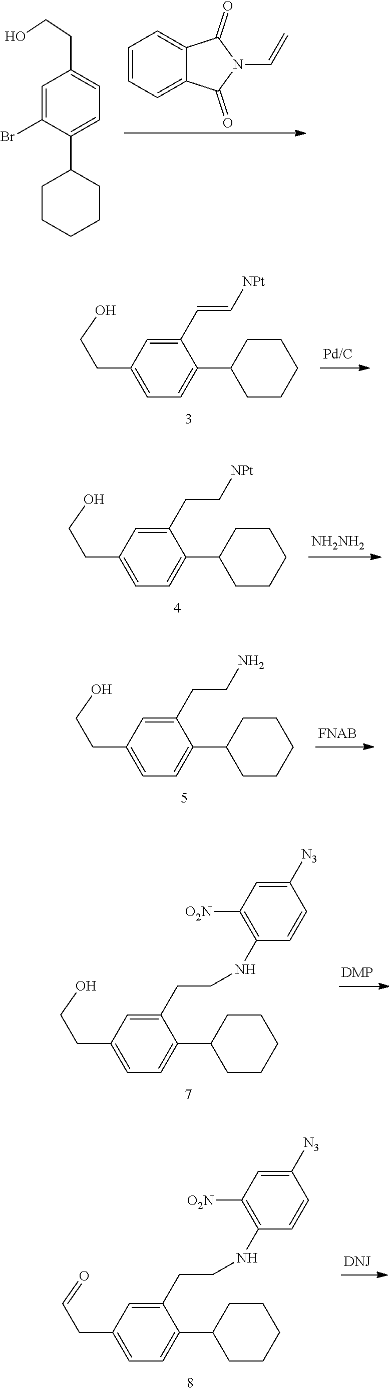

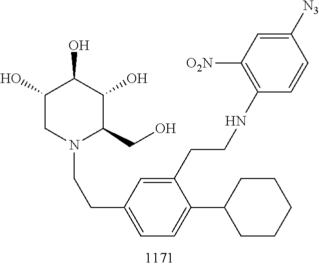 [SMILES: [N-]=[N+]=Nc1ccc(NCCc2cc(CCN3C[C@H](O)[C@@H](O)[C@H](O)[C@H]3CO)ccc2C2CCCCC2)c([N+](=O)[O-])c1]

2-(3-bromo-4-cyclohexylphenyl)ethan-1-ol (350 mg), ACN (10 mL), 2-vinylisoindoline-1,3-dione (1.5 eq), Et₃N (4.0 eq), tri(o-tolyl)phosphine (0.2 eq) was degassed 10 minutes with N₂, Pd(OAc)₂ (0.1 eq) added then increased to 100° C. in a sealed tube. After 48 hours reaction mass was diluted with water and extracted with EtOAc (2×25 mL). The combined organic layers were washed with brine solution, dried over anhydrous Na₂SO₄, filtered, concentrated, and purified on silica [15% EtOAc in hexane] to afford 210 mg of 3.

3 (210 mg), MeOH:THF (1:1, 15 mL), 10% Pd/C (50% wet, 120 mg) reacted under H₂ pressure (balloon) 48 h at room temperature. Filtered through CELITE, washed with EtOAc, concentrated, and purified on silica [20% EtOAc in hexane] to afford 120 mg of 4.

4 (120 mg), EtOH (7 mL), 35% N₂H₄ (5.0 eq) reacted at room temperature. After 16 hours volatiles were removed, residue was dissolved in water and extracted with EtOAc. The organic layer was dried over anhydrous Na₂SO₄ then concentrated to afford 100 mg of 5.

5 (100 mg), 1,4-dioxane (10 mL), Et₃N (3.0 eq), FNAB (1.0 eq), reacted at 100° C. After 16 hours volatiles were removed. The residue was dissolved in water and extracted with EtOAc. The organic layer was dried over anhydrous Na₂SO₄, concentrated, and purified on silica [30% EtOAc-hexane] to afford 50 mg of 7.

7 (50 mg), DCM (3 mL) mixed at 0° C., DMP (2.5 eq) added maintaining at 0° C., then warmed to room temperature. After 1.5 hours reaction diluted with water and extracted with DCM. The organic layer was dried over anhydrous Na₂SO₄ then concentrated to afford 50 mg of 8.

Reacted 8 (50 mg), MeOH (5 mL), DNJ (1.2 eq), AcOH (cat.), NaCNBH₃ (1.5 eq) at room temperature. After 16 hours solvent was removed. Residue was purified by preparative HPLC. Product was triturated with n-hexane and after lyophilization 7 mg of 1171 was obtained.

### Example 64: Synthesis of Compound 1173 ((2R,3R,4R,5S)-1-[2-(8-{2-[(4-azido-2-nitrophenyl)amino]ethyl}-2,3-dihydro-1,4-benzodioxin-5-yl)ethyl]-2-(hydroxymethyl)piperidine-3,4,5-triol)

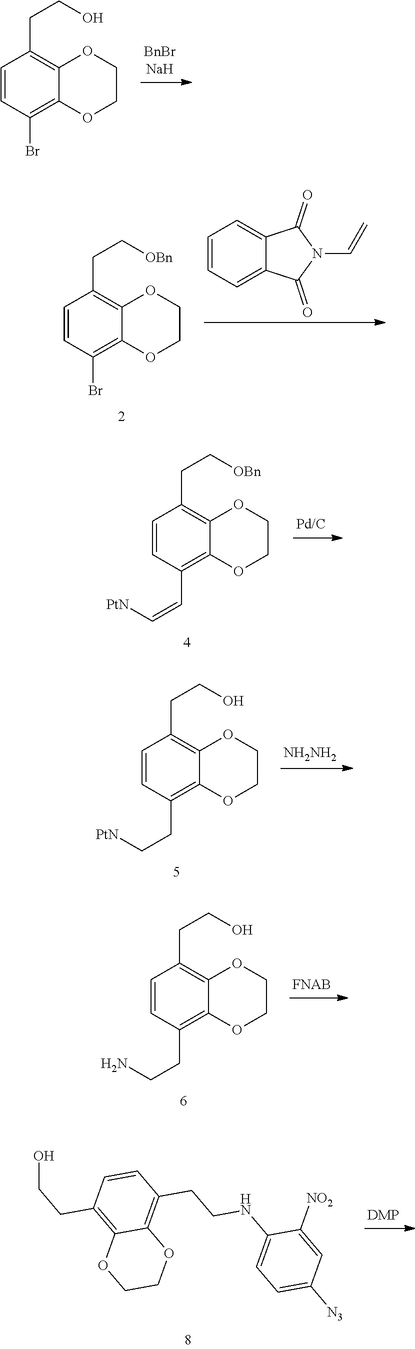

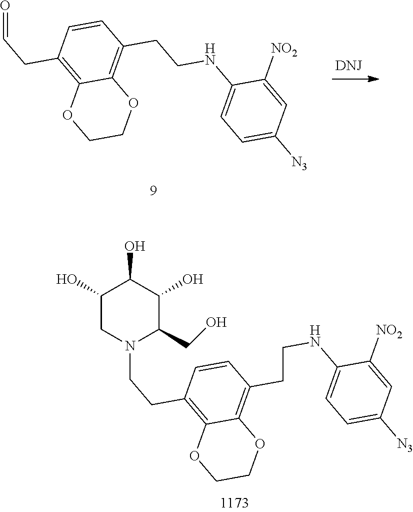 [SMILES: [N-]=[N+]=Nc1ccc(NCCc2ccc(CC=O)c3c2OCCO3)c([N+](=O)[O-])c1 ; "DNJ" ; [N-]=[N+]=Nc1ccc(NCCc2ccc(CCN3C[C@H](O)[C@@H](O)[C@H](O)[C@H]3CO)c3c2OCCO3)c([N+](=O)[O-])c1]

2-(8-bromo-2,3-dihydrobenzo[b][1,4]dioxin-5-yl)ethan-1-ol (2.0 g), THE (80 mL) at 0° C., NaH (1.2 eq) added and mixed 30 minutes, BnBr (1.1 eq) added and increased to room temperature. After 16 hours reaction was diluted with water and extracted with EtOAc. The organic layer was dried over anhydrous Na₂SO₄ and concentrated. Material was purified on silica [10% EtOAc in hexane] to afford 2.0 g of 2 as colorless thick syrup.

2 (2.0 g), hexafluoro-2-propanol (50 mL), 2-vinylisoindoline-1,3-dione (1.2 eq), acetyl glycine (0.2 eq), Ag₂CO₃ (2.0 eq) degassed 10 minutes with N₂, Pd(OAc)₂ (0.1 eq) added and increased to 80° C. After 16 hours solvent was removed. Material was purified on silica [10% EtOAc in hexane] to afford 700 mg of 4 as colorless thick syrup.

4 (700 mg), MeOH (10 mL), THE (10 mL), 10% Pd/C (50 mg) reacted under H₂ pressure (balloon) at room temperature. After 6 hours the reaction mass was filtered through CELITE, filtrate was concentrated. Material was purified on silica [50% EtOAc-hexane] to afford 400 mg of 5 as colorless thick syrup.

5 (350 mg), EtOH (10 mL), 35% N₂H₄ (5.0 eq) reacted at room temperature. After 16 hours removed the solvent, diluted with water and extracted with EtOAc. The organic layer was dried over anhydrous Na₂SO₄ concentrated to afford 220 mg of 6.

6 (220 mg), 1,4-dioxane (15 mL), TEA (3.0 eq), FNAB (1.0 eq) maintained at 100° C. After 16 hours the solvent was removed, residue diluted with water and extracted with EtOAc. The organic layer was dried over anhydrous Na₂SO₄ concentrated. Material was purified on silica [30% EtOAc in hexane] to afford 110 mg of 8.

To 8 (80 mg) in DCM (10 mL) at 0° C., DMP (2.5 eq) added in portions, increased to room temperature. After 1.5 hours reaction mass diluted with water and extracted with DCM. The organic layer was dried over anhydrous Na₂SO₄ concentrated to afford 80 mg of 9.

9 (80 mg), MeOH (8 mL), DNJ (1.0 eq), AcOH (cat.), NaCNBH₃ (1.5 eq) maintained at room temperature. After 16 hours solvent was removed. Combined batches were purified by preparative HPLC to afford 13 mg of 1173.

### Example 65: Synthesis of Compound 1174 ((2R,3R,4R,5S)-1-[2-(5-{[(4-azido-2-nitrophenyl)amino]methyl}thiophen-3-yl)ethyl]-2-(hydroxymethyl)piperidine-3,4,5-triol)

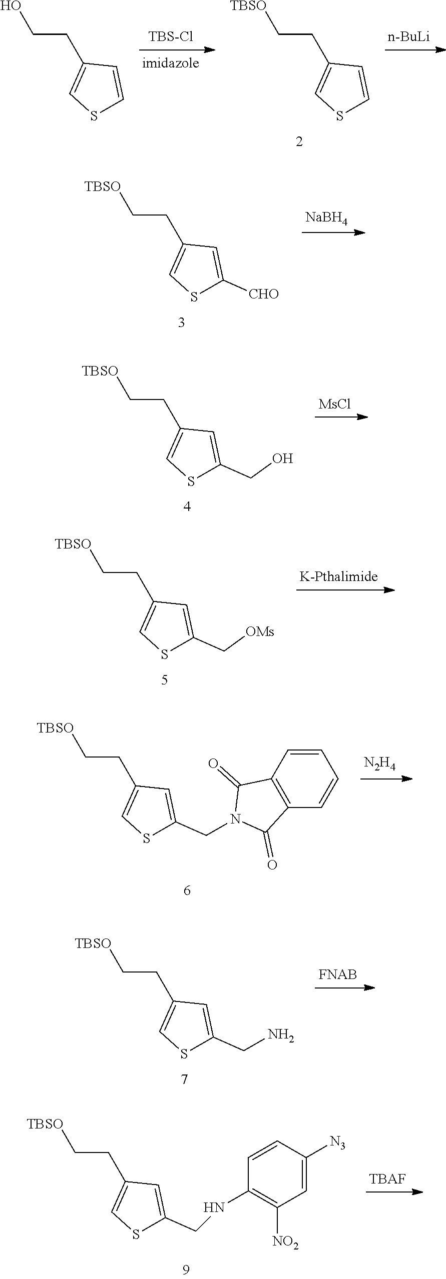

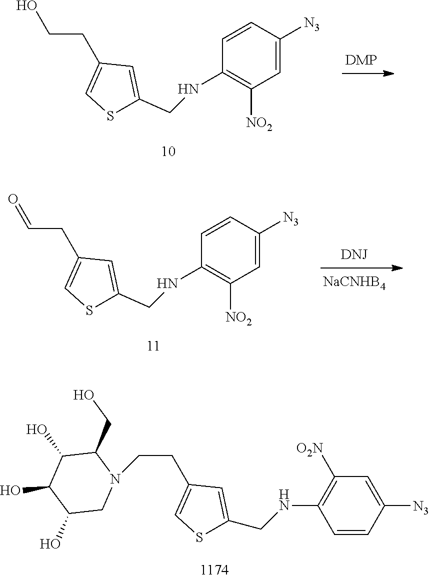

2-Thiopheneethanol (10.0 g), DCM (100 mL) mixed at 0° C., imidazole (1.5 eq), TBS-Cl (1.5 eq) added and increased to room temperature for 16 hours. Reaction was diluted with water and extracted with DCM. The organic layer was dried over anhydrous Na₂SO₄ and concentrated. The product was purified on silica [5% EtoAc:hexane] to afford 13 g of 2.

2 (8.0 g), THE (160 mL), n-BuLi (1.2 eq, 1.6 M in hexane) at −70° C. for 30 minutes, DMF (2.0 eq) added at −70° C. then increased to room temperature for 30 minutes. Reaction was diluted with saturated NH₄Cl and extracted with EtOAc. The organic layer was dried over anhydrous Na₂SO₄ and concentrated, and purified on silica [10% EtOAc:hexane] to afford 8 g of 3.

3 (8.0 g), MeOH (100 mL) at 0° C., NaBH₄ (2.0 eq) added then room temperature for 2 hours. Solvent was removed and residue diluted with water. The aqueous layer was extracted with EtOAc. The organic layer was dried over anhydrous Na₂SO₄ and concentrated. The material was purified on silica [10% EtoAc:hexane] to afford 4.0 g of 4.

4 (4.0 g), DCM (50 mL) at 0° C., TEA (3.0 eq), Ms-Cl (1.1 eq) maintained at 0° C. 15 minutes. Reaction was diluted with water and extracted with DCM. The organic layer was dried over anhydrous Na₂SO₄ and concentrated to afford 4.0 g of 5.

5 (4.0 g), DMF (30 mL), potassium phthalamide (5.0 eq) reacted at 70° C. for 16 hours. Reaction diluted with water, extracted with EtOAc, organic layer dried over anhydrous Na₂SO₄, concentrated, and purified on silica [10% EtOAc in hexane] to give 2.0 g of 6.

6 (2.0 g), EtOH (40 mL), 35% N₂H₄ (5.0 eq) mixed at room temperature for 16 hours. Solvent was removed and reaction mass diluted with water and extracted with EtOAc. The organic layer was dried over anhydrous Na₂SO₄ and concentrated to afford 1.1 g of 7.

Mixed 7 (1.0 g), FNAB (1.0 eq), 1,4-dioxane (30 mL), TEA (3.0 eq) at 80° C. for 16 hours. The solvent was removed and residue diluted with water. The aqueous layer was extracted with EtOAc and organic layer was dried over anhydrous Na₂SO₄, concentrated, and purified on silica [10% EtOAc in hexane] to give 600 mg of 9 (with 100 mg of 10).

Mixed 9 (600 mg), THE (15 mL) at 0° C., added TBAF (1.0 M in THF, 1.5 eq) then room temperature for 2 hours. The solvent was concentrated and diluted with water. The aqueous layer was extracted with EtOAc. The organic layer was dried over anhydrous Na₂SO₄, concentrated, and purified on silica [30% EtOAc in hexane] to afford 200 mg of 10.

10 (300 mg) and DCM (3 mL) at 0° C., added DMP (1.5 eq) then room temperature for 2 hours. The reaction was diluted with sat. NaHCO₃ and extracted with DCM. The organic layer was dried over anhydrous Na₂SO₄ and concentrated to afford 300 mg of 11.

11 (300 mg), MeOH (10 mL), DNJ (1.0 eq), NaCNBH₃ (1.5 eq), AcOH (1 drop) maintained at room temperature for 16 hours. The solvent was removed. Residue was purified by preparative HPLC to afford 30 mg of 1174.

### Example 66: Synthesis of Compound 1177 ((2R,3R,4R,5S)-1-[2-(1-{2-[(4-azido-2-nitrophenyl)amino]ethyl}-1H-indol-3-yl)ethyl]-2-(hydroxymethyl)piperidine-3,4,5-triol)

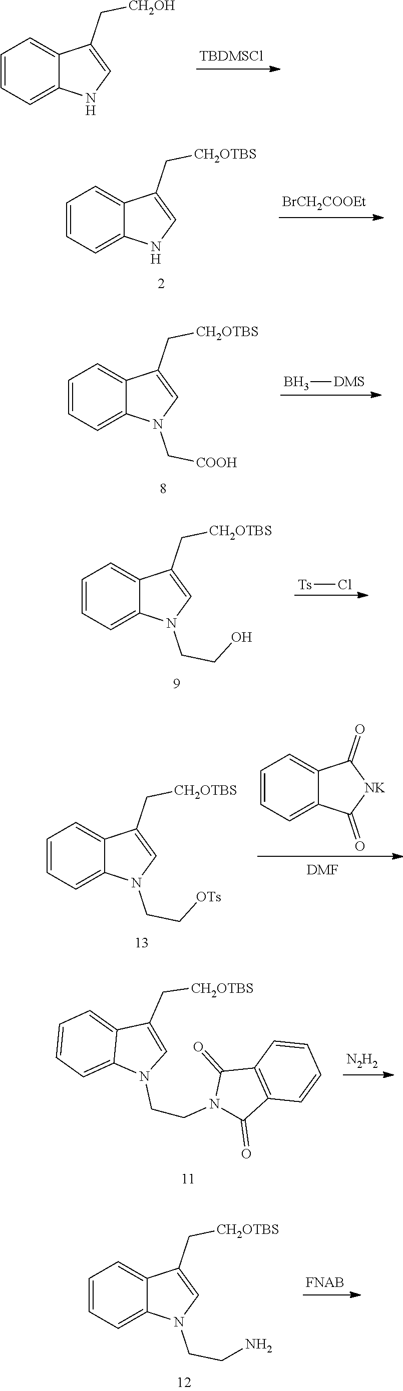

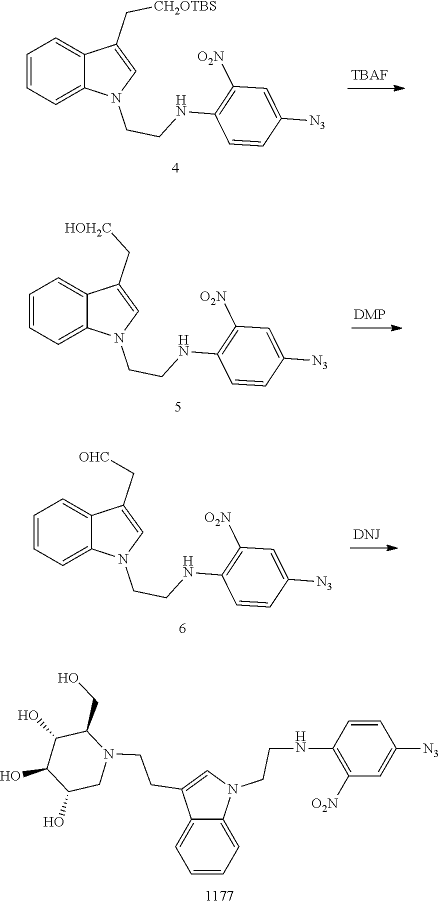

1 (5 g), DMF (50 mL) mixed at 0° C., imidazole (2.0 eq), TBDMSCl (1.2 eq) added and increased to room temperature. After 16 hours the reaction was quenched with water and extracted with EtOAc (2×100 mL). The organic layer was dried over Na₂SO₄, filtered, concentrated, and purified on silica [2% EtOAc:hexane] to afford 5.2 g of 2.

2 (5 g), NaH (2 eq) mixed at room temperature for 1 hourour ethyl bromoacetate (2 eq) added and mixed at room temperature for 1 hour. The reaction was quenched with ice-cold water and extracted with EtOAc (2×100 mL). The organic layer was washed with water and dried over Na₂SO₄, filtered, concentrated. The major product was extracted from water to provide 2 g of acid 8. (1.4 g of the ester of 8 was also purified on silica)

8 (1.8 g), THE (36 mL), 2.0 M BH₃·DMS in THE (3 eq) mixed at room temperature for 16 hours. The reaction was cooled to 0° C. and quenched with ice-cold water. The aqueous layer was extracted with EtOAc (2×100 mL). The organic layer was dried over Na₂SO₄, filtered, concentrated, and purified on silica [10% EtOAc:hexane] to afford 900 mg of 9.

9 (1.2 g), DCM (20 mL) mixed at 0° C., TEA (2 eq), tosyl chloride (1.5 eq), dimethylaminopyridine (DMAP, 0.1 eq) added and reacted at room temperature for 6 hours. The reaction was cooled to 0° C. and quenched with ice-cold water and extracted with DCM (2×100 mL). The organic layer was dried over Na₂SO₄, filtered, concentrated, and purified on silica [10% EtOAc:hexane] to afford 800 mg of 13.

13 (800 mg), DMF (5 mL), potassium phthalamide (5 eq) reacted at 80° C. for 16 hours. The reaction was cooled to room temperature and quenched with cool water. The aqueous layer was extracted with EtOAc (2×50 mL). The organic layer was dried over Na₂SO₄, filtered, concentrated, and purified on silica [10% EtOAc:hexane] to afford 600 mg of 11.

11 (600 mg), EtOH (2 mL), aq. 37% N₂H₂ (1 mlL maintained at room temperature for 16 hours. The reaction was concentrated and the residue dissolved in water and extracted with EtOAc (2×5 mL). The organic layer was dried over Na₂SO₄, filtered, concentrated to afford 400 mg of 12.

12 (400 mg), 1,4-dixoane (15 mL), 4-fluoro-3-nitroazidobenzene (1 eq), TEA (6 eq) reacted at 80° C. for 16 hours. The reaction was distilled and the residue dissolved in water and extracted with EtOAc (2×50 mL). Solvent was removed, and purified on silica [10% EtOAc:hexane] to afford 400 mg of 4.

4 (400 mg) in THE at 0° C., added 1.0 M TBAF in THE (1.5 mL) increased to room temperature for 3 hours. The reaction was dissolved in water and extracted with EtOAc (2×20 mL). The solvent was removed, and purified on silica [10% EtOAc-hexane] to afford 280 mg of 5.

Mixed 5 (210 mg) in DCM at 0° C., added DMP (1.5 eq), warmed to room temperature, quenched with water, extracted with DCM (2×20 mL), removed solvent to afford 200 mg of 6.

6 (200 mg), MeOH/DCM (1:1, 15 mL), DNJ (1 eq), AcOH (0.1 mL) mixed at 0° C. then NaCNBH₃ (1.5 eq) added and increased to room temperature. After 16 hours the reaction mass was concentrated then purified by preparative HPLC to afford 40 mg of 1177.

### Example 67: Synthesis of Compound 1178 ((2S,3S,4S,5R)-1-[2-(4-{2-[(4-azido-2-nitrophenyl)amino]ethyl}naphthalen-1-yl)ethyl]-2-(hydroxymethyl)piperidine-3,4,5-triol)

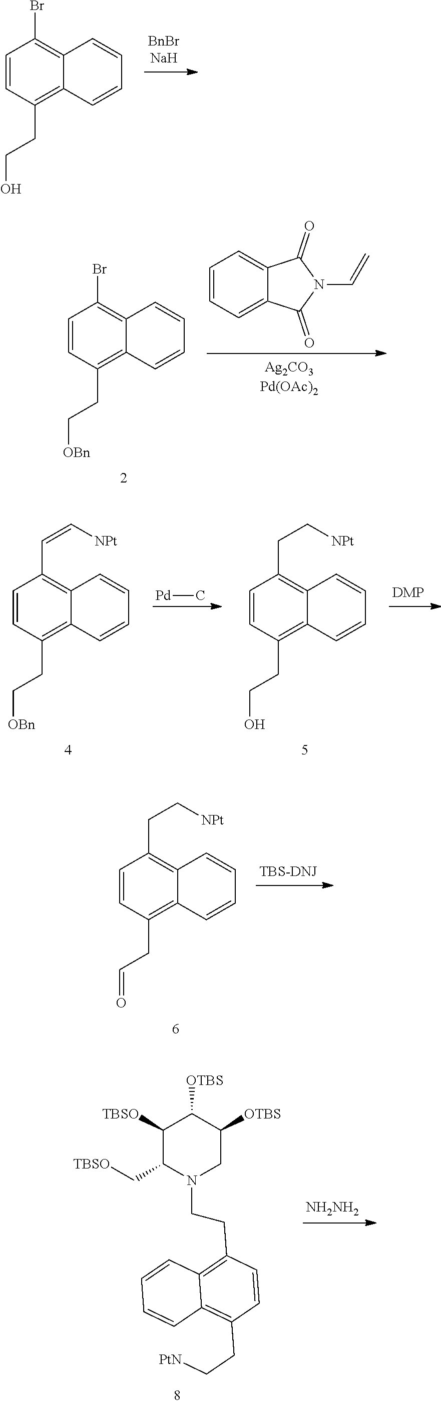

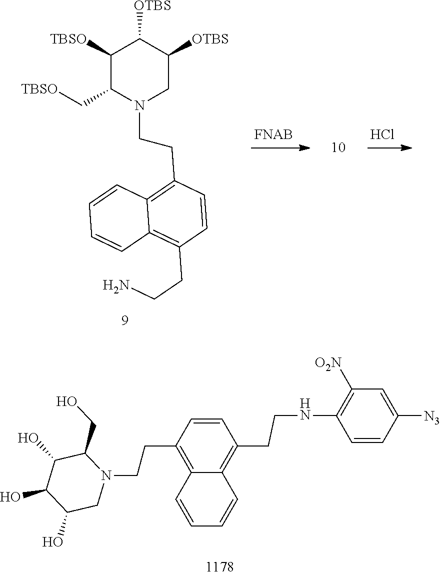 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCc1ccc(CCN)c2ccccc12 ; "FNAB" ; Cl ; [N-]=[N+]=Nc1ccc(NCCc2ccc(CCN3C[C@H](O)[C@@H](O)[C@H](O)[C@H]3CO)c3ccccc23)c([N+](=O)[O-])c1]

NaH (1.5 eq), THE (20 mL), 2-(4-bromonaphthalen-1-yl)ethan-1-ol (2 g) in THE (5 mL) at 0° C. for 1 hour benzyl bromide (1.2 eq) added at 0° C. then to room temperature for 16 hours. The reaction was quenched with ice-cold water and extracted with EtOAc (2×60 mL). The organic layer was washed with brine and dried over Na₂SO₄, filtered, concentrated. Material was purified on silica [4% EtOAc:hexane] to afford 2 g of 2.

2 (2.2 g), 1,1,1,3,3,3-hexafluoro-2-propanol (25 mL), 2-vinylisoindoline-1,3-dione (2 eq), Pd(OAc)₂ (0.1 eq), N-acetyl glycine (0.2 eq), Ag₂CO₃ (2 eq) reacted in sealed tube at 80° C. for 16 hours. The reaction mass was filtered on CELITE and washed with EtOAc. The organic layer was concentrated and purified on silica [7% EtOAc:hexane] to afford 1.6 g of 4.

4 (1.6 g), EtOAc (30 mL), 10% Pd/C (1.6 g, 50% wet), under H₂ at room temperature for 16 hours. The reaction mass was filtered on CELITE and washed with EtOAc. The organic layer was concentrated to afford 1 g of 5.

5 (800 mg), DCM (15 mL), DMP (1.5 eq) maintained at 0° C. for 2 hours. The reaction was quenched with aqueous NaHCO₃ and extracted with DCM (2×60 mL). The organic layer was filtered and washed with water, brine solution, dried over anhydrous Na₂SO₄, then concentrated to afford 900 mg of 6.

6 (900 mg), MeOH (20 mL), TBS-DNJ (1.2 eq), AcOH (0.5 mL) mixed at 0° C., NaCNBH₃ (1.5 eq) added then increased to room temperature and stirred 16 hours. The reaction was quenched with water and extracted with EtOAc (2×60 mL). The organic layer was washed with brine and dried over Na₂SO₄, filtered, concentrated, and purified on silica [7% EtOAc:hexane] to afford 800 mg of 8.

8 (800 mg), EtOH (20 mL), hydrazine hydrate (2.5 eq, 75% solution) mixed at room temperature, stirred 16 hours then added water and extracted with EtOAc (2×60 mL). The organic layer was washed with brine and dried over Na₂SO₄, filtered, concentrated to afford 600 mg of 9.

9 (600 mg), 1,4-dioxane (20 mL), FNAB (1.1 eq), TEA (3 eq+1 eq) was refluxed 6 hours. Removed solvent, added water, extracted with EtOAc (2×50 mL). The organic layer was washed with brine and dried over Na₂SO₄, filtered, concentrated. Residue was purified on silica [2% EtOAc:hexane] to afford 300 mg of 10.

Mixed 10 (300 mg), 1,4-dioxane (20 mL), 4.0 M HCl in 1,4-dioxane (5.0 mL) at 0° C., stirred 5 hours at 10° C. Removed solvent, preparative HPLC purification afforded 11 mg of 1178.

### Example 68: Synthesis of Compound 1179 ((2R,3R,4R,5S)-1-[2-(5-{2-[(4-azido-2-nitrophenyl)amino]ethyl}thiophen-3-yl)ethyl]-2-(hydroxymethyl)piperidine-3,4,5-triol)

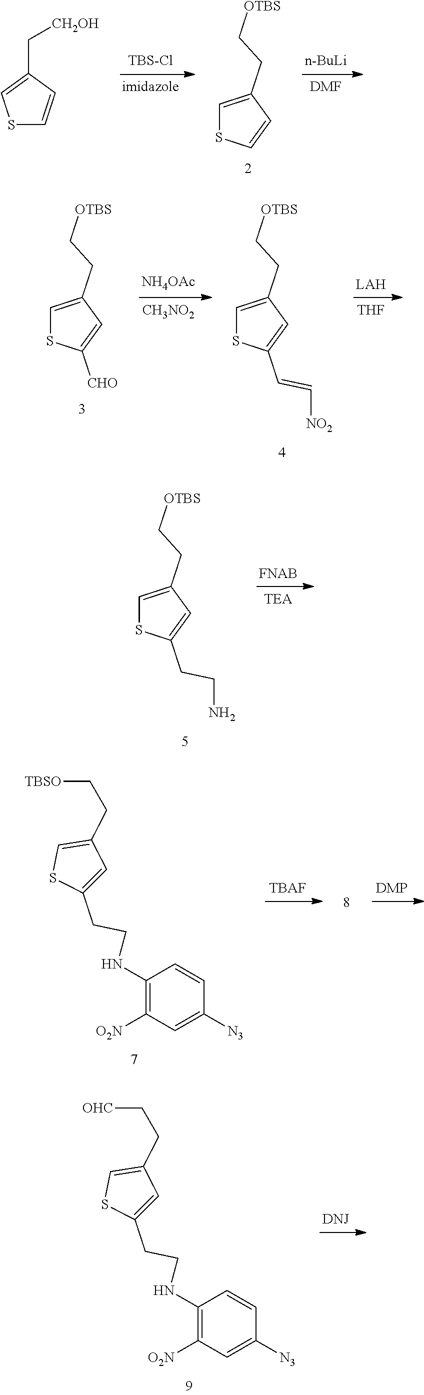

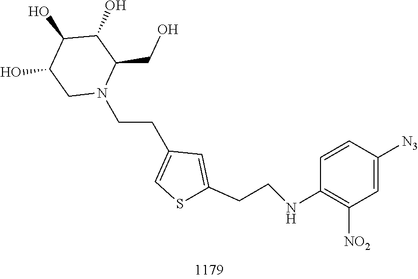 [SMILES: [N-]=[N+]=Nc1ccc(NCCc2cc(CCN3C[C@H](O)[C@@H](O)[C@H](O)[C@H]3CO)cs2)c([N+](=O)[O-])c1]

2-(Thiophen-3-yl)ethan-1-ol (1.0 g), DCM (15 mL) mixed at 0° C., imidazole (1.5 eq), TBS-Cl (1.5 eq) added and increased to room temperature. After 16 hours reaction was diluted with water and extracted with DCM. The organic layer was dried over anhydrous Na₂SO₄ and concentrated. Product was purified on silica [5% EtoAc:hexane] to afford 1.2 g of 2.

2 (1.0 g), THE (25 mL), n-BuLi (1.0 eq, 1.6 M in hexane), mixed at −70° C. 30 minutes, DMF (2.0 eq) added at −70° C. then increased to room temperature for 30 minutes. Diluted with saturated NH₄Cl and extracted with EtOAc, dried the organic layer over anhydrous Na₂SO₄, concentrated, and purified on silica [10% EtOAc:hexane] to afford 500 mg of 3.

3 (500 mg), nitromethane (3 mL), NH₄OAc (5.0 eq) reacted at 100° C. for 1 hour. Solvent was removed. Residue was purified on silica [5% EtOAc-hexane] to afford 400 mg of 4.

LAH (8.5 eq), THE (10 mL), 4 (400 mg), THE (6 mL) was refluxed 1 hour. Reaction was quenched with saturated NaF (1 mL) and filtered through CELITE. Drying the filtrate over anhydrous Na₂SO₄ and concentrating gave 300 mg of 5.

5 (300 mg), 1,4-dioxane (2 mL), FNAB (1.0 eq), TEA (3.0 eq) was refluxed 16 hours. Solvent was removed and residue quenched with water and extracted with EtOAc. The organic layer was dried over anhydrous Na₂SO₄, concentrated, and purified on silica [10% EtOAc in hexane] to afford 300 mg of 7.

7 (300 mg), THE (5 mL) mixed at 0° C., added TBAF (1.0 M in THF, 1.5 eq), maintained at room temperature 2 hours. Solvent was removed and quenched with water and extracted with EtOAc. The organic layer was dried over anhydrous Na₂SO₄, concentrated, and purified on silica [25% EtOAc in hexane] to afford 200 mg of 8.

8 (190 mg), DCM (10 mL) mixed at 0° C., added DMP (1.5 eq), maintained at room temperature for 1 hour. Reaction was diluted with DCM and saturated NaHCO₃. The organic layer was separated and dried over anhydrous Na₂SO₄ then concentrated to provide 9.

9 (190 mg), MeOH (1 mL), DNJ (1.0 eq), AcOH (1 drop), NaCNBH₃ (1.5 eq) maintained at room temperature 16 hours. The solvent was removed. After preparative HPLC purification, 14 mg of 1179 was obtained (by ¹H-NMR included ˜12% of the 2,3-substituted isomer at the thiophene ring).

### Example 69: Synthesis of Compounds 1182 ((2R,3R,4R,5S)-2-(hydroxymethyl)-1-[6-({3-methoxy-5-[(1,2-oxazolidin-2-yl)methyl]phenyl}amino)hexyl]piperidine-3,4,5-triol) and 1183 ((2R,3R,4R,5S)-1-[6-({3-chloro-5-[(1,2-oxazolidin-2-yl)methyl]phenyl}amino)hexyl]-2-(hydroxymethyl)piperidine-3,4,5-triol)

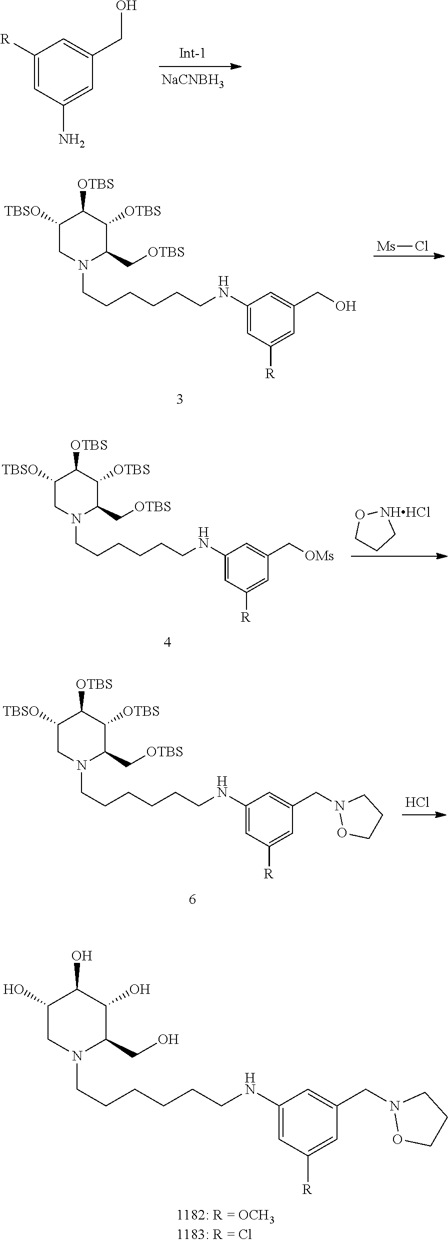

Preparation of 1182 (2R,3R,4R,5S)-2-(hydroxymethyl)-1-[6-({3-methoxy-5-[(1,2-oxazolidin-2-yl)methyl]phenyl}amino)hexyl]piperidine-3,4,5-triol: Mixed Int-1 (Example 1, 2.5 g), MeOH (70 mL) at 0° C., increased to room temperature, added (3-amino-5-methoxyphenyl)methanol (0.8 eq), NaCNBH₃ (1.5 eq), AcOH (cat.), maintained at room temperature 16 hours. Reaction was quenched with water and diluted with EtOAc (50 mL). The organic layer was separated, washed with brine and dried over Na₂SO₄. The organic layer was concentrated. Material was purified on silica [eluted with EtOAc] to afford 1 g of 3.

3 (850 mg), DCM (20 mL), Et₃N (4 eq), MsCl (2 eq) reacted −50° C. for 1 hour. The reaction was quenched with water and diluted with DCM (10 mL). The organic layer was separated, washed with brine and dried over Na₂SO₄. The organic layer was concentrated to afford 700 mg of 4.

4 (700 mg), THE (8 mL), DIPEA (8 mL), isoxazolidine hydrochloride (1.5 eq) mixed at room temperature and raised to 120° C. After 48 hours the reaction was quenched with water and diluted with DCM (30 mL). The organic layer was separated, washed with brine and dried over Na₂SO₄. The organic layer was concentrated and purified on silica [10% EtOAc in hexane] to afford 200 mg of 6 as colorless thick syrup.

6 (200 mg), MeOH (5 mL), 4.0 MHCl in 1,4-dioxane (2 mL) mixed at 0° C. then increased to room temperature for 1 hour. The reaction mass was concentrated. Preparative HPLC purification afforded 28 mg of 1182 as colorless thick syrup.

Preparation of 1183 (2R,3R,4R,5S)-1-[6-({3-chloro-5-[(1,2-oxazolidin-2-yl)methyl]phenyl}amino)hexyl]-2-(hydroxymethyl)piperidine-3,4,5-triol: Mixed Int-1 (3.75 g) in MeOH (70 mL) at 0° C., added (3-amino-5-chlorophenyl)methanol (0.8 eq), NaCNBH₃ (1.5 eq), AcOH (cat.), raised to room temperature for 16 hours. The reaction was quenched with water and diluted with EtOAc (50 mL). The organic layer was separated, washed with brine and dried over Na₂SO₄. The organic layer was concentrated and purified on silica [100% EtOAc] to afford 1 g of 3.

3 (750 mg), DCM (15 mL), Et₃N (4 eq), mesyl chloride (2 eq) reacted at 0° C. for 1 hour. The reaction was quenched with water and diluted with DCM (10 mL). The organic layer was separated, washed with brine, dried over Na₂SO₄, concentrated to afford 700 mg of 4.

4 (700 mg), THF (10 mL), DIPEA (10 mL), isoxazolidine HCl (1.5 eq) mixed at room temperature then at 80° C. for 16 hours. The reaction was quenched with water and diluted with DCM (30 mL). The organic layer was separated, washed with brine, dried over Na₂SO₄, concentrated, and purified on silica [100% EtOAc] to afford 200 mg of 6.

To 6 (200 mg) in MeOH (5 mL) at 0° C. added 4.0 M HCl in 1,4-dioxane (2 mL), increased to room temperature for 1 hour. Volatiles were concentrated and the material was purified by preparative HPLC to afford 46 mg of 1183 as colorless thick syrup.

### Example 70: Synthesis of Compound 1188 ((2R,3R,4R,5S)-1-[2-(2-{2-[(4-azido-2-nitrophenyl)amino]ethyl}pyrimidin-4-yl)ethyl]-2-(hydroxymethyl)piperidine-3,4,5-triol)

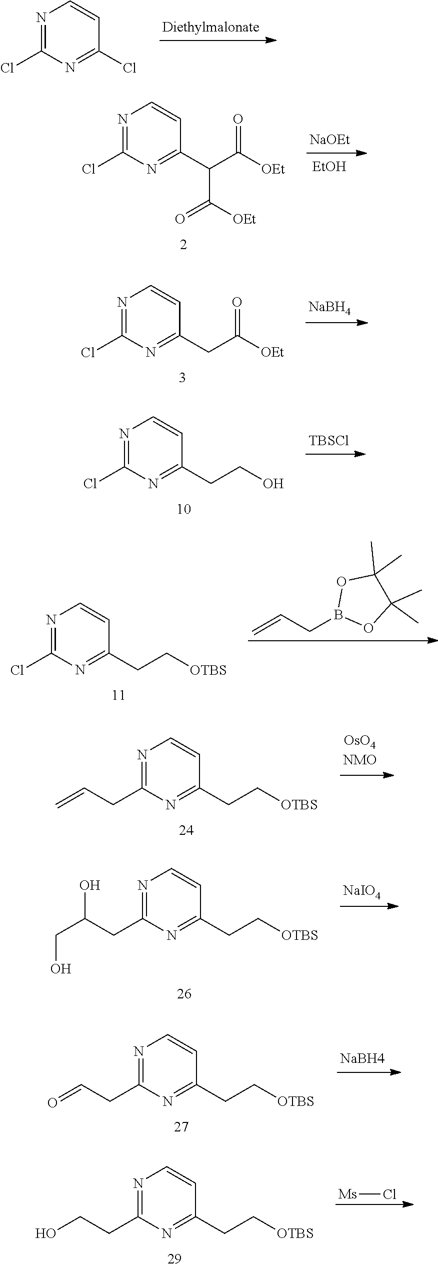

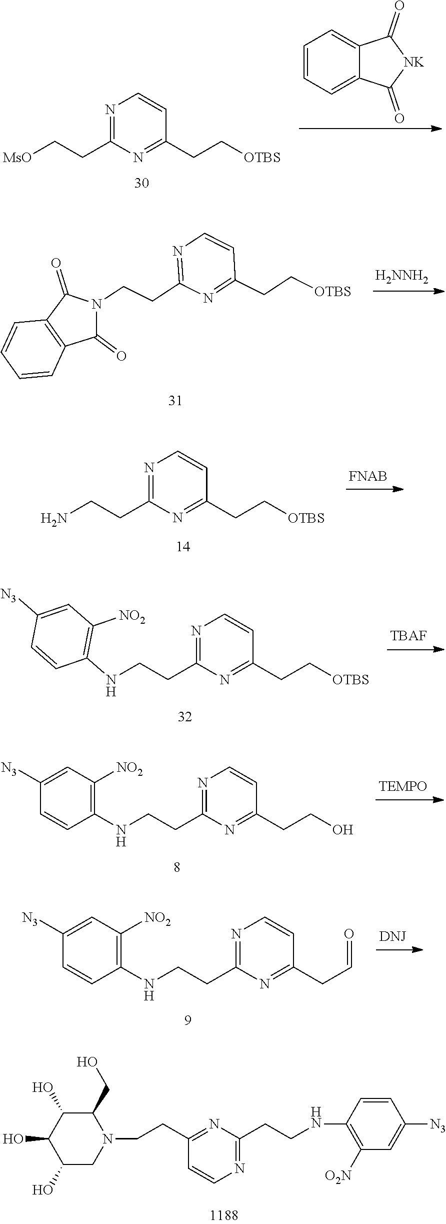

To diethylmalonate (69 g) in THE (1.3 L) at room temperature added 2,4-dichloro-pyrimidine (0.66 eq), NaH (2 eq), reacted at 2 hours at 100° C. Cooled to room temperature, quenched with ice-cold water, extracted with EtOAc (2×1 L). Organic layer was dried over Na₂SO₄, filtered, concentrated, and purified on silica [7% EtOAc in hexane] to afford 40 g of 2.

2 (40 g), EtOH (1.2 L), NaOEt (0.1 eq) was refluxed 42 hours. The reaction was cooled to room temperature and concentrated. Residue was diluted with ice-cold water and extracted with EtOAc (2×2 L). The organic layer was dried over Na₂SO₄, filtered, concentrated, purified on silica [7% EtOAc in hexane] to afford 20 g of 3.

Mixed 3 (20 g), THE (400 mL), EtOH (400 mL), CaCl₂) (2 eq), NaBH₄ (4 eq) at 0° C., increased to room temperature. After 2 hours the reaction was quenched with saturated aqueous NH₄Cl (800 mL) and extracted with EtOAc (2×1 L). The organic layer was washed with brine, dried over Na₂SO₄, filtered, concentrated to afford 15 g of 10.

Mixed 10 (15 g), DCM (500 mL) at 0° C., added imidazole (2.5 eq), TBS-Cl (1.5 eq) mixed 16 hours at room temperature. The reaction was quenched with ice-cold water (800 mL) and extracted with DCM (2×500 mL). The organic layer was washed with brine and dried over Na₂SO₄, filtered, concentrated, purified on silica [3% EtOAc-hexane] to afford 10.3 g of 11.

11 (6 g), dioxane:H₂O (1:4, 200 mL), 2-allyl boronate (2 eq), Na₂CO₃ (2 eq), degassed 15 minutes N₂, added Pd(dppf)Cl₂ (0.05 eq) reacted at 80° C. for 16 hours. The reaction was diluted with water and extracted with EtOAc (2×200 mL). The organic layer was dried over Na₂SO₄, filtered, concentrated, purified on silica [20% EtOAc in hexane] to afford 2.6 g of 24.

24 (2.6 g), N-methyl-morpholine-N-oxide (NMO, 4 eq), 4% OsO₄ in H₂O (0.05 eq), acetone:H₂O (34 mL:17 mL) reacted at room temperature for 5 hours. The reaction was quenched with saturated aqueous Na₂SO₃ and extracted with EtOAc (2×100 mL). The organic layer was dried over Na₂SO₄, filtered, concentrated to afford 2 g of 26.

26 (2 g), NaIO₄ (1.3 eq), DCM:H₂O (1:1 200 mL) reacted at 0° C. for 10 minutes. The reaction was neutralized by saturated aqueous NaHCO₃ and extracted with DCM (2×100 mL). The organic layer was dried over Na₂SO₄, filtered, concentrated to afford 1.6 g of 27.

27 (1.6 g), MeOH (50 mL), NaBH₄ (1.3 eq) reacted at room temperature, 1 hour. The reaction was concentrated. The residue was dissolved in water, extracted with EtOAc (2×100 mL). The organic layer was dried over Na₂SO₄, filtered, concentrated to afford 1.2 g of 29.

29 (1.2 g), mesyl chloride (1.3 eq), DCM (30 mL), TEA (2 eq) reacted at room temperature 30 minutes. The reaction was quenched with ice-cold water and extracted with DCM (2×20 mL). The organic layer was dried over Na₂SO₄, filtered, concentrated, purified on silica [20% EtOAc in Hexane] to afford 1.2 g of 30.

Reacted 30 (1.2 g), potassium phthalimide (10 eq), in DMF at 70° C., 6 hours. Quenched with ice-cold water, extracted with DCM (2×20 mL), dried the organic layer over Na₂SO₄, filtered, concentrated, purified on silica [20% EtOAc-hexane] to afford 1.2 g of 31.

31 (1.2 g), EtOH (100 mL), 35% N₂H₂ in H₂O (15 mL) mixed at room temperature for 16 hours. The reaction was concentrated, residue dissolved in water and extracted with EtOAc (2×100 mL). The organic layer was dried over Na₂SO₄, filtered, concentrated to afford 600 mg of 14.

14 (600 mg), FNAB (1 eq), TEA (4 eq), 1,4-dioxane (10 mL) reacted at 80° C. 16 hours. The reaction was cooled to room temperature, concentrated, and purified on silica [50% EtOAc in hexane] to afford 500 mg of 32.

32 (500 mg), THE (5 mL), 1.0 M TBAF in THE (1.5 eq) held 2 hours at room temperature. The reaction was quenched with ice-cold water and extracted with EtOAc (2×10 mL). The organic layer was dried over Na₂SO₄, filtered, concentrated, purified on silica [80% EtOAc-hexane] to afford 280 mg of 8.

8 (250 mg), DCM (21.6 mL), H₂O (21.6 mL), TEMPO (0.22 eq), NaBr (1.6 eq), NaOCl (1.8 eq), NaHCO₃ (3.5 eq) held 15 minutes at 0° C. Reaction was quenched with 30 mL ice-cold water and extracted with 20 mL DCM. Organic layer was washed with brine, dried over Na₂SO₄, filtered, concentrated to afford 250 mg of 9.

Mixed 9 (250 mg), DCM/MeOH (1:1, 30 mL), DNJ (2 eq), AcOH (0.1 mL), NaCNBH₃ (1.5 eq) 16 hours at room temperature. Preparative HPLC purification of combined batches provided 10 mg 1188 as a pale red solid.

### Example 71: Synthesis of Compound 1191 ((2R,3R,4R,5S)-1-[2-(4-{2-[(4-azido-2-nitrophenyl)amino]ethyl}-1H-pyrazol-1-yl)ethyl]-2-(hydroxymethyl)piperidine-3,4,5-triol)

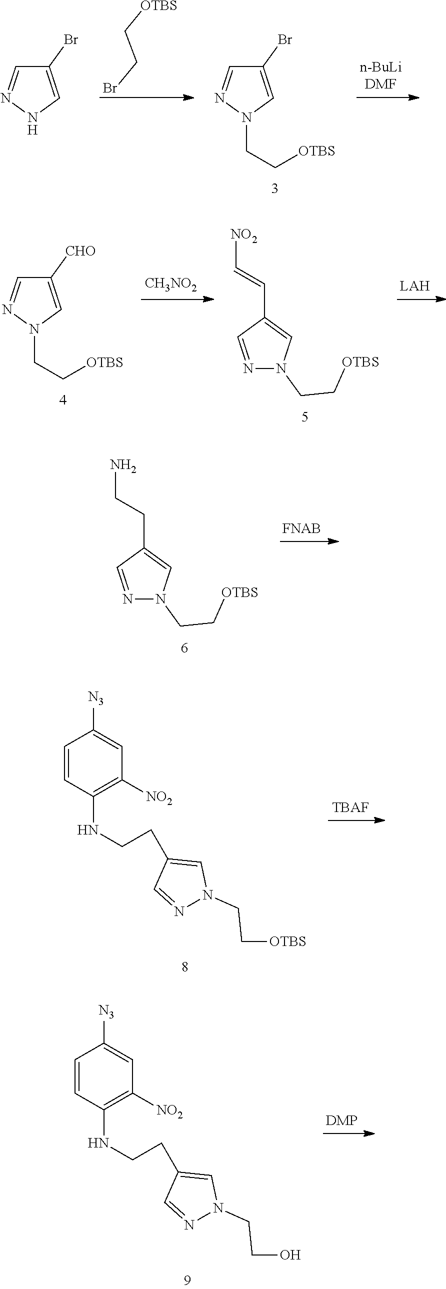

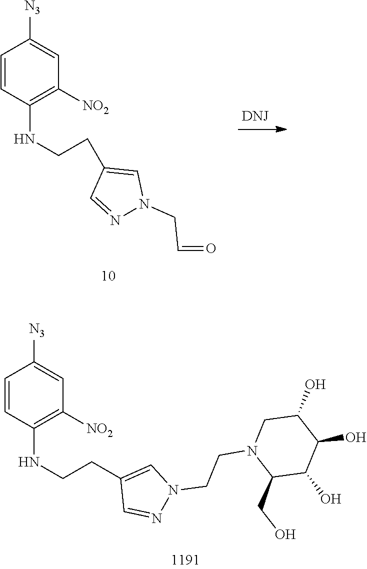 [SMILES: [N-]=[N+]=Nc1ccc(NCCc2cnn(CC=O)c2)c([N+](=O)[O-])c1 ; "DNJ" ; [N-]=[N+]=Nc1ccc(NCCc2cnn(CCN3C[C@H](O)[C@@H](O)[C@H](O)[C@H]3CO)c2)c([N+](=O)[O-])c1]

To 1 (7 g) in ACN (150 mL) at room temperature added K₂CO₃ (3.0 eq), 2-bromoethanol TBS ether (1.2 eq) at room temperature and heated at 80° C. for 12 hours. Reaction mass was diluted with water and extracted with EtOAc. The organic layer was dried over anhydrous Na₂SO₄, concentrated, and purified on silica [5% EtOAc in hexane] to afford 12 g of 3.

Mixed 3 (12 g), THE (220 mL), n-BuLi (1.6 M in hexane, 1.3 eq) at −78° C. for 1 hour, added DMF (5.0 eq) at −78° C. for 1 hour. Quenched with saturated NH₄Cl and extracted with EtOAc. The organic layer was washed with water and brine, dried over anhydrous Na₂SO₄, concentrated, and purified on silica [20% EtOAc in hexane] to afford 6 g of 4.

4 (6.0 g), nitromethane (10 eq), NH₄OAc (5.0 eq) reacted at 100° C. for 1 hour. Reaction mass was concentrated and purified on silica [20% EtOAc-hexane] to afford 4.5 g of 5.

5 (1 g), 10% Pd/C (50% wet, 500 mg), MeOH (20 mL) stirred under H₂ at room temperature for 16 hours, then worked up to remove catalyst. In a separate reaction, mixed 5a (2 g), LAH (8.5 eq), THE (50 mL) at 0° C. then refluxed 2 hours. LCMS of these batches showed 50-97% of TBS-de-protected 6 which were taken to amination without further purification.

6 (800 mg with low level of TBS ether), FNAB (1.0 eq), 1,4-dioxane (20 mL), TEA (3.0 eq) mixed at room temperature then reacted at 80° C. for 16 hours. Reaction mass was diluted with water, extracted with EtOAc, organic layer was dried over anhydrous Na₂SO₄, concentrated, and purified on silica [5% MeOH in DCM] to afford 500 mg of 9 as brown thick syrup. A similar reaction was carried out on 6 which was predominantly OTBS ether, to give 8.

To deprotect residual TBS ether, 8 (230 mg), THE (2.5 mL), 1M TBAF in THE (1.5 eq) was mixed at 0° C. then at room temperature for 4 hours. Quenched with ice-cold water and extracted with EtOAc. The combined organic layer was dried over Na₂SO₄, concentrated, and purified on silica [60% EtOAc:hexane] to give 100 mg of 9 as brown thick syrup.

9 (200 mg, combined), DCM (50 mL), DMP (1.5 eq) was mixed at 0° C. then room temperature for 1 hour, quenched with saturated NaHCO₃ and extracted with DCM. The organic layer was dried over anhydrous Na₂SO₄ and concentrated to afford 150 mg of 10.

Mixed 10 (150 mg), DNJ (1 eq), AcOH (cat.) at 0° C., added NaCNBH₃ (1.5 eq), mixed at room temperature for 16 hours. Combined batches preparative HPLC purification gave 30 mg of 1191.

### Example 72: Synthesis of Compound 1186 ((2R,3R,4R,5S)-1-[2-(5-{2-[(4-azido-2-nitrophenyl)amino]ethyl}hiophen-2-yl)ethyl]-2-(hydroxymethyl)piperidine-3,4,5-triol)

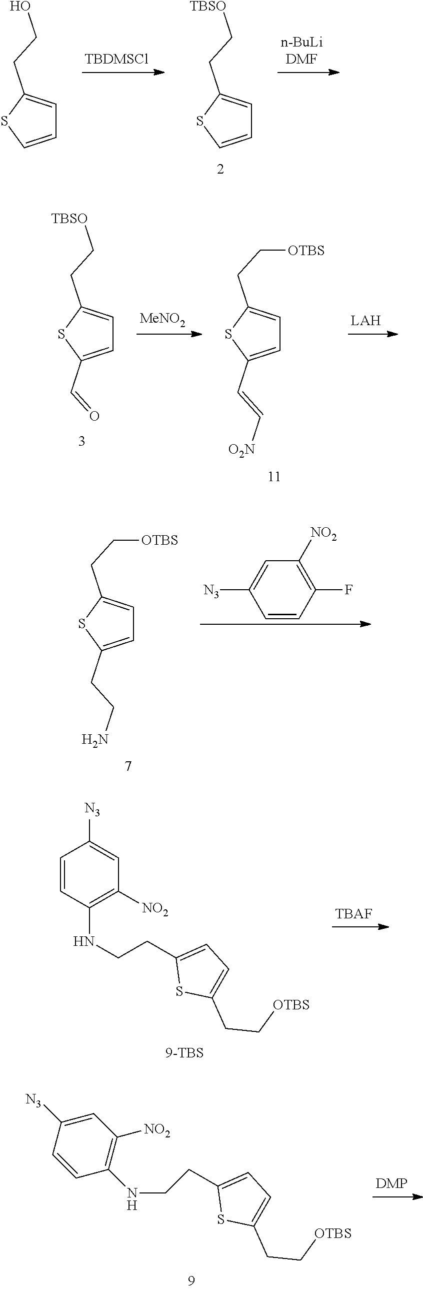

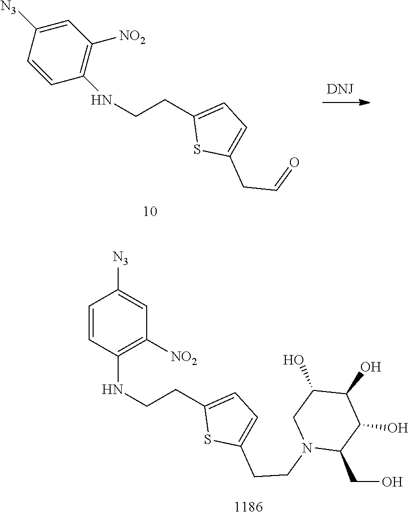 [SMILES: [N-]=[N+]=Nc1ccc(NCCc2ccc(CC=O)s2)c([N+](=O)[O-])c1 ; "DNJ" ; [N-]=[N+]=Nc1ccc(NCCc2ccc(CCN3C[C@H](O)[C@@H](O)[C@H](O)[C@H]3CO)s2)c([N+](=O)[O-])c1]

To (thiophen-2-yl)ethan-1-ol (15 g) in DCM (300 mL) at 0° C. added imidazole (1.5 eq), TBDMSCl (1.2 eq), increased to room temperature for 16 hours. The reaction was quenched with ice-cold water (200 mL) and extracted with DCM (2×200 mL). The organic layer was washed with brine, dried over Na₂SO₄, filtered, concentrated, and the residue purified on silica [1-3% EtOAc in hexane] to afford 25 g of 2 as colorless oil.

2 (25 g), THE (500 mL) at −78° C. added n-BuLi (2.5 M in hexane, 1.2 eq), held 1 hour, added DMF (1.5 eq) then maintained at room temperature for 16 hours. The reaction was quenched with ice-cold water (250 mL) and extracted with EtOAc (2×250 mL). The organic layer was washed with brine, dried over Na₂SO₄, filtered, concentrated, and the material purified on silica [2% EtOAc in hexane] to afford 15 g of 3 as colorless oil.

3 (10 g), CH₃NO₂ (150 mL), NH₄OAc (5 eq) mixed at room temperature then 80° C. for 4 hours. The reaction was quenched with ice-cold water (100 mL) and extracted with EtOAc (2×100 mL). The organic layer was washed with brine, dried over Na₂SO₄, filtered, concentrated, and then material purified on silica [2% EtOAc in hexane] to afford 6 g of 11 as yellow oil.

11 (7.5 g), LAH (8.5 eq), THE (150 mL) mixed at 0° C., warmed to 50° C. for 30 minutes, quenched with saturated NaF solution, filtered through a CELITE bed, washed with EtOAc, the filtrate dried over Na₂SO₄, filtered, and concentrated to give 5.5 g of 7.

Mixed 7 (5.5 g), FNAB (0.6 eq), 1,4-dioxane (110 mL), TEA (3 eq) at room temperature then at 80° C. for 16 hours. Quenched with ice-cold water (40 mL) and extracted with EtOAc (2×60 mL). The organic layer was washed with brine, dried over Na₂SO₄, filtered, concentrated, and purified on silica [5% EtOAc in hexane] to afford 1.5 g of 9-TBS as yellow oil.

Mixed 9-TBS (1.5 g), THE (30 mL), 1.0 M TBAF in THE (1 eq) at 0° C. then 2 hours at room temperature. The reaction was quenched with ice-cold water (20 mL) and extracted with EtOAc (2×30 mL). The organic layer was washed with brine, dried over Na₂SO₄, filtered, concentrated, and purified on silica [40% EtOAc in hexane] to afford 300 mg of 9 as yellow oil.

Mixed 9 (300 mg), DCM (10 mL), DMP (1.5 eq) at 0° C. then at room temperature for 2 hours. Quenched with saturated NaHCO₃ solution (10 mL), extracted with DCM (2×10 mL), washed organic layer with brine, dried over Na₂SO₄, filtered, concentrated to give 320 mg of 10.

Mixed 10 (320 mg), MeOH (10 mL), DNJ (0.8 eq), AcOH (cat.), NaCNBH₃ (1.5 eq) at 0° C. then at room temperature for 16 hours. Reaction mass was concentrated and preparative HPLC purification gave 42 mg of 1186 as orange-red solid.

### Example 73: Synthesis of Compound 1187 ((2R,3R,4R,5S)-1-[2-(6-{2-[(4-azido-2-nitrophenyl)amino]ethyl}pyrazin-2-yl)ethyl]-2-(hydroxymethyl)piperidine-3,4,5-triol)

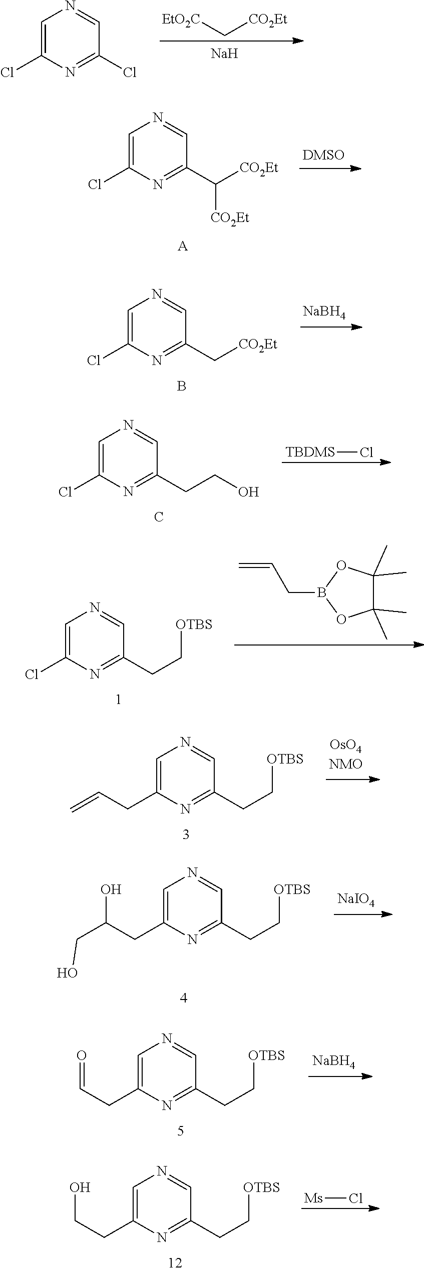

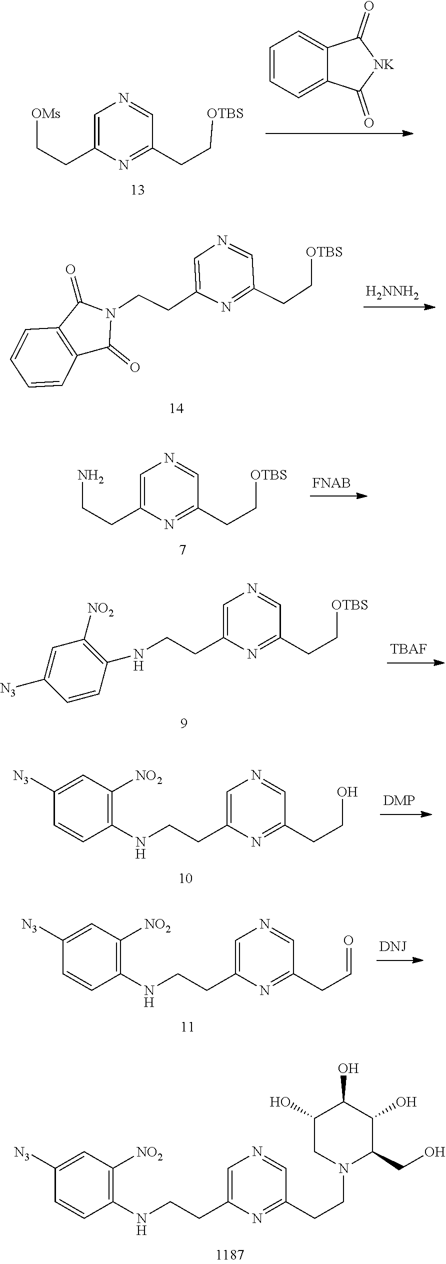

To diethylmalonate (30 g) in THE (120 mL) at room temperature added NaH (2 eq), 2,5-dichloro-pyrazine (0.47 eq) reacted at 75° C. The reaction was cooled to room temperature and quenched with ice-cold water and extracted with EtOAc (2×1 L). The organic layer was dried over Na₂SO₄, filtered, concentrated and reduced pressure. The material was purified on silica [7% EtOAc in hexane] to afford 20 g of A.

A (20 g), DMSO (700 mL), H₂O (700 mL), NaCl (8 eq) maintained at 120° C. for 24 hours. The reaction was cooled to room temperature and extracted with EtOAc (2×800 mL). The organic layer was washed with water and dried over Na₂SO₄, filtered, concentrated and reduced pressure to afford residue which was purified by COMBIFLASH to afford 7 g of B.

B (4.5 g), THE (40 mL), EtOH (40 mL), MeOH (6 mL), CaCl₂) (2 eq), NaBH₄ (4 eq) mixed at 0° C., then at room temperature for 2 hours. The reaction was quenched with ice-cold saturated aqueous NH₄Cl (500 mL) and extracted with EtOAc (2×200 mL). The organic layer was washed with brine and dried over Na₂SO₄, filtered, concentrated to afford 2.5 g of C.

To C (2.5 g) in DCM (100 mL) at 0° C. added imidazole (2.5 eq), TBDMS-Cl (1.2 eq), reacted at room temperature for 16 hours. The reaction was quenched with ice-cold water and extracted with DCM (2×100 mL). The organic layer was washed with brine, dried over Na₂SO₄, filtered, concentrated, purified on silica [5% EtOAc in hexane] to afford 2 g of 1.

1 (2 g), dioxane:H₂O (1:4, 84 mL), 2-allyl boronate (2 eq), Na₂CO₃ (2 eq), degassed 15 minutes N₂, added Pd(dppf)Cl₂ (0.05 eq) reacted at 80° C. 16 hours. The reaction was diluted with water and extracted with EtOAc (2×100 mL). The organic layer was dried over Na₂SO₄, filtered, concentrated. Obtained material was purified on silica [20% EtOAc in hexane] to afford 1.3 g of 3.

3 (1.3 g), NMO (4 eq), 4% OsO₄ in H₂O (0.05 eq), acetone:H₂O (4:1, 40 mL) maintained at room temperature 48 hours. The reaction was quenched with sat. aq. Na₂SO₃ and extracted with EtOAc (2×100 mL). The organic layer was dried over Na₂SO₄, filtered, concentrated to give 4.

4 (1.4 g), DCM:H₂O (1:1, 36 mL), NaIO₄ (1.3 eq) reacted at 0° C. 30 minutes. The reaction was neutralized by saturated aqueous NaHCO₃ and extracted with DCM (2×100 mL). The organic layer was dried over Na₂SO₄, filtered, concentrated to afford 1.3 g of 5.

5 (1.3 g), MeOH (50 mL), NaBH₄ (1.3 eq) reacted at room temperature 1 hour. The reaction was concentrated. The residue was dissolved in water then extracted with EtOAc (2×100 mL). The organic layer was dried over Na₂SO₄, filtered, concentrated to afford 1.2 g of 12.

12 (1.2 g), mesyl chloride (1.3 eq), DCM (30 mL), TEA (2 eq) reacted at room temperature 30 minutes. The reaction was quenched with ice-cold water and extracted with DCM (2×50 mL). The organic layer was dried over Na₂SO₄, filtered, concentrated, and the residue purified on silica [20% EtOAc in hexane] to afford 1.4 g of 13.

13 (1.4 g) and potassium phthalimide (4 eq) in DMF (10 mL) reacted at 70° C. 16 hours. The reaction was quenched with ice-cold water and extracted with DCM (2×100 mL). The organic layer was dried over Na₂SO₄, filtered, concentrated, and the residue purified on silica [20% EtOAc in hexane] to afford 1 g of 14.

Mixed 14 (1 g), EtOH (50 mL), 35% N₂H₂·H₂O (5 mL) at room temperature 16 hours then concentrated then dissolved residue in ice-cold water and extracted with EtOAc (2×100 mL). The organic layer was dried over Na₂SO₄, filtered, concentrated to afford 600 mg of 7.

7 (600 mg), 1,4-dioxane (30 mL), TEA (5 eq), FNAB (1 eq) reacted at 80° C. The reaction was concentrated. The residue was dissolved in ice-cold water and extracted with EtOAc (2×60 mL). The organic layer was dried over Na₂SO₄, filtered, concentrated, and the residue purified on silica [20% EtOAc in hexane] to afford 700 mg of 9.

9 (700 mg), 1.0 M TBAF in THE (1.5 eq), THE (3 mL) reacted at room temperature 2 hours. The reaction was quenched with ice-cold water and extracted with EtOAc (2×50 mL). The organic layer was dried over Na₂SO₄, filtered, concentrated, and the residue purified on silica [80% EtOAc in hexane] to afford 300 mg of 10.

10 (320 mg), DMP (1.5 eq+1 eq+1 eq) (3 lots added at 30 minutes intervals), DCM (60 mL) at 0° C. then held at room temperature for 2 hours. The reaction was quenched with ice-cold water (15 mL) and extracted with DCM (2×15 mL). The organic layer was washed with saturated aqueous NaHCO₃ and dried over Na₂SO₄, filtered, concentrated to afford 320 mg of 11.

11 (320 mg), DNJ (1 eq), MeOH (20 mL), DCM (20 mL), AcOH (0.2 mL), NaCNBH₃ (1.5 eq) reacted at room temperature 16 hours. The reaction mass was concentrated. Purification of combined batches by preparative HPLC afforded 42 mg of 1187.

### Example 74: Synthesis of Compound 1184 5-(pyrimidin-2-yl)-2-({6-[(2R,3R,4R,5S)-3,4,5-trihydroxy-2-(hydroxymethyl)piperidin-1-yl]hexyl}amino)benzaldehyde and 1201 (2R,3R,4R,5S)-1-(6-{[2-(difluoromethyl)-4-(pyrimidin-2-yl)phenyl]amino}hexyl)-2-(hydroxymethyl)piperidine-3,4,5-triol

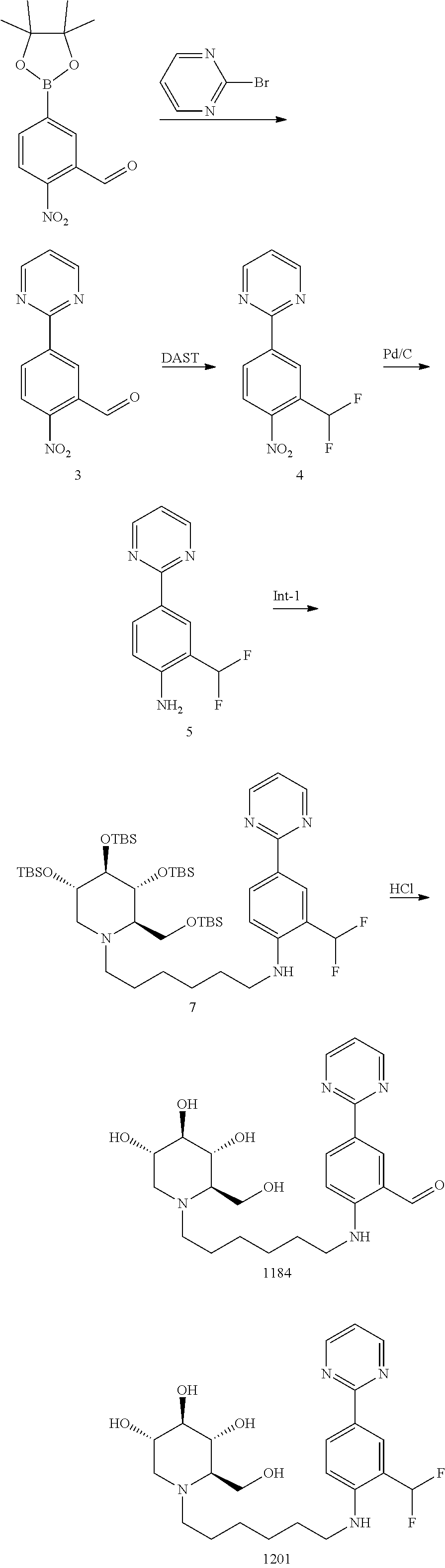

2-Nitrobenzaldehyde-5-boronate (1.5 g), toluene:EtOH:water (60 mL), Na₂CO₃ (3 eq), 2-bromo-pyrimidine (1 eq), degassed 15 minutes N₂, added Pd(dppf)Cl₂ (0.1 eq), reacted at 80° C. 8 hours. Reaction was cooled to room temperature, volatiles evaporated, residue dissolved in EtOAc (50 mL) and washed with water (20 mL). The organic layer was dried over Na₂SO₄, filtered, concentrated, purified on silica [5% EtOAc in hexane] to afford 2.1 g of 3.

To 3 (2.1 g) in DCM (80 mL) at 0° C. added diethylaminosulfur trifluoride (1.2 eq) then reacted at room temperature for 4 hours quenched with saturated NaHCO₃ and extracted with DCM. The organic layer was dried over anhydrous Na₂SO₄, concentrated, purified by column [10% EtOAc in hexane] to afford 1.9 g of 4 as off white solid.

4 (1.9 g), MeOH (30 mL), 10% Pd/C (50% wet, 300 mg), kept under mild H₂ pressure (balloon) at room temperature for 5 hours. The reaction mass was filtered through pad of CELITE and the filtrate concentrated to afford 1.4 g of 5 as off-white thick syrup.

5 (800 mg), Int-1 (Example 1, 1.0 eq), MeOH (60 mL) mixed at 0° C., AcOH (0.1 mL), NaCNBH₃ (1.5 eq) added then room temperature for 16 hours. Reaction mass was diluted with water, extracted with EtOAc, the organic layer dried over anhydrous Na₂SO₄ and concentrated, residue was purified on silica [10% EtOAc in hexane] to afford 500 mg of 7.

To 7 (400 mg) in MeOH (10 mL) at 0° C., added 4.0 M HCl in 1,4-dioxane (4 mL) and reacted at room temperature 2 hours. The solvent was removed, residue after preparative HPLC purification yielded 4 mg of 1201 and 6 mg of 1184.

### Example 75: Synthesis of Compounds 1207 ((2R,3R,4R,5S)-2-(hydroxymethyl)-1-(6-{[2-methoxy-4-(1,3-oxazol-2-yl)phenyl]amino}hexyl)piperidine-3,4,5-triol) and 1199 ((2R,3R,4R,5S)-2-(hydroxymethyl)-1-(6-{[2-methoxy-4-(pyrazin-2-yl)phenyl]amino}hexyl)piperidine-3,4,5-triol)

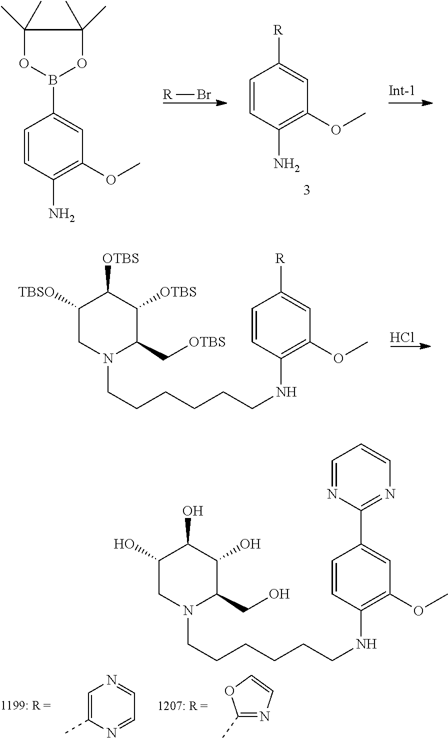

Preparation of 1207 (2R,3R,4R,5S)-2-(hydroxymethyl)-1-(6-{[2-methoxy-4-(1,3-oxazol-2-yl)phenyl]amino}hexyl)piperidine-3,4,5-triol: Mixed 4-amino-3-methoxy-phenyl boronate (800 mg), Na₂CO₃ (3 eq), toluene:EtOH:water (18 mL), 2-bromooxazole (1 eq), degassed 15 minutes N₂, added Pd(dppf)Cl₂ (0.1 eq), reacted 4 hours at 80° C. Cooled to room temperature and volatiles evaporated, residue was dissolved in EtOAc (50 mL), washed with water (20 mL), organic layer was dried over Na₂SO₄, filtered, concentrated, and purified on silica [5% EtOAc in hexane] to afford 400 mg of 3.

Mixed 3 (350 mg), Int-1 (Example 1, 1.5 eq), MeOH (20 mL) at 0° C., added AcOH (0.1 mL), NaCNBH₃ (1.5 eq), reacted 16 hours at room temperature. Reaction mass diluted with water and extracted with EtOAc. The organic layer was dried over anhydrous Na₂SO₄, concentrated, and purified on silica [10% EtOAc in hexane] to afford 800 mg of 5.

Mixed 5 (400 mg), MeOH (10 mL) at 0° C., added 4.0 M HCl in 1,4-dioxane (2 mL), reacted 2 hours at room temperature. The reaction mass was concentrated then preparative HPLC purification yielded 30 mg of 1207.

Preparation of 1199 (2R,3R,4R,5S)-2-(hydroxymethyl)-1-(6-{[2-methoxy-4-(pyrazin-2-yl)phenyl]amino}hexyl)piperidine-3,4,5-triol: 4-amino-3-methoxyphenyl-boronate (500 mg), 2-bromopyrazine (1.0 eq), degassed 15 minutes N₂, added Pd(dppf)Cl₂ (0.1 eq), Na₂CO₃ (3 eq), toluene (5 mL), EtOH (5 mL) and H₂O (5 mL), reacted 6 hours at 90° C. The reaction was diluted with water and extracted with EtOAc. The organic layer was dried over anhydrous Na₂SO₄, concentrated, and purified on silica [30% EtOAc:hexane] to afford 220 mg of 3 as thick syrup.

3 (250 mg), MeOH (5 mL), Int-1 (1.2 eq), AcOH (cat.), NaCNBH₃ (1.5 eq), reacted at room temperature for 16 hours. The reaction was diluted with water and extracted with EtOAc. The organic layer was dried over anhydrous Na₂SO₄, concentrated, and purified on silica [20% EtOAc:hexane] to afford 240 mg of 6 as thick syrup.

6 (240 mg), MeOH (5 mL), 4.0 N HCl in 1,4-dioxane (2.4 mL) reacted 16 hours at room temperature. Volatiles were removed. Preparative HPLC purification afforded 45 mg of 1199 as thick yellow syrup.

### Example 76: Synthesis of Compounds 1196 ((2R,3R,4R,5S)-1-(6-{[3-chloro-5-(pyrazin-2-yl)phenyl]amino}hexyl)-2-(hydroxymethyl)piperidine-3,4,5-triol), 1211 ((2R,3R,4R,5S)-1-(6-{[3-chloro-5-(1,3-oxazol-2-yl)phenyl] amino}hexyl)-2-(hydroxymethyl)piperidine-3,4,5-triol), and 1212 ((2R,3R,4R,5S)-1-(6-{[3-chloro-5-(pyrimidin-2-yl)phenyl] amino}hexyl)-2-(hydroxymethyl)piperidine-3,4,5-triol)

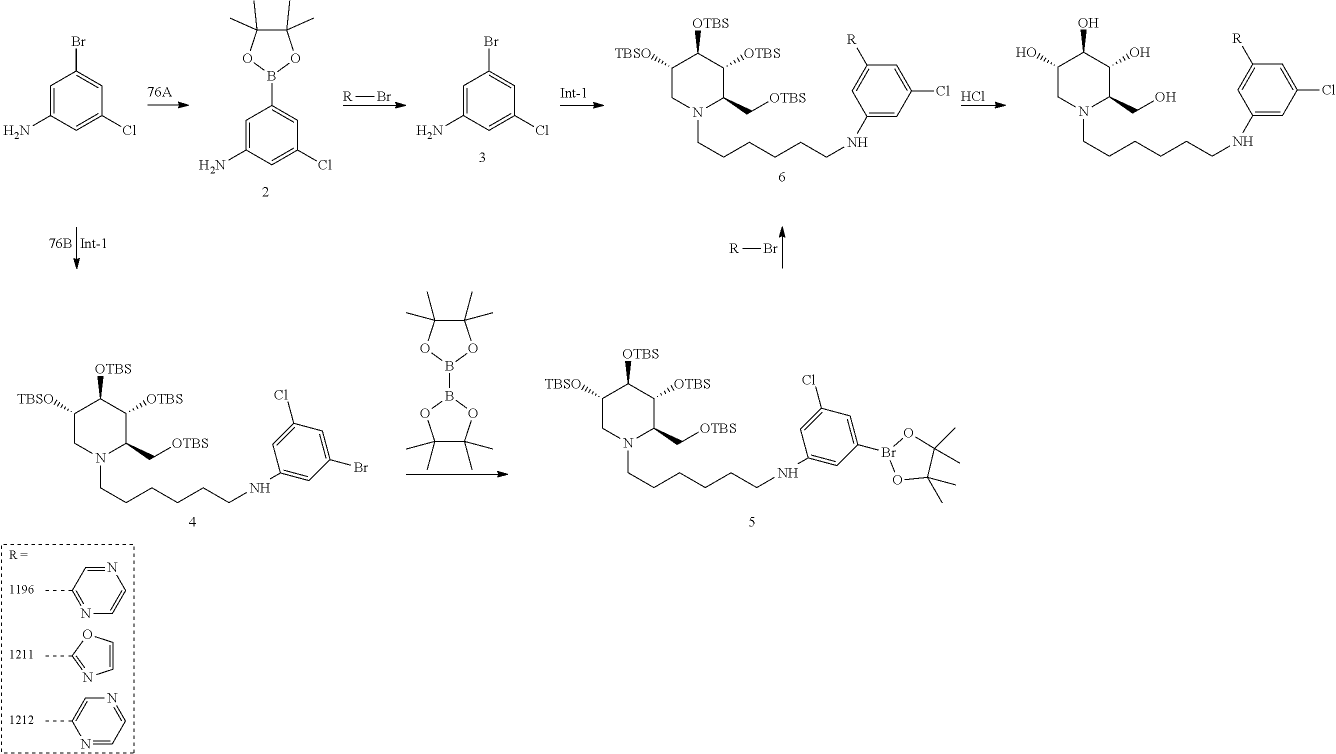

Preparation of 1211 (76A) (2R,3R,4R,5S)-1-(6-{[3-chloro-5-(1,3-oxazol-2-yl)phenyl]amino}hexyl)-2-(hydroxymethyl)piperidine-3,4,5-triol: 3-Bromo-5-chloroaniline (1 g), bis(pinacolato) diboron (1.5 eq), 1,4-dioxane (20 mL), KOAc (3 eq), degassed 15 minutes N₂, added Pd(dppf)Cl₂ (0.1 eq) reacted at 90° C. for 2 hours. The reaction was cooled to room temperature and volatiles evaporated. The residue was dissolved in water (10 mL) and extracted with EtOAc (2×10 mL), dried over Na₂SO₄, filtered, concentrated and purified on silica [20% EtOAc in hexane] to afford 850 mg of 2.

Reacted 2 (900 mg), toluene:EtOH:water (40 mL), 2-bromooxazole (1.0 eq.), LiOH (3 eq), degassed 15 minutes N₂, added Pd(dppf)Cl₂ (0.1 eq) at 80° C. for 4 hours. Cooled to room temperature and water was added and extracted with EtOAc (2×50 mL). The organic layer was dried over Na₂SO₄, filtered, concentrated, and purified on silica [20%-40% EtOAc in hexane] to afford 410 mg of 3.

Mixed Int-1 (1.5 g), MeOH (30 mL), 3 (1 eq), AcOH (cat.) at 0° C., added NaCNBH₃ (1.5 eq), reacted at room temperature 16 hours. Concentrated the reaction, dissolved residue in water (100 mL), extracted with EtOAc (2×100 mL). The organic layer was dried over Na₂SO₄, filtered, concentrated, and purified on silica [10-20% EtOAc in hexane] to afford 500 mg of 6.

6 (500 mg), 1,4-dioxane (20 mL), 4.0 N HCl in dioxane (5 mL) reacted at room temperature 16 hours. Solvent was removed and residue purified by preparative HPLC to obtain 45 mg of 1211.

Preparation of 1196 (76A) (2R,3R,4R,5S)-1-(6-{[3-chloro-5-(pyrazin-2-yl)phenyl]amino}hexyl)-2-(hydroxymethyl)piperidine-3,4,5-triol: Reacted 2 (150 mg), 2-bromo-pyrazine (1.5 eq), Pd(dppf)Cl₂ (0.1 eq), Na₂CO₃ (3 eq), toluene:EtOH:H₂O (1:1:1, 9 mL) 4 hours at 80° C. after degassing 15 minutes with N₂. Quenched reaction with water and diluted with EtOAc (25 mL). The organic layer was separated, washed with brine, dried over Na₂SO₄, concentrated, and purified on silica [40% EtOAc:hexane] to afford 150 mg of 3-chloro-5-(pyrazin-2-yl)aniline as colorless thick syrup.

Mixed 3 (150 mg), MeOH (8 mL), Int-1 (Example 1, 1 eq), AcOH (cat.) at 0° C., added NaCNBH₃ (1.5 eq), reacted 16 hours at room temperature. The reaction was concentrated, residue was dissolved in water (20 mL) and extracted with EtOAc (2×10 mL). The organic layer was dried over Na₂SO₄, filtered, concentrated, and purified on silica [10% EtOAc in hexane] to afford 300 mg of 7.

7 (300 mg), MeOH (10 mL) mixed at 0° C., added 4.0 M HCl in 1,4-dioxane (3 mL), reacted 4 hours at room temperature. The reaction mass was concentrated and residue purified by preparative HPLC to afford 62 mg of 1196 as off-white solid.

Preparation of 1212 (76B) (2R,3R,4R,5S)-1-(6-{[3-chloro-5-(pyrimidin-2-yl)phenyl]amino}hexyl)-2-(hydroxymethyl)piperidine-3,4,5-triol: Int-1 (2 g), MeOH (40 mL), 3-bromo-5-chloroaniline (1 eq), AcOH (1 mL), NaCNBH₃ (1.5 eq) reacted at room temperature for 16 hours. The volatiles were evaporated, the residue dissolved in EtOAc and washed with saturated NaHCO₃ and brine, the organic was dried over Na₂SO₄, concentrated, and purified on silica [10-20% EtOAc-hexane] to obtain 1.2 g of 4.

4 (1.2 g), bis(pinacolato) diboron (1.5 eq), Pd(dppf)Cl₂ (0.1 eq), KOAc (3 eq), DMSO (12 mL) reacted at 90° C. 16 hours after degassing 15 minutes with N₂. Reaction mass was diluted with water and extracted with EtOAc, the organic layer was washed with brine then dried over Na₂SO₄ and concentrated to afford 890 mg of 5.

2-Bromopyrimidine (250 mg), 5 (0.5 eq), Pd(dppf)Cl₂ (0.1 eq), LiOH (3 eq), toluene:EtOH:water (2:1:1 20 mL), degassed 15 minutes N₂, reacted in microwave at 110° C. for 90 minutes. Added water and extracted with EtOAc. The organic layer was washed with brine, dried over Na₂SO₄ and concentrated to afford 600 mg of 6.

6 (600 mg), 1,4-dioxane (6 mL), 4.0 N HCl in 1,4-dioxane (6 mL) reacted at room temperature for 16 hours. Volatiles were evaporated and preparative HPLC purification yielded 51 mg of 1212.

### Example 77: Synthesis of Compound 1200 (2R,3R,4R,5S)-1-[2-(4-{2-[(4-azido-2-nitrophenyl)amino]ethyl}phenyl)ethyl]-2-(hydroxymethyl)piperidine-3,4,5-triol)

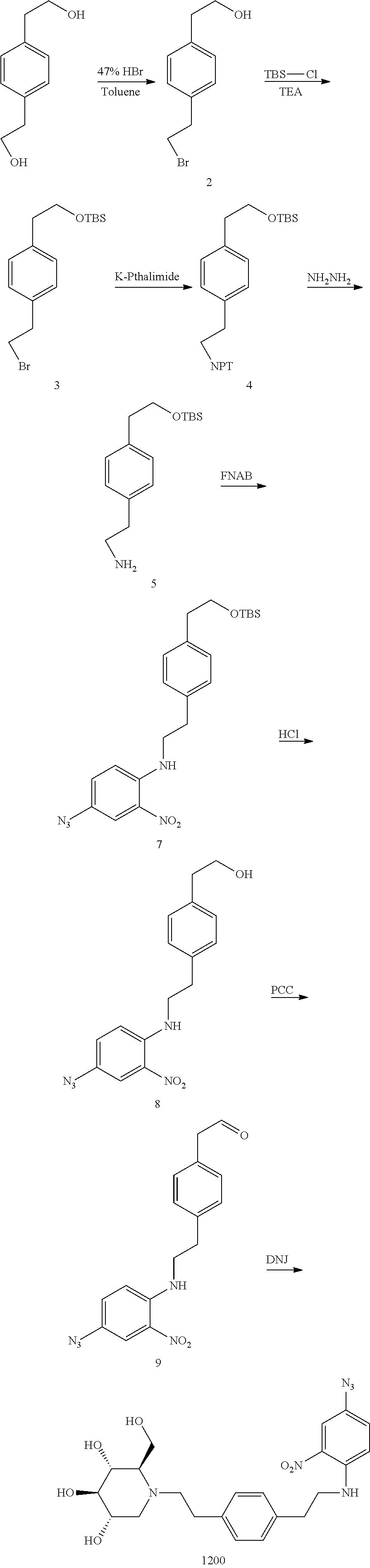

Mixed 1,4-bis(2-hydroxyethyl)benzene (5.0 g), toluene (75 mL), 47% aqueous HBr (25 mL) 2 hours at 80° C. The reaction was diluted with water and extracted with EtOAc. The organic layer was dried over anhydrous Na₂SO₄, concentrated, and purified on silica [30% EtOAc:hexane] to afford 5.5 g of 2 as thick syrup.

Mixed 2 (5.5 g), DCM (60 mL) at 0° C., added TEA (3.0 eq), TBDMS-Cl (1.2 eq), DMAP (cat), reacted 5 hours at room temperature. Diluted with water and extracted with EtOAc. The organic layer was dried over anhydrous Na₂SO₄ and concentrated to afford 8.1 g of 3 as thick syrup.

Mixed 3 (8.1 g), DMF (15 mL), potassium phthalamide (1.3 eq) at 60° C. for 16 hours. Diluted with water and extracted with EtOAc. The organic layer was dried over anhydrous Na₂SO₄, concentrated, and purified on silica [40% EtOAc:hexane] to afford 3.5 g of 4.

4 (3.5 g), EtOH (30 mL), 35% aqueous NH₂NH₂ (5.0 eq) reacted at room temperature 16 hours. The reaction was diluted with water and extracted with EtOAc. The organic layer was washed with water (2×100 mL), dried over anhydrous Na₂SO₄, and concentrated to afford 2.0 g of 5.

5 (2.0 g), FNAB (1.1 eq), 1,4-dioxane (30 mL), TEA (3.0 eq) reacted at 80° C. for 16 hours. The reaction was diluted with water and extracted with EtOAc. The organic layer was washed with water (2×100 mL), dried over anhydrous Na₂SO₄, concentrated, and purified on silica [5% EtOAc in hexane] to afford 1.2 g of 7.

7 (1.2 g), THE (20 mL) mixed at 0° C., added TBAF (1.0 Min THF, 1.5 eq) and reacted at room temperature for 3 hours. The reaction was diluted with saturated NaHCO₃ and extracted with EtOAc. The organic layer was dried over anhydrous Na₂SO₄, concentrated, and purified on silica [30% EtOAc:hexane] to afford 500 mg of 8 as thick syrup.

8 (500 mg), DCM (2 mL) mixed at 0° C., added DMP (1.5 eq), reacted 4 hours at room temperature. The reaction was diluted with saturated NaHCO₃, extracted with DCM and washed with water (2×100 mL). The organic layer was dried over anhydrous Na₂SO₄, concentrated, and purified on silica [30% EtOAc in hexane] to afford 400 mg of 9.

9 (400 mg), MeOH (30 mL), DNJ (0.8 eq), NaCNBH₃ (3 eq), AcOH (0.1 mL) mixed at 0° C. then maintained at room temperature. After 16 hours the solvent was removed and preparative HPLC purification afforded 150 mg of 1200.

### Example 78: Synthesis of Compounds 1198 ((2R,3R,4R,5S)-1-(6-{[2-chloro-4-(pyrazin-2-yl)phenyl]amino}hexyl)-2-(hydroxymethyl)piperidine-3,4,5-triol), 1202 ((2R,3R,4R,5S)-1-(6-{[2-chloro-4-(pyridazin-3-yl)phenyl]amino}hexyl)-2-(hydroxymethyl)piperidine-3,4,5-triol), and 1203 ((2R,3R,4R,5S)-1-(6-{[2-chloro-4-(1,3-oxazol-2-yl)phenyl]amino}hexyl)-2-(hydroxymethyl)piperidine-3,4,5-triol)

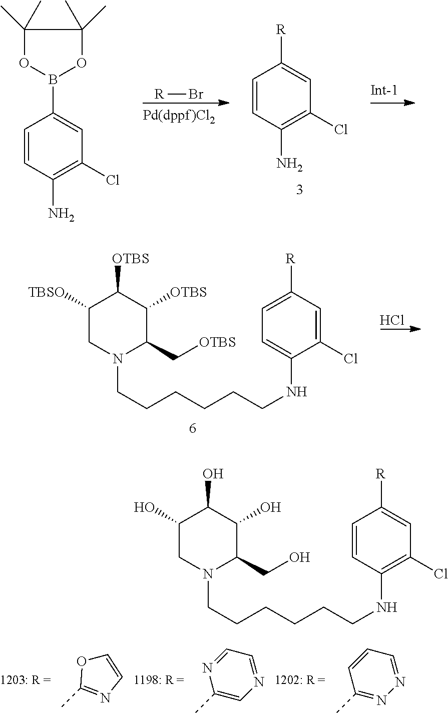

Preparation of 1198 (2R,3R,4R,5S)-1-(6-{[2-chloro-4-(pyrazin-2-yl)phenyl]amino}hexyl)-2-(hydroxymethyl)piperidine-3,4,5-triol: Reacted 4-amino-3-chlorobenzene boronate (1, 500 mg), 2-bromopyrazine (1.0 eq), Pd(dppf)Cl₂ (0.1 eq), Na₂CO₃ (3 eq), toluene:EtOH:water (1:1:1, 15 mL) 90° C. for 6 hours after degassing 15 minutes with N₂. The reaction was diluted with water and extracted with EtOAc. The organic layer was dried over anhydrous Na₂SO₄, concentrated, and purified on silica [30% EtOAc:hexane] to afford 210 mg of 3 as thick syrup.

3 (240 mg), MeOH (5 mL), Int-1 (1.2 eq), AcOH (cat.), NaCNBH₃ (1.5 eq) reacted at room temperature for 16 hours. The reaction was diluted with water and extracted with EtOAc. The organic layer was dried over anhydrous Na₂SO₄, concentrated, and purified on silica [20% EtOAc:hexane] to afford 220 mg of 6 as thick syrup.

6 (220 mg), MeOH (5 mL), 4.0 N HCl in 1,4-dioxane (2.4 mL) mixed at 0° C. then increased to room temperature and maintained 16 hours. Volatiles were concentrated. Preparative HPLC purification afforded 41 mg of 1198 as off-white solid.

Preparation of 1202 (2R,3R,4R,5S)-1-(6-{[2-chloro-4-(pyridazin-3-yl)phenyl]amino}hexyl)-2-(hydroxymethyl)piperidine-3,4,5-triol: 1 (1.0 g), 3-bromopyridazine (1.5 eq), Pd(dppf)Cl₂ (0.1 eq), LiOH (3 eq), toluene:EtOH:water (1:1:1, 45 mL) reacted at 90° C. for 24 hours after degassed 15 minutes N₂. Water was added reaction and mix extracted with EtOAc. The organic layer was dried over Na₂SO₄, concentrated, and purified on silica [20-40% EtOAc:hexane] to provide 120 mg of 3.

Mixed Int-1 (100 mg, Example 1), MeOH (5 mL), 3 (1.5 eq), AcOH (cat.), NaCNBH₃ (1.5 eq) 24 hours at room temperature. Volatiles were evaporated, residue dissolved in EtOAc and washed with water and brine, organic layer was dried over Na₂SO₄ and concentrated to obtain 6.

6 (300 mg), 1,4-dioxane (6 mL), 4.0 N HCl in 1,4-dioxane (6 mL) reacted 16 hours at room temperature. Preparative HPLC purification yielded 130 mg of material which after normal phase preparative HPLC purification yielded 42 mg of 1202.

Preparation of 1203 (2R,3R,4R,5S)-1-(6-{[2-chloro-4-(1,3-oxazol-2-yl)phenyl]amino}hexyl)-2-(hydroxymethyl)piperidine-3,4,5-triol: 1 (200 mg), 2-bromooxazole (1.5 eq), Pd(dppf)Cl₂ (0.1 eq), Na₂CO₃ (3 eq), toluene:EtOH:water (1:1:1, 9 mL), degassed 15 minutes N₂, reacted at 90° C. 3 hours. Added ice-cold water and extracted with EtOAc (2×15 mL), dried over Na₂SO₄, filtered, concentrated, and purified on silica [20-30% EtOAc-hexane]yielded 70 mg of 3.

Int-1 (500 mg), 3 (0.8 eq), MeOH (10 mL), AcOH (cat.), NaCNBH₃ (1.5 eq) reacted at room temperature 16 hours. The volatiles were evaporated and 20 mL water was added. The aqueous layer was extracted with EtOAc (2×20 mL). The combined organic layer was dried over Na₂SO₄ and concentrated to afford 6.

6 (400 mg), MeOH (4 mL), 4.0 N HCl in 1,4-dioxane (8 mL) reacted 16 hours at room temperature. The solvent was evaporated, and preparative HPLC purification and lyophilization provided 40 mg of 1203.

### Example 79: Synthesis of Compounds 1221 ((2R,3R,4R,5S)-1-[3-({3-[(4-azido-2-nitrophenyl)amino]propyl}(5-methoxypentyl)amino)propyl]-2-(hydroxymethyl)piperidine-3,4,5-triol) and 1234 (4-({3-[(4-azido-2-nitrophenyl)amino]propyl}({3-[(2R,3R,4R,5S)-3,4,5-trihydroxy-2-(hydroxymethyl)piperidin-1-yl]propyl})amino)butanamide)

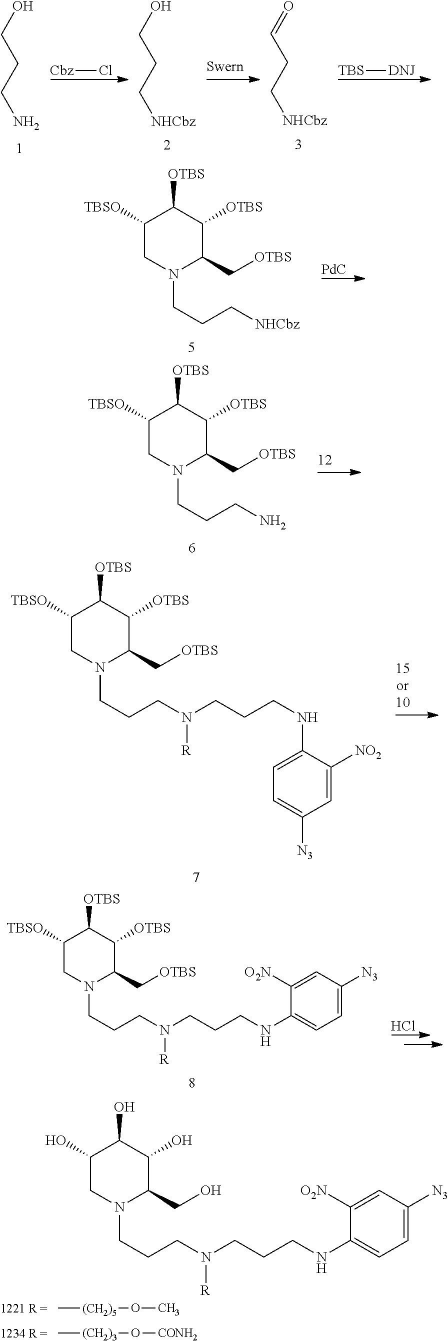

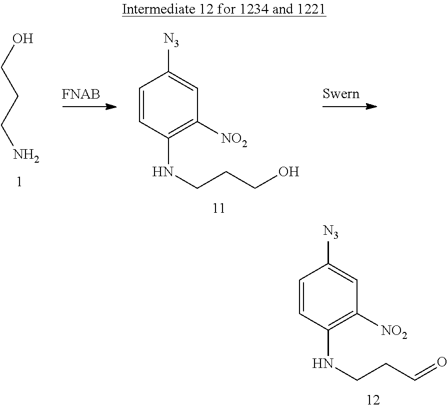 [SMILES: NCCCO ; [N-]=[N+]=Nc1ccc(NCCCO)c([N+](=O)[O-])c1 ; [N-]=[N+]=Nc1ccc(NCCC=O)c([N+](=O)[O-])c1]

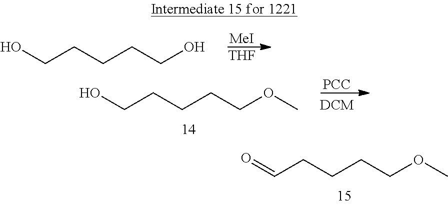

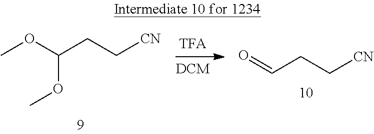

Preparation of 7: 3-aminopropan-1-ol (1, 10 g), DCM (100 mL), saturated NaHCO₃ (100 mL), Cbz-Cl (1.0 eq. 50% in toluene), mixed at 0° C., raised to room temperature for 16 hours. The reaction was quenched with ice-cold water (150 mL) and extracted with DCM (2×150 mL). The combined organic layer was washed with 150 mL water, dried over Na₂SO₄, filtered, and concentrated, to afford 15 g of 2 as white solid.

To (COCl)₂ (2 eq) in DCM (120 mL) at −78° C. added DMSO (4 eq) and 2 (6 g), reacted 2 hours added Et₃N (5 eq), reacted 2 hours. The reaction mixture was diluted with water and DCM (50 mL). The organic layer was separated, washed with brine, dried over Na₂SO₄, concentrated to afford 6 g of 3.

3 (6 g), MeOH (20 mL), TBS-DNJ (0.8 eq), AcOH (cat.), NaCNBH₃ (1.5 eq) reacted room temperature for 16 hours. The reaction was quenched with ice-cold water (50 mL) and extracted with EtOAc (2×60 mL). The combined organic layer was dried over Na₂SO₄, concentrated, and purified on silica [5% EtOAc:hexane] to afford 10 g of 5 as colorless thick syrup.

5 (10 g), MeOH (100 mL), 10% Pd/C (8 g, 50% wet) at room temperature reacted under H₂ (balloon pressure) for 24 hours. Reaction mass was filtered through CELITE bed and washed with MeOH (100 mL). The filtrate was concentrated to afforded 8 g of 6 as colorless thick syrup.

Mixed FNAB (3 g), 1,4-dioxane (60 mL), TEA (3.0 eq), 1 (1.5 eq) at room temperature, increased to 80° C. for 16 hours. The reaction was concentrated, quenched with ice-cold water (25 mL) and extracted with EtOAc (2×35 mL). Organic layer was washed with water (30 mL), dried over Na₂SO₄, filtered, and concentrated to afford 3.5 g of 11 as orange-red solid.

Oxalyl chloride (2 eq), DCM (30 mL), −78° C., DMSO (4 eq), 11 (2 g) reacted 2 hours added Et₃N (5 eq), reacted 2 hours. The reaction was quenched with water and diluted with DCM. The organic layer was separated, washed with brine and dried over Na₂SO₄. The organic layer was concentrated to afford 2 g of 12.

12 (2 g), MeOH (50 mL), 6 (1 eq), AcOH (cat.), NaCNBH₃ (1.5 eq), reacted 16 hours at room temperature. The reaction was quenched with ice-cold water (50 mL) and extracted with DCM (2×50 mL). The combined organic layers were dried over Na₂SO₄ filtered, concentrated, and purified by silica [80% EtOAc:hexane] to afford 2 g of 7 as colorless thick syrup.

Preparation of 1221 (2R,3R,4R,5S)-1-[3-({3-[(4-azido-2-nitrophenyl)amino]propyl}(5-methoxypentyl)amino)propyl]-2-(hydroxymethyl)piperidine-3,4,5-triol: 13 (10 g), THE (200 mL), tBuOK (1.0 eq), mixed at room temperature for 1 hour added Mel (1.0 eq), reacted at room temperature for 16 hours. The reaction was quenched with ice-cold water (100 mL) and extracted with DCM (2×50 mL). The combined organic layers were dried over Na₂SO₄ filtered, concentrated, and purified by silica [50% EtOAc:hexane] to afforded 3 g of 14 as colorless thick syrup.

14 (1 g), CHCl₃ (20 mL), PCC (2.5 eq) reacted 4 hours at room temperature. The reaction mass was filtered through a CELITE bed and washed with DCM (20 mL). The filtrate was concentrated to afforded 700 mg of 15 as colorless thick syrup.

Mixed 15 (700 mg), MeOH (15 mL), 7 (1 eq), AcOH (cat.), NaCNBH₃ (1.5 eq) at room temperature for 16 hours. Quenched with ice-cold water (50 mL) and extracted with DCM (2×50 mL). The combined organic layers were dried over Na₂SO₄ filtered, concentrated, and purified on silica [20% EtOAc:hexane] to afford 200 mg of 8 as orange red thick syrup.

Mixed 8 (200 mg), MeOH (5 mL), 4M HCl in 1,4-dioxane (2 mL) at 0° C. then at room temperature for 1 hour. The reaction was concentrated and purified by preparative HPLC. Two batches were blended using MeOH and dried to afford 37 mg of 1221 as orange-red thick syrup.

Preparation of 1234 4-({3-[(4-azido-2-nitrophenyl)amino]propyl}({3-[(2R,3R,4R,5S)-3,4,5-trihydroxy-2-(hydroxymethyl)piperidin-1-yl]propyl})amino)butanamide: 9 (4,4-dimethoxybutanenitrile, 3 g), DCM (60 mL), TFA:H₂O (1:1, 15 mL) mixed at 0° C. then 4 hours at room temperature. The reaction was quenched with ice-cold water (50 mL), extracted with DCM (2×50 mL), the combined organic layer was dried over Na₂SO₄ and concentrated to afford 1.0 g of 10 as oily liquid.

Mixed 7 (250 mg), MeOH (5 mL), 10 (1 eq), AcOH (cat.), NaCNBH₃ (1.5 eq) 16 hours at room temperature. Quenched with ice-cold water (20 mL) and extracted with DCM (2×10 mL). The combined organic layer was dried over Na₂SO₄, filtered, concentrated, and purified on silica [80% EtOAc:hexane] to afford 280 mg of 8 as orange-red thick syrup.

8 was converted to the target molecule in two steps. Firstly, 8 (280 mg), MeOH:DMSO (10 mL, 1:1), 1 N NaOH (15 eq), H₂O₂ (15 eq) were mixed at 0° C. then increased to room temperature for 16 hours. The reaction was quenched with ice-cold water (20 mL) and extracted with EtOAc (2×15 mL). The combined organic layer was dried over Na₂SO₄, filtered, concentrated, and purified on silica [100% EtOAc] to afford 180 mg of the amide (structure not shown) as orange red thick syrup. The amide (160 mg) was then de-protected by mixing with MeOH (5 mL) and 4 M HCl in 1,4-dioxane (1.6 mL) at 0° C. and then at room temperature for 1 hour. Reaction mass was concentrated and purified by preparative HPLC to afford 21 mg of 1234.

### Example 80: Synthesis of Compounds 1235 (3-({2-[(4-azido-2-nitrophenyl)amino]ethyl}({2-[(2S,3S,4S,5R)-3,4,5-trihydroxy-2-(hydroxymethyl)piperidin-1-yl]ethyl})amino)propenamide) and 1226 (3-[(2-{[2-nitro-4-(pyrimidin-2-yl)phenyl]amino}ethyl)({2-[(2R,3R,4R,5S)-3,4,5-trihydroxy-2-(hydroxymethyl)piperidin-1-yl]ethyl})amino]propenamide)

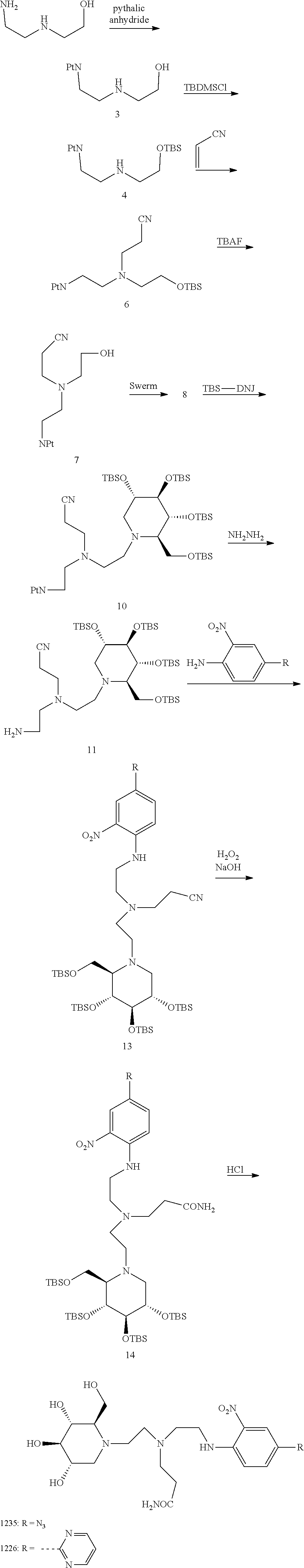

Preparation of 11: 2-((2-aminoethyl)amino)ethan-1-ol (25 g), toluene (800 mL), phthalic anhydride (1.0 eq), mixed at room temperature then at 120° C. for 3 hours. Volatiles were concentrated to afford 45 g of 3 as yellow solid. Then immediately 3 (45 g), DMF (450 mL), TEA (5.0 eq), TBDMS-Cl (1.2 eq) mixed at 0° C. then reacted at room temperature 16 hours. Reaction was quenched with ice-cold water (500 mL), extracted with EtOAc (2×300 mL), organic layer was dried over Na₂SO₄, concentrated, and purified on silica [50% EtOAc-hexane]giving 4 as colorless syrup.

4 (15 g), MeOH (300 mL), acrylonitrile (15 mL) reacted 24 hours at room temperature. The volatiles were concentrated and major non-polar spot was isolated on silica [20% EtOAc:Hexane] to afforded 8 g of 6 as colorless thick syrup.

6 (8 g), THE (160 mL), TBAF (1.2 eq, 1.0 M in THF) mixed at 0° C. then at room temperature 16 hours. The reaction was quenched with ice-cold water (100 mL), extracted with EtOAc (2×100 mL), organic layer was dried over Na₂SO₄, concentrated, and purified on silica [50% EtOAc-hexane] to afford 4 g of 7 as colorless thick syrup.

Mixed 7 (3 g), (COCl)₂ (2 eq), DCM (30 mL), DMSO (4 eq) at −78° C. 2 hours added Et₃N (5 eq), mixed 1 hour. Quenched with water, diluted with DCM, washed with brine, the organic layer was dried over Na₂SO₄, concentrated to afford 2.5 g of aldehyde 8 (not shown) which was moved immediately to next step.

8 (2.5 g), TBS-DNJ (0.8 eq), MeOH (50 mL) mixed at 0° C. added NaCNBH₃ (1.5 eq), AcOH (cat.) mixed at room temperature 16 hours. The reaction was quenched with ice-cold water (40 mL), extracted with EtOAc (2×50 mL), combined organic layer was dried over Na₂SO₄, concentrated, and purified by column [20% EtOAc-hexane] to afford 3 g of 10 as colorless thick syrup.

10 (3 g), EtOH (60 mL), NH₂NH₂·H₂O (2 eq) reacted at room temperature 16 hours. Reaction was quenched with ice-cold water (10 mL), extracted with EtOAc (2×10 mL), combined organic layer was dried over Na₂SO₄ and concentrated to afford 2.0 g of 11 as colorless thick syrup.

Preparation of 1235 3-({2-[(4-azido-2-nitrophenyl)amino]ethyl}({2-[(2S,3S,4S,5R)-3,4,5-trihydroxy-2-(hydroxymethyl)piperidin-1-yl]ethyl})amino)propanamide: 11 (1 g), 1,4-dioxane (20 mL), FNAB (1.0 eq), TEA (3 eq) mixed at room temperature then 80° C. 16 hours. Reaction was quenched with ice-cold water (30 mL), extracted with EtOAc (2×20 mL), organic layers were dried over Na₂SO₄, concentrated, and purified by column [10% EtOAc-hexane] to afford 1 g of 13 as orange red thick syrup.

13 (300 mg), DMSO:MeOH (1:1, 10 mL), H₂O₂ solution (15 eq), 1M NaOH (15 eq) mixed at 0° C. then room temperature, 16 hours. Reaction was quenched with ice-cold water (10 mL), extracted with EtOAc (2×20 mL), combined organic layer was dried over Na₂SO₄, concentrated, and purified by column [40% EtOAc-hexane] to afford 200 mg of 14 as orange red thick syrup.

14 (180 mg), MeOH (5 mL), 4M HCl in 1,4-dioxane (2 mL) mixed at 0° C. then room temperature for 2 hours. Volatiles were concentrated and preparative HPLC purification afforded 43 mg 1235 as an orange-red thick syrup.

Preparation of 1226 3-[(2-{[2-nitro-4-(pyrimidin-2-yl)phenyl]amino}ethyl)({2-[(2R,3R,4R,5S)-3,4,5-trihydroxy-2-(hydroxymethyl)piperidin-1-yl]ethyl})amino]propanamide: Mixed 11 (1 g), 1,4-dioxane (20 mL), TEA (3 eq), 2-(4-fluoro-3-nitrophenyl)pyrimidine (1.0 eq) at room temperature, heated at 80° C. 16 hours. The reaction was quenched with ice-cold water (30 mL) and extracted with EtOAc (2×20 mL). The combined organic layer was dried over Na₂SO₄, concentrated, and purified on silica [10% EtOAc-hexane] to afford 1 g of 13 as yellow thick syrup.

13 (300 mg), DMSO:MeOH (1:1, 10 mL), H₂O₂ (15 eq), 1 M NaOH (15 eq) mixed at 0° C. then at room temperature 16 hours. The reaction was quenched with ice-cold water (10 mL) and extracted with EtOAc (2×20 mL). The combined organic layer was dried over Na₂SO₄, concentrated, and purified by column [40% EtOAc:hexane] to afford 200 mg of 14 as orange red thick syrup.

14 (180 mg), MeOH (6 mL), 4M HCl in 1,4-dioxane (1.8 mL) mixed at 0° C. then at room temperature for 2 hours. The volatiles were concentrated and after preparative HPLC purification afforded 50 mg of 1226 as yellow thick syrup.

### Example 81: Synthesis of Compounds 1233 ((2R,3R,4R,5S)-2-(hydroxymethyl)-1-(6-{[2-methyl-4-(pyrimidin-2-yl)phenyl]amino}hexyl)piperidine-3,4,5-triol), 1231 ((2R,3R,4R,5S)-2-(hydroxymethyl)-1-(6-{[2-methoxy-4-(pyrimidin-2-yl)phenyl]amino}hexyl)piperidine-3,4,5-triol), 1229 ((2R,3R,4R,5S)-1-(6-{[2-fluoro-4-(pyrimidin-2-yl)phenyl]amino}hexyl)-2-(hydroxymethyl)piperidine-3,4,5-triol), and 1228 ((2R,3R,4R,5S)-1-(6-{[2-chloro-4-(pyrimidin-2-yl)phenyl]amino}hexyl)-2-(hydroxymethyl)piperidine-3,4,5-triol)

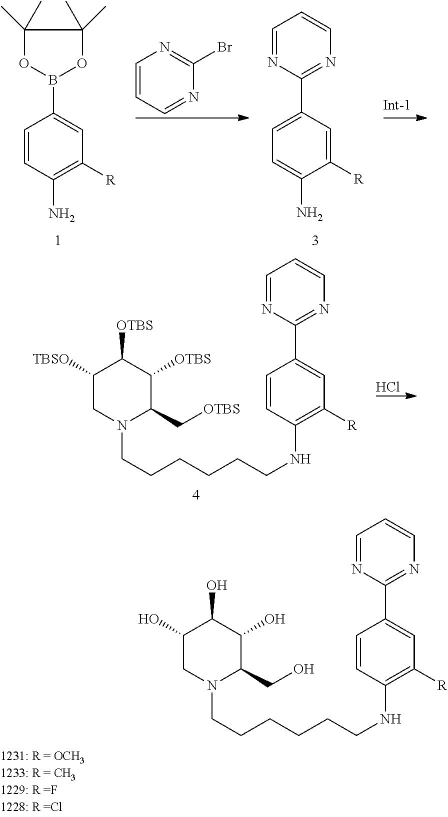

Preparation of 1233 (2R,3R,4R,5S)-2-(hydroxymethyl)-1-(6-{[2-methyl-4-(pyrimidin-2-yl)phenyl]amino}hexyl)piperidine-3,4,5-triol: Purged 4-amino-3-methylphenylboronate (500 mg), 2-bromopyrimidine (0.8 eq), Na₂CO₃ (3 eq), toluene:EtOH:H₂O (15 mL) with Ar for 30 minutes, added Pd(dppf)Cl₂·DCM (0.1 eq) and reacted at 80° C. for 2 hours. The reaction was diluted with water and extracted with EtOAc. The organic layer was dried over Na₂SO₄, filtered, evaporated, and purified on silica [20-30% EtOAc:hexane] to give 230 mg of 3.

3 (230 mg), Int-1 prepared as Example 1 (1.5 eq), MeOH (10 mL) mixed at 0° C., added NaCNBH₃ (1.5 eq), AcOH (cat), reacted at room temperature 16 hours. The contents were evaporated and the residue was diluted with DCM and washed with water and brine. The organic layer was separated, dried over Na₂SO₄, concentrated, and purified on COMBIFLASH [100-200 silica gel, eluting with 5-10% EtOAc:hexane] to give 300 mg of 4.

Mixed 4 (300 mg), 4N HCl in 1,4-dioxane (3 mL), 1,4-dioxane (9 mL) at room temperature for 6 hours. Volatiles were evaporated then preparative HPLC purification gave 30 mg of 1233.

Preparation of 1228 (2R,3R,4R,5S)-1-(6-{[2-chloro-4-(pyrimidin-2-yl)phenyl]amino}hexyl)-2-(hydroxymethyl)piperidine-3,4,5-triol: Purged 4-amino-3-chlorophenyl boronate (500 mg), 2-bromopyrimidine (0.8 eq), Na₂CO₃ (3 eq), toluene:EtOH:H₂O (15 mL), with Ar 30 minutes, added Pd(dppf)Cl₂·DCM (0.1 eq), reacted at 80° C. for 2 hours. Diluted with water, extracted with EtOAc, organic layer was dried over Na₂SO₄, filtered, evaporated, and purified on silica [20-30% EtOAc:hexane] to give 220 mg of 3.

Mixed 3 (220 mg), Int-1 (1.5 eq), MeOH (10 mL) at 0° C., added NaCNBH₃ (1.5 eq), AcOH (cat), reacted at room temperature for 16 hours. The volatiles were evaporated and the residue was diluted with DCM and washed with water and brine. The organic layer was separated, dried over Na₂SO₄, concentrated, and flash column purified [100-200 silica gel, eluting with 5-10% EtOAc:hexane] to give 280 mg of 4.

Mixed 4 (280 mg), 4 N HCl in dioxane (3 mL), 1,4-dioxane (9 mL) reacted 6 hours at room temperature. Volatiles were evaporated the preparative HPLC purification gave 40 mg of 1228.

Preparation of 1231 (2R,3R,4R,5S)-2-(hydroxymethyl)-1-(6-{[2-methoxy-4-(pyrimidin-2-yl)phenyl]amino}hexyl)piperidine-3,4,5-triol: Purged 4-amino-3-methoxyphenyl boronate (1.0 g), 2-bromopyrimidine (0.8 eq), Na₂CO₃ (3 eq), toluene:EtOH:H₂O (30 mL), with N₂ 30 minutes, added Pd(dppf)Cl₂·DCM (0.1 eq), reacted at 80° C. for 2 hours. The reaction was diluted with water, extracted with EtOAc, the organic layer dried over Na₂SO₄, filtered, evaporated, and purified on silica [20-30% EtOAc:hexane] to give 420 mg of 3.

3 (400 mg), Int-1 (1.5 eq), MeOH (20 mL) mixed at 0° *C, added NaCNBH₃ (1.5 eq), AcOH (cat), reacted at room temperature. Volatiles were evaporated, residue was diluted with DCM and washed with water and brine, the organic layer was separated, dried over Na₂SO₄, concentrated, and residue purified on silica [5-10% EtOAc:hexane] to give 550 mg of 4.

Mixed 4 (300 mg), 4 N dioxane:HCl (3 mL), 1,4-dioxane (9 mL) 6 hours at room temperature. Solvent was evaporated and preparative HPLC purification afforded 27 mg of 1231.

Preparation of 1229 (2R,3R,4R,5S)-1-(6-{[2-fluoro-4-(pyrimidin-2-yl)phenyl]amino}hexyl)-2-(hydroxymethyl)piperidine-3,4,5-triol: Purged 4-amino-3-fluorophenyl boronate (1.0 g), 2-bromopyrimidine (0.8 eq), Na₂CO₃ (3 eq), toluene:EtOH:H₂O (30 mL), with N₂ 30 minutes, Pd(dppf)Cl₂·DCM (0.1 eq) added, reacted at 80° C. 2 hours. Reaction was diluted with water, extracted with EtOAc which was dried over Na₂SO₄, filtered, evaporated and purified on silica [20-30% EtOAc:hexane] to give 410 mg of 3.

3 (400 mg), Int-1 (1.5 eq), MeOH (20 mL) mixed at 0° C., added NaCNBH₃ (1.5 eq), AcOH (cat.), reacted at room temperature 16 hours. The mixture was evaporated, residue was diluted with DCM which was washed with water and brine, separated, dried over Na₂SO₄, concentrated, and purified by column [5-10% EtOAc:hexane] to give 500 mg of 4.

4 (300 mg), 4 N dioxane:HCl (3 mL), 1,4-dioxane (9 mL) reacted at room temperature, 6 hours. Solvent was evaporated and preparative HPLC purification gave 30 mg 1229.

### Examples 82: Synthesis of Compounds 1227 (N-[5-(pyrimidin-2-yl)-2-({6-[(2R,3R,4R,5S)-3,4,5-trihydroxy-2-(hydroxy-methyl)piperidin-1-yl]hexyl}amino)phenyl]cyclopropanesulfonamide) and 1339 ((2R,3R,4R,5S)-2-(hydroxymethyl)-1-{6-[2-methyl-5-(pyrimidin-2-yl)-1H-1,3-benzodiazol-1-yl]hexyl}piperidine-3,4,5-triol)

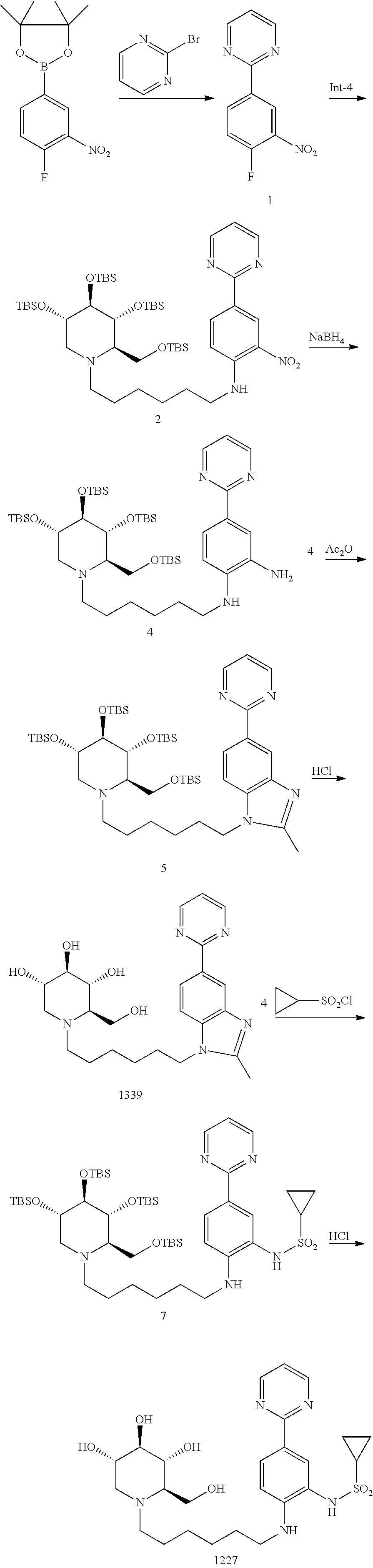

3-Nitro-4-fluorophenylboronate (4 g), 2-bromopyrimidine (1 eq), EtOH:H₂O:toluene (100 mL, 1:1:1), Na₂CO₃ (3 eq), degassed 15 minutes N₂, added Pd(dppf)Cl₂ (0.1 eq) reacted 4 hours at 80° C. The reaction was concentrated, residue was dissolved in water (50 mL) and extracted with EtOAc (2×80 mL). The organic layer was dried over Na₂SO₄, concentrated, and purified on silica [10% EtOAc:hexane] to afford 4 g of 1 as red semi-solid.

1 (500 mg), Int-4 (Example 4, 1.2 eq), 1,4-dioxane (20 mL), TEA (3 eq) reacted at 80° C. for 16 hours. The reaction was concentrated, residue was dissolved in water (20 mL), extracted with EtOAc (2×30 mL), organic was dried over Na₂SO₄, concentrated, purified on silica [10% EtOAc:hexane] to afford 1.2 g of 2 as red semi-solid.

2 (600 mg), MeOH:DCM (15 mL, 1:1), NiCl₄·6H₂O (0.1 eq), NaBH₄ (3 eq) mixed at 0° C. then at room temperature 2 hours. Reaction was quenched with ice-cold water (15 mL), extracted with EtOAc (2×20 mL), combined organic layer was dried over Na₂SO₄, filtered, concentrated, and purified on silica [30% EtOAc:hexane] to afford 300 mg of 4 as off-white solid.

Preparation of 1339 (2R,3R,4R,5S)-2-(hydroxymethyl)-1-{6-[2-methyl-5-(pyrimidin-2-yl)-1H-1,3-benzodiazol-1-yl]hexyl}piperidine-3,4,5-triol: 4 (250 mg), acetic anhydride (1.0 eq), DCM (5 mL), TEA (3 eq) mixed at 0° C. then room temperature 2 hours. The reaction was quenched with ice-cold water and extracted with DCM (2×10 mL). The organic layer was dried over Na₂SO₄, concentrated, and purified on silica [30% EtOAc:hexane] to afford 200 mg of 5 as off-white solid.

5 (200 mg), MeOH (3 mL), 4 M HCl in 1,4-dioxane (2 mL) mixed at 0° C. then room temperature for 2 hours. The solvent was evaporated then preparative HPLC purification yielded 94 mg 1339 as off-white solid.

Preparation of 1227 N-[5-(pyrimidin-2-yl)-2-({6-[(2R,3R,4R,5S)-3,4,5-trihydroxy-2-(hydroxy-methyl)piperidin-1-yl]hexyl}amino)phenyl]cyclopropanesulfonamide: Mixed 4 (300 mg), cyclopropane sulfonyl chloride (1.2 eq), DCM (5 mL), TEA (3 eq) at 0° C. then at room temperature for 16 hours. The reaction was quenched with ice-cold water and extracted with DCM (2×15 mL). The organic layer was dried over Na₂SO₄, concentrated, and purified on silica [30% EtOAc:hexane] to afford 180 mg of 7 as off-white solid.

Mixed 7 (180 mg), MeOH (3 mL), 4 M HCl in 1,4-dioxane (2 mL) at 0° C. then at room temperature for 2 hours. The solvent was evaporated and residue purified by preparative HPLC to provide 30.4 mg 1227 as off-white solid.

### Example 83: Synthesis of Compound 1236 ((2R,3R,4R,5S)-1-[6-({4-[(dimethylamino)methyl]-2-fluorophenyl}amino)hexyl]-2-(hydroxymethyl)piperidine-3,4,5-triol)

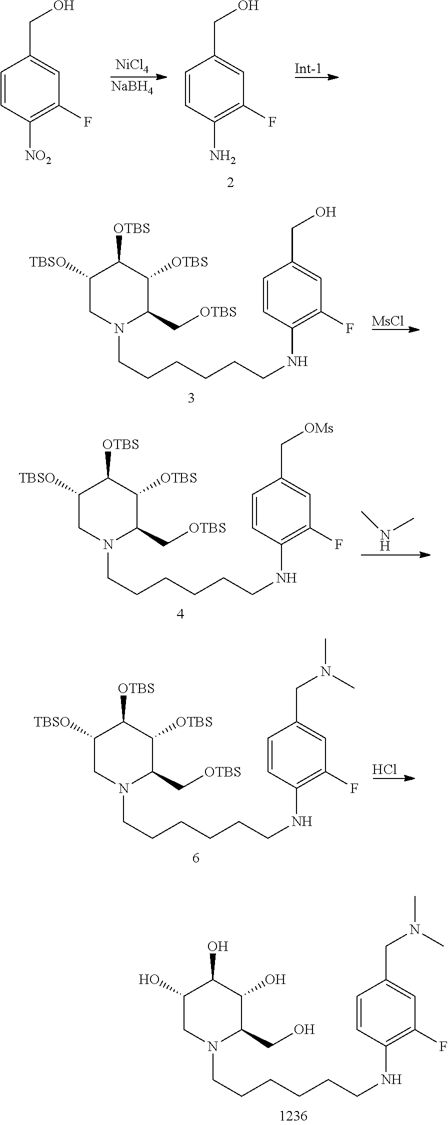

1 (1 g), MeOH:DCM (20 mL, 1:1), NiCl₄·6H₂O (0.1 eq), NaBH₄ (3 eq) mixed at 0° C., reacted at room temperature for 1 hour. The reaction was quenched with ice-cold water and diluted with DCM and washed with brine. The organic layer was separated, dried over Na₂SO₄, concentrated, and purified on silica [40% EtOAc:hexane] to afford 600 mg of 2.

Int-1 (1 g), 2 (0.8 eq), MeOH (20 mL), AcOH (cat.), NaCNBH₃ (1.5 eq) reacted at room temperature for 16 hours. The reaction was quenched with ice-cold water, diluted with DCM and washed with brine. The organic layer was separated, dried over Na₂SO₄, concentrated, and purified on silica [10% EtOAc:hexane] to afford 600 mg of 3.

Mixed 3 (600 mg), DCM (20 mL), TEA (3.0 eq), MsCl (1.2 eq) at 0° C. then at room temperature 1 hour. The reaction was quenched with ice-cold water, diluted with DCM, and washed with brine. The organic layer was dried over Na₂SO₄, concentrated to afford 500 mg of 4.

Mixed 4 (500 mg), THE (10 mL), dimethylamine (5 mL) in sealed tube at room temperature then reacted at 60° C., 4 hours. The reaction was quenched with ice-cold water and diluted with DCM and washed with brine. The organic layer was separated, dried over Na₂SO₄, concentrated, purified on silica [100% EtOAc] to afford 300 mg of 6.

Mixed 6 (300 mg), MeOH (5 mL), 4M HCl in 1,4-dioxane (3 mL) at 0° C. then at room temperature, 16 hours. Volatiles were removed and the material purified by preparative HPLC to afford 157 mg of 1236 as colorless thick syrup.

### Example 84: Synthesis of Compounds 1239 ((2R,3R,4R,5S)-1-[2-(2-{2-[(4-amino-3-chloro-2-nitrophenyl)amino]ethyl}phenyl)ethyl]-2-(hydroxymethyl)piperidine-3,4,5-triol) and 1249 ((2S,3S,4S,5R)-1-[2-(2-{2-[(4-azido-2-nitrophenyl)amino]ethyl}phenyl)ethyl]-2-(hydroxymethyl)piperidine-3,4,5-triol)

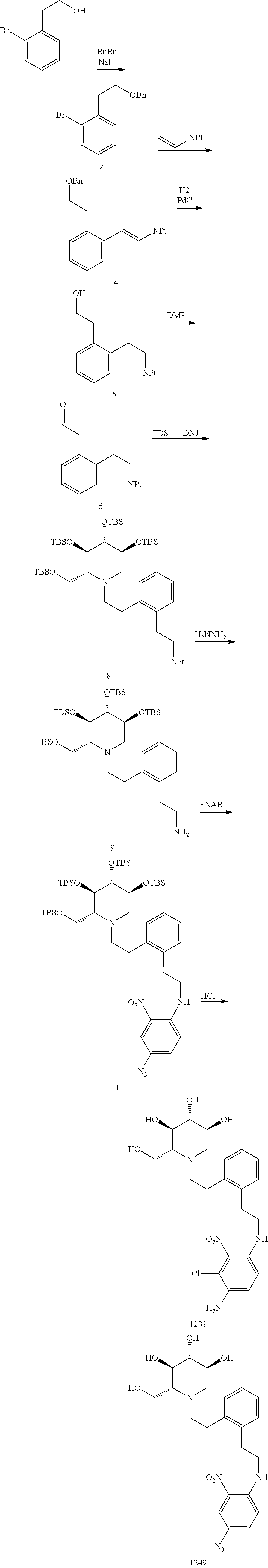

Mixed 2-(2′hydroxyethyl)bromobenzene (5 g), THF (50 mL), BnBr (1.5 eq), reacted 6 hours diluted with EtOAc, washed with water. Dried the organic over Na₂SO₄, concentrated, and purified on silica [2-5% EtOAc:hexane] to give 5.8 g of 2 as viscous liquid.

2 (1 g), HFIPA (20 mL), N-allyl phthalimide (2 eq), AgCO₃ (2 eq), acetyl glycine (0.2 eq), Pd(OAc)₂ (0.1 eq) reacted at 80° C. 12 hours. Two of these reaction mixtures was filtered through CELITE, washed with ether, evaporated, and purified on silica [10-20% EtOAc:hexane] to give 1.4 g of 4.

4 (1.4 g), EtOAc (50 mL), Pd/C (0.1 wt %) reacted under H₂ at room temperature 12 hours. The solvent was removed to afford 800 mg of 5.

5 (800 mg), DCM (15 mL), DMP (1.5 eq) reacted at room temperature, 4 hours. The reaction mixture was filtered and washed with NaHCO₃ then brine, the organic layer was dried over Na₂SO₄, concentrated, and purified on silica [15-25% EtOAc:hexane] to give 500 mg of 6.

Reacted 6 (500 mg), TBS-DNJ (1.0 g), MeOH (15 mL), NaCNBH₃ (1.5 eq), AcOH (0.1 mL) 8 hours at room temperature. Quenched with water, extracted with EtOAc, dried the organic layer over Na₂SO₄, concentrated, and purified on silica [10-15% EtOAc:hexane] to give 8 (850 mg).

8 (850 mg), EtOH (10 mL), NH₂—NH₂ (2 eq) was refluxed 6 hours. Solvent was evaporated, residue was diluted with EtOAc and washed with water and brine. The organic layer was dried over Na₂SO₄, concentrated and purified to afford 500 mg of 9.

Mixed 9 (500 mg), 1,4 dioxane (8 mL), Et₃N (3 eq), FNAB (1.1 eq) 6 hours at 80-90° C. Diluted with EtOAc, organic layer was washed with water and brine, dried over Na₂SO₄, concentrated, and purified on silica [2-5% EtOAc:hexane] to give 300 mg of 11.

11 (60 mg), 1,4 dioxane (1 mL), HCl in dioxane (0.8 mL) reacted at 0° C. After 4 hours the solvent was evaporated and the compound immediately purified by preparative HPLC to afford 12 mg of 1249.

In another batch, 11 (40 mg), 1,4 dioxane (1 mL), 4M HCl in dioxane (0.5 mL) was reacted at 0° C. for 4 hours. Solvent was evaporated and the material was purified by preparative HPLC to obtain 5 mg of 1239.

### Example 85: Synthesis of Compounds 1245 (5-[(dimethylamino)methyl]-2-({6-[(2R,3R,4R,5S)-3,4,5-trihydroxy-2-(hydroxymethyl)piperidin-1-yl]hexyl}amino)benzoic acid) and 1257 (5-[(dimethylamino)methyl]-2-({6-[(2R,3R,4R,5S)-3,4,5-trihydroxy-2-(hydroxymethyl)piperidin-1-yl]hexyl}amino)benzonitrile)

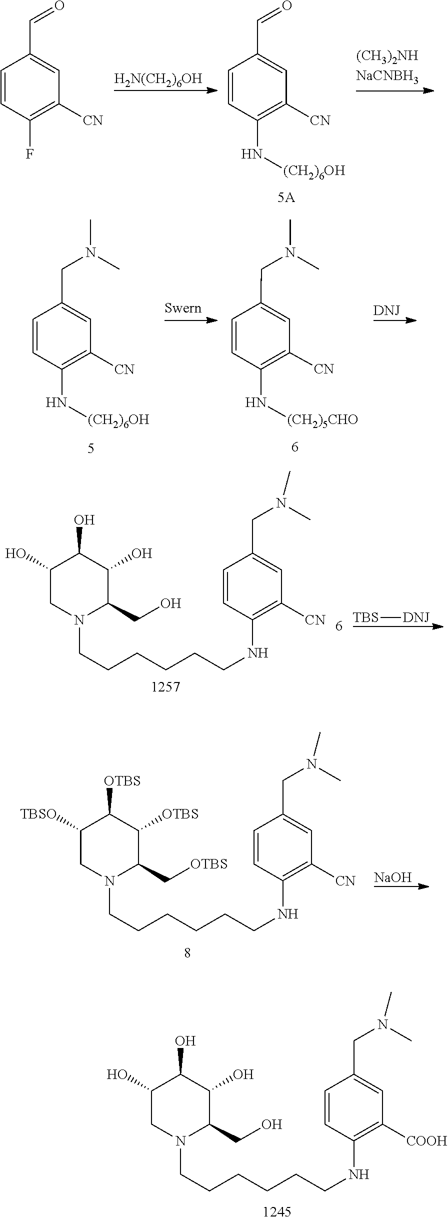

4-Fluoro-3-cyanobenzaldehyde (3 g), DMSO (30 mL), 6-aminohexan-1-ol (1.5 eq), K₂CO₃ (3 eq) mixed in sealed tube at room temperature, reacted at 80° C., 2 hours. The mixture was cooled to room temperature, quenched with ice-cold water, and extracted with EtOAc (2×50 mL). The organic layer was dried over Na₂SO₄, filtered, concentrated, and purified on silica [50% EtOAc-hexane] to afford 2.0 g of 5A as colorless thick syrup.

5A (2 g), MeOH (50 mL), dimethylamine (12 mL), NaCNBH₃ (1.5 eq) reacted at room temperature 16 hours. The mixture was quenched with ice-cold water and extracted with EtOAc (2×40 mL). The organic layer was dried over Na₂SO₄, filtered, concentrated, and purified on silica [10% MeOH-DCM, 5% NH₄OH] to afford 1 g of 5 as colorless thick syrup.

Mixed DCM (20 mL), oxalyl chloride (2.2 eq), DMSO (2.5 eq) at −78° C. for 20 minutes, added 5 (1 g) at −78° C. for 1 hour then TEA (5.5 eq) at −78° C. and increased to room temperature for 2 hours. Quenched with ice-cold water and extracted with DCM (2×30 mL). The organic layer was dried over Na₂SO₄, filtered, concentrated to afford 800 mg of 6 as colorless thick syrup.

Mixed 6 (800 mg), MeOH (20 mL), TBS-DNJ (1.0 eq), AcOH (cat.) NaCNBH₃ (1.5 eq) at room temperature for 16 hours. The reaction mixture was quenched with ice-cold water and extracted with DCM (2×30 mL). The organic layer was dried over Na₂SO₄, filtered, concentrated, and purified on silica [50% EtOAc-hexane] to afford 1.2 g of 8 as colorless thick syrup.

Preparation of 1245 5-[(dimethylamino)methyl]-2-({6-[(2R,3R,4R,5S)-3,4,5-trihydroxy-2-(hydroxymethyl)piperidin-1-yl]hexyl}amino)benzoic acid: Refluxed 8 (100 mg), EtOH (2 mL), 1N NaOH (2.5 eq) for 12 hours. Solvent was evaporated, product was diluted with DCM and neutralized with NaHSO₃. The organic layer was separated, washed with brine, dried over Na₂SO₄, evaporated, combined with another batch (50 mg input) and purified by preparative HPLC to yield 5 mg 1245.

Preparation of 1257 5-[(dimethylamino)methyl]-2-({6-[(2R,3R,4R,5S)-3,4,5-trihydroxy-2-(hydroxymethyl)piperidin-1-yl]hexyl}amino)benzonitrile: Mixed 6 (100 mg), MeOH (3 mL), DNJ (0.8 eq), AcOH (cat.) NaCNBH₃ (1.5 eq) at room temperature 16 hours. Mixture was concentrated, preparative HPLC purification gave 72 mg of 1257 as colorless thick syrup.

### Example 86: Synthesis of Compound 1259 ((2R,3R,4R,5S)-1-[6-({4-[(dimethylamino)methyl]-2-(trifluoromethyl)phenyl}amino)hexyl]-2-(hydroxymethyl)piperidine-3,4,5-triol)

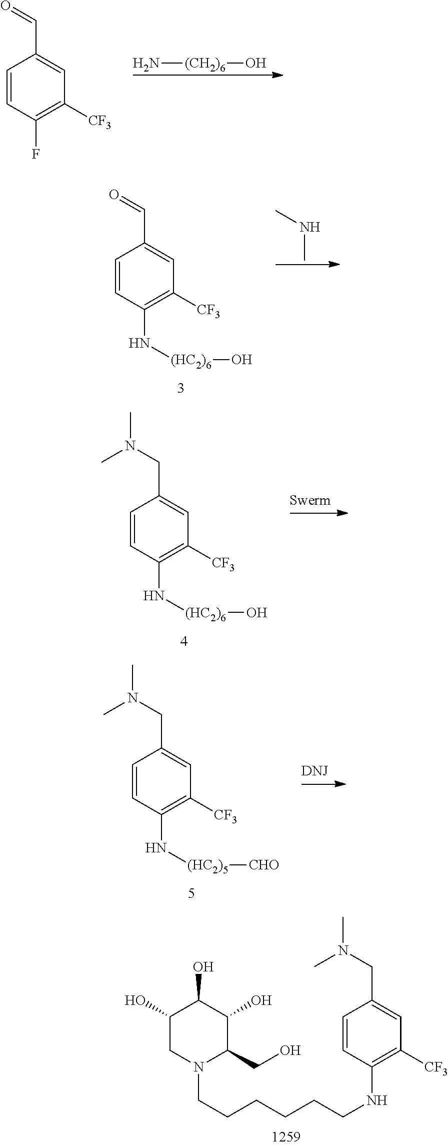

4-Fluoro-3-(trifluoromethyl)benzaldehyde (1 g), 6-aminohexan-1-ol (1.3 eq), K₂CO₃ (3 eq), DMSO (10 mL) reacted 80° C., 2 hours. Water was added, reaction was extracted with DCM, dried over Na₂SO₄, concentrated to obtain 1.05 g of 3.

3 (1 g), dimethylamine (2M in THF, 15 eq), MeOH (10 mL), AcOH (1 mL), NaCNBH₃ (1.5 eq) reacted room temperature for 16 hours. Volatiles were evaporated and material was purified by silica [5-15% MeOH in DCM] to afford 350 mg of 4.

4 (200 mg), oxalyl chloride (2.1 eq), DMSO (2.5 eq), DCM (10 mL) reacted −78° C., added TEA (5.5 eq), held at room temperature 2 hours. The reaction was quenched with water, extracted with DCM (2×50 mL), organic extracts were dried over Na₂SO₄, filtered, concentrated to give 5.

5 (160 mg), DNJ (1.2 eq), MeOH (10 mL), AcOH (0.4 mL), NaCNBH₃ (1.5 eq) reacted room temperature, 16 hours. Volatiles were evaporated. Preparative HPLC purification gave 14 mg of 1259.

### Example 87: Synthesis of Compounds 1270 ((2R,3R,4R,5S)-1-{6-[(4-azido-2-nitrophenyl)amino]-3,4-dihydroxy-hexyl}-2-(hydroxymethyl)piperidine-3,4,5-triol) and 1260 ((2R,3R,4R,5S)-1-[(3E)-6-[(4-azido-2-nitrophenyl)amino]hex-3-en-1-yl]-2-(hydroxymethyl)piperidine-3,4,5-triol)

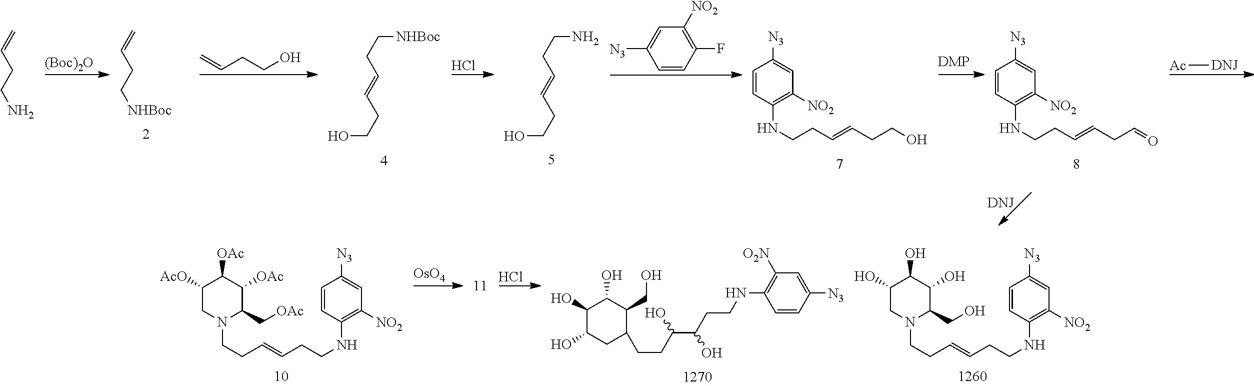

1-Aminobut-3-ene (2 g), DCM (40 mL), TEA (3 eq), (BOc)₂O (1.2 eq) reacted 16 hours at room temperature. Additional DCM was added to the reaction and the organic layer was washed with water and brine. The organic layer was dried over Na₂SO₄, filtered, concentrated, and purified on silica [10-30% EtOAc/hexane] to afford 3.0 g of 2 as colorless liquid.

2 (3.0 g), DCM (300 mL), but-3-en-1-ol (1 eq), Grubbs 2^{nd} generation catalyst (0.1 eq) reacted at room temperature 16 hours. The reaction mass was filtered on CELITE pad, washed with DCM, the organic layer was concentrated, and purified on silica [30-50% EtOAc-hexane] to afford 2 g of 4 as brown liquid.

4 (2 g), 4N 1, 4-dioxane:HCl (40 mL) reacted at room temperature 4 hours. The volatiles were concentrated to obtain 1.85 g of 5.

FNAB (1.85 g), 5 (1 eq), TEA (3 eq.), dioxane (20 mL) reacted at 80° C., 16 hours. The volatiles were removed and the material was purified on silica [30-50% EtOAc:hexane] to afford 600 mg of 7 as brown liquid.

7 (550 mg), DMP (2 eq), DCM (20 mL) reacted room temperature, 2 hours. The reaction was quenched with water then extracted with DCM (2×60 mL). Organic extracts were dried over Na₂SO₄, filtered, concentrated, purified on silica [10-20% EtOAc:hexane] to give 380 mg 8.

8 (78 mg), DNJ (1.2 eq), MeOH (5 mL), AcOH (0.2 mL), NaCNBH₃ (1.5 eq) reacted at room temperature, 16 hours. Volatiles were evaporated. Purification of this reduction with another batch (40 mg input 8) by preparative HPLC afforded 31 mg of 1260.

8 (380 mg), Ac-DNJ (1.2 eq), MeOH (10 mL), AcOH (0.4 mL), NaCNBH₃ (1.5 eq) reacted at room temperature, 16 hours. Volatiles were evaporated and the reaction mass was dissolved in DCM (50 mL) then washed with water and brine. Organic layer was dried over Na₂SO₄, filtered, concentrated, and purified on silica [20-40% EtOAc:hexane] to give 210 mg 10.

10 (185 mg), OsO₄ (0.4 M in tBuOH, 0.2 eq), NMO (3 eq), tBuOH (8 mL) reacted 4 hours at room temperature. The reaction was quenched with Na₂SO₃ solution and extracted with EtOAc. The organic layer was dried over Na₂SO₄, concentrated, and purified by preparative TLC to give 95 mg of 11 (not shown in figure). Then 11 (60 mg), aq. NH₃ (3 mL), and MeOH (1.5 mL) were reacted at room temperature 16 hours. Solvent was evaporated and two batches were purified by preparative HPLC to afford 21 mg of 1270.

### Example 88: Synthesis of Compounds 1246 (2-methyl-6-(pyrimidin-2-yl)-1-{6-[(2R,3R,4R,5S)-3,4,5-trihydroxy-2-(hydroxymethyl)piperidin-1-yl]hexyl}-1,4-dihydroquinazolin-4-one), 1250 (5-(pyrimidin-2-yl)-2-({6-[(2R,3R,4R,5S)-3,4,5-trihydroxy-2-(hydroxymethyl)piperidin-1-yl]hexyl}amino)benzoic acid), 1256 (5-(pyrimidin-2-yl)-2-({6-[(2R,3R,4R,5S)-3,4,5-trihydroxy-2-(hydroxymethyl)piperidin-1-yl]hexyl}amino)benzonitrile), and 1266 (5-(pyrimidin-2-yl)-2-({6-[(2R,3R,4R,5S)-3,4,5-trihydroxy-2-(hydroxymethyl)piperidin-1-yl]hexyl}amino)benzamide)

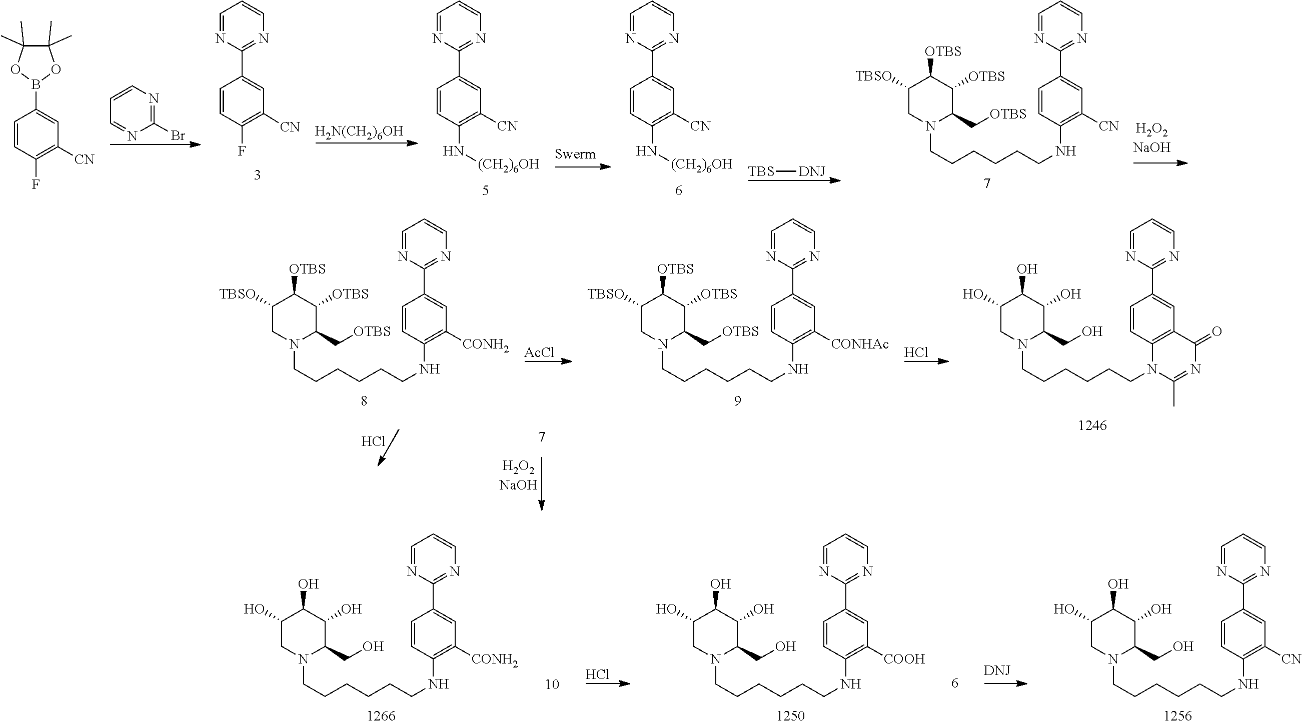

4-Fluoro-3-cyanophenylboronate (3 g) in toluene:EtOH:water (1:1:1, 60 mL) was degassed for 30 minutes, added 2-bromopyrimidine (1 eq), Na₂CO₃ (3 eq), degassed 15 minutes N₂, added Pd(dppf)Cl₂ (0.1 eq), reacted at 80° C., 2 hours. The reaction was cooled to room temperature, volatiles were removed, residue was dissolved in water and extracted with EtOAc (2×40 times). The organic layer was dried over Na₂SO₄, filtered, concentrated, and purified on silica [20% EtOAc-hexane] to afford 1.5 g of 3 as pale off-white solid.

3 (500 mg), 6-aminohexan-1-ol (1.5 eq), DMSO (5 mL), K₂CO₃ (3.0 eq) mixed at room temperature, reacted at 140° C. in sealed tube, 2 hours. The reaction was cooled to room temperature and diluted with ice-cold water (30 mL), extracted with EtOAc (60 mL) and washed with water (30 mL). The organic layer was dried over Na₂SO₄, filtered, concentrated, and purified on silica [10% EtOAc:hexane] to afford 320 mg of 5 as off-white solid.

5 (1.2 g), DCM (25 mL), (COCl)₂ (2.1 eq), DMSO (2.6 eq) were mixed at −78° C. for 2 hours TEA (5.4 eq) added and the reaction was quenched with ice cold water (20 mL) and extracted with DCM (2×30 mL). The combined organic layer was dried over Na₂SO₄, filtered, and concentrated to afford 1 g of 6 as colorless thick syrup.

6 (1 g), MeOH (20 mL), TBS-DNJ (0.8 eq), AcOH (cat.), NaCNBH₃ (1.5 eq) reacted at room temperature 16 hours. Volatiles were evaporated and residue was purified on silica [10% EtOAc-hexane] to afford 2.0 g of 7 as colorless thick syrup.

Mixed 7 (500 mg), DMSO:MeOH (10 mL), 1N NaOH (1 eq), 30% H₂O₂ (1.5 eq) at 0° C., then 6 hours at room temperature. Quenched with ice cold water (20 mL) and extracted with EtOAc (2×20 mL). The combined organic layer was dried over Na₂SO₄, filtered, concentrated, and purified on silica [40% EtOAc-hexane] to afford 400 mg of 8 as colorless thick syrup.

Preparation of 1266 5-(pyrimidin-2-yl)-2-({6-[(2R,3R,4R,5S)-3,4,5-trihydroxy-2-(hydroxymethyl)piperidin-1-yl]hexyl}amino)benzamide: 8 (200 mg), MeOH (50 mL), 4 M HCl in 1,4-dioxane (1 mL) mixed at 0° C., increased to room temperature for 16 hours. The volatiles were concentrated and residue after preparative HPLC purification yielded 46 mg of 1266.

Preparation of 1246 2-methyl-6-(pyrimidin-2-yl)-1-{6-[(2R,3R,4R,5S)-3,4,5-trihydroxy-2-(hydroxymethyl)piperidin-1-yl]hexyl}-1,4-dihydroquinazolin-4-one: Mixed 8 (100 mg), DCM:pyridine (5 mL, 1:1), acetyl chloride (1.5 eq) at 0° C. then 2 hours at room temperature. Quenched with ice cold water (10 mL), extracted with EtOAc (2×10 mL), dried the combined organic layer over Na₂SO₄, filtered, concentrated, and purified on silica [50% EtOAc-hexane] to afford 20 mg of 9 as colorless thick syrup.

Mixed 9 (20 mg), MeOH (2 mL), 4M HCl in 1,4-dioxane (0.2 mL) at 0° C. then at room temperature for 16 hours. Volatiles were concentrated, preparative HPLC purification gave 4 mg 1246.

Preparation of 1250 5-(pyrimidin-2-yl)-2-({6-[(2R,3R,4R,5S)-3,4,5-trihydroxy-2-(hydroxymethyl)piperidin-1-yl]hexyl}amino)benzoic acid: 7 (500 mg), EtOH:H₂O (10 mL), NaOH (2.5 eq) mixed at room temperature then at 100° C. for 16 hours. The reaction was quenched with saturated NaHSO₃ solution (25 mL) and extracted with DCM (2×20 mL). The organic layer was dried over Na₂SO₄, filtered, concentrated, and purified on silica [30% EtOAc/hexane] to afford 250 mg of the benzoic acid (10, not shown in figure) as colorless thick syrup.

10 (300 mg), 1,4-dioxane (10 mL), 4M HCl in 1,4-dioxane (2.5 mL) mixed at 0° C. then room temperature for 12 hours. Volatiles were removed and after preparative HPLC purification 28 mg of 1250 was obtained.

Preparation of 1256 5-(pyrimidin-2-yl)-2-({6-[(2R,3R,4R,5S)-3,4,5-trihydroxy-2-(hydroxymethyl)piperidin-1-yl]hexyl}amino)benzonitrile: 6 (200 mg), MeOH (5 mL), DNJ (0.8 eq), AcOH (cat.), NaCNBH₃ (1.5 eq) reacted at room temperature, 16 hours. Volatiles were concentrated and the material was purified by preparative HPLC to afford 36.1 mg of 1256 as white solid.

### Example 89: Synthesis of Compounds 1265 (5-(pyrimidin-2-yl)-2-({6-[(2R,3R,4R,5S)-3,4,5-trihydroxy-2-(hydroxymethyl)piperidin-1-yl]hexyl}amino)benzene-1-sulfonamide) and 1247 (N-(dimethyl-2-sulfanylidene)-5-(pyrimidin-2-yl)-2-({6-[(2R,3R,4R,5S)-3,4,5-trihydroxy-2-(hydroxymethyl)piperidin-1-yl]hexyl}amino)benzene-1-sulfonamide)

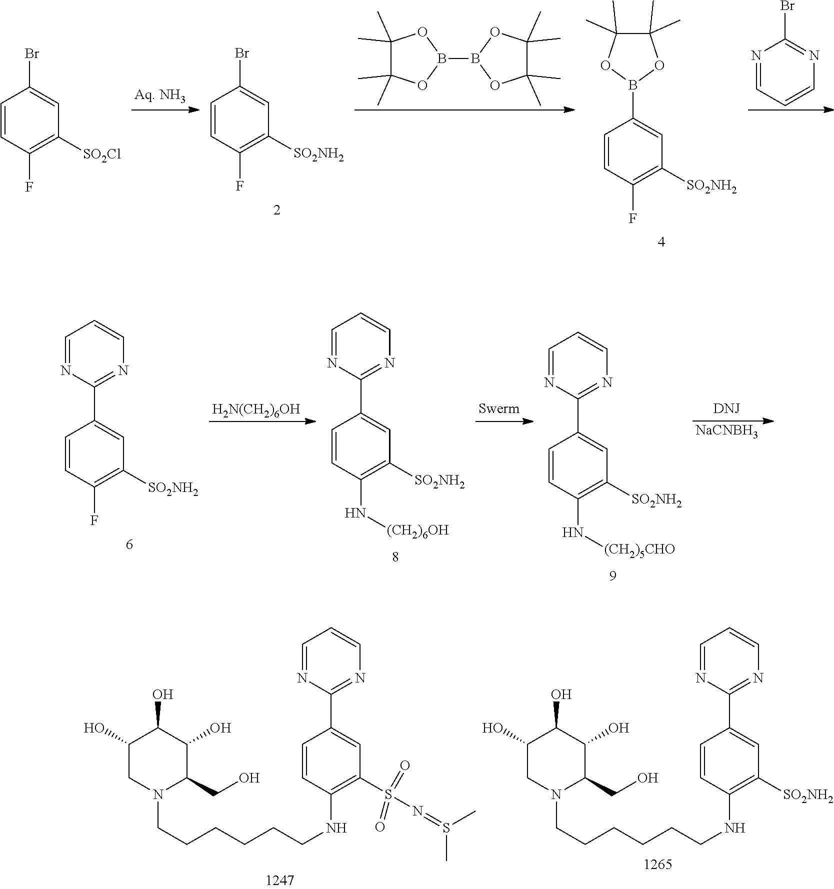

1-Fluoro-4-bromobenzenesulfonyl chloride (3 g), 1,4-dioxane (50 mL), aqueous NH₃ (25 mL) reacted 16 hours. The reaction was cooled to room temperature, volatiles were removed, residue was dissolved in water and extracted with EtOAc (2×100 mL). The organic layer was dried over Na₂SO₄, filtered, and concentrated to afford 2.4 g of 2.

Degassed 2 (2.4 g), bis(pinacolato) diboron (1.5 eq), KOAc (3 eq), 1,4-dioxane (5 mL) 30 minutes, added Pd (dppf)Cl₂·DCM (0.029 mmol), reacted at 80° C. 6 hours. Cooled, diluted with hexane (100 mL), stirred for 10 minutes, filtered through CELITE bed, washed with hexane. Evaporated filtrate and dissolved residue in water, extracted with EtOAc (2×50 mL). The organic layer was dried over Na₂SO₄, filtered, and concentrated to afford 2.4 g of 4.

4 (2.4 g) in toluene:EtOH:water (1:1:1, 60 mL) was degassed for 30 minutes, added 2-bromo-pyrimidine (1 eq), Na₂CO₃ (3 eq), degassed 15 minutes N₂, added Pd(dppf)Cl₂ (0.1 eq), reacted 80° C. for 7 hours. Reaction was cooled to room temperature, volatiles were removed, residue was dissolved in water and extracted with EtOAc (2×50 mL). The organic layer was dried over Na₂SO₄, filtered, concentrated, and purified on silica [40% EtOAc-hexane] to afford 1.1 g of 6 as pale brown solid.

6 (1 g), DMSO (20 mL), 6-aminohexan-1-ol (2 eq), K₂CO₃ (3 eq) reacted in sealed tube at 150° C. for 3 hours. Reaction was cooled to room temperature; quenched with ice-cold water (100 mL) and stirred for 10 minutes. The obtained solid was filtered and washed with water. The product was dried under vacuum to afford 1 g of 8 as pale brown solid.

8 (500 mg), THE (50 mL), (COCl)₂ (3 eq), DMSO (4 eq) reacted at −78° C. 2 hours, TEA (8.5 eq) added and increased to room temperature. The reaction was quenched with ice cold water (50 mL) and extracted with DCM (2×30 mL). The organic layer was dried over Na₂SO₄ filtered, concentrated to afford 500 mg of 9 as pale brown syrup.

9 (500 mg), DNJ (1 eq), MeOH (30 mL), AcOH (0.4 mL), NaCNBH₃ (2 eq) reacted at room temperature, 12 hours. Volatiles were evaporated and preparative HPLC purification afforded 10 mg of 1265 and 15 mg of 1247.

### Example 90: Synthesis of Compound 1258 ((2R,3R,4R,5S)-2-(hydroxymethyl)-1-(6-{[4-(pyrimidin-2-yl)-2-(trifluoromethyl)phenyl]amino}hexyl)piperidine-3,4,5-triol)

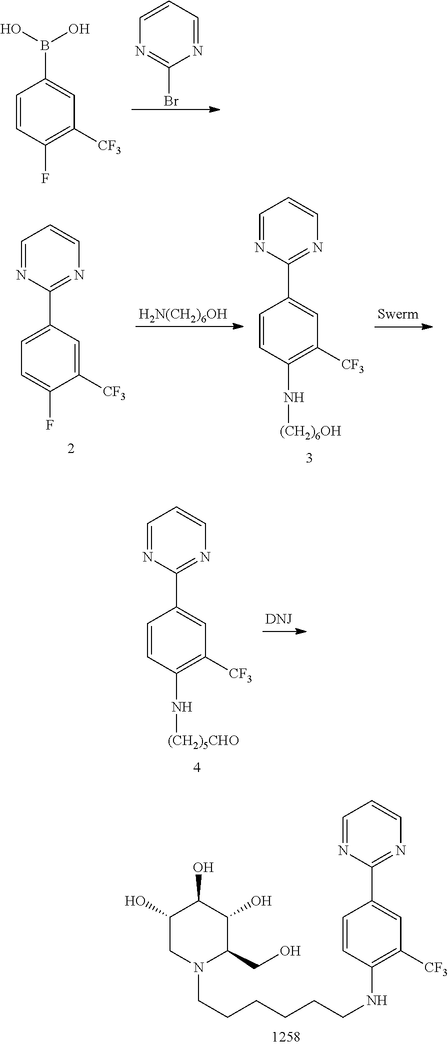

2-Bromopyrimidine (800 mg), toluene:water:ethanol (1:1:1; 30 mL), Na₂CO₃ (3.0 eq), 4-fluoro-3-trifluoromethylphenylboronic acid (1 eq) reacted 8 hours at 80° C. The reaction was cooled to room temperature and concentrated, residue was dissolved in EtOAc (100 mL) and washed with water (30 mL). The organic layer was dried over Na₂SO₄, filtered, concentrated, and purified on silica [10% EtOAc-hexane] to afford 1 g of 2 as off-white solid.

2 (1 g), DMSO (15 mL), 6-aminohexan-1-ol (2.0 eq), K₂CO₃ (3 eq) reacted at 140° C., 4 hours. The reaction was cooled to room temperature, quenched with ice-cold water, the aqueous layer was extracted with EtOAc (2×15 mL). The organic layer was dried over Na₂SO₄, filtered, concentrated, and purified on silica [20% EtOAc-hexane] to afford 800 mg of 3.

Mixed 3 (200 mg), DCM (10 mL), (COCl)₂ (3 eq), DMSO (4 eq) at −78° C. for 2 hours, added TEA (8 eq), quenched with ice cold water (20 mL), extracted with DCM (2×10 mL). The organic layer was dried over Na₂SO₄ filtered, concentrated to afford 190 mg of 4.

Mixed 4 (190 mg), MeOH, DNJ (1 eq), AcOH at 0° C., added NaCNBH₃ (2 eq) reacted at room temperature 12 hours. Concentrated solvents then preparative HPLC purification gave 45 mg of 1258.

### Example 91: Synthesis of Compound 1240 ((2R,3R,4R,5S)-1-[2-(3-{2-[(4-azido-2-nitrophenyl)amino]ethyl}phenyl)ethyl]-2-(hydroxymethyl)piperidine-3,4,5-triol)

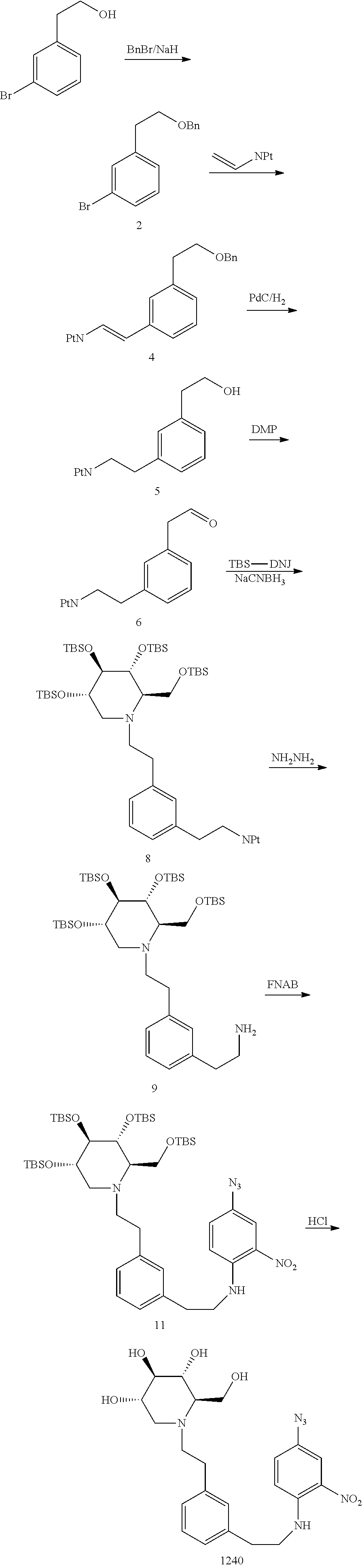

To 12 g of 2-(3-bromophenyl)ethan-1-ol in 300 mL THE at 0° C. added NaH (1.5 eq), BnBr (0.9 eq), TBAI (cat.), raised to room temperature, mixed 16 hours. Quenched the reaction with ice-cold water and extracted with EtOAc (2×100 mL). The organic layer was dried over Na₂SO₄, filtered, concentrated, and purified on silica [5% EtOAc in hexane] to afford 12 g of 2.

2 (5 g), N-allylphthalimide (1.2 eq), 1,1,1,3,3,3-hexafluoro-2-propanol (80 mL), Ag₂CO₃ (2 eq), acetyl glycine (0.2 eq), Pd(OAc)₂ (0.2 eq) reacted at 80° C. 16 hours. The reaction was cooled to room temperature, filtered through CELITE bed and washed with DCM (300 mL). The filtrate was concentrated and purified on silica [5% EtOAc in hexane] to afford 2.8 g of 4.

4 (2.8 g), EtOAc (100 mL), 10% Pd/C (50% wet, 2.4 g) reduced under H₂ at room temperature for 24 hours. The reaction was filtered through CELITE bed and washed with EtOAc (400 mL). The filtrate was distilled and purified on silica [40% EtOAc in hexane] to afford 1.7 g of 5.

Mixed 5 (1.7 g), DCM (70 mL), DMP (1.5 eq) at room temperature 2 hours. Quenched with saturated aqueous NaHCO₃ (100 mL) and extracted with DCM (2×100 mL). The organic layer was dried over Na₂SO₄, filtered, and purified on silica [20% EtOAc in hexane] to afford 1.4 g of 6.

To 6 (1.4 g), MeOH (50 mL), TBS-DNJ (0.95 eq), AcOH (cat) at 0° C., added NaCNBH₃ (1.5 eq) reacted at room temperature 16 hours. The reaction was concentrated, residue was dissolved in water (100 mL) and extracted with EtOAc (2×100 mL). The organic layer dried over Na₂SO₄, filtered, concentrated, and purified on silica [20% EtOAc in hexane] to afford 2 g of 8.

8 (2 g), EtOH (50 mL), NH₂·NH₂·H₂O (20 mL), mixed room temperature for 16 hours. The reaction was concentrated. Residue was dissolved in water (100 mL) and extracted with EtOAc (2×100 mL). The organic layer dried over Na₂SO₄, filtered, concentrated to obtain 1.2 g of 9.

9 (1.2 g), 1,4-dioxane (20 mL), TEA (3 eq), FNAB (1.1 eq), reacted 16 hours at 80° C. The reaction was concentrated. Residue was dissolved in water (50 mL) and extracted with EtOAc (2×100 mL). The organic layer was dried over Na₂SO₄, filtered, concentrated, and purified on silica [10% EtOAc in hexane] to afford 600 mg of 11.

11 (300 mg), dioxane (5 mL), 4.0 M HCl in 1,4-dioxane (5 mL) reacted room temperature for 8 hours. Solvent removal and preparative HPLC purification yielded 41 mg of 1240.

### Example 92: Synthesis of Compound 1275 ((2R,3R,4R,5S)-1-{2-[({2-[(4-azido-2-nitrophenyl)amino]ethyl}sulfamoyl)amino]ethyl}-2-(hydroxymethyl)piperidine-3,4,5-triol)

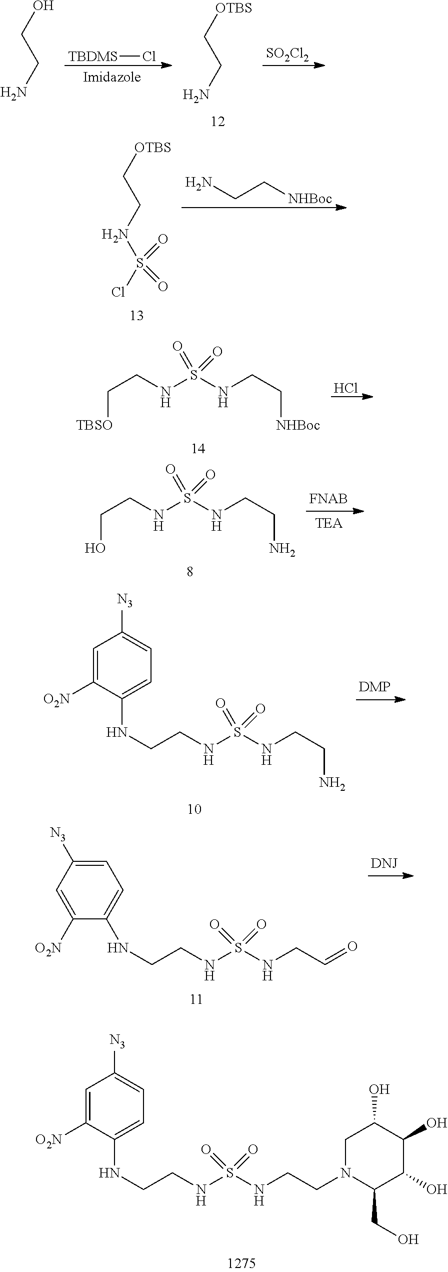

Ethanolamine (5 g), DCM (80 mL), imidazole (2 eq), TBDMS-Cl (0.9 eq) mixed at room temperature 2 hours. The reaction was quenched with ice-cold water and extracted with DCM (2×200 mL). The organic layer was dried over Na₂SO₄, filtered, concentrated to afford 6 g of 12.

To 12 (1 g) in DCM (12 mL) at −15° C. added TEA (4 eq) and SO₂Cl₂ (2 eq). After addition of SO₂Cl₂ the mix was added to a pre-mixed solution of mono-N-Boc-1,2-diaminoethane (1 eq) in 25 mL DCM at 0° C. The mix was warmed to room temperature and stirred 16 hours. The reaction was quenched with ice-cold water and extracted with DCM (2×50 mL). The organic layer was dried over Na₂SO₄, filtered, concentrated, and purified on silica [20% EtOAc:hexane] to afford 400 mg of 14 as colorless thick syrup.

14 (400 mg), MeOH (7 mL), 4.0 M HCl 1,4-dioxane (10 mL) reacted 16 hours at room temperature. The solvent was concentrated to afford 250 mg of 8.

8 (250 mg), 1,4-dioxane (10 mL), TEA (10 eq), FNAB (1 eq) reacted at 80° C. 16 hours. The reaction was concentrated, the residue dissolved in water and extracted EtOAc (2×10 mL). The organic layer was dried over Na₂SO₄, filtered, concentrated, and purified on silica [70% EtOAc:hexane] to provide 150 mg of 10 as red solid.

Mixed 10 (90 mg), DCM (20 mL) at 0° C., added DMP (2 eq) at room temperature 8 hours. Quenched with saturated aqueous NaHCO₃ and extracted with DCM (2×20 mL). The organic layer was dried over Na₂SO₄, filtered, and concentrated to afford 50 mg of 11.

Mixed 11 (90 mg), MeOH (10 mL), DNJ (1 eq), AcOH (cat.) at 0° C., added NaCNBH₃ (1.5 eq) mixed at room temperature for 4 hours. Preparative HPLC purification gave 26 mg of 1275.

### Example 93: Synthesis of compounds 1271 (2R,3R,4R,5S)-1-{2-[(1-{2-[(4-azido-2-nitrophenyl)amino]ethyl}azetidin-3-yl)oxy]ethyl}-2-(hydroxymethyl)piperidine-3,4,5-triol and 1252 (2R,3R,4R,5S)-1-(2-{[1-(4-azido-2-nitrophenyl)azetidin-3-yl]oxy}ethyl)-2-(hydroxymethyl)piperidine-3,4,5-triol

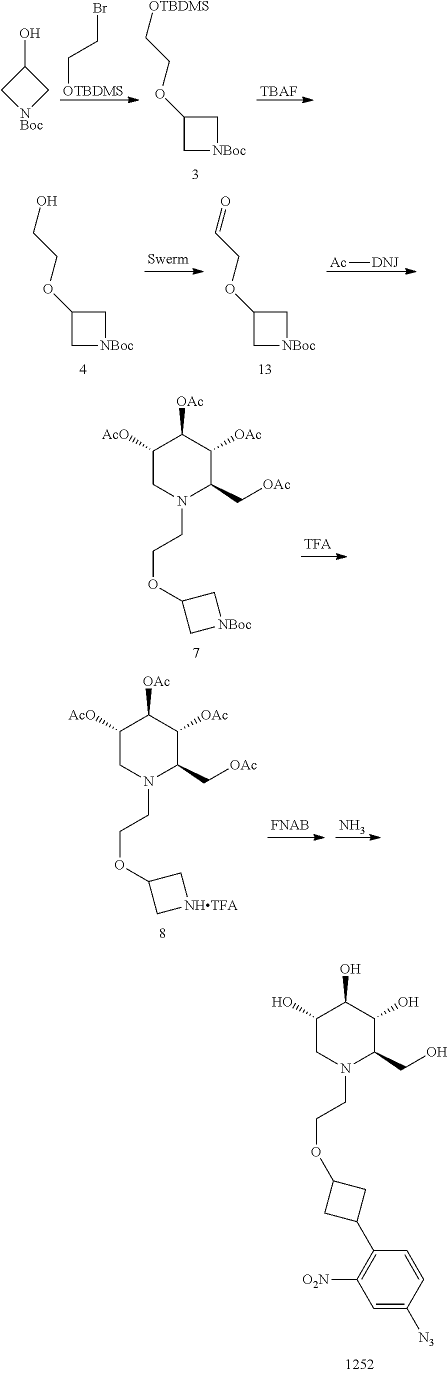

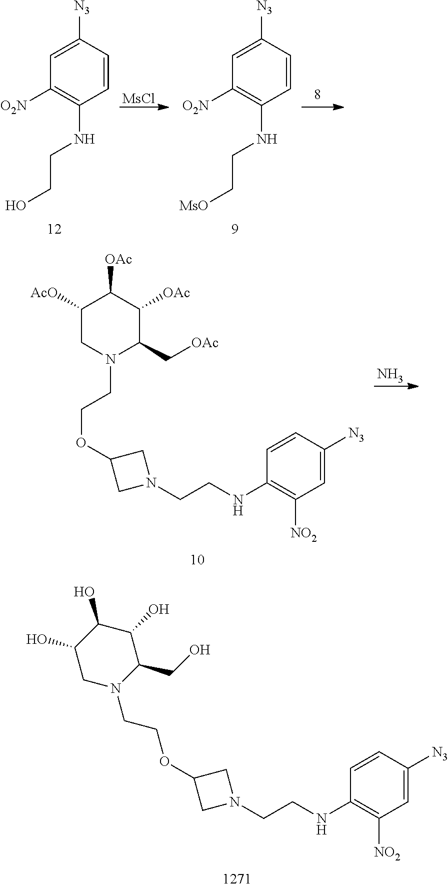

Mixed N-Boc-3-hydroxyazetidine (4 g), DMF (50 mL) at 0° C., added NaH (2 eq) mixed at room temperature 20 minutes, added TBDMS-protected bromoethanol (3 eq) increased to 80° C. for 12 hours. Cooled to room temperature, quenched with ice-cold water (200 mL), extracted with EtOAc (2×150 mL). Organic layer was washed with water (2×100 mL), dried over Na₂SO₄, filtered, concentrated, and purified on silica [50% EtOAc in DCM] to afford 2.4 g of 3.

To 3 (2.4 g) in THE at 0° C. added 1.0 M TBAF in THE (1.5 eq), reacted at 0° C., 2 hours. Reaction was quenched with ice-cold water (50 mL) and extracted with EtOAc (2×50 mL). The organic layer was dried over Na₂SO₄, filtered, concentrated, and purified on silica [20% EtOAc in hexane] to afford 1.2 g of 4 as pale yellow liquid.

4 (1.2 g), DCM (70 mL), oxalyl chloride (2 eq), DMSO (3.6 eq) reacted −78° C. for 2 hours, added TEA (8.5 eq), quenched with ice-cold water and extracted with DCM (2×10 mL). The organic layer was dried over Na₂SO₄, filtered, concentrated to afford 1.3 g of 13.

13 (1.3 g), MeOH (20 mL), Ac-DNJ (1 eq), AcOH (1 mL) mixed at 0° C., NaCNBH₃ (1.5 eq) added then maintained 12 hours at room temperature. Solvent was removed and the residue was diluted with water (50 mL) and extracted with EtOAc (2×20 mL). The organic layer was dried over anhydrous Na₂SO₄, filtered, concentrated, and purified on silica [20% EtOAc in hexane] to afford 650 mg of 7 as pale brown semi solid.

7 (650 mg) in DCM (20 mL) with TFA (8 eq) reacted 24 hours at room temperature. The reaction was concentrated to afford 400 mg 8.

12 (600 mg, Example 98), DCM, TEA (3 eq), MsCl (2 eq) reacted at room temperature, 2 hours. The reaction was quenched with ice-cold water then extracted with DCM (2×15 mL). The organic layer was dried over anhydrous Na₂SO₄, filtered, concentrated to afford 600 mg of 9.

Preparation of 1271 (2R,3R,4R,5S)-1-{2-[(1-{2-[(4-azido-2-nitrophenyl)amino]ethyl}azetidin-3-yl)oxy]ethyl}-2-(hydroxymethyl)piperidine-3,4,5-triol: To 8 (400 mg) in ACN (15 mL) added 9 (1 eq) and K₂CO₃ (6 eq), reacted at 80° C., 12 hours. Cooled to room temperature, evaporated solvent, dissolved residue in ice cold water (30 mL) and extracted with EtOAc (2×20 mL). The organic layer was dried over Na₂SO₄, filtered, concentrated, and purified on silica [80% EtOAc-hexane] to afford 10.

10 (178 mg), MeOH (10 mL), aq. NH₃ (10 mL) reacted room temperature, 12 hours. Solvent was removed and the material was purified by preparative HPLC to afford 40 mg of 1271.

Preparation of 1252 (2R,3R,4R,5S)-1-(2-{[1-(4-azido-2-nitrophenyl)azetidin-3-yl]oxy}ethyl)-2-(hydroxymethyl)piperidine-3,4,5-triol: Reacted 8 (150 mg), 1,4-dioxane (5 mL), FNAB (1 eq), TEA (8 eq) at 80° C. 12 hours. Removed solvent, diluted residue with water (10 mL) and extracted with EtOAc (2×10 mL). Dried the organic layer over Na₂SO₄, filtered, concentrated, and purified on silica [40% EtOAc hexane] to afford 135 mg of pale brown syrup. Cooled to 0° C., added methanol and aq. NH₃ (10 mL), reacted at room temperature, 12 hours. Reaction was concentrated and preparative HPLC purification gave 30 mg of 1252.

### Example 94: Synthesis of Compound 1251 (4-fluoro-2-({6-[(2R,3R,4R,5S)-3,4,5-trihydroxy-2-(hydroxymethyl)piperidin-1-yl]hexyl}amino)benzonitrile)

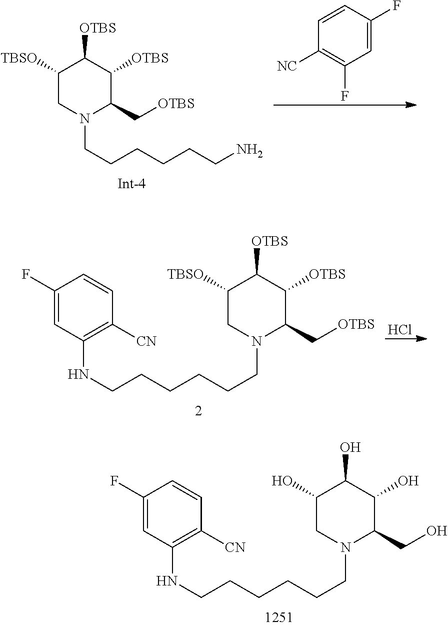 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCCCCCN ; N#Cc1ccc(F)cc1F ; CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCCCCCNc1cc(F)ccc1C#N ; Cl ; N#Cc1ccc(F)cc1NCCCCCCN1C[C@H](O)[C@@H](O)[C@H](O)[C@H]1CO]

Int-4 (300 mg, Example 4), DMF (3 mL), 2,4-difluorobenzonitrile (0.8 eq), K₂CO₃ (3.0 eq) mixed at room temperature then at 100° C. for 2 hours. The reaction was cooled to room temperature and diluted with ice-cold water, extracted with EtOAc (2×20 mL). Organic layers were washed with water (10 mL), dried over Na₂SO₄, filtered, concentrated, purified on silica [10% EtOAc-hexane] to afford 150 mg of 2 as colorless thick syrup.

2 (150 mg), 1,4-dioxane (5 mL), 4M HCl in 1,4-dioxane (2 mL), mixed at 0° C. then room temperature, 16 hours. Reaction mass was concentrated then preparative HPLC purification afforded 43.4 mg of 1251 as colorless thick syrup.

### Example 95: Synthesis of Compounds 1273 ((2R,3R,4R,5S)-1-[2-(4-{2-[(4-azido-2-nitrophenyl)amino]ethyl}piperazin-1-yl)ethyl]-2-(hydroxymethyl)piperidine-3,4,5-triol) and 1254 ((2R,3R,4R,5S)-1-{2-[4-(4-azido-2-nitrophenyl)piperazin-1-yl]ethyl}-2-(hydroxymethyl)piperidine-3,4,5-triol)

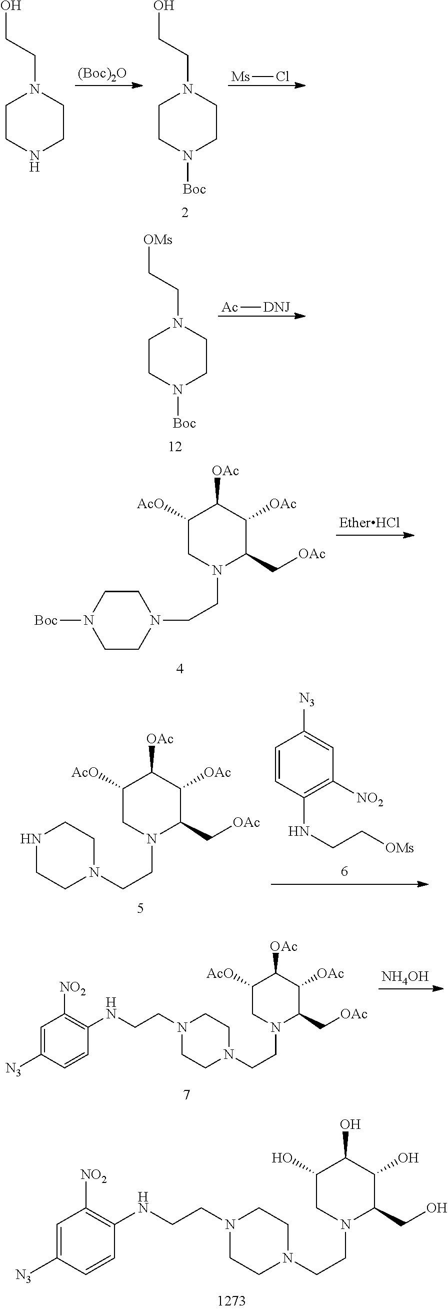

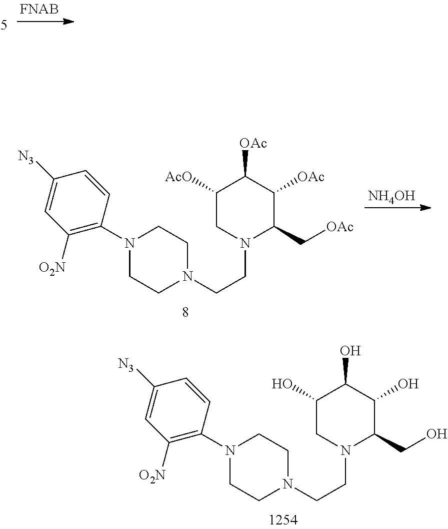 [SMILES: CC(=O)OC[C@@H]1[C@@H](OC(C)=O)[C@H](OC(C)=O)[C@@H](OC(C)=O)CN1CCN1CCN(c2ccc(N=[N+]=[N-])cc2[N+](=O)[O-])CC1 ; "NH4OH" ; [N-]=[N+]=Nc1ccc(N2CCN(CCN3C[C@H](O)[C@@H](O)[C@H](O)[C@H]3CO)CC2)c([N+](=O)[O-])c1]

Mixed 1-(2-hydroxyethyl)piperidine (6 g), DCM (50 mL), TEA (3.0 eq), (Boc)₂O (1.2 eq) at 0° C. then room temperature 16 hours. Diluted with water (100 mL), organic layer was dried over anhydrous Na₂SO₄, filtered and concentrated to afford 7 g of 2 as colorless thick syrup.

2 (3 g), DCM, TEA (2 eq), mesyl chloride (1.5 eq) reacted at room temperature, 2 hours. The reaction was quenched with ice cold water (10 mL), extracted with DCM (2×10 mL), organic layer was dried over Na₂SO₄, filtered, and concentrated to afford 3 g of 12.

12 (3 g), THF, Ac-DNJ (1 eq.), NaHCO₃ (4 eq) reacted in sealed tube at 100° C., 12 hours. The reaction was concentrated and residue was dissolved in EtOAc (200 mL) and washed with water (300 mL). Organic layer was dried over Na₂SO₄, filtered, concentrated, and purified on silica [50% EtOAc in DCM] to afford 2 g of 4.

4 (500 mg), DCM (5 mL), HCl in diethyl ether (5 mL), reacted at 0° C., 2 hours. The reaction was diluted with Et₂₀:pentane (1:1, 20 mL) and stirred for 10 minutes. The obtained solid was filtered, washed with pentane, dried under reduced pressure to obtain 300 mg of 5.

5 (220 mg), ACN (20 mL), 6 (1 eq. prepared as in Example 93), K₂CO₃ (5 eq) reacted 24 hours at 80° C. The reaction was concentrated, residue was dissolved in EtOAc (200 mL) and washed with water (300 mL). Organic layer was dried over Na₂SO₄, filtered, concentrated, and purified on silica [50% EtOAc in DCM] to afford 110 mg of 7.

Preparation of 1273 (2R,3R,4R,5S)-1-[2-(4-{2-[(4-azido-2-nitrophenyl)amino]ethyl}piperazin-1-yl)ethyl]-2-(hydroxymethyl)piperidine-3,4,5-triol: 7 (120 mg), MeOH (5 mL), aqueous NH₃ (5 mL) reacted in sealed tube at room temperature, 12 hours. The reaction was concentrated then purified by preparative HPLC to give 8 mg 1273.

Preparation of 1254 (2R,3R,4R,5S)-1-{2-[4-(4-azido-2-nitrophenyl)piperazin-1-yl]ethyl}-2-(hydroxymethyl)piperidine-3,4,5-triol: Reacted 5 (250 mg), 1,4-dioxane (5 mL), FNAB (1 eq), TEA (8 eq) at 80° C., 12 hours. Concentrated the reaction mixture, dissolved residue in EtOAc (10 mL), washed with water (20 mL). Dried the organic layer over Na₂SO₄, filtered, concentrated, and purified by column [30% EtOAc in DCM] to afford 150 mg of 8.

8 (170 mg), MeOH (10 mL), 23% aqueous NH₃ (5 mL) reacted in sealed tube at room temperature, 12 hours. Reaction mixture was concentrated then preparative HPLC gave 50 mg of 1254.

### Example 96: Synthesis of Compounds 1272 ((2R,3R,4R,5S)-1-[2-(1-{2-[(4-azido-2-nitrophenyl)amino]ethyl}-1H-1,2,3-triazol-4-yl)ethyl]-2-(hydroxymethyl)piperidine-3,4,5-triol) and 1253 ((2R,3R,4R,5S)-1-[2-(1-{2-[(4-amino-3-chloro-2-nitrophenyl)amino]ethyl}-1H-1,2,3-triazol-4-yl)ethyl]-2-(hydroxymethyl)piperidine-3,4,5-triol)

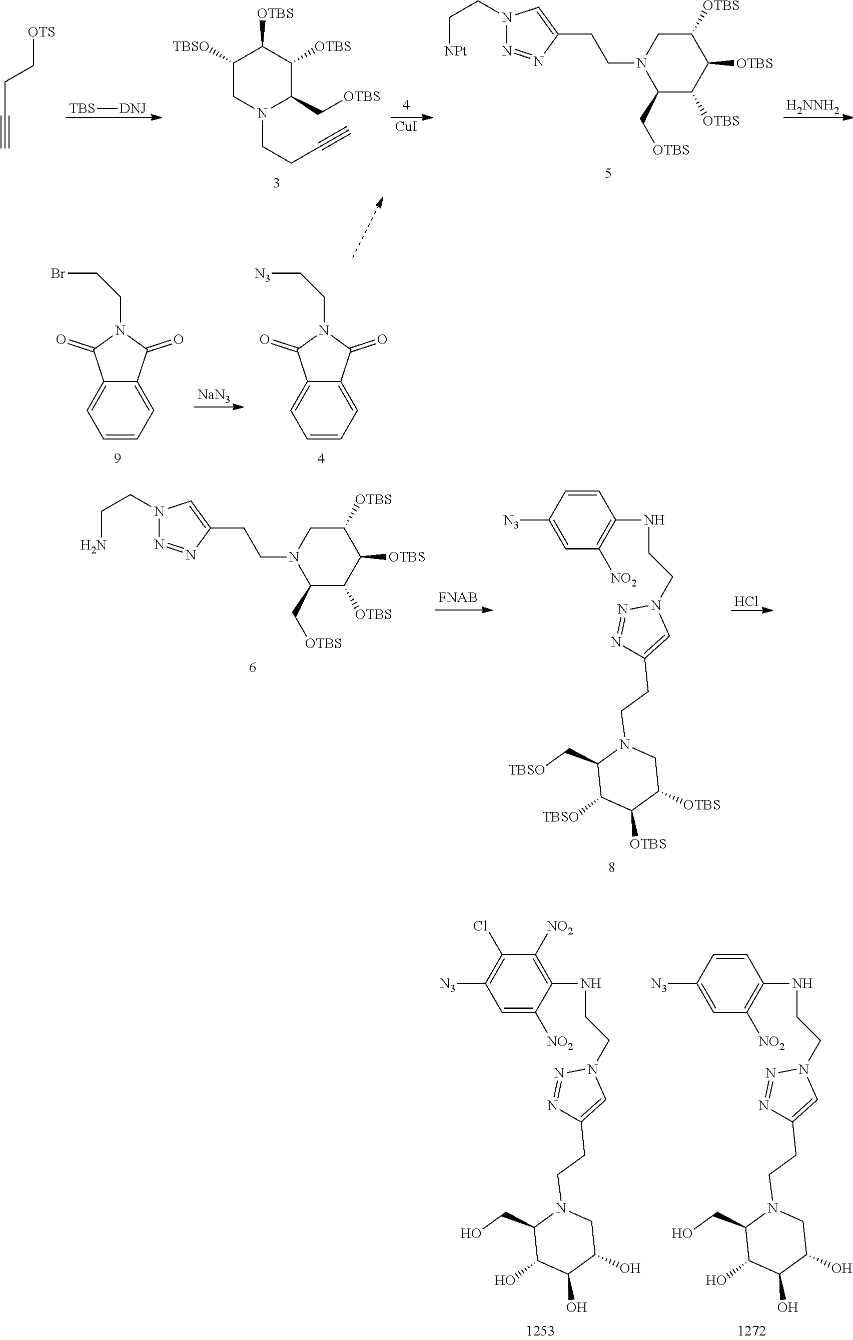

Mixed TBS-DNJ (3.0 g), ACN (60 mL), but-3-yn-1-yl 4-methylbenzenesulfonate (1.2 eq), K₂CO₃ (3.0 eq) at room temperature then at 80° C. 24 hours. Reaction was quenched with ice-cold water (50 mL), extracted with DCM (2×70 mL), the organic layer was dried over Na₂SO₄, filtered, concentrated, and purified on silica [5% EtOAc-hexane] to afford 2.5 g of 3.

2-(2-bromoethyl)isoindoline-1,3-dione (5 g), DMSO (20 mL), NaN₃ (3 eq) reacted 16 hours at room temperature. The reaction was quenched with ice-cold water, extracted with EtOAc (2×120 mL), organic layer was washed with water, dried over Na₂SO₄, filtered, concentrated, and purified on silica [30% EtOAc in hexane] to afford 4 g of 4 as white solid.

3 (2.5 g), toluene (50 mL), 4 (1 eq), CuI (0.1 eq) mixed at room temperature then at 80° C. 16 hours. The reaction was quenched with ice cold water (40 mL), extracted with EtOAc (2×75 mL), organic layer was dried over Na₂SO₄, filtered, concentrated, and purified on silica [30% EtOAc-hexane] to afford 2.75 g of 5 as colorless thick syrup.

Reacted 5 (2.75 g), EtOH (55 mL), NH₂NH₂·H₂O (3 eq) at room temperature 16 hours quenched with ice cold water (30 mL), extracted with EtOAc (2×105 mL), combined organic layer was dried over Na₂SO₄, filtered, and concentrated to afford 2 g of 6 as colorless thick syrup.

Mixed 6 (1.0 g), 1,4-dioxane (20 mL), TEA (3 eq), FNAB (1.0 eq) at room temperature then 80° C. 16 hours. The reaction was quenched with ice cold water (40 mL), extracted with EtOAc (2×70 mL), combined organic layer was dried over Na₂SO₄, filtered, concentrated, and purified on silica [20% EtOAc-hexane] to afford 350 mg of 8 as colorless thick syrup.

Reacted 8 (300 mg), MeOH (10 mL), 4M HCl in 1,4-dioxane (4.0 eq) at 0° C. for 4 hours. Volatiles were concentrated and preparative HPLC purification afforded 10 mg of 1272 as orange-red thick syrup. In a separate deprotection, reacted 8 (100 mg), 1,4-dioxane (2 mL), 4M HCl in 1,4-dioxane (4.0 eq) at 0° C. then at room temperature for 4 hours. The volatiles were concentrated and purification by preparative HPLC afforded 25 mg of 1253.

### Example 97: Synthesis of Compound 1255 ((2R,3R,4R,5S)-1-(2-{2-[(4-azido-2-nitrophenyl)amino]ethoxy}ethyl)-2-(hydroxymethyl)piperidine-3,4,5-triol)

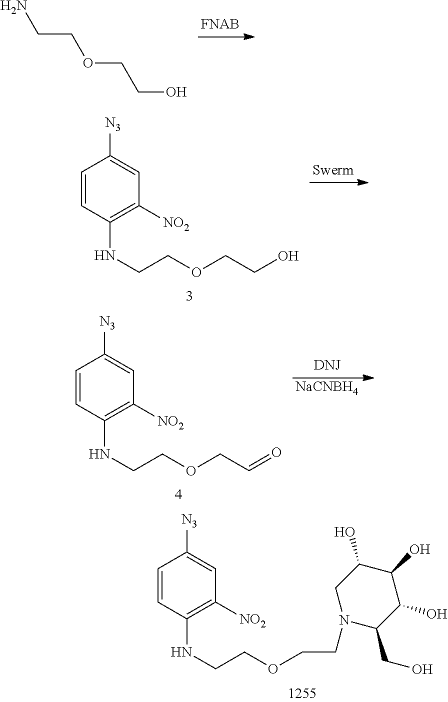

FNAB (2 g), 2-(2-aminoethoxy)ethan-1-ol (1.2 eq), 1,4-dioxane (40 mL), TEA (3.0 eq) reacted at 80° C. 16 hours. Reaction was concentrated, residue was dissolved in EtOAc (50 mL) and washed with water (50 mL). Organic layer was dried over Na₂SO₄, filtered, concentrated, and purified by column [70% EtOAc in hexane] to afford 2.2 g of 3.

(COCl)₂ (2 eq), DCM (10 mL), DMSO (3.6 eq) at −78° C. mixed 0.5 hours added 3 (500 mg) in DCM (3 mL) for 0.5 hours added TEA (5 eq) and raised to room temperature for 1 hour. Quenched with ice-cold water and extracted with DCM (2×20 mL). Organic layer was dried over Na₂SO₄, filtered, and concentrated to afford 300 mg of 4 as orange red thick syrup.

Mixed 4 (300 mg), MeOH (20 mL), DNJ (0.8 eq), AcOH (cat.), NaCNBH₃ (1.5 eq) at room temperature for 16 hours. Concentrated volatiles. Preparative HPLC purification gave 178 mg of 1255.

### Example 98: Synthesis of Compound 1268 (2-[(4-azido-2-nitrophenyl)amino]ethyl N-{2-[(2R,3R,4R,5S)-3,4,5-trihydroxy-2-(hydroxymethyl)piperidin-1-yl]ethyl}carbamate)

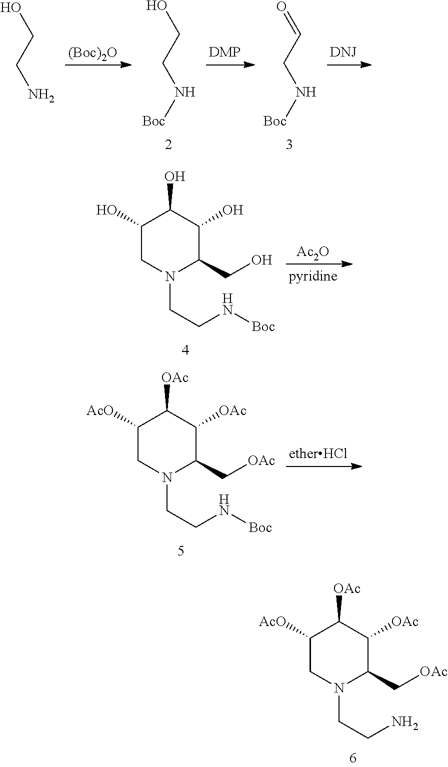

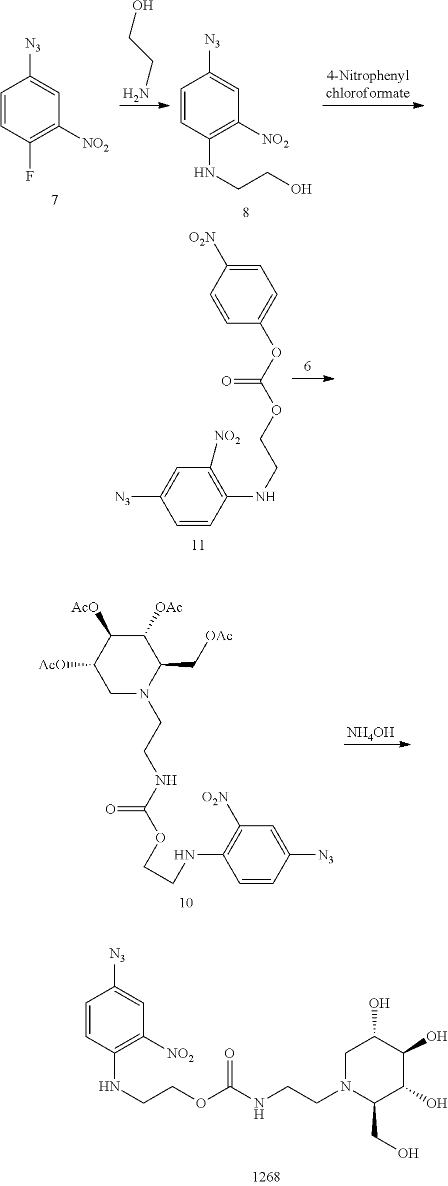

1 (40 g), THF:saturated NaHCO₃ (800 mL, 1:1), (BOc)₂O (1.0 eq) mixed at 0° C. then 16 hours at room temperature. Reaction was extracted with EtOAc (2×200 mL), combined organic layer was washed with water, dried over anhydrous Na₂SO₄, filtered and concentrated to afford 2.

2 (2.0 g), DCM (20 mL), DMP (1.5 eq) mixed at 0° C. then room temperature for 1 hour. Reaction was quenched with aqueous saturated NaHCO₃ solution and extracted with DCM (2×20 mL). Combined organic was washed with water, dried over Na₂SO₄, filtered, concentrated, and purified on silica [30% EtOAc in hexane] to afford 1 g of 3 as pale yellow syrup.

3 (1 g), MeOH (20 mL), DNJ (1 eq), NaCNBH₃ (1.5 eq), AcOH (cat.) reacted at room temperature, 12 hours. The reaction was concentrated and residue purified on silica [15% MeOH in DCM] to afford 1 g of 4 as pale yellow syrup.

4 (1 g), CHCl₃/pyridine (1:1, 30 mL) mixed at 0° C., added Ac₂O (10 eq), reacted at room temperature 48 hours. The reaction was concentrated and residue purified on silica [40% EtOAc in hexane] to afford 1 g of 5 as pale yellow syrup.

Mixed 5 (1 g), DCM (3 mL) at 0° C., added HCl in ether (10 mL), reacted 2 hours at room temperature. The reaction was concentrated and residue triturated with ether/pentane to give 350 mg of 6.

FNAB (3 g) in 1,4-dioxane (60 mL), 2-aminoethan-1-ol (1.2 eq), TEA (3.0 eq) reacted at 100° C., 12 hours. Volatiles were evaporated and the residue was dissolved in EtOAc (2×50 mL), washed with water, the organic layer was dried over Na₂SO₄, filtered, concentrated, and purified on silica [30% EtOAc in hexane] to afford 2.3 g of 8 as red solid.

8 (400 mg), DCM (20 mL) mixed at 0° C., added 4-nitrophenyl chloroformate (1.5 eq), TEA (2 eq), reacted 4 hours at room temperature. Reaction was diluted with ice-cold water and extracted with DCM (2×20 mL). The combined organic layer was washed with water, dried over Na₂SO₄, filtered, concentrated, and re-crystalized with methanol to afford 300 mg of 11.

6 (300 mg), THF (15 mL), 11 (1.2 eq), NaHCO₃ (4.0 eq) reacted in sealed tube at 100° C. for 12 hours. Volatiles were concentrated and material was triturated with DCM/pentane then purified on silica [30% EtOAc in hexane] to afford 300 mg of 10 as red semisolid.

10 (300 mg), MeOH (15 mL) mixed at 0° C., added 25% aq. NH₃ sol. (8 mL), reacted at room temperature in sealed tube 12 hours. Volatiles were evaporated and residue crystallized with MeOH-DCM (2:10, 30 mL), washed with pentane, then lyophilized to give 150 mg 1268.

### Example 99: Synthesis of Compound 1276 (1-{2-[(4-azido-2-nitrophenyl)amino]ethyl}-3-{2-[(2R,3R,4R,5S)-3,4,5-trihydroxy-2-(hydroxymethyl)piperidin-1-yl]ethyl}urea)

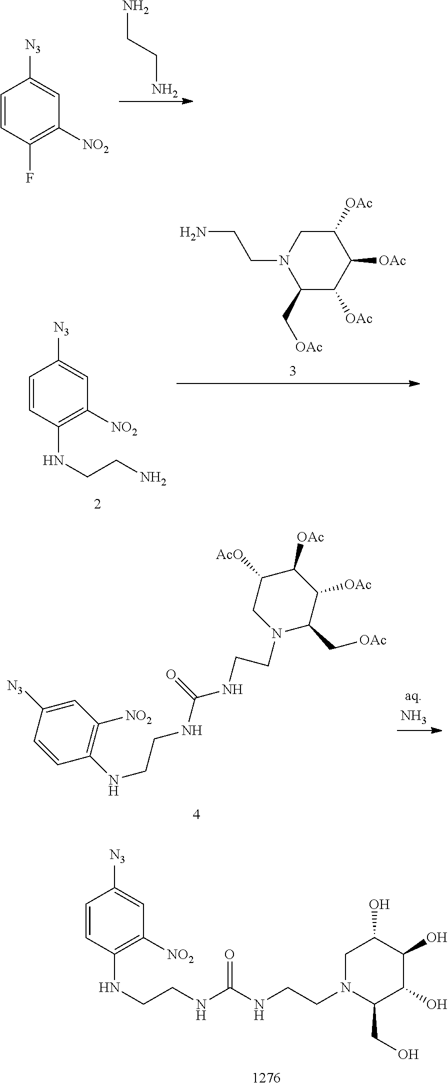

FNAB (3 g), CHCl₃ (60 mL), 1,2-diaminoethane (1.5 eq), TEA (3.0 eq) refluxed 12 hours. Reaction was diluted with ice-cold water, extracted with DCM (2×50 mL), combined organic layer was washed with water and dried over Na₂SO₄, filtered, concentrated, and purified on silica [10% MeOH in DCM] to afford 2.3 g of 2 as red solid.

3 (400 mg, Example 98), DCM (20 mL), triphosgene (1 eq), TEA (15 eq) mixed at 40° C., 60 minutes, added 2 (1 eq), reacted 12 hours at room temperature. Reaction was diluted with ice-cold water, extracted with DCM (2×15 mL), combined organic layer was dried over Na₂SO₄, filtered, concentrated, and purified by column [3% MeOH-DCM] to give 300 mg of 4 as red solid.

4 (300 mg), MeOH (15 mL) mixed at 0° C., added 25% aq. NH₃ sol. (10 mL), reacted at room temperature in sealed tube 12 hours. Volatiles were concentrated and the product crystallized with MeOH:DCM (2:10, 20 mL) then washed with pentane followed by lyophilization to afford 80 mg of 1276.

### Example 100: Synthesis of Compound 1274 ((2R,3R,4R,5S)-1-(3-{2-[(4-azido-2-nitrophenyl)amino]ethane-sulfonyl}propyl)-2-(hydroxymethyl)piperidine-3,4,5-triol)

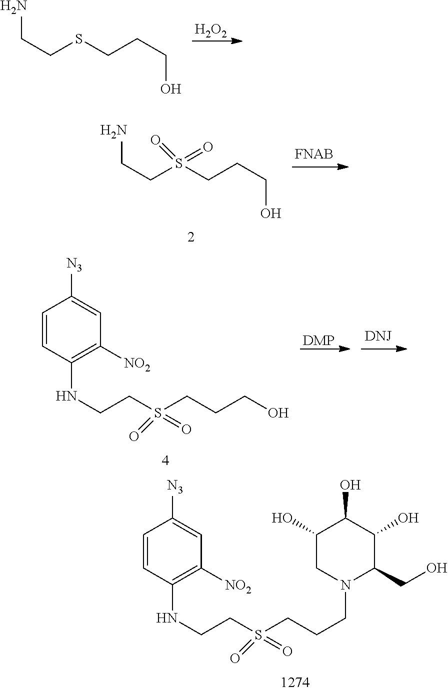

3-((2-aminoethyl)thio)propan-1-ol (1 g), AcOH (20 mL), H₂O (20 mL), H₂O₂ (3.0 eq) mixed at room temperature then at 50° C. for 6 hours. The reaction was concentrated to afford 2 (1.1 g) as colorless thick syrup.

2 (1.1 g), FNAB (0.8 eq), TEA (0.5 mL), 1,4-dioxane (25 mL) mixed at room temperature then at 80° C. 8 hours. The reaction solvents were evaporated, residue was quenched with water and extracted with EtOAc (2×100 mL), combined organic layer was washed with water, dried over anhydrous Na₂SO₄, filtered, concentrated, and purified on silica [5% MeOH-DCM] to afford 700 mg of 4.

4 (500 mg), DMP (1.5 eq), DCM (25 mL), 0° C.-room temperature, 16 hours. The reaction mass was quenched with saturated NaHCO₃, extracted with DCM (2×30 mL), combined organic layer was washed with water, dried over anhydrous Na₂SO₄, filtered and concentrated. This afforded 500 mg of the aldehyde which was used immediately in the subsequent coupling with MeOH (20 mL), DNJ (0.8 eq), AcOH (cat), NaCNBH₃ (1.5 eq) reacted at room temperature for 16 hours. Volatiles were evaporated and preparative HPLC purification afforded 110 mg of 1274 as yellow solid.

### Example 101: Synthesis of Compound 1277 ((2R,3R,4R,5S)-1-{8-[(4-amino-3-chloro-2-nitrophenyl)amino]octyl} 2-(hydroxymethyl)piperidine-3,4,5-triol)

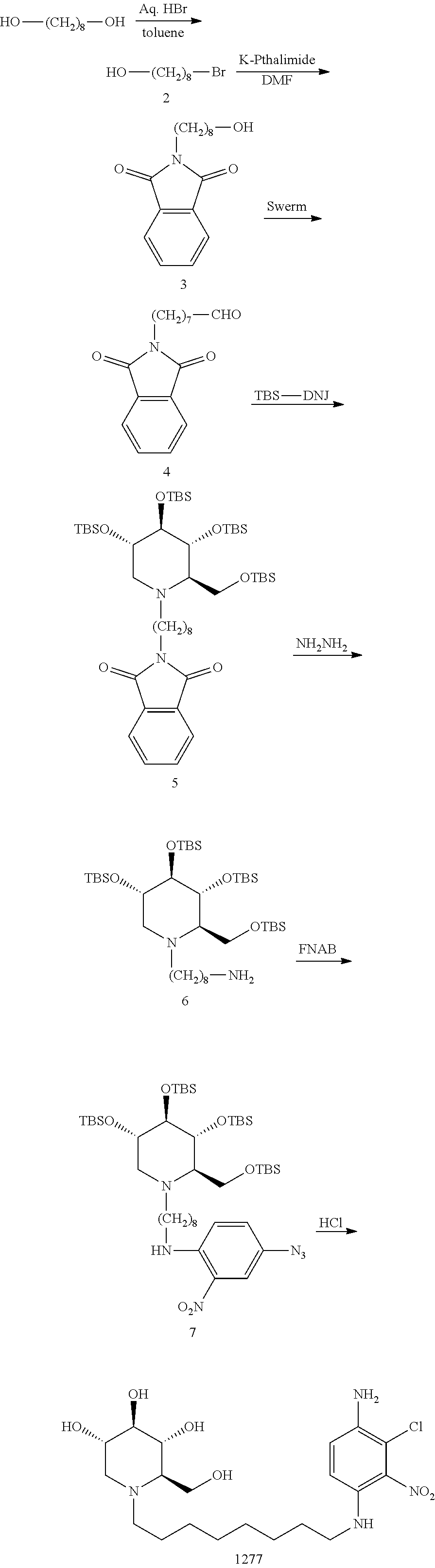

1,8-Dihydroxyoctane (10 g), toluene (100 mL), 47% aqueous HBr (70 mL) mixed at room temperature then raised to 100° C. After 8 hours, the reaction was diluted with ice-water, extracted with EtOAc (2×100 mL), combined organic layer was washed with water, dried over anhydrous Na₂SO₄, filtered, concentrated, and purified on silica [20% EtOAc-hexane] to afford 13 g of 2 as color-less thick syrup.

2 (2.0 g), DMF (20 mL), K-phthalimide (1.5 eq) mixed at room temperature then at 150° C. 4 hours. Reaction was diluted with ice-cold water, extracted with EtOAc (2×20 mL), organic layer was washed with water, dried over Na₂SO₄, filtered, concentrated, and purified on silica with 30% EtOAc-hexane to afford 2.0 g of 3 as colorless thick syrup.

Oxalylchloride (2.0 eq), THF (5.0 mL), DMSO (4.0 eq) mixed at −78° C. 10 minutes, added 3 (1.5 g), mixed at −78° C. 20 minutes, added TEA (4.0 eq) at −78° C. 1 hour, increased to room temperature for 3 hours. Reaction was diluted with ice-cold water, extracted with EtOAc (2×20 mL), combined organic layer washed with water, dried over Na₂SO₄, filtered, concentrated, and purified on silica with 20% EtOAc-hexane to afford 1.2 g of 4 as colorless thick syrup.

4 (1.2 g), MeOH (25 mL), TBS-DNJ (0.8 eq), AcOH (cat), NaCNBH₃ (1.5 eq) reacted at room temperature 16 hours. Volatiles were removed, residue was diluted with ice-cold water and extracted with EtOAc (2×20 mL), combined organic layer was washed with water, dried over Na₂SO₄, filtered, concentrated, and purified on silica with 10% EtOAc-hexane to afford 800 mg of 5 as colorless thick syrup.

5 (800 mg), EtOH (15 mL), NH₂NH₂·H₂O (5.0 eq) reacted at room temperature 16 hours. Reaction was diluted with ice-water, extracted with EtOAc (2×30 mL), organic layer was washed with water and dried over anhydrous Na₂SO₄, filtered, and concentrated to afford 600 mg of amine 6 as colorless thick syrup.

6 (600 mg), 1,4-dioxane (20 mL), FNAB (0.8 eq), and TEA (3.0 eq) were mixed at room temperature, temperature increased to 80° C. After 16 hours reaction was diluted with ice-cold water and extracted with EtOAc (2×20 mL). The combined organic layer was washed with water, dried over Na₂SO₄, filtered, concentrated, and purified on silica [10% EtOAc-hexane] to afford 600 mg of 7 as orange red thick syrup.

Mixed 7 (550 mg), 1,4-dioxane (10 mL), 4 M HCl in 1,4-dioxane (4.0 mL) at 0° C. then 16 hours at room temperature. Removed volatiles, preparative HPLC purification gave 20 mg 1277.

### Example 102: Synthesis of Compound 1280 ((2R,3R,4R,5S)-1-[(5-{[(4-azido-2-nitrophenyl)amino]methyl}-1,4-dioxan-2-yl)methyl]-2-(hydroxymethyl)piperidine-3,4,5-triol)

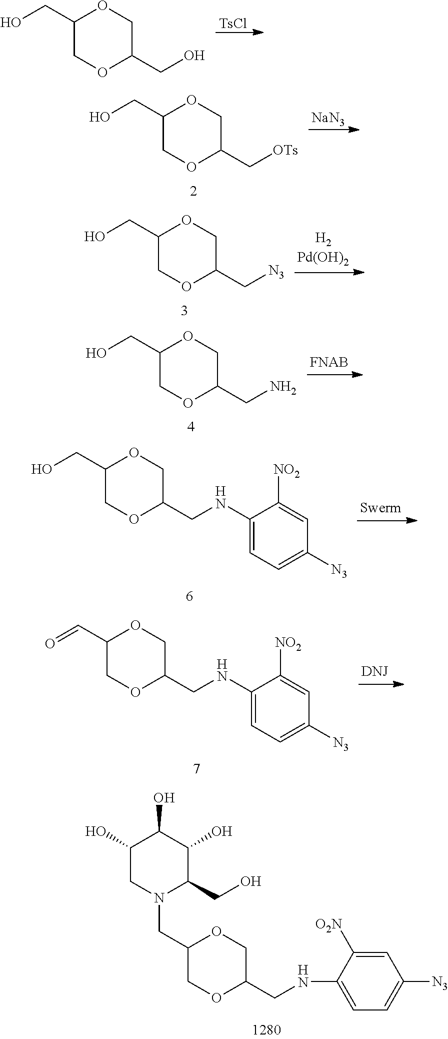

(1,4-dioxane-2,5-diyl)dimethanol (600 mg), DCM (12 mL), TsCl (1 eq), TEA (2 eq) reacted at room temperature 12 hours. The reaction mass was quenched with 3.0 M aq. HCl, the organic layer was separated and dried over Na₂SO₄ and concentrated to obtain 520 mg of 2.

2 (520 mg), DMSO (10 mL), NaN₃ (5.0 eq) reacted at 80° C., 8 hours. The reaction mass was cooled to room temperature, quenched with ice-cold water (20 mL), extracted with EtOAc (2×15 mL), the organic layer washed with water (20 mL), dried over Na₂SO₄, filtered and concentrated to afford 250 mg of 3.

3 (250 mg), EtOH (10 mL), Pd(OH)₂ (50 mg) reacted under H₂ at room temperature 8 hours. Reaction was filtered through CELITE bed under N₂. Filtrate was concentrated to afford 120 mg of 4.

4 (120 mg), FNAB (1 eq), TEA (5 eq) reacted at 80° C., 16 hours. Reaction was concentrated then purified by column [50% EtOAc-hexane] to afford 100 mg of 6 as red solid.

(COCl)₂ (2 eq), DCM (0.5 mL), DMSO (4 eq), 6 (100 mg) mixed at −78° C. 2 hours then TEA (6 eq) added and bought to room temperature. Reaction was quenched with ice-cold water, extracted with DCM (10 mL), the organic dried over Na₂SO₄, filtered and concentrated to afford 7.

7 (90 mg), MeOH (10 mL), DNJ (1 eq), AcOH, NaCNBH₃ (1.5 eq) reacted at room temperature, 12 hours. The reaction was concentrated, combined with another smaller batch, and preparative HPLC purification afforded 23 mg of 1280.

### Example 103: Synthesis of Compounds 1297 (5-azido-2-({6-[(2R,3R,4R,5S)-3,4,5-trihydroxy-2-(hydroxymethyl)piperidin-1-yl]hexyl}amino)benzoic acid) and 1288 (methyl 5-azido-2-({6-[(2R,3R,4R,5S)-3,4,5-trihydroxy-2-(hydroxymethyl)piperidin-1-yl]hexyl}amino)benzoate)

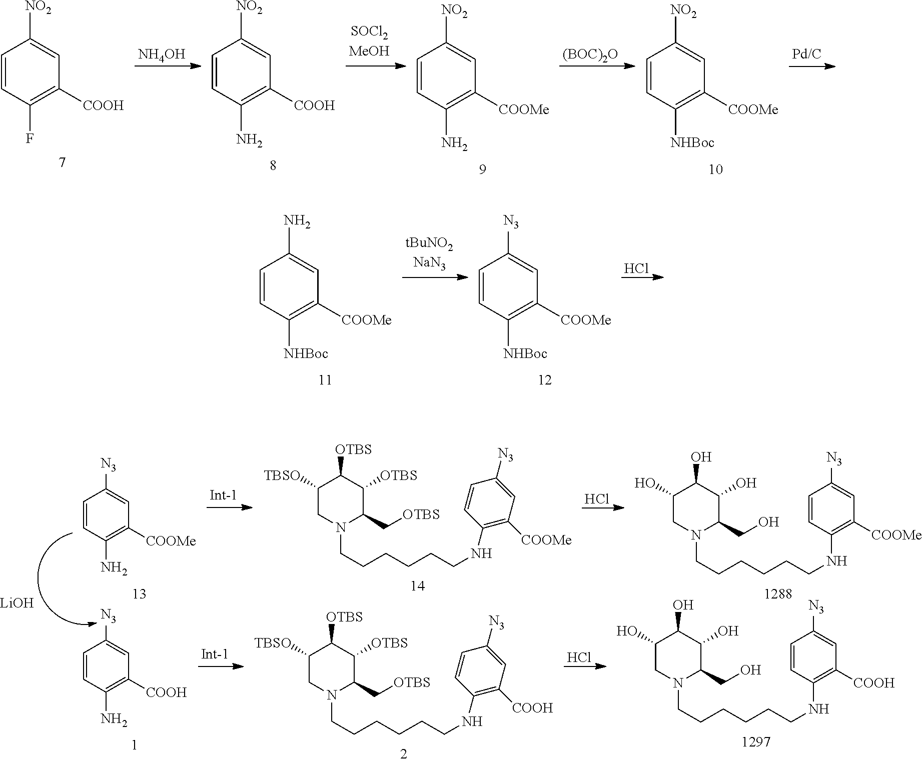

Stirred a solution of 2-fluoro-5-nitrobenzoic acid (500 mg) in 1,4-dioxane (10 mL) in a sealed tube, added NH₄OH (10 mL) at room temperature, stirred at 60° C., 4 hours. Volatiles were concentrated and product was purified by column [50% EtOAc/hexane] to afford 520 mg of 8 as yellow colored solid. Similarly, 15.0 g of 7 gave 11.0 g of 8.

8 (500 mg) in MeOH (10 mL), added SOCl₂ (5.0 eq) at 0° C., warmed to 90° C. for 72 hours. Volatiles were concentrated, residue quenched with ice-cold water, extracted with EtOAc (2×30 mL), organic layers were washed with saturated NaHCO₃ and brine, dried over anhydrous Na₂SO₄, filtered and concentrated to afford 400 mg of 9 as yellow solid. Similarly, 11.0 g of 8 gave 8.0 g of 9.

9 (380 mg), DCM (10 mL), TEA (2.0 eq) stirred 20 minutes at room temperature, added (Boc)₂O (1.2 eq), DMAP (cat.) at room temperature, stirred 4 hours. Reaction was quenched with ice-cold water, extracted with DCM (2×20 mL), combined organic layers were washed with brine, dried over anhydrous Na₂SO₄, filtered, concentrated, purified on silica [10% EtOAc/hexane] to afford 300 mg of 10 as yellow solid. Similarly, 8.0 g of 9 gave 8.2 g of 10.

10 (300 mg), MeOH:EtOAc (1:1, 10 mL), added Pd/C (200 mg) at room temperature, stirred under H₂ (balloon pressure) 4 hours. Reaction mass was filtered through a CELITE bed, washed with MeOH, filtrate was concentrated to afford 300 mg of 11 as white solid. Similarly, 8.2 g of 10 gave 7.5 g of 11.

11 (300 mg) in ACN (10 mL), added tBuNO₂ (5.0 eq) at 0° C., stirred 10 minutes, added TMS-N₃ (3.0 eq) at 0° C., stirred at room temperature 4 hours. Reaction mass was quenched with ice-cold water, extracted with EtOAc (2×20 mL), organic layers were washed with brine, dried over anhydrous Na₂SO₄, filtered, concentrated, purified on silica [10% EtOAc/hexane] to afford 200 mg of 12 as yellow solid. Similarly, 7.5 g of 11 gave 6.0 g of 12.

12 (100 mg), DCM (5 mL), 4M HCl in 1,4-dioxane (0.2 mL) at 0° C., warmed to room temperature, stirred 16 hours, added 4 M HCl in 1,4-dioxane (0.5 mL) to complete the reaction. Reaction mass was quenched with saturated NaHCO₃, extracted with DCM (2×20 mL), organic layers washed with brine, dried over anhydrous Na₂SO₄, filtered and concentrated to afford 60 mg of 13 as white solid.

Preparation of 1288 methyl 5-azido-2-({6-[(2R,3R,4R,5S)-3,4,5-trihydroxy-2-(hydroxymethyl)piperidin-1-yl]hexyl}amino)benzoate: 13 (65 mg), MeOH (5 mL), Int-1 (1.0 eq), AcOH (cat.) stirred at room temperature 10 minutes, added NaCNBH₃ (1.5 eq), stirred at room temperature 16 hours. Volatiles were concentrated and the material was purified on silica [5% EtOAc/hexane] to afford 65 mg of 14 as colorless thick syrup.

14 (65 mg), 1,4 dioxane (3 mL), 4M HCl in 1,4 dioxane (0.2 mL) mixed at 0° C., stirred at room temperature for 16 hours, added HCl:1,4 dioxane (0.4 mL) and continued at room temperature for 16 hours. After mass-based preparative HPLC purification using formic acid mobile phase, lyophilized to afford 6 mg of 1288 as brown thick syrup.

Preparation of 1297 5-azido-2-({6-[(2R,3R,4R,5S)-3,4,5-trihydroxy-2-(hydroxymethyl)piperidin-1-yl]hexyl}amino)benzoic acid: To a stirred solution of 13 (500 mg) in THF:H₂O (1:1, 10 mL) added LiOH (3.0 eq) at room temperature, stirred 16 hours quenched with 1N HCl (pH 5 to 6) at 0° C., extracted with EtOAc (2×30 mL), combined organic layer was washed with brine solution, dried over anhydrous Na₂SO₄, filtered and concentrated to afford 320 mg of 1 as white solid.

To a stirred solution of 1 (140 mg) in MeOH (10 mL) added 13 (1.0 eq), AcOH (cat.) at room temperature, stirred for 10 minutes, added NaCNBH₃ (1.5 eq), stirred at room temperature 16 hours. Volatiles were concentrated, the residue was diluted with ice-cold water and extracted with EtOAc (2×10 mL), the combined organic layer was dried over anhydrous Na₂SO₄ filtered and concentrated and the material purified by column chromatography [50% EtOAc in hexane] to afford 450 mg of 2 as colourless thick syrup.

To a stirred solution of 2 (450 mg) in 1,4-dioxane (10 mL) added 4M HCl in 1,4-dioxane (1 mL) at 0° C., warmed to room temperature and stirred for 16 hours. Added additional 4M HCl in 1,4-dioxane (1 mL) at 0° C., warmed to room temperature and stirred for 16 hours. Volatiles were concentrated and preparative HPLC purification yielded 40 mg of 1297.

### Example 104: Synthesis of Compound 1296 (5-azido-2-({6-[(2R,3R,4R,5S)-3,4,5-trihydroxy-2-(hydroxymethyl)piperidin-1-yl]hexyl}amino)benzamide)

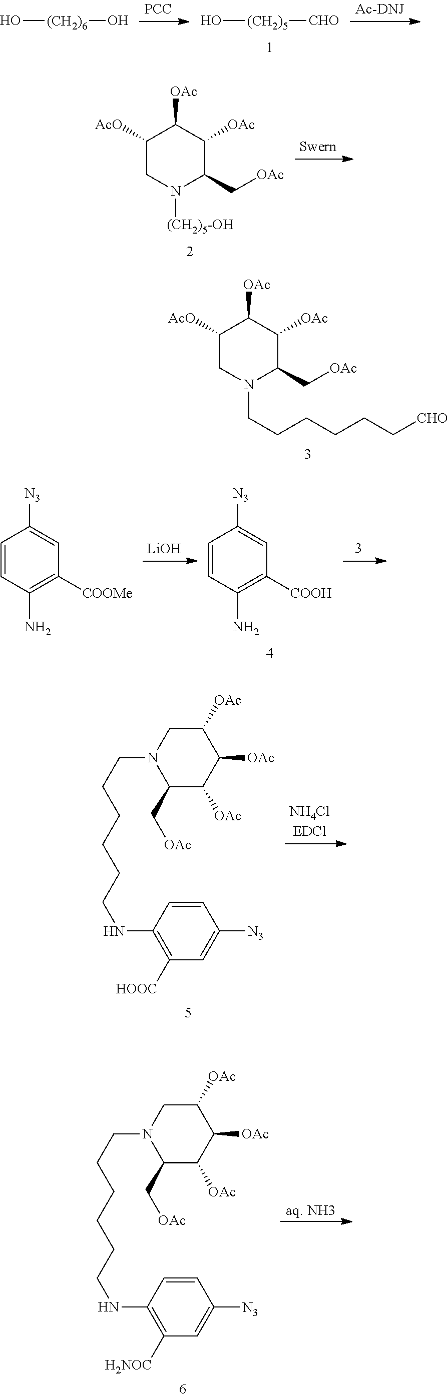

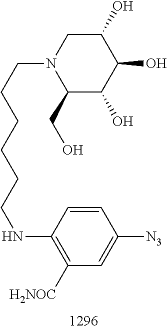 [SMILES: [N-]=[N+]=Nc1ccc(NCCCCCCN2C[C@H](O)[C@@H](O)[C@H](O)[C@H]2CO)c(C(N)=O)c1]

Hexane-1,6-diol (30.0 g), CHCl₃ (600 mL), PCC (1.5 eq) mixed at 10° C. than 2 hours at room temperature. The reaction was filtered through a CELITE bed and washed with DCM (500 mL). Filtrate was concentrated and purified on silica [50% EtOAc-hexane] to give 7.0 g of 1.

1 (4.2 g), Ac-DNJ (1.0 eq, Example 1), MeOH (200 mL), AcOH (cat.) stirred 10 minutes at room temperature, added NaCNBH₃ (1.5 eq) mixed at room temperature 2 hours. Volatiles were removed and residue purified on silica [5% EtOAc:hexane] to afford 7.0 g of 2.

Oxalylchloride (2.1 eq), dry THE (40 mL), DMSO (2.6 eq) mixed at −78° C. for 30 minutes, added 2 (3.0 g in 10 mL dry THF), mixed −78° C. 2 hours then added TEA (5.4 eq) at −78° C. and maintained 2 hours at the same temperature. Reaction was quenched with ice-cold water and extracted with DCM (2×30 mL). Organic extracts were washed with brine solution, dried over anhydrous Na₂SO₄, concentrated, and purified by silica gel flash column [5% EtOAc/hexane] to afford 2.0 g of 3 as colorless thick syrup.

2-amino-5-azidobenzoic acid methyl ester (900 mg, from Example 103) in THF:H₂O (15 mL, 1:1) added LiOH (3.0 eq) at room temperature and stirred for 16 hours. Volatiles were concentrated and residue was diluted with ice-cold water (10 mL) and acidified with 1N HCl (pH 5 to 6) and extracted with EtOAc (2×20 mL). Combined organic extract was dried over anhydrous Na₂SO₄, filtered and concentrated to afford 600 mg of 4 as off-white solid.

To a stirred solution of 4 (1 g), and 4 (1.0 eq) in MeOH (30 mL) added AcOH (cat.) at room temperature, stirred for 1 hour added NaCNBH₃ (1.5 eq), stirred 16 hours at room temperature. Volatiles were concentrated and the material was purified on silica [20% EtOAc/hexane] to afford 400 mg of 5 as colorless thick syrup.

To a stirred solution of compound 5 (100 mg) in DMF (3 mL) and DIPEA (3 eq) added NH₄Cl (3 eq), 1-ethyl-3-(3-dimethylaminopropyl)carbodiimide (3.0 eq), and hydroxy-benzotriazole (2.0 eq) at room temperature and stirred 16 hours. Reaction mass was diluted with ice-cold water (10 mL) and extracted with EtOAc (2×10 mL). Combined organic layer was washed with water (2×10 mL), dried over anhydrous Na₂SO₄, filtered, concentrated, and purified on silica [30% EtOAc/hexane] to afford 80 mg of 6 as colorless thick syrup.

To a stirred solution of 6 (80 mg) in EtOH (3 mL) added EtOH·NH₃ (2 mL) in a sealed tube at room temperature and reacted 16 hours. Volatiles were concentrated and the material purified by preparative HPLC using trifluoroacetic acid as a mobile phase modifier to afford 25 mg of 1296 as TFA salt as colorless thick syrup.

### Example 105: Synthesis of Compound 1290 ((2R,3R,4R,5S)-2-(hydroxymethyl)-1-(6-{[2-nitro-4-(prop-1-yn-1-yl)phenyl]amino}hexyl)piperidine-3,4,5-triol)

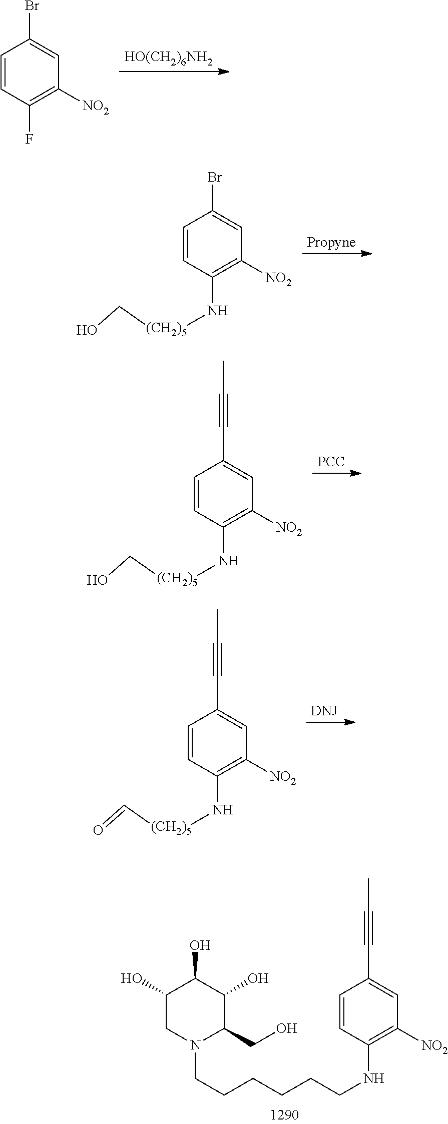

To a stirred solution of 1-fluoro-4-bromo-2-nitrobenzene (5 g) in 1,4-dioxane (30 mL) and TEA (5.0 eq) added 6-aminohexanol (1.5 eq) at room temperature, stirred at 60° C. 16 hours. Volatiles were concentrated and the material purified on silica [40% EtOAc/hexane] to obtain 6 g of 2 as yellow solid.

To a stirred solution of 2 (1 g) in TEA (10 mL) added Pd(PPh₃)₂Cl₂ (0.1 eq), CuI (0.1 eq), and propyne gas (10 psi) in a 100 mL steel vessel at room temperature, stirred 50° C. 16 hours. Concentrated volatiles, diluted residue with ice-cold water (20 mL), extracted with EtOAc (2×30 mL). Combined organic extract was dried over anhydrous Na₂SO₄, filtered, concentrated, and purified on silica [20% EtOAc/hexane] to afford 650 mg of 3 as yellow solid.

To a stirred solution of 3 (400 mg) in CHCl₃ (20 mL) added PCC (1.5 eq) at room temperature and stirred 4 hours. Reaction mass was filtered through a CELITE bed, washed with DCM (20 mL), filtrate was concentrated and the material was purified on silica [10-20% EtOAc/hexane] to afford 200 mg of 4 as yellow thick syrup.

To a stirred solution of 4 (200 mg) in MeOH (10 mL) added DNJ (0.8 eq), AcOH (cat.), and NaCNBH₃ (1.5 eq) at room temperature, stirred for 16 hours. Volatiles were concentrated and material was purified by preparative HPLC to afford 50 mg of 1290 as yellow solid.

### Example 106: Synthesis of Compounds 1291 (propyl 2-[3-nitro-4-({6-[(2R,3R,4R,5S)-3,4,5-trihydroxy-2-(hydroxymethyl)piperidin-1-yl]hexyl}amino)phenyl]acetate (and 1292 ((2R,3R,4R,5S)-1-(6-{[4-(2-hydroxyethyl)-2-nitrophenyl]amino}hexyl)-2-(hydroxymethyl)piperidine-3,4,5-triol)

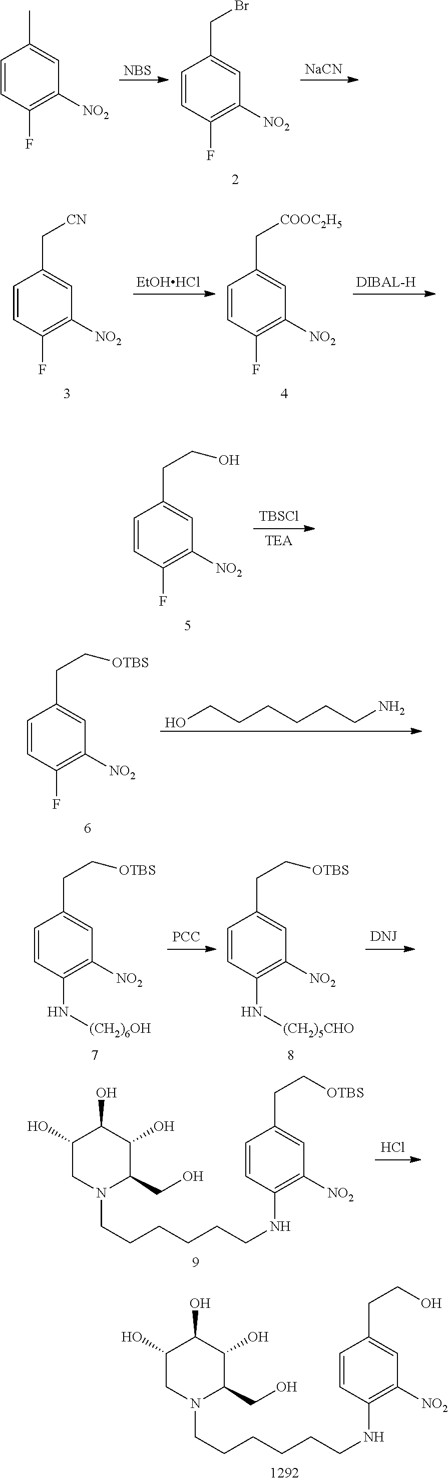

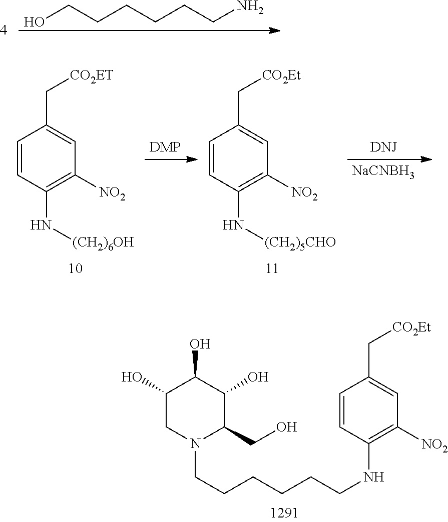

To a stirred solution of 3-nitro-4-fluorotoluene (10 g) in CCl₄ (300 mL) added NBS (1.2 eq) and benzoyl peroxide (0.1 eq) at 10° C., warmed to 80° C., maintained 16 hours. Reaction mass was quenched with ice-cold water and extracted with DCM (2×100 mL). Combined organic extract was washed with brine, dried over anhydrous Na₂SO₄ and concentrated to afford 4.5 g of 2 as white solid.

To a stirred solution of 2 (4 g) in EtOH (50 mL) added NaCN (1.1 eq) at room temperature, warmed to 45° C., maintained for 16 hours. The volatiles were removed, residue was diluted with ice-cold water and extracted with EtOAc (2×100 mL), organic layers were dried over anhydrous Na₂SO₄, filtered, concentrated, and purified on silica [20% EtOAc-hexane] to afford 1.6 g of 3 as oily liquid.

Stirred a solution of 3 (1.6 g), EtOH (5 mL) and ethanolic HCl (20 mL) at room temperature for 16 hours. Volatiles were removed, residue was diluted with ice-cold water, extracted with EtOAc (2×30 mL), organic extracts were dried over anhydrous Na₂SO₄, filtered, concentrated, and purified on silica [30% EtOAc-hexane] to afford 1.5 g of 4 as yellow solid.

Preparation of 1292 (2R,3R,4R,5S)-1-(6-{[4-(2-hydroxyethyl)-2-nitrophenyl]amino}hexyl)-2-(hydroxymethyl)piperidine-3,4,5-triol: 4 (1.5 g), THE (20 mL), 1% DIBAL-H in toluene (2.0 eq) mixed at −40° C. for 20 minutes then at room temperature for 5 hours. Reaction mass was quenched with ice-cold water, extracted with DCM (2×30 mL), extracts were washed with brine, dried over anhydrous Na₂SO₄, filtered, concentrated, and purified on silica [40% EtOAc-hexane] to afford 1.0 g of 5 as yellow solid.

5 (500 mg), DCM (10 mL), added TEA (3.0 eq) at room temperature, stirred 20 minutes, TBDMS-Cl (1.2 eq) added at 0° C., warmed to room temperature, stirred 16 hours. Reaction mass was quenched with ice-cold water, extracted with DCM (2×20 mL), extracts were washed with brine, dried over anhydrous Na₂SO₄, concentrated, and purified on silica [10% EtOAc/hexane] to obtain 500 mg of 6 as colorless oily liquid.

6 (500 mg), 1,4-dioxane (10 mL), TEA (5.0 eq), added 6-aminohexanol (1.5 eq) at room temperature, heated to 60° C., maintained 16 hours. Volatiles were removed and material was purified on silica [40% EtOAc/hexane] to afford 500 mg of 7 as yellow solid.

7 (500 mg) in CHCl₃ (30 mL), added PCC (1.5 eq) at room temperature, stirred 4 hours. Reaction was filtered through a CELITE bed, washed with DCM (30 mL), filtrate was concentrated and purified on silica [10-20% EtOAc/hexane] to afford 250 mg of 8 as yellow thick syrup.

To a stirred solution of 8 (250 mg) in MeOH (10 mL) added DNJ (0.8 eq), AcOH (cat.) and NaCNBH₃ (1.5 eq) at room temperature stirred 16 hours. Volatiles were removed and the material was purified on silica [5-10% MeOH/DCM] to afford 200 mg of 9 as yellow thick syrup.

To a stirred solution of 9 (200 mg) in 1,4-dioxane (4 mL) added 4.0 M HCl in 1,4-dioxane (2 mL) at 0° C., warmed to room temperature and maintained 16 hours. Volatiles were concentrated and residue triturated with Et₂O (2×20 mL) and dried to afford 70 mg of 1292 as yellow solid.

Preparation of 1291 propyl 2-[3-nitro-4-({6-[(2R,3R,4R,5S)-3,4,5-trihydroxy-2-(hydroxymethyl)piperidin-1-yl]hexyl}amino)phenyl]acetate: Stirred 4 (1 g) in 1,4-dioxane (20 mL) and TEA (3.0 eq), added 6-aminohexanol (1.5 eq) at room temperature, warmed to 60° C. and maintained for 16 hours. Volatiles were concentrated and residue purified on silica [30% EtOAc/hexane] to afford 600 mg of 10 as yellow solid.

Mixed 10 (600 mg) in CHCl₃ (20 mL), added PCC (2.5 eq) at room temperature and continued for 4 hours. Reaction was filtered through CELITE bed and washed with DCM (20 mL). The filtrate was concentrated and the material was purified on silica [10-20% EtOAc/hexane] to afford 400 mg of 11 as yellow thick syrup.

Stirred 11 (400 mg) in MeOH (15 mL), added DNJ (0.8 eq), AcOH (cat,) and NaCNBH₃ (1.5 eq) at room temperature and continued for 16 hours. Volatiles were concentrated and residue purified on silica [7-10% MeOH/DCM] to afford 380 mg of 1291 as yellow solid.

### Example 107. Synthesis of Compounds 1310 ((2R,3R,4R,5S)-2-(hydroxymethyl)-1-[5-(4-methoxyphenyl)pentyl]piperidine-3,4,5-triol), 1309 ((2R,3R,4R,5S)-2-(hydroxymethyl)-1-[7-(4-methoxyphenyl)heptyl]piperidine-3,4,5-triol), and 1338 ((2R,3R,4R,5S)-2-(hydroxymethyl)-1-[7-(4-methoxyphenyl)hept-6-yn-1-yl]piperidine-3,4,5-triol)

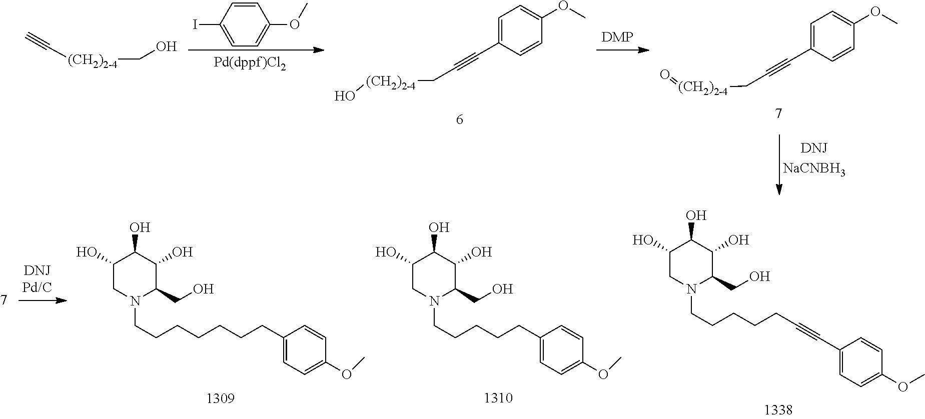

Preparation of 1310 (2R,3R,4R,5S)-2-(hydroxymethyl)-1-[5-(4-methoxyphenyl)pentyl]piperidine-3,4,5-triol: A stirred solution of 1-iodo-4-methoxybenzene (13 g) in ACN (130 mL) was purged with Ar 30 minutes, added TEA (23.5 mL), pent-4-yn-1-ol (4.7 g), CuI (1.0 g) and Pd(dppf)Cl₂ (2.0 g) purged with Ar 10 minutes, stirred for 16 hours at room temperature. Mixture was diluted with ice-cold water and extracted with EtOAc (2×200 mL). Combined organic extracts were washed with brine, dried over anhydrous Na₂SO₄, concentrated, and purified on silica with 10% EtOAc/hexane to afford 8.0 g of 6 as thick syrup.

To a stirred solution of 6 (3.0 g) in DCM (50 mL) added DMP (8.0 g) at 0° C., stirred for 3 hours at room temperature. Quenched with saturated NaHCO₃ solution (50 mL), extracted with DCM (2×50 mL), organic extracts were dried over anhydrous Na₂SO₄, concentrated, and purified on silica with 7-8% EtOAc/hexane to afford 2.4 g of 7 as colorless thick syrup.

7 (2.4 g), MeOH (75 mL), DNJ (1.7 g), AcOH (cat.) stirred 10 minutes at room temperature, added Pd/C (2.5 g), stirred 16 hours at room temperature under H₂ (balloon pressure). Mixture was filtered through CELITE bed, washed with MeOH (100 mL), the filtrate was concentrated and purified on silica with 8-10% MeOH/CH₂Cl₂ to afford 2.2 g of 1310 as white solid.

Preparation of 1309 (2R,3R,4R,5S)-2-(hydroxymethyl)-1-[7-(4-methoxyphenyl)heptyl]piperidine-3,4,5-triol: To prepare the heptyne starting material shown in the figure, to a stirred solution of prop-2-yn-1-ol (20 g) in THE (300 mL) added HMPA (150 mL), maintained at 78° C. while adding n-BuLi (489 mL, 2.2 eq), warmed to −30° C., added n-butylbromide (46.1 mL), stirred at room temperature for 16 hours. Quenched with saturated NH₄Cl solution and extracted with Et₂O (2×100 mL), combined organic extracts were washed with water and dried over anhydrous Na₂SO₄, filtered, concentrated, and purified on silica with 30% EtOAc-hexane to afford hept-2-yn-1-ol (15 g) as colorless thick syrup. A solution of NaH (2.57 g) in hexane (40 mL) was stirred for 5 minutes and the solvent was decanted, added 1,3 diaminopropane (60 mL) at 0° C., stirred at 80° C. for 1 hour. Cooled to room temperature and added hept-2-yn-1-ol (3 g) dissolved in 1,3 diamino-propane, heated at 80° C. 3 hours. Quenched with ice-cold water and extracted with DCM (2×30 mL), combined organic layers were washed with 1N HCl (20 mL) and water (30 mL) and dried over anhydrous Na₂SO₄, filtered, and concentrated to afford the indicated hept-6-yn-1-ol (1.6 g) as thick syrup.

To a stirred solution of hept-6-yn-1-ol (5.0 g) in TEA (10 mL), added 1-iodo-4-methoxybenzene (12.5 g), CuI (847 mg), purging 30 minutes with Ar, added Pd(dppf)Cl₂ (3.26 g) at room temperature, degassed with Ar 30 minutes, heated in a sealed tube at 80° C. for 16 hours. Reaction was diluted with ice-cold water and extracted with EtOAc (2×100 mL). Combined organic extracts were washed with brine, dried over anhydrous Na₂SO₄, concentrated, and purified on silica [30% EtOAc/hexane] to afford 5.0 g of 6 as yellow thick syrup.

6 (2.8 g), DCM (60 mL), added DMP (6.5 g) at 0° C., stirred at room temperature for 3 hours. Quenched with saturated NaHCO₃ solution, extracted with DCM (2×50 mL). Combined organic extracts were washed with brine, dried over anhydrous Na₂SO₄, concentrated, and purified on silica flash column with 8-10% EtOAc/hexane to afford 2.4 g of 7 as thick syrup.

7 (2.0 g), MeOH (60 mL), added DNJ (1.21 g), AcOH (cat.) at room temperature, stirred 10 minutes, added Pd/C (2.0 g), stirred at room temperature under H₂ (balloon pressure) for 16 hours, filtered through CELITE bed, washed with MeOH (50 mL), filtrate was concentrated and purified on silica flash column with 8-10% MeOH/CH₂Cl₂ to afford 1.2 g of 1309 as low melting solid.

Preparation of 1338 (2R,3R,4R,5S)-2-(hydroxymethyl)-1-[7-(4-methoxyphenyl)hept-6-yn-1-yl]piperidine-3,4,5-triol: To a stirred solution of 7 (400 mg) in MeOH (10 mL) was added DNJ (242 mg), AcOH (cat.) at room temperature and stirred 10 minutes, added NaCNBH₃ (175 mg), stirred at room temperature for 16 hours. Volatiles were concentrated and material was purified on silica flash column with 8-10% MeOH/EtOAc to afford 200 mg of 1338 as low melting solid.

### Example 108: Synthesis of Compound 1299 (2-[3-nitro-4-({6-[(2R,3R,4R,5S)-3,4,5-trihydroxy-2-(hydroxymethyl)piperidin-1-yl]hexyl}amino)phenyl]acetonitrile)

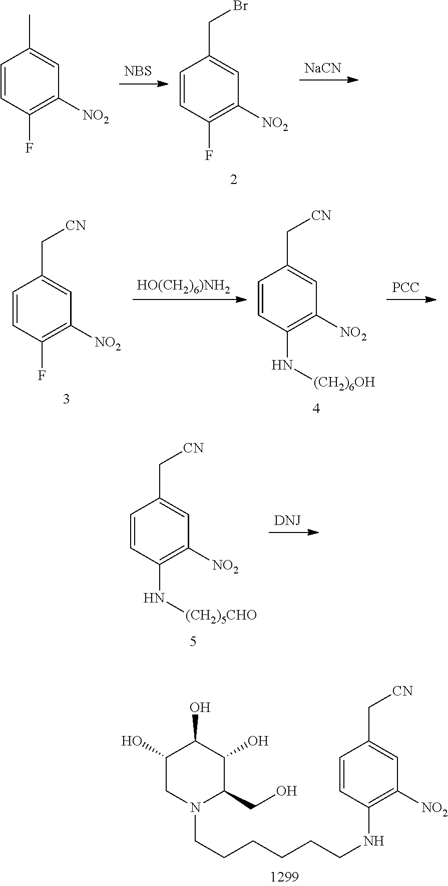

To a stirred solution of 3-nitro-4-fluorotoluene (1.0 g) in CCl₄ (30 mL), added NBS (1.2 eq), benzoylperoxide (0.1 eq) at 10° C., stirred at 80° C. 16 hours. Quenched with ice-cold water and extracted with DCM (2×40 mL). Combined organic layer was washed with brine, dried over anhydrous Na₂SO₄ and concentrated to afford 500 mg of 2 as white solid. Similarly, two reactions with 30 g of input starting material gave 12 g and 11 g of 2.

2 (200 mg) in EtOH (6 mL), added NaCN (1.1 eq) at room temperature, reacted at 45° C. 6 hours. Volatiles were removed, residue was diluted with ice-cold water and extracted with EtOAc (2×20 mL), combined organic layer was dried over anhydrous Na₂SO₄, filtered, concentrated, and purified by column [20% EtOAc-hexane] to afford 80 mg of 3 as oily liquid. Similarly, 20 g of 2 gave 10 g of 3 as oily liquid.

Mixed 3 (500 mg), 1,4-dioxane (10 mL), 6-aminohexanol (1.2 eq) at room temperature 20 minutes, added TEA (3.0 eq), mixed at 45° C. 16 hours. Removed volatiles and purified residue on silica flash column to give 520 mg 4 as red thick syrup. Similarly, 10 g of 3 gave 10.5 g of 4.

To 4 (500 mg) in CHCl₃ (20 mL), added PCC (2.5 eq) at 10° C., warmed to room temperature, stirred 4 hours, filtered through CELITE, washed with DCM (20 mL). Filtrate was concentrated and purified on silica to afford 300 mg of 5 as red solid. Similarly, 10 g of 4 gave 6.0 g of 5.

To a stirred solution of 5 (300 mg) in MeOH (15 mL) added DNJ (0.8 eq), AcOH (cat.) at room temperature, stirred 10 minutes, NaCNBH₃ (1.5 eq) added and stirred at room temperature 16 hours. Volatiles were concentrated and material was purified on silica [20% MeOH-EtOAc] to afford 150 mg of 1299. Similarly, with two column purifications, 6.0 g of 5 gave 2.0 g of 1299 as red, low melting solid.

### Example 109: Synthesis of Compound 1300 (N-[3-nitro-4-({6-[(2R,3R,4R,5S)-3,4,5-trihydroxy-2-(hydroxy-methyl)piperidin-1-yl]hexyl}amino)phenyl]methanesulfonamide)

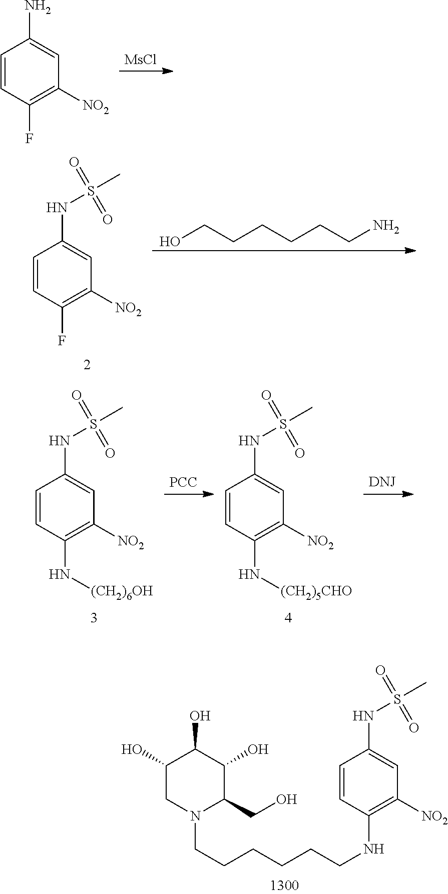

To a stirred solution of 3-nitro-4-fluoroaniline (500 mg) in DCM (10 mL) added N-methylmorpholine (NMM, 3.0 eq) at room temperature, added MsCl (1.2 eq) at 0° C., warmed to room temperature, reacted 2 hours. Quenched with ice-cold water and extracted with DCM (2×40 mL). Combined organic extract was washed with brine, dried over anhydrous Na₂SO₄, concentrated, and purified by silica gel flash column chromatography to afford 400 mg 2. Similarly, 10 g of the aniline gave 8.0 g of 2 as white solid.

2 (500 mg), 1,4-dioxane (10 mL), 6-aminohexanol (1.5 eq), stirred 20 minutes at room temperature, added TEA (3.0 eq), reacted at 55° C. 16 hours. Volatiles were removed and material purified by silica gel flash column chromatography to afford 520 mg 3 as red thick syrup. Similarly, 10 g of 2 yielded 11.0 g of 3 as red solid.

To 3 (500 mg) in CHCl₃ (10 mL) added PCC (2.5 eq) at 10° C., warmed to room temperature, stirred 4 hours. Reaction mass was filtered through CELITE, washed with DCM (20 mL), filtrate was concentrated and purified on silica to afford 200 mg of 4. Similarly, two batches each 5.0 g input 3 gave 2.0 and 2.5 grams of 4 respectively as red solid.

To a stirred solution of 4 (200 mg) in MeOH (5 mL) added DNJ (0.8 eq), AcOH (cat.) at room temperature and stirred 10 minutes, NaCNBH₃ (1.5 eq) was added and stirred at room temperature 16 hours. Volatiles were concentrated and material purified on silica [20% MeOH-EtOAc] to afford 50 mg of 1300. Similarly, two batches with 2-2.5 g input were combined then re-purified by silica chromatography (200-400 mesh) to give 1.3 g of 1300 as orange-red solid.

### Example 110: Synthesis of Compound 1304 (3-nitro-4-({6-[(2R,3R,4R,5S)-3,4,5-trihydroxy-2-(hydroxymethyl)piperidin-1-yl]hexyl}amino)benzonitrile)

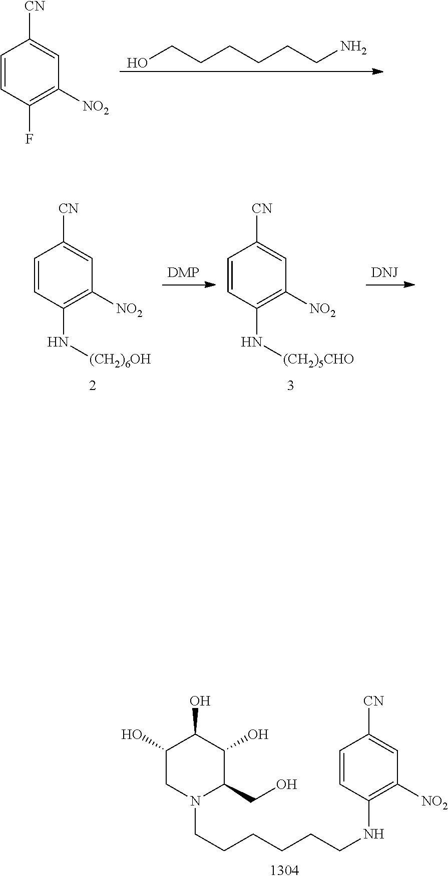

4-Fluoro-3-nitrobenzonitrile (1.0 g), 1,4-dioxane (20 mL), 6-aminohexanol (1.5 eq) stirred at room temperature 20 minutes, added TEA (3.0 eq), reacted at 55° C. 16 hours. Volatiles were removed and the material purified by silica gel flash column chromatography to afford 1.1 g of 2 as yellow solid. Similarly, 8.0 g of the input benzonitrile gave 10.0 g of 2 as yellow solid.

2 (1.0 g), DCM (20 mL), DMP (1.2 eq) added at 0° C., warmed to room temperature, stirred 2 hours. Reaction was quenched with saturated NaHCO₃, extracted with DCM (2×40 mL), organic layer was washed with brine, dried over anhydrous Na₂SO₄ filtered, concentrated, and purified on silica to afford 700 mg of 3. Similarly, 12.0 g of 2 gave 9.0 g of 3 as yellow solid.

3 (700 mg), MeOH (20 mL), DNJ (0.8 eq), AcOH (cat.) stirred at room temperature 10 minutes, added NaCNBH₃ (1.5 eq), stirred at room temperature. Volatiles were concentrated and the material purified on silica [20% MeOH-EtOAc] to afford 350 mg of 1304. 9.0 g of 3 was processed similarly followed by additional trituration with EtOAc (150 mL) to afford 3.5 g of 1304 as yellow solid.

### Example 111: Synthesis of Compound 1305 ((2R,3R,4R,5S)-2-(hydroxymethyl)-1-(3-{4′-methoxy-[1,1′-biphenyl]-4-yl}propyl)piperidine-3,4,5-triol)

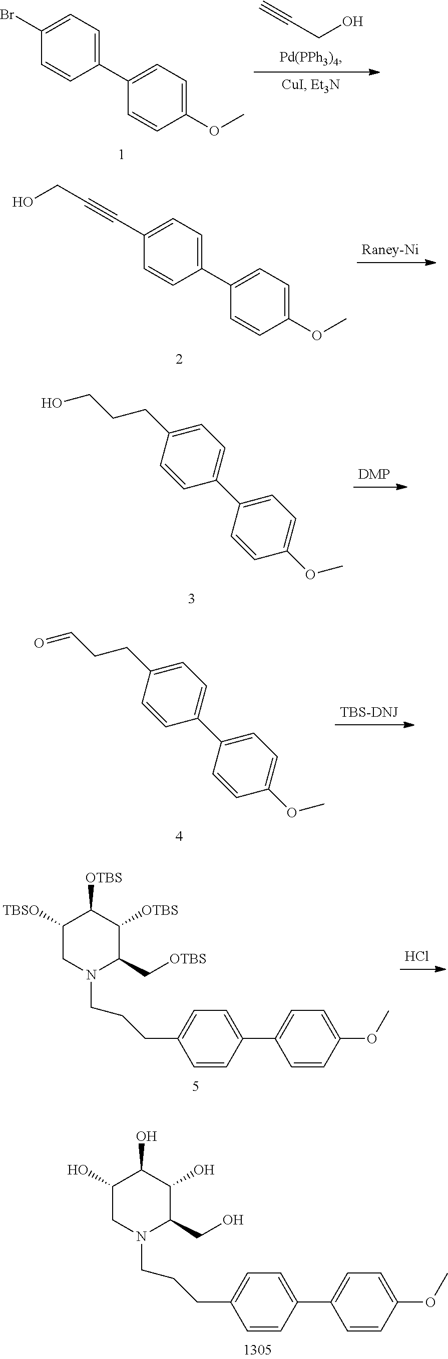

To a stirred solution of 1 (8.0 g) in TEA (150 mL) in a sealed tube added propargyl alcohol (3.4 g), CuI (0.55 g), degassed with Ar 30 minutes, added Pd(PPh₃)₄ (1.76 g) at room temperature, degassed Ar 30 minutes, react at 80° C. 24 hours. Reaction was diluted with ice-cold water and extracted with EtOAc (2×200 mL). Combined organic extracts were washed with brine, dried over anhydrous Na₂SO₄, filtered, concentrated, and purified on silica [20% EtOAc/hexane] to afford 4.7 g of 2 as yellow solid.

To 2 (3.0 g) in THE (60 mL) added Raney-Ni (1.50 g) at room temperature, maintained under H₂ (balloon) 24 hours. Reaction mixture was filtered through a CELITE bed, washed with EtOAc (50 mL), filtrate was concentrated and purified on silica with 50% EtOAc/hexane to afford 2.7 g of 3 as white solid.

3 (3.3 g), DCM (60 mL) added DMP (6.9 g) at 0° C., stirred at room temperature 3 hours. Quenched with saturated NaHCO₃ solution and extracted with DCM (2×50 mL). Combined organic extracts were washed with brine, dried over anhydrous Na₂SO₄ filtered, concentrated, and purified on silica with 15% EtOAc/hexane to afford 2.8 g of 4 as white solid.

To a stirred solution of 4 (3.0 g) in MeOH (60 mL) added TBS-DNJ (7.7 g), AcOH (cat.) at room temperature, stirred 10 minutes, added NaCNBH₃ (1.5 eq), reacted at room temperature for 2 hours. Volatiles were removed and residue purified on silica [2-3% Et₂₀/hexane] to afford 6.5 g of 5 as colorless thick syrup.

Cooled 5 (6.5 g) to 0° C., added 4M HCl in 1,4-dioxane (65 mL), warmed to room temperature and stirred 48 hours. Volatiles were removed, the solid was triturated with DCM (250 mL) and dried under reduced pressure to afford 1.9 g of 1305 as the HCl salt as white solid.

### Example 112: Synthesis of Compound 1306 ((2R,3R,4R,5S)-2-(hydroxymethyl)-1-({4′-methoxy-[1,1′-biphenyl]-4-yl}methyl)piperidine-3,4,5-triol)

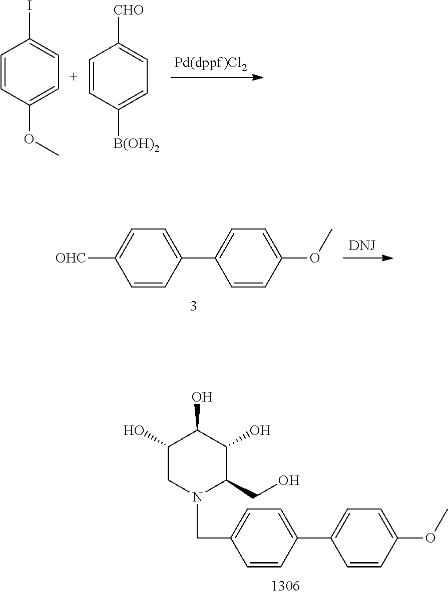

To a stirred mixture of 4-iodoanisole (10 g) in ACN:H₂O (1:1, 300 mL) added (4-formyl-phenyl)boronic acid (6.40 g), degassed with Ar 30 minutes, added Pd(dppf)Cl₂ (0.05 eq), K₂CO₃ (3.0 eq) at room temperature, degassed Ar 30 minutes, warmed to 80° C. 4 hours. Reaction mixture was diluted with ice-cold water and extracted with EtOAc (2×200 mL). Combined organic extracts were washed with brine, dried over anhydrous Na₂SO₄ filtered, concentrated, and purified on silica with 15% EtOAc/hexane to afford 2.5 g of 3 as colorless thick syrup.

3 (5.0 g), MeOH (200 mL), DNJ (3.07 g), AcOH (cat.) stirred for 10 minutes at room temperature, added NaCNBH₃ (2.2 g), stirred at room temperature 48 hours. Volatiles were concentrated and residue purified on silica with 8-10% MeOH in DCM to afford 1.5 g of 1306 as white solid.

### Example 113: Synthesis of Compounds 1308 ((2R,3R,4R,5S)-2-(hydroxymethyl)-1-{3-[4-(2-methoxyethyl)phenyl]propyl}piperidine-3,4,5-triol) and 1307 ((2R,3R,4R,5S)-2-(hydroxymethyl)-1-{5-[4-(2-methoxyethyl)phenyl]pentyl}piperidine-3,4,5-triol)

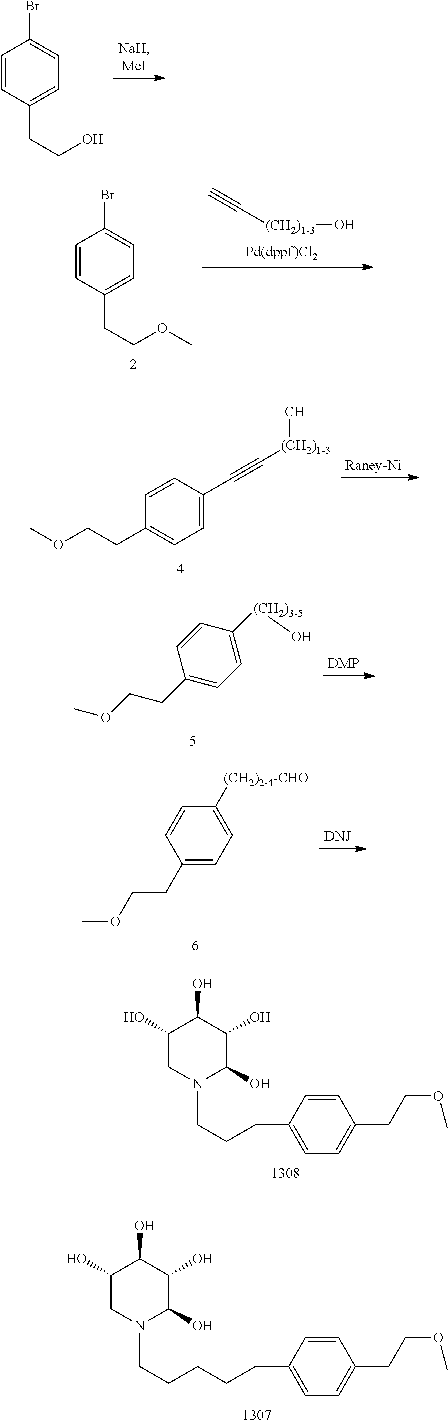

To a stirred solution of 2-(4-bromophenyl)ethan-1-ol (10.0 g) in DMF (100 mL) added NaH (2.29 g) at 0° C., stirred 1 hour at room temperature, added Mel (1.2 eq) at 0° C., warmed to room temperature, stirred 48 hours. Reaction was quenched with ice-cold water (150 mL), extracted with Et₂O (2×100 mL). Combined organic extracts were washed with water (2×100 mL), dried over anhydrous Na₂SO₄, filtered, concentrated, and purified on silica flash column with 10% EtOAc/hexane to afford 6 g of 2 as colorless liquid.

Preparation of 1308 (2R,3R,4R,5S)-2-(hydroxymethyl)-1-{3-[4-(2-methoxyethyl) phenyl]propyl}piperidine-3,4,5-triol: To a stirred solution of 2 (5.0 g) in TEA (100 mL) in a sealed tube, added propargyl alcohol (1.95 g) and CuI (0.43 g), purged with Ar for 30 minutes, Pd(dppf)Cl₂ (1.68 g) added at room temperature, degassed Ar for 30 minutes, maintained at 80° C. 16 hours. Cooled to room temperature, diluted with ice-cold water, extracted with EtOAc (2×100 mL). Combined organic extracts were washed with brine, dried over anhydrous Na₂SO₄, filtered, concentrated, and purified on silica flash column with 30% EtOAc/hexane to afford 2 g of 4 as colorless thick syrup.

To a stirred solution of 4 (4.2 g) in THE (80 mL) added Raney-Ni (4.0 g) in a Parr shaker at room temperature, stirred under H₂ (60 psi) 16 hours. Mix was filtered through a CELITE bed and filtrate was concentrated then purified on silica flash column with 20% EtOAc/hexane to afford 2.9 g of 5 as colorless thick syrup.

To a stirred solution of 5 (2.2 g) in DCM (50 mL) added DMP (5.76 g) at 0° C., stirred at room temperature 3 hours. Reaction was quenched with saturated NaHCO₃ solution, extracted with DCM (2×40 mL), combined organic extracts were washed with brine, dried over anhydrous Na₂SO₄, filtered, concentrated, and purified on silica flash column with 20% EtOAc/hexane to afford 1.8 g of 6 as colorless thick syrup.

To a stirred solution of 6 (1.6 g) in MeOH (50 mL) added DNJ (1.08 g), AcOH (cat.) at room temperature, stirred 10 minutes, added Pd/C (1.5 g) and stirred at room temperature under H₂ (balloon) for 16 hours. Filtered through CELITE bed, washed with MeOH (50 mL), concentrated filtrate and purified on silica with 8-10% MeOH/DCM to afford 1.7 g of 1308 as low melting solid.

Preparation of 1307 (2R,3R,4R,5S)-2-(hydroxymethyl)-1-{5-[4-(2-methoxyethyl) phenyl]pentyl}piperidine-3,4,5-triol: To a stirred solution of 2 (5.1 g) in TEA (100 mL) in a sealed tube, added pent-4-yn-1-ol (1.59 g), CuI (0.45 g), purged with Ar 30 minutes, added Pd(dppf)Cl₂ (1.73 g) at room temperature, degassed Ar 30 minutes, heated to 80° C. and maintained 16 hours. Diluted with ice-cold water, extracted with EtOAc (2×100 mL), combined organic extracts were washed with brine, dried over anhydrous Na₂SO₄ filtered, concentrated, and purified on silica with 20% EtOAc/hexane to afford 5.5 g of 4 as colorless thick syrup.

To a stirred solution of 4 (5.5 g) in DCM (100 mL) added DMP (12.8 g) at 0° C., stirred at room temperature 3 hours. Mixture was quenched with saturated NaHCO₃ solution, extracted with DCM (2×100 mL), combined organic extracts were washed with brine, dried over anhydrous Na₂SO₄ filtered, concentrated, and purified on silica with 15% EtOAc/hexane to afford 4.2 g of 5 as colorless thick syrup.

To a stirred solution of 5 (4.2 g) in MeOH (100 mL) added DNJ (2.54 g), AcOH (cat.) at room temperature, stirred 10 minutes, added Pd/C (2.0 g), stirred at room temperature under H₂ (balloon) 16 hours. Filtered through CELITE bed, washed with MeOH (100 mL), filtrate was concentrated and purified on silica with 8-10% MeOH/DCM to afford 1.8 g of 1307 as low melting solid.

### Example 114: Synthesis of Compound 1311 ((2R,3R,4R,5S)-2-(hydroxymethyl)-1-[8-(morpholin-2-yl)octyl]piperidine-3,4,5-triol)

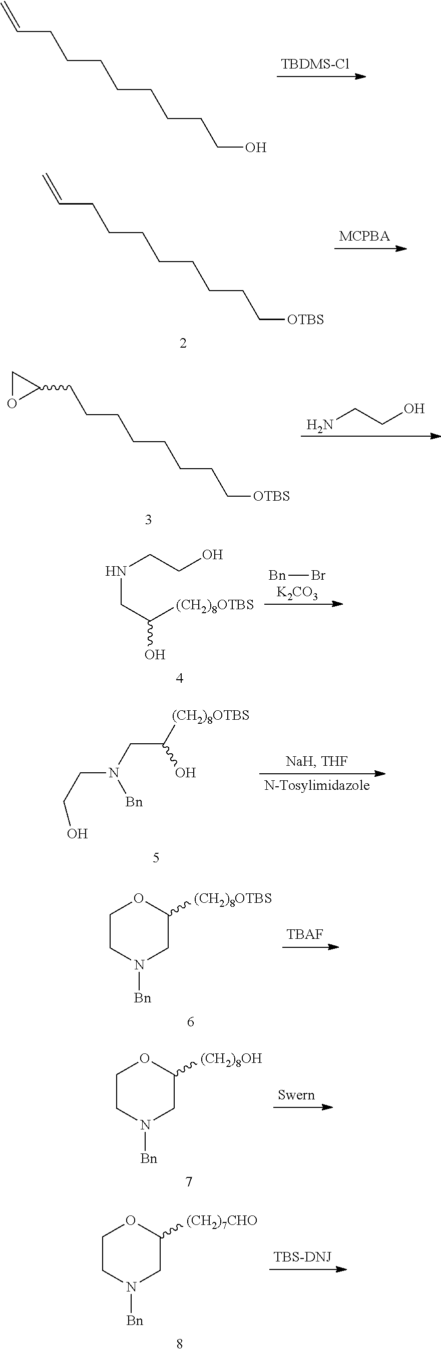

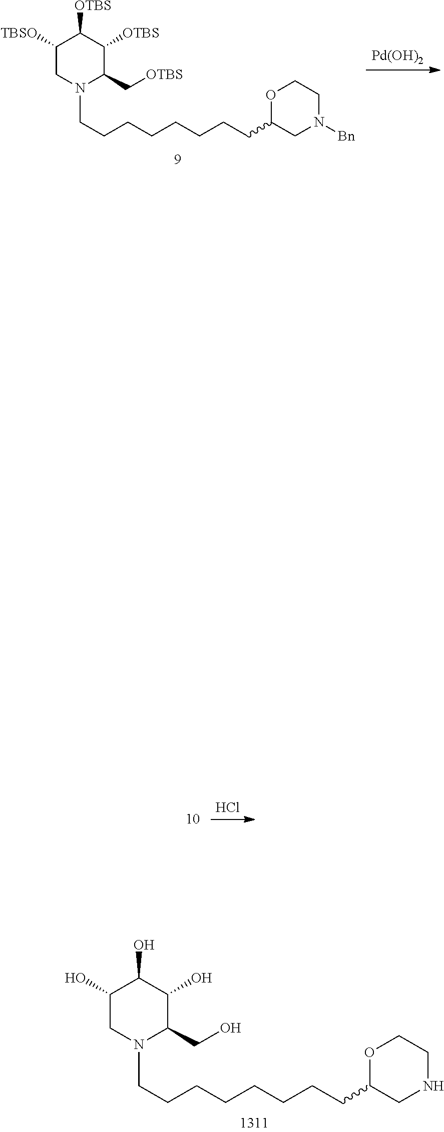 [SMILES: CC(C)(C)[Si](C)(C)OC[C@@H]1[C@@H](O[Si](C)(C)C(C)(C)C)[C@H](O[Si](C)(C)C(C)(C)C)[C@@H](O[Si](C)(C)C(C)(C)C)CN1CCCCCCCCC1CN(Cc2ccccc2)CCO1 ; "Pd(OH)2" ; Cl ; OC[C@@H]1[C@@H](O)[C@H](O)[C@@H](O)CN1CCCCCCCCC1CNCCO1]

To a stirred solution of 9-decen-1-ol (1 g) in DMF (8 mL) added imidazole (1.3 eq) at room temperature, after 30 minutes added TBDMS-Cl (1.2 eq) at 0° C., warmed to room temperature and maintained 2 hours. The reaction mass was diluted with ice-cold water and extracted with EtOAc (2×100 mL). Combined organic extracts were washed with brine, dried over anhydrous Na₂SO₄, concentrated, and purified on silica to afford 1.5 g of 2 as colorless thick syrup.

To 2 (1.0 g) in DCM (20 mL) added MCPBA (2.5 eq) at 0° C., stirred at room temperature 16 hours. Quenched with saturated NaHCO₃ solution and extracted with DCM (2×20 mL). Combined organic extracts were washed with saturated NaHSO₃ then brine, dried over anhydrous Na₂SO₄, concentrated, and purified by silica flash column to afford 850 mg of 3 as thick syrup.

To 3 (1 g) in EtOH (30 mL) added ethanolamine (10 mL) at 0° C., reacted at 55° C. for 6 hours. Volatiles were removed and residue diluted with ice-cold water and extracted with EtOAc (2×20 mL). The combined organic extracts were washed with brine, dried over anhydrous Na₂SO₄ and concentrated to afford 1.1 g of 4 as thick syrup.

To 4 (600 mg) in DMF (20 mL) added K₂CO₃ (2.0 eq) at room temperature, stirred 30 minutes, added benzyl bromide (1.0 eq) at 0° C., stirred at room temperature 16 hours. Reaction mass was diluted with ice-cold water and extracted with EtOAc (2×50 mL). The combined organic extracts were washed with brine, dried over anhydrous Na₂SO₄, concentrated, and purified by silica gel flash column to afford 500 mg of 5 as thick syrup.

To a stirred solution of imidazole (4.12 g) in DCM (100 mL) added tosyl chloride (1.0 eq) at 0° C., stirred 4 hours at room temperature. Reaction mass was diluted with ice-cold water and extracted with EtOAc (2×50 mL). The combined organic extracts were washed with brine, dried over anhydrous Na₂SO₄ and concentrated under reduced pressure to afford 6.2 g of N-tosyl imidazole as white solid.

To 5 (500 mg) in THE (10 mL) added NaH (2.5 eq) at 0° C., stirred for 1 hour at room temperature then added N-tosylimidazole (1.2 eq) at 0° C., warmed to room temperature and reacted 16 hours. Reaction mass was diluted with ice-cold water and extracted with EtOAc (2×20 mL). The combined organic extracts were washed with brine, dried over anhydrous Na₂SO₄, concentrated, and purified on silica to afford 250 mg of 6 as thick syrup.

To 6 (350 mg) in THE (5 mL) added 1 M TBAF in THE (3.0 eq) at 0° C., stirred at room temperature 4 hours. Quenched with ice-cold water and extracted with EtOAc (2×25 mL). Combined organic extracts were washed with brine solution, dried over anhydrous Na₂SO₄, concentrated, and purified on silica to afford 220 mg of 7 as thick syrup.

To (COCl)₂ (2.1 eq) in dry THF (5 mL) added DMSO (2.6 eq) at −78° C., stirred 30 minutes, added 7 (459 mg in 3 mL dry THF) and continued for 2 hours at −78° C., added TEA (5.4 eq) and maintained for 2 hours. Quenched with ice-cold water and extracted with DCM (2×20 mL). Combined organic extracts were washed with brine solution, dried over anhydrous Na₂SO₄, concentrated, and purified on silica to afford 390 mg of 8 as thick syrup.

To 8 (150 mg) in MeOH (10 mL) added TBS-DBJ (0.8 eq), AcOH (cat.) at room temperature and stirred for 10 minutes. Added NaCNBH₃ (1.5 eq) and stirred at room temperature 16 hours. Volatiles were concentrated and residue purified on silica to afford 200 mg of 9 as thick syrup.

To 9 (200 mg) in MeOH (5 mL) added Pd(OH)₂ (400 mg) and stirred at room temperature under H₂ balloon pressure for 16 hours. Reaction mass was filtered through CELITE bed and washed with excess MeOH. Filtrate was concentrated and the residue purified on silica to afford 150 mg of 10 as thick syrup.

10 (150 mg), 1,4-dioxane (2 mL) added 4M HCl in 1,4 dioxane (2 mL) at 0° C., stirred at room temperature for 16 hours. Mass directed purification gave 30 mg of 1311 as colorless thick syrup.

### Example 115: Synthesis of Compounds 1316 ((2R,3R,4R,5S)-2-(hydroxymethyl)-1-{[4-({[3-methyl-5-(2H-1,2,3-triazol-2-yl)phenyl]amino}methyl)phenyl]methyl}piperidine-3,4,5-triol) and 1323 ((2R,3R,4R,5S)-2-(hydroxymethyl)-1-{[4-({[3-methyl-5-(1H-1,2,3-triazol-1-yl)phenyl]amino}methyl)phenyl]methyl}piperidine-3,4,5-triol)

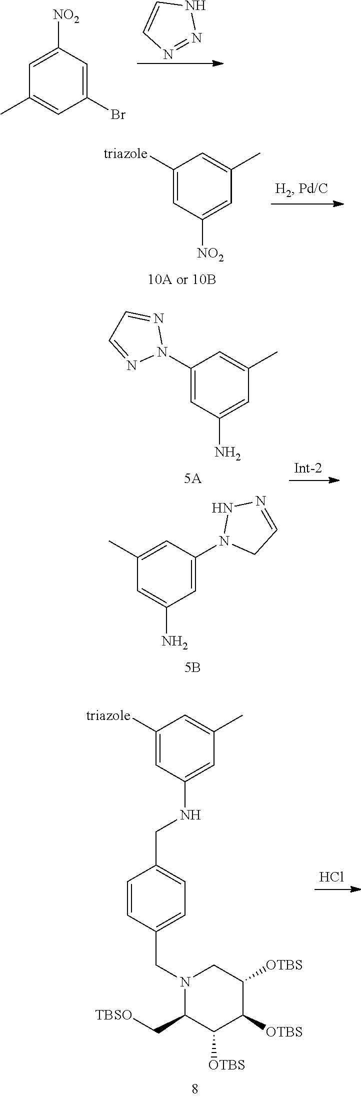

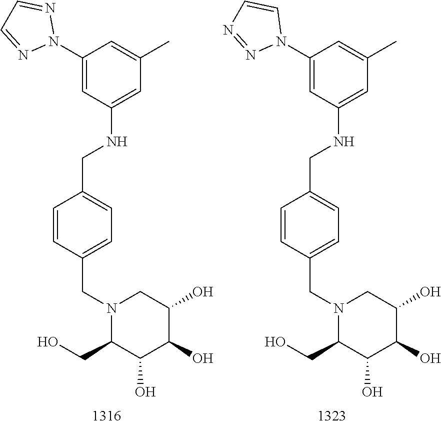 [SMILES: Cc1cc(NCc2ccc(CN3C[C@H](O)[C@@H](O)[C@H](O)[C@H]3CO)cc2)cc(-n2nccn2)c1 ; Cc1cc(NCc2ccc(CN3C[C@H](O)[C@@H](O)[C@H](O)[C@H]3CO)cc2)cc(-n2ccnn2)c1]

Mixed 3-bromo-5-nitrotoluene (2.0 g), 1,2,3-triazole (15.0 eq), K₂CO₃ (3 eq), Cu powder (3.0 eq) at room temperature then at 120° C. for 16 hours cooled to room temperature, diluted with EtOAc (500 mL) and filtered through a bed of CELITE. The filtrate was concentrated and residue purified by COMBIFLASH [1% EtOAc in hexane] to afford 350 mg of 10A (2-triazole isomer) and 300 mg of 10B (1-triazole isomer).

10 (350 mg A or 300 mg B), EtOAc (10 mL), 10% Pd/C (50% wet, 122 mg for A, 100 mg for B), reacted under H₂ (balloon pressure) at room temperature for 6 hours. Each reaction was filtered through a bed of CELITE and washed with excess of EtOAc. Each filtrate was concentrated and purified by COMBIFLASH [both 10% EtOAc in hexane] to afford respectively 250 mg 5A and 140 mg 5B.

Preparation of 1316 (2R,3R,4R,5S)-2-(hydroxymethyl)-1-{[4-({[3-methyl-5-(2H-1,2,3-triazol-2-yl)phenyl]amino}methyl)phenyl]methyl}piperidine-3,4,5-triol: Mixed Int-2 (2.0 g, Example 2), MeOH:DCM (40 mL. 1:1), 5A (1.0 eq), AcOH (cat.) 10 minutes at room temperature, added NaCNBH₃ (1.5 eq) at 0° C., mixed 16 hours. Removed volatiles, diluted residue with water (30 mL), extracted with EtOAc (2×30 mL). The organic layer was washed with water, dried over anhydrous Na₂SO₄, filtered, concentrated, and purified by COMBIFLASH [2% EtOAc/hexane] to afford 900 mg of 8.

Mixed 8 (900 mg), DCM (20 mL) at 0° C., added 4.0 M HCl in 1,4-dioxane (9 mL), mixed at room temperature for 16 hours. Reaction was concentrated and the residue was triturated with DCM to provide 250 mg of 1316 as HCl salt.

Preparation of 1323 (2R,3R,4R,5S)-2-(hydroxymethyl)-1-{[4-({[3-methyl-5-(1H-1,2,3-triazol-1-yl)phenyl]amino}methyl)phenyl]methyl}piperidine-3,4,5-triol: Mixed Int-2 (2.0 g), MeOH:DCM (40 mL. 1:1), 5B (1.0 eq), AcOH (cat.) 10 minutes at room temperature, added NaCNBH₃ (1.5 eq) at 0° C., stirred 16 hours. Removed volatiles, diluted the residue with water (20 mL) and extracted with EtOAc (2×20 mL). The organic layer was washed with water, dried over anhydrous Na₂SO₄, filtered, concentrated, and purified by COMBIFLASH [18% EtOAc/hexane] to afford 240 mg of 8.

Mixed 8 (240 mg) in DCM (10 mL) at 0° C., added 4.0 M HCl in 1,4-dioxane (2.5 mL) then stirred at room temperature 16 hours. Concentrated the reaction and triturated the residue with DCM to obtain 80 mg of 1323 as HCl salt.

### Example 116: Synthesis of Compound 1324 ((2R,3R,4R,5S)-1-{[4-({[4-(furan-2-yl)-2-methylphenyl]amino}methyl)phenyl]methyl}-2-(hydroxymethyl)piperidine-3,4,5-triol)

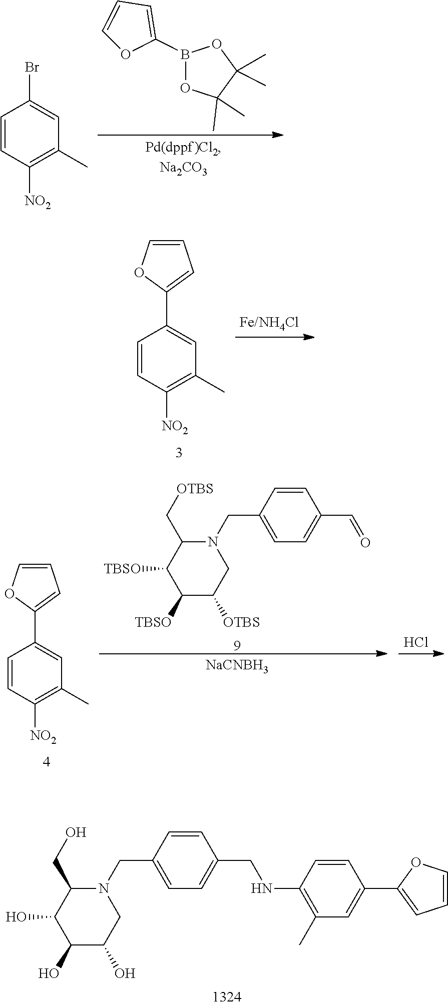

Degassed 2-nitro-4-bromotoluene (50 g), EtOH:toluene:water (1:1:0.5, 750 mL) for 30 minutes, added 2-furanyl boronate (1.5 eq), Pd(dppf)Cl₂ (0.1 eq), Na₂CO₃ (3 eq), heated at 70° C. for 1 hour. The reaction was filtered through CELITE bed, washed with EtOAc (1 L), filtrate was concentrated, residue was dissolved in EtOAc (2 L) and washed with water. The organic layer was dried over Na₂SO₄, filtered, concentrated, and purified by COMBIFLASH [0.5% EtOAc in hexane] to afford 35 g of 3.

Reacted 3 (35 g), EtOH:water (1:1, 1 L), Fe (6 eq), NH₄Cl (6 eq) at 80° C. 16 hours. Reaction was filtered through CELITE bed, washed with EtOAc (2 L), filtrate was concentrated, residue was dissolved in EtOAc (3 L) and washed with water. The organic layer was dried over Na₂SO₄, filtered, concentrated, and purified by COMBIFLASH [30% EtOAc-hexane] to afford 26 g of 4.

9 (60 g, as prepared above), DCM (60 mL), MeOH (600 mL), 4 (19.95 g), AcOH (cat.) stirred 30 minutes at room temperature, NaCNBH₃ (2.0 eq) added at 0° C. then reacted at room temperature 16 hours. Volatiles are removed, residue was diluted with water (700 mL) and extracted with EtOAc (2×1 L), organic layer was washed with water, dried over anhydrous Na₂SO₄, filtered, concentrated, and purified by COMBIFLASH [7% EtOAc-hexane] to afford 45 g of 10.

Mixed 10 (22.5 g), MeOH:DCM (1:1, 400 mL), 4.0 M HCl in 1,4-dioxane (200 mL) at 0° C. then at room temperature for 6 hours. Volatiles were removed, residue was washed with Et₂O (2×300 mL) then diluted with water (200 mL) and washed with EtOAc (2×200 mL). The aqueous layer was neutralized with NaHCO₃ and extracted with 5% MeOH in EtOAc (3×400 mL). The organic layer was dried over anhydrous Na₂SO₄, concentrated, slurried in EtOAc (200 mL) and stirred 30 minutes at room temperature. The solids were filtered, washed with pentane and dried under vacuum. Combined batches (22 g) were slurried with n-pentane (500 mL), filtered, and dried to provide 21.7 g of 1324.

### Example 117. Synthesis of Compounds 1319 ((2R,3R,4R,5S)-2-(hydroxymethyl)-1-{[5-({[3-methyl-5-(pyrimidin-2-yl)phenyl]amino}methyl)pyrazin-2-yl]methyl}piperidine-3,4,5-triol), 1327 ((2R,3R,4R,5S)-1-{[5-({[3-(furan-2-yl)-5-methylphenyl]amino}methyl)pyrazin-2-yl]methyl}-2-(hydroxymethyl)piperidine-3,4,5-triol), 1329 ((2R,3R,4R,5S)-1-{[5-({[3-cyclopropyl-5-(pyrimidin-2-yl)phenyl]amino}methyl)pyrazin-2-yl]methyl}-2-(hydroxymethyl)piperidine-3,4,5-triol), 1330 ((2R,3R,4R,5S)-2-(hydroxymethyl)-1-{[5-({[3-methyl-5-(1H-pyrrol-2-yl)phenyl]amino}methyl)pyrazin-2-yl]methyl}piperidine-3,4,5-triol), and 1332 ((2R,3R,4R,5S)-2-(hydroxymethyl)-1-{[5-({[3-methyl-5-(morpholin-4-yl)phenyl]amino}methyl)pyrazin-2-yl]methyl}piperidine-3,4,5-triol)

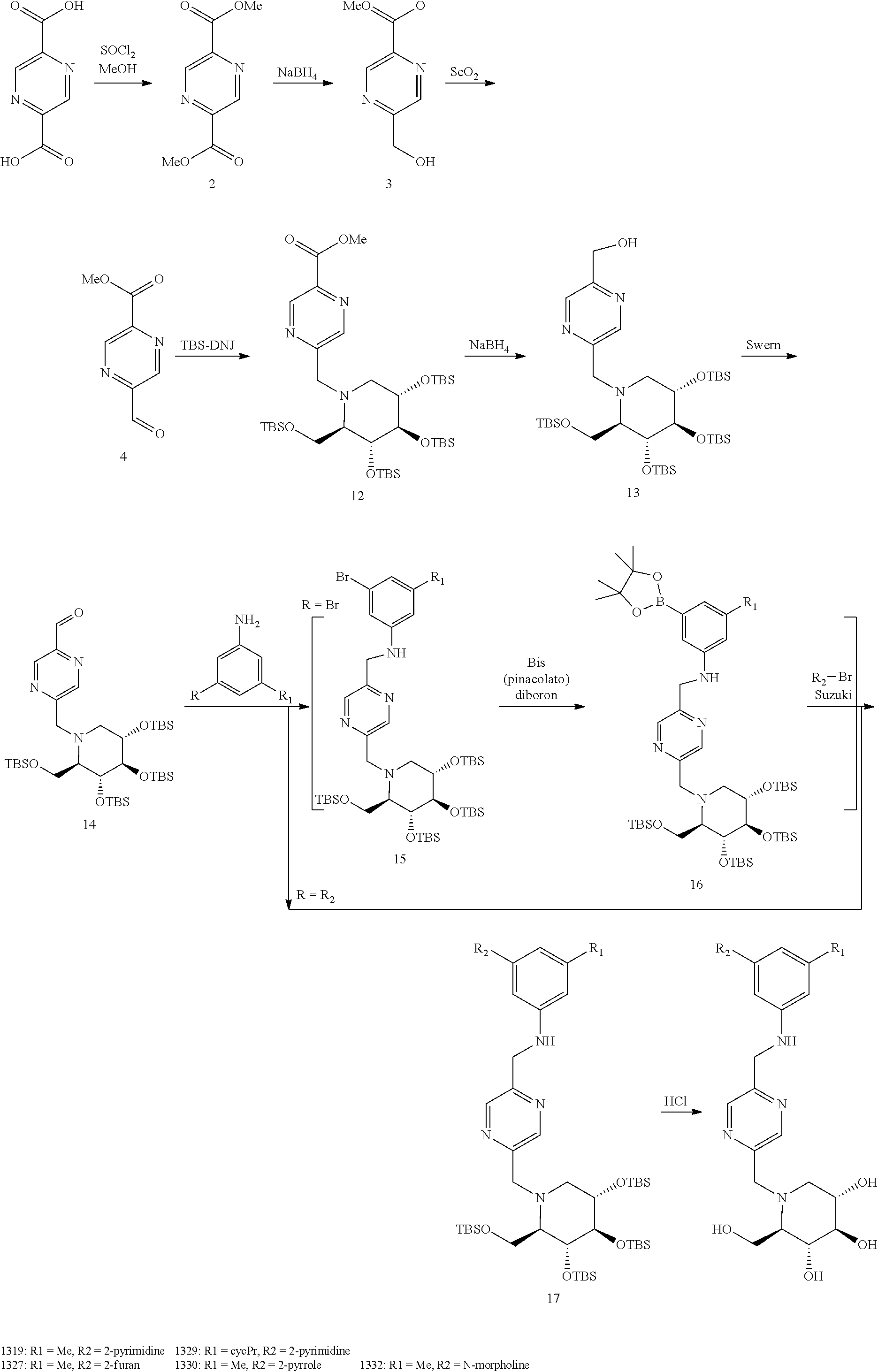

Pyrazine-2,5-dicarboxylic acid (5 g), MeOH (125 mL), SOCl₂ (15 mL) reacted 80° C. for 48 hours. The mixture was concentrated and residue was dissolved in EtOAc (100 mL) and washed with water. Organic layer was dried over Na₂SO₄, filtered and concentrated to afford 4.8 g of diester 2.

2 (4 g), MeOH/DCM (50 mL, 7:3) mixed at 0° C., added NaBH₄ (1.5 eq) and reacted 1.5 hours added another 1.5 eq. NaBH₄ (1.5 eq) stirred at 0° C. additional 30 minutes. Reaction was quenched with saturated aqueous NH₄Cl and extracted with DCM (2×100 mL). Organic layer was dried over Na₂SO₄, filtered, concentrated, and purified on silica [50% EtOAc in hexane] to afford 2 g of 3.

3 (2 g), SeO₂ (0.6 eq), 1,4-dioxane (100 mL) reacted 4 hours at 65° C. Reaction was filtered through a bed of CELITE, filtrated and evaporated to afford 2 g of aldehyde 4.

Mixed 4 (2 g), MeOH (40 mL), TBS-DNJ (0.6 eq), AcOH (cat.) 10 minutes at room temperature, added NaCNBH₃ (1.5 eq) at 0° C. then room temperature for 16 hours. Reaction was concentrated, residue dissolved in EtOAc (50 mL) and washed with water. Organic layer was dried over Na₂SO₄, filtered, concentrated, and purified by column [50% EtOAc in hexane] to afford 3 g of 12.

Mixed 12 (3 g), MeOH/DCM (2:1, 25 mL) at 0° C., added NaBH₄ (20 eq), reacted 24 hours at room temperature. The mixture was concentrated and residue was dissolved in EtOAc (50 mL) and washed with water. Organic layer was dried over Na₂SO₄, filtered, concentrated, and purified by column [20% EtOAc in hexane] to afford 1.4 g of 13.

(COCl)₂ (3.0 eq) in DCM (7.0 mL) and DMSO (4.0 eq) mixed at −78° C. 30 minutes, added 13 (150 mg) in DCM (7.0 mL) at −78° C. for 1 hour, added Et₃N (5 eq) at −78° C., increased to room temperature and stirred 2 hours. The reaction was diluted with water (15.0 mL), extracted with DCM (2×15 mL), the organic layer was washed with water, dried over anhydrous Na₂SO₄, filtered, concentrated, and purified by COMBIFLASH [3% EtOAc/Hexane] to obtain 80 mg of 14 as colorless syrup. This aldehyde was prepared as required and used immediately.

Preparation of 1329 (2R,3R,4R,5S)-1-{[5-({[3-cyclopropyl-5-(pyrimidin-2-yl) phenyl]amino}methyl)pyrazin-2-yl]methyl}-2-(hydroxymethyl)piperidine-3,4,5-triol: 14 (70 mg), DCM (0.5 mL), MeOH (4 mL), 3-cyclopropyl-5(2-pyrimidinyl)aniline (1 eq, Example 10), AcOH (cat.) mixed 10 minutes at room temperature, NaCNBH₃ (1.5 eq) added at 0° C. then stirred at room temperature 16 hours. Volatiles are removed, residue was diluted with water (5 mL) and extracted with EtOAc (2×10 mL), the organic layer was washed with water and dried over anhydrous Na₂SO₄, filtered, concentrated, and purified by COMBIFLASH [15% EtOAc-hexane] to obtain 40 mg of 17 as colorless syrup.

Mixed 17 (40 mg), MeOH:DCM (1:1, 4 mL), 4.0 M HCl in 1, 4-dioxane (2.0 mL), at 0° C. then at room temperature 6 hours. Removed volatiles and residue was washed with Et₂O (2×5 mL) then diluted with water (2 mL) and washed with EtOAc (2×4 mL). The aqueous layer was basified with NaHCO₃ and extracted with 5% MeOH in EtOAc (3×6 mL). The organic layer was dried over anhydrous Na₂SO₄ and concentrated. Two batches were combined and lyophilized to obtain 20 mg of 1329 as off-white solid.

Preparation of 1330 (2R,3R,4R,5S)-2-(hydroxymethyl)-1-{[5-({[3-methyl-5-(1H-pyrrol-2-yl)phenyl]amino}methyl)pyrazin-2-yl]methyl}piperidine-3,4,5-triol: Mixed 14 (170 mg), DCM (2.5 mL), MeOH (5.0 mL), 3-methyl-5(2-pyrrolyl)aniline (1.0 eq), AcOH (cat.) 10 minutes at room temperature, added NaCNBH₃ (1.5 eq) at 0° C., reacted at room temperature 16 hours. Removed volatiles, diluted residue with water (10 mL) and extracted with EtOAc (2×20 mL). The organic layer was washed with water, dried over anhydrous Na₂SO₄, filtered, concentrated, and purified by COMBIFLASH [20% EtOAc/Hexane] to give 80 mg of 3.

Mixed 3 (100 mg), DCM (2.0 mL), MeOH (2.0 mL) at 0° C., added 4.0 M HCl in 1,4-dioxane (1 mL), stirred at room temperature for 8 hours. Volatiles was concentrated, added water (5 mL) to residue and washed with EtOAc (3×3 mL). The aqueous layer was adjusted to pH=8 with saturated NaHCO₃ solution and extracted with EtOAc (3×5 mL). Basic organic extracts were concentrated, triturated with Et₂O, and lyophilized to obtain 16 mg of 1330.

Preparation of 1332 (2R,3R,4R,5S)-2-(hydroxymethyl)-1-{[5-({[3-methyl-5-(morpholin-4-yl)phenyl]amino}methyl)pyrazin-2-yl]methyl}piperidine-3,4,5-triol: 14 (120 mg), DCM (0.5 mL), MeOH (5.0 mL), 3-methyl-5-(N-morpholinyl)aniline (1.0 eq), AcOH (cat.) mixed 10 minutes at room temperature, added NaCNBH₃ (1.5 eq) at 0° C. then reacted 16 hours at room temperature. Volatiles were removed, the residue was diluted with water (5 mL) and extracted with EtOAc (2×10 mL). The organic layer was washed with water, dried over anhydrous Na₂SO₄, filtered, concentrated, and purified by COMBIFLASH [15% EtOAc/hexane] to obtain 80 mg of 3 as light yellow syrup.

Mixed 3 (80 mg), MeOH:DCM (1:1, 5 mL), 4.0 M HCl in 1,4-dioxane (2.5 mL) at 0° C. then 6 hours at room temperature. Removed volatiles, washed residue with Et₂₀ (2×5 mL) then diluted with water (2 mL) and washed with EtOAc (2×4 mL). The aqueous layer was basified with NaHCO₃ and extracted with 5% MeOH in EtOAc (3×6 mL). Combined organic layers were dried over anhydrous Na₂SO₄ and concentrated to afford 25 mg of 1332 as off white solid.

Preparation of 1319 (2R,3R,4R,5S)-2-(hydroxymethyl)-1-{[5-({[3-methyl-5-(pyrimidin-2-yl)phenyl]amino}methyl)pyrazin-2-yl]methyl}piperidine-3,4,5-triol: Mixed 14 (150 mg), DCM (2 mL), MeOH (10 mL), 3-methyl-5-(pyrimidin-2-yl)aniline (1.0 eq), AcOH (cat.) 10 minutes at room temperature, added NaCNBH₃ (1.5 eq) at 0° C., mixed at room temperature 16 hours. Removed volatiles, diluted residue with water (10 mL), extracted with EtOAc (2×20 mL). The organic layer was washed with water, dried over anhydrous Na₂SO₄, filtered, concentrated, and purified by COMBIFLASH [20% EtOAc/Hexane] to give 80 mg of 17 as light yellow syrup.

Mixed 17 (80 mg), MeOH:DCM (1:1, 5 mL), 4.0 M HCl in 1,4-dioxane (2.5 mL) at 0° C. then at room temperature for 6 hours. Volatiles were removed and residue washed with Et₂O (2×5 mL) then diluted with water (5 mL) and washed with EtOAc (2×15 mL). The aqueous layer was basified with NaHCO₃ and extracted with 5% MeOH in EtOAc (5×15 mL). Combined organic layer was dried over anhydrous Na₂SO₄ and concentrated to give 25 mg of 1319 as off white solid.

Preparation of 1327 (2R,3R,4R,5S)-1-{[5-({[3-(furan-2-yl)-5-methylphenyl]amino}methyl)pyrazin-2-yl]methyl}-2-(hydroxymethyl)piperidine-3,4,5-triol: Mixed 14 (150 mg), DCM (2 mL), MeOH (10 mL), 3-(furan-2-yl)-5-methylaniline (1.0 eq), AcOH (cat.) 10 minutes at room temperature, added NaCNBH₃ (1.5 eq), mixed at 0° C. then 16 hours at room temperature. Removed volatiles, diluted residue with water (10 mL) and extracted with EtOAc (2×20 mL). The organic layer was washed with water and dried over anhydrous Na₂SO₄, filtered, concentrated, and purified by COMBIFLASH [10% EtOAc/hexane] to obtain 100 mg of 3 as light yellow syrup.

Mixed 3 (100 mg), MeOH:DCM (1:1, 6 mL), 4.0 M HCl in 1,4-dioxane (3 mL) at 0° C. then at room temperature for 6 hours. Volatiles were removed and the residue washed with Et₂O (2×5 mL). The residue was diluted with water (5 mL) and washed with EtOAc (2×15 mL). The aqueous layer was basified with NaHCO₃ solution and extracted with 5% MeOH in EtOAc (3×15 mL). The organic layer was dried over anhydrous Na₂SO₄ then concentrated under reduced pressure to afford 40 mg of 1327 as off white solid.

### Preparation of Anilines for Examples 117 and 118

3-(Furan-2-yl)-5-methylaniline: To 1-bromo-3-methyl-5-nitrobenzene ((4 g), toluene:EtOH:water (1:1:0.5, 100 mL) and 2-(furan-2-yl)boronate (1.5 eq) added Na₂CO₃ (3 eq), degassed for 30 minutes, then added Pd(dppf)Cl₂ (0.1 eq), reacted 70° C. for 1 hour. The reaction mass was concentrated and the residue dissolved in EtOAc (150 mL) and water, filtered through CELITE bed then washed with EtOAc (50 mL). The organic layer was dried over Na₂SO₄, filtered, concentrated, and purified by COMBIFLASH [5% EtOAc in hexane] to afford 2.5 g of the furyl-nitrotoluene. 1 g of this material was mixed in EtOH:H₂O (4:1, 20 V), then added Fe (5.0 eq), NH₄Cl (5.0 eq) then reacted at 80° C. for 2 hours. Reaction mass was filtered on CELITE bed, washed the bed with EtOAc (3×10 mL). Combined filtrates were washed with water and the organic layer was separated and dried over Na₂SO₄, filtered, concentrated, and purified by COMBIFLASH [15% EtOAc in hexane] to afford 800 mg of 3-(furan-2-yl)-5-methylaniline (used for 1334 and 1327).

3-Methyl-5-morpholinoaniline: To 1-bromo-3-methyl-5-nitrobenzene (4 g) in toluene (10 mL) and morpholine (5.0 eq) added tBuONa (1.5 eq), degassed for 30 minutes, then added BINAP (0.2 eq), Pd₂(dba)₃ (0.1 eq) and reacted at 80° C. for 6 hours. The reaction mass was concentrated and the residue was dissolved in EtOAc (80 mL) and water (30 mL) and filtered through CELITE bed and washed with EtOAc (50 mL). The organic layer was separated and dried over Na₂SO₄, filtered, concentrated, and purified by COMBIFLASH [5% EtOAc in Hexane] to afford 2.7 g of the morpholino-nitrotoluene. 1 g of this material was reduced in EtOH/water (1:1, 20 mL) with Fe (5.0 eq) and NH₄Cl (5.0 eq) at 80° C. for 1 hour. The reaction was concentrated and the residue was dissolved in EtOAc, filtered through CELITE bed, and washed with EtOAc (10 mL). The filtrate was washed with water and the organic layer was dried over Na₂SO₄, filtered, concentrated, and purified by COMBIFLASH [eluting with 30% EtOAc:hexane] to afford 700 mg of 3-methyl-5-morpholinoaniline (used for 1332 and 1336).

3-Methyl-5-(oxazol-2-yl)aniline: Purged 1-bromo-3-methyl-5-nitrobenzene (500 mg) in 1,4-dioxane (10 mL) for 30 minutes under N₂, added bispinacalato diboron (1.5 eq), KOAc (3.0 eq), Pd(dppf)Cl₂ (0.1 eq), reacted at 100° C. for 6 hours. Mixture was concentrated and the residue dissolved in water (10 mL) and extracted with EtOAc (3×10 mL). The organic layer was separated and concentrated to afford the nitrotoluene-boronate. This boronate (300 mg) was mixed with 2-bromooxazole (2.0 eq) in EtOH:toluene:water (1:1:1, 6 mL) and Na₂CO₃ (3.0 eq), degassed for 30 minutes, then added Pd(dppf)Cl₂ (0.1 eq) and reacted at 70° C. for 16 hours. The reaction mixture was distilled and the residue was dissolved in water and extracted with EtOAc (2×15 mL). The organic layer was dried over Na₂SO₄, filtered and concentrated under reduced pressure to afford 90 mg of the oxazolyl-nitrotoluene. 60 mg of this material was reduced in EtOH/water (1:1, 12 mL) by reacting with Fe (3.0 eq), NH₄Cl (3.0 eq) at 80° C. for 2 hours. Reaction mixture was distilled and the residue dissolved in EtOAc (30 mL), filtered through CELITE bed, the bed washed with EtOAc (2×15 mL). The combined filtrates were washed with water (10 mL), dried over anhydrous Na₂SO₄, filtered, concentrated, and purified by COMBIFLASH [70% EtOAc in hexane] to afford 35 mg of 3-methyl-5-(oxazol-2-yl)aniline (used for 1337).

3-methyl-5-(1H-pyrrol-2-yl)aniline: To 1-bromo-3-methyl-5-nitrobenzene (500 mg) in EtOH:toluene:water (1:1:0.5, 6 mL) with 1-BOC-pyrrole-2-boronate (1.5 eq), added Na₂CO₃ (3.0 eq), degassed under N₂ 30 minutes, then added Pd(dppf)Cl₂ (0.1 eq), reacted at 80° C. for 2 hours. Reaction mass was concentrated and the residue was dissolved in EtOAc (30 mL) and water and filtered through CELITE bed and washed with EtOAc (50 mL). The organic layer was separated and dried over Na₂SO₄, filtered and concentrated. The material was purified by COMBIFLASH [5% EtOAc in hexane] to afford 600 mg of the BOC-protected pyrrolyl-nitrotoluene. This material was reduced in EtOH:H₂O (4:1, 20 volumes), added Fe (20.0 eq), NH₄Cl (20.0 eq), reacted at 80° C. for 24 hours. The reaction was filtered on CELITE bed, filtrate was concentrated, and residue was washed with water and EtOAc (3×20 mL). The organic layer was dried over Na₂SO₄, filtered, concentrated, and purified by COMBIFLASH [40% EtOAc in hexane] to afford 400 mg of 3-methyl-5-(1H-pyrrol-2-yl)aniline (used for 1335 and 1330).

3-methyl-5-(pyrimidin-2-yl)aniline: Purged 1-bromo-3-methyl-5-nitrobenzene (2 g) in 1,4-dioxane (40 mL) with N₂ for 30 minutes, added bispinacalato diboron (1.5 eq), KOAc (3.0 eq), Pd(dppf)Cl₂ (0.1 eq) and mixed at 100° C. for 6 hours. The reaction mixture was concentrated and the residue dissolved in water (50 mL) and EtOAc (50 mL) and filtered over CELITE bed. The combined filtrates were extracted with EtOAc (2×50 mL). The organic layer was separated and concentrated to afford the boronate (2.0 g). Mixed the boronate (2.0 g) and 2-bromo-pyrimidine (1.5 eq) in EtOH:toluene:water (1:1:1, 40 mL), added Na₂CO₃ (3.0 eq), degassed N₂ 30 minutes, added Pd (dppf) Cl₂ (0.1 eq) and mixed 70° C. for 16 hours. Reaction mixture was distilled and the residue dissolved in water (50 mL) and EtOAc (50 mL) and filtered over CELITE bed. The combined filtrates were extracted with EtOAc (2×50 mL). The organic layer was dried over Na₂SO₄, filtered, concentrated, and purified by COMBIFLASH [20% EtOAc in hexane] providing 1 g of the pyrimidyl-nitro-toluene. This was reduced by mixing with EtOH/water (4:1) (15 mL), Fe (5.0 eq), NH₄Cl (5.0 eq) at 80° C. for 2 hours. The reaction mixture was distilled and the residue dissolved in EtOAc (20 mL) and filtered through CELITE bed and washed bed with EtOAc (2×10 mL). The combined filtrates were washed with water (10 mL), dried over anhydrous Na₂SO₄, filtered, concentrated, and purified by COMBIFLASH [40% EtOAc in hexane] providing 700 mg of 3-methyl-5-(pyrimidin-2-yl)aniline (used for 1319).

### Example 118: Synthesis of compounds 1322 ((2R,3R,4R,5S)-2-(hydroxymethyl)-1-{[6-({[3-methyl-5-(pyrimidin-2-yl)phenyl]amino}methyl)pyridin-2-yl]methyl}piperidine-3,4,5-triol), 1333 ((2R,3R,4R,5S)-1-{[6-({[3-cyclopropyl-5-(pyrimidin-2-yl)phenyl]amino}methyl)pyridin-2-yl]methyl}-2-(hydroxymethyl)piperidine-3,4,5-triol), 1334 ((2R,3R,4R,5S)-1-{[6-({[3-(furan-2-yl)-5-methylphenyl]amino}methyl)pyridin-2-yl]methyl}-2-(hydroxymethyl)piperidine-3,4,5-triol), 1335 ((2R,3R,4R,5S)-2-(hydroxymethyl)-1-{[6-({[3-methyl-5-(1H-pyrrol-2-yl)phenyl]amino}methyl)pyridin-2-yl]methyl}piperidine-3,4,5-triol), 1336 ((2R,3R,4R,5S)-2-(hydroxymethyl)-1-{[6-({[3-methyl-5-(morpholin-4-yl)phenyl]amino}methyl)pyridin-2-yl]methyl}piperidine-3,4,5-triol), and 1337 ((2R,3R,4R,5S)-2-(hydroxymethyl)-1-{[6-({[3-methyl-5-(1, 3-oxazol-2-yl)phenyl]amino}methyl)pyridin-2-yl]methyl}piperidine-3,4,5-triol)

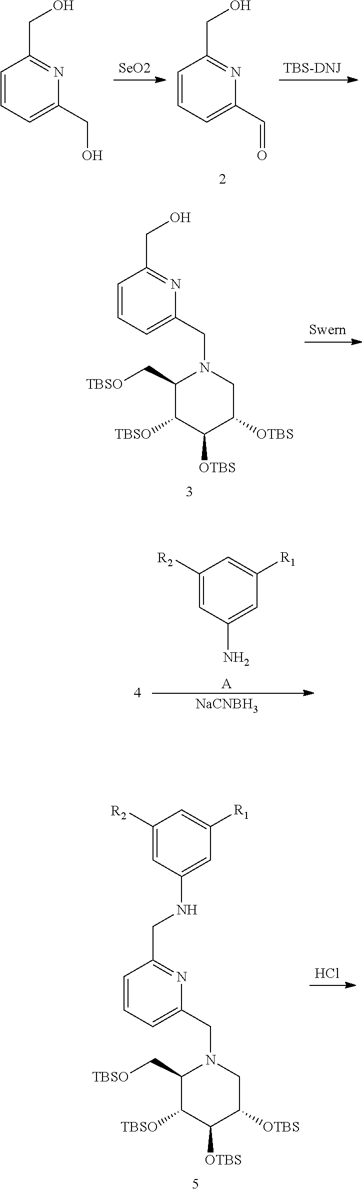

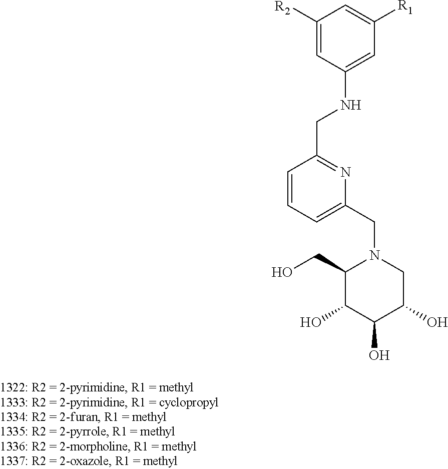

Pyridine-2,6-diyldimethanol (2.0 g), SeO₂ (0.6 eq), 1,4-dioxane (40 mL) reacted 16 hours at 65° C. Reaction was filtered through a bed of CELITE, filtrate evaporated, and residue purified by COMBIFLASH [3% EtOAc/hexane] giving 1.4 g of 2 as colorless syrup.

2 (1.4 g), DCM (5 mL), MeOH (30 mL), TBS-DNJ (0.5 eq), AcOH (cat.) mixed 10 minutes at room temperature, mixed NaCNBH₃ (1.5 eq) at 0° C. then 16 hours at room temperature. Reaction was concentrated and residue dissolved in EtOAc (50 mL) and washed with water (2×20 mL). The organic extract was dried over Na₂SO₄, filtered, concentrated, and purified by COMBIFLASH [15% EtOAc in hexane] to afford 2.5 g of 3.

To (COCl)₂ (3.0 eq) in DCM (30 mL) added DMSO (4.0 eq) at −78° C., mixed 30 minutes, added 3 (600 mg) in DCM (30 mL), stirred for 1 hour at −78° C., added Et₃N (5 eq) then bought to room temperature for 2 hours. Reaction was diluted with water (30 mL), extracted with DCM (2×30 mL), organic layer was washed with water and dried over anhydrous Na₂SO₄, filtered and concentrated to provide 500 mg of 4. Alternatively, this material was purified by COMBIFLASH [10% EtOAc/hexane] to give 200 mg of 4 as colorless syrup from 300 mg of 3.

Preparation of 1333 (2R,3R,4R,5S)-1-{[6-({[3-cyclopropyl-5-(pyrimidin-2-yl)phenyl]amino}methyl)pyridin-2-yl]methyl}-2-(hydroxymethyl)piperidine-3,4,5-triol: Mixed 4 (200 mg), 3-cyclopropyl-5-(pyrimidin-2-yl)aniline (1.0 eq, Example 10), DCM/MeOH (1:1, 20 mL), AcOH (cat.), NaCNBH₃ (1.5 eq) at room temperature for 16 hours then reduced volatiles. The residue was dissolved in EtOAc (15 mL), washed with water (2×10 mL), dried over Na₂SO₄, filtered, concentrated, and purified by COMBIFLASH [30% EtOAc in hexane] to afford 210 mg of 5.

Mixed 5 (225 mg), MeOH/DCM (1:1, 6 mL), 4.0 M HCl in dioxane (2.2 mL) at room temperature for 16 hours. The reaction was concentrated and the residue dissolved in water and extracted with EtOAc (10 mL). The aqueous layer was basified with solid NaHCO₃ and extracted with 10% MeOH/EtOAc (2×20 mL). Basic organic extracts were dried over Na₂SO₄, filtered and dried to obtain 36 mg of 1333.

Preparation of 1334 (2R,3R,4R,5S)-1-{[6-({[3-(furan-2-yl)-5-methylphenyl]amino}methyl)pyridin-2-yl]methyl}-2-(hydroxymethyl)piperidine-3,4,5-triol: Mixed 4 (130 mg), DCM (1 mL), MeOH (8 mL), 3-(furan-2-yl)-5-methylaniline (1.0 eq), AcOH (cat.) 10 minutes at room temperature, added NaCNBH₃ (1.5 eq) at 0° C. then reacted at room temperature for 16 hours. Volatiles are removed and the residue was diluted with water (7 mL) and extracted with EtOAc (2×10 mL). The organic layer was washed with water, dried over anhydrous Na₂SO₄, filtered, concentrated, and purified by COMBIFLASH [15% EtOAc/hexane] to obtain 90 mg of 5 as light yellow syrup.

Mixed 5 (110 mg), MeOH:DCM (1:1, 4 mL), 4.0 M HCl in 1,4-dioxane (1.4 mL) at 0° C. then at room temperature for 6 hours. Volatiles were removed and the residue washed with Et₂O (2×5 mL). The solid was diluted with water (2 mL) and washed with EtOAc (2×4 mL). The aqueous layer was basified with NaHCO₃ and extracted with 5% MeOH in EtOAc (3×6 mL). The organic layer was dried over anhydrous Na₂SO₄ and concentrated to afford 45 mg of 1334 as off white solid.

Preparation of 1335 (2R,3R,4R,5S)-2-(hydroxymethyl)-1-{[6-({[3-methyl-5-(1H-pyrrol-2-yl)phenyl]amino}methyl)pyridin-2-yl]methyl}piperidine-3,4,5-triol: Mixed 4 (240 mg), DCM (2 mL), MeOH (12 mL), 3-methyl-5-(1H-pyrrol-2-yl)aniline (1.0 eq) and AcOH (cat.) 10 minutes at room temperature, added NaCNBH₃ (1.5 eq) at 0° C., increased to room temperature for 16 hours. Volatiles are removed and the residue was diluted with water (10 mL) and extracted with EtOAc (2×20 mL). The organic layer was washed with water and dried over anhydrous Na₂SO₄, filtered, concentrated, and purified by COMBIFLASH [15% EtOAc-hexane] to provide 150 mg of 5 as light yellow syrup.

5 (150 mg), MeOH:DCM (1:1, 6 mL), 4.0 M HCl in 1,4-dioxane (2.0 mL) mixed at 0° C. then at room temperature for 6 hours. Volatiles are removed and the residue was washed with Et₂O (2×5 mL). The material was diluted with water (5 mL) and washed with EtOAc (2×10 mL). The aqueous layer was basified with NaHCO₃ and extracted with 5% MeOH in EtOAc (5×10 mL). Combined basic organic extract was dried over anhydrous Na₂SO₄ and concentrated to afford 25 mg of 1335.

Preparation of 1336 (2R,3R,4R,5S)-2-(hydroxymethyl)-1-{[6-({[3-methyl-5-(morpholin-4-yl)phenyl]amino}methyl)pyridin-2-yl]methyl}piperidine-3,4,5-triol: Mixed 4 (130 mg), DCM (1 mL), MeOH (8 mL), 3-methyl-5-morpholinoaniline (1.0 eq), AcOH (cat.) 10 minutes at room temperature, added NaCNBH₃ (1.5 eq) 0° C. then reacted at room temperature 16 hours. Volatiles were removed and the residue was diluted with water (7 mL) and extracted with EtOAc (2×10 mL). The organic layer was washed with water, dried over anhydrous Na₂SO₄, filtered, concentrated, and purified by COMBIFLASH [15% EtOAc-hexane] to give 90 mg of 5 as light yellow syrup.

Mixed 5 (110 mg), MeOH:DCM (1:1, 4 mL), 4.0 M HCl in 1,4-dioxane (1.4 mL) at 0° C. then at room temperature for 6 hours. Volatiles were removed and the residue washed with Et₂O (2×5 mL). The material was diluted with water (2 mL) and washed with EtOAc (2×4 mL). The aqueous layer was basified with NaHCO₃ and extracted with 5% MeOH in EtOAc (3×6 mL). Combined organic layer was dried over anhydrous Na₂SO₄ and concentrated to afford 38 mg of 1336 as off white solid.

Preparation of 1337 (2R,3R,4R,5S)-2-(hydroxymethyl)-1-{[6-({[3-methyl-5-(1,3-oxazol-2-yl)phenyl]amino}methyl)pyridin-2-yl]methyl}piperidine-3,4,5-triol: Mixed 4 (180 mg), 3-methyl-5-(oxazol-2-yl)aniline (1.0 eq), DCM/MeOH (1:1, 20 mL), AcOH (cat.) and NaCNBH₃ (1.5 eq) at room temperature 16 hours. Reaction was concentrated and the residue was dissolved in EtOAc (15 mL) and washed with water (2×10 mL), dried over Na₂SO₄, then filtered, concentrated, then purified by COMBIFLASH [30% EtOAc in hexane] to afford 100 mg of 5.

5 (100 mg, 0.11 mmol), MeOH/DCM (1:1) (4 mL), 4 M HCl in Dioxane (1.1 mL), room temperature, 16 hours. Concentrated and residue purified by preparative HPLC gave 4 mg of 1337 after lyophilization.

Preparation of 1322 (2R,3R,4R,5S)-2-(hydroxymethyl)-1-{[6-({[3-methyl-5-(pyrimidin-2-yl)phenyl]amino}methyl)pyridin-2-yl]methyl}piperidine-3,4,5-triol: 1322 was prepared by a variation of the indicated scheme in which coupling of the aryl ring was carried out prior to that of the protected DNJ (intermediates not shown on figure). Mixed 2 (1.0 g), MeOH (30 mL), 3-bromo-5-methylaniline (1.0 eq), AcOH (cat.) 10 minutes at room temperature, added NaCNBH₃ (1.5 eq) at 0° C. then mixed 16 hours at room temperature. Volatiles were removed, residue was diluted with water (10 mL) and extracted with EtOAc (2×15 mL). The organic layer was washed with water, dried over anhydrous Na₂SO₄, filtered, concentrated, and purified by COMBIFLASH [5-20% EtOAc/hexane] to afford 1.0 g of aryl-coupled product.

SeO₂ (0.5 eq) and 700 mg of the aryl-coupled intermediate in 1,4-dioxane (15 mL) were reacted 16 hours at 50° C. Reaction was filtered through a CELITE bed and washed with EtOAc (3×20 mL). The filtrate was concentrated and purified by COMBIFLASH [10%-20% EtOAc in hexane] to afford 310 mg of the aldehyde.

Mixed the aldehyde (300 mg), MeOH (15 mL), TBS-DNJ (0.8 eq), AcOH (cat.) 10 minutes at room temperature, added NaCNBH₃ (1.5 eq), reacted at room temperature 16 hours. Removed volatiles, diluted residue with water (10 mL), extracted with EtOAc (2×15 mL). The organic layer was washed with water, dried over anhydrous Na₂SO₄, filtered, concentrated, and purified by COMBIFLASH [5-20% EtOAc/hexane] to give 295 mg of TBS-DNJ-coupled intermediate.

Degassed TBS-DNJ-coupled intermediate (290 mg), 1,4-dioxane (10 mL), bis(pinacalato) diboron (1.5 eq), KOAc (3.0 eq) with N₂ for 20 minutes, added Pd(dppf)Cl₂ (0.1 eq), heated at 100° C. 16 hours. The reaction was incomplete so worked up and added the same equivalents of the diboronate, KOAc, and the palladium salt and re-subjected to the same reaction conditions. Solvent was removed, residue was diluted with water and extracted with EtOAc (2×20 mL), combined organic layer was dried over anhydrous Na₂SO₄ and concentrated to afford 297 mg of the boronate.

Boronate (290 mg), 2-bromopyrimidine (1.5 eq), toluene:EtOH:water (1:1:1, 12 mL) and Na₂CO₃ (3.0 eq) were degassed 20 minutes with N₂, added Pd(dppf)Cl₂ (0.1 eq), reacted at 100° C. 6 hours. Reaction was cooled to room temperature, volatiles removed, residue diluted with water and EtOAc and filtered through CELITE bed, filtrate was extracted with EtOAc (3×200 mL). Organic layer was dried over anhydrous Na₂SO₄, filtered, concentrated, and purified by COMBIFLASH [1020 EtOAc:hexane] to afford 70 mg of 5.

Mixed 5 (70 mg), MeOH:DCM (1:1, 3 mL), 4.0 M HCl in 1,4-dioxane (0.7 mL) at 0° C. then at room temperature for 6 hours. Reaction was concentrated, residue was suspended in water (5 mL) and washed with EtOAc (3×3 mL). The aqueous layer was adjusted to pH 8 with saturated NaHCO₃ solution and extracted with EtOAc (3×5 mL). The combined organic extracts were concentrated then lyophilized to obtain 7 mg of 1322.

Table 2, below, includes the NMR and MS data from compounds disclosed in Examples 1-118.

| | Calc. | Obs. | |
|---|---|---|---|
| # | Mass | Mass | Proton NMR (Solvent and Peak List) |
| 1001 | 490.6 | 491.5 | (400 MHZ, DMSO-d₆): δ 10.08 (brs, 1H), 9.24 (d, 1H), 8.30 (d, 1H), |
| | | (M + H) | 7.88 (dd, 1H), 7.50 (d, 2H), 7.35 (d, 3H), 7.10 (brs, 1H), 6.70 (brs, 1H), |
| | | | 4.81-4.63 (m, 3H), 4.17-4.06 (m, 3H), 3.59-3.49 (m, 1H), 3.47-3.42 (m, |
| | | | 1H), 3.18-3.06 (m, 4H), 3.00-2.95 (m, 1H), 2.87-2.78 (m, 1H), 2.74-2.60 |
| | | | (m, 1H), 1.99-1.87 (m, 1H), 0.99-0.89 (m, 2H), 0.74-0.63 (m, 2H) |
| 1003 | 451.6 | 452.4 | (400 MHZ, CD₃OD): δ 6.44 (s, 2H), 6.39 (s, 1H), 3.86 (t, 2H), 3.72-3.59 |
| | | (M + H) | (m, 4H), 3.51-3.45 (m, 1H), 3.40-3.33 (m, 3H), 3.18-3.11 (m, 1H), 3.09- |
| | | | 2.99 (m, 3H), 2.88-2.78 (m, 1H), 2.66-2.56 (m, 1H), 2.51-2.42 (m, 4H), |
| | | | 2.27-2.12 (m, 5H), 1.65-1.58 (m, 2H), 1.57-1.41 (m, 4H), 1.40-1.27 (m, |
| | | | 2H) |
| 1005 | 471.6 | 472.5 | (400 MHZ, DMSO-d₆): δ 11.21 (brs, 1H), 10.27 (brs, 1H), 7.61-7.35 (m, |
| | | (M + H) | 4H), 6.79 (brs, 1H), 6.66-6.54 (m, 2H), 4.82-4.64 (m, 2H), 4.4-4.6(3H), |
| | | | 4.36-4.34 (m, 2H), 4.15-4.00 (m, 5H), 3.92-3.88 (m, 2H), 3.84-3.78 (m, |
| | | | 2H), 3.59-3.52 (m, 1H), 3.51-3.45 (m, 1H), 3.16-3.10 (m, 3H), 3.03-2.92 |
| | | | (m, 3H), 2.80-2.77 (m, 1H), 2.71-2.61 (m, 1H), 2.18 (s, 3H) |
| 1006 | 463.6 | 464.5 | (400 MHZ, CD₃OD): δ 6.08-6.05 (m, 2H), 5.98-5.95 (m, 1H), 3.89-3.76 |
| | | (M + H) | (m, 6H), 3.53-3.44 (m, 1H), 3.37-3.32 (m, 1H), 3.20-2.98 (m, 8H), 2.87- |
| | | | 2.77 (m, 1H), 2.61-2.51 (m, 1H), 2.24-2.05 (m, 2H), 1.80-1.75 (m, 1H), |
| | | | 1.66-1.55 (m, 2H), 1.54-1.47 (m, 2H), 1.47-1.39 (m, 2H), 1.38-1.26 (m, |
| | | | 2H), 0.92-0.82 (m, 2H), 0.67-0.55 (m, 2H) |
| 1008 | 456.5 | 457.5 | (400 MHZ, CD₃OD): δ 9.59 (dd, 1H), 9.02 (dd, 1H), 8.53 (dd, 1H), 8.17 |
| | | (M + H) | (t, 1H), 8.02 (s, 1H), 7.55 (s, 1H), 4.14-4.10 (m, 1H), 3.95-3.91 (m, 1H), |
| | | | 3.75-3.62 (m, 1H), 3.65-3.57 (m, 1H), 3.55-3.44 (m, 3H), 3.43-3.36 (m, |
| | | | 2H), 3.27-3.20 (m, 1H), 3.11-2.92 (m, 2H), 2.23-2.12 (m, 1H), 1.93-1.74 |
| | | | (m, 4H), 1.64-1.43 (m, 4H), 1.26-1.15 (m, 2H), 0.97-0.89 (m, 2H) |
| 1009 | 456.59 | 457.5 | (400 MHZ, CD₃OD): δ 8.80 (d, 2H), 7.46-7.41 (m, 1H), 7.36 (t, 1H), |
| | | (M + H) | 7.33 (t, 1H), 6.56 (t, 1H), 3.93-3.81 (m, 2H), 3.49-3.44 (m, 1H), 3.39- |
| | | | 3.31 (m, 1H), 3.20-3.09 (m, 3H), 3.01-2.97 (m, 1H), 2.88-2.75 (m, 1H), |
| | | | 2.63-2.51 (m, 1H), 2.16 (t, 1H), 2.12-2.08 (m, 1H), 1.93-1.88 (m, 1H), |
| | | | 1.70-1.63 (m, 2H), 1.58-1.43 (m, 4H), 1.41-1.32 (m, 2H), 1.03-0.86 (m, |
| | | | 2H), 0.75-0.69 (m, 2H) |
| 1010 | 483.61 | 484.5 | (400 MHZ, D₂O): δ 6.80 (d, J = 8.07 Hz, 2H), 6.75 (d, J = 8.8 Hz, 2H), |
| | | (M + H) | 6.67 (d, J = 8.1 Hz, 2H), 6.33 (s, 1H), 4.12-4.09 (m, 1H), 3.92 (s, 2H), |
| | | | 3.56-3.54 (m, 2H), 3.49-3.39 (m, 5H), 3.10-2.99 (m, 2H), 2.98-2.94 (m, |
| | | | 4H), 2.84-2.75 (m, 1H), 2.59-2.45 (m, 2H), 2.24-2.06 (m, 1H), 1.30-1.20 |
| | | | (m, 1H), 0.42-0.32 (m, 2H), -0.05- - 0.16 (m, 2H). |
| 1012 | 476.58 | 477.5 | (400 MHZ, CD₃OD): δ 9.50 (dd, 1H), 8.86 (dd, 1H), 8.45 (dd, 1H), 7.72- |
| | | (M + H) | 7.51 (m, 6H), 7.08 (s, 1H), 4.66 (s, 2H), 4.33-4.13 (m, 3H), 3.68-3.52 (m, |
| | | | 3H), 3.36-3.33 (m, 1H), 3.18-3.01 (m, 2H), 2.94-2.67 (m, 1H), 2.08-1.92 |
| | | | (m, 1H), 1.12-1.05 (m, 2H), 0.84-0.76 (m, 2H) |
| 1013 | 476.58 | 477.5 | (400 MHZ, CD₃OD): δ 8.90 (d, 2H), 8.23 (d, 2H), 7.62 (s, 4H), 7.46 |
| | | (M + H) | (t, 1H), 7.27 (t, 1H), 4.86-4.84 (m, 1H), 4.74 (s, 2H), 4.33-4.14 (m, 3H), |
| | | | 3.65-3.52 (m, 2H), 3.38-3.32 (m, 1H), 3.19-3.04 (m, 2H), 2.89-2.83 (m, |
| | | | 1H), 2.11-2.05 (m, 1H), 1.14-1.05 (m, 2H), 0.85-0.70 (m, 2H) |
| 1014 | 516.6 | 517.60 | (400 MHZ, CD₃OD): δ 8.37 (d, 1H), 7.70 (dd, 1H), 7.54 (s, 4H), 7.07 (d, |
| | | (M + H) | 1H), 4.86-4.83 (m, 1H), 4.73 (s, 2H), 4.29-4.13 (m, 3H), 4.12-4.01 (m, |
| | | | 2H), 3.69-3.51 (m, 4H), 3.38-3.32 (m, 1H), 3.18-3.03 (m, 4H), 2.88-2.82 |
| | | | (m, 1H), 1.27 (d, 6H) |
| 1015 | 500.55 | 501.6 | (500 MHZ, DMSO-d₆): δ 10.05-10.00 (m, 1H), 8.47-8.35 (m, 1H), 7.53- |
| | | (M + H) | 7.49 (m, 2H), 7.46-7.43 (m, 2H), 7.18-7.15 (m, 1H), 7.12-7.09 (m, 1H), |
| | | | 6.88-6.86 (m, 1H), 4.72-4.68 (m, 2H), 4.63 (s, 2H), 4.59-4.44 (m, 2H), |
| | | | 4.18-3.99 (m, 1H), 3.71-3.69 (m, 1H), 3.62-3.59 (m, 1H), 3.56-3.41 (m, |
| | | | 4H), 3.12 (t, 1H), 3.01-2.97 (m, 1H), 2.89-2.86 (m, 1H), 2.81-2.72 (m, |
| | | | 2H), 2.72-2.63 (m, 2H), 2.55-2.53 (m, 1H), 1.99-1.73 (m, 2H) |
| 1018 | 514.58 | 515.8 | (400 MHZ, CD₃OD): δ 7.57 (d, 1H), 7.35 (s, 4H), 7.21 (dd, 1H), 6.91 (d, |
| | | (M + H) | 1H), 4.57 (s, 2H), 4.26-4.23 (m, 1H), 4.10-4.07 (m, 1H), 3.98-3.96 (m, |
| | | | 2H), 3.95-3.90 (m, 1H), 3.86-3.81 (m, 2H), 3.58-3.53 (m, 2H), 3.43-3.34 |
| | | | (m, 2H), 3.28-3.24 (m, 1H), 3.17-3.09 (m, 1H), 2.87-2.83 (m, 1H), 2.15- |
| | | | 2.11 (m, 1H), 2.03-1.84 (m, 5H) |
| 1020 | 502.57 | 503.3 | (400 MHZ, CD₃OD): δ 7.73 (d, 1H), 7.44 (dd, 1H), 7.30-7.14 (m, 5H), |
| | | (M + H) | 4.36 (s, 2H), 4.25-4.23 (m, 1H), 4.09-4.05 (m, 2H), 3.95-3.80 (m, 4H), |
| | | | 3.45-3.32 (m, 3H), 3.23-3.03 (m, 2H), 2.92-2.78 (m, 5H), 2.37-2.23 (m, |
| | | | 2H), 2.19-2.16 (m, 1H), 1.96-1.87 (m, 1H) |
| 1021 | 451.36 | 451.3 | (400 MHZ, CD₃OD): δ 7.67 (s, 1H), 7.57-7.54 (m, 3H), 7.24 (s, 1H), |
| | | (M+) | 7.19 (s, 1H), 7.04 (s, 1H), 4.91-4.83 (m, 1H), 4.58 (s, 2H), 4.28-4.14 (m, |
| | | | 3H), 3.72-3.53 (m, 2H), 3.41-3.29 (m, 1H), 3.22-3.03 (m, 2H), 2.85 (t, |
| | | | 1H), 2.33 (s, 3H) |
| 1022 | 515.42 | 515.3 | (400 MHZ, CD₃OD): δ 7.56-7.41 (m, 4H), 7.21 (t, , 1H), 7.05-7.00 (m, |
| | | (M+) | 2H), 4.84-4.76 (m, 1H), 4.46 (s, 2H), 4.29-4.09 (m, 3H), 3.71-3.54 (m, |
| | | | 2H), 3.35-3.30 (m, 1H), 3.16-3.12 (m, 1H), 3.10-3.07 (m, 1H), 3.06 (s, |
| | | | 3H), 2.85-2.79 (m, 1H) |
| 1023 | 462.34 | 462.34 | (400 MHZ, CD₃OD): δ 7.56-7.48 (m, 3H), 7.46-7.42 (m, 1H), 7.05-6.96 |
| | | (M+) | (m, 2H), 6.81 (s, 1H), 4.42 (s, 2H), 4.29-4.03 (m, 4H), 3.68-3.53 (m, 2H), |
| | | | 3.36-3.34 (m, 1H), 3.20-3.02 (m, 2H), 2.82 (t, 1H). |
| 1024 | 451.36 | 451.3 | (400 MHz, CD₃OD): δ 7.33-7.30 (m, 4H), 6.53 (s, 2H), 6.38 (s, 1H), |
| | | (M+) | 4.31-4.18 (m, 3H), 4.09 (dd, 1H), 3.93 (dd, 1H), 3.43 -3.33 (m, 2H), 3.26 |
| | | | (d, 1H), 3.17-3.07 (m, 1H), 2.86 (dd, 1H), 2.21-2.05 (m, 4H), 1.88 (t, 1H) |
| 1025 | 515.42 | 515.3 | (400 MHZ, CD₃OD): δ 7.39-7.35 (m, 4H), 7.18 (s, 1H), 7.05 (s, 1H), |
| | | (M+) | 7.00 (s, 1H), 4.38-4.27 (m, 3H), 4.10 (dd, 1H), 3.96 (dd, 1H), 3.46-3.35 |
| | | | (m, 3H), 3.19-3.09 (m, 1H), 3.03 (s, 3H), 2.88 (dd, 1H), 2.32-2.15 (m, |
| | | | 1H), 2.09-1.85 (m, 1H) |
| 1026 | 462.34 | 462.3 | (400 MHZ, CD₃OD): δ7.38-7.26 (m, 4H), 7.02-6.97 (m, 2H), 6.83-6.80 |
| | | (M+) | (m, 1H), 4.38 - 4.22 (m, 3H), 4.09 (dd, 1H), 3.95 (dd, 1H), 3.49-3.34 (m, |
| | | | 3H), 3.21-3.05 (m, 1H), 2.88 (dd, 1H), 2.30-2.12 (m, 1H), 2.05-1.86 (m, |
| | | | 1H) |
| 1027 | 437.33 | 437.3 | (400 MHZ, CD₃OD): δ 7.35 (s, 1H), 7.30-7.19 (m, 3H), 7.14 (d, 2H), |
| | | (M+) | 6.52 (d, 2H), 4.36-4.17 (m, 3H), 4.07 (dd, 1H), 3.92 (dd, 1H), 3.44-3.33 |
| | | | (m, 2H), 3.26 (d, 1H), 3.12 (t, 1H), 2.85 (dd, 1H), 2.16-2.10 (m, 1H), 1.89 |
| | | | (t, 1H) |
| 1028 | 437.33 | 437.0 | (400 MHZ, CD₃OD): δ7.41-7.22 (m, 4H), 7.14 (d, 2H), 6.52 (d, 2H), |
| | | (M+) | 4.27 (s, 2H), 4.22 (d, 1H), 4.08 (dd, 1H), 3.92 (dd, 1H), 3.44 - 3.33 (m, |
| | | | 2H), 3.25 (d, 1H), 3.16 - 3.05 (m, 1H), 2.85 (dd, 1H), 2.15-2.09 (m, 1H), |
| | | | 1.87 (t, 1H) |
| 1029 | 482.33 | 482 | (400 MHZ, CD₃OD): δ 8.27 (d, 1H), 7.50 (dd, 1H), 7.39 (s, 1H), 7.35- |
| | | (M+) | 7.24 (m, 3H), 6.88 (d, 1H), 4.60 (s, 2H), 4.25 (d, 1H), 4.06 (dd, 1H), 3.90 |
| | | | (dd, 1H), 3.43-3.33 (m, 2H), 3.26 (d, 1H), 3.15-3.07 (m, 1H), 2.82 (dd, |
| | | | 1H), 2.14-2.09 (m, 1H), 1.87 (t, 1H) |
| 1030 | 482.33 | 482 | (400 MHZ, CD₃OD): δ 8.27 (d, 1H), 7.49 (dd, 1H), 7.40-7.29 (m, 4H), |
| | | (M+) | 6.89 (d, 1H), 4.59 (s, 2H), 4.25 (d, 1H), 4.08 (dd, 1H), 3.93 (dd, 1H), |
| | | | 3.44-3.33 (m, 2H), 3.27 (d, 1H), 3.16-3.09 (m, 1H), 2.85 (dd, 1H), 2.14 |
| | | | (d, 1H), 1.89 (t, 1H) |
| 1031 | 457.57 | 458.5 | (400 MHZ, D₂O + TFA): δ 7.55-7.42 (m, 3H), 7.40-7.32 (m, 2H), 7.22 (d, |
| | | (M + H) | 2H), 4.62 (s, 2H), 4.19-4.06 (m, 4H), 4.06-4.00 (m, 4H), 3.69- 3.52 (m, |
| | | | 6H), 3.47-3.34 (m, 1H), 3.20-3.06 (m, 2H), 2.80 (t, 1H), 2.34 (s, 3H) |
| 1032 | 456.54 | 457.5 | (400 MHZ, CD₃OD): δ 7.36 (s, 1H), 7.29-7.17 (m, 3H), 6.54 (s, 1H), |
| | | (M + H) | 6.53-6.48 (m, 2H), 6.34 (s, 1H), 4.28 (s, 2H), 4.22 (d, 1H), 4.19-4.15 (m, |
| | | | 2H), 4.10-4.03 (m, 3H), 3.91 (dd, 1H), 3.44-3.33 (m, 2H), 3.26 (d, 1H), |
| | | | 3.17-3.07 (m, 1H), 2.85 (dd, 1H), 2.18 (s, 3H), 2.14-2.08 (m, 1H), 1.92- |
| | | | 1.84 (m, 1H) |
| 1033 | 437.54 | 438.5 | (400 MHZ, CD₃OD): δ7.39 (s, 1H), 7.31-7.24 (m, 2H), 7.23-7.19 (m, |
| | | (M + H) | 1H), 6.75-6.67 (m, 3H), 6.34-6.30 (m, 2H), 6.10 (dd, 1H), 4.32 (s, 2H), |
| | | | 4.22 (d, 1H), 4.06 (dd, 1H), 3.91 (dd, 1H), 3.44 - 3.33 (m, 2H), 3.25 (d, |
| | | | 1H), 3.12 (t, 1H), 2.86 (dd, 1H), 2.22 (s, 3H), 2.14-2.09 (m, 1H), 1.88 (t, |
| | | | 1H) |
| 1034 | 450.54 | 451.4 | (400 MHZ, Acetic acid-d₄): δ 9.25 (d, 1H), 8.15 (dd, 1H), 7.88 (dd, 1H), |
| | | (M + H) | 7.65 (s, 1H), 7.58-7.49 (m, 1H), 7.48- 7.39 (m, 2H), 7.33 (s, 1H), 7.22 (s, |
| | | | 1H), 6.83 (s, 1H), 4.86 (d, 1H), 4.53 (s, 2H), 4.41-4.24 (m, 3H), 4.16 - |
| | | | 3.92 (m, 2H), 3.59 (t, 1H), 3.41 (dd, 1H), 3.27 (d, 1H), 2.91 (br t, 1H), |
| | | | 2.35 (s, 3H) |
| 1035 | 450.54 | 451.4 | (400 MHZ, CD₃OD): δ 8.92 (d, 2H), 8.36 (s, 1H), 8.27 (s, 1H), 7.74 (s, |
| | | (M + H) | 1H), 7.65-7.54 (m, 3H), 7.50-7.46 (m, 2H), 4.86-4.83 (m, 1H), 4.75 (d, |
| | | | 2H), 4.31-4.12 (m, 3H), 3.72-3.54 (m, 2H), 3.37-3.32 (m, 1H), 3.20-3.06 |
| | | | (m, 2H), 2.85 (t, 1H), 2.51 (s, 3H) |
| 1036 | 521.63 | 522.5 | (400 MHz, CD₃OD): δ 7.41 (s, 1H), 7.34-7.23 (m, 3H), 6.70 (s, 1H), |
| | | (M + H) | 6.63 (s, 1H), 6.40 (s, 1H), 4.45-4.27 (m, 3H), 4.12-4.05 (m, 1H), 4.01- |
| | | | 3.92 (m, 1H), 3.84-3.75 (m, 4H), 3.51-3.36 (m, 3H), 3.19-3.07 (m, 5H), |
| | | | 3.01 (s, 3H), 2.89 (dd, 1H), 2.52-2.18 (m, 1H), 2.12-1.94 (m, 1H) |
| 1040 | 514.6 | 515.5 | (400 MHZ, CD₃OD): δ 9.60 (d, 1H), 9.00 (d, 1H), 8.58 (dd, 1H), 7.86 (s, |
| | | (M + H) | 1H), 7.69 (s, 1H), 7.65-7.56 (m, 2H), 7.50 (t, 1H), 7.47-7.41 (m, 1H), |
| | | | 7.38 (t, 1H), 4.86-4.83 (m, 1H), 4.60 (s, 2H), 4.25-4.11 (m, 3H), 3.67- |
| | | | 3.51 (m, 2H), 3.29-3.24 (m, 1H), 3.17-3.05 (m, 5H), 2.80 (t, 1H) |
| 1041 | 514.6 | 515.5 | (400 MHZ, CD₃OD): δ 8.95 (d, 2H), 8.30 (s, 1H), 8.12 (s, 1H), 7.66-7.39 |
| | | (M + H) | (m, 6H), 4.86-4.88 (m, 1H), 4.62 (s, 2H), 4.28-4.10 (m, 3H), 3.69-3.51 |
| | | | (m, 2H), 3.35-3.33 (m, 0.5H), 3.29 - 3.25 (m, 0.5H), 3.18-3.07 (m, 5H), |
| | | | 2.83 (t, 1H) |
| 1042 | 468.55 | 469.6 | (400 MHZ, CD₃OD): δ 7.36 (s, 1H), 7.31-7.19 (m, 3H), 6.50 (s, 1H), |
| | | (M + H) | 6.42-6.36 (m, 2H), 4.32 (s, 2H), 4.23 (d, 1H), 4.07 (dd, 1H), 3.91 (dd, |
| | | | 1H), 3.82-3.73 (m, 4H), 3.43-3.33 (m, 3H), 3.26 (d, 1H), 3.16 - 3.04 (m, |
| | | | 4H), 2.84 (dd, 1H), 2.11 (d, 1H), 1.88 (t, 1H) |
| 1044 | 467.52 | 468.3 | (400 MHZ, CD₃OD): δ 7.36 (s, 1H), 7.32-7.22 (m, 3H), 6.94-6.91 (m, |
| | | (M + H) | 2H), 6.71 (s, 1H), 6.67 (dd, 1H), 4.32 (s, 2H), 4.27-4.18 (m, 3H), 4.11- |
| | | | 4.04 (m, 3H), 3.91 (dd, 1H), 3.43-3.32 (m, 2H), 3.26 (d, 1H), 3.17-3.08 |
| | | | (m, 1H), 2.85 (dd, 1H), 2.15-2.09 (m, 1H), 1.96-1.84 (m, 1H) |
| 1045 | 448.52 | 449.4 | (400 MHZ, CD₃OD): δ 7.40 (s, 1H), 7.33-7.22 (m, 3H), 7.11-7.05 (m, |
| | | (M + H) | 2H), 6.82-6.79 (m, 1H), 6.67-6.64 (m, 1H), 6.44 (dd, 1H), 6.17-6.12 (m, |
| | | | 1H), 4.37 (s, 2H), 4.25 (d, 1H), 4.07 (dd, 1H), 3.92 (dd, 1H), 3.41-3.34 |
| | | | (m, 2H), 3.26 (d,1H), 3.16-3.07 (m, 1H), 2.89-2.83 (m, 1H), 2.15-2.09 |
| | | | (m, 1H), 1.93-1.84 (m, 1H). |
| 1046 | 461.52 | 462.3 | (400 MHZ, Acetic acid): δ 9.32 (br d, 1H), 8.24 (br d, 1H), 7.93 (br dd, |
| | | (M + H) | 1H), 7.81-7.69 (m, 2H), 7.61-7.52 (m, 2H), 7.47-7.45 (m, 2H), 7.12 (s, |
| | | | 1H), 4.96 (br 1H), 4.54 (s, 2H), 4.36 (br s, 2H), 4.33-4.23 (m, 1H), 4.20- |
| | | | 4.09 (m, 1H), 4.05 (br t, 1H), 3.62 (br t, 1H), 3.41-3.27 (m, 2H), 2.96- |
| | | | 2.90 (m, 1H). |
| 1047 | 461.52 | 462.2 | (400 MHZ, CD₃OD): δ 8.84 (d, 2H), 7.98 (dd, 1H), 7.91 (t, 1H), 7.42 (s, |
| | | (M + H) | 1H), 7.37 (t, J = 4.9 Hz, 1H), 7.33- 7.22 (m, 3H), 6.97 (dd, 1H), 4.42 (s, |
| | | | 2H), 4.25 (d, 1H), 4.07 (dd, 1H), 3.91 (dd, 1H), 3.40-3.34 (m, 2H), 3.17 - |
| | | | 3.05 (m, 1H), 2.86 (dd, 1H), 2.13 (d, 1H), 1.89 (t, 1H). |
| 1048 | 457.57 | 458.2 | (400 MHZ, CD₃OD): δ 7.39-7.23 (m, 4H), 6.13-6.04 (m, 3H), 4.27 (s, |
| | | (M + H) | 2H), 4.21 (d, 1H), 4.08 (dd, 1H), 3.92 (dd, 1H), 3.79-3.75 (m, 4H), 3.43- |
| | | | 3.33 (m, 2H), 3.26 (d, 1H), 3.15-3.08 (m, 1H), 3.04-2.98 (m, 4H), 2.88- |
| | | | 2.81 (m, 1H), 2.18-2.07 (m, 4H), 1.91-1.82 (m, 1H) |
| 1050 | 456.54 | 457.3 | (500 MHZ, CD₃OD): δ 7.37-7.27 (m, 4H), 6.56-6.50 (m, 3H), 6.35 (s, |
| | | (M + H) | 1H), 4.29 (s, 2H), 4.24 (d, 1H), 4.20-4.15 (m, 2H), 4.12-4.05 (m, 3H), |
| | | | 3.94 (dd, 1H), 3.44-3.35 (m, 2H), 3.28 (d, 1H), 3.13 (t, 1H), 2.88 (dd, |
| | | | 1H), 2.18 (s, 3H), 2.17-2.12 (m, 1H), 1.96-1.87 (m, 1H) |
| 1051 | 437.54 | 438.3 | (400 MHZ, CD₃OD): δ 7.39-7.28 (m, 4H), 6.77-6.65 (m, 3H), 6.36-6.27 |
| | | (M + H) | (m, 2H), 6.09 (t, 1H), 4.32 (s, 2H), 4.22 (d, 1H), 4.08 (dd, 1H), 3.93 (dd, |
| | | | 1H), 3.43-3.35 (m, 2H), 3.26 (d, 1H), 3.12 (t, 1H), 2.87 (dd, 1H), 2.22 (s, |
| | | | 3H), 2.16-2.10 (m, 1H), 1.94-1.84 (m, 1H) |
| 1052 | 450.54 | 451.8 | (400 MHZ, CD₃OD): δ9.08 (dd, 1H), 8.00 (dd, 1H), 7.72 (dd, 1H), 7.49- |
| | | (M + H) | 7.30 (m, 4H), 7.08 (d, 2H), 6.64 (s, 1H), 4.51-4.25 (m, 3H), 4.14-4.06 (m, |
| | | | 1H), 4.02-3.97 (m, 1H), 3.57-3.34 (m, 3H), 3.23-3.13 (m, 1H), 2.93 (dd, |
| | | | 1H), 2.42-2.26 (m, 4H), 2.19-2.02 (m, 1H) |
| 1053 | 450.54 | 451.3 | (400 MHZ, CD₃OD): δ 8.77 (d, 2H), 7.49-7.27 (m, 7H), 6.62 (s, 1H), |
| | | (M + H) | 4.43-4.23 (m, 3H), 4.09 (dd, 1H), 3.99-3.96 ( m, 1H), 3.48-3.33 (m, 3H), |
| | | | 3.14 (t, 1H), 2.922.89 (m, 1H), 2.49-1.86 (m, 5H) |
| 1054 | 521.63 | 522.5 | (400 MHZ, CD₃OD): δ7.39-7.25 (m, 4H), 6.72-6.67 (m, 1H), 6.63 (t, |
| | | (M + H) | 1H), 6.39 (t, 1H), 4.35 (s, 2H), 4.22 (d, 1H), 4.08 (dd, 1H), 3.92 (dd, 1H), |
| | | | 3.85-3.73 (m, 4H), 3.43-3.33 (m, 2H), 3.26 (d, 1H), 3.15 - 3.07 (m, 5H), |
| | | | 3.00 (s, 3H), 2.83 (dd, 1H), 2.14-2.09 (m, 1H), 1.86 (t, 1H) |
| 1058 | 514.6 | 515.5 | (400 MHZ, CD₃OD): δ 9.62 (d, 1H), 9.01 (d, 1H), 8.61 (dd, 1H), 7.85 (s, |
| | | (M + H) | 1H), 7.69 (s, 1H), 7.61-7.52 (m, 4H), 7.35 (s, 1H), 4.83 (d, 1H), 4.59 (s, |
| | | | 2H), 4.32-4.11 (m, 3H), 3.70-3.49 (m, 2H), 3.37-3.33 (m, 1H), 3.17-3.07 |
| | | | (m, 5H), 2.84 (t, 1H) |
| 1059 | 514.6 | 515.5 | (400 MHZ, CD₃OD): δ 8.94 (d, 2H), 8.30 (s, 1H), 8.13 (s, 1H), 7.63- |
| | | (M + H) | 7.48 (m, 5H), 7.38 (s, 1H), 4.83 (d, 1H), 4.61 (s, 2H), 4.30-4.08 (m, 3H), |
| | | | 3.69-3.49 (m, 2H), 3.34-3.32 (m, 1H), 3.18-3.07 (m, 5H), 2.84 (t, 1H) |
| 1060 | 468.55 | 469.4 | (400 MHZ, CD₃OD): δ 7.33-7.32 (m, 4H), 6.49 (s, 1H), 6.40-6.37(m, |
| | | (M + H) | 2H), 4.30 (s, 2H), 4.22 (d, 1H), 4.08 (dd, 1H), 3.93 (dd, 1H), 3.81-3.74 |
| | | | (m, 4H), 3.44-3.33 (m, 2H), 3.26 (d, 1H), 3.11 (t, 1H), 3.08 - 3.03 (m, |
| | | | 4H), 2.85 (dd, 1H), 2.16-2.08 (m, 1H), 1.88 (t, 1H) |
| 1062 | 467.52 | 468.3 | (400 MHZ, CD₃OD): δ 7.33-7.31 (m, 4H), 6.95-6.88 (m, 2H), 6.71 (s, |
| | | (M + H) | 1H), 6.67 (s, 1H), 4.31 (s, 2H), 4.26-4.17 (m, 3H), 4.13-4.05 (m, 3H), |
| | | | 3.93 (dd, 1H), 3.45-3.34 (m, 2H), 3.26 (d, 1H), 3.12 (t, 1H), 2.86 (dd, |
| | | | 1H), 2.17-2.07 (m, 1H), 1.88 (t, 1H) |
| 1063 | 448.52 | 449.4 | (400 MHZ, CD₃OD): δ 7.46-7.32 (m, 4H), 7.11-7.05 (m, 2H), 6.80 (dd, |
| | | (M + H) | 1H), 6.63 (dd, 1H), 6.43 (dd, 1H), 6.14 (dd, 1H), 4.49 - 4.27 (m, 3H), |
| | | | 4.13-4.08 (m, 1H), 4.00 (d, 1H), 3.56-3.37 (m, 3H), 3.17 (brt, 1H), 3.00- |
| | | | 2.85 (m, 1H), 2.44-2.02 (m, 2H) |
| 1064 | 461.52 | 462.2 | (400 MHZ, CD₃OD): δ 9.16 (dd, 1H), 8.09 (dd, 1H), 7.78 (dd, 1H), 7.64- |
| | | (M + H) | 7.52 (m, 2H), 7.41-7.33 (m, 4H), 7.00 (s, 1H), 4.43 (s, 2H), 4.28 (d, 1H), |
| | | | 4.09 (dd, 1H), 3.99-3.90 (m, 1H), 3.45-3.33 (m, 3H), 3.13 (t, 1H), 2.88 |
| | | | (dd, 1H), 2.29-1.85 (m, 2H) |
| 1065 | 461.52 | 462.3 | (400 MHZ, CD₃OD): δ 8.83 (d, 2H), 7.98 (s, 1H), 7.90 (s, 1H), 7.43-7.28 |
| | | (M + H) | (m, 5H), 6.96 (s, 1H), 4.41 (s, 2H), 4.23 (br d, 1H), 4.08 (dd, 1H), 3.92 |
| | | | (dd, 1H), 3.45-3.34 (m, 2H), 3.28 - 3.24 (m, 1H), 3.11 (t, 1H), 2.86 (dd, |
| | | | 1H), 2.13 (d, 1H), 1.88 (t, J = 10.9 Hz, 1H) |
| 1066 | 442.51 | 443.4 | (400 MHZ, CD₃OD): δ 7.36 (s, 1H), 7.30-7.18 (m, 3H), 7.12 (d, 2H), |
| | | (M + H) | 6.56 (d, 2H), 6.43 (s, 1H), 4.30 (s, 2H), 4.25-4.15 (m, 3H), 4.10-4.01 (m, |
| | | | 3H), 3.91 (dd, 1H), 3.42-3.33 (m, 2H), 3.25 (d, 1H), 3.12 (t, 1H), 2.85 |
| | | | (dd, 1H), 2.17-2.04 (m, 1H), 1.88 (t, 1H) |
| 1067 | 488.54 | 489.4 | (400 MHZ, CD₃OD): δ 8.50 (br s, 1H), 7.77 (d, 1H), 7.60-7.43 (m, 4H), |
| | | (M + H) | 7.08 (d, 1H), 4.83-4.74 (m, 5H), 4.27-4.11 (m, 3H), 4.10-4.05 (m, 3H), |
| | | | 3.68-3.54 (m, 6H), 3.10-3.00 (m, 2H), 2.77 (t, 1H) |
| 1068 | 443.54 | 444.2 | (400 MHZ, CD₃OD): δ 7.54-7.52 (m, 4H), 7.40 (d, 2H), 6.97 (d, 2H), |
| | | (M + H) | 4.84-4.82 (m, 1H), 4.51 (s, 2H), 4.33-4.11 (m, 3H), 4.06-3.99 (m, 4H), |
| | | | 3.67-3.55 (m, 2H), 3.55-3.51 (m, 4H), 3.40-3.32 (m, 1H), 3.18-3.05 (m, |
| | | | 2H), 2.84 (t, 1H) |
| 1069 | 488.54 | 489.4 | (400 MHZ, CD₃OD): δ 8.37 (d, 1H), 7.70 (dd, 1H), 7.55-7.53 (m, 4H), |
| | | (M + H) | 7.07 (d, 1H), 4.83-4.81 (m, 1H), 4.73 (s, 2H), 4.34-4.11 (m, 3H), 4.07- |
| | | | 4.00 (m, 4H), 3.71-3.48 (m, 6H), 3.37-3.33 (m, 1H), 3.21-3.05 (m, 2H), |
| | | | 2.84 (t, 1H) |
| 1075 | 534.45 | 534.3 | (400 MHZ, CD₃OD): δ 7.56 (d, 1H), 7.33 (d, 1H), 7.16-7.10 (m, 1H), |
| | | (M+) | 7.06-6.98 (m, 2H), 6.28-6.22 (m, 2H), 5.88 (t, 1H), 4.27 (t, 2H), 3.97- |
| | | | 3.84 (m, 2H), 3.58-3.50 (m, 4H), 3.47 (t, 2H), 3.40 (t, 1H), 3.24-3.14 (m, |
| | | | 2H), 3.11-3.02 (m, 1H), 2.96-2.82 (m, 3H), 2.42 (br t, 1H), 2.31 (br d, |
| | | | 1H) |
| 1076 | 425.53 | 426.3 | (400 MHZ, CD₃OD): δ 7.56 (d, 1H), 7.30 (d, 1H), 7.14-7.07 (m, 3H), |
| | | (M + H) | 7.05-7.00 (m, 2H), 6.65- 6.54 (m, 3H), 4.29 (t, 2H), 3.92 (dd, 1H), 3.86 |
| | | | (dd, 1H), 3.55-3.46 (m, 3H), 3.41-3.34 (m, 1H), 3.22-3.12 (m, 2H), 3.10- |
| | | | 3.04 (m, 1H), 2.96-2.86 (m, 3H), 2.38 (t, 1H), 2.28-2.23 (m, 1H) |
| 1077 | 514.58 | 515.4 | (400 MHZ, CD₃OD): δ 7.77 (d 1H), 7.22 (dd, 1H), 7.06 (d, 1H), 6.97- |
| | | (M + H) | 6.95 (m, 3H), 3.93 (dd, 1H), 3.89-3.82 (m, 1H), 3.62 (t, 1H), 3.55-3.46 |
| | | | (m, 1H), 3.39-3.34 (m, 1H), 3.21-3.15 (m, 1H), 3.15-3.07 (m, 1H), 3.06- |
| | | | 2.70 (m, 8H), 2.38 (br t, 1H), 2.28 (br d, 1H), 1.21 (d, 6H) |
| 1078 | 497.55 | 498.2 | (400 MHZ, CD₃OD): δ 8.64 (d, 1H), 7.98 (dd, 1H), 7.26-7.15 (m, 4H), |
| | | (M + H) | 7.14-7.07 (m, 3H), 3.92 (dd, 1H), 3.85 (dd, 1H), 3.72-3.63 (m, 2H), 3.55- |
| | | | 3.45 (m, 1H), 3.39-3.37 (m, 1H), 3.20-3.07 (m, 2H), 3.03-2.72 (m, 6H), |
| | | | 2.39 (t, 1H), 2.31-2.27 (m, 1H) |
| 1081 | 443.54 | 444.2 | (400 MHZ, CD₃OD): δ 7.59 (s, 1H), 7.57-7.49 (m, 3H), 7.46 (d, 2H), |
| | | (M + H) | 7.04 (d, 2H), 4.83 (d, 1H), 4.54 (s, 2H), 4.26-4.14 (m, 3H), 4.10-4.01 (m, |
| | | | 4H), 3.68-3.52 (m, 6H), 3.34 (s, 0.44H), 3.28 - 3.22 (m, 0.48H), 3.15- |
| | | | 3.02 (m, 2H), 2.80 (t, 1H) |
| 1082 | 423.51 | 424 | (400 MHZ, CD₃OD): δ 7.38 (s, 1H), 7.33-7.19 (m, 5H), 6.68 (dd, 1H), |
| | | (M + H) | 6.65-6.61 (m, 2H), 6.21 (dd, 1H), 6.07 (t, 1H), 4.32 (s, 2H), 4.23 (d, 1H), |
| | | | 4.07 (dd, 1H), 3.91 (dd, 1H), 3.44-3.34 (m, 2H), 3.26 (d, 1H), 3.12 (t, |
| | | | 1H), 2.86 (dd, 1H), 2.15-2.09 (m, 1H), 1.88 (t, 1H) |
| 1083 | 436.51 | 437.1 | (400 MHZ, CD₃OD): δ9.02 (dd, , 1H), 8.62 (dd, 1H), 8.14 (dd, 1H), 7.80 |
| | | (M + H) | (d, 2H), 7.46-7.26 (m, 4H), 6.78 (d, 2H), 4.66 (d, 1H), 4.42 (s, 2H), 4.09- |
| | | | 3.95 (m, 3H), 3.51-3.37 (m, 2H), 3.18-3.11 (m, 1H), 2.98-2.89 (m, 2H), |
| | | | 2.64 (t, 1H) |
| 1084 | 436.51 | 437.3 | (400 MHZ, CD₃OD): δ 9.02 (d, 2H), 8.22 (d, 2H), 7.63 (t, 1H), 7.58 (s, |
| | | (M + H) | 1H), 7.56-7.45 (m, 3H), 6.93 (d, 2H), 4.83 (d, 1H), 4.60 (s, 2H), 4.27- |
| | | | 4.11 (m, 3H), 3.71-3.53 (m, 2H), 3.36-3.33 (m, 1H), 3.17-3.06 (m, 2H), |
| | | | 2.82 (t, 1H) |
| 1086 | 488.54 | 489.2 | (400 MHZ, CD₃OD): δ 8.56 (t, J = 5.7 Hz, 1H), 8.14 (d, J = 2.0 Hz, 1H), |
| | | (M + H) | 7.43 (dd, J = 8.8, 2.1 Hz, 1H), 7.38 (s, 1H), 7.33-7.23 (m, 3H), 6.89 (d, |
| | | | J = 8.8 Hz, 1H), 4.62 (d, J = 4.4 Hz, 2H), 4.24 (d, J = 13.3 Hz, 1H), 4.05 |
| | | | (dd, J = 12.0, 2.6 Hz, 2H), 3.93-3.71 (m, 4H), 3.39-3.32 (m, 2H), 3.25 (d, |
| | | | J = 13.3 Hz, 1H), 3.10 (t, J = 9.0 Hz, 2H), 2.91-2.84 (m, 1H), 2.80 (dd, |
| | | | J = 11.2, 4.8 Hz, 1H), 2.43-2.18 (m, 2H), 2.13-2.07 (m, 1H), 1.85 (t, J = |
| | | | 10.8 Hz, 1H) |
| 1087 | 487.51 | 488.3 | (400 MHZ, DMSO-d₆): δ 8.72 (t, 1H), 8.01 (d, 1H), 7.54 (dd, 1H), 7.32 |
| | | (M + H) | (s, 1H), 7.30-7.18 (m, 3H), 6.89 (d, 1H), 6.85 (s, 1H), 4.77-4.68 (m, 2H), |
| | | | 4.64-4.57 (m, 3H), 4.36-4.18 (m, 4H), 4.08-4.04 (m, 2H), 3.97-3.89 (m, |
| | | | 1H), 3.66-3.58 (m, 1H), 3.18-3.03 (m, 3H), 2.98-2.87 (m, 1H), 2.61 (dd, |
| | | | 1H), 1.98 (d, 1H), 1.70 (t, 1H) |
| 1088 | 468.51 | 469.3 | (400 MHZ, DMSO-d₆): δ 11.29 (br s, 1H), 8.66 (t, 1H), 8.31 (d, 1H), |
| | | (M + H) | 7.75 (dd, 1H), 7.35 (s, 1H), 7.32-7.19 (m, 3H), 6.97 (d, 1H), 6.80-6.77 |
| | | | (m, 1H), 6.40 (br s, 1H), 6.10-6.03 (m, 1H), 4.79-4.69 (m, 2H), 4.65-4.57 |
| | | | (m, 3H), 4.33 (t, 1H), 4.25 ( d, 1H), 3.96-3.91 (m, 1H), 3.66-3.59 (m, |
| | | | 1H), 3.20-3.02 (m, 3H), 2.97-2.89 (m, 1H), 2.62 (dd, 1H), 1.99 (d, 1H), |
| | | | 1.71 (t, 1H) |
| 1089 | 481.51 | 482.3 | (400 MHZ, CD₃OD): δ 9.06 (d, 1H), 8.94 (d, 1H), 8.18 (dd, 1H), 8.13 |
| | | (M + H) | (dd, 1H), 7.73 (dd, 1H), 7.43 (s, 1H), 7.37-7.24 (m, 3H), 7.10 (d, 1H), |
| | | | 4.70 (s, 2H), 4.25 (d, 1H), 4.06 (dd, 1H), 3.90 (dd, 1H), 3.41-3.34 (m, |
| | | | 2H), 3.27 (d, 1H), 3.11 (t, 1H), 2.83 (dd, 1H), 2.13-2.09 (m, 1H), 1.87 (t, |
| | | | 1H) |
| 1090 | 481.51 | 482.3 | (400 MHZ, CD₃OD): δ 9.23 (d, 1H), 8.77 (d, 2H), 8.42 (dd, 1H), 7.43 (s, |
| | | (M + H) | 1H), 7.37-7.23 (m, 4H), 7.04 (d, 1H), 4.69 (s, 2H), 4.27 (d, 1H), 4.07 (dd, |
| | | | 1H), 3.91 (dd, 1H), 3.42-3.33 (m, 2H), 3.26 (d, 1H), 3.12 (t, 1H), 2.84 |
| | | | (dd, 1H), 2.15-2.09 (m, 1H), 1.88 (t, 1H) |
| 1092 | 443.54 | 444.3 | (400 MHZ, CD₃OD): δ 7.35-7.26 (m, 4H), 7.06 (d, 2H), 6.59 (d, 2H), |
| | | (M + H) | 4.29 (s, 2H), 4.22 (d, 1H), 4.08 (dd, 1H), 4.05-3.97 (m, 1H), 3.92 (dd, |
| | | | 1H), 3.88-3.78 (m, 2H), 3.66-3.53 (m, 1H), 3.42-3.33 (m, 2H), 3.24 (d, |
| | | | 1H), 3.12 (t, 1H), 3.04-2.93 (m, 1H), 2.91-2.79 (m, 2H), 2.37-2.16 (m, |
| | | | 2H), 2.15-2.09 (m, 1H), 1.87 (t, 1H) |
| 1093 | 442.51 | 443.3 | (400 MHZ, CD₃OD): δ 7.38-7.24 (m, 4H), 7.11 (d, 2H), 6.55 (d, 2H), |
| | | (M + H) | 6.42 (s, 1H), 4.29 (s, 2H), 4.25-4.15 (m, 3H), 4.08 (dd, 1H), 4.05-4.02 |
| | | | (m, 2H), 3.92 (dd, 1H), 3.42-3.35 (m, 2H), 3.25 (d, 1H), 3.11 (t, 1H), 2.85 |
| | | | (dd, 1H), 2.15-2.09 (m, 1H), 1.87 (t, 1H) |
| 1094 | 423.51 | 424.2 | (400 MHZ, CD₃OD): δ 7.40-7.25 (m, 6H), 6.69-6.60 (m, 3H), 6.21 (dd, |
| | | (M + H) | 1H), 6.07 (t, 1H), 4.31 (s, 2H), 4.22 (d, 1H), 4.09 (dd, 1H), 3.93 (dd, 1H), |
| | | | 3.43-3.33 (m, 2H), 3.25 (d, 1H), 3.12 (t, 1H), 2.86 (dd, 1H), 2.15-2.09 |
| | | | (m, 1H), 1.88 (t, 1H) |
| 1095 | 436.51 | 437.3 | (400 MHZ, DMSO-d₆): δ 9.01 (dd, 1H), 8.00 (dd, 1H), 7.90 (d, 2H), |
| | | (M + H) | 7.60 (dd, 1H), 7.37-7.21 (m, 4H), 6.78-6.65 (m, 3H), 4.76-4.69 (m, 2H), |
| | | | 4.59 (br s, 1H), 4.34-4.31 (m, 3H), 4.22 (d, 1H), 3.94 (d, 1H), 3.64 (dd, |
| | | | 1H), 3.17-3.02 (m, 3H), 2.92 (t, 1H), 2.62 (dd, 1H), 2.04-1.92 (m, 1H), |
| | | | 1.69 (t, 1H) |
| 1096 | 436.51 | 437.1 | (400 MHZ, DMSO-d₆): δ 8.72 (d, 2H), 8.12 (d, 2H), 7.35-7.24 (m, 4H), |
| | | (M + H) | 7.20 (t, 1H), 6.79 (t, 1H), 6.68 (d, 2H), 4.79-4.65 (m, 2H), 4.58 (d, 1H), |
| | | | 4.37-4.28 (m, 3H), 4.22 (d, 1H), 3.96-3.91 (m, 1H), 3.67-3.60 ( m, 1H), |
| | | | 3.19-3.02 (m, 3H), 2.97-2.85 (m, 1H), 2.61 (dd, 1H), 1.98 (d, 1H), 1.69 |
| | | | (t, 1H) |
| 1098 | 488.54 | 489.2 | (400 MHz, CD₃OD): δ 8.53 (t, 1H), 8.14 (d, 1H), 7.43 (dd, 1H), 7.36- |
| | | (M + H) | 7.34 (m, 4H), 6.91 (d, 1H), 4.65-4.56 (m, 2H), 4.24 (d, 1H), 4.08 (dd, |
| | | | 1H), 4.04-4.01 (m, 1H), 3.92 (dd, 1H), 3.89-3.70 (m, 3H), 3.43-3.33 (m, |
| | | | 2H), 3.25 (d, 1H), 3.12 (t, 2H), 2.84 (dd, 2H), 2.41-2.16 (m, 2H), 2.15- |
| | | | 2.10 (m, 1H), 1.88 (t, 1H) |
| 1099 | 487.51 | 488.3 | (400 MHZ, DMSO-d₆): δ 8.72 (t, 1H), 8.01 (d, 1H), 7.55 (dd, 1H), 7.38- |
| | | (M + H) | 7.22 (m, 4H), 6.91 (d, 1H), 6.86 (s, 1H), 4.77-4.56 (m, 5H), 4.32 (t, 1H), |
| | | | 4.26-4.15 (m, 3H), 4.10-4.02 (m, 2H), 3.99-3.87 (m, 1H), 3.68-3.56 (m, |
| | | | 1H), 3.17-3.02 (m,3H), 2.95-2.88 (m, 1H), 2.60 (dd, 1H), 1.98 (d, 1H), |
| | | | 1.69 (t, 1H) |
| 1100 | 468.51 | 469.2 | (400 MHZ, CD₃OD): δ 8.33 (d, 1H), 7.67 (dd, 1H), 7.42-7.30 (m, 4H), |
| | | (M + H) | 6.95 (d, 1H), 6.76 (dd, 1H), 6.36 (dd, 1H), 6.12 (dd, 1H), 4.62 (s, 2H), |
| | | | 4.25 (d, 1H), 4.09 (dd, 1H), 3.92 (dd, 1H), 3.42-3.34 (m, 2H), 3.26 (d, |
| | | | 1H), 3.12 (t, 1H), 2.85 (dd, 1H), 2.16-2.10 (m, 1H), 1.88 (t, 1H) |
| 1101 | 481.51 | 482.3 | (400 MHZ, DMSO-d₆): δ 9.14 (dd, 1H), 8.95-8.87 (m, 2H), 8.26 (dd, |
| | | (M + H) | 1H), 8.20 (dd, 1H), 7.72 (dd, 1H), 7.40-7.26 (m, 4H), 7.13 (d, 1H), 4.77- |
| | | | 4.67 (m, 4H), 4.58 (d, 1H), 4.33 (dd, 1H), 4.22 (d, 1H), 3.96-3.88 (m, |
| | | | 1H), 3.70-3.58 (m, 1H), 3.18-3.00 (m, 3H), 2.95-2.88 (m, 1H), 2.61 (dd, |
| | | | 1H), 1.98 (d, 1H), 1.70 (t, 1H) |
| 1102 | 481.51 | 482.3 | (400 MHz, DMSO-d₆): δ 9.14 (s, 1H), 8.92 (t, 1H), 8.84 (d, 2H), 8.40 |
| | | (M + H) | (d, 1H), 7.40-7.25 (m, 5H), 7.12 (d, 1H), 4.80-4.65 (m, 4H), 4.57 (d, 1H), |
| | | | 4.33 (t, 1H), 4.22 (d, 1H), 3.95-3.93 (m, 1H), 3.71-3.54 (m, 1H), 3.20- |
| | | | 3.01 (m, 3H), 2.95-2.88 (m, 1H), 2.60 (dd, 1H), 1.98 (d, 1H), 1.70 (t, 1H) |
| 1103 | 462.51 | 463.2 | (400 MHZ, DMSO-d₆): δ 8.08 (t, 1H), 7.71 (d, 1H), 7.37 (dd, 1H), 7.10 |
| | | (M + H) | (d, 1H), 4.70-4.56 (m, 3H), 4.10 (t, 1H), 3.73-3.49 (m, 2H), 3.35-3.32 (m, |
| | | | 2H), 3.24-3.12 (m, 1H), 3.07-2.99 (m, 1H), 2.92-2.85 (m, 1H), 2.77-2.64 |
| | | | (m, 2H), 2.45-2.36 (m, 1H), 1.96-1.83 (m, 2H), 1.77 (br t, 2H), 1.62-1.43 |
| | | | (m, 8H) |
| 1105 | 534.57 | 535.4 | (400 MHZ, CD₃OD): δ 7.81 (d, 1H), 7.30-7.22 (m, 4H), 7.21-7.12 (m, |
| | | (M + H) | 5H), 6.98 (d, 1H), 4.60-4.53 (m, 2H), 4.20 (d, 1H), 4.08 (dd, 1H), 3.96- |
| | | | 3.88 (m, 3H), 3.42-3.35 (m, 2H), 3.22 (d, 1H), 3.11 (t, 1H), 2.84 (dd, 1H), |
| | | | 2.14-2.08 (m, 1H), 1.86 (t, 1H) |
| 1106 | 478.55 | 479.1 | (400 MHZ, CD₃OD): δ 7.80 (d, 1H), 7.29-7.24 (m, 1H), 7.12-7.06 (m, |
| | | (M + H) | 1H), 3.94-3.76 (m, 2H), 3.52-3.43 (m, 1H), 3.42-3.33 (m, 3H), 3.18-3.09 |
| | | | (m, 1H), 2.99 (dd, 1H), 2.89-2.79 (m, 1H), 2.70-2.58 (m, 1H), 2.28-2.07 |
| | | | (m, 2H), 1.81-1.34 (m, 14H) |
| 1107 | 478.55 | 479.4 | (400 MHZ, CD₃OD): δ 7.80 (d, 1H), 7.27 (dd, 1H), 7.08 (d, 1H), 3.89- |
| | | (M + H) | 3.80 (m, 2H), 3.50-3.36 (m, 4H), 3.15-3.08 (m, 1H), 2.96 (dd, 1H), 2.90- |
| | | | 2.77 (m, 1H), 2.70-2.55 (m, 1H), 2.24-2.03 (m, 2H), 1.90-1.75 (m, 4H), |
| | | | 1.66-1.58 (m, 2H), 1.47-1.33 (m, 3H), 1.26-1.16 (m, 1H), 1.11-0.93 (m, |
| | | | 4H) |
| 1108 | 516.6 | 517.5 | (400 MHZ, CD₃OD): δ 8.09 (d, 1H), 7.50 (dd, 1H), 7.27-7.08 (m, 4H), |
| | | (M + H) | 6.98 (d, 1H), 4.03 (br s, 1H), 3.95-3.74 (m, 5H), 3.59 (t, 2H), 3.54-3.44 |
| | | | (m, 1H), 3.38-3.33 (m, 1H), 3.20-3.04 (m, 3H), 3.01-2.84 (m, 5H), 2.81- |
| | | | 2.69 (m, 2H), 2.35 (t, 2H), 2.27-2.22 (m, 2H) |
| 1109 | 509.56 | 510.4 | (400 MHZ, DMSO-d₆): δ 9.15 (dd, 1H), 8.90 (d, 1H), 8.40-8.32 (m, 2H), |
| | | (M + H) | 8.24 (dd, 1H), 7.74 (dd, 1H), 7.31 (d, 1H), 7.23 (d, 2H), 7.16 (d, 2H), |
| | | | 4.71-4.68 (m, 3H), 4.20 (br s, 1H), 3.80 (d, 1H), 3.71-3.64 (m, 2H), 3.61- |
| | | | 3.49 (m, 1H), 3.27-3.17 (m, 1H), 3.07-2.71 (m, 8H), 2.66-2.57 (m, 1H), |
| | | | 2.17 (t, 1H), 2.08 (br d, 1H) |
| 1110 | 508.58 | 509.4 | (400 MHZ, DMSO-d₆): δ 8.63-8.59 (m, 2H), 8.50 (d, 1H), 8.33 (t, 1H), |
| | | (M + H) | 8.05 (dd, 1H), 7.75-7.71 (m, 2H), 7.31-7.21 (m, 3H), 7.21-7.14 (m, 2H), |
| | | | 4.73-4.68 (m, 3H), 4.21 (dd, 1H), 3.84-3.79 (m, 1H), 3.71-3.64 (m, 2H), |
| | | | 3.62-3.50 (m, 1H), 3.27-3.21 (m, 1H), 3.08-3.00 (m, 1H), 2.99-2.68 (m, |
| | | | 7H), 2.67-2.59 (m, 1H), 2.18 (t, 1H), 2.12-2.08 (m, 1H) |
| 1111 | 509.56 | 510.5 | (400 MHZ, DMSO-d₆): δ 9.12 (d, 1H), 8.86 (d, 2H), 8.48 (dd, 1H), 8.42 |
| | | (M + H) | (t, 1H), 7.38 (t, 1H), 7.27 (d, 1H), 7.22 (d, 2H), 7.16 (d, 2H), 4.73-4.64 |
| | | | (m, 3H), 4.19 (dd, 1H), 3.84-3.76 (m, 1H), 3.70-3.63 (m, 2H), 3.59-3.51 |
| | | | (m, 1H), 3.27-3.19 (m, 1H), 3.06-2.98 (m, 1H), 2.98-2.66 (m, 7H), 2.65- |
| | | | 2.57 (m, 1H), 2.16 (t, 1H), 2.10-2.04 (m, 1H) |
| 1112 | 498.54 | 499.4 | (400 MHZ, DMSO-d₆): δ 8.59 (d, 1H), 8.43 (t, 1H), 8.17 (s, 1H), 8.05 |
| | | (M + H) | (dd, 1H), 7.34 (s, 1H), 7.28 (d, 1H), 7.25-7.12 (m, 4H), 4.72-4.65 (m, |
| | | | 3H), 4.19 (dd, 1H), 3.86-3.74 (m, 1H), 3.68-3.61 (m, 2H), 3.60-3.51 (m, |
| | | | 1H), 3.27-3.18 (m, 1H), 3.07-2.66 (m, 8H), 2.65-2.55 (m, 1H), 2.16 (t, |
| | | | 1H), 2.08 (d, 1H) |
| 1113 | 488.97 | 489.3 | (400 MHZ, CD₃OD): δ 9.24 (s, 1H), 7.23 (s, 2H), 7.20-7.13 (m, 4H), |
| | | (M + H) | 6.73 (s, 1H), 3.92 (dd, 1H), 3.84 (dd, 1H), 3.53-3.45 (m, 1H), 3.39-3.34 |
| | | | (m, 3H), 3.15 (t, 1H), 3.08 (dd, 1H), 3.01-2.67 (m, 6H), 2.34 (t, 1H), |
| | | | 2.26-2.21 (m, 1H) |
| 1114 | 535.66 | 536.2 | (400 MHZ, CD₃OD): δ 8.82 (d, 2H), 7.55 (s, 1H), 7.46 (s, 1H), 7.34 (t, |
| | | (M + H) | 1H), 6.75 (t, 1H), 3.95-3.76 (m, 2H), 3.51-3.39 (m, 1H), 3.37-3.33 (m, |
| | | | 1H), 3.20-3.08 (m, 3H), 2.99 (dd, 1H), 2.87-2.74 (m, 1H), 2.67-2.52 (m, |
| | | | 2H), 2.25-2.04 (m, 2H), 1.72-1.63 (m, 2H), 1.58-1.28 (m, 6H), 1.14-1.04 |
| | | | (m, 2H), 1.02-0.91 (m, 2H) |
| 1115 | 554.65 | 555.5 | (400 MHZ, DMSO-d₆): δ 8.15 (br t, 1H), 7.70 (d, 1H), 7.34 (dd, 1H), |
| | | (M + H) | 7.18 (d, 1H), 6.97 -6.87 (m, 3H), 4.79-4.63 (m, 3H), 4.14 (dd, 1H), 3.83- |
| | | | 3.74 (m, 1H), 3.63-3.51 (m, 2H), 3.28-3.17 (m, 2H), 3.06-3.00 (m, 1H), |
| | | | 2.97-2.82 (m, 4H), 2.81-2.70 (m, 1H), 2.68-2.58 (m, 2H), 2.44-2.36 (m, |
| | | | 2H), 2.16 (t, 1H), 2.10-2.03 (m, 1H), 1.82-1.62 (m, 5H), 1.46-1.27 (m, |
| | | | 5H) |
| 1116 | 478.97 | 479.2 | (400 MHZ, CD₃OD): δ 7.24-7.05 (m, 6H), 6.76 (t, 1H), 3.92 (dd, 1H), |
| | | (M + H) | 3.87 (s, 3H), 3.84 (dd, 1H), 3.54-3.44 (m, 1H), 3.35-3.32 (m, 3H), 3.15 (t, |
| | | | 1H), 3.08 (dd, 1H), 3.00-2.82 (m, 4H), 2.80-2.69 (m, 2H), 2.34 (t, 1H), |
| | | | 2.26-2.21 (m, 1H). |
| 1117 | 464.94 | 464.1 | (400 MHZ, CD₃OD): δ 7.20-7.12 (m, 4H), 7.02 (t, 1H), 6.97 (dd, 1H), |
| | | (M+) | 6.72 (t, 1H), 3.92 (dd , 1H), 3.87-3.84 (dd, 1H ), 3.53-3.48 (m, 1H), 3.37- |
| | | | 3.32 (m, 3H), 3.15 (t, 1H), 3.07 (dd, 1H), 3.01-2.83 (m, 4H), 2.82-2.68 |
| | | | (m, 2H), 2.34 (t, 1H), 2.26-2.21 (m, 1H) |
| 1118 | 460.53 | 460.3 | (400 MHZ, DMSO-d₆): δ 8.89 (d, 2H), 8.05 (s, 1H), 7.93 (br s, 1H), 7.78 |
| | | (M+) | (s, 1H), 7.43 (t, 1H), 7.21 (br s, 1H), 7.15 (t, 1H), 5.95 (t, 1H), 4.70-4.59 |
| | | | (m, 3H), 4.10 (br s, 1H), 3.77-3.67 (m, 1H), 3.63-3.43 (m, 1H), 3.26-3.13 |
| | | | (m, 1H), 3.13-3.00 (m, 3H), 2.96-2.85 (m, 1H), 2.84-2.69 (m, 2H), 2.43- |
| | | | 2.34 (m, 1H), 1.98-1.86 (m, 1H), 1.63-1.56 (m, 2H), 1.49-1.36 (m, 4H), |
| | | | 1.33-1.18 (m, 2H) |
| 1120 | 538.62 | 539.5 | (400 MHZ, CD₃OD): δ 7.22-7.13 (m, 4H), 6.96-6.91 (m, 1H), 6.87 (dd, |
| | | (M + H) | 1H), 6.33 (t, 1H), 3.94-3.81 (m, 3H), 3.80 (s, 3H), 3.55-3.42 (m, 1H), |
| | | | 3.38-3.33 (m, 3H), 3.15 (t, 1H), 3.08 (dd, 1H), 3.01-2.82 (m, 4H), 2.82- |
| | | | 2.69 (m, 2H), 2.55-2.47 (m, 4H), 2.34 (t, 1H), 2.27-2.02 (m, 3H) |
| 1121 | 526.63 | 527.4 | (400 MHZ, CD₃OD): δ 7.23-7.08 (m, 4H), 6.92 (s, 1H), 6.86 (s, 1H), |
| | | (M + H) | 6.32 (t, 1H), 3.92 (dd, 1H), 3.84 (dd, 1H), 3.79 (s, 3H), 3.55-3.41 (m, |
| | | | 1H), 3.37-3.32 (m, 3H), 3.15 (t, 1H), 3.08 (dd, 1H), 3.00-2.83 (m, 6H), |
| | | | 2.82-2.69 (m, 2H), 2.34 (t, 1H), 2.27-2.19 (m, 1H), 1.95-1.84 (m, 2H), |
| | | | 1.06 (t, 3H). |
| 1122 | 543.06 | 543.4 | (400 MHZ, CD₃OD): δ 7.21-7.13 (m, 6H), 6.72 (t, 1H), 3.97-3.79 (m, |
| | | (M+) | 3H), 3.54-3.44 (m, 1H), 3.38-3.33 (m, 3H), 3.15 (t, 1H), 3.07 (dd, 1H), |
| | | | 3.00-2.83 (m, 4H), 2.82-2.68 (m, 2H), 2.56-2.46 (m, 4H), 2.34 (t, 1H), |
| | | | 2.27-2.15 (m, 2H), 2.14-2.02 (m, 1H). |
| 1123 | 531.05 | 531.1 | (400 MHZ, CD₃OD): δ 7.21-7.13 (m, 6H), 6.72 (t, 1H), 3.92 (dd, 1H ), |
| | | (M+) | 3.84 (dd, 1H), 3.53-3.44 (m, 1H), 3.38-3.33 (m, 3H), 3.15 (t, 1H), 3.07 |
| | | | (dd, 1H), 2.99-2.91 (m, 3H), 2.90-2.83 (m, 3H), 2.82-2.69 (m, 2H), 2.34 |
| | | | (t, 1H), 2.26-2.21 (m, 1H), 1.95-1.84 (m, 2H), 1.06 (t, 3H). |
| 1124 | 490.6 | 491.4 | (400 MHZ, CD₃OD): δ 8.82 (d, 2H), 7.72 (s, 1H), 7.64 (t, 1H), 7.34 (t, |
| | | (M + H) | 1H), 6.87 (t, 1H), 5.36 (s, 1H), 3.90-3.80 (m, 2H), 3.49-3.44 (m, 1H), |
| | | | 3.38-3.34 (m, 7H), 3.18 (t, 2H), 3.12 (t, 1H), 3.00 (dd, 1H), 2.88-2.76 (m, |
| | | | 1H), 2.66-2.54 (m, 1H), 2.26-2.04 (m, 2H), 1.72-1.63 (m, 2H), 1.59-1.44 |
| | | | (m, 4H), 1.43-1.31 (m, 2H) |
| 1125 | 473.57 | 474.4 | (400 MHZ, CD₃OD): δ 8.81 (d, 2H), 7.69 (s, 1H), 7.43 (s, 1H), 7.34 (t, |
| | | (M + H) | 1H), 7.20 (t, 1H), 3.90-3.79 (m, 2H), 3.55-3.41 (m, 1H), 3.37-3.33 (m, |
| | | | 1H), 3.20-3.08 (m, 3H), 2.98 (dd, 1H), 2.86-2.75 (m, 1H), 2.62-2.52 (m, |
| | | | 1H), 2.20-2.02 (m, 5H), 1.72-1.63 (m, 2H), 1.58-1.44 (m, 4H), 1.42-1.31 |
| | | | (m, 2H) |
| 1126 | 487.6 | 488.1 | (400 MHZ, CD₃OD): δ 8.83 (d, 2H), 7.75 (s, 1H), 7.64 (s, 1H), 7.36 (t, |
| | | (M + H) | 1H), 6.76 (dd, 1H), 3.89-3.80 (m, 2H), 3.51-3.42 (m, 1H), 3.36-3.33 (m, |
| | | | 1H), 3.19 (t, 2H), 3.15-3.09 (m, 4H), 3.06 (s, 3H), 2.98 (dd, 1H), 2.86- |
| | | | 2.75 (m, 1H), 2.63-2.53 (m, 1H), 2.22-2.06 (m, 2H), 1.74-1.64 (m, 2H), |
| | | | 1.60-1.45 (m, 4H), 1.43-1.32 (m, 2H) |
| 1128 | 494.61 | 495.31 | (400 MHz, CD₃OD): δ 8.87 (d, 2H), 8.13 (t, 1H), 7.95 (dd, 1H), 7.40 (t, |
| | | (M + H) | 1H), 7.23-7.21 (m, 1H), 3.91-3.75 (m, 2H), 3.52-3.41 (m, 1H), 3.37-3.33 |
| | | | (m, 1H), 3.22 (t, 2H), 3.17-3.09 (m, 4H), 2.99 (dd, 1H), 2.88-2.73 (m, |
| | | | 1H), 2.65-2.52 (m, 1H), 2.23-2.02 (m, 2H), 1.76-1.63 (m, 2H), 1.59-1.45 |
| | | | (m, 4H), 1.44-1.31 (m, 2H) |
| 1129 | 444.53 | 445.2 | (400 MHZ, CD₃OD): δ 9.97 (s, 1H), 8.86 (d, 2H), 8.16 (s, 1H), 7.98 (dd, |
| | | (M + H) | 1H), 7.39 (t, 1H), 7.23 (dd, 1H), 3.90-3.80 (m, 2H), 3.52-3.41 (m, 1H), |
| | | | 3.37-3.33 (m, 1H), 3.22 (t, 2H), 3.12 (t, 1H), 3.00 (dd, 1H), 2.90-2.76 |
| | | | (m, 1H), 2.66-2.51 (m, 1H), 2.26-2.04 (m, 2H), 1.75-1.76 (m, 2H), 1.61- |
| | | | 1.45 (m, 4H), 1.43-1.34 (m, 2H) |
| 1130 | 441.53 | 442.24 | (400 MHZ, CD₃OD): δ 8.85 (d, 2H), 7.94-7.91 (m, 1H), 7.89 (s, 1H), |
| | | (M + H) | 7.39 (t, 1H), 6.97 (dd, 1H), 3.90 -3.79 (m, 2H), 3.52-3.42 (m, 1H), 3.39- |
| | | | 3.33 (m, 1H), 3.21-3.14 (m, 2H), 3.13-3.09 (m, 1H), 3.00 (dd, 1H), 2.88- |
| | | | 2.76 (m, 1H), 2.65-2.51 (m, 1H), 2.23-2.06 (m, 2H), 1.73-1.64 (m, 2H), |
| | | | 1.60-1.44 (m, 4H), 1.42-1.32 (m, 2H) |
| 1131 | 484.52 | 485.3 | (400 MHZ, CD₃OD): δ 8.85 (d, 2H), 7.87 (s, 2H), 7.38 (t, 1H), 6.95 (s, |
| | | (M + H) | 1H), 3.94-3.78 (m, 2H), 3.50-3.42 (m, 1H), 3.38-3.33 (m, 2H), 3.20 (t, |
| | | | 1H), 3.12 (t, 1H), 2.99 (dd, 1H), 2.88-2.75 (m, 1H), 2.64-2.52 (m, 1H), |
| | | | 2.22-2.05 (m, 2H), 1.74-1.65 (m, 2H), 1.60-1.43 (m, 4H), 1.43-1.26 (m, |
| | | | 2H) |
| 1133 | 441.53 | 442.4 | (400 MHZ, CD₃OD): δ 7.20-7.11 (m, 4H), 6.47-6.42 (m, 2H), 6.38 (t, |
| | | (M + H) | 1H), 3.92 (dd, 1H), 3.84 (dd, 1H), 3.75 (s, 3H), 3.54-3.44 (m, 1H), 3.34- |
| | | | 3.28 (m, 3H), 3.15 (t, 1H), 3.07 (dd, 1H), 3.01-2.87 (m, 2H), 2.86-2.81 |
| | | | (m, 2H), 2.80-2.70 (m, 2H), 2.34 (t, 1H), 2.26-2.21 (m, 1H) |
| 1134 | 445.94 | 446.2 | (400 MHZ, CD₃OD): δ 7.20-7.10 (m, 4H), 6.83-6.79(m, 2H), 6.74 (dd, |
| | | (M + H) | 1H), 3.92 (dd, 1H ), 3.84 (dd, 1H), 3.55-3.43 (m, 1H), 3.37-3.32 (m, 3H), |
| | | | 3.15 (t, 1H), 3.07 (dd, 1H), 3.00-2.82 (m, 4H), 2.80-2.69 (m, 2H), 2.34 (t, |
| | | | 1H), 2.26-2.21(m, 1H) |
| 1136 | 423.51 | 424.41 | (400 MHZ, CD₃OD): δ7.84 (d, 1H), 7.27 (dd, 1H), 6.93 (d, 1H), 3.89- |
| | | (M + H) | 3.79 (m, 2H), 3.48-3.42 (m, 1H), 3.37-3.32 (m, 3H), 3.12 (t, 1H), 2.98 |
| | | | (dd, 1H), 2.88-2.75 (m, 1H), 2.62-2.51 (m, 1H), 2.25-2.05 (m, 2H), 1.91- |
| | | | 1.81 (m, 1H), 1.75-1.68 (m, 2H), 1.60-1.43 (m, 4H), 1.42-1.31 (m, 2H), |
| | | | 0.96-0.87 (m, 2H), 0.65-0.56 (m, 2H) |
| 1138 | 459.54 | 460.4 | (400 MHZ, , CD₃OD): δ 8.38 (d, J = 2.4 Hz, 1H), 8.15 (br t, J = 5.0 Hz, |
| | | (M + H) | 1H), 7.82 (dd, J =8.9, 2.3 Hz, 1H), 7.61-7.56 (m, 2H), 7.46-7.39 (m, 2H), |
| | | | 7.34-7.28 (m, 1H), 7.11 (d, J = 9.0 Hz, 1H), 3.90-3.80 (m, 2H), 3.54-3.38 |
| | | | (m, 2H), 3.37-3.33 (m, 1H), 3.12 (t, J = 9.0 Hz, 1H), 3.00 (dd, J = 11.2, |
| | | | 4.9 Hz, 1H), 2.87-2.77 (m, 1H), 2.65-2.52 (m, 1H), 2.24-2.09 (m, 2H), |
| | | | 1.81-1.72 (m, 2H), 1.62-1.47 (m, 4H), 1.45-1.33 (m, 2H) |
| 1140 | 551.4 | 551.3 | (400 MHZ, CD₃OD): δ 8.07 (br t, 1H), 7.79 (d, 1H), 7.32-7.22 (m, 3H), |
| | | (M+) | 7.14-7.08 (m, 2H), 3.94 (dd, 1H), 3.84 (dd, 1H), 3.68-3.59 (m, 2H), 3.54- |
| | | | 3.42 (m, 1H), 3.15 (t, 1H), 3.06 (dd, 1H), 3.00-2.92 (m, 3H), 2.91-2.70 |
| | | | (m, 4H), 2.33 (t, 1H), 2.27-2.21 (m, 1H) |
| 1141 | 452.59 | 453.4 | (400 MHZ, CD₃OD): δ 6.46-6.35 (m, 1H), 6.19-6.03 (m, 2H), 3.91-3.80 |
| | | (M + H) | (m, 3H), 3.79-3.71 (m, 4H), 3.70-3.65 (m, 1H), 3.59-3.54 (m, 1H), 3.50- |
| | | | 3.41 (m, 2H), 3.36-3.34 (m, 3H), 3.15-3.08 (m, 2H), 3.05 (t, 1H), 2.98 |
| | | | (dd, 1H), 2.88-2.75 (m, 1H), 2.64-2.45 (m, 2H), 2.21-2.06 (m, 2H), 2.06- |
| | | | 1.97 (m, 1H), 1.69-1.58 (m, 2H), 1.57-1.42 (m, 4H), 1.41-1.31 (m, 2H). |
| 1143 | 521.96 | 522.1 | (400 MHZ, CD₃OD): δ 8.23 (br 1H), 7.79 (d, 1H), 7.24 (dd, 1H), 7.13 |
| | | (M + H) | (d, 1H), 6.49-6.44 (m, 2H), 6.41 (s, 1H), 3.92 (dd, 1H), 3.84 (dd, 1H), |
| | | | 3.64-3.57 (m, 2H), 3.49-3.40 (m, 3H), 3.35-3.32 (m, 1H), 3.20-3.10 (m, |
| | | | 1H), 3.04 (dd, 1H), 2.98-2.80 (m, 2H), 2.72-2.57 (m, 2H), 2.32 (t, 1H), |
| | | | 2.26-2.18 (m, 1H) |
| 1145 | 458.6 | 459.4 | (400 MHZ, CD₃OD): δ 7.27-7.10 (m, 5H), 6.12-5.99 (m, 3H), 3.89-3.82 |
| | | (M + H) | (m, 2H), 3.80 (s, 2H), 3.68 (s, 3H), 3.51-3.42 (m, 1H), 3.37-3.34 (m, 1H), |
| | | | 3.12 (t, 1H), 3.04-2.94 (m, 3H), 2.87-2.71 (m, 1H), 2.63-2.49 (m, 1H), |
| | | | 2.22-2.04 (m, 2H), 1.63-1.24 (m, 8H) |
| 1146 | 438.57 | 439.3 | (400 MHZ, CD₃OD): δ 6.23 (s, 1H), 6.18 (s, 1H), 6.09 (t, 1H), 4.74 (t, |
| | | (M + H) | 1H), 4.09-4.01 (m, 1H), 3.92-3.87 (m, 1H), 3.87-3.80 (m, 2H), 3.73 (s, |
| | | | 3H), 3.51-3.42 (m, 1H), 3.38-3.35 (m, 1H), 3.15-3.03 (m, 3H), 2.98 (dd, |
| | | | 1H), 2.88-2.72 (m, 1H), 2.64-2.50 (m, 1H), 2.37-2.22 (m, 1H), 2.20 -2.07 |
| | | | (m, 2H), 2.06-1.94 (m, 2H), 1.81-1.74 (m, 1H), 1.66-1.57 (m, 2H), 1.56- |
| | | | 1.40 (m, 4H), 1.40-1.26 (m, 2H) |
| 1147 | 436.55 | 437.3 | (400 MHZ, CD₃OD): δ 6.20 (s, 1H), 6.16-6.09 (m, 2H), 6.07-6.02 (m, |
| | | (M + H) | 1H), 5.92-5.86 (m, 1H), 5.63-5.59 (m, 1H), 4.81-4.77 (m, 1H), 4.74-4.65 |
| | | | (m, 1H), 3.87-3.83 (m, 2H), 3.72 (s, 3H), 3.51-3.43 (m, 1H), 3.38-3.33 |
| | | | (m, 1H), 3.12 (t, 1H), 3.05 (t, 2H), 2.99 (dd, 1H), 2.86-2.75 (m, 1H), |
| | | | 2.63-2.53 (m, 1H), 2.24-2.04 (m, 2H), 1.66-1.56 (m, 2H), 1.55-1.40 (m, |
| | | | 4H), 1.39-1.30 (m, 2H) |
| 1148 | 450.62 | 451.5 | (400 MHZ, CD₃OD): δ 6.17-5.97 (m, 3H), 3.93-3.77 (m, 2H), 3.72 (s, |
| | | (M + H) | 3H), 3.51-3.41 (m, 1H), 3.39-3.33 (m, 1H), 3.12 (t, 1H), 3.05 (t, 2H), |
| | | | 2.99 (dd, 1H), 2.90-2.71 (m, 1H), 2.66-2.48 (m, 1H), 2.37-2.32 (m, 1H), |
| | | | 2.20-2.07 (m, 2H), 1.92-1.68 (m, 5H), 1.66-1.57 (m, 2H), 1.56-1.24 (m, |
| | | | 11H) |
| 1149 | 448.26 | 449.3 | (400 MHZ, CD₃OD): δ 6.27 (s, 1H), 6.24-6.21 (m, 1H), 6.09 (t, 1H), |
| | | (M + H) | 6.05-6.00 (m, 1H), 3.89-3.80 (m, 2H), 3.73 (s, 3H), 3.51-3.43 (m, 1H), |
| | | | 3.38 -3.33 (m, 1H), 3.12 (t, 1H), 3.06 (t, 2H), 2.99 (dd, 1H), 2.87-2.74 |
| | | | (m, 1H), 2.64-2.52 (m, 1H), 2.40-2.30 (m, 2H), 2.23-2.07 (m, 4H), 1.81- |
| | | | 1.73 (m, 2H), 1.70-1.58 (m, 4H), 1.57-1.40 (m, 4H), 1.39-1.29 (m, 2H) |
| 1150 | 444.57 | 445.54 | (400 MHZ, CD₃OD): δ 7.57-7.52 (m, 2H), 7.42-7.35 (m, 2H), 7.31-7.26 |
| | | (M + H) | (m, 1H), 6.47 (s, 1H), 6.43-6.41 (m, 1H), 6.20 (t, 1H), 3.89-3.83 (m, 2H), |
| | | | 3.79 (s, 3H), 3.50-3.42 (m, 1H), 3.37-3.33 (m, 1H), 3.16-3.09 (m, 3H), |
| | | | 2.99 (dd, 1H), 2.86-2.75 (m, 1H), 2.64-2.52 (m, 1H), 2.20-2.07 (m, 2H), |
| | | | 1.72-1.59 (m, 2H), 1.58-1.43 (m, 4H), 1.41-1.27 (m, 2H) |
| 1151 | 472.63 | 473.3 | (400 MHZ, CD₃OD): δ 8.82 (d, 2H), 7.75 (s, 1H), 7.48 (dd, 1H), 7.34 (t, |
| | | (M + H) | 1H), 6.88 (t, 1H), 3.93-3.75 (m, 2H), 3.50-3.42 (m, 1H), 3.38-3.33 (m, |
| | | | 1H), 3.18 (t, 2H), 3.12 (t, 1H), 2.99 (dd, 1H), 2.90-2.73 (m, 1H), 2.66- |
| | | | 2.48 (m, 1H), 2.24-2.05 (m, 2H), 1.73-1.64 (m, 2H), 1.61-1.44 (m, 4H), |
| | | | 1.43-1.27 (m, 12H) |
| 1153 | 482.58 | 483.4 | (400 MHZ, CD₃OD): δ 7.49 (s, 1H), 7.19-7.14 (m, 4H), 6.64 (dd, 1H), |
| | | (M + H) | 6.60-6.57 (m, 1H), 6.55 (dd, 1H), 6.47 (dd, 1H), 6.13 (t, 1H), 3.92 (dd, |
| | | | 1H), 3.84 (dd, 1H), 3.77 (s, 3H), 3.53-3.44 (m, 1H), 3.37-3.33 (m, 3H), |
| | | | 3.15 (t, 1H), 3.08 (dd, 1H), 3.01-2.83 (m, 4H), 2.83-2.70 (m, 2H), 2.34 (t, |
| | | | 1H), 2.27-2.21 (m, 1H) |
| 1154 | 498.62 | 499.4 | (400 MHZ, CD₃OD): δ 7.22-7.09 (m, 4H), 6.30-6.26 (m, 2H), 6.12 (s, |
| | | (M + H) | 1H), 6.09-6.06 (m, 1H), 4.28-4.25 (m, 2H), 3.95-3.81 (m, 4H), 3.74 (s, |
| | | | 3H), 3.53-3.44 (m, 1H), 3.38-3.33 (m, 1H), 3.29-3.27 (m, 2H), 3.15 (t, |
| | | | 1H), 3.08 (dd, 1H), 3.02-2.66 (m, 6H), 2.46-2.44 (m, 2H), 2.35 (t, 1H), |
| | | | 2.26-2.21 (m, 1H) |
| 1155 | 500.59 | 501.4 | (400 MHZ, CD₃OD): δ 7.17-7.14 (m, 4H), 6.62 (s, 1H), 6.32-6.30 (m, |
| | | (M + H) | 1H), 6.27 (dd, 1H), 6.08 (t, 1H), 4.21-4.18 (m, 2H), 4.10-4.06 (m, 2H), |
| | | | 3.92 (dd, 1H), 3.84 (dd, 1H), 3.72 (s, 3H), 3.53-3.45 (m, 1H), 3.37-3.32 |
| | | | (m, 2H), 3.15 (t, 1H), 3.08 (dd, 1H), 3.03-2.66 (m, 7H), 2.34 (t, , 1H), |
| | | | 2.26-2.21 (m, 1H) |
| 1156 | 494.59 | 495.4 | (400 MHZ, CD₃OD): δ 8.81 (d, 2H), 7.36-7.31 (m, 2H), 7.25 (dd, 1H), |
| | | (M + H) | 7.21-7.14 (m, 4H), 6.35 (t, 1H), 3.95-3.84 (m, 2H), 3.82 (s, 3H), 3.54- |
| | | | 3.43 (m, 1H), 3.41-3.34 (m, 3H), 3.15 (t, 1H), 3.08 (dd, 1H), 3.01-2.84 |
| | | | (m, 4H), 2.83-2.69 (m, 2H), 2.35 (t, 1H), 2.27-2.21 (m, 1H) |
| 1157 | 494.59 | 495.4 | (400 MHZ, CD₃OD): δ 9.10 (dd, 1H), 8.06 (dd, 1H), 7.74 (dd, 1H), |
| | | (M + H) | 7.22-7.11 (m, 4H), 6.92-6.85 (m, 2H), 6.34 (t, 1H), 3.94-3.84 (m, 2H), |
| | | | 3.83 (s, 3H), 3.53-3.44 (m, 1H), 3.42-3.33 (m, 3H), 3.15 (t, 1H), 3.07 (dd, |
| | | | 1H), 3.00-2.83 (m, 4H), 2.82-2.69 (m, 2H), 2.34 (t, 1H), 2.26-2.20 (m, |
| | | | 1H) |
| 1159 | 486.99 | 487.55 | (400 MHZ, CD₃OD): δ 7.52 (s, 1H), 7.20-7.14 (m, 4H), 6.89 (s, 1H), |
| | | (M + H) | 6.82 (s, 1H), 6.70 (d, 1H), 6.50-6.47 (m, 2H), 3.92 (dd, 1H), 3.84 (dd, |
| | | | 1H), 3.53-3.45 (m, 1H), 3.38-3.33 (m, 3H), 3.15 (t, 1H), 3.08 (dd, 1H), |
| | | | 2.99-2.83 (m, 4H), 2.82-2.69 (m, 2H), 2.34 (t, 1H), 2.26-2.21 (m, 1H) |
| 1160 | 503.04 | 503.03 | (400 MHZ, CD₃OD): δ 7.16-7.14 (m, 4H), 6.63 (s, 1H), 6.53 (s, 1H), |
| | | (M+) | 6.48 (s, 1H), 6.12-6.09 (m, 1H), 4.28-4.24 (m, 2H), 3.94-3.81 (m, 4H), |
| | | | 3.53-3.44 (m, 1H), 3.37-3.25 (m, 3H), 3.15 (t, 1H), 3.08 (dd, 1H), 3.01- |
| | | | 2.81 (m, 4H), 2.80-2.69 (m, 2H), 2.47-2.40 (m, 2H), 2.34 (t, 1H), 2.26- |
| | | | 2.21 (m, 1H) |
| 1161 | 505.01 | 505.01 | (400 MHZ, CD₃OD): δ 7.16-7.14 (m, 4H), 6.65 (s, 1H), 6.61 (s, 1H), |
| | | (M+) | 6.55 (s, 1H), 6.43 (s, 1H), 4.23-4.17 (m, 2H), 4.11-4.07 (m, 2H), 3.92 |
| | | | (dd, 1H), 3.84 (dd, 1H), 3.54-3.43 (m, 1H), 3.37-3.33 (m, 1H), 3.29-3.26 |
| | | | (m, 2H), 3.15 (t, 1H), 3.08 (dd, 1H), 3.01-2.81 (m, 4H), 2.80-2.70 (m, |
| | | | 2H), 2.34 (t, 1H), 2.26-2.21 (m, 1H) |
| 1162 | 499.01 | 499.4 | (400 MHZ, CD₃OD): δ 8.82 (d, 2H), 7.59-7.57 (m, 2H), 7.35 (t, 1H), |
| | | (M+) | 7.21-7.13 (m, 4H), 6.72 (t, 1H), 3.92 (dd, 1H), 3.84 (dd, 1H), 3.54-3.44 |
| | | | (m, 1H), 3.41-3.34 (m, 3H), 3.15 (t, 1H), 3.07 (dd, 1H), 3.00-2.84 (m, |
| | | | 4H), 2.82-2.70 (m, 2H), 2.34 (t, 1H), 2.26-2.21 (m, 1H) |
| 1163 | 499.01 | 499.02 | (400 MHZ, CD₃OD): δ 9.13 (dd, 1H), 8.07 (dd, 1H), 7.76 (dd, 1H), |
| | | (M+) | 7.25-7.11 (m, 6H), 6.73 (t, 1H), 3.91 (dd, 1H), 3.84 (dd, 1H), 3.54-3.44 |
| | | | (m, 1H), 3.43-3.33 (m, 3H), 3.15 (t, 1H), 3.07 (dd, 1H), 3.00-2.83 (m, |
| | | | 4H), 2.81-2.67 (m, 2H), 2.34 (t, 1H), 2.26-2.20 (m, 1H) |
| 1166 | 520.55 | 521.4 | (400 MHZ, DMSO-d₆): δ 8.75 (t, 1H), 7.74 (d, 1H), 7.63 (d, 2H), 7.58 |
| | | (M + H) | (d, 2H), 7.43 (d, 2H), 7.38 (d, 2H), 7.31 (dd, 1H), 7.03 (d, 1H), 4.76 (d, |
| | | | 1H), 4.73-4.66 (m, 3H), 4.60 (d, 1H), 4.37 (dd, 1H), 4.27 (d, 1H), 3.99- |
| | | | 3.93(m, 1H), 3.70-3.60 (m, 1H), 3.21-3.03 (m, 3H), 2.99-2.88 (m, 1H), |
| | | | 2.72-2.58 (m, 1H), 2.06-1.98 (m, 1H), 1.74 (t, 1H) |
| 1169 | 444.45 | 445.3 | (400 MHZ, CD₃OD): δ 7.82 (d, 1H), 7.38 (s, 1H), 7.34-7.24 (m, 3H), |
| | | (M + H) | 7.18 (dd, 1H), 6.99 (d, 1H), 4.62 (s, 2H), 4.24 (d, 1H), 4.06 (dd, 1H), 3.90 |
| | | | (dd, 1H), 3.41-3.33 (m, 2H), 3.27 (d, 1H), 3.11 (t, 1H), 2.81 (dd, 1H), |
| | | | 2.13-2.07 (m, 1H), 1.86 (t, 1H) |
| 1170 | 444.45 | 445.3 | (500 MHZ, CD₃OD): δ 7.83 (d, 1H), 7.38-7.32 (m, 4H), 7.18 (dd, 1H), |
| | | (M + H) | 7.01 (d, 1H), 4.61 (s, 2H), 4.25 (d, 1H), 4.09 (dd, 1H), 3.93 (dd, 1H), |
| | | | 3.43-3.34 (m, 2H), 3.26 (d, 1H), 3.13 (t, 1H), 2.85 (dd, 1H), 2.15-2.11 |
| | | | (m, 1H), 1.88 (t, 1H) |
| 1171 | 554.65 | 555.5 | (400 MHZ, CD₃OD (0.6 mL) + CDCl₃ (0.2 mL)): δ 7.79 (d, 1H), 7.23- |
| | | (M + H) | 7.15 (m, 2H), 7.08-7.00 (m, 3H), 3.92 (br s, 2H), 3.63-3.50 (m, 3H), 3.41 |
| | | | (brt, 1H), 3.25-3.12 (m, 2H), 3.11-2.87 (m, 4H), 2.82-2.65 (m, 3H), 2.55- |
| | | | 2.28 (m, 2H), 1.84-1.81 (m, 2H), 1.77-1.65 (m, 3H), 1.52-1.21 (m, 5H) |
| 1173 | 530.54 | 531.3 | (400 MHZ, CD₃OD): δ 7.78 (d, 1H), 7.24 (dd, 1H), 7.11 (d, 1H), 6.73- |
| | | (M + H) | 6.63 (m, 2H), 4.36-4.26 (m, 4H), 3.99-3.85 (m, 2H), 3.62-3.45 (m, 3H), |
| | | | 3.42-3.35 (m, 1H), 3.15 (t, 1H), 3.06 (dd, 1H), 2.96 (t, 2H), 2.93-2.86 (m, |
| | | | 2H), 2.77-2.69 (m, 2H), 2.41 (t, 1H), 2.27-2.23 (m, 1H) |
| 1174 | 464.5 | 465.3 | (400 MHZ, CD₃OD): δ 7.82 (d, 1H), 7.23 (dd, 1H), 7.12 (d, 1H), 6.98 |
| | | (M + H) | (d, 2H), 4.74 (s, 2H), 3.92 (dd, 1H), 3.85 (dd, 1H), 3.52-3.41 (m, 1H), |
| | | | 3.30-3.28 (m, 1H), 3.14 (t, 1H), 3.07-2.97 (m, 2H), 2.92-2.82 (m, 1H), |
| | | | 2.81-2.70 (m, 2H), 2.29 (t, 1H), 2.24-2.18 (m, 1H) |
| 1177 | 511.54 | 512.2 | (400 MHZ, DMSO-d₆): δ 8.18 (t, 1H), 7.64 (d, 1H), 7.50 (d, 1H), 7.44 |
| | | (M + H) | (d, 1H), 7.21 (dd, 1H), 7.17 (s, 1H), 7.10-6.95 (m, 3H), 4.77-4.64 (m, |
| | | | 3H), 4.37 (t, 2H), 4.16 (dd, 1H), 3.86-3.69 (m, 3H), 3.68-3.56 (m, 1H), |
| | | | 3.30-3.21 (m, 1H), 3.14-3.04 (m, 1H), 3.00-2.89 (m, 3H), 2.86-2.69 (m, |
| | | | 3H), 2.20 (t, 1H), 2.14-2.06 (m, 1H) |
| 1178 | 522.56 | 523.4 | (400 MHZ, DMSO-d₆): δ 8.27 (t, 1H), 8.24-8.18 (m, 1H), 8.18-8.10 (m, |
| | | (M + H) | 1H), 7.70 (d, 1H), 7.59-7.52 (m, 2H), 7.39 (d, 1H), 7.36-7.30 (m, 2H), |
| | | | 7.17 (d, 1H), 4.76-4.70 (m, 3H), 4.27 (dd, 1H), 3.87-3.82 (m, 1H), 3.74- |
| | | | 3.67 (m, 2H), 3.64-3.54 (m, 1H), 3.43-3.33 (m, 2H), 3.29-3.22 (m, 1H), |
| | | | 3.22-3.11 (m, 2H), 3.10-2.84 (m, 5H), 2.37-2.29 (m, 1H), 2.20-2.15 (m, |
| | | | 1H) |
| 1179 | 478.52 | 479.2 | (400 MHZ, CD₃OD): δ 7.79 (d, 1H), 7.30-7.21 (m, 1H), 7.08 (d, 1H), |
| | | (M + H) | 6.91 (s, 1H), 6.83 (s, 1H), 3.92 (dd, 1H), 3.85 (dd, 1H), 3.70-3.58 (m, |
| | | | 2H), 3.54-3.42 (m, 1H), 3.39-3.34 (m, 1H), 3.20-3.11 (m, 3H), 3.09-2.96 |
| | | | (m, 2H), 2.93-2.69 (m, 3H), 2.31 (t, 1H), 2.26 -2.18 (m, 1H) |
| 1182 | 453.58 | 454.3 | (400 MHZ, CD₃OD): δ 6.28-6.20 (m, 2H), 6.11 (t, 1H), 4.03 (br s, 1H), |
| | | (M + H) | 3.92-3.79 (m, 4H), 3.73 (s, 3H), 3.70-3.56 (m, 1H), 3.51-3.43 (m, 1H), |
| | | | 3.38-3.33 (m, 1H), 3.16-2.95 (m, 5H), 2.86-2.74 (m, 2H), 2.63-2.50 (m, |
| | | | 1H), 2.41-2.05 (m, 4H), 1.66-1.57 (m, 2H), 1.55-1.40 (m, 4H), 1.39-1.27 |
| | | | (m, 2H) |
| 1183 | 458 | 458.4 | (400 MHZ, CD₃OD): δ 6.57 (s, 1H), 6.52 (s, 1H), 6.49 (s, 1H), 4.11-3.94 |
| | | (M+) | (m, 1H), 3.89-3.80 (m, 4H), 3.71-3.67 (m, 1H), 3.51-3.42 (m, 1H), 3.37- |
| | | | 3.33 (m, 1H), 3.12 (t, 2H), 3.06 (t, 2H), 2.98 (dd, 1H), 2.86-2.76 (m, 2H), |
| | | | 2.62-2.51 (m, 1H), 2.39-2.21 (m, 2H), 2.20-2.06 (m, 2H), 1.67-1.57 (m, |
| | | | 2H), 1.55-1.41 (m, 4H), 1.39-1.31 (m, 2H) |
| 1184 | 444.53 | 445.4 | (400 MHZ, CD₃OD): δ 9.91 (s, 1H), 8.76 (d, 2H), 8.70-8.64 (m, 1H), |
| | | (M + H) | 8.63 (d, 1H), 8.46 (dd, 1H), 7.24 (t, 1H), 6.92 (d, 1H), 3.90-3.81 (m, 2H), |
| | | | 3.51-3.43 (m, 1H), 3.41-3.34 (m, 3H), 3.13 (t, 1H), 3.01 (dd, 1H), 2.90- |
| | | | 2.78 (m, 1H), 2.66-2.54 (m, 1H), 2.26-2.09 (m, 2H), 1.81-1.66 (m, 2H), |
| | | | 1.61-1.46 (m, 4H), 1.44-1.34 (m, 2H) |
| 1186 | 478.52 | 479.1 | (400 MHZ, CD₃OD): δ 7.79 (d, 1H), 7.25 (dd, 1H), 7.08 (d, 1H), 6.73 (d, |
| | | (M + H) | 1H), 6.68 (d, 1H), 3.92- 3.88 (m, 1H), 3.85-3.80 (m, 1H), 3.62 (t, 2H), |
| | | | 3.53-3.41 (m, 1H), 3.36-3.32 (m, 1H), 3.18-3.11 (m, 3H), 3.08-3.00 (m, |
| | | | 2H), 2.99-2.85 (m, 3H), 2.30 (t, 1H), 2.26-2.20 (m, 1H) |
| 1187 | 474.48 | 475.2 | (400 MHZ, CD₃OD): δ 8.38 (d, 2H), 7.78 (d, 1H), 7.26 (dd, 1H), 7.14 (d, |
| | | (M + H) | 1H), 3.97 (dd, 1H), 3.87-3.76 (m, 3H), 3.48-3.39 (m, 1H), 3.35- 3.28 (m, |
| | | | 1H), 3.28-3.11 (m, 4H), 3.10-2.96 (m, 4H), 2.35-2.20 (m, 2H) |
| 1188 | 474.48 | 475.3 | (400 MHZ, CD₃OD): δ 8.58 (d, 1H), 7.78 (d, 1H), 7.31-7.24 (m, 2H), |
| | | (M + H) | 7.17 (d, 1H), 3.97 (dd, 1H), 3.91-3.80 (m, 3H), 3.47-3.39 (m, 1H), 3.36- |
| | | | 3.33 (m, 1H), 3.30-3.24 (m, 3H), 3.14 (t, 1H), 3.08-2.96 (m, 4H), 2.32- |
| | | | 2.19 (m, 2H) |
| 1191 | 462.47 | 463.4 | (400 MHZ, DMSO-d₆): δ8.17 (t, 1H), 7.71 (d, 1H), 7.59 (s, 1H), 7.38 - |
| | | (M + H) | 7.33 (m, 2H), 7.18 (d, 1H), 4.69 (t, 2H), 4.65 (d, 1H), 4.22 (dd, 1H), 4.13 |
| | | | (t, 2H), 3.78-3.73 (m, 1H), 3.58-3.43 (m, 3H), 3.23-3.09 (m, 2H), 3.03- |
| | | | 2.87 (m, 2H), 2.82-2.71 (m, 4H), 2.10-2.02 (m, 2H) |
| 1196 | 450.96 | 451.4 | (400 MHz, CD₃OD): δ 9.02 (s, 1H), 8.65 (dd, 1H), 8.53 (d, 1H), 7.23 (s, |
| | | (M + H) | 1H), 7.21-7.18 (m, 1H), 6.70 (s, 1H), 3.87 (dd, 1H), 3.83 (dd, 1H), 3.51- |
| | | | 3.43 (m, 1H), 3.38-3.33 (m, 1H), 3.19-3.09 (m, 3H), 2.99 (dd, 1H), 2.87- |
| | | | 2.77 (m, 1H), 2.66-2.52 (m, 1H), 2.25-2.05 (m, 2H), 1.72-1.62 (m, 2H), |
| | | | 1.59-1.44 (m, 4H), 1.41-1.32 (m, 2H). |
| 1197 | 446.55 | 447.2 | (400 MHZ, CD₃OD): δ 9.01 (s, 1H), 8.63 (dd, 1H), 8.50 (d, 1H), 6.93- |
| | | (M + H) | 6.83 (m, 2H), 6.32 (t, 1H), 3.87-3.82 (m, 2H), 3.82 (s, 3H), 3.50-3.42 (m, |
| | | | 1H), 3.37-3.33 (m, 1H), 3.17-3.09 (m, 3H), 2.99 (dd, 1H), 2.88-2.74 (m, |
| | | | 1H), 2.64-2.51 (m, 1H), 2.22-2.05 (m, 2H), 1.71-1.62 (m, 2H), 1.58-1.43 |
| | | | (m, 4H), 1.41-1.30 (m, 2H) |
| 1198 | 450.96 | 451.2 | (400 MHZ, CD₃OD): δ 8.97 (s, 1H), 8.55 (dd, 1H), 8.37 (d, 1H), 8.02 (d, |
| | | (M + H) | 1H), 7.88 (dd, 1H), 6.82 (d, 1H), 3.90-3.80 (m, 2H), 3.51-3.43 (m, 1H), |
| | | | 3.37-3.34 (m, 1H), 3.29-3.25 (m, 2H), 3.12 (t, 1H), 2.99 (dd, 1H), 2.85- |
| | | | 2.76 (m, 1H), 2.62-2.53 (m, 1H), 2.23-2.04 (m, 2H), 1.74-1.64 (m, 2H), |
| | | | 1.58-1.42 (m, 4H), 1.41-1.32 (m, 2H) |
| 1199 | 446.55 | 447.2 | (400 MHZ, CD₃OD): δ 8.99 (s, 1H), 8.53 (dd, 1H), 8.32 (d, 1H), 7.59- |
| | | (M + H) | 7.55 (m, 2H), 6.68 (d, 1H), 3.94 (s, 3H), 3.90- 3.80 (m, 2H), 3.51-3.43 |
| | | | (m, 1H), 3.37-3.34 (m, 1H), 3.21 (t, 2H), 3.12 (t, 1H), 2.99 (dd, 1H), |
| | | | 2.87-2.72 (m, 1H), 2.63-2.50 (m, 1H), 2.24-2.04 (m, 2H), 1.72-1.62 (m, |
| | | | 2H), 1.57-1.42 (m, 4H), 1.40-1.31 (m, 2H) |
| 1200 | 472.5 | 473.2 | (400 MHZ, DMSO-d₆): δ 8.15 (t, 1H), 7.70 (d, 1H), 7.35 (dd, 1H), 7.24- |
| | | (M + H) | 7.12 (m, 5H), 4.71-4.65 (m, 3H), 4.19 (dd, 1H), 3.83-3.77 (m, 1H), 3.65- |
| | | | 3.50 (m, 3H), 3.28-3.17 (m, 1H), 3.06-2.98 (m, 1H), 2.97-2.66 (m, 7H), |
| | | | 2.65-2.56 (m, 1H), 2.16 (t, 1H), 2.12 - 2.04 (m, 1H) |
| 1202 | 450.96 | 451.2 | (400 MHZ, CD₃OD): δ 9.00 (dd, 1H), 8.08-8.04 (m, 2H), 7.89 (dd, 1H), |
| | | (M + H) | 7.68 (dd, 1H), 6.85 (d, 1H), 3.90-3.80 (m, 2H), 3.51-3.43 (m, 1H), 3.37- |
| | | | 3.33 (m, 1H), 3.30-3.28 (m, 2H), 3.12 (t, 1H), 2.99 (dd, 1H), 2.87-2.76 |
| | | | (m, 1H), 2.63- 2.53 (m, 1H), 2.23-2.06 (m, 2H), 1.75-1.66 (m, 2H), 1.58- |
| | | | 1.43 (m, 4H), 1.42-1.32 (m, 2H) |
| 1203 | 439.94 | 440.1 | (400 MHZ, CD₃OD): δ 7.88-7.85 (m, 2H), 7.78 (dd, 1H), 7.20 (s, 1H), |
| | | (M + H) | 6.80 (d, 1H), 3.91-3.81 (m, 2H), 3.52-3.44 (m, 1H), 3.39-3.33 (m, 1H), |
| | | | 3.29-3.26 (m, 2H), 3.14 (t, 1H), 3.02 (dd, 1H), 2.91-2.79 (m, 1H), 2.67- |
| | | | 2.58 (m, 1H), 2.31-2.11 (m, 2H), 1.74-1.63 (m, 2H), 1.60-1.42 (m, 4H), |
| | | | 1.42-1.32 (m, 2H) |
| 1207 | 435.52 | 436.3 | (400 MHZ, CD₃OD): δ 7.84 (s, 1H), 7.51 (dd, 1H), 7.41 (d, 1H), 7.18 (s, |
| | | (M + H) | 1H), 6.63 (d, 1H), 3.92 (s, 3H), 3.87 (dd, 1H), 3.83 (dd, 1H), 3.51-3.43 |
| | | | (m, 1H), 3.35 (s, 1H), 3.20 (t, 2H), 3.13 (t, 1H), 2.99 (dd, 1H), 2.88-2.74 |
| | | | (m, 1H), 2.65-2.52 (m, 1H), 2.24-2.08 (m, 2H), 1.71-1.62 (m, 2H), 1.57- |
| | | | 1.41 (m, 4H), 1.40-1.30 (m, 2H) |
| 1211 | 439.94 | 440.1 | (400 MHZ, CD₃OD): δ 7.96 (s, 1H), 7.28 (s, 1H), 7.18-7.13 (m, 2H), |
| | | (M + H) | 6.70 (t, 1H), 3.92-3.78 (m, 2H), 3.51-3.43 (m, 1H), 3.37-3.34 (m, 1H), |
| | | | 3.16 -3.09 (m, 3H), 2.99 (dd, 1H), 2.87-2.76 (m, 1H), 2.63-2.53 (m, 1H), |
| | | | 2.22-2.07 (m, 2H), 1.72-1.60 (m, 2H), 1.58-1.43 (m, 4H), 1.40-1.32 (m, |
| | | | 2H) |
| 1212 | 450.96 | 451.4 | (400 MHZ, CD₃OD): δ8.82 (d, 2H), 7.57 (d, 2H), 7.36 (t, 1H), 6.72 (t, |
| | | (M + H) | 1H), 3.87(dd, 1H), 3.83 (dd, 1H), 3.51-3.43 (m, 1H), 3.38-3.34 (m, 1H), |
| | | | 3.19-3.09 (m, 3H), 2.99 (dd, 1H), 2.87-2.77 (m, 1H), 2.64-2.54 (m, 1H), |
| | | | 2.23-2.06 (m, 2H), 1.71-1.62 (m, 2H), 1.59-1.44 (m, 4H), 1.42-1.31 (m, |
| | | | 2H) |
| 1216 | 435.52 | 436.2 | (400 MHZ, CD₃OD): δ 7.93 (s, 1H), 7.26 (s, 1H), 6.89 (dd, 1H), 6.84 |
| | | (M + H) | (dd, 1H), 6.31 (t, 1H), 3.89-3.82 (m, 2H), 3.80 (s, 3H), 3.52-3.41 (m, 1H), |
| | | | 3.38-3.33 (m, 1H), 3.16-3.08 (m, 3H), 2.99 (dd, 1H), 2.89-2.75 (m, 1H), |
| | | | 2.66-2.51 (m, 1H), 2.22-2.04 (m, 2H), 1.70-1.61 (m, 2H), 1.58-1.43 (m, |
| | | | 4H), 1.41-1.32 (m, 2H) |
| 1217 | 446.55 | 447.2 | (400 MHZ, CD₃OD): δ 8.81 (d, 2H), 7.34 (t, 1H), 7.30 (dd, 1H), 7.24 |
| | | (M + H) | (dd, 1H), 6.35 (t, 1H), 3.89-3.79 (m, 5H), 3.50-3.42 (m, 1H), 3.37-3.34 |
| | | | (m, 1H), 3.21-3.09 (m, 3H), 2.99 (dd, 1H), 2.87-2.75 (m, 1H), 2.64-2.52 |
| | | | (m, 1H), 2.22-2.07 (m, 2H), 1.72-1.60 (m, 2H), 1.58-1.44 (m, 4H), 1.41- |
| | | | 1.31 (m, 2H) |
| 1219 | 434.54 | 435.5 | (400 MHZ, CD₃OD): δ 7.08 (s, 2H), 6.75 (s, 1H), 6.73-6.72 (m, 1H), |
| | | (M + H) | 6.22 (t, 1H), 3.85 (dd, 2H), 3.80 (s, 3H), 3.50-3.42 (m, 1H), 3.38-3.33 (m, |
| | | | 1H), 3.16-3.09 (m, 3H), 2.99 (dd, 1H), 2.86-2.76 (m, 1H), 2.62-2.53 (m, |
| | | | 1H), 2.24-2.06 (m, 2H), 1.69-1.62 (m, 2H), 1.57-1.44 (m, 4H), 1.41-1.31 |
| | | | (m, 2H) |
| 1221 | 539.63 | 540.5 | (400 MHZ, CD₃OD): δ 7.81 (d, 1H), 7.28 (dd, 1H), 7.12 (d, 1H), 3.87 |
| | | (M + H) | (dd, 1H), 3.82 (dd, 1H), 3.46-3.40 (m, 3H), 3.39-3.36 (m, 2H), 3.36-3.35 |
| | | | (m, 1H), 3.32-3.29 (m, 3H), 3.13 (t, 1H), 3.01 (dd, 1H), 2.92-2.84 (m, |
| | | | 1H), 2.72-2.39 (m, 7H), 2.14-2.00 (m, 2H), 1.95-1.81 (m, 2H), 1.75-1.64 |
| | | | (m, 2H), 1.61-1.46 (m, 4H), 1.39-1.27 (m, 2H) |
| 1226 | 519.56 | 520.2 | (400 MHZ, CD₃OD): δ9.23 (d, 1H), 8.79 (d, 2H), 8.53 (dd, 1H), 7.28 (t, |
| | | (M + H) | 1H), 7.15 (d, 1H), 3.91 (dd, 1H), 3.83 (dd, 1H), 3.54 (t, 2H), 3.49-3.41 |
| | | | (m, 1H), 3.35-3.28 (m, 1H), 3.12 (t, 1H), 3.08-3.00 (m, 2H), 2.95-2.82 |
| | | | (m, 4H), 2.76-2.69 (m, 2H), 2.62-2.55 (m, 1H), 2.43 (t, 2H), 2.21 (t, 1H), |
| | | | 2.15-2.11 (m, 1H) |
| 1227 | 535.66 | 536.1 | (400 MHZ, CD₃OD): δ 8.72 (d, 2H), 8.28 (d, 1H), 8.21 (dd, 1H), 7.20 (t, |
| | | (M + H) | 1H), 6.78 (d, 1H), 3.91-3.81 (m, 2H), 3.52-3.46 (m, 1H), 3.40-3.31 (m, |
| | | | 1H), 3.23 (t, 2H), 3.14 (t, 1H), 3.00 (dd, 1H), 2.88-2.76 (m, 1H), 2.73- |
| | | | 2.53 (m, 2H), 2.24-2.08 (m, 2H), 1.73-1.66 (m, 2H), 1.60-1.45 (m, 4H), |
| | | | 1.42-1.30 (m, 2H), 1.08-0.99(m, 4H) |
| 1228 | 450.96 | 451.6 | (400 MHZ, CD₃OD): δ8.72 (d, 2H), 8.27 (d, 1H), 8.18 (dd, 1H), 7.21 (t, |
| | | (M + H) | 1H), 6.80 (d, 1H), 3.90-3.80 (m, 2H), 3.50-3.40 (m, 1H), 3.35-3.33 (m, |
| | | | 1H), 3.30-3.26 (m, 2H), 3.12 (t, 1H), 2.99 (dd, 1H), 2.88-2.73 (m, 1H), |
| | | | 2.65-2.53 (m, 1H), 2.20-2.08 (m, 2H), 1.74-1.66 (m, 2H), 1.58-1.44 (m, |
| | | | 4H), 1.41-1.31 (m, 2H) |
| 1229 | 434.51 | 435.4 | (400 MHZ, CD₃OD): δ 8.72 (d, 2H), 8.10-8.06 (m, 1H), 7.96 (dd, 1H), |
| | | (M + H) | 7.21 (t, 1H), 6.80 (t, 1H), 3.91-3.81 (m, 2H), 3.52-3.43 (m, 1H), 3.39- |
| | | | 3.35 (m, 1H), 3.26 (t, 2H), 3.13 (t, 1H), 3.00 (dd, 1H), 2.88-2.77 (m, 1H), |
| | | | 2.64-2.54 (m, 1H), 2.24-2.07 (m, 2H), 1.74-1.66 (m, 2H), 1.60-1.44 (m, |
| | | | 4H), 1.42-1.32 (m, 2H) |
| 1231 | 446.55 | 447.5 | (400 MHZ, CD₃OD): δ 8.70 (d, 2H), 7.92 (dd, 1H), 7.82 (d, 1H), 7.18 (t, |
| | | (M+) | 1H), 6.66 (d, 1H), 3.94 (s, 3H), 3.90-3.78 (m, 2H), 3.52-3.41 (m, 1H), |
| | | | 3.38-3.34 (m, 1H), 3.23 (t, 2H), 3.12 (t, 1H), 2.99 (dd, 1H), 2.87-2.77 (m, |
| | | | 1H), 2.64-2.52 (m, 1H), 2.23-2.08 (m, 2H), 1.73-1.64 (m, 2H), 1.58-1.42 |
| | | | (m, 4H), 1.41-1.32 (m, 2H) |
| 1233 | 430.55 | 431.4 | (400 MHZ, CD₃OD): δ 8.69 (d, 2H), 8.08 (dd, 1H), 8.02 (dd, 1H), 7.17 |
| | | (M + H) | (t, 1H), 6.67 (d, 1H), 3.90-3.80 (m, 2H), 3.51-3.41 (m, 1H), 3.35-3.33 (m, |
| | | | 1H), 3.26 (t, 2H), 3.12 (t, 1H), 2.99 (dd, 1H), 2.87-2.75 (m, 1H), 2.64- |
| | | | 2.52 (m, 1H), 2.23-2.16 (m, 4H), 2.15-2.08 (m, 1H), 1.75-1.66 (m, 2H), |
| | | | 1.59-1.43 (m, 4H), 1.42-1.31 (m, 2H) |
| 1234 | 524.58 | 525.5 | (400 MHZ, CD₃OD): δ 7.78 (d, 1H), 7.27 (dd, 1H), 7.10 (d, 1H), 3.87 |
| | | (M + H) | (dd, 1H), 3.81 (dd, 1H), 3.51-3.39 (m, 3H), 3.37-3.33 (m, 1H), 3.13 (t, |
| | | | 1H), 3.00 (dd, 1H), 2.92-2.83 (m, 1H), 2.70-2.38 (m, 7H), 2.22 (t, 2H), |
| | | | 2.15-2.01 (m, 2H), 1.92-1.84 (m, 2H), 1.82-1.75 (m, 2H), 1.72-1.64 (m, |
| | | | 2H) |
| 1235 | 485.5 | 483.1 | (400 MHZ, CD₃OD): δ 7.81 (d, 1H), 7.28 (dd, 1H), 7.10 (d, 1H), 3.94- |
| | | (M + H) | 3.78 (m, 2H), 3.51-3.38 (m, 3H), 3.36-3.27 (m, 1H), 3.11 (t, 1H), 3.07- |
| | | | 2.98 (m, 2H), 2.91-2.78 (m, 4H), 2.73-2.66 (m, 2H), 2.61-2.50 (m, 1H), |
| | | | 2.41 (t, 2H), 2.19 (t, 1H), 2.15-2.10 (m, 1H) |
| 1236 | 413.53 | 414.5 | (400 MHZ, CD₃OD): δ 7.03-6.97 (m, 2H), 6.76-6.70 (m, 1H), 3.90-3.79 |
| | | (M + H) | (m, 2H), 3.71 (s, 2H), 3.51-3.43 (m, 1H), 3.38-3.34 (m, 1H), 3.21-3.09 |
| | | | (m, 3H), 2.99 (dd, 1H), 2.87-2.75 (m, 1H), 2.65-2.53 (m, 1H), 2.48 (s, |
| | | | 6H), 2.23-2.09 (m, 2H), 1.69-1.60 (m, 2H), 1.57-1.41 (m, 4H), 1.40-1.30 |
| | | | (m, 2H) |
| 1239 | 480.95 | 481.1 | (400 MHZ, CD₃OD): δ 7.21-7.11 (m, 4H), 6.99 (d, 1H), 6.83 (d, 1H), |
| | | (M + H) | 3 .. 90 (dd, 1H), 3.83 (dd, 1H), 3.53-3.45 (m, 1H), 3.40 (t, 2H), 3.36-3.33 |
| | | | (m, 1H), 3.16 (t, 1H), 3.05 (dd, 1H), 2.95-2.75 (m, 6H), 2.33 (t, 1H), |
| | | | 2.22-2.16 (m, 1H) |
| 1240 | 472.5 | 473.1 | (400 MHZ, CD₃OD): δ 7.78 (d, 1H), 7.28-7.20 (m, 2H), 7.16-7.03 (m, |
| | | (M + H) | 4H), 3.96-3.82 (m, 1H), 3.66-3.58 (m, 2H), 3.52-3.46 (m, 1H), 3.37-3.33 |
| | | | (m, 1H), 3.16 (t, 1H), 3.08 (dd, 1H), 3.01-2.92 (m, 3H), 2.91-2.86 (m, |
| | | | 1H), 2.85-2.71 (m, 2H), 2.34 (t, 1H), 2.27-2.21 (m, 1H) |
| 1245 | 439.55 | 439.5 | (400 MHZ, CD₃OD): δ 7.52 (d, 1H), 7.28 (dd, 1H), 6.72 (d, 1H), 3.89- |
| | | (M+) | 3.80 (m, 2H), 3.70-3.66 (m, 1H), 3.51 (s, 1H), 3.49-3.43 (m, 1H), 3.38- |
| | | | 3.33 (m, 1H), 3.21-3.09 (m, 3H), 2.99 (dd, 1H), 2.86-2.74 (m, 1H), 2.65- |
| | | | 2.49 (m, 1H), 2.34 (s, 6H), 2.23-2.06 (m, 2H), 1.72-1.63 (m, 2H), 1.58- |
| | | | 1.44 (m, 4H), 1.41-1.29 (m, 2H) |
| 1246 | 483.47 | 484.5 | (400 MHZ, CD₃OD): δ 9.26-9.22 (m, 1H), 8.91-8.81 (m, 3H), 7.81 (d, |
| | | (M + H) | 1H), 7.40 (t, 1H), 4.42-4.21 (m, 2H), 3.89 (dd, 1H), 3.83 (dd, 1H), 3.51- |
| | | | 3.43 (m, 1H), 3.38 - 3.33 (m, 1H), 3.13 (t, 1H), 3.00 (dd, 1H), 2.91-2.77 |
| | | | (m, 1H), 2.70 (s, 3H), 2.64-2.53 (m, 1H), 2.24-2.08 (m, 2H), 1.97-1.78 |
| | | | (m, 2H), 1.65-1.51 (m, 4H), 1.50-1.36 (m, 2H) |
| 1247 | 555.71 | 554.7 | (400 MHz, CD₃OD): δ 8.81 (d, 1H), 8.75 (d, 2H), 8.40 (dd, 1H), 7.24 (t, |
| | | (M − H) | 1H), 6.90 (d, 1H), 3.94 -3.80 (m, 2H), 3.52-3.44 (m, 1H), 3.39-3.35 (m, |
| | | | 1H), 3.14 (t, 1H), 3.02 (dd, 1H), 2.89-2.80 (m, 1H), 2.73 (m, 2H), 2.71 (s, |
| | | | 6H), 2.67-2.55 (m, 1H), 2.27-2.06 (m, 2H), 1.80-1.71 (m, 2H), 1.62-1.49 |
| | | | (m, 4H), 1.45-1.35 (m, 2H) |
| 1249 | 472.5 | 473.2 | (400 MHZ, CD₃OD): δ 7.80 (d, 1H), 7.31-7.24 (m, 2H), 7.23-7.11 (m, |
| | | (M + H) | 4H), 3.95-3.89 (m, 1H), 3.86-3.80 (m, 1H), 3.71-3.62 (m, 2H), 3.55-3.45 |
| | | | (m, 1H), 3.16 (t, 1H), 3.10-3.04 (m, 3H), 2.97-2.80 (m, 5H), 2.34 (t, 1H), |
| | | | 2.24-2.19 (m, 1H) |
| 1250 | 460.53 | 460.6 | (400 MHZ, CD₃OD): δ 8.74 (d, 2H), 8.65 (d, 1H), 8.32 (dd, 1H), 7.22 (t, |
| | | (M+) | 1H), 6.82 (d, 1H), 3.90- 3.81 (m, 2H), 3.51-3.42 (m, 1H), 3.38-3.34 (m, |
| | | | 1H), 3.27 (t, 2H), 3.12 (t, 1H), 2.99 (dd, 1H), 2.86-2.76 (m, 1H), 2.63- |
| | | | 2.54 (m, 1H), 2.21-2.07 (m, 2H), 1.76-1.67 (m, 2H), 1.59-1.46 (m, 4H), |
| | | | 1.42-1.32 (m, 2H) |
| 1251 | 381.45 | 382.1 | (400 MHZ, CD₃OD): δ 7.30 (dd, 1H), 6.44 (dd, 1H), 6.36 (dd, 1H), |
| | | (M + H) | 3.90-3.80 (m, 2H), 3.54-3.43 (m, 1H), 3.39-3.28 (m, 1H), 3.17-3.07 (m, |
| | | | 3H), 3.00 (dd, 1H), 2.87-2.76 (m, 1H), 2.64-2.53 (m, 1H), 2.24-2.07 (m, |
| | | | 2H), 1.66-1.57 (m, 2H), 1.56-1.39 (m, 4H), 1.38-1.28 (m, 2H) |
| 1252 | 424.17 | 425.1 | (400 MHZ, CD₃OD): δ 7.49 (d, 1H), 7.22 (dd, 1H), 6.81 (d, 1H), 4.44- |
| | | (M + H) | 4.37 (m, 1H), 4.18-4.12 (m, 2H), 3.95-3.91 (m, 1H), 3.85-3.78 (m, 3H), |
| | | | 3.59 (t, 2H), 3.50-3.41 (m, 1H), 3.36-3.34 (m, 1H), 3.17-3.02 (m, 3H), |
| | | | 2.79-2.91 (m, 1H), 2.29 (t, 1H), 2.24-2.18 (m, 1H) |
| 1253 | 471.9 | 472.4 | (400 MHZ, CD₃OD): δ7.65 (s, 1H), 6.88 (d, 1H), 6.64 (d, 1H), 4.50 (t, |
| | | (M + H) | 2H), 3.94-3.89 (m, 1H), 3.86-3.82 (m, 1H), 3.63 (t, 2H), 3.49-3.41 (m, |
| | | | 1H), 3.38-3.33 (m, 1H), 3.17-3.11 (m, 1H), 3.08-3.00 (m, 2H), 2.87-2.76 |
| | | | (m, 3H), 2.25 (t, 1H), 2.21-2.15 (m, 1H) |
| 1254 | 437.46 | 438.1 | (400 MHZ, CD₃OD): δ 7.42 (d, 1H), 7.38-7.35 (m, 1H), 7.29-7.26 (m, |
| | | (M + H) | 1H), 3.92-3.81 (m, 2H), 3.49-3.39 (m, 2H), 3.35-3.31 (m, 2H), 3.13 (t, |
| | | | 1H), 3.06-2.98 (m, 5H), 2.73-2.60 (m, 4H), 2.59-2.44 (m, 3H), 2.25 (t, |
| | | | 1H), 2.17-2.12 (m, 1H) |
| 1255 | 412.4 | 413 | (400 MHZ, CD₃OD): δ 8.22 (br s, 1H), 7.80 (d, 1H), 7.27 (dd, 1H), 7.13 |
| | | (M + H) | (d, 1H), 3.91 (dd, 1H), 3.81 (dd, 1H), 3.77-3.72 (m, 2H), 3.69-3.63 (m, |
| | | | 2H), 3.58-3.53 (m, 2H), 3.50-3.41 (m, 1H), 3.36-3.33 (m, 1H), 3.13 (t, |
| | | | 1H), 3.10-3.02 (m, 2H), 2.81-2.72 (m, 1H), 2.30 (t, 1H), 2.23-2.18 (m, |
| | | | 1H) |
| 1256 | 441.53 | 442.3 | (400 MHZ, CD₃OD): δ 8.76 (d, 2H), 8.48-8.41 (m, 2H), 7.26 (t, 1H), |
| | | (M + H) | 6.92-6.88 (m, 1H), 3.89 (dd, 1H), 3.84 (dd, 1H), 3.52-3.46 (m, 1H), 3.38- |
| | | | 3.34 (m, 3H), 3.13 (t, 1H), 3.00 (dd, 1H), 2.90-2.73 (m, 1H), 2.66-2.52 |
| | | | (m, 1H), 2.24-2.06 (m, 2H), 1.76-1.66 (m, 2H), 1.60-1.44 (m, 4H), 1.43- |
| | | | 1.33 (m, 2H) |
| 1257 | 420.55 | 421.4 | (400 MHZ, CD₃OD): δ 7.37 (dd, 1H), 7.33 (d, 1H), 6.76 (d, 1H), 3.88 |
| | | (M + H) | (dd, 1H), 3.83 (dd, 1H), 3.52-3.43 (m, 1H), 3.36-3.31 (m, 3H), 3.25 (t, |
| | | | 2H), 3.13 (t, 1H), 2.99 (dd, 1H), 2.86-2.76 (m, 1H), 2.63-2.53 (m, 1H), |
| | | | 2.22 (s, 6H), 2.20-2.08 (m, 2H), 1.71-1.62 (m, 2H), 1.58-1.41 (m, 4H), |
| | | | 1.41-1.32 (m, 2H) |
| 1258 | 484.52 | 485.2 | (400 MHZ, CD₃OD): δ 8.74 (d, 2H), 8.51 (d, 1H), 8.42 (dd, 1H), 7.24 (t, |
| | | (M + H) | 1H), 6.92 (d, 1H), 3.89-3.80 (m, 2H), 3.51-3.43 (m, 1H), 3.34-3.32 (m, |
| | | | 3H), 3.12 (t, 1H), 2.99 (dd, 1H), 2.86-2.76 (m, 1H), 2.63-2.53 (m, 1H), |
| | | | 2.22-2.01 (m, 2H), 1.74-1.65 (m, 2H), 1.57-1.43 (m, 4H), 1.42-1.31 (m, |
| | | | 2H) |
| 1259 | 463.54 | 464.5 | (400 MHZ, CD₃OD): δ7.40-7.28 (m, 2H), 6.79 (d, 1H), 3.89-3.79 (m, |
| | | (M + H) | 2H), 3.51-3.42 (m, 1H), 3.40 (s, 2H), 3.37-3.32 (m, 1H), 3.22 (t, 2H), |
| | | | 3.12 (t 1H), 2.98 (dd, 1H), 2.87-2.73 (m, 1H), 2.62-2.49 (m, 1H), 2.23 (s, |
| | | | 6H), 2.19-2.07 (m, 2H), 1.70-1.61 (m, 2H), 1.56- 1.41 (m, 4H), 1.40-1.28 |
| | | | (m, 2H) |
| 1260 | 422.44 | 423.4 | (400 MHZ, CD₃OD): δ 8.05 (br s, 1H), 7.79 (d, 1H), 7.26 (dd, 1H), 7.09 |
| | | (M + H) | (d, 1H), 5.66-5.49 (m, 2H), 3.90-3.81 (m, 2H), 3.50-3.32 (m, 4H), 3.12 (t, |
| | | | 1H), 2.98 (dd,1H), 2.89-2.78 (m, 1H), 2.70-2.61 (m, 1H), 2.45-2.39 (m, |
| | | | 2H), 2.28-2.17 (m, 3H), 2.16-2.10 (m, 1H) |
| 1265 | 495.6 | 496.5 | (400 MHZ, CD₃OD): δ 8.84 (d, 1H), 8.79-8.73 (m, 2H), 8.43-8.37 (m, |
| | | (M + H) | 1H), 7.27-7.20 (m, 1H), 7.01-6.83 (m, 1H), 3.93-3.81 (m, 2H), 3.53-3.44 |
| | | | (m, 1H), 3.40-3.33 (m, 2H), 3.31-3.26 (m, 1H), 3.15 (t, 1H), 3.06-2.98 |
| | | | (m, 1H), 2.90-2.79 (m, 1H), 2.73-2.68 (m, 1H), 2.67-2.55 (m, 1H), 2.26- |
| | | | 2.11 (m, 2H), 1.81-1.67 (m, 2H), 1.59-1.48 (m, 4H), 1.44-1.34 (m, 2H) |
| 1266 | 459.55 | 460.3 | (400 MHZ, CD₃OD): δ 8.74 (d, 2H), 8.64 (d, 1H), 8.32 (dd, 1H), 7.21 (t, |
| | | (M + H) | 1H), 6.82 (d, 1H), 3.90 -3.80 (m, 2H), 3.51-3.42 (m, 1H), 3.37-3.32 (m, |
| | | | 1H), 3.27 (t, 2H), 3.12 (t, 1H), 2.99 (dd, 1H), 2.87-2.76 (m, 1H), 2.64- |
| | | | 2.53 (m, 1H), 2.22-2.06 (m, 2H), 1.76-1.67 (m, 2H), 1.60-1.45 (m, 4H), |
| | | | 1.43-1.31 (m, 2H) |
| 1268 | 455.43 | 456.1 | (400 MHZ, CD₃OD): δ7.80 (d, 1H), 7.29-7.24 (m, 1H), 7.21-7.17 (m, |
| | | (M + H) | 1H), 4.38-4.28 (m, 2H), 3.95-3.90 (m, 1H), 3.86-3.81 (m, 1H), 3.64 (t, |
| | | | 2H), 3.48-3.41 (m, 1H), 3.29-3.26 (m, 2H), 3.14 (t, 2H), 3.04 (dd, 1H), |
| | | | 3.00-2.90 (m, 1H), 2.59-2.52 (m, 1H), 2.26-2.10 (m, 2H) |
| 1270 | 456.46 | 457.3 | (400 MHZ, CD₃OD): δ 7.80 (d, 1H), 7.27 (dd, 1H), 7.14 (d, 1H), 3.98- |
| | | (M + H) | 3.80 (m, 2H), 3.63-3.34 (m, 6H), 3.22-3.01 (m, 3H), 2.70-2.49 (m, 1H), |
| | | | 2.22-2.05 (m, 2H), 1.98-1.57 (m, 4H) |
| 1271 | 467.48 | 468.4 | (400 MHZ, CD₃OD): δ7.81 (d, 1H), 7.27 (dd, 1H), 7.09 (d, 1H), 4.21- |
| | | (M + H) | 4.14 (m, 1H), 3.94-3.89 (m, 1H), 3.85-3.79 (m, 1H), 3.72-3.65 (m, 2H), |
| | | | 3.53-3.49 (m, 2H), 3.48-3.32 (m, 4H), 3.13 (t, 1H), 3.08-2.98 (m, 4H), |
| | | | 2.81 (t, 2H), 2.74-2.66 (m, 1H), 2.27 (t, 1H), 2.22-2.16 (m, 1H) |
| 1272 | 463.46 | 464.4 | (400 MHz, CD₃OD): δ 7.81-7.77 (m, 2H), 7.22 (dd, 1H), 6.98 (d, 1H), |
| | | | 4.65 (t, 2H), 3.94-3.88 (m, 3H), 3.86-3.80 (m, 1H), 3.50-3.42 (m, 1H), |
| | | | 3.17-3.12 (m, 1H), 3.08-3.01 (m, 2H), 2.89-2.76 (m, 4H), 2.24 (t, 1H), |
| | | | 2.21-2.15 (m, 1H) |
| 1273 | 480.3 | 481.2 | (400 MHZ, CD₃OD): δ 7.81 (d, 1H), 7.27 (dd, 1H), 7.08 (d, 1H), 3.91- |
| | | (M + H) | 3.86 (m, 1H), 3.86-3.81 (m, 1H), 3.51-3.41 (m, 3H), 3.38-3.36 (m, 1H), |
| | | | 3.35-3.32 (m,2H), 3.13 (t, 1H), 3.07-2.93 (m, 2H), 2.74-2.70 (m, 2H), |
| | | | 2.68-2.43 (m, 9H), 2.25 (t, 1H), 2.18-2.12 (m, 1H) |
| 1274 | 474.49 | 475 | (400 MHZ, CD₃OD): δ 7.83-7.82 (m, 1H), 7.35-7.30 (m, 1H), 7.17-7.10 |
| | | (M + H) | (m, 1H), 3.98-3.85 (m, 3H), 3.84-3.77 (m, 1H), 3.48-3.43 (m, 3H), 3.36- |
| | | | 3.32 (m, 1H), 3.27-3.1 (m, 3H), 3.01-2.9 (m, 2H), 2.66-2.58 (m, 1H), |
| | | | 2.19-2.12 (m, 2H), 2.03-1.92 (m, 2H). |
| 1275 | 490.49 | 491.1 | (400 MHZ, CD₃OD): δ 7.82 (d, 1H), 7.28 (dd, 1H), 7.16 (d, 1H), 3.91- |
| | | (M + H) | 3.81 (m, 2H), 3.61-3.53 (m, 2H), 3.49-3.40 (m, 1H), 3.37-3.32 (m, 1H), |
| | | | 3.26 (t, 2H), 3.18-2.99 (m, 5H), 2.62-2.51 (m, 1H), 2.23-2.07 (m, 2H) |
| 1276 | 454.44 | 455.2 | (400 MHZ, CD₃OD): δ 7.80 (d, 1H), 7.29-7.24 (m, 1H), 7.21-7.17 (m, |
| | | (M + H) | 1H), 3.95-3.90 (m, 1H), 3.86-3.81 (m, 1H), 3.50-3.41 (m, 5H), 3.39-3.34 |
| | | | (m, 1H), 3.29-3.26 (m, 1H), 3.25-3.19 (m, 1H), 3.14 (t, 1H), 3.04 (dd, |
| | | | 1H), 3.00-2.90 (m, 1H), 2.56-2.48 (m, 1H), 2.26-2.10 (m, 2H) |
| 1277 | 460.96 | 461.1 | (400 MHZ, CD₃OD): δ 6.99-6.92 (m, 1H), 6.8-6.70 (m, 1H), 3.93-3.8 |
| | | (M + H) | (m, 2H), 3.54-3.43 (m, 1H), 3.42-3.30 (m, 1H), 3.20-3.13 (m, 3H), 3.04- |
| | | | 2.9 (m, 1H), 2.82-2.72 (m, 1H), 2.63-2.52 (m, 1H), 2.19-2.06 (m, 2H), |
| | | | 1.64-1.57 (m, 2H), 1.56-1.42 (m, 2H), 1.41-1.19 (m, 8H). |
| 1280 | 454.44 | 455.2 | (400 MHZ, CD₃OD): δ 8.19 (t, 1H), 7.81 (d, 1H), 7.26 (dd, 1H), 7.12 (d, |
| | | (M + H) | 1H), 3.98-3.85 (m, 3H), 3.85-3.73 (m, 4H), 3.54-3.35 (m, 4H), 3.35-3.33 |
| | | | (m, 1H), 3.13 (t, 1H), 3.09-3.04 (m, 1H), 2.89-2.80 (m, 1H), 2.57-2.43 |
| | | | (m, 1H), 2.31 (t, 1H), 2.27-2.14 (m, 1H) |
| 1288 | 437.50 | | (400 MHZ, CD₃OD): δ 8.3 (brs, 2 H), 7.55 (s, 1 H), 7.15-7.05 (m, 1H), |
| | | | 6.85-6.80 (m, 1H), 4.20-4.10 (m, 1H), 3.95-3.90 (m,1H), 3.85 (m, 3H), |
| | | | 3.70-3.60 (m, 2H), 3.45-3.40 (m, 1H), 3.30-3.25 (m, 3H), 3.20-3.10 (m, |
| | | | 1H), 2.90-2.80 (m, 2H), 1.85-1.65 (m, 4H), 1.60-1.40 (m, 4H). |
| 1290 | 421.49 | 422.4 | (400 MHZ, CD₃OD): δ 8.09-8.02 (m, 1H), 7.48-7.40 (m, 1H), 6.99-6.90 |
| | | (M + H) | (m, 1H), 4.18-4.06 (m, 1H), 3.93-3.82 (m, 1H), 3.80-3.59 (m, 2H), 3.50- |
| | | | 3.37 (m, 5H), 3.25-3.18 (m, 1H), 3.10-2.92 (m, 2H), 2.05-1.94 (m, 3H), |
| | | | 1.91-1.7 (m, 4H), 1.6-1.35 (m, 4H) |
| 1291 | 469 | 470 | (400 MHZ, CD₃OD): δ 8.10 (s, 1H), 7.45-7.40 (m, 1H), 6.95-6.90 |
| | | (M + H) | (m,1H), 3.90-3.85 (m, 2H), 3.56-3.35 (m, 2H), 3.50-3.45 (m, 2H), 3.45- |
| | | | 3.42 (m, 1H), 3.15-3.10 (m, 1H), 3.10-3.05 (m, 1H), 2.90-2.85 (m, 1H), |
| | | | 2.70-2.60 (m, 1H), 2.30-2.20 (m, 2H), 1.75-1.65 (m, 2H), 1.60-1.50 (m, |
| | | | 4H), 1.42-1.35 (m, 2H), 1.22-1.15 (m, 3H) |
| 1292 | 427.5 | 428.7 | (400 MHZ, CD₃OD): δ 8.01-7.99 (m, 1H), 7.47-7.38 (m, 1H), 6.97-6.93 |
| | | (M + H) | (m, 1H), 4.15-4.07 (m, 1H), 3.90-3.83 (m, 1H), 3.80-3.71 (m, 2H), 3.69- |
| | | | 3.58 (m, 1H), 3.57-3.52 (m, 1H), 3.51-3.32 (m, 6H), 3.26-3.17 (m, 1H), |
| | | | 3.13-2.90 (m, 2H), 2.8-2.71 (m, 2H), 1.86-1.68 (m, 4H), 1.58-1.42 (m, |
| | | | 4H). |
| 1296 | 422.49 | 423.4 | (400 MHz, CD₃OD): δ 7.36-7.34 (m, 1H), 7.14-7.09 (m, 1H), 6.92-6.87 |
| | | (M + H) | (m, 1H), 4.16-4.09 (m, 1H), 3.98-3.97 (m, 1H), 3.9-3.84 (m, 1H), 3.70- |
| | | | 3.61 (m, 1H), 3.60-3.54 (m, 1H), 3.50-3.47 (m, 1H), 3.40-3.31 (m, 2H), |
| | | | 3.26-3.18 (m, 2H), 3.09-2.91 (m, 2H), 1.92-1.69 (m, 4H), 1.6-1.4 (m, 4H) |
| 1297 | 423.47 | | (400 MHZ, CD₃OD): δ 7.55 (s, 1 H), 7.10-7.05 (m, 1H), 6.80-6.70 |
| | | | (m,1H), 4.20-4.10 (m, 1H), 3.95-3.85 (m, 1H), 3.70-3.50 (m, 2H), 3.45- |
| | | | 3.35 (m, 1H), 3.30-3.25 (m, 2H), 3.20-3.10 (m, 3H), 3.05-2.90 (m, 2H), |
| | | | 1.90-1.70 (m, 4H), 1.60-1.40 (m, 4H). |
| 1299 | 422.48 | 423.4 | (400 MHz, CD₃OD): δ 8.15 (s, 1H), 7.54-7.50 (m, 1H), 7.10-7.00 (m, |
| | | (M + H) | 1H), 3.90-3.85 (m, 2H), 3.85-3.80 (m, 2H), 3.55-3.45 (m, 1H), 3.50-3.40 |
| | | | (m,3H), 3.20-3.10 (m, 1H), 3.10-3.00 (m, 1H), 2.85-2.80 (m, 1H), 2.70- |
| | | | 2.60 (m, 1H), 2.30-2.20 (m, 2H), 1.80-1.70 (m, 2H), 1.60-1.50 (m, 4H), |
| | | | 1.40-1.30 (m, 2H). |
| 1300 | 476.55 | | (500 MHZ, CD₃OD): δ 8.10 (s, 1H), 7.50-7.54 (m, 1H), 7.10-7.00 (m, |
| | | | 1H), 3.90-3.85 (m, 2H), 3.60-3.50 (m, 1H), 3.50-3.40 (m,4H), 3.20-3.15 |
| | | | (m,1H), 3.10-3.05 (m, 1H), 2.95 (s, 3H), 2.90-2.85 (m, 1H), 2.70-2.60 |
| | | | (m, 1H), 2.25-2.15 (m, 1H), 1.80-1.70 (m, 2H), 1.50-1.40 (m, 4H), 1.40- |
| | | | 1.30 (m, 2H). |
| 1304 | 408.46 | 409.6 | (400 MHZ, CD₃OD): δ 8.60 (s, 1H), 7.65-7.75 (m, 1H), 7.10-7.15 (m, |
| | | (M + H) | 1H), 3.85-3.90 (m, 2H), 3.45-3.55 (m, 3H), 3.30-3.35 (m, 1H), 3.15-3.20 |
| | | | (m,1H), 2.95-3.00 (m, 1H), 2.85-2.90 (m, 1H), 2.60-2.65 (m, 1H), 2.15- |
| | | | 2.25 (m, 2H), 1.70-1.80 (m, 2H), 1.60-1.40 (m, 4H), 1.25-1.35 (m, 2H). |
| 1305 | 387.48 | 388 | (400 MHZ, DMSO-d₆, D₂O exchange): δ 7.60-7.50 (m, 4H), 7.30-7.25 |
| | | (M + H) | (t, 2H), 7.00-6.90 (t, 2H), 3.95-3.50 (m, 6H), 3.45-3.25 (m, 5H), 3.05- |
| | | | 2.80 (m, 2H), 2.65-2.55 (m, 2H), 2.10-1.80 (m, 2H). |
| 1306 | 359.42 | 360 | (500 MHZ, DMSO-d₆, D₂O exchange): δ 7.60-7.50 (m, 4H), 7.40-7.35 |
| | | (M + H) | (m, 2H), 7.00 (m, 2H), 4.20 (d, 1H), 3.95 (d, 1H), 3.80 (s, 3H), 3.70 (m, |
| | | | 1H), 3.20-3.10 (m, 3H), 2.95 (t, 1H), 2.70-2.60 (m, 1H), 2.00 (m, 1H). |
| 1307 | 367.49 | 368 | (400 MHZ, CD₃OD): δ 7.10 (m, 4H), 3.80 (s, 2H), 3.60 (t, 2H), 3.50-3.40 |
| | | (M + H) | (m, 1H), 3.39-3.30 (m, 3H), 3.20 (t, 1H), 3.10-3.00 (m, 1H), 2.90-2.75 |
| | | | (m, 2H), 2.70-2.50 (m, 3H), 2.35-2.20 (m, 2H), 1.70-1.50 (m, 4H), 1.40- |
| | | | 1.25 (m,2H). |
| 1308 | 339.43 | 340 | (400 MHZ, CD₃OD): δ 7.10 (m, 4H), 3.80 (s, 2H), 3.60 (t, 2H), 3.50-3.40 |
| | | (M + H) | (t, 1H), 3.39-3.30 (m, 3H), 3.20 (t, 1H), 3.00-2.95 (m, 1H), 2.90-2.85 (m, |
| | | | 2H), 2.70-2.50 (m, 3H), 2.20-2.10 (m, 2H), 1.80 (m, 2H). |
| 1309 | 367.45 | 368 | (400 MHZ, CD₃OD): δ 7.10-7.05 (d, 2H), 6.8 (d, 2H), 3.85 (s, 2H), 3.75 |
| | | (M + H) | (s, 3H), 3.5-3.40 (m, 1H), 3.40-3.30 (t, 1H), 3.15-3.10 (t, 1H), 3.05-3.00 |
| | | | (dd, 1H), 2.90-2.80 (m, 1H), 2.65-2.55 (m, 1H), 2.55-2.50 (t, 2H), 2.32- |
| | | | 2.15 (m, 2H), 1.65-1.45 (m, 4H), 1.40-1.20 (m,6H). |
| 1310 | 339.43 | 340 | (500 MHZ, CD₃OD): 7.08 (d, 2H), 6.90 (d, 2H), 3.83 (s, 2H), 3.75 (s, |
| | | (M + H) | 3H), 3.5 (m, 1H), 3.40-3.30 (m, 2H), 3.15 (t, 1H), 3.10-3.00 (m, 2H), |
| | | | 2.84-2.80 (m, 1H), 2.70-2.50 (m, 3H), 2.30-2.20 (m, 2H), 1.70-1.50 (m, |
| | | | 2H), 1.35-1.25 (m,2H). |
| 1311 | 360.5 | 361.50 | (500 MHZ, CD₃OD): δ 4.15-4.00 (m, 2H), 3.90-3.80 (m, 1H), 3.80-3.70 |
| | | (M + H) | (m, 1H), 3.70-3.65 (m,4H), 3.45-3.40 (m, 1H), 3.40-3.30 (m, 3H), 3.30- |
| | | | 3.20 (m, 3H), 3.15-2.95 (m, 3H), 2.90-2.85 (m, 1H), 1.85-1.70 (m, 2H), |
| | | | 1.60-1.30 (m, 10H). |
| 1312 | 319.44 | 320.3 | (500 MHz, CD₃OD): 4.21-4.19 (m, 1H), 4.02-3.90 (m, 5H), 3.72-3.62 |
| | | (M + H) | (m, 1H), 3.55-3.45 (m, 1H), 3.41-3.35 (m, 4H), 3.32-3.30 (s, 3H), 1.82- |
| | | | 1.76 (m, 2H), 1.62-1.55 (m, 2H), 1.45-1.23 (m, 10H). |
| 1315 | 439.51 | 440.2 | (400 MHz, CD₃OD): δ 7.90 (s, 1H), 7.36 (d, 2H), 7.31 (d, 2H), 7.23 (s, |
| | | (M + H) | 1H), 7.11-7.06 (m, 2H), 6.60 (s, 1H), 4.35 (s, 2H), 4.22 (d, 1H), 4.08 (dd, |
| | | | 1H), 3.92 (dd, 1H), 3.42-3.35 (m, 2H), 3.26 (d, 1H), 3.11 (t, 1H), 2.86 |
| | | | (dd, 1H), 2.27 (s, 3H), 2.16-2.10 (m, 1H), 1.88 (t, 1H) |
| 1316 | 439.52 | 440 | (400 MHZ, CD₃OD): δ 7.92 (s, 2H), 7.74 (s, 1H), 7.65 (s, 1H), 7.62-7.55 |
| | | (M + H) | (m, 4H), 7.03 (s, 1H), 4.84-4.83 (m, 1H), 4.65 (s, 2H), 4.29-4.10 (m, 3H), |
| | | | 3.68-3.50 (m, 2H), 3.35-3.32 (m, 1H), 3.15-3.09 (m, 2H), 2.85 (t, 1H), |
| | | | 2.42 (s, 3H) |
| 1317 | 438.22 | 439.5 | (400 MHZ, CD₃OD): δ 7.46 (dd, 1H), 7.36 (d, 2H), 7.31 (d, 2H), 6.80- |
| | | (M + H) | 6.77 (m, 2H), 6.57 (dd, 1H), 6.44 (dd, 1H), 6.40 (s, 1H), 4.32 (s, 2H), |
| | | | 4.22 (d, 1H), 4.09 (dd, 1H), 3.93 (dd, 1H), 3.41-3.36 (m, 2H), 3.26 (d, |
| | | | 1H), 3.11 (t, 1H), 2.87 (dd, 1H), 2.23 (s, 3H), 2.15-2.10 (m, 1H), 1.88 (t, |
| | | | 1H) |
| 1319 | 452.52 | 453.1 | (400 MHZ, CD₃OD): δ 8.78 (d, 2H), 8.73-8.59 (m, 2H), 7.52-7.47 (m, |
| | | (M + H) | 2H), 7.31 (t, 1H), 6.66 (s, 1H), 4.58 (s, 2H), 4.29 (d, 1H), 4.10 (dd, 1H), |
| | | | 3.92 (dd, 1H), 3.65 (d, 1H), 3.47-3.36 (m, 2H), 3.18-3.07 (m, 1H), 2.85 |
| | | | (dd, 1H), 2.32 (s, 3H), 2.30-2.22 (m, 1H), 2.17-2.08 (m, 1H) |
| 1322 | 451.53 | 452 | (400 MHZ, CD₃OD): δ 8.78 (d, 2H), 7.76 (t, 1H), 7.52-7.39 (m, 3H), |
| | | (M + H) | 7.38-7.28 (m, 2H), 6.63 (s, 1H), 4.52 (s, 2H), 4.36 (d, 1H), 4.16 (dd, 1H), |
| | | | 3.99 (dd, 1H), 3.86-3.77 (m, 1H), 3.53-3.44 (m, 2H), 3.18 (t, 1H), 2.99 |
| | | | (dd, 1H), 2.56-2.44 (m, 1H), 2.37-2.28 (m, 4H) |
| 1323 | 439.52 | 440.1 | (400 MHZ, CD₃OD): δ 8.53 (d, 1H), 7.93 (d, 1H), 7.65-7.50 (m, 4H), |
| | | (M + H) | 7.36-7.20 (m, 2H), 7.00 (s, 1H), 4.84-4.82 (m, 1H), 4.61 (s, 2H), 4.29- |
| | | | 4.10 (m, 3H), 3.70-3.51 (m, 2H), 3.38-3.34 (m, 1H), 3.19-3.09 (m, 2H), |
| | | | 2.86 (t, 1H), 2.41 (s, 3H) |
| 1324 | 438.52 | 439.5 | (400 MHZ, CD₃OD): δ 7.41-7.24 (m, 7H), 6.48 (d, 1H), 6.42-6.38 (m, |
| | | (M + H) | 2H), 4.42 (s, 2H), 4.22 (d, 1H), 4.08 (dd, 1H), 3.93 (dd, 1H), 3.43-3.33 |
| | | | (m, 2H), 3.25 (d, 1H), 3.12 (t,1H), 2.86 (dd, 1H), 2.23 (s, 3H), 2.14-2.10 |
| | | | (m, 1H), 1.88 (t, 1H) |
| 1327 | 440.5 | 441 | (400 MHZ, CD₃OD): δ 8.68 (s, 1 H) 8.60 (s, 1 H) 7.47 (dd, 1 H) 6.83 (s, |
| | | (M + H) | 1 H) 6.78 (s, 1 H) 6.61 (dd, 1 H) 6.45 (dd, , 1 H) 6.41 (s, 1 H) 4.52 (s, 2 |
| | | | H) 4.30 (d, 1 H) 4.11 (dd, 1 H) 3.91 (dd, 1 H) 3.64 (d, 1 H) 3.46- 3.39 |
| | | | (m, 2 H) 3.12 (t, 1 H) 2.85 (dd, 1 H) 2.27-2.21 (m, 4 H) 2.12 (t, 1 H) |
| 1329 | 478.55 | 479.3 | (400 MHZ, CD₃OD): δ 8.78 (d, 2 H) 8.68 (s, 1 H) 8.61 (s, 1 H) 7.47- |
| | | (M + H) | 7.45 (m, 1 H) 7.41-7.39 (m, 1 H) 7.31 (t, 1 H) 6.57 (t, 1 H) 4.57 (s, 2 H) |
| | | | 4.29 (d, 1 H) 4.11 (dd, 1 H) 3.91 (dd, 1 H) 3.64 (d, 1 H) 3.45-3.36 (m, 2 |
| | | | H) 3.11 (t, 1 H) 2.85 (dd, 1 H) 2.27-2.21 (m, 1 H) 2.12 (t, 1 H) 1.93-1.83 |
| | | | (m, 1 H) 0.98-0.89 (m, 2 H) 0.73-0.65 (m, 2 H) |
| 1330 | 439.52 | 440.1 | (400 MHZ, CD₃OD): δ 8.67 (s, 1 H) 8.61 (s, 1 H) 6.76-6.71 (m, 2 H) |
| | | (M + H) | 6.68 (d, 1 H) 6.36-6.30 (m, 2 H) 6.09 (dd, 1 H) 4.52 (s, 2 H) 4.29 (d, |
| | | | 1 H) 4.10 (dd, 1 H) 3.91 (dd, 1 H) 3.64 (d, 1 H) 3.45-3.38 (m, 2 H) 3.12 |
| | | | (t, 1 H) 2.85 (dd, 1 H) 2.27-2.24 (m, 1 H) 2.23 (s, 3 H) 2.12 (t, 1 H) |
| 1332 | 459.55 | 460.1 | (400 MHz, CD₃OD): δ 8.65 (s, 1 H) 8.57 (s, 1 H) 6.14 (s, 1 H) 6.08-6.04 |
| | | (M + H) | (m, 2 H) 4.47 (s, 2 H) 4.29 (d, 1 H) 4.11 (dd, 1 H) 3.92 (dd, 1 H) 3.80- |
| | | | 3.76 (m, 4 H) 3.64 (d, 1 H) 3.46-3.37(m, 2 H) 3.12 (t, 1 H) 3.05-3.00 (m, |
| | | | 4 H) 2.83 (dd, 1 H) 2.27-2.21 (m, 1 H) 2.16 (s, 3 H) 2.11 (t, 1 H) |
| 1333 | 477.57 | 478.0 | (400 MHZ, CD₃OD): δ 8.78 (d, 2 H) 7.72 (t, 1 H) 7.45 (dd, 1 H) 7.39- |
| | | (M + H) | 7.29 (m, 4 H) 6.52 (t, 1 H) 4.50 (s, 2 H) 4.25 (d, 1 H) 4.15 (dd, 1 H) 3.95 |
| | | | (dd, 1 H) 3.62 (d, 1 H) 3.48-3.38 (m, 2 H) 3.14 (t, 1 H) 2.89 (dd, 1 H) |
| | | | 2.32-2.27 (m, 1 H) 2.12 (t, 1 H) 1.91-1.81 (m, 1 H) 0.97-0.87 (m, 2 H) |
| | | | 0.70-0.64 (m, 2 H) |
| 1334 | 439.51 | 440.1 | (400 MHZ, CD₃OD): δ 7.73 (t, 1 H) 7.48-7.46 (m, 1 H) 7.39-7.32 (m, 2 |
| | | (M + H) | H) 6.81 (s, 1 H) 6.75 (s, 1 H) 6.59 (d, 1 H) 6.44 (dd, 1 H) 6.38 (s, 1 H) |
| | | | 4.45 (s, 2 H) 4.25 (d, 1 H) 4.15 (dd, 1 H) 3.95 (dd, 1 H) 3.61 (d, 1 H) |
| | | | 3.48- 3.40 (m, 2 H) 3.15 (t, 1 H) 2.89 (dd, 1 H) 2.32-2.27 (m, 1 H) 2.23 |
| | | | (s, 3 H) 2.12 (t, 1 H) |
| 1335 | 438.53 | 439.1 | (400 MHZ, CD₃OD): δ 7.72 (t, 1 H) 7.38 (d, 1 H) 7.33 (d, 1 H) 6.75-6.70 |
| | | (M + H) | (m, 2 H) 6.65 (s, 1 H) 6.33 (dd, 1 H) 6.29 (s, 1 H) 6.09 (dd, 1 H) 4.45 (s, |
| | | | 2 H) 4.24 (d, 1 H) 4.14 (dd, 1 H) 3.95 (dd, 1 H) 3.61 (d, 1 H) 3.38-3.50 |
| | | | (m, 2 H) 3.14 (t, 1 H) 2.88 (dd, 1 H) 2.32-2.27 (m, 1 H) 2.22 (s, 3 H) |
| | | | 2.12 (t, 1 H) |
| 1336 | 458.56 | 459.1 | (400 MHZ, CD₃OD): δ 7.74 (t, 1 H) 7.39-7.32 (m, 2 H) 6.13 (s, 1 H) |
| | | (M + H) | 6.08-6.00 (m, 2 H) 4.41 (s, 2 H) 4.30 (d, 1 H) 4.13 (dd, 1 H) 3.96 (dd, 1 |
| | | | H) 3.82-3.72 (m, 5 H) 3.51-3.39 (m, 2 H) 3.16 (t, 1 H) 3.05-2.98 (m, 4 H) |
| | | | 2.93 (dd, 1 H) 2.44-2.40 (m, 1 H) 2.27-2.18 (m, 1 H) 2.16 (s, 3 H) |
| 1337 | 440.5 | 441.1 | (400 MHZ, CD₃OD): δ 7.92 (s, 1 H) 7.73 (t, 1 H) 7.38-7.32 (m, 2 H) 7.22 |
| | | (M + H) | (s, 1 H) 7.14-7.04 (m, 1 H) 6.60 (s, 1 H) 4.47 (s, 2 H) 4.25 (d, 1 H) 4.15 |
| | | | (dd, 1 H) 3.94 (dd, 1 H) 3.62 (d, 1 H) 3.46-3.41 (m, 2 H) 3.14 (t, 1 H) |
| | | | 2.88 (dd, 2 H) 2.35-2.24 (m, 4 H) 2.12 (t, 1 H) |
| 1338 | 363.45 | 364 | (400 MHZ, CD₃OD): δ 7.30-7.20 (d, 2H), 6.85-6.80 (d, 2H), 3.85 (s, |
| | | (M + H) | 2H), 3.75 (s, 3H), 3.55-3.45 (m, 1H), 3.40-3.25 (m, 1H), 3.15-3.10 (t, |
| | | | 1H), 3.05-3.00 (dd, 1H), 2.95-2.85 (m, 1H), 2.75-2.65 (m, 1H), 2.45-2.35 |
| | | | (t, 2H), 2.35-2.25 (m, 2H), 1.70-1.55 (m,4H), 1.52-1.40 (m, 2H). |
| 1339 | 455.56 | 456.3 | (400 MHZ, CD₃OD): δ 8.83 (d,2H), 8.64 (d, 1H), 8.37 (dd, 1H), 7.57 (d, |
| | | (M + H) | 1H), 7.32 (t, 1H), 4.27 (t,2H), 3.86-3.77 (m, 2H), 3.52-3.38 (m, 1H), |
| | | | 3.37-3.32 (m, 1H), 3.10 (t, 1H), 2.95 (dd, 1H), 2.83-2.71 (m, 1H), 2.64 (s, |
| | | | 3H), 2.58-2.47 (m, 1H), 2.18-2.04 (m, 2H), 1.91-1.82 (m, 2H), 1.55-1.29 |
| | | | (m, 6H) |

### Example 119: Glucosidase Assays

Preparation of Human Full-length Glucosidase I: The amino acid sequence for human full-length glucosidase I was obtained from UniProt (Q13724). Constructs were cloned into GATEWAY entry vector, consisting of the full-length open reading frame (ORF) preceded by a Kozac sequence with a 6-His affinity tag added to the C-terminus (SEQ ID NO: 1). The entry vector was cloned into Kemp Proteins' BacMam Destination vector and transformed into chemically competent DH10Bac E. coli cells to produce recombinant glucosidase I bacmids. Bacmid isolates were obtained following two rounds of blue-white screening and they were transfected into Sf9 cells cultivated in serum-free medium (Thermo Fisher Scientific). Four days post-transfection the culture supernatants containing virus were harvested and filter sterilized. The virus titer was determined using a plaque assay on Sf9 cell monolayers and expressions were performed using HEK-293T cells cultivated in serum-free Feestyle-293 medium (Thermo Fisher). A multiplicity of infection (MOI, ratio of virus to cells) of 4 was selected. Soluble glucosidase I was detected by anti-His western blots in the soluble cell extract following extraction with 1% V/V NP40 for 30 minutes at 0° C. Productions and purifications of glucosidase I were carried out at scales ranging from 1-liter to 10-liters. In one example, production at the 1-liter scale was performed in shake-flasks at 27° C. and 100 rpm using HEK-293T cells cultivated in Freestyle-293 medium under serum-free conditions. In another example production at the 10-liter scale was performed in stirred-tank bioreactors at 27° C. and 80 rpm with dissolved oxygen level at 50% of oxygen in air and pH between 7.0 and 7.2. Cells were transduced at MOI of 4 and the cell pellet was harvested at 48 hours post-transduction using centrifugation.

The pellet from a 10-liter culture was lysed by resuspending the pellet into 1-liter of 50 mM NaH₂PO₄—H₂O+300 mM NaCl+10 mM Imidazole+1 mM PMSF+1% V/V NP40 pH 8. The suspension was incubated on ice for 30 minutes and clarified at 500×g and 4° C. The supernatant was collected and clarified at 20,000×g for 30 minutes at 4° C. and the supernatant was collected and filtered through a 0.2 micron filter. The lysate was loaded onto a 25 mL Ni-NTA Superflow (Qiagen) column (26×60 mm) at a flow rate of 10 mL per minute. The column was washed with 20 column volumes (CV) of 50 mM NaH₂PO₄-H₂O+300 mM NaCl+10 mM Imidazole pH 8 and eluted using a linear gradient of 0-60% 50 mM NaH₂PO₄-H₂O+300 mM NaCl+500 mM Imidazole pH 8 over 90 minutes. at 5 mL per minute. Fractions (25 mL) were collected and analyzed by SDS PAGE and Western Blot (reducing anti-His). Fractions containing purified glucosidase I were pooled and dialyzed into storage buffer (20 mM Tris+300 mM NaCl+50 mM L-arginine+10 mM EDTA+0.01% V/V Tween 80 pH 7.5). The final material was filter sterilized and stored at 4° C. and the working dilution confirmed by a use-test in the indicated assay conditions prior to use.

Alpha-glucosidase I Assay: The assay was performed by incubating recombinant alpha-glucosidase I enzyme with multiple dilutions of test compounds for 60 minutes at 37° C. A synthetic trisaccharide substrate analog was then introduced to the mixture for 90 minutes at 37° C. The reaction was then stopped with 1.5M Tris (pH 8). In the absence of inhibition, the terminal glucose of the substrate was hydrolyzed by the enzyme. The D-glucose product was detected using Amplex Red Glucose/Glucose Oxidase Assay Kit (Invitrogen) following the manufacturer's directions where glucose oxidase reacts with D-glucose to form D-gluconolactone and H₂O₂ which then reacts with the Amplex Red reagent to generate a red-fluorescent oxidation product (560 nm, excitation; 590 nm, emission). Compounds inhibiting glucosidase I inhibit the cleavage of the substrate and result in lower signals. Percent inhibition is plotted as a function of concentration for each compound, compared to control reactions. The IC₅₀ was determined using a 4-PL curve fit and serves as a measure of relative inhibitory activity of each test compound (see Table 3).

Preparation of Murine Full-length Glucosidase II: Glucosidase is a heterodimeric protein consisting of alpha and beta subunits. The amino acid sequence for the alpha (SEQ ID NO: 2) and beta (SEQ ID NO: 3) subunits of full-length murine glucosidase II are provided. A single construct was designed for each of the subunits for gene synthesis and cloning into Gateway entry vectors. The alpha subunit was appended with a Strep-affinity tag and the beta subunit with a 6-His affinity tag. The entry vectors were cloned into Kemp Protein's BacMam Destination vector and the vectors were transformed into chemically competent DH10Bac E. coli cells to produce recombinant glucosidase II bacmids. Bacmid isolates for each of the subunits were obtained following two rounds of blue-white screening and they were transfected into Sf9 cells cultivated in serum-free medium (Thermo Fisher Scientific). MOI of 10 with a ratio of 75% alpha subunit and 25% beta subunit was selected for expression of glucosidase II. Glucosidase II was detected in the culture supernatant and the proteins were detected using Western Blots probed with anti-His and anti-Strep antibodies. Productions and purifications of glucosidase II were carried out at scales ranging from 1-liter to 10-liters. In one example, production at the 1-liter scale was performed in shake-flasks at 27° C. and 100 rpm using HEK-293T cells cultivated in Freestyle-293 medium under serum-free conditions. In another example production at the 10-liter scale was performed in stirred-tank bioreactors at 27° C. and 80 rpm with dissolved oxygen level maintained at 50% of oxygen in air and pH maintained between 7.0 and 7.2. For both subunits the cells were transduced using a total MOI of 10 (75% alpha and 25% beta) and the culture supernatant was harvested at 96 hours post-transduction using centrifugation and filtration through a 0.2 micron filter.

The supernatant from a 10-liter culture was placed in a stir-jar and 25 mL of Nickel-SEPHAROSE Excel resin (GE) was added. The supernatant was stirred overnight at 25 rpm and 4° C. and the resin was collected into a column (26 mm×60 mm) and washed with 8 CV of 2×DPBS pH 7.4. The glucosidase II protein was eluted using a linear gradient of 0-60% 2×DPBS+500 mM imidazole pH 7.4. Fractions (10 mL) were collected and analyzed by SDS PAGE and Western Blot (reducing anti-His and anti-Strep). Fractions containing purified glucosidase II were pooled and concentrated for application to a SUPERDEX 200 (26 mm×600 mm) column (GE). The column buffer was PBS pH 7.2 and the concentrated eluate pool was loaded at a rate of 1 mL per minute. Fractions (3 mL) were collected and analyzed using SDS PAGE and Western Blot (reducing anti-His and anti-Strep). Fractions containing purified glucosidase II in PBS pH 7.2 were pooled and concentrated to 1 mg/mL. The final product was filter sterilized and stored at 4° C.

Alpha-glucosidase II Assay: The assay was performed by incubating recombinant enzyme alpha-glucosidase II for 60 minutes at 37° C. with multiple dilutions of test compounds. 4-methylumbelliferyl-alpha-D-pyranoside was then introduced as the substrate to the mixture. Fluorogenic 1,4-methyllumbelliferone was generated, and the reaction was stopped with the stop solution (0.5M glycine, 0.3M NaOH pH 10) after an incubation of 30 minutes at 37° C. 1,4-methyllumbelliferone was detected by fluorescence (excitation at 365 nm, emission at 440 nm). Percent inhibition was then plotted as a function of concentration for each test compound. The 50% inhibitory concentration (IC₅₀) is determined using a 4-PL curve fit and serves as a measure of relative inhibitory activity of each test compound (see Table 3).

Acid alpha-glucosidase (GAA): To analyze inhibitory effects of a test compound, commercially available recombinant human GAA was incubated with multiple dilutions of test compound for 60 minutes at 37° C. 4-methylumbelliferyl-alpha-D-pyranoside was then introduced to the mixture as the substrate. 1,4-methyllumbelliferone is generated, and after an incubation for 20 minutes at 37° C., the reaction was stopped with the stop solution (0.5M glycine 0.3M NaOH pH 10). 1,4-methyllumbelliferone is detected by fluorescence (excitation at 365 nm, emission at 440 nm). Relative fluorescence compared to untreated enzymatic activity and test compound concentration is plotted as a function of percent inhibition. The IC₅₀ is determined using a 4-PL curve fit as the measure of relative inhibitory activity of each test compound (see Table 3).

Beta-glucocerebrosidase (GBA, also referred to glucosylceramidase): To analyze inhibitory effects of a test compound, commercially available recombinant human GBA was incubated with multiple dilutions of test compound for 60 minutes at 37° C. 4-methylumbelliferyl-beta-D-pyranoside was then introduced to the mixture as substrate. Following incubation for 60 minutes at 37° C., 1,4-methyllumbelliferone was generated, and the reaction was stopped with the stop solution (0.5 M glycine; 0.3 M NaOH, pH 10). 1,4-methyllumbelliferone was detected by fluorescence (excitation at 365 nm, emission at 440 nm). Relative fluorescence compared to untreated enzymatic activity and test compound concentration is then plotted as a function of percent inhibition. The IC₅₀ was determined using a 4-PL curve fit as the measure of relative inhibitory activity of each test compound (see Table 3).

Intestinal alpha-glucosidases (rat maltase and sucrase): Intestinal alpha-glucosidases act upon 1,4-alpha-glucoside bonds, breaking down disaccharides to glucose. To perform this assay, partially purified alpha-glucosidase from intestinal rat powder was incubated for 60 minutes at 37° C. with multiple dilutions of test compounds. Maltose or Sucrose respectively was then introduced as substrate to the mixture and incubated for 30 minutes at 37° C. The reaction was stopped by a 5-minute incubation at 90° C. and the resultant glucose production is detected using Sigma's Glucose GO Kit. Percent inhibition was then plotted as a function of concentration for each test compound. The IC₅₀ was determined using a 4-PL curve fit and serves as the measure of relative inhibition of each test compound (see Table 3).

Table 3, below, includes the inhibition (neutralizing of activity) of compounds comprising general Formula (I) or Formula (II) for sucrase, maltase, acid alpha-glucosidase (GAA), beta-glucocerebrosidase (GBA), Glucosidase 1 (Glu 1), and Glucosidase 2 (Glu 2).

TABLE 3
Inhibition (% neutralizing of activity)

| | Sucrase | Maltase | GAA | GBA | Glu 1 | Glu2 |
|---|---|---|---|---|---|---|
| # | IC₅₀ (μM) | | | | | |
| 1001 | 0.015 | <0.001 | 0.091 | 99.74 | 0.013 | 0.437 |
| 1003 | 0.382 | 0.021 | 0.299 | 7.676 | 0.366 | 0.002 |
| 1005 | 0.661 | 0.058 | 0.802 | >100 | 0.404 | 1.583 |
| 1006 | 0.116 | 0.010 | 0.124 | 0.618 | 0.220 | 0.001 |
| 1008 | 0.078 | 0.006 | 0.064 | 2.327 | 0.103 | 0.012 |
| 1009 | 0.071 | 0.007 | 0.062 | 2.586 | 0.163 | 0.017 |
| 1010 | 0.096 | 0.012 | 0.359 | >100 | 0.199 | 1.068 |
| 1012 | 0.033 | 0.009 | 0.350 | 32.26 | 0.018 | 1.320 |
| 1013 | 0.026 | 0.005 | 0.254 | >100 | 0.053 | 0.153 |
| 1014 | 0.015 | 0.006 | 0.030 | >100 | 0.001 | 0.004 |
| 1015 | 0.078 | 0.008 | 0.129 | >100 | 0.009 | 0.002 |
| 1018 | <0.001 | <0.001 | 0.044 | >100 | <0.001 | <0.001 |
| 1020 | 0.129 | 0.044 | 1.157 | 76.30 | 0.118 | ND |
| 1021 | 0.001 | 0.008 | 2.090 | 25.82 | 0.006 | 34.98 |
| 1022 | 0.026 | 0.028 | 0.803 | 90.35 | 0.019 | 7.917 |
| 1023 | 0.066 | 0.030 | 1.438 | 91.09 | 0.015 | 42.56 |
| 1024 | 0.014 | 0.002 | 0.221 | 21.10 | 0.008 | ND |
| 1025 | 0.002 | 0.004 | 0.135 | 38.95 | 0.021 | 0.229 |
| 1026 | <0.001 | <0.001 | 0.064 | 37.92 | 0.007 | <0.001 |
| 1027 | 0.023 | 0.081 | 2.831 | 75.74 | 0.022 | 23.60 |
| 1028 | 0.011 | 0.011 | 0.666 | 12.91 | <0.001 | 0.827 |
| 1029 | <0.001 | 0.010 | 1.333 | 52.30 | 0.008 | 20.43 |
| 1030 | <0.001 | <0.001 | 0.135 | 32.30 | <0.001 | <0.001 |
| 1031 | 0.288 | 0.174 | 5.219 | >100 | 0.093 | 10.74 |
| 1032 | 0.036 | 0.073 | 4.175 | >100 | 0.016 | 47.72 |
| 1033 | 0.034 | 0.037 | 5.366 | >100 | 0.009 | >100 |
| 1034 | 0.057 | 0.033 | 2.709 | 91.9 | 0.020 | 10.04 |
| 1035 | 0.051 | 0.049 | 5.096 | >100 | 0.044 | 66.54 |
| 1036 | 0.219 | 0.077 | 2.761 | >100 | 0.030 | 49.52 |
| 1040 | 0.126 | 0.038 | 1.302 | >100 | 0.017 | 4.605 |
| 1041 | 0.187 | 0.079 | 1.884 | >100 | 0.017 | 7.178 |
| 1042 | 0.163 | 0.029 | 1.826 | >100 | 0.03 | 8.676 |
| 1044 | <0.001 | <0.001 | 1.419 | 53.18 | 0.003 | 2.673 |
| 1045 | <0.001 | <0.001 | 1.963 | 60.74 | <0.001 | 7.702 |
| 1046 | 0.052 | 0.006 | 1.635 | 87.31 | 0.017 | 17.96 |
| 1047 | 0.011 | <0.001 | 1.469 | 87.34 | 0.013 | 12.90 |
| 1048 | 0.059 | 0.013 | 0.366 | >100 | 0.145 | 0.55 |
| 1050 | 0.004 | <0.001 | 0.278 | >100 | 0.032 | 0.003 |
| 1051 | <0.001 | <0.001 | 0.291 | 15.00 | 0.048 | 0.213 |
| 1052 | 0.060 | 0.014 | 0.150 | 84.54 | 0.073 | 0.179 |
| 1053 | 0.023 | 0.002 | 0.118 | >100 | 0.104 | 0.139 |
| 1054 | 0.160 | 0.057 | 0.371 | >100 | 0.102 | 2.202 |
| 1058 | 0.046 | 0.008 | 0.078 | >100 | 0.021 | 0.114 |
| 1059 | 0.045 | 0.008 | 0.074 | >100 | 0.033 | 0.058 |
| 1060 | 0.052 | 0.009 | 0.086 | >100 | 0.041 | 0.262 |
| 1062 | <0.001 | <0.001 | 0.068 | 35.45 | 0.001 | <0.001 |
| 1063 | <0.001 | <0.001 | 0.032 | >100 | 0.001 | 0.07 |
| 1064 | 0.026 | 0.002 | 0.039 | 53.66 | 0.012 | 0.002 |
| 1065 | 0.037 | 0.003 | 0.090 | 32.13 | 0.009 | 0.006 |
| 1066 | 0.026 | 0.041 | 1.059 | >100 | 0.069 | 3.356 |
| 1067 | 0.080 | 0.081 | 0.426 | 3.725 | 0.099 | 12.81 |
| 1068 | 0.141 | 0.049 | 0.386 | >100 | 0.122 | 1.578 |
| 1069 | 0.008 | <0.001 | 0.068 | 3.092 | 0.022 | 0.004 |
| 1075 | 0.722 | 0.238 | 3.728 | 2.519 | 1.919 | 0.150 |
| 1076 | 0.854 | 0.250 | 4.263 | 7.813 | 1.307 | 0.066 |
| 1077 | 8.136 | 6.650 | 10.04 | 0.379 | 0.697 | 0.203 |
| 1078 | 0.506 | 0.028 | 0.343 | 6.974 | 0.200 | 0.095 |
| 1081 | 0.161 | 0.11 | 1.274 | >100 | 0.169 | 70.44 |
| 1082 | 0.025 | 0.014 | 0.872 | 13.80 | 0.069 | 5.630 |
| 1083 | 0.028 | 0.054 | 1.955 | >100 | 0.072 | 9.993 |
| 1084 | 0.044 | 0.095 | 2.347 | >100 | 0.079 | 26.56 |
| 1086 | 0.076 | 0.124 | 0.976 | >100 | 0.132 | 44.21 |
| 1087 | <0.001 | 0.004 | 0.554 | >100 | <0.001 | 8.110 |
| 1088 | <0.001 | <0.001 | 1.946 | 68.41 | <0.001 | 6.358 |
| 1089 | 0.023 | 0.036 | 0.572 | 97.14 | 0.026 | 20.72 |
| 1090 | 0.002 | 0.034 | 0.509 | 32.35 | 0.007 | 13.82 |
| 1092 | 0.048 | 0.038 | 0.786 | 120.8 | 0.059 | 1.781 |
| 1093 | 0.007 | 0.002 | 0.108 | 7.480 | 0.012 | 1.038 |
| 1094 | 0.057 | 0.019 | 0.246 | 12.29 | 0.028 | 0.177 |
| 1095 | 0.013 | 0.011 | 0.243 | 100 | 0.012 | 0.657 |
| 1096 | 0.023 | 0.011 | 0.259 | 17.62 | 0.015 | 1.429 |
| 1098 | 0.040 | 0.013 | 0.175 | 100 | 0.023 | 0.083 |
| 1099 | <0.001 | <0.001 | <0.001 | 37.13 | <0.001 | <0.001 |
| 1100 | 0.002 | <0.001 | 0.161 | 41.68 | <0.001 | <0.001 |
| 1101 | 0.004 | <0.001 | 0.025 | 32.68 | <0.001 | 0.043 |
| 1102 | <0.001 | <0.001 | 0.028 | 28.67 | <0.001 | 0.064 |
| 1103 | 0.569 | 0.279 | 2.664 | 11.90 | 0.267 | 0.005 |
| 1105 | 0.012 | 0.006 | 0.060 | 9.088 | 0.016 | 0.151 |
| 1106 | 0.006 | 0.031 | 0.036 | 6.969 | <0.001 | 0.009 |
| 1107 | 0.576 | 0.249 | 3.191 | 0.362 | 0.107 | 0.041 |
| 1108 | 0.908 | 0.098 | 0.311 | 5.997 | 0.247 | 0.007 |
| 1109 | 0.446 | 0.001 | 0.003 | 6.545 | 0.006 | <0.001 |
| 1110 | 0.063 | <0.001 | 0.007 | 0.048 | <0.001 | <0.001 |
| 1111 | 0.773 | <0.001 | 0.020 | 1.791 | 0.068 | 0.01 |
| 1112 | 0.915 | 0.001 | 0.105 | 4.985 | 0.089 | 0.101 |
| 1113 | 0.074 | 0.020 | 0.380 | 6.290 | 1.010 | 0.296 |
| 1114 | 0.052 | 0.005 | 0.016 | 5.141 | 0.108 | 0.003 |
| 1115 | 10.53 | 6.742 | 5.491 | 0.407 | 0.100 | 1.075 |
| 1116 | 1.229 | 0.162 | 0.428 | 0.904 | 1.361 | <0.001 |
| 1117 | 0.483 | 0.218 | 0.498 | 18.08 | 0.877 | 0.139 |
| 1118 | 0.058 | 0.004 | 0.027 | 2.434 | 0.010 | 0.005 |
| 1120 | 0.506 | 0.186 | 0.581 | 0.013 | 1.133 | 0.001 |
| 1121 | 0.868 | 0.224 | 1.011 | 0.069 | 0.879 | <0.001 |
| 1122 | 1.662 | 0.717 | 0.558 | 0.006 | 1.159 | 0.294 |
| 1123 | 2.810 | 0.440 | 0.700 | 0.627 | 1.360 | 0.329 |
| 1124 | 0.147 | 0.023 | 0.036 | 3.000 | 0.513 | 0.018 |
| 1125 | 0.092 | 0.015 | 0.080 | 9.688 | 0.337 | 0.019 |
| 1126 | 13.47 | 0.023 | 0.086 | 7.041 | 0.550 | 0.035 |
| 1128 | 0.039 | <0.001 | 0.005 | 5.195 | 0.116 | 0.001 |
| 1129 | 0.056 | 0.005 | 0.028 | 2.732 | 0.148 | 0.007 |
| 1130 | 0.030 | 0.004 | 0.012 | 0.974 | 0.244 | 0.006 |
| 1131 | 0.040 | 0.008 | 0.025 | 0.308 | 0.232 | 0.003 |
| 1133 | 0.237 | 0.097 | 0.542 | 4.501 | 1.280 | 0.005 |
| 1134 | 0.259 | 0.130 | 0.167 | 2.361 | 0.640 | 0.001 |
| 1136 | <0.001 | <0.001 | 0.009 | 0.037 | 0.025 | 0.001 |
| 1138 | <0.001 | <0.001 | <0.001 | 0.018 | 0.115 | 0.002 |
| 1140 | 4.144 | 3.119 | 2.089 | 0.674 | 0.153 | 0.045 |
| 1141 | 0.696 | 0.043 | 0.188 | 8.062 | 1.159 | 0.139 |
| 1143 | 2.013 | 2.056 | 0.796 | 0.404 | 0.334 | 0.002 |
| 1145 | 0.003 | <0.001 | 0.060 | 0.458 | 0.283 | <0.001 |
| 1146 | 0.092 | 0.023 | 0.068 | 2.173 | 0.511 | 0.015 |
| 1147 | 0.04 | 0.008 | 0.038 | 1.977 | 0.263 | 0.034 |
| 1148 | 0.001 | <0.001 | 0.088 | 0.002 | 0.372 | <0.001 |
| 1149 | <0.001 | <0.001 | 0.066 | 0.056 | 0.264 | <0.001 |
| 1150 | <0.001 | <0.001 | 0.045 | 0.193 | 0.08 | <0.001 |
| 1151 | <0.001 | <0.001 | 0.024 | 0.299 | 0.099 | <0.001 |
| 1153 | 2.498 | 0.321 | 0.057 | 1.093 | 1.364 | 0.544 |
| 1154 | 2.879 | 0.624 | 0.296 | 1.702 | 0.919 | 0.002 |
| 1155 | 0.726 | 0.418 | 0.455 | 3.544 | 1.25 | <0.001 |
| 1156 | 0.610 | 0.287 | 0.814 | 7.134 | 1.267 | 0.118 |
| 1157 | 0.228 | 0.107 | 0.292 | 4.589 | 0.336 | 0.004 |
| 1159 | 0.847 | 0.025 | <0.001 | 1.046 | 0.183 | 0.091 |
| 1160 | 0.931 | 0.075 | 0.024 | 1.862 | 1.569 | ND |
| 1161 | 0.74 | 0.273 | 0.084 | 2.823 | 0.729 | 0.002 |
| 1162 | 0.476 | 0.138 | 0.062 | 2.682 | 0.508 | <0.001 |
| 1163 | 0.584 | 0.309 | 0.093 | 6.818 | 0.211 | <0.001 |
| 1166 | 0.156 | <0.001 | 1.382 | 5.634 | 0.184 | 2.009 |
| 1169 | <0.001 | <0.001 | 0.420 | 83.51 | 0.068 | 16.09 |
| 1170 | 0.002 | <0.001 | 0.081 | 29.92 | 0.004 | <0.001 |
| 1171 | 10.65 | 5.657 | 2.076 | 0.204 | 0.301 | 1.014 |
| 1173 | 0.456 | 0.048 | 1.798 | 5.324 | 0.157 | 0.666 |
| 1174 | 0.001 | <0.001 | 0.230 | 0.805 | 0.531 | <0.001 |
| 1177 | 0.883 | 0.032 | 5.536 | 3.318 | 0.374 | 0.065 |
| 1178 | 71.51 | 3.482 | 5.934 | 5.901 | 2.733 | 9.364 |
| 1179 | 0.181 | 0.001 | 0.692 | 0.380 | <0.001 | <0.001 |
| 1182 | 0.179 | 0.026 | 0.033 | 6.130 | 0.047 | 0.002 |
| 1183 | 0.094 | 0.015 | 0.090 | 5.615 | 0.154 | 0.004 |
| 1184 | 0.065 | 0.006 | 0.048 | 0.950 | 0.084 | 0.001 |
| 1186 | 0.175 | 0.005 | 0.263 | 1.284 | 0.003 | 0.001 |
| 1187 | 2.048 | 1.205 | 0.615 | 16.38 | 0.094 | 0.003 |
| 1188 | 0.783 | 0.164 | 0.838 | 9.046 | 0.762 | 1.142 |
| 1191 | 2.012 | 0.171 | 2.738 | 28.58 | 7.549 | 20.28 |
| 1196 | 0.175 | 0.002 | 0.010 | 0.773 | 0.105 | 0.002 |
| 1197 | 0.177 | 0.009 | 0.037 | 2.074 | 0.140 | 0.001 |
| 1198 | 0.001 | <0.001 | 0.047 | 2.704 | 0.012 | 0.017 |
| 1199 | 0.016 | 0.008 | 0.043 | 3.96 | 0.077 | 0.012 |
| 1200 | 0.746 | 0.081 | 1.157 | 1.446 | 0.031 | 0.006 |
| 1202 | <0.001 | <0.001 | 0.039 | 3.007 | <0.001 | <0.001 |
| 1203 | 0.011 | <0.001 | 0.040 | 0.787 | 0.003 | <0.001 |
| 1207 | 0.071 | 0.007 | 0.075 | 1.681 | 0.118 | 0.014 |
| 1211 | 0.072 | 0.001 | 0.016 | 0.326 | 0.112 | 0.002 |
| 1212 | 0.089 | 0.002 | 0.047 | 0.386 | 0.105 | 0.005 |
| 1216 | 0.796 | 0.008 | 0.037 | 2.137 | 0.067 | 0.002 |
| 1217 | 0.225 | 0.011 | 0.010 | 0.981 | 0.128 | 0.011 |
| 1219 | 0.064 | 0.01 | 0.089 | 8.674 | 0.093 | 0.018 |
| 1221 | 0.424 | 0.233 | 4.511 | 0.057 | 2.231 | 0.220 |
| 1226 | 0.793 | 4.627 | 20.94 | 3.210 | 9.534 | 1.348 |
| 1227 | 0.104 | 0.012 | 0.082 | 0.592 | 0.169 | 0.017 |
| 1228 | 0.061 | 0.001 | 0.068 | 0.838 | 0.019 | 0.011 |
| 1229 | 0.070 | 0.024 | 0.081 | 0.959 | 0.094 | 0.037 |
| 1231 | 0.067 | 0.030 | 0.091 | 2.112 | 0.187 | 0.030 |
| 1233 | 0.130 | 0.032 | 1.035 | 1.308 | 0.123 | 0.023 |
| 1235 | 8.649 | 7.156 | 37.82 | 11.38 | 8.108 | 0.530 |
| 1236 | 0.254 | 0.036 | 0.556 | 4.630 | 0.296 | 0.039 |
| 1239 | 0.294 | <0.001 | 1.236 | 0.114 | 1.437 | 0.108 |
| 1240 | 0.318 | 0.701 | 12.76 | 0.496 | 0.283 | 0.045 |
| 1245 | 0.120 | 0.032 | 0.392 | 56.00 | 0.292 | 0.051 |
| 1246 | >100 | >100 | 0.046 | 21.57 | 0.112 | 0.004 |
| 1247 | 0.216 | 0.018 | 0.053 | 11.84 | 0.493 | 0.006 |
| 1249 | 1.868 | 3.486 | 1.92 | 0.320 | 2.446 | 0.162 |
| 1251 | 0.038 | 0.004 | 0.041 | 1.412 | 0.226 | 0.012 |
| 1252 | 0.080 | 0.037 | 0.141 | 8.671 | 1.350 | 0.082 |
| 1253 | 3.439 | 0.777 | 0.664 | >100 | 1.750 | 0.225 |
| 1254 | 6.532 | 1.209 | >100 | >100 | 6.770 | 0.865 |
| 1255 | 0.056 | 0.033 | 0.228 | 3.085 | 2.010 | 0.029 |
| 1256 | 0.088 | 0.010 | 0.033 | 1.230 | 0.090 | 0.006 |
| 1257 | 0.070 | 0.008 | 0.073 | 6.532 | 0.115 | 0.019 |
| 1258 | 0.108 | 0.019 | 0.032 | 1.349 | 0.032 | 0.031 |
| 1259 | 0.417 | 0.020 | 0.238 | 1.352 | 0.330 | 0.072 |
| 1260 | 0.029 | <0.001 | 0.044 | 1.563 | 0.136 | 0.015 |
| 1265 | 0.098 | 0.005 | 0.035 | 5.082 | 0.282 | 0.012 |
| 1266 | 0.140 | 0.002 | 0.103 | 6.342 | 0.180 | 0.007 |
| 1268 | 0.363 | 0.068 | 0.617 | 1.522 | 1.990 | 0.032 |
| 1270 | 0.350 | 0.057 | 0.190 | 0.081 | 0.358 | 0.046 |
| 1271 | 0.266 | 0.033 | 1.365 | 1.901 | 1.070 | 0.539 |
| 1272 | 1.876 | 0.239 | 0.800 | 4.102 | 1.800 | 0.039 |
| 1273 | 21.61 | 0.646 | 15.64 | 2.979 | 13.00 | 0.548 |
| 1274 | 0.738 | 0.151 | 0.136 | 2.988 | 1.550 | 0.048 |
| 1275 | 0.413 | 0.288 | 0.556 | 2.174 | 4.064 | 0.366 |
| 1276 | 0.316 | 0.049 | 0.253 | 0.062 | 2.440 | 0.039 |
| 1277 | 0.133 | 0.052 | <0.01 | <0.01 | 0.166 | <0.001 |
| 1280 | ND | ND | ND | ND | 1.457 | 0.194 |
| 1290 | <0.01 | <0.01 | 0.021 | 0.175 | 0.036 | 0.0012 |
| 1292 | 0.040 | 0.011 | 0.028 | 1.604 | 0.157 | 0.0086 |
| 1296 | 0.494 | <0.01 | 0.124 | 3.263 | 0.142 | 0.029 |
| 1297 | 0.800 | 0.169 | 0.452 | 3.520 | 1.302 | 0.097 |
| 1299 | 0.037 | 0.005 | 0.032 | 3.386 | 0.189 | 0.017 |
| 1300 | 0.052 | < 0.01 | 0.020 | 0.026 | 0.157 | 0.014 |
| 1304 | 0.060 | 0.017 | 0.043 | 0.043 | 0.178 | 0.022 |
| 1305 | 1.009 | <0.01 | 0.498 | 7.860 | 2.831 | <0.001 |
| 1306 | <0.01 | <0.01 | 4.569 | >100 | 0.745 | 44.02 |
| 1307 | 0.387 | 0.032 | 0.950 | 5.000 | 1.162 | 0.077 |
| 1308 | 0.331 | 0.088 | 1.685 | 50.87 | >100 | 0.125 |
| 1309 | 0.155 | 0.022 | 0.263 | 2.341 | 0.700 | 0.037 |
| 1310 | 0.371 | < 0.01 | 1.187 | 16.89 | 0.678 | 0.078 |
| 1311 | 1.009 | < 0.01 | 0.747 | 26.24 | 1.761 | 0.197 |
| 1315 | 0.044 | 0.009 | 0.440 | >100 | 0.040 | 0.907 |
| 1316 | 0.014 | 0.006 | 0.094 | >100 | 0.016 | 0.194 |
| 1317 | <0.001 | <0.001 | 0.480 | 45.10 | <0.001 | 1.2 |
| 1322 | 0.481 | 0.388 | 1.108 | 98.96 | 3.122 | 32.36 |
| 1323 | 0.037 | 0.011 | 0.076 | >100 | 0.011 | 0.382 |
| 1324 | <0.001 | <0.001 | 0.080 | 33.74 | <0.001 | 0.318 |
| 1329 | 0.675 | 0.548 | 0.050 | 82.91 | 1.871 | 2.550 |
| 1330 | 0.595 | 0.577 | 0.090 | 13.15 | 1.421 | 14.41 |
| 1332 | 3.495 | 0.573 | 0.165 | >100 | 2.821 | >100 |
| 1333 | 1.585 | 0.846 | 2.182 | >100 | 4.532 | >100 |
| 1334 | 0.336 | 0.224 | 0.714 | >100 | 1.078 | >100 |
| 1335 | 0.321 | 0.231 | 0.470 | 21.54 | 0.966 | >100 |
| 1336 | 21.32 | 9.991 | 10.40 | >100 | 30.32 | >100 |
| 1338 | 0.371 | 0.146 | 0.860 | 9.437 | 0.999 | 0.109 |
| 1339 | 0.049 | 0.014 | 0.240 | 0.626 | 0.078 | 0.194 |

*ND is not determined

1. A compound comprising Formula (I): 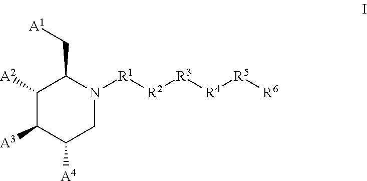
wherein
A¹, A², A³, and A⁴ are independently selected from the group consisting of hydrogen, hydroxyl, and hydroxyl-protecting group;
R¹ is absent or substituted or unsubstituted C₁-C₆ alkyl;
R² is selected from the group consisting of absent, NH, and 0;
R³ is selected from the group consisting of substituted or unsubstituted amine, sulfonyl, substituted or unsubstituted C₂-C₆ alkyl, substituted or unsubstituted C₁-C₆ heteroalkyl, substituted or unsubstituted C₁-C₆ alkylene, substituted or unsubstituted C₁-C₆ alkyne, substituted or unsubstituted C₅-C₁₂ aryl, substituted or unsubstituted C₃-C₁₂ cycloalkyl, substituted or unsubstituted C₃-C₁₂ heterocycloalkyl, substituted or unsubstituted C₃-C₁₂ heterocyclyl, substituted or unsubstituted C₅-C₁₂ heteroaryl, substituted or unsubstituted C₃-C₁₂ heterocyclyl, and substituted or unsubstituted C₃-C₁₂ aralkyl;
R⁴ is selected from the group consisting of absent, substituted or unsubstituted amine, substituted or unsubstituted C₁-C₆ alkyl, and substituted or unsubstituted C₁-C₆ heteroalkyl;
R⁵ is selected from the group consisting of absent, NH, NCH₃, and O;
R⁶ is 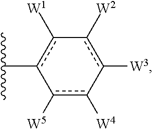
wherein optionally, at least one CW group in 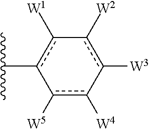
is replaced with N, O, or NH;
wherein [symbol]
are independently a single bond or are absent;
each of W¹, W², W³, W⁴, and W⁵ are independently selected from the group consisting of hydrogen, halogen, hydroxyl, alkoxy, nitro, nitrile, imide, imine, amide, sulfonamide, amino, cyanate, carboxylic acid, substituted or unsubstituted amine, azide, sulfonyl, methoxy, C₁-C₆ alkoxy, substituted or unsubstituted sulfone, substituted or unsubstituted C₁-C₆ alkyl, substituted or unsubstituted C₁-C₆ alkenes, substituted or unsubstituted C₁-C₆ alkynes, substituted or unsubstituted C₁-C₆ heteroalkyl, substituted or unsubstituted C₅-C₁₂ aryl, substituted or unsubstituted C₅-C₁₂ heteroaryl, substituted or unsubstituted C₃-C₁₂ cycloalkyl, substituted or unsubstituted C₃-C₁₂ heterocycloalkyl, substituted or unsubstituted C₃-C₁₂ heterocyclyl, substituted or unsubstituted C₃-C₁₂ heterocyclyl, and substituted or unsubstituted C₃-C₁₂ aralkyl;
with the proviso that when R¹, R², R³, and R⁴ taken together are a substituted or unsubstituted C₂-C₆ alkyl linker and R⁵ is NH then W¹, W², W³, W⁴, and W⁵ are not independently selected from the group consisting of hydrogen, nitrile, nitro, amine, substituted or unsubstituted alkanoyl group, adamantyl, substituted or unsubstituted C₃-C₁₂ heterocycloalkyl, and substituted or unsubstituted C₃-C₁₂ heteroalkyl; and
with the proviso that when R¹, R², R³, and R⁴ taken together are a substituted or unsubstituted C₂-C₆ alkyl linker and R⁵ is O then W¹, W², W³, W⁴, and W⁵ are not independently selected from the group consisting of hydrogen and substituted or unsubstituted C₃-C₆ heteroaryl;
or a pharmaceutically acceptable salt.

2. The compound of claim 1, wherein A¹, A², A³, and A⁴ are independently selected from the group consisting of hydroxyl and hydroxyl-protecting group.

3. The compound of claim 1, wherein R¹ is absent or an unsubstituted C₁-C₄ alkyl.

4. The compound of claim 3, wherein R¹ is selected from the group consisting of absent, methyl, ethyl, propyl, and butyl.

5. The compound of claim 1, wherein R² is absent or NH.

6. The compound of claim 1, wherein R³ is selected from the group consisting of substituted or unsubstituted C₆-C₁₂ aryl, substituted or unsubstituted C₂-C₆ alkyl, substituted or unsubstituted C₁-C₆ alkyne, substituted or unsubstituted C₁-C₆ heteroalkyl, substituted or unsubstituted C₃-C₁₂ cycloalkyl, and substituted or unsubstituted C₃-C₁₂ heterocycloalkyl.

7. The compound of claim 6, wherein R³ is selected from the group consisting of substituted or unsubstituted amine, sulfonyl, substituted or unsubstituted C₆-C₁₂ aryl, unsubstituted C₂-C₄ alkyl, unsubstituted C₁-C₄ alkyne, unsubstituted C₁-C₆ heteroalkyl, substituted C₁-C₆ heteroalkyl, substituted or unsubstituted C₅-C₁₂ heteroaryl, substituted or unsubstituted C₅-C₁₂ heterocyclyl, and substituted or unsubstituted C₅-C₁₀ heteroaryl.

8. The compound of claim 7, wherein R³ is selected from the group consisting of oxygen, ethyl, propyl, butyl, phenyl, biphenyl, naphthalene, indole, chlorophenyl, methoxyphenyl, tert-butylphenyl, cyclohexylphenyl, diphenylmethane, isopropylbenzene, azetidine, thiophene, pyridine, pyrazine, pyrimidine, piperazine, imidazole, pyrazole, furan, thiazole, cyclohexylphenyl, 2,3-dihydro-1H-indene, 2,3-dihydrobenzo[b]1,4-dioxine, bicyclo[1.1.1]pentan-1-yl)methyl, benzothiazole, 1,4-dioxane, 1,2,3-triazole, 1,2,3,4-tetrahydronaphthalene, N(CH₂)₅OCH₃, N(CH₂)₂CONH₃, CHC(O)NH₂, C(O)C(O), NHC(O), SO₂, C(OH)C(OH), CH₂SO₂, NHSO₂NH, NHC(O)NH, OCH₂, and CH₂O.

9. The compound of claim 1, wherein R⁴ is selected from the group consisting of absent, unsubstituted amine, unsubstituted C₁-C₄ alkyl, and unsubstituted C₁-C₄ heteroalkyl.

10. The compound of claim 9, wherein R⁴ is selected from the group consisting of absent, methyl, ethyl, propyl, butyl, NH(CH₂)₂, and O(CH₂)₂.

11. The compound of claim 1, wherein R⁵ is selected from the group consisting of absent, NH, and NCH₃.

12. The compound of claim 1, wherein R⁶ is

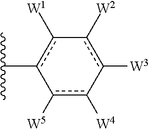

13. The compound of claim 12, wherein [symbol] are single bonds.

14. The compound of claim 12, wherein W¹, W², W³, W⁴, and W⁵ are independently selected from the group consisting of hydrogen, halogen, nitro, nitrile, carboxylic acid, cyanate, substituted amine, substituted or unsubstituted C₃-C₁₂ cycloalkyl, substituted or unsubstituted C₁-C₆ alkyl, substituted or unsubstituted C₁-C₆ heteroalkyl, substituted or unsubstituted sulfone, substituted or unsubstituted C₅-C₁₂ heteroaryl, substituted or unsubstituted C₅-C₁₂ heterocyclyl, and substituted or unsubstituted C₅-C₁₀ heteroaryl.

15. The compound of claim 14, wherein W¹, W², W³, W⁴, and W⁵ are independently selected from the group consisting of hydrogen, methyl, ethyl, propyl, nitro, aldehyde, bromo, chloro, fluoro, cyano, nitrile, methoxy, difluoromethyl, trifluoromethyl, tert-butyl, tert-butoxy, cyclopropyl, cyclohexyl, methanesulfonyl, methyltetrahydrofuran, hydromethyl, acetamide, N,N-dimethylacetamide, tert-butoxy, cyclohexene, cyclohexane, phenyl, tetrahydrofuran, pyridazine, morpholine, pyrrole, pyrimidine, furan, azide, methoxyethyl, oxazole, oxadiazole, imidazole, isoxazole, cyclopropanesulfonamide, sulfonylacetamide, (dimethylamino)methyl, methylcyano, anisole, phenyl, benzyl, N-chlorosuccinimide, 1,1-dimethoxyethane, 2,6-dimethylmorpholine, 2-oxa-5-azabicyclo[2.2.1]heptan-5-yl, 2-oxa-6-azaspiro[3.4]octan-6-yl, (1R,5S)-3-oxa-8-azabicyclo[3.2.1]octan-8-yl, 2-oxa-7-azaspiro[3.5]nonan-7-yl, methyl(2-nitro-4-[1,2-oxazolidin-2-yl])methyl, 2,3-dihydro-1,4-dioxine, (1,2-oxazolidin-2-yl)methyl, 2,3-dihydrofuran, 1,2,3-oxadiazole, 5-propyl-1,2,4-oxadiazole, 5-propyl,1,2,4-(1,2-oxazolidin-2-yl)methyl, 3,6-dihydro-2H-pyran, 5-phenyl-1,2,4-oxadiazole, 5-cyclobutyl-1,2,4-oxadiazole, 5-butyl-1,2,4-oxadiazole, 1,2,3-triazole, 2-oxa-7-azaspiro[3.5]nonane, 2-oxa-6-azaspiro[3.4]octane, COOH, COOCH₃, CONHAc, C(NH)NH₂, NHCOCH₃, SO₂NS(CH₃)₂, C(O)NH₂, SO₂NHCOCH₃, SO₂NH₂, CONHCa, C(O)OCH₃, C(NH)C(O)H, CCCH₃, CH₂C(O)O(CH₂)₂CH₃, (CH₃)₂OH, NHCN, SCN, CH₂CCH, and NHSO₂CH₃.

16. The compound of claim 1, wherein
A¹, A², A³, and A⁴ are hydroxyl;
R¹ is CH₂;
R² is absent or CH;
R³ is phenyl, biphenyl, or (CH₂)₄;
R⁴ is CH₂;
R⁵ is NH;
R⁶ is 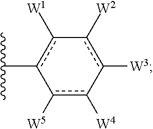 [symbol]
are single bonds; and
each of W¹, W², W³, W⁴, and W⁵ are independently selected from the group consisting of hydrogen, bromo, chloro, nitro, cyano, methyl, azide, methoxy, cyclohexene, pyrrole, furan, phenyl, pyridine, pyrimidine, pyridazine, oxazole, 1,4-dioxene, thiophene, CH₂-isoxazole, 3-oxa-8-azabicyclo[3.2.1]octanyl, 4-(5,6-dihydro-2H-thiopyran), 1,2,4-oxadiazole, 4-(pyrimidin-2-yl)morpholine, 3-(5-propyl-1,2,4-oxadiazole), 3-(5-cyclobutane-1,2,4-oxadiazole), 4-(piperidin-1-yl)pyrimidine, 4-(piperidin-1-yl)morpholine, 3,6-dihydro-2H-pyran, 2,3-dihydro-1,4-dioxine, 3-cyclopropylpyridazine, 5-propyl-1,2,4-oxadiazole, 5-cyclobutyl-1,2,4-oxadiazole, 1,2,4-oxadiazole, 3,6-dihydro-2H-thiopyran, 4-(pyrimidin-2-yl)morpholine, and 5-benzyl-1,2,4-oxadizaole.

17. A compound comprising Formula (II): 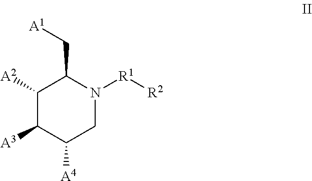
wherein
A¹, A², A³, and A⁴ are independently selected from the group consisting of hydrogen, hydroxyl, and hydroxyl-protecting group;
R¹ is absent or substituted or unsubstituted C₂-C₆ alkyl;
R² is 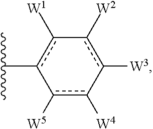
wherein optionally, at least one C or CW group in 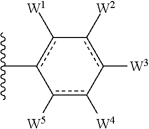
is replaced with N, O, or NH;
or 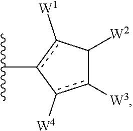
wherein optionally, at least one C or CW group in 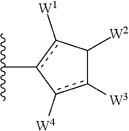
is replaced with N, O, or NH;
wherein [symbol]
are independently a single bond or are absent;
each of W¹, W², W³, W⁴, and W⁵ are independently selected from the group consisting of hydrogen, ketone, substituted or unsubstituted C₁-C₆ alkyl, and W¹ and W², W² and W³, W³ and W⁴, or W⁴ and W⁵ taken together form a substituted or unsubstituted C₅-C₆ aromatic ring;
or a pharmaceutically acceptable salt thereof.

18. The compound of claim 17, wherein A¹, A², A³, and A⁴ are hydroxyl.

19. The compound of claim 17, wherein R¹ is substituted or unsubstituted C₂-C₆ alkyl.

20. The compound of claim 19, wherein R¹ is a hexyl.

21. The compound of claim 17, wherein R² is

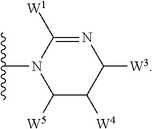

22. The compound of claim 17, wherein R² is

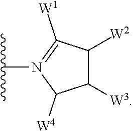

23. The compound of claim 17, wherein each of W¹, W², W³, W⁴, and W⁵ are independently selected from the group consisting of hydrogen, ketone, unsubstituted C₁-C₆ alkyl, and W¹ and W², W² and W³, or W³ and W⁴, W⁴ and W⁵ taken together form a substituted or unsubstituted C₅-C₆ aromatic ring.

24. The compound of claim 23, wherein each of W¹, W², W³, W⁴, and W⁵ are independently selected from the group consisting of hydrogen, ketone, methyl, and W¹ and W², W² and W³, W³ and W⁴, or W⁴ and W⁵ taken together form a substituted aryl.

25. The compound of claim 17, wherein the compound is selected from the group consisting of and 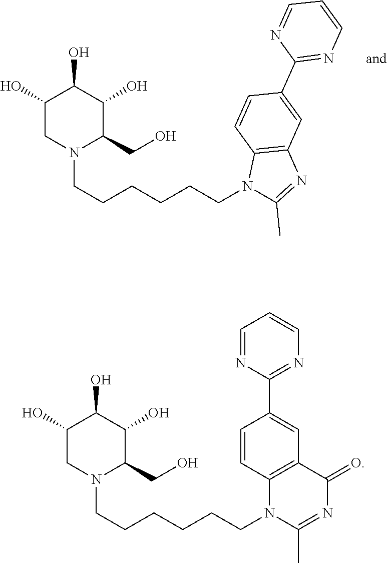 [SMILES: Cc1nc2cc(-c3ncccn3)ccc2n1CCCCCCN1C[C@H](O)[C@@H](O)[C@H](O)[C@H]1CO ; Cc1nc(=O)c2cc(-c3ncccn3)ccc2n1CCCCCCN1C[C@H](O)[C@@H](O)[C@H](O)[C@H]1CO]
